(19) United States
(12) Patent Application Publication
(10) Pub. No.: US 2024/0401018 A1
(43) Pub. Date: Dec. 5, 2024

(54) EVOLVED DOUBLE-STRANDED DNA DEAMINASE BASE EDITORS AND METHODS OF USE

(71) Applicants: The Broad Institute, Inc., Cambridge, MA (US); President and Fellows of Harvard College, Cambridge, MA (US)

(72) Inventors: David R. Liu, Cambridge, MA (US); Beverly Mok, Cambridge, MA (US)

(73) Assignees: The Broad Institute, Inc., Cambridge, MA (US); President and Fellows of Harvard College, Cambridge, MA (US)

(21) Appl. No.: 18/286,547

(22) PCT Filed: Apr. 12, 2022
(86) PCT No.: PCT/US2022/024499

§ 371 Date: Oct. 11, 2023

Related U.S. Application Data
(60) Provisional application No. 63/322,210, filed on Mar. 21, 2022.
(60) Provisional application No. 63/309,485, filed on Feb. 11, 2022.
(60) Provisional application No. 63/174,029, filed on Apr. 12, 2021.

(51) Int. Cl. C12N 9/78; C12N 9/22

(52) U.S. Cl. CPC C12N 9/78; C12N 9/22; C12Y 305/04005

(57) ABSTRACT

The specification provides programmable base editors that are capable of introducing a nucleotide change and/or which could alter or modify the nucleotide sequence at a target site in a double-stranded nucleotide sequence, such as, a chromosome, genome, or a mitochondrial DNA (mtDNA), with high specificity and efficiency. Moreover, the disclosure provides fusion proteins and compositions comprising a programmable DNA binding protein (e.g., a mitoTALE, a mitoZFP, or a CRISPR/Cas9) and evolved double-stranded DNA deaminase domains that is capable of being delivered to a cell nucleus and/or a mitochondria and carrying out precise installation of nucleotide changes in the target a double-stranded nucleotide sequence, such as, a chromosome, genome, or mtDNA. The fusion proteins and compositions are not limited for use with mtDNA, but may be used for base editing of any double-stranded target DNA.

### GOVERNMENT SUPPORT

This invention was made with government support under grant numbers AI080609, AI142756, HG009490, EB027793, EB031172, EB022376, GM122455, GM118062, DK089507, and GM095450 awarded by the National Institutes of Health, and grant number HDTRA1-13-1-0014 awarded by the Department of Defense. The government has certain rights in the invention.

### BACKGROUND OF THE INVENTION

Inherited or acquired mutations in mitochondrial DNA (mtDNA) can profoundly impact cell physiology and are associated with a spectrum of human diseases, ranging from rare inborn errors of metabolism,⁴ certain cancers,⁵ age-associated neurodegeneration,⁶ and even the aging process itself.^{7,8} Tools for introducing specific modifications to mtDNA are needed both for modeling diseases and for their therapeutic potential. The development of such tools, however, has been constrained in part by the challenge of transporting RNAs into mitochondria, including guide RNAs required to facilitate nucleic acid modification and/or editing using CRISPR-associated proteins.⁹

Each mammalian cell contains hundreds to thousands of copies of circular mtDNA.¹⁰ Homoplasmy refers to a state in which all mtDNA molecules are identical, while heteroplasmy refers to a state in which a cell contains a mixture of wild-type and mutant mtDNA. Current approaches to engineering and/or altering mtDNA rely on RNA-free DNA-binding proteins, such as transcription activator-like effectors nucleases (mitoTALENs)^{11,17} and zinc finger nucleases fused to mitochondrial targeting sequences (mitoZFNs), to induce double-strand breaks (DSBs).¹⁸⁻²⁰ Upon cleavage, the linearized mtDNA is rapidly degraded,²¹⁻²³ resulting in heteroplasmic shifts to favor uncut mtDNA genomes. As a candidate therapy however, this approach cannot be applied to homoplasmic mtDNA mutations²⁴ since destroying all mtDNA copies is presumed to be harmful.^{22,25} In addition, using DSBs to eliminate heteroplasmic mtDNA mutations, which tend to be functionally recessive,²⁶ implicitly requires the edited cell to restore its wild-type mtDNA copy number. During this transient period of mtDNA repopulation, the loss of mtDNA copies could cause cellular toxicity resulting in deleterious effects (e.g., apoptosis).

A favorable alternative to targeted destruction of DNA through DSBs is precision genome editing, a capability that has not yet been reported for mtDNA. The ability to precisely install or correct pathogenic mutations, rather than destroy targeted mtDNA, could accelerate our ability to model mtDNA diseases in cells and animal models, and in principle could also enable therapeutic approaches that correct pathogenic mtDNA and genomic DNA mutations.

Therefore, the development of programmable base editors that are capable of introducing a nucleotide change and/or which could alter or modify the nucleotide sequence at a target site with high specificity and efficiency within DNA, including genomic DNA and mtDNA, would substantially expand the scope and therapeutic potential of genome editing technologies.

### SUMMARY

The present disclosure is further to the inventors' discovery of a double-stranded DNA cytidine deaminase, referred to herein as “DddA,” and to its application in base editing of double-stranded nucleic acid molecules, and in particular, the editing of genomic and mitochondrial DNA, as described in Mok et al., “A bacterial cytidine deaminase toxin enables CRISPR-free mitochondrial base editing,” Nature, 2020; 583(7817): 631-637 (“Mok et al., 2020”), the entire contents of which are incorporated herein by reference. As depicted in FIG. 1A, the full-length naturally occurring DddA protein is toxic to cells. Without being bound by theory, this cellular toxicity may relate to the fact that the substrate of DddA is any double stranded DNA, including the chromosomal DNA. Thus, as described in Mok et al., the inventors found that the protein could be engineered into split DddA halves that are non-toxic to the cell and inactive on their own until brought together on a target DNA by adjacently bound programmable DNA-binding proteins (e.g., mitoTALE proteins, zinc finger proteins, or Cas9/sgRNA complexes) which bind to the DNA on either side of a site of deamination. The inventors proposed split sites within amino acid loop regions as identified by the crystal structure of DddA. They found that fusions of the split-DddA halves had the ability to deaminate double stranded DNA as a substrate when brought together at a site of deamination by a pair of programmable DNA binding proteins binding to different sites at a deamination site (or edit site).

As now disclosed herein, the inventors have used continuous evolution methods, including phage-assisted non-continuous evolution (PANCE) and phage-assisted continuous evolution (PACE), for example, as illustrated in FIG. 2, to evolve a starting point DddA protein or fragment thereof to form an evolved variant DddA or evolved fragment of DddA having one or more improved characteristics, including increased deaminase activity and/or expanded sequence contexts in which deamination may occur (e.g., expanding beyond the canonical DdCBE sequence context of TC, including non-TC contexts such as but not limited to AC and CC targets).

The present disclosure provides methods for making such DddA variants (e.g., evolution methods such as PANCE, PACE, or a combination thereof), methods of making base editors comprising said variants, base editors comprising fusion proteins of an evolved variant DddA and a programmable DNA binding protein (e.g., a mitoTALE, zinc finger, or napDNAbp), DNA vectors encoding said base editors, methods for delivery said based editors to cells, and methods for using said base editors to edit a target double stranded DNA molecule, including a mitochondrial genome or a genomic genome.

In the case of mitochondrial DNA (mtDNA), inherited or acquired mutations in mtDNA can profoundly impact cell physiology and are associated with a spectrum of human diseases, ranging from rare inborn errors of metabolism,⁴ certain cancers,⁵ age-associated neurodegeneration,⁶ and even the aging process itself.^{7,8} Tools for introducing specific modifications to mtDNA are urgently needed both for modeling diseases and for their therapeutic potential. The present disclosure provides such tools through the use of the newly discovered variants of the canonical DddA described herein in base editing of double-stranded DNA substrates, including genomic DNA, plasmid DNA, and mtDNA.

Each mammalian cell contains hundreds to thousands of copies of a circular mtDNA¹⁰. Homoplasmy refers to a state in which all mtDNA molecules are identical, while heteroplasmy refers to a state in which a cell contains a mixture of wild-type and mutant mtDNA. Current approaches to engineer mtDNA rely on DNA-binding proteins such as transcription activator-like effectors nucleases (mitoTALENs)^{11,17} and zinc finger nucleases (mitoZFNs)¹⁸⁻²⁰ fused to mitochondrial targeting sequences to induce double-strand breaks (DSBs). Such proteins do not rely on nucleic acid programmability (e.g., such as with Cas9 domains). Linearized mtDNA is rapidly degraded,²¹⁻²³ resulting in heteroplasmic shifts to favor uncut mtDNA genomes. As a candidate therapy however, this approach cannot be applied to homoplasmic mtDNA mutations²⁴ since destroying all mtDNA copies is presumed to be harmful.^{22,25} In addition, using DSBs to eliminate heteroplasmic mtDNA mutations, which tend to be functionally recessive,²⁶ implicitly requires the edited cell to restore its wild-type mtDNA copy number. During this transient period of mtDNA repopulation, the loss of mtDNA copies could result in cellular toxicity.

As described herein, the disclosure provides a platform of precision genome editing using an evolved double-stranded DNA deaminase (evolved DddA or DddA variant) and a programmable DNA binding protein, such as a TALE domain, zinc finger binding domain, or a napDNAbp (e.g., Cas9), to target the deamination of a target base, which through cellular DNA repair and/or replication, is converted to a new base, thereby installing a base edit at a target site. In some embodiments, the deaminase activity is a cytidine deaminase, which deaminates a cytidine, leading to a C-to-T edit at that site in a double-stranded DNA target (e.g., genomic DNA, plasmid DNA, or mtDNA). In some other embodiments, that deaminase activity is an adenosine deaminase, which deaminates an adenosine, leading to a A-to-G edit at that site. In various embodiments, the disclosure further relates to “split-constructs” and “split-delivery” of said constructs whereby to address the toxic nature of fully active DddA and DddA variants described herein when expressed inside cells (as discovered by the inventors), the DddA protein or DddA variant is “split” or otherwise divided into two or more DddA fragments which can be separately delivered, expressed, or otherwise provided to cells to avoid the toxicity of fully active DddA. Further, the DddA fragments may be delivered, expressed, or otherwise provided as separate fusion proteins to cells with programmable DNA binding proteins (e.g., zinc finger domains, TALE domains, or Cas9 domains) which are programmed to localize the DddA fragments to a target edit site, through the binding of the DNA binding proteins to DNA sites upstream and downstream of the target edit site. Once co-localized to the target edit site, the separately provided DddA fragments may associate (covalently or non-covalently) to reconstitute an active DddA protein or DddA variant with a double-stranded DNA deaminase activity. In certain embodiments where the objective is to base edit mitochondrial DNA targets, the programmable DNA binding proteins can be modified with one or more mitochondrial localization signals (MLS) so that the DddA-pDNAbp fusions or DddA variant-pDNAbp fusions are translocated into the mitochondria, thereby enabling them to act on mtDNA targets.

The inventors are believed to be the first to identify the herein disclosed DddA variants, as well as the canonical DddA, which was initially discovered as a bacterial toxin. The inventors further conceived of the idea of splitting the DddA variants into two or more domains, which apart do not have a deaminase activity (and as such, lack toxicity), but which may be reconstituted (e.g., inside the cell, and/or inside the mitochondria) to restore the deaminase activity of the protein. This allows the separate delivery DddA fragments to cells (and/or to mitochondria, specifically), or delivery of nucleic acid molecules expressing such DddA fragments to a cell, such that once present or expressed within a cell, DddA fragments may associate with one another. By “associate” it is meant the two or more DddA fragments may come into contact with one another (e.g., in a cell, at a target site in a genome, or within a mitochondria at a target mtDNA site) and form a functional DddA protein or variant within a cell (or mitochondria). The association of the two or more fragments may be through covalent interactions or non-covalent interactions. In addition, the DddA domains may be fused or otherwise non-covalently linked to a programmable DNA binding protein, such as a Cas9 domain or other napDNAbp domain, zinc finger domain or protein (ZF, ZFD, or ZFP), or a transcription activator-like effector protein (TALE), which allows for the co-localization of the two or more DddA fragments to a particular desired site in a target nucleic acid molecule which is to be edited, such that when the DddA fragments are co-localized at the desired editing site, they reform a functional DddA that is capable deaminating a target site on a double-stranded DNA molecule. In certain embodiments, the programmable DNA binding proteins can be engineered to comprise one or more mitochondrial localization signals (MLS) such that the DddA domains become translocated into the mitochondria, thereby providing a means by which to conduct base editing directly on the mitochondrial genome.

Accordingly, provided herein are compositions, kits, and methods of modifying double-stranded DNA (e.g., mitochondrial DNA or “mtDNA”) using genome editing strategies that comprise the use of a programmable DNA binding protein (“pDNAbp”) (e.g., a mitoTALE, mitoZFP, or a CRISPR/Cas9) and a double-stranded DNA deaminase (“DddA”) (e.g., a DddA variant of the canonical DddA) to precisely install nucleotide changes and/or correct pathogenic mutations in double-stranded DNA (e.g., genomic DNA, plasmid DNA, or mtDNA), rather than destroying the DNA (e.g., genomic DNA, plasmid DNA, or mtDNA) with double-strand breaks (DSBs). The present disclosure provides pDNAbp polypeptides, DddA polypeptides (e.g., DddA variants of canonical DddA), fusion proteins comprising pDNAbp polypeptides and DddA polypeptides (e.g, DddA variants of canonical DddA), nucleic acid molecules encoding the pDNAbp polypeptides, DddA polypeptides (e.g., DddA variants of canonical DddA), and fusion proteins described herein, expression vectors comprising the nucleic acid molecules described herein, cells comprising the nucleic acid molecules, expression vectors, pDNAbp polypeptides, DddA polypeptides (e.g., DddA variants of canonical DddA), and/or fusion proteins described herein, pharmaceutical compositions comprising the polypeptides, fusion proteins, nucleic acid molecules, vectors, or cells described herein, and kits comprising the polypeptides, fusion proteins, nucleic acid molecules, vectors, or cells described herein for modifying double-stranded DNA (e.g., mtDNA) by base editing.

The inventors now have used continuous evolution methods to construct novel DddA proteins (i.e., DddA variants of canonical DddA) which may be used in the base editors described herein to deaminate double-stranded DNA targets, such as genomic DNA, plasmid DNA, or mitochondrial DNA.

In some embodiments, the pDNAbps (e.g., a mitoTALE, mitoZFP, or a CRISPR/Cas) and the DddA variants described herein are expressed as fusion proteins. In other embodiments, the pDNAbps and DddA variants described herein are expressed as separate polypeptides. In various other embodiments, the fusion proteins and/or the separately expressed pDNAbps and DddAs become translocated into the mitochondria. To effect translocation, the fusion proteins and/or the separately expressed pDNAbps and DddA variants described herein can comprise one or more mitochondrial targeting sequences (MTS). To effect translocation in the nucleus in the case of genomic DNA editing, the fusion proteins and/or the separately expressed pDNAbps and DddA variants described herein can comprise one or more nuclear localization sequences (NLS).

In still other embodiments, the DddA variants described herein are administered to a cell in which base editing is desired as two or more polypeptide fragments, wherein each fragment by itself is inactive with respect to deaminase activity, but upon co-localization in the cell, e.g., inside the mitochondria or in the nucleus, the two or more fragments reconstitute the deaminase activity.

In certain embodiments, the reconstituted activity of the co-localized two or more fragments can comprise at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or at least 99.9% of the deaminase activity of a wildtype DddA or a DddA variant described herein.

In certain embodiments, the DddA (e.g., a DddA variant described herein) is separated into two fragments by dividing the DddA at a split site. A “split site” refers to a position between two adjacent amino acids (in a wildtype DddA amino acid sequence) that marks a point of division of a DddA. In certain embodiments, the DddA can have at least one split site, such that once divided at that split site, the DddA forms an N-terminal fragment and a C-terminal fragment. The N-terminal and C-terminal fragments can be the same or different sizes (or lengths), wherein the size and/or polypeptide length depends on the location or position of the split site. As used herein, reference to a “fragment” of DddA (or any other polypeptide) can be referred equivalently as a “portion.” Thus, a DddA which is divided at a split site can form an N-terminal portion and a C-terminal portion. Preferably, the N-terminal fragment (or portion) and the C-terminal fragment (or portion) or DddA do not have deaminase activity, or have a reduced deaminase activity that is reduced by at least 10%, or at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or up to 100% relative to the wild type DddA activity.

In various embodiments, a DddA may be split into two or more inactive fragments by directly cleaving the DddA at one or more split sites. Direct cleaving can be carried out by a protease (e.g., trypsin) or other enzyme or chemical reagent. In certain embodiments, such chemical cleavage reactions can be designed to be site-selective (e.g., Elashal and Raj, “Site-selective chemical cleavage of peptide bonds,” Chemical Communications, 2016, Vol. 52, pages 6304-6307, the contents of which are incorporated herein by reference.) In other embodiments, chemical cleavage reactions can be designed to be non-selective and/or occur in a random fashion.

In other embodiments, the two or more inactive DddA fragments can be engineered as separately expressed polypeptides. For instance, for a DddA having one split site, the N-terminal DddA fragment could be engineered from a first nucleotide sequence that encodes the N-terminal DddA fragment (which extends from the N-terminus of the DddA up to and including the residue on the amino-terminal side of the split site). In such an example, the C-terminal DddA fragment could be engineered from a second nucleotide sequence that encodes the C-terminal DddA fragment (which extends from the carboxy-terminus of the split site up to including the natural C-terminus of the DddA protein). The first and second nucleotide sequences could be on the same or different nucleotide molecules (e.g., the same or different expression vectors).

In various embodiments, the N-terminal portion of a split DddA variant may be referred to as “DddA-N half” and the C-terminal portion of a split DddA variant may be referred to as the “DddA-C half.” Reference to the term “half” does not connote the requirement that the DddA-N and DddA-C portions are identically half of the size and/or sequence length of a complete DddA, or that the split site is required to be at the midpoint of the complete DddA polypeptide. To the contrary, and as noted above, the split site can be between any pair of residues in the DddA polypeptide, thereby giving rise to half portions which are unequal in size and/or sequence length. In certain embodiments, the split site is within a loop region of a DddA variant described herein.

Accordingly, in one aspect, the disclosure relates to a pair of fusion proteins useful for making modifications to the sequence of mitochondrial DNA (e.g., mtDNA). The pair of fusion proteins, in some embodiments, can comprise a first fusion protein comprising a first pDNAbp (e.g., a mitoTALE, mitoZFP, or a CRISPR/Cas9) and a first portion or fragment of a DddA variant, and a second fusion protein comprising a second pDNAbp (e.g., mitoTALE, mitoZFP, or a CRISPR/Cas9) and a second portion or fragment of a DddA variant, such that the first and the second portions of the DddA variant reconstitute a DddA variant upon co-localization in a cell and/or mitochondria. In certain embodiments, that first portion of the DddA variant is an N-terminal fragment of the DddA variant and the second portion of the DddA variant is C-terminal fragment of the DddA variant. In other embodiments, the first portion of the DddA variant is a C-terminal fragment of the DddA variant and the second portion of the DddA variant is an N-terminal fragment of the DddA variant.

In this aspect, the structure of the pair of fusion proteins can be, for example:
[pDNAbp]-[DddA half^{A}] and [pDNAbp]-[DddA half^{B}];
[DddA-half^{A}]-[pDNAbp] and [DddA-half^{B}]-[pDNAbp];
[pDNAbp]-[DddA half^{A}] and [DddA-half^{B}]-[pDNAbp]; or
[DddA-half^{A}]-[pDNAbp] and [pDNAbp]-[DddA half^{B}], wherein “A” or “B” can be the N-terminal or C-terminal half of DddA.

In another aspect, the disclosure relates to a pair of fusion proteins useful for making modifications to the sequence of mitochondrial DNA (e.g., mtDNA). The pair of fusion proteins can comprise a first fusion protein comprising a first mitoTALE and a first portion or fragment of a DddA, and a second fusion protein comprising a second mitoTALE and a second portion or fragment of a DddA, such that the first and the second portions of the DddA, upon co-localization in a cell and/or mitochondria, are reconstituted as an active DddA. In certain embodiments, that first portion of the DddA is an N-terminal fragment of a DddA and the second portion of the DddA is C-terminal fragment of a DddA. In other embodiments, the first portion of the DddA is a C-terminal fragment of a DddA and the second portion of the DddA is an N-terminal fragment of a DddA. In this aspect, the structure of the pair of fusion proteins can be, for example:
[mitoTALE]-[DddA half^{A}] and [mitoTALE]-[DddA half^{B}];
[DddA-half^{A}]-[pDNAbp] and [DddA-half^{B}]-[mitoTALE];
[mitoTALE]-[DddA half^{A}] and [DddA-half^{B}]-[mitoTALE]; or
[DddA-half^{A}]-[mitoTALE] and [mitoTALE]-[DddA half^{B}], wherein “A” or “B” can be the N-terminal or C-terminal half of DddA.

In yet another aspect, the disclosure relates to a pair of fusion proteins useful for making modifications to the sequence of mitochondrial DNA (e.g., mtDNA). The pair of fusion proteins can comprise a first fusion protein comprising a first mitoZFP and a first portion or fragment of a DddA, and a second fusion protein comprising a second mitoZFP and a second portion or fragment of a DddA, such that the first and the second portions of the DddA, upon co-localization in a cell and/or mitochondria, are reconstituted as an active DddA. In certain embodiments, that first portion of the DddA is an N-terminal fragment of a DddA and the second portion of the DddA is C-terminal fragment of a DddA. In other embodiments, the first portion of the DddA is a C-terminal fragment of a DddA and the second portion of the DddA is an N-terminal fragment of a DddA. In this aspect, the structure of the pair of fusion proteins can be, for example:
[mitoZFP]-[DddA half^{A}] and [mitoZFP]-[DddA half^{B}];
[DddA-half^{A}]-[pDNAbp] and [DddA-half^{B}]-[mitoZFP];
[mitoZFP]-[DddA half^{A}] and [DddA-half^{B}]-[mitoZFP]; or
[DddA-half^{A}]-[mitoZFP] and [mitoZFP]-[DddA half^{B}], wherein “A” or “B” can be the N-terminal or C-terminal half of DddA.

In yet another aspect, the disclosure relates to a pair of fusion proteins useful for making modifications to the sequence of mitochondrial DNA (e.g., mtDNA). The pair of fusion proteins can comprise a first fusion protein comprising a first Cas9 domain and a first portion or fragment of a DddA, and a second fusion protein comprising a second Cas9 domain and a second portion or fragment of a DddA, such that the first and the second portions of the DddA, upon co-localization in a cell and/or mitochondria, are reconstituted as an active DddA. In certain embodiments, that first portion of the DddA is an N-terminal fragment of a DddA (i.e., “DddA half^{A}” as shown in FIGS. 1A-1E) and the second portion of the DddA is C-terminal fragment of a DddA (i.e., “DddA half^{B}” as shown in FIGS. 1A-1E). In other embodiments, the first portion of the DddA is a C-terminal fragment of a DddA and the second portion of the DddA is an N-terminal fragment of a DddA. In this aspect, the structure of the pair of fusion proteins can be, for example:
[Cas9]-[DddA half^{A}] and [Cas9]-[DddA half^{B}];
[DddA-half^{A}]-[Cas9] and [DddA-half^{B}]-[Cas9];
[Cas9]-[DddA half^{A}] and [DddA-half^{B}]-[Cas9]; or
[DddA-half^{A}]-[Cas9] and [Cas9]-[DddA half^{B}], wherein “A” or “B” can be the N-terminal or C-terminal half of DddA.

In each instance above of “]-[” can be in reference to a linker sequence.

In some embodiments, a first fusion protein comprises, a first mitochondrial transcription activator-like effector (mitoTALE) domain and a first portion of a DNA deaminase effector (DddA).

In some embodiments, the first portion of the DddA comprises an N-terminal truncated DddA. In some embodiments, the first mitoTALE domain is configured to bind a first nucleic acid sequence proximal to a target nucleotide. In some embodiments, the first portion of a DddA is linked to the remainder of the first fusion protein by the C-terminus of the first portion of a DddA.

In some embodiments, a second fusion protein comprises, a second mitoTALE domain and a second portion of a DddA. In some embodiments, the second portion of the DddA comprises a C-terminal truncated DddA. In some embodiments, the second mitoTALE domain is configured to bind a second nucleic acid sequence proximal to a nucleotide opposite the target nucleotide. In some embodiments, the second portion of a DddA is linked to the remainder of the second fusion protein by the C-terminus of the second portion of a DddA.

In some embodiments, the first or second fusion protein is the result of truncations of a DddA at a residue site selected from the group comprising: 62, 71, 73, 84, 94, 108, 110, 122, 135, 138, 148, and 155. In some embodiments, the first or second fusion protein is the result of truncations of a DddA at a residue 148.

In some embodiments, the first or second fusion protein further comprises a linker. In some embodiments, the linker is positioned between the first mitoTALE and the first portion of a DddA and/or between the second mitoTALE and the second portion of a DddA. In some embodiments, the linker is at least two amino acids and no greater than sixteen amino acid residues in length. In some embodiments, the linker is two amino acid residues.

In some embodiments, the first or second fusion protein further comprises at least one uracil glycosylase inhibitor. In some embodiments, the first or second fusion protein the at least one glycosylase inhibitor is attached to the C-terminus of the first and/or second portion of a DddA.

In another aspect, the disclosure relates to a pair of fusion proteins comprising: (a) a first fusion protein disclosed herein; and (b) a second fusion protein disclosed herein, wherein the first pDNAbp (e.g., mitoTALE, mitoZFP, or mitoCas9) of the first fusion protein is configured to bind a first nucleic acid sequence proximal to a target nucleotide and the second pDNAbp (e.g., mitoTALE, mitoZFP, or mitoCas9) of the second fusion protein is configured to bind a second nucleic acid sequence proximal to a nucleotide opposite the target nucleotide. In some embodiments, the first nucleic acid sequence of the pair of fusion proteins is upstream of the target nucleotide and the second nucleic acid of the pair of fusion proteins is upstream of a nucleic acid of the complementary nucleotide.

In another aspect the disclosure relates to a pair of fusion proteins, wherein the first and second fusion proteins disclosed herein, are configured to form a dimer, and dimerization of the first and second fusion proteins at closely spaced nucleic acid sequences reconstitutes at least partial activity of a full length DddA. In some embodiments, the dimerization of the pair of fusion proteins facilitates deamination of the target nucleotide.

In another aspect, the disclosure relates to a recombinant vector comprising an isolated nucleic acid as disclosed herein.

In some embodiments, the vector is part of a composition, the composition comprising the vector and a pharmaceutically acceptable excipient.

In another aspect, the disclosure relates to an isolated cell comprising a nucleic acid as disclosed. In some embodiments, the isolated cell is a mammalian cell. In some embodiments, the mammalian cell is a human cell.

In another aspect, the disclosure relates to a method of treating a subject having, at risk of having, or suspected of having, a disorder comprising administering an effective amount of a pair of fusion proteins as described herein, a nucleic acid as described herein, a vector as disclosed herein, a composition as described herein, and/or an isolated cell as described herein. For example, the disorder can be a mitochondrial disorder, such as, MELAS/Leigh syndrome or Leber's hereditary optic neuropathy.

In another aspect, the disclosure relates to a method of editing a nucleic acid in a subject, comprising: (a) determining a target nucleotide to be deaminated; (b) configuring the first fusion protein to bind proximally to the target nucleotide; (c) configuring a second fusion protein to bind proximally to a nucleotide opposite to the target nucleotide; and (d) administering an effective amount of the first and second fusion proteins, wherein, the first mitoTALE binds proximally to the target nucleotide and the second mitoTALE binds proximally to the nucleotide opposite the target nucleotide, and wherein the first portion of a DddA dimerizes with the second portion of a DddA, wherein the dimer has at least some activity native to full length DddA, and wherein the activity deaminates the target nucleotide.

In some embodiments, the disorder treated by the methods described herein is a genetic disorder. In some embodiments, the genetic disorder is a mitochondrial genetic disorder. In some embodiments, the mitochondrial disorder is selected from: MELAS/Leigh syndrome and Leber's hereditary optic neuropathy. In some embodiments, the mitochondrial disorder is MELAS/Leigh syndrome. In some embodiments, the mitochondrial disorder is Leber's hereditary optic neuropathy.

In some embodiments, the subject treated by the methods described herein is a mammal. In some embodiments, the mammal is human.

In another aspect, the disclosure relates to a kit comprising the first and/or second fusion proteins as disclosed herein, the pair of fusion proteins as disclosed herein, the dimer as disclosed herein, the nucleic acids as disclosed herein, the vector as disclosed herein, the composition as disclosed herein, and/or the isolated cell as disclosed herein. The vector may be an AAV vector (e.g., AAV1, AAV2, AAV3, AAV4, AAV5, AAV6, AAV7, AAV8, AAV9, or other serotype), a lentivirus vector, and may include one or more promoters that regulate the expression of the nucleotide sequences encoding the pair of fusion proteins.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

All previously described cytidine deaminases, including those used in base editing, operate on single-stranded DNA and thus when used for genome editing require unwinding of double-stranded DNA by macromolecules such as CRISPR-Cas9 complexed with a guide RNA. The difficulty of delivering guide RNAs into the mitochondria has thus far precluded base editing in mitochondrial DNA (mtDNA). The ability of DddA and the DddA variants described herein to deaminate double-stranded DNA raises the possibility of RNA-free precision base editing, rather than simple elimination of targeted mtDNA copies following double-strand DNA breaks. Split-DddA halves were engineered that are non-toxic and inactive until brought together on target DNA by adjacently bound programmable DNA-binding proteins. Fusions of the split-DddA halves, TALE array proteins, and uracil glycosylase inhibitor resulted in RNA-free DddA-derived cytosine base editors (DdCBEs) that catalyze C⋅G-to-T⋅A conversions efficiently and with high DNA sequence specificity and product purity at targeted sites within mtDNA in human cells.

DddA-mediated base editing was used to model a disease-associated mtDNA mutation in human cell lines, resulting in changes in rates of respiration and oxidative phosphorylation. CRISPR-free, DddA-mediated base editing enables precision editing of mtDNA, with important basic science and biomedical implications.

FIG. 1A is a schematic representation of a naturally occurring interbacterial toxin discovered by the inventors and catalyzes unprecedented deamination of cytidines within double-stranded DNA as a substrate. The protein is referred to as a double-stranded DNA deaminase, which is referred to herein as a “DddA.” The inventors are believed to be the first to identify such a deaminase. However, in its naturally occurring form, the inventors discovered that DddA is toxic to cells. The inventors have conceived of the idea of using the DddA in the context of base editing to deaminate a nucleobase at a target edit site.

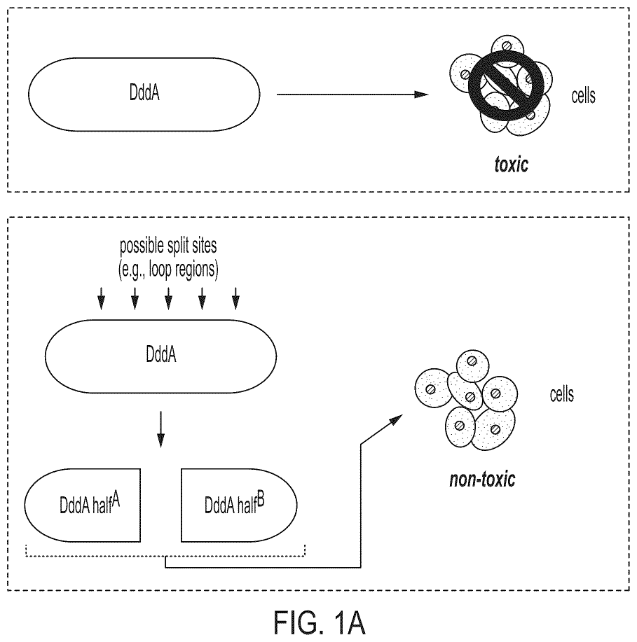

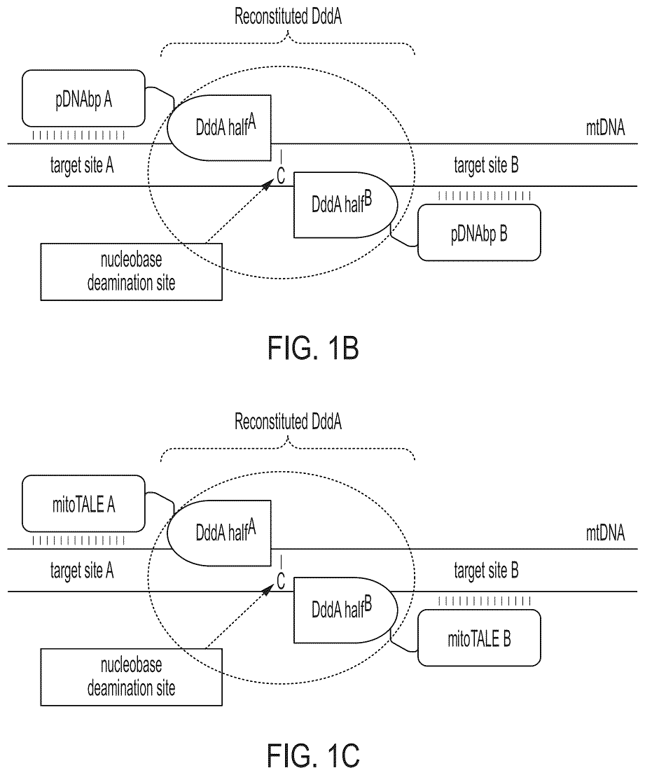

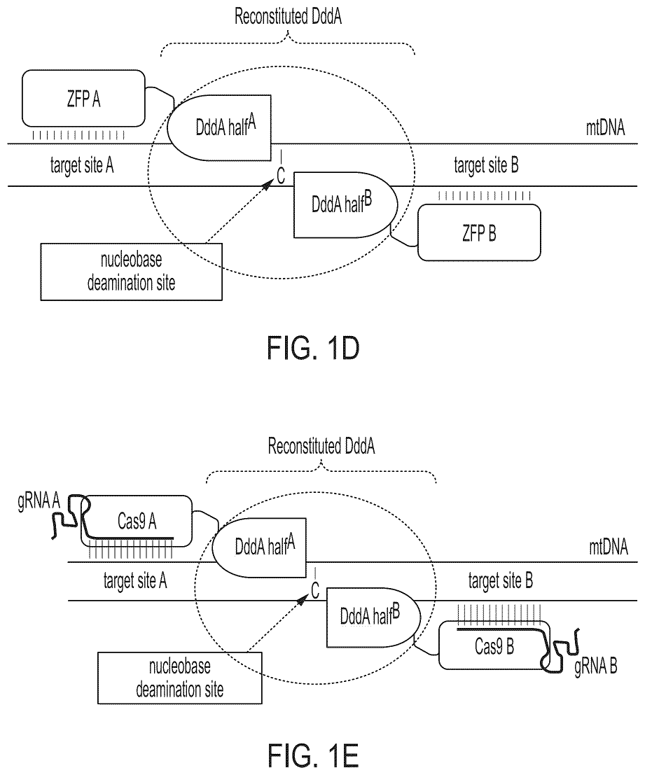

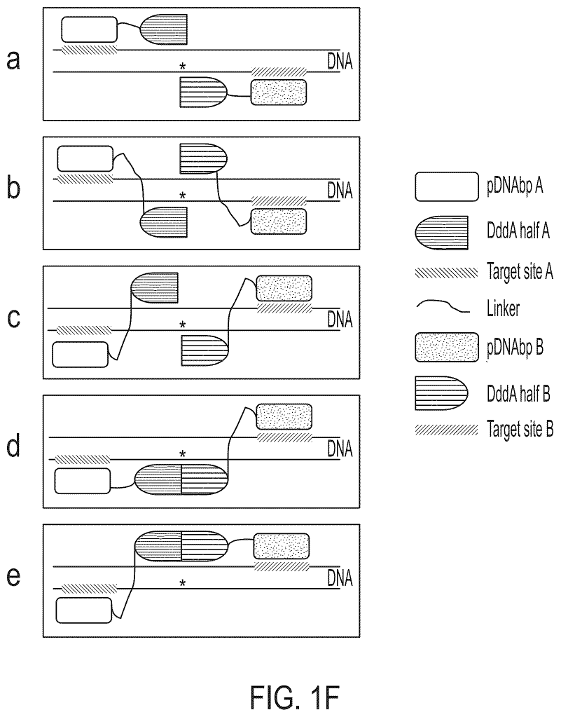

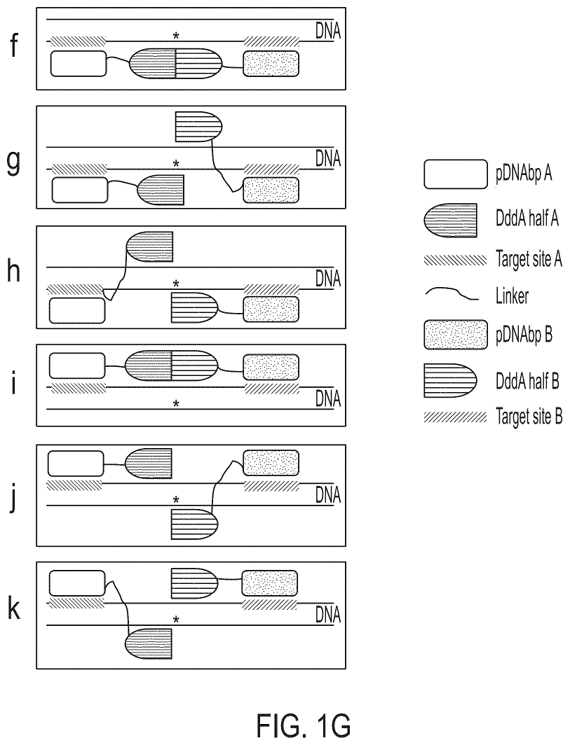

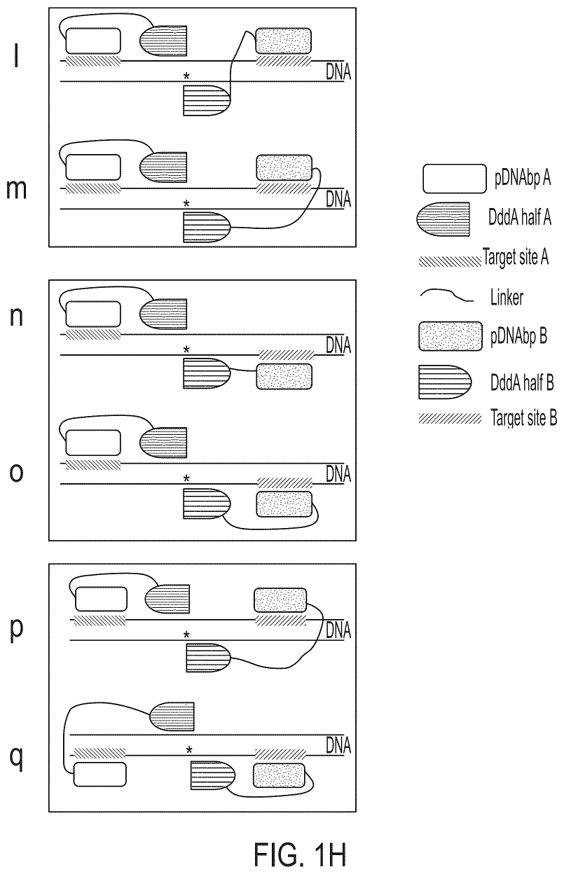

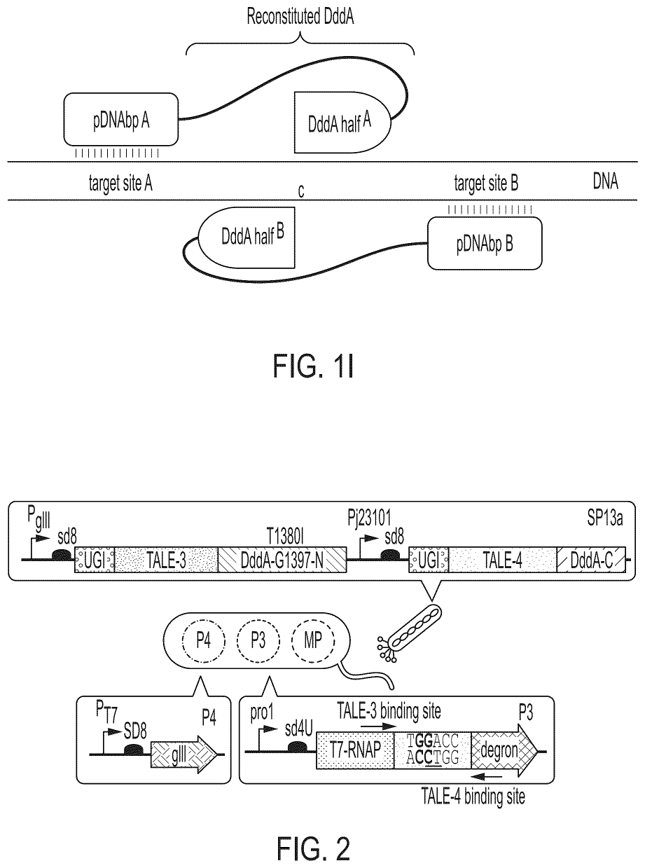

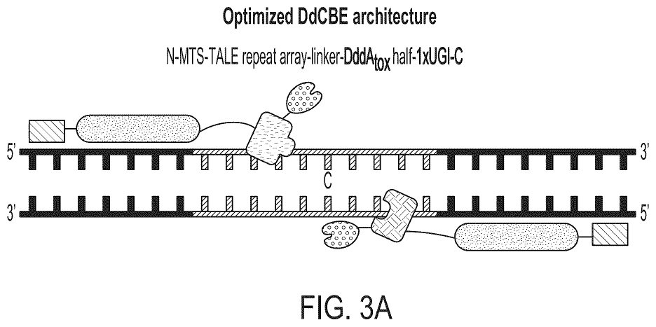

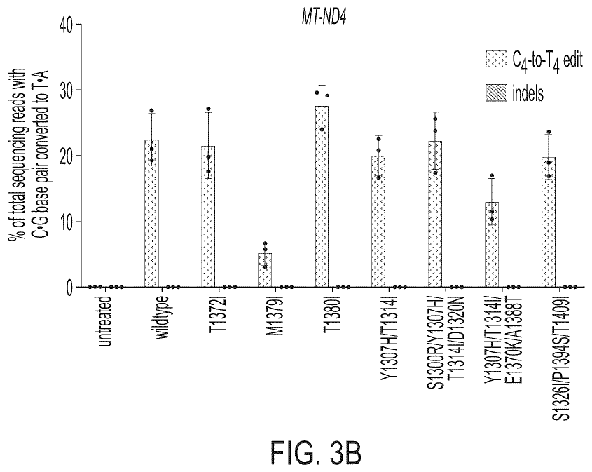

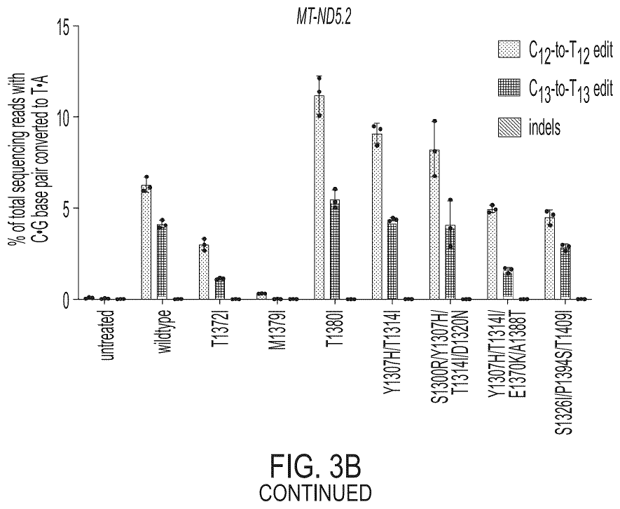

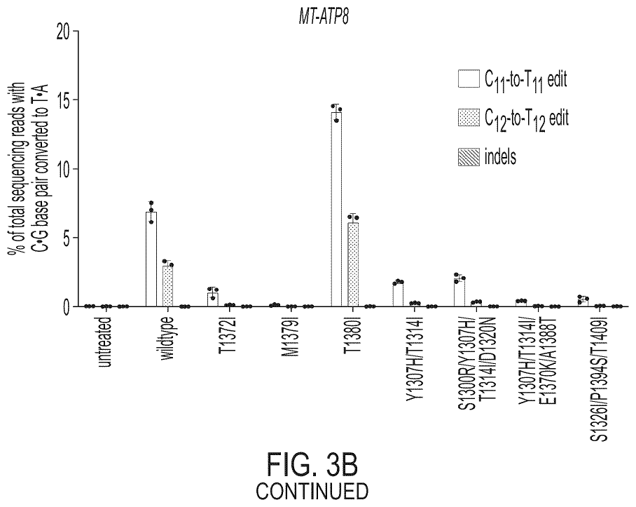

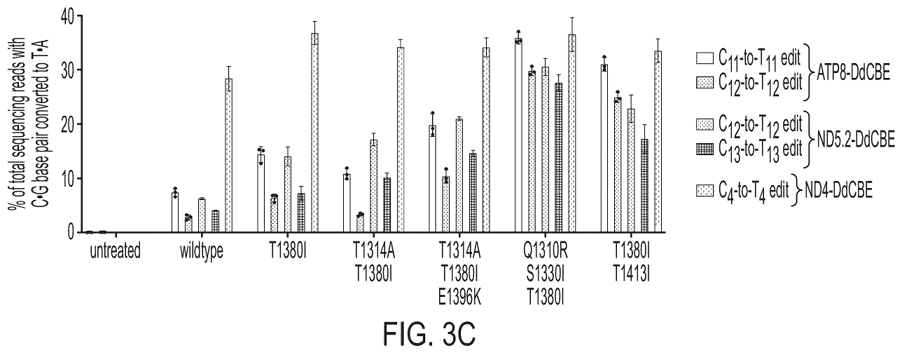

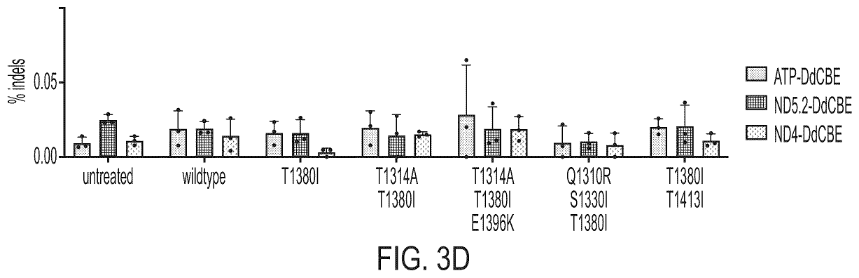

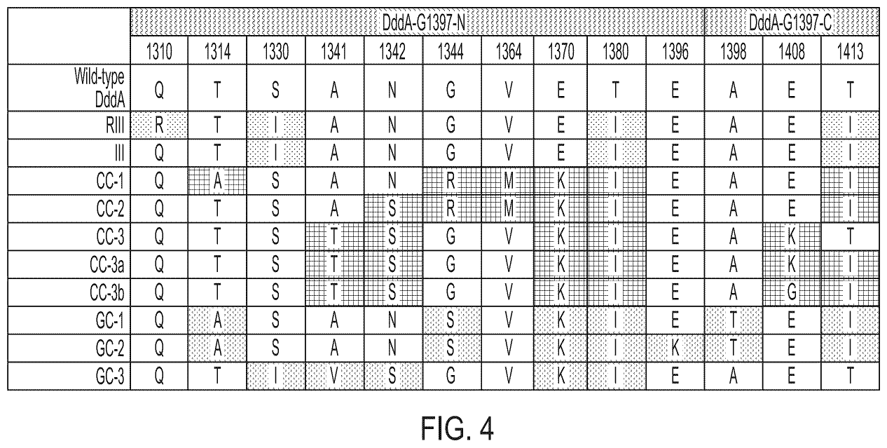

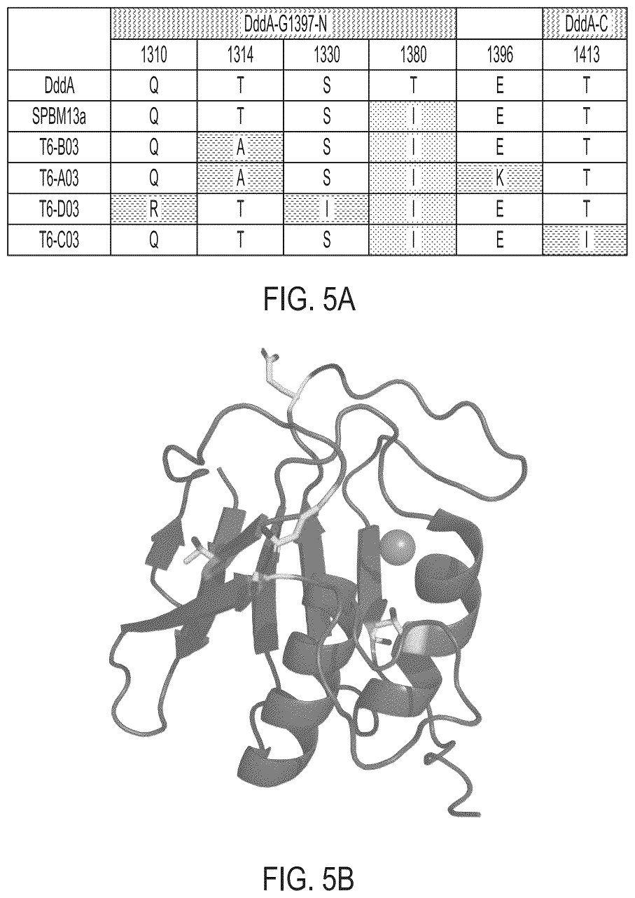

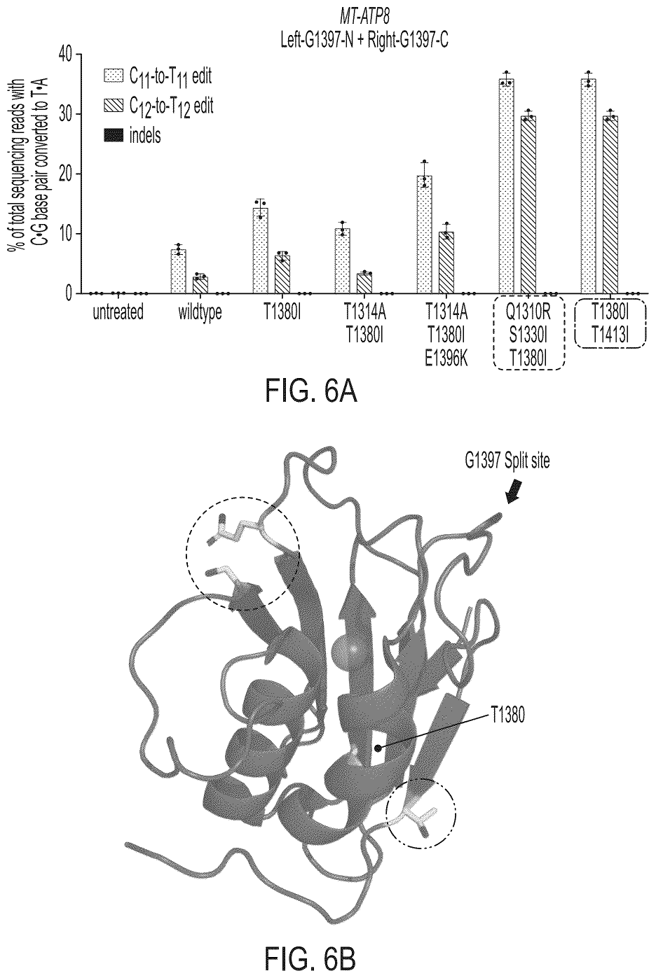

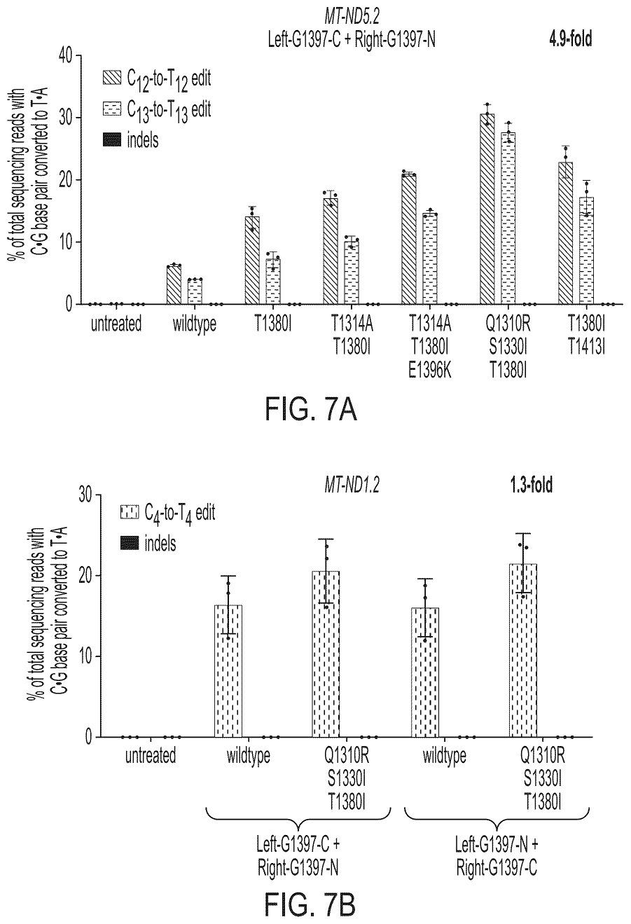

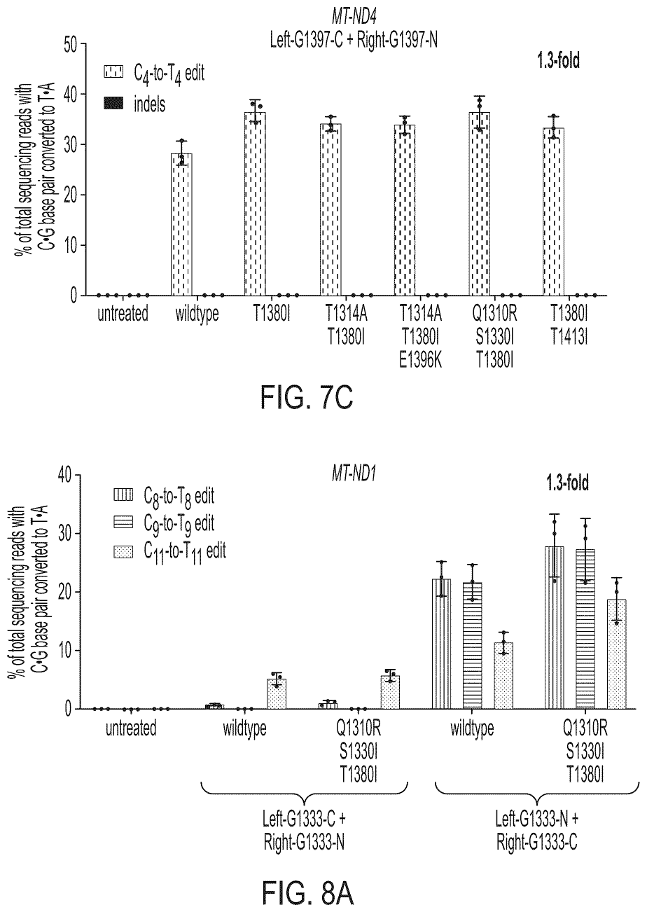

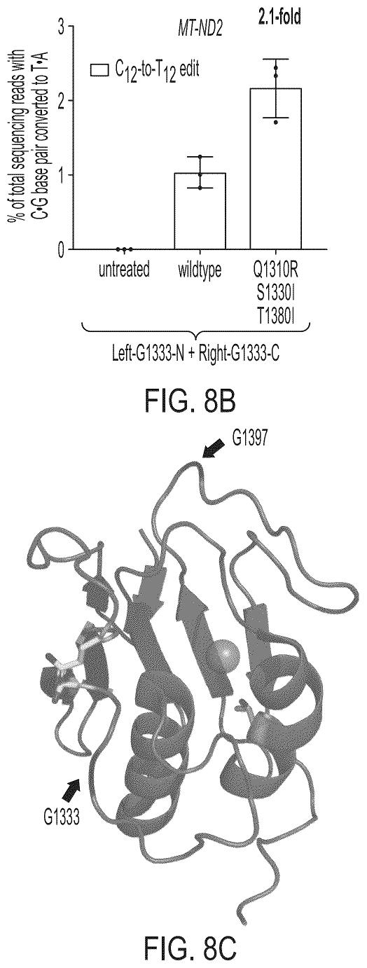

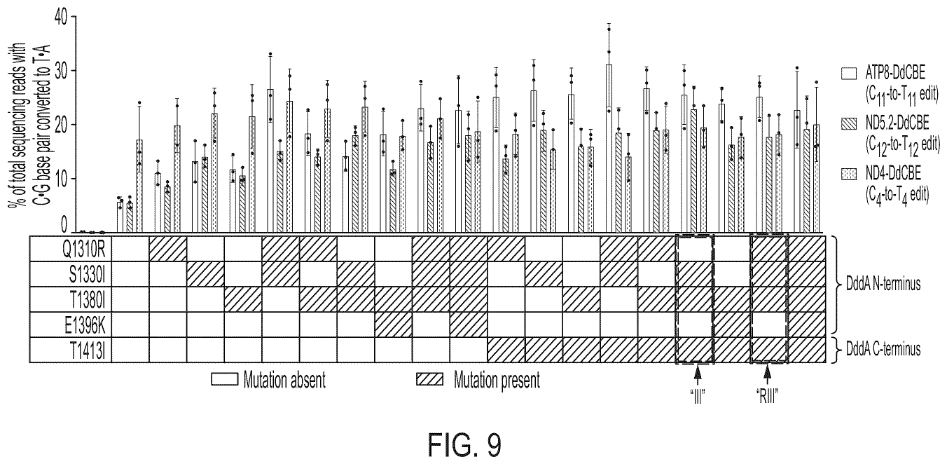

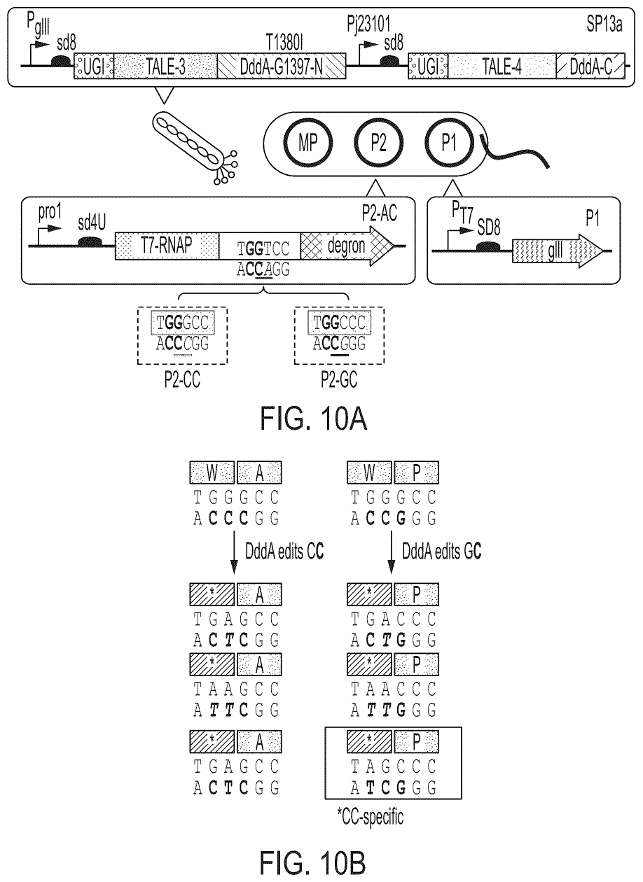

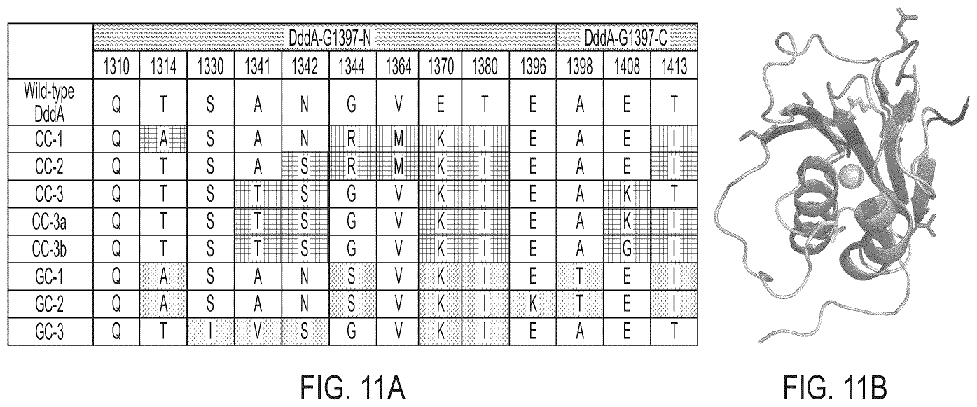

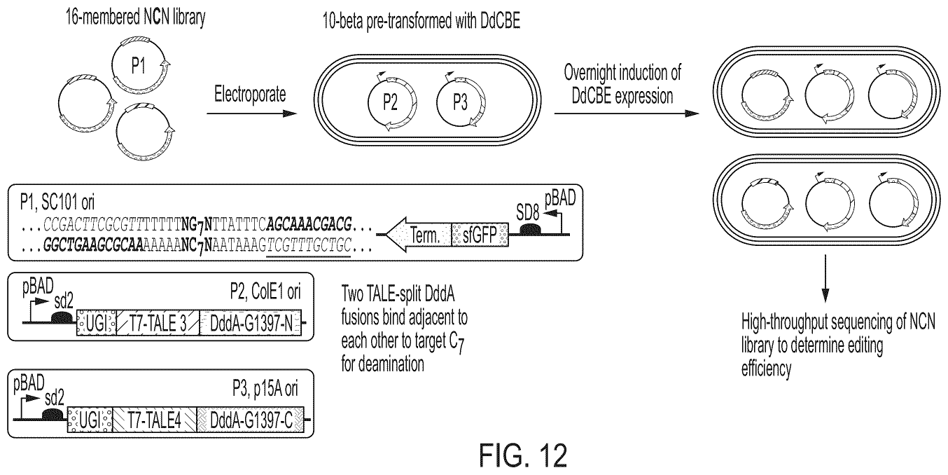

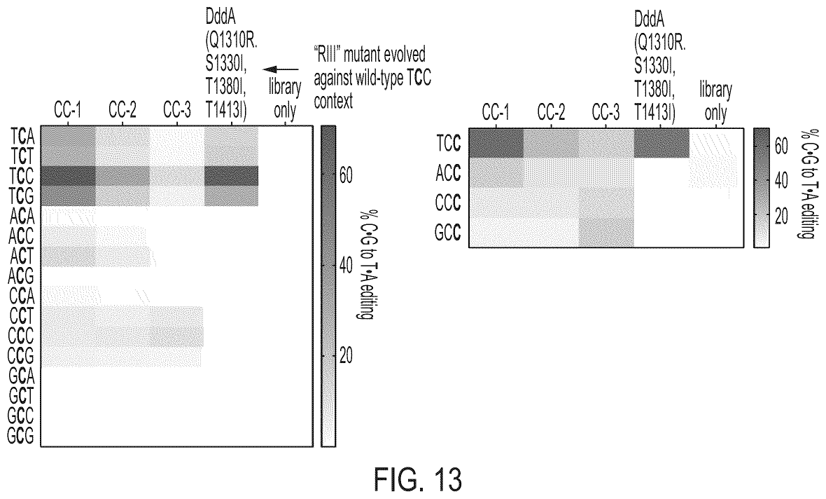

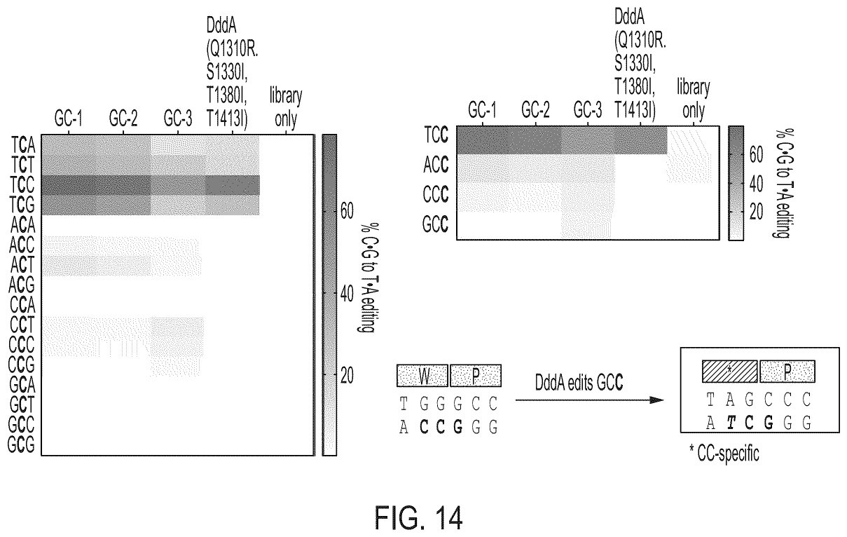

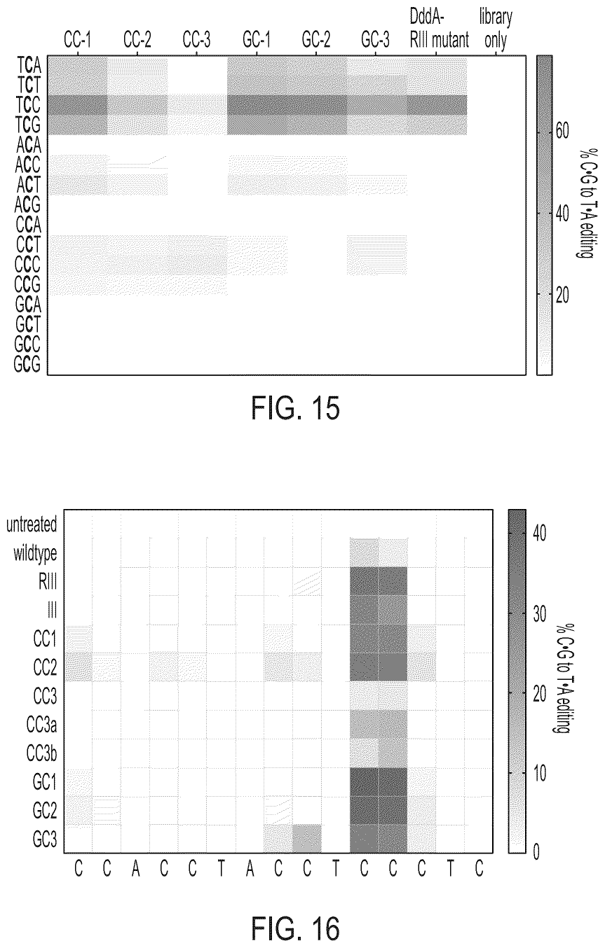

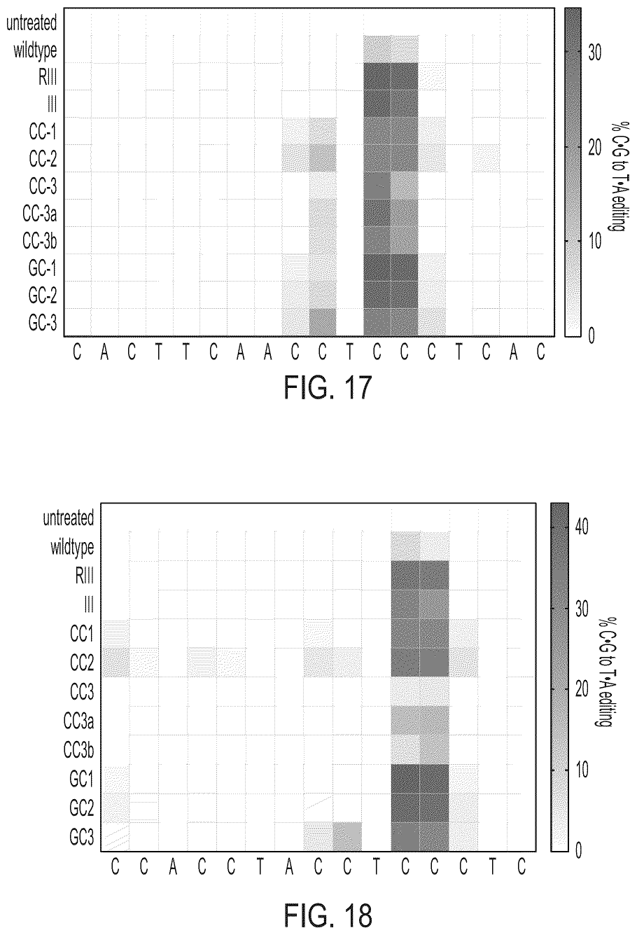

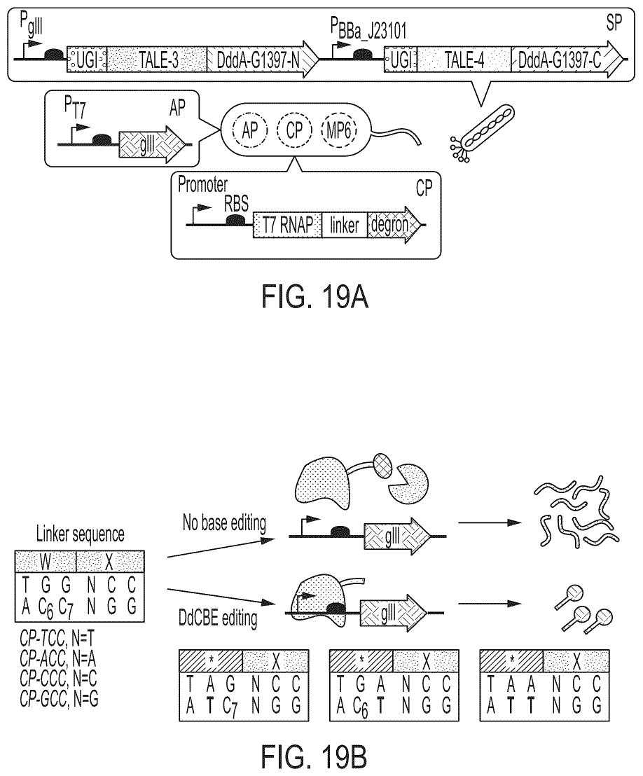

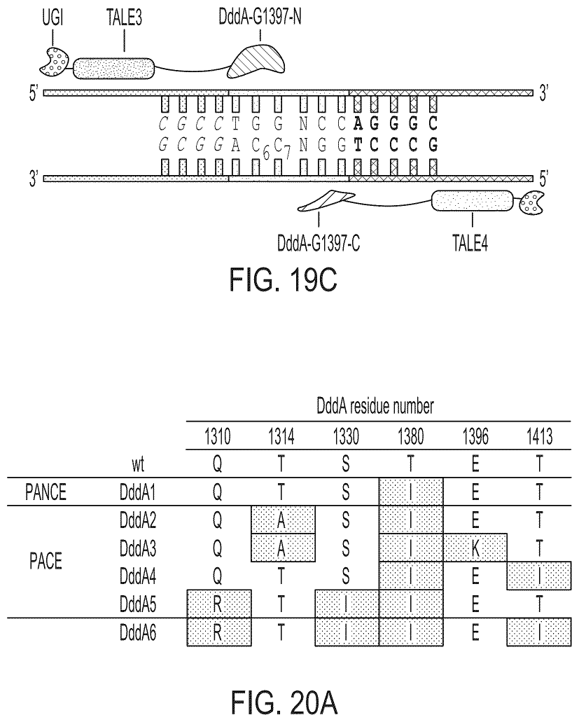

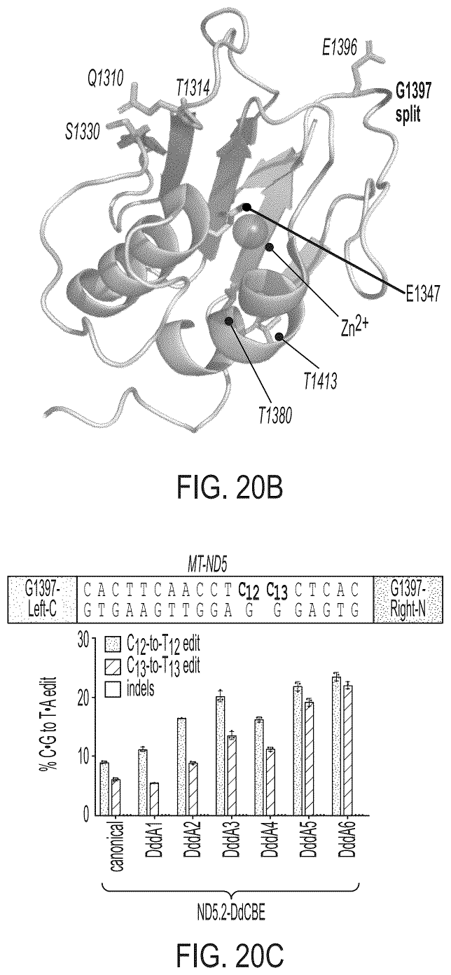

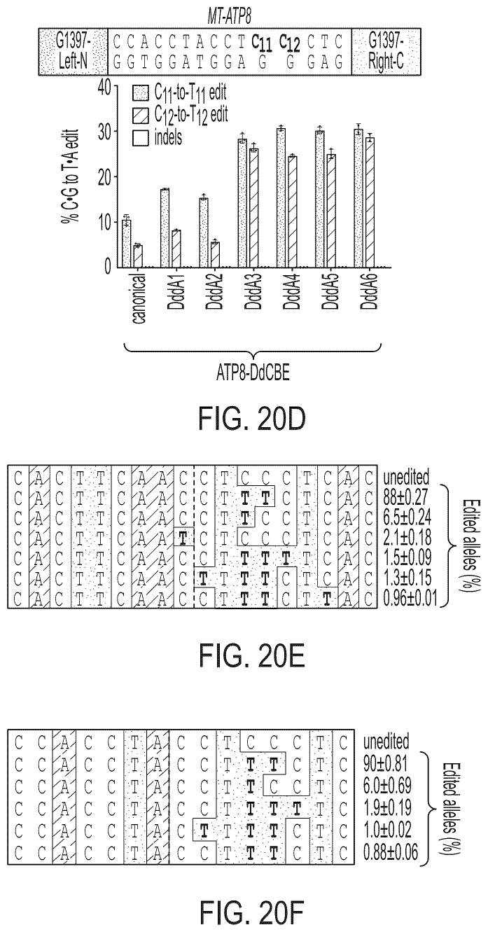

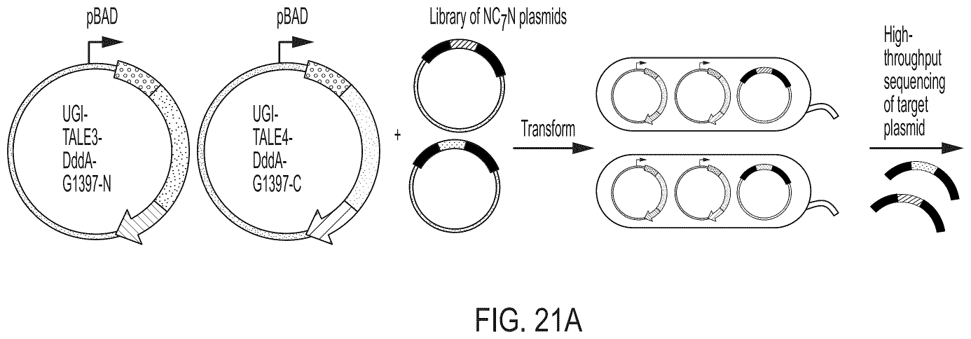

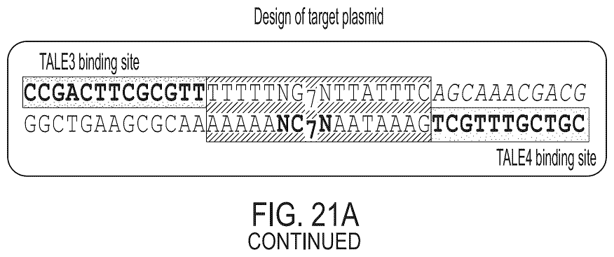

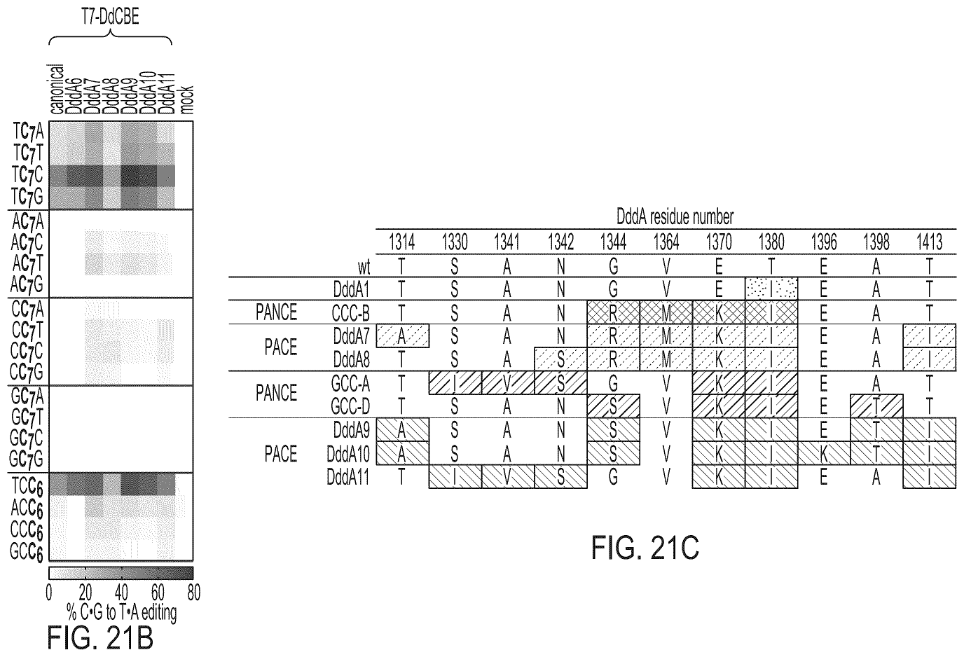

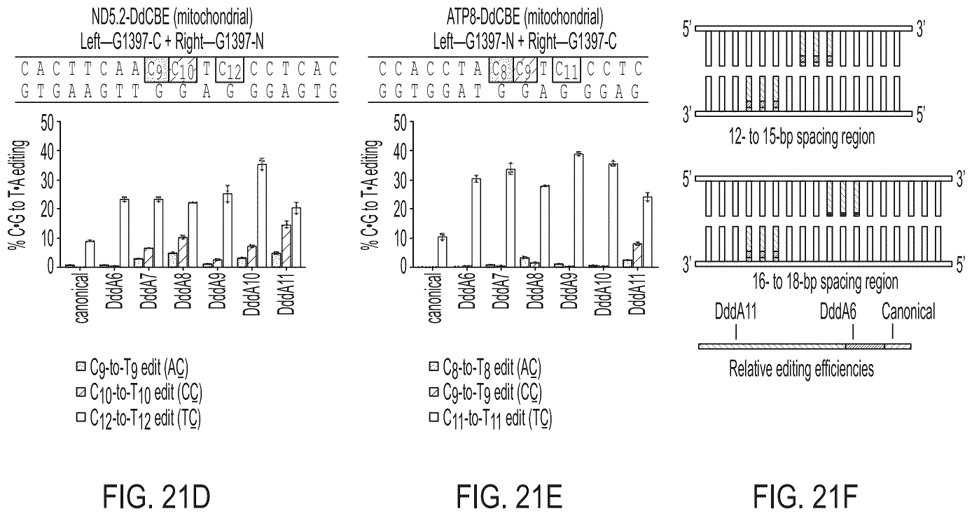

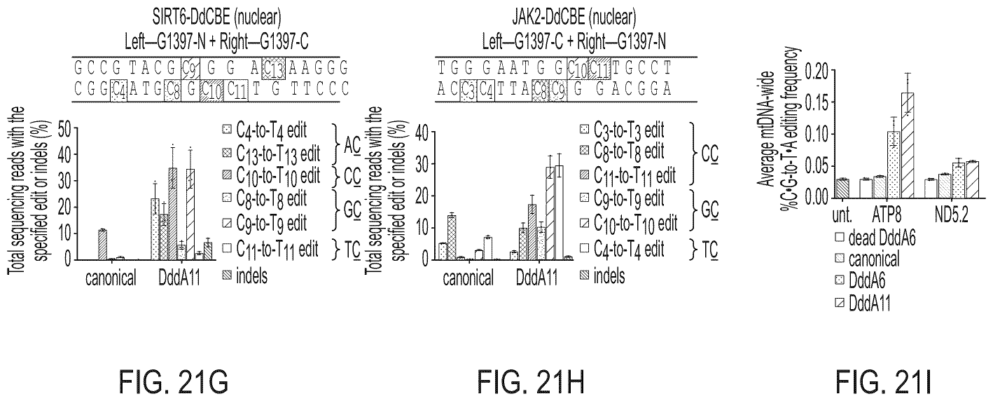

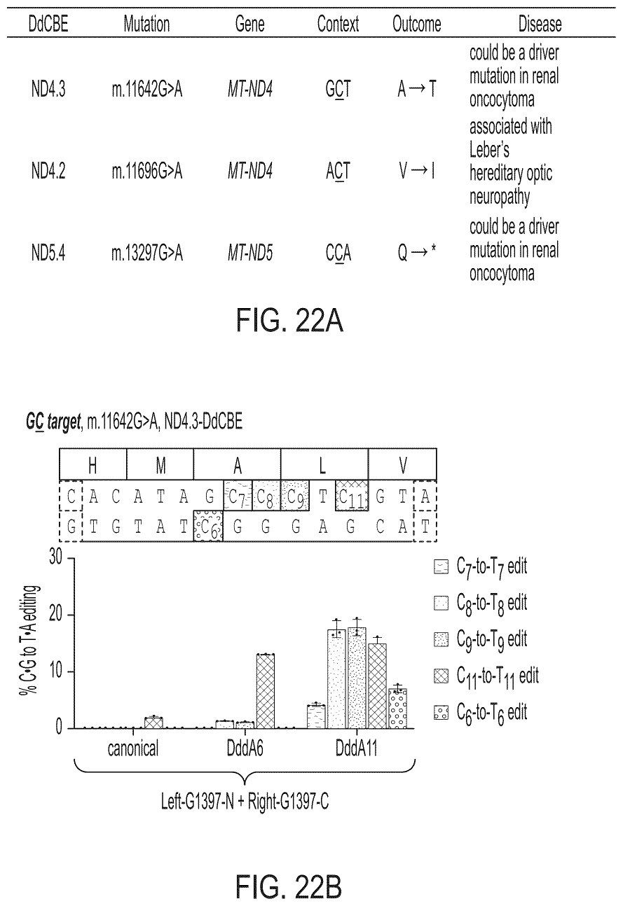

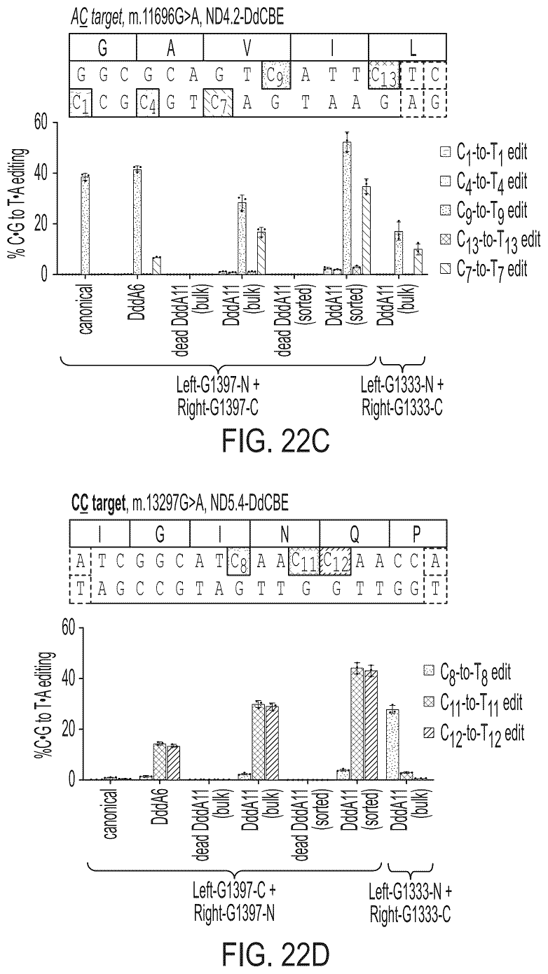

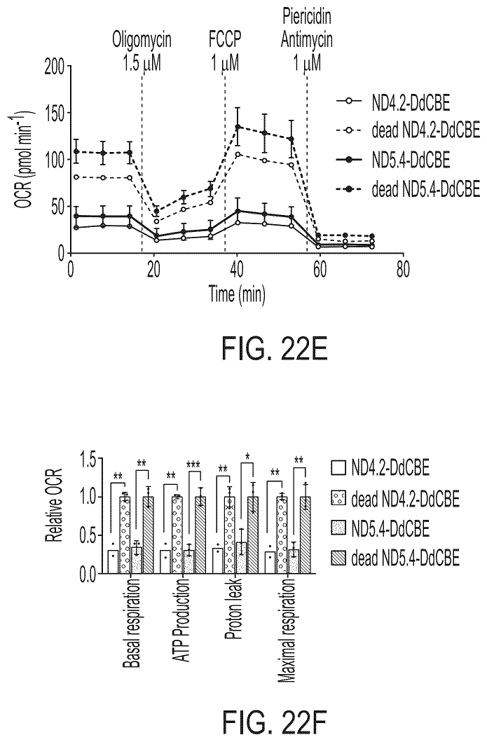

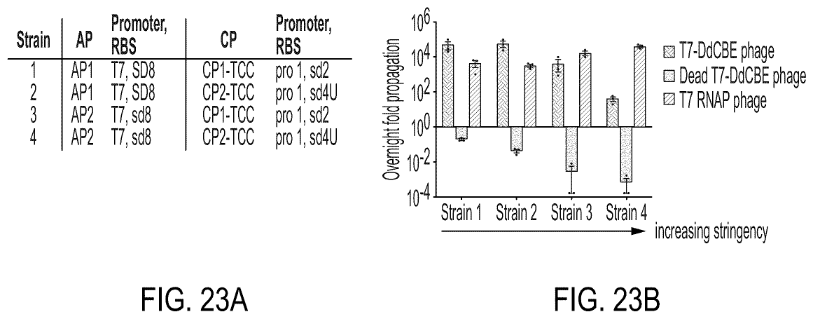

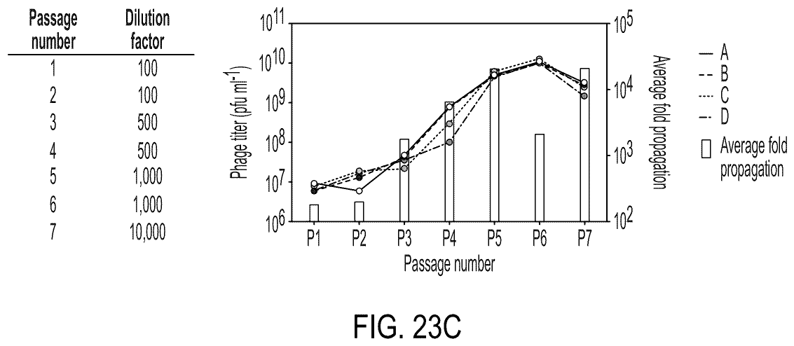

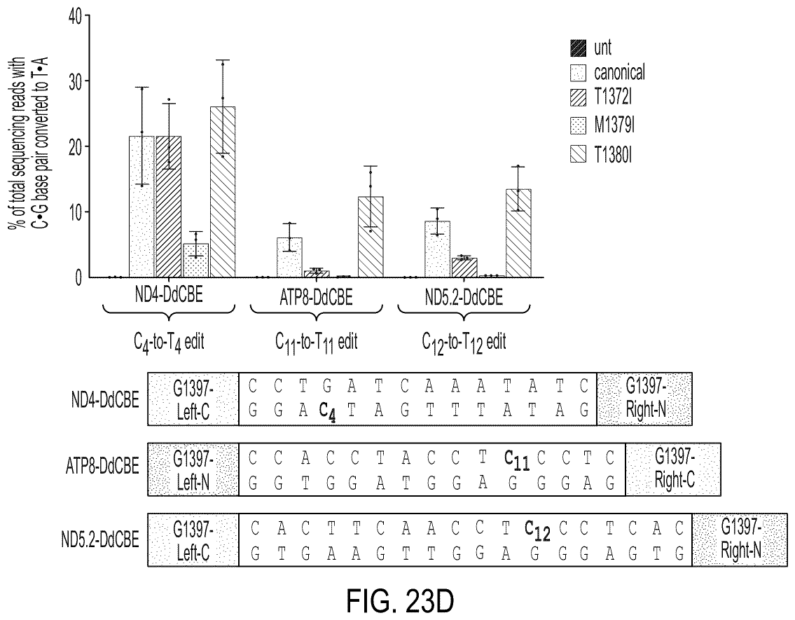

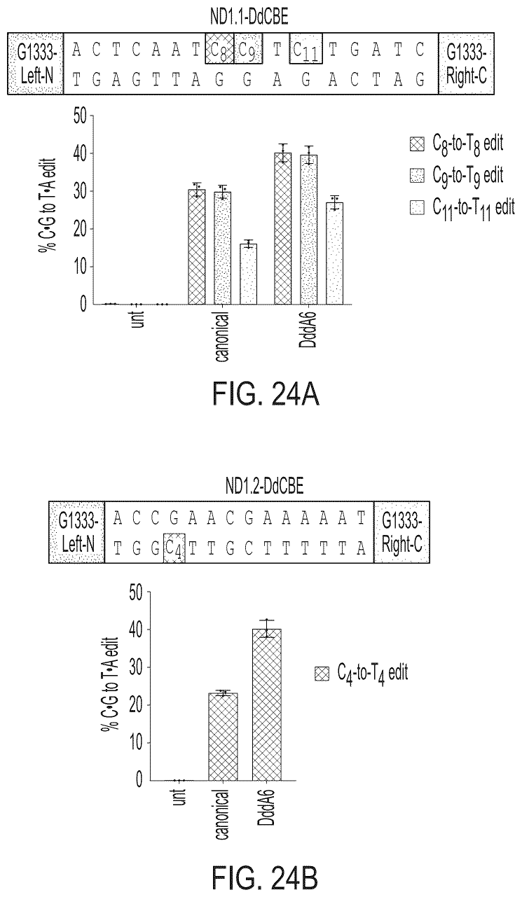

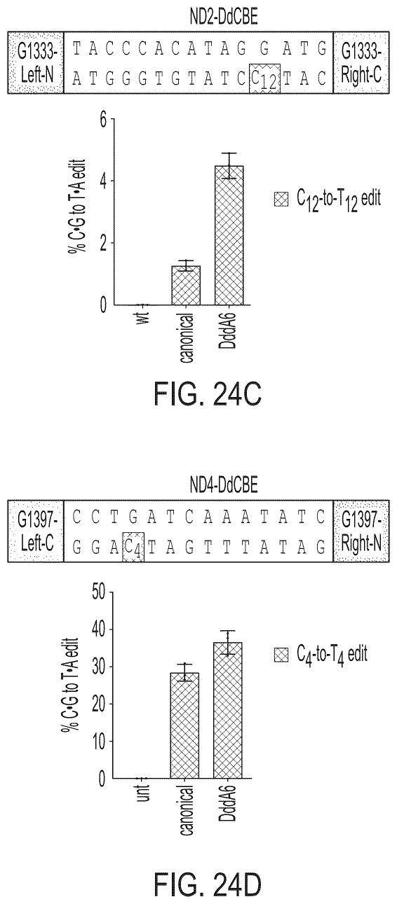

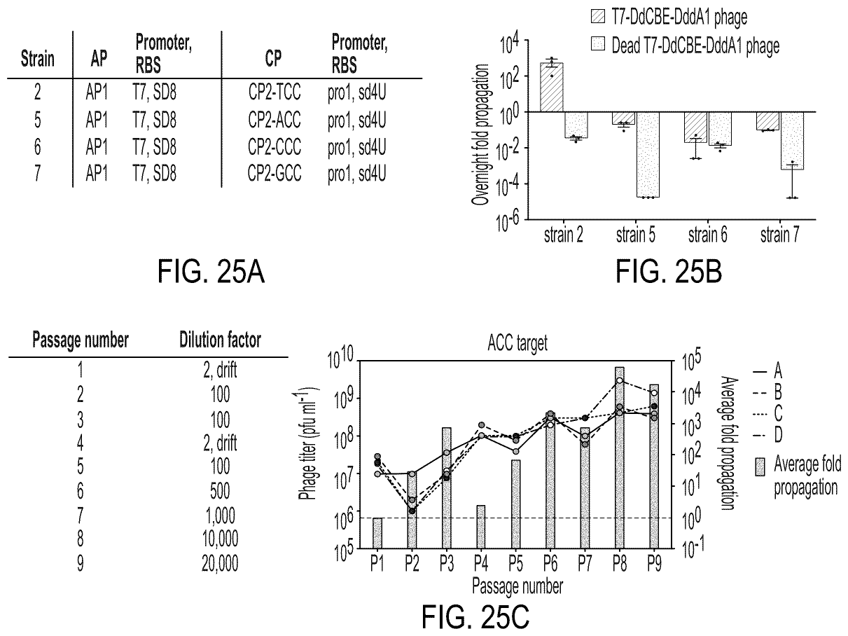

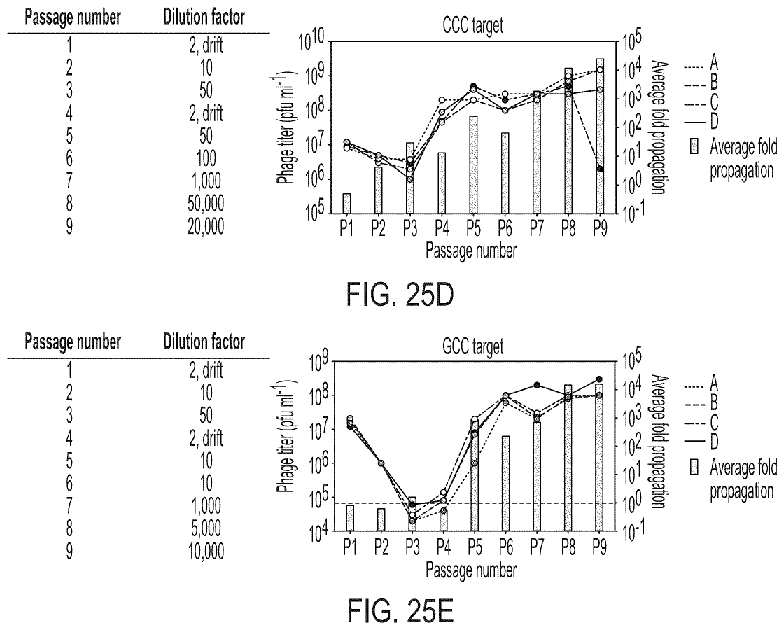

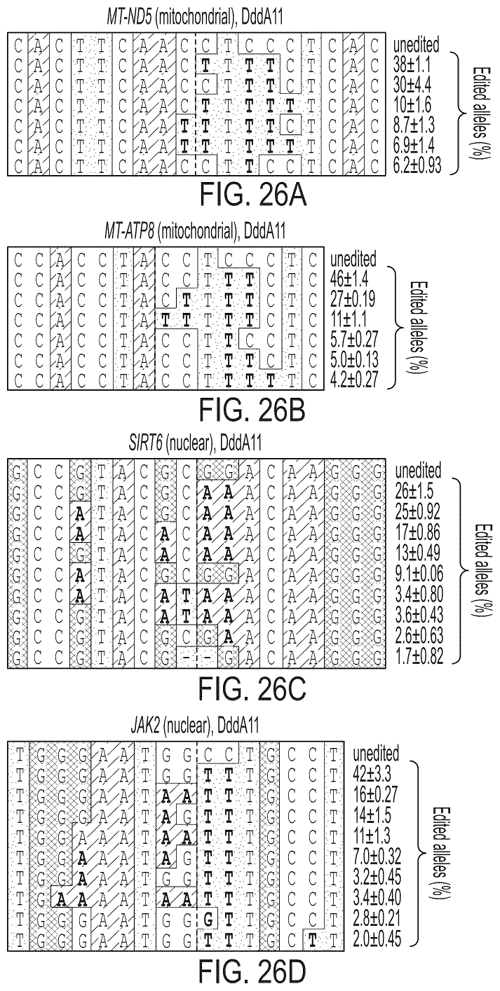

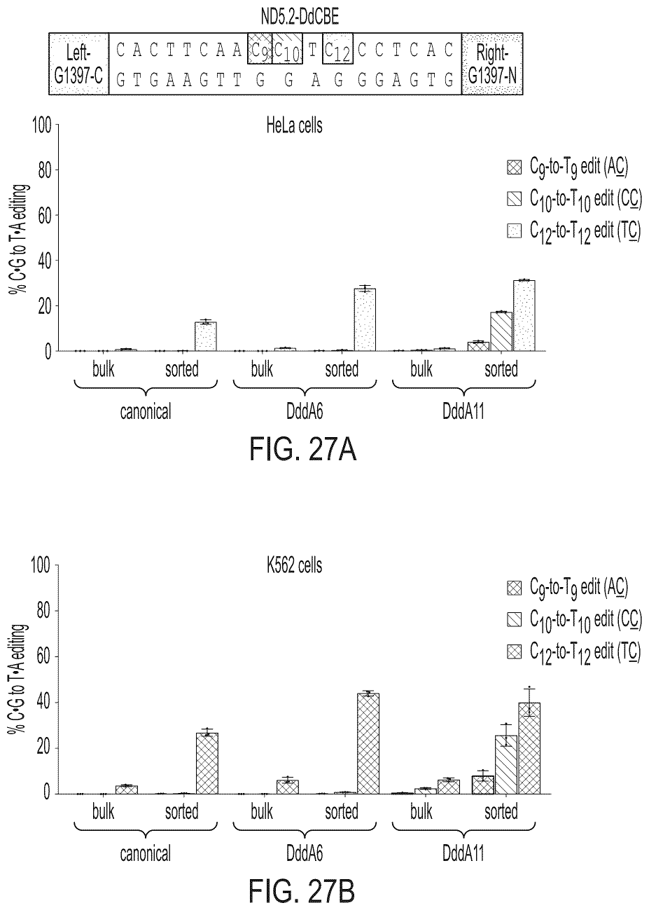

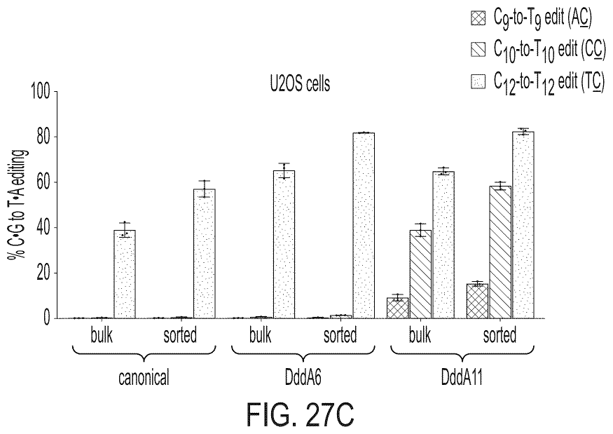

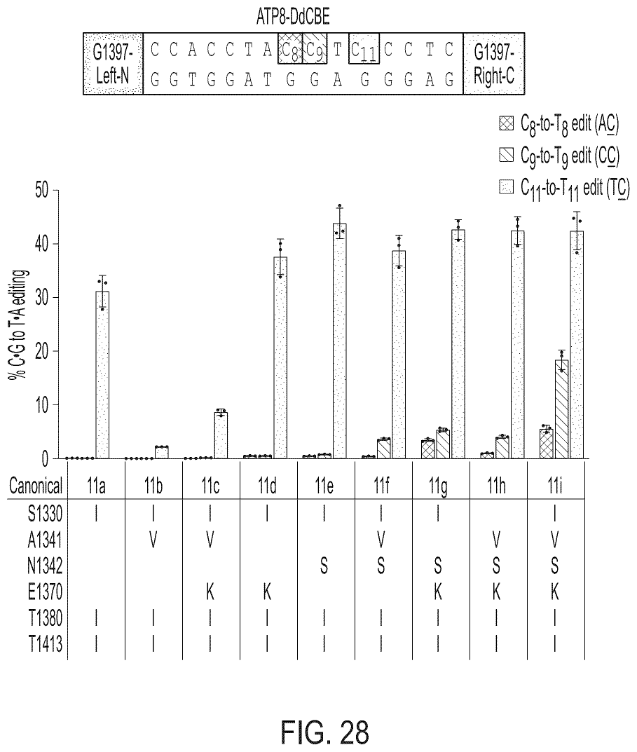

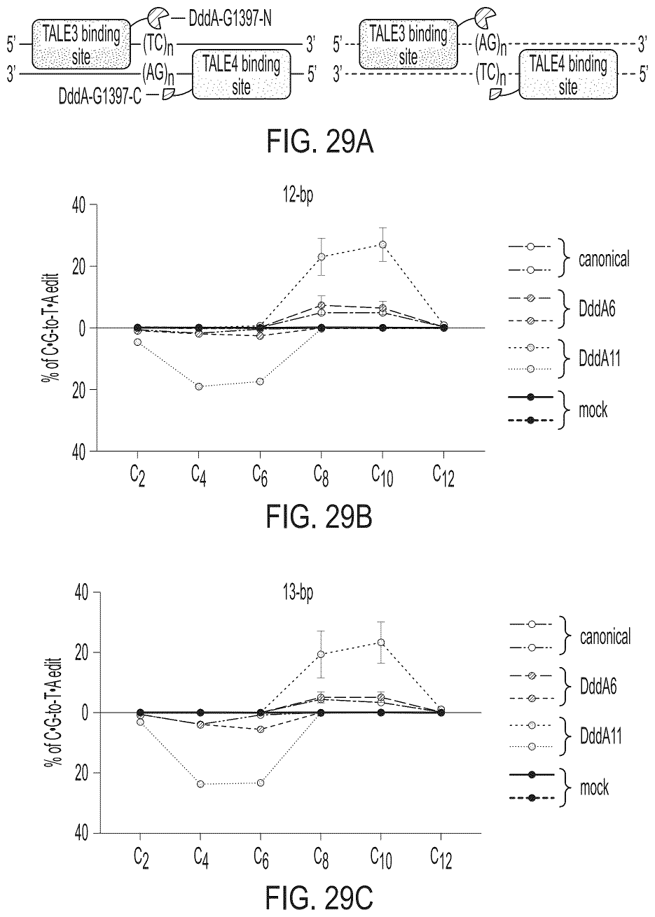

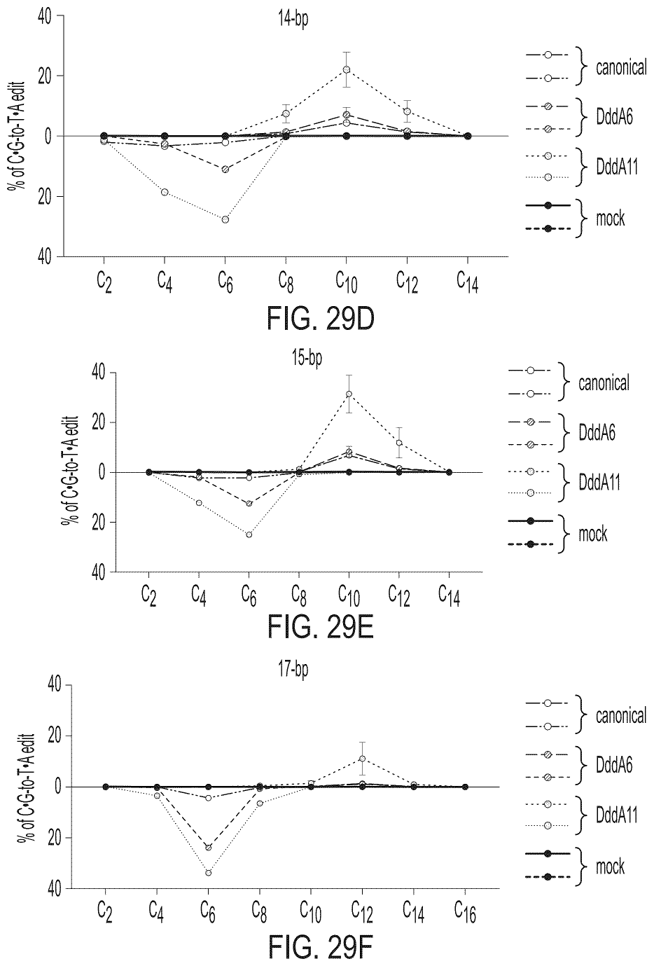

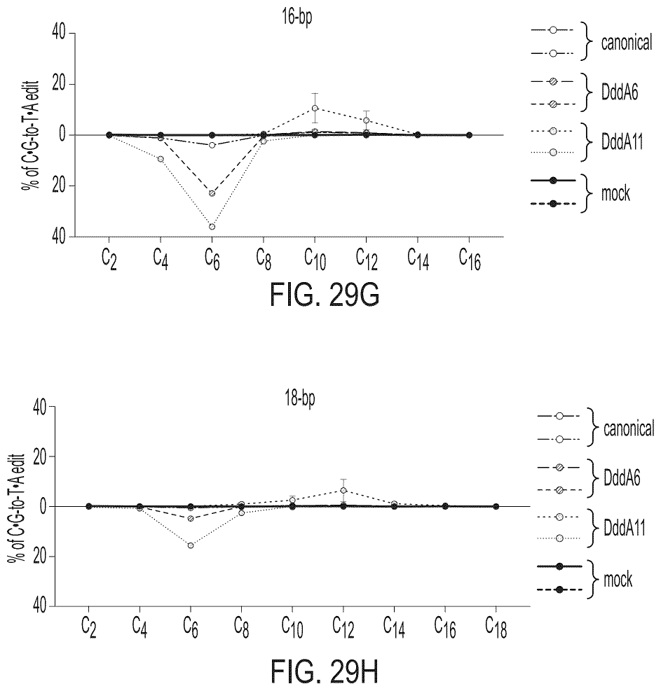

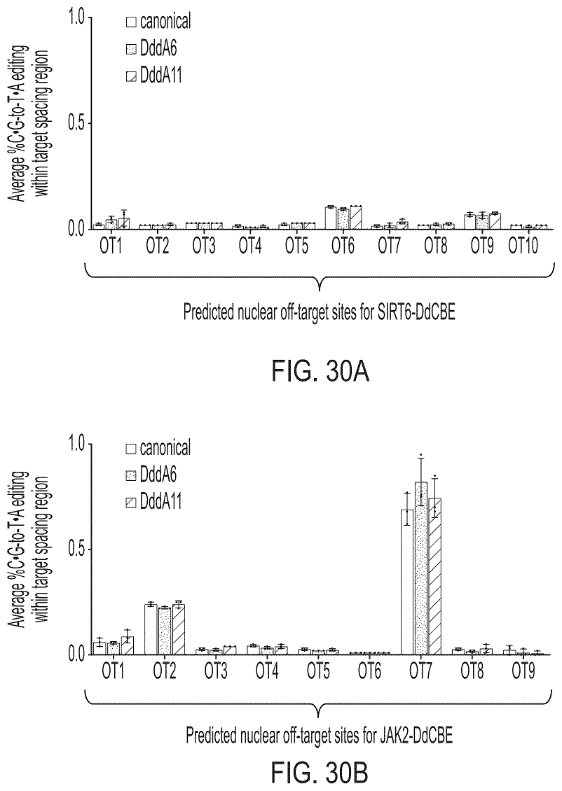

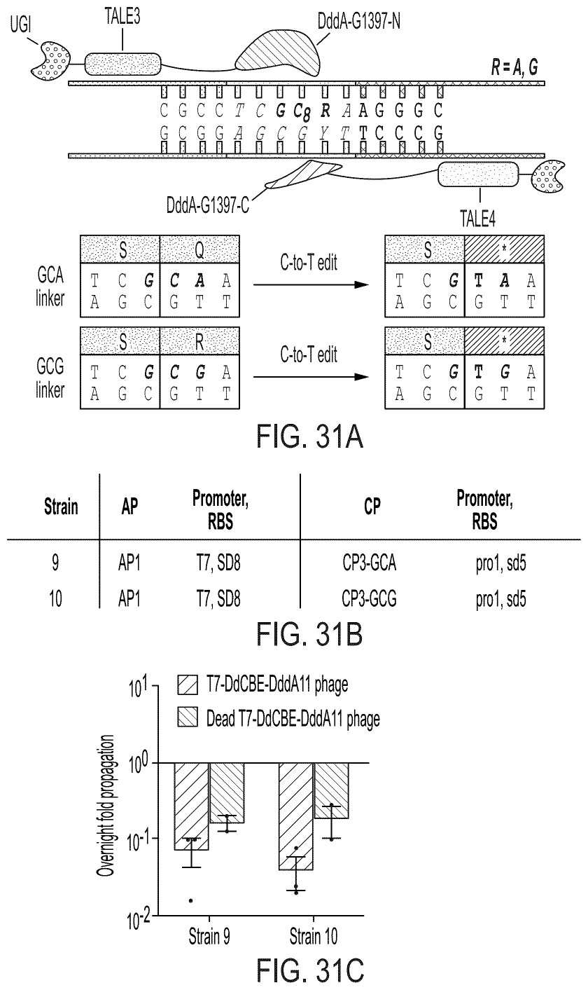

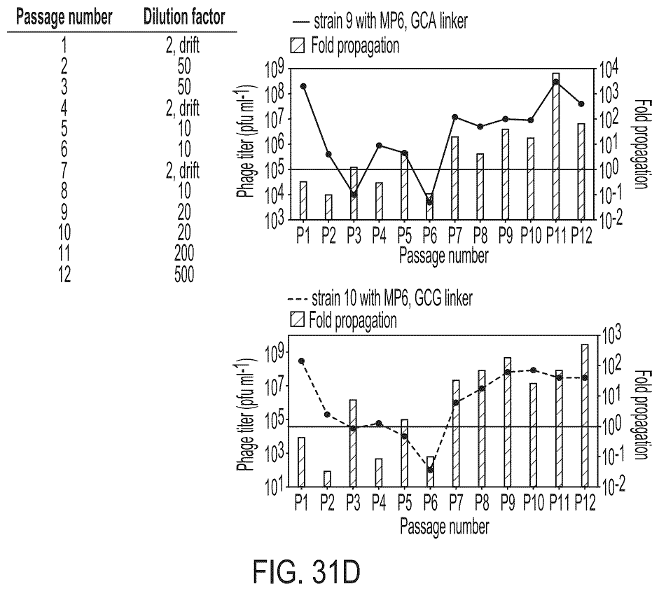

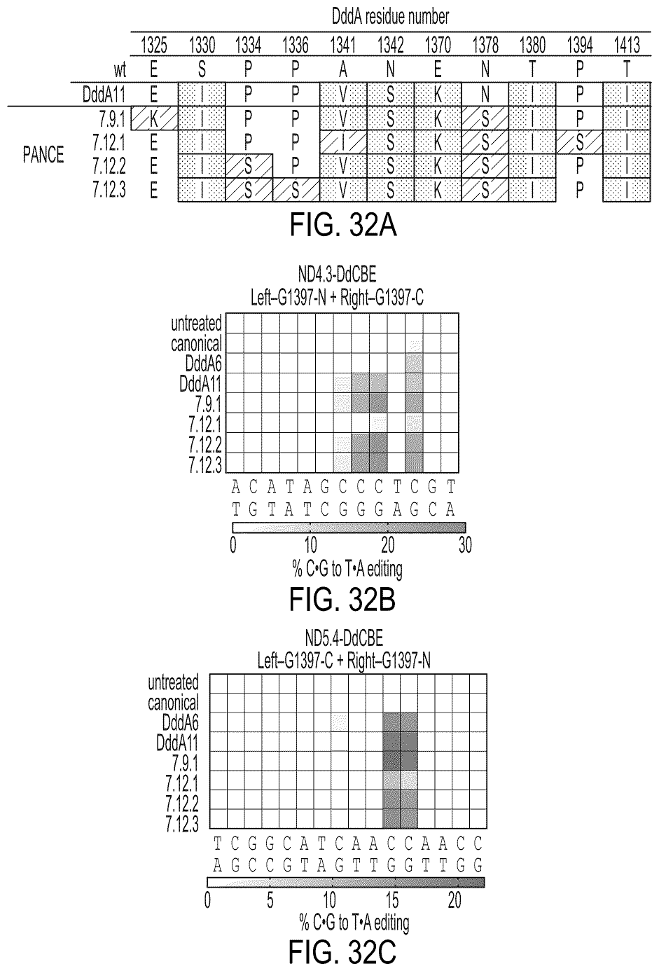

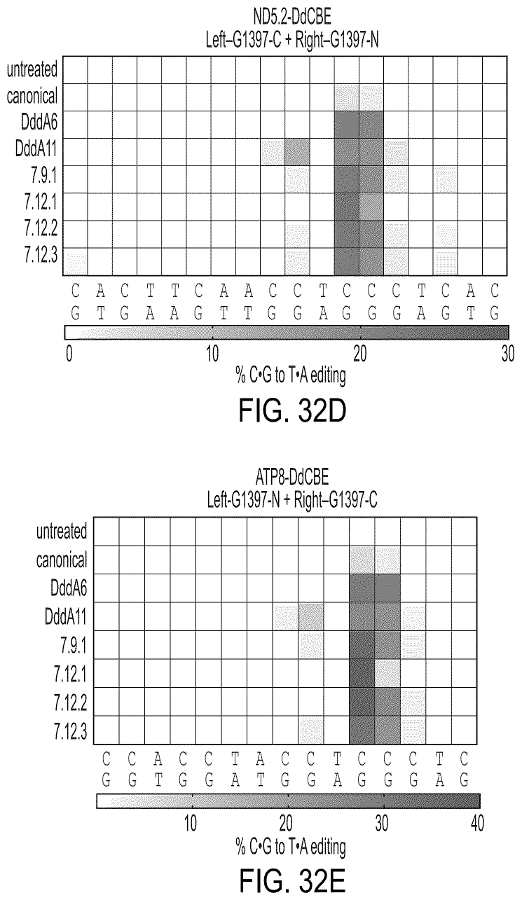

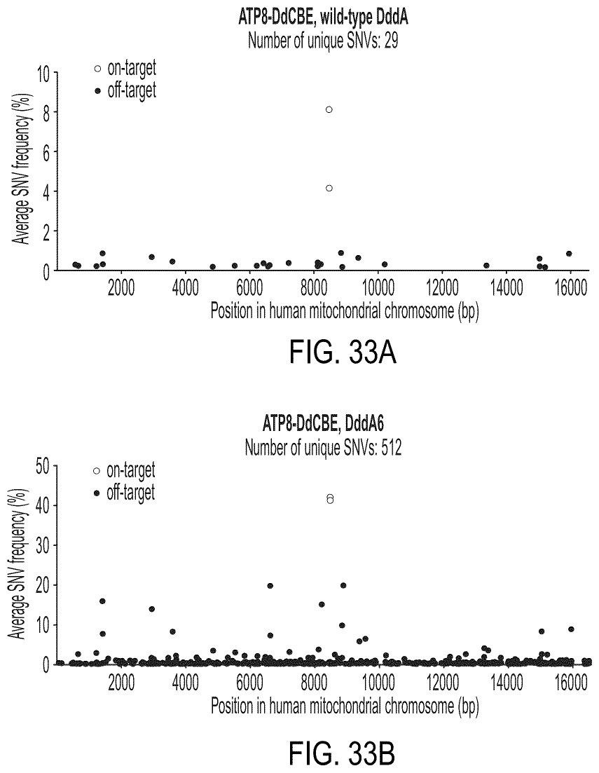

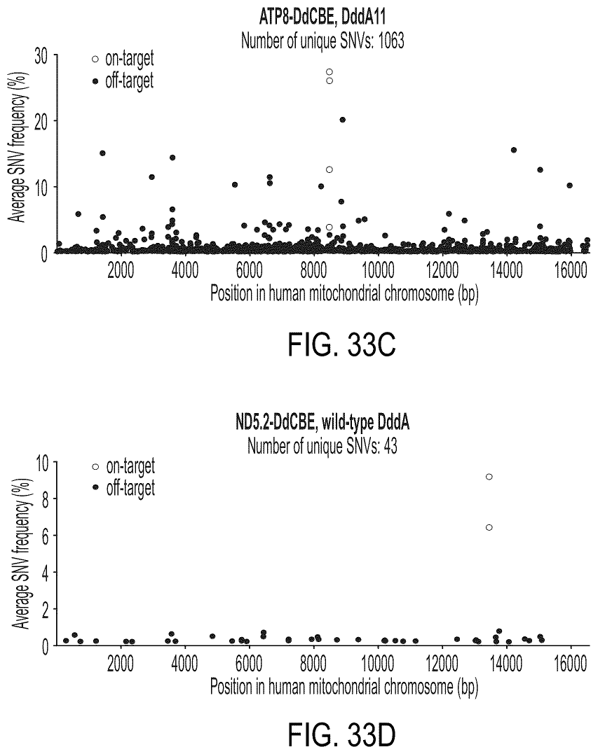

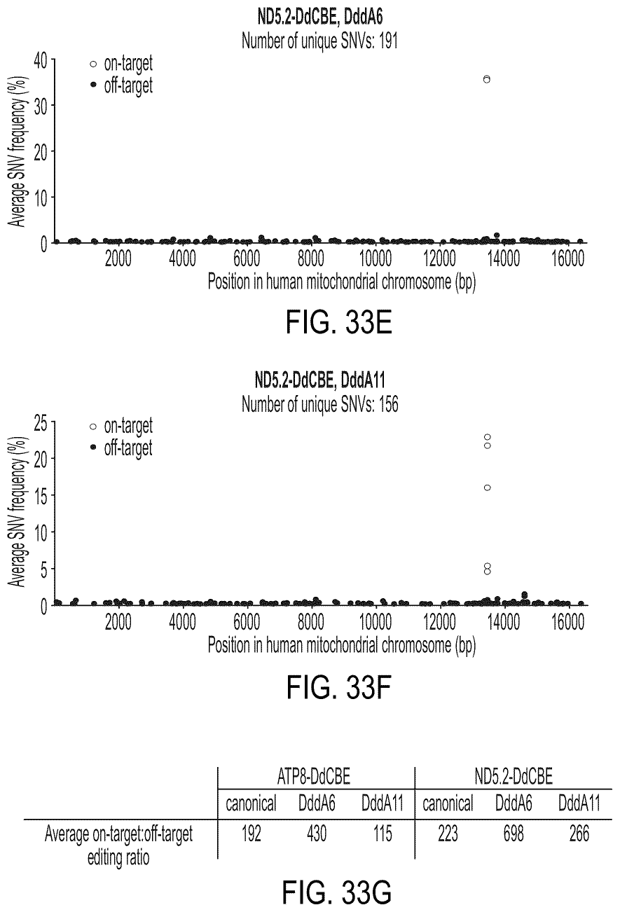

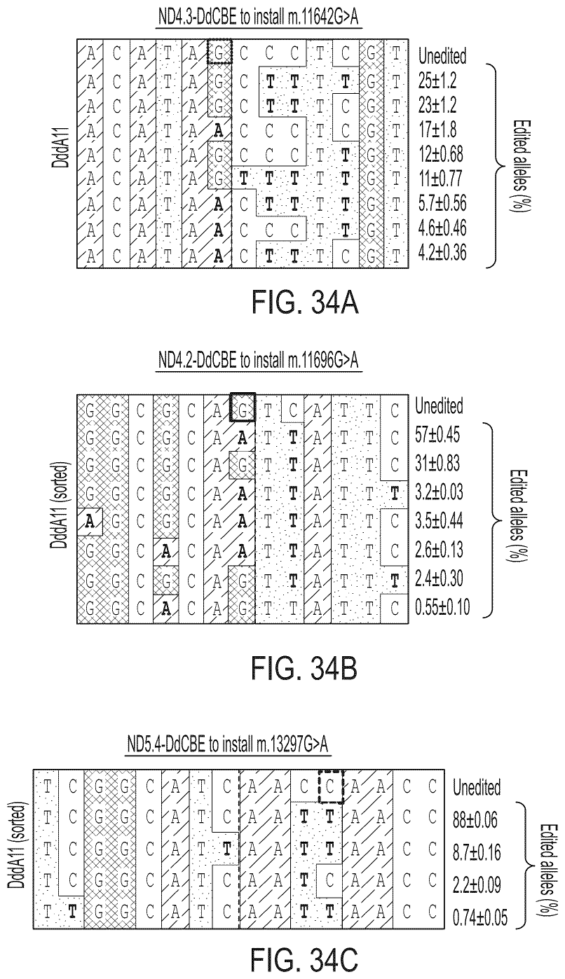

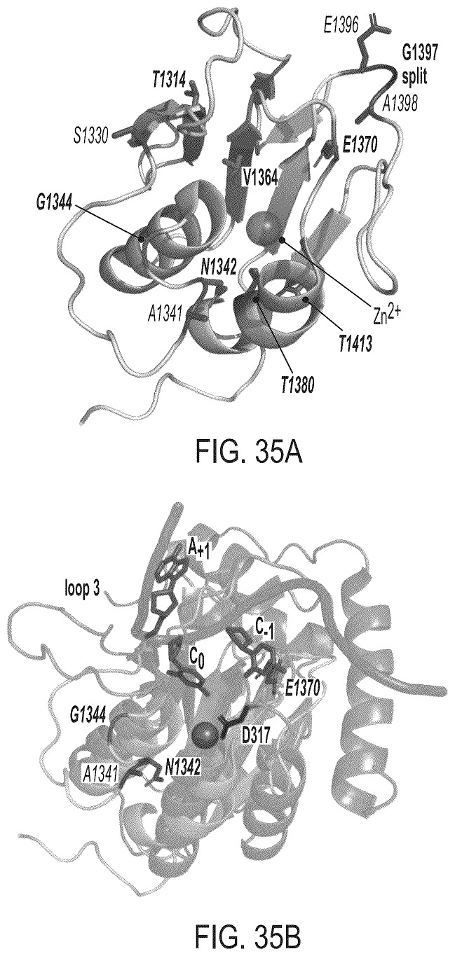

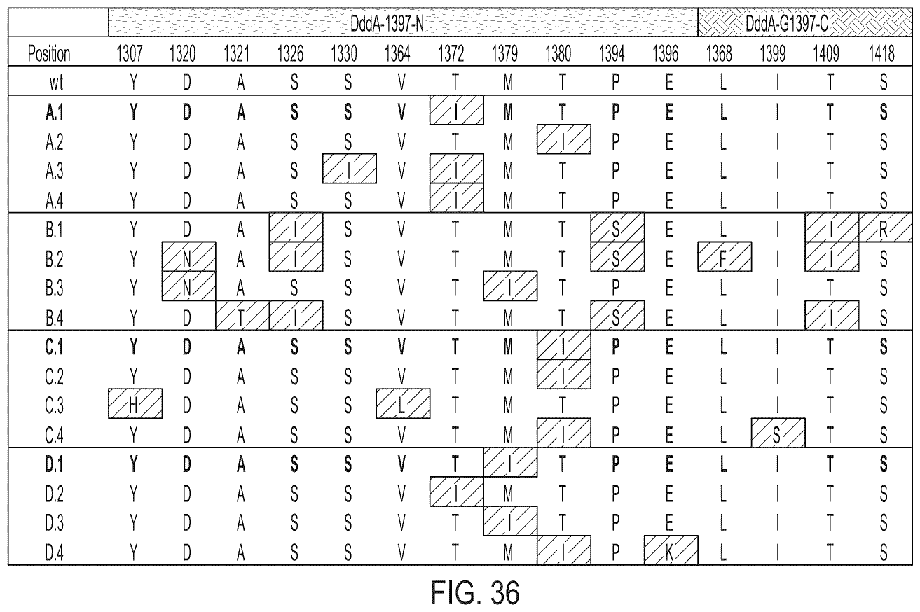

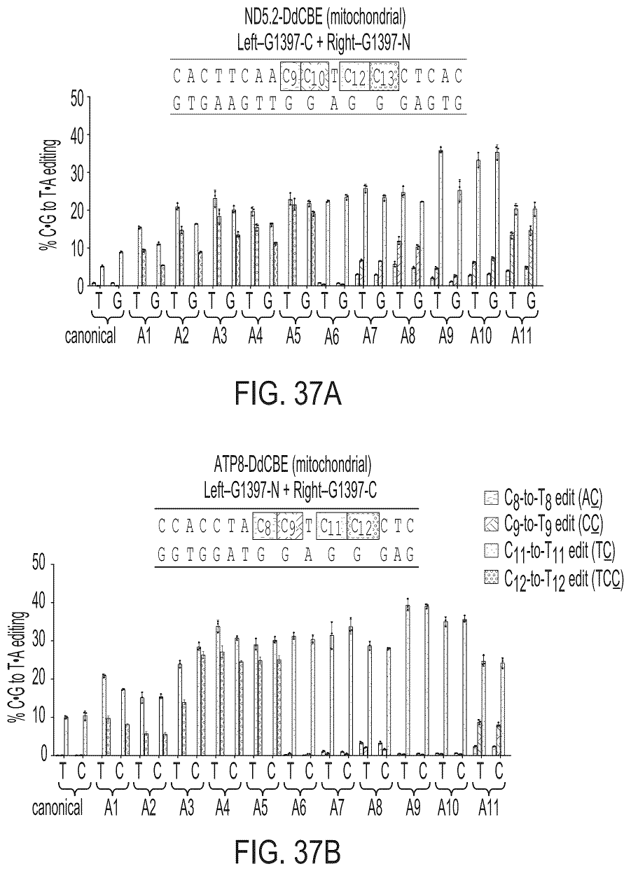

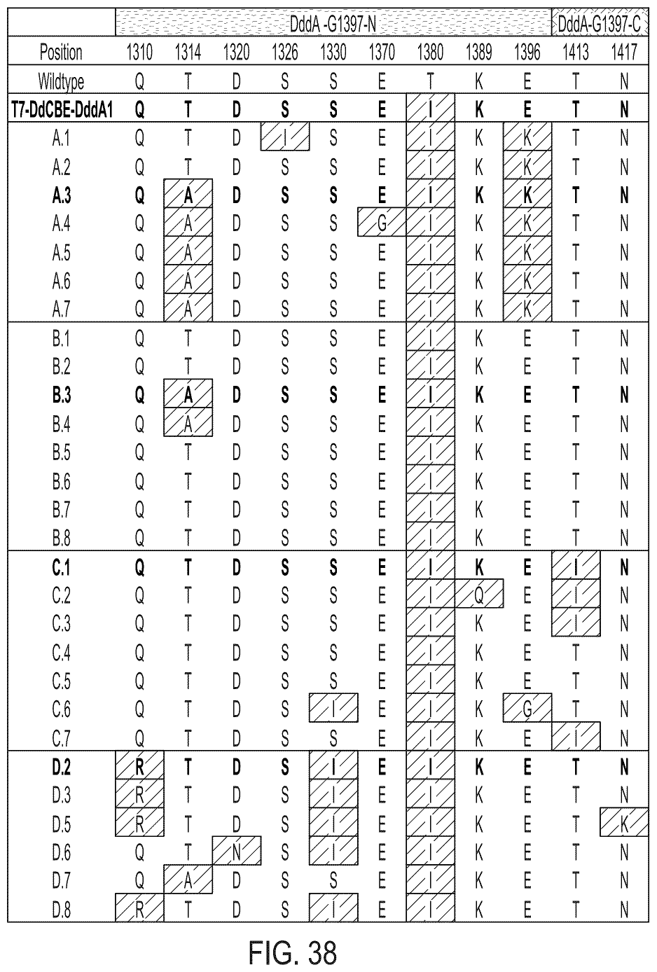

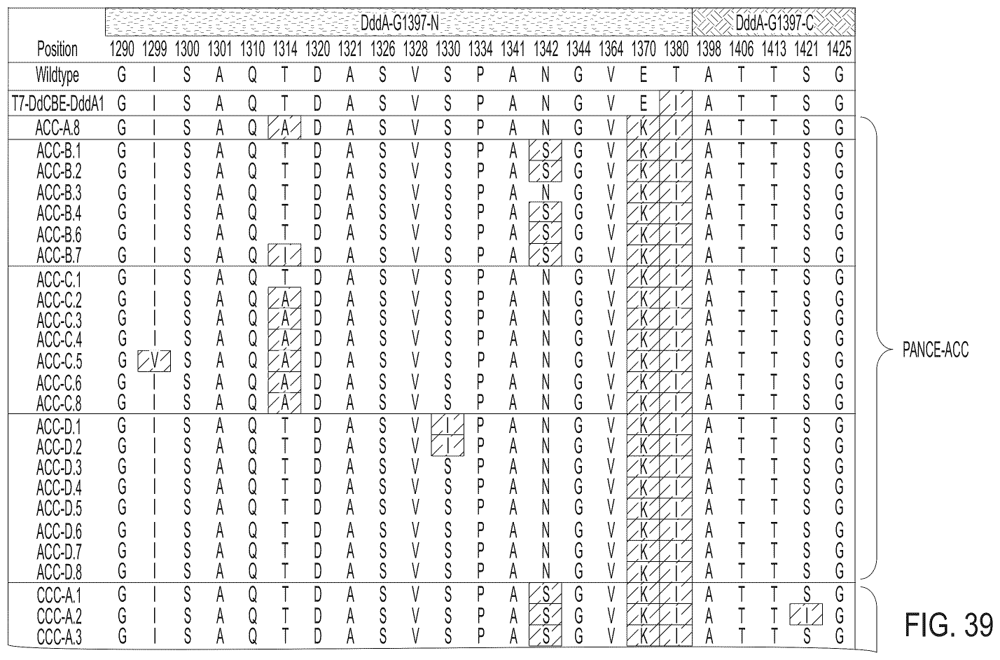

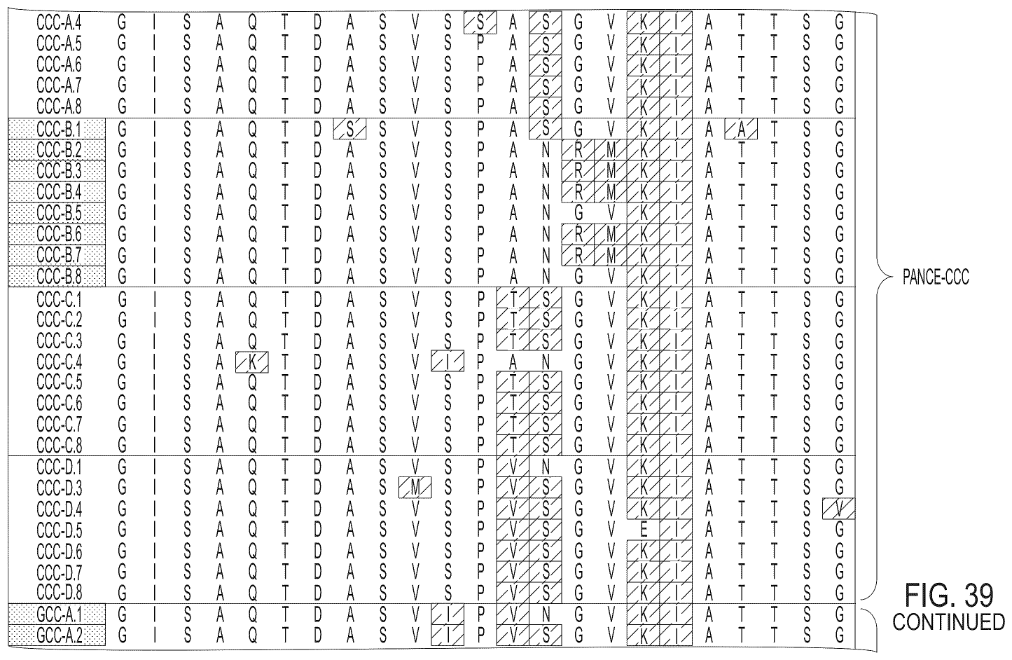

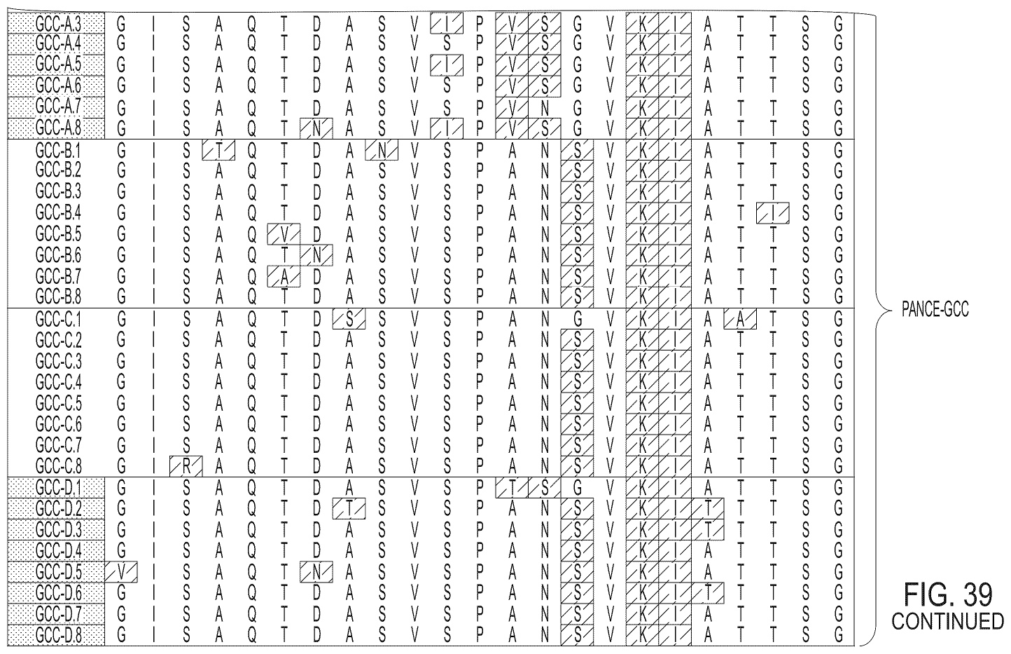

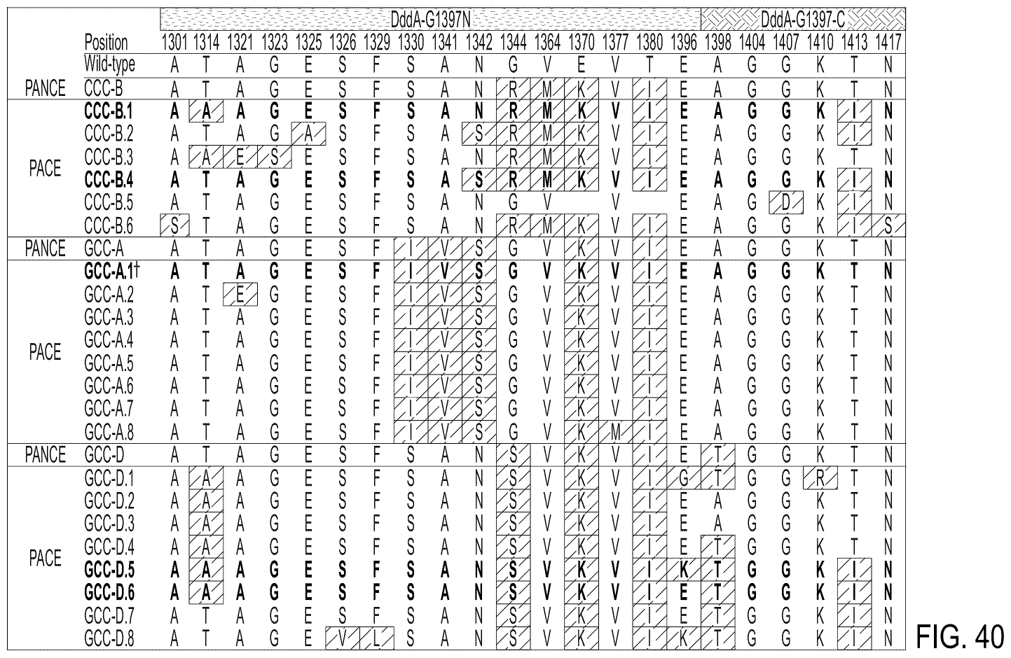

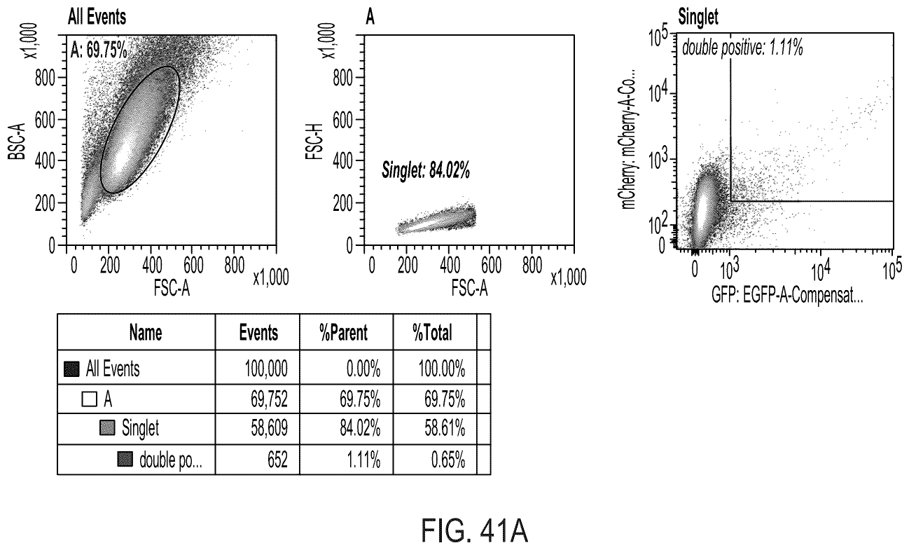

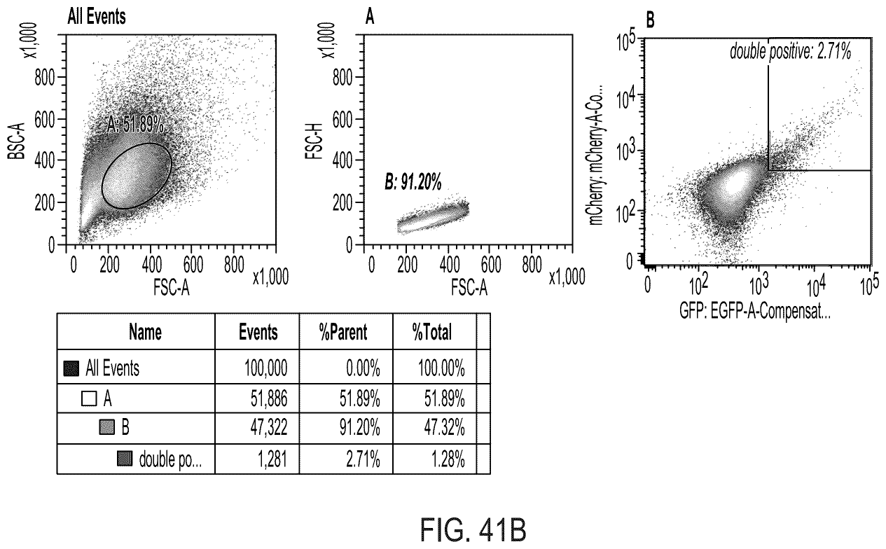

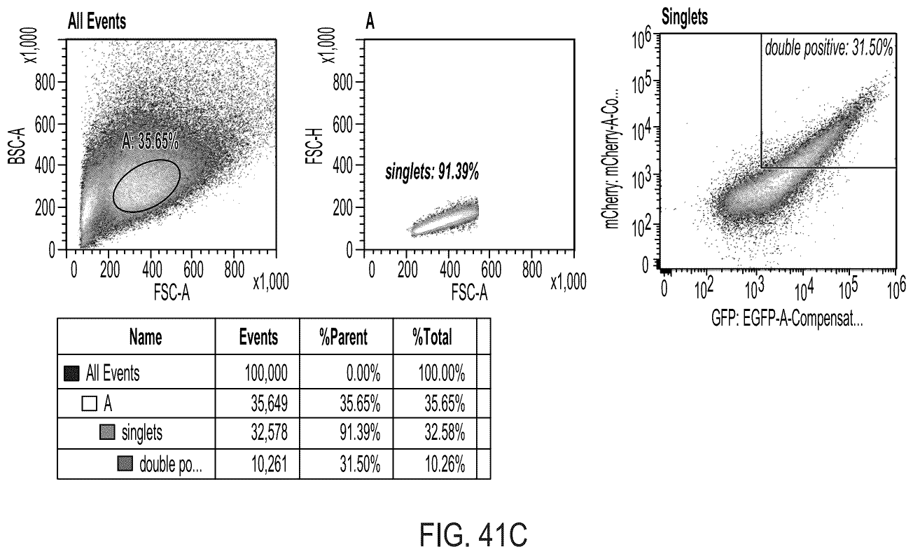

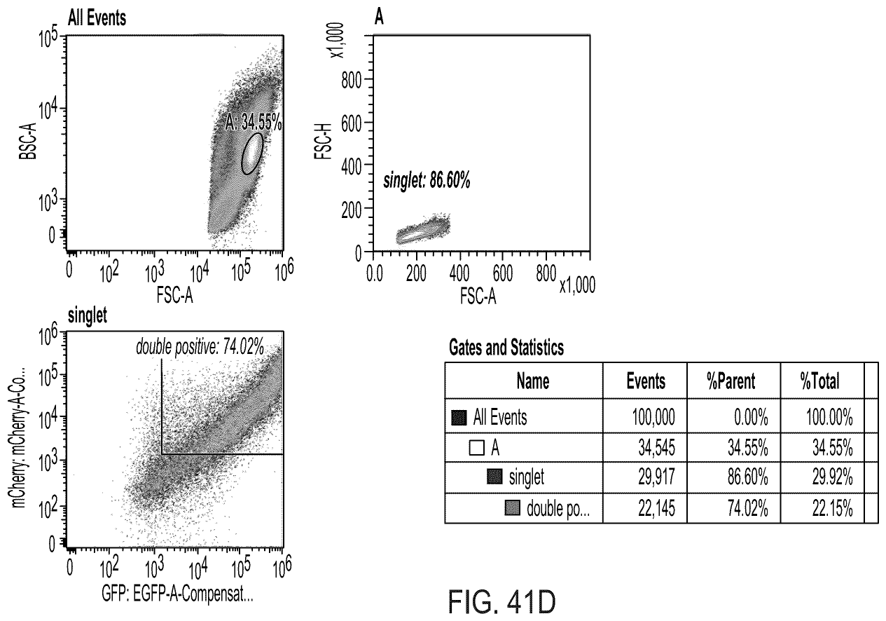

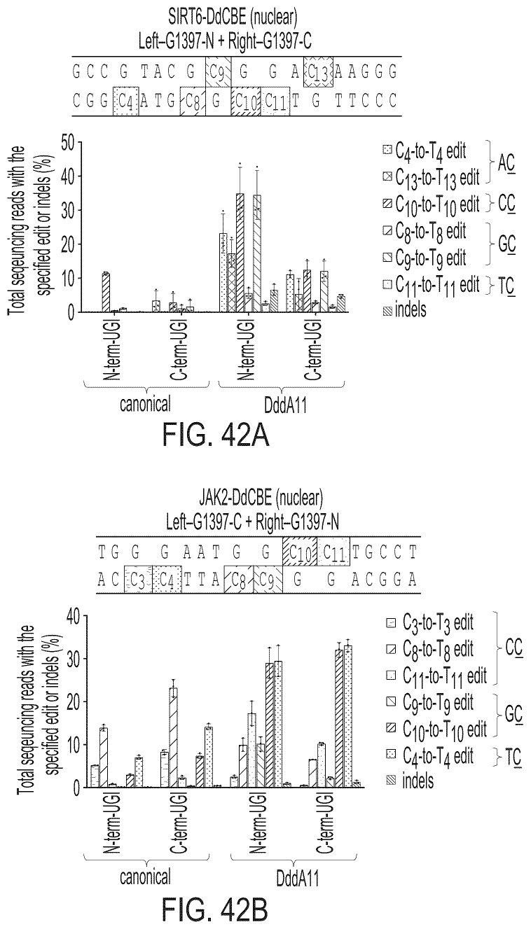

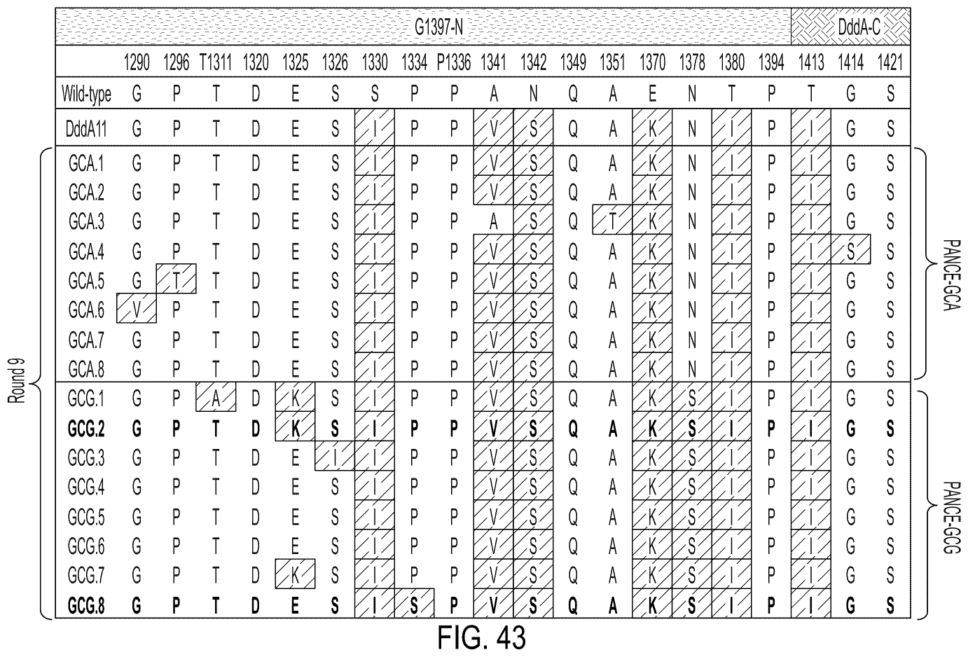

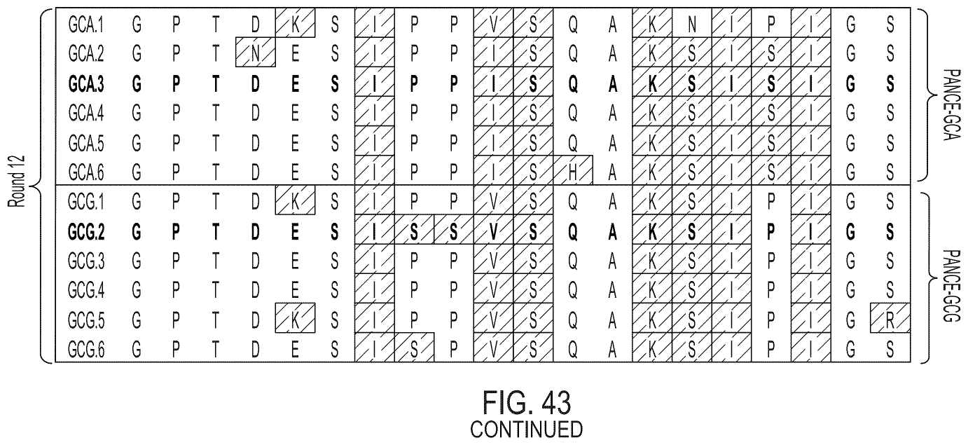

In the context of base editing, all previously described cytidine deaminases utilize single-stranded DNA as a substrate (e.g., the R-loop region of a Cas9-gRNA/dsDNA complex). Base editing in the context of mitochondrial DNA has not heretofore been possible due to the challenges of introducing and/or expressing the gRNA needed for a Cas9-based system into mitochondria. The inventors have recognized for the first time that the catalytic properties of DddA can be leveraged to conduct base editing directly on a double strand DNA substrate by separating the DddA into inactive portions, which when co-localized within a cell will become reconstituted as an active DddA. This avoids or at least minimizes the toxicity associated with delivering and/or expressing a fully active DddA in a cell. For example, a DddA may be divided into two fragments at a “split site,” i.e., a peptide bond between two adjacent residues in the primary structure or sequence of a DddA. The split site may be positioned anywhere along the length of the DddA amino acid sequence, so long as the resulting fragments do not on their own possess a toxic property (which could be a complete or partial deaminase activity). In certain embodiments, the split site is located in a loop region of the DddA protein. In the embodiment shown in FIG. 1A, the arrows depict five possible split sites approximately equally spaced along the length of the DddA protein. The depicted embodiment further shows that the DddA was divided into two fragments at a split site located approximately in the middle of the DddA amino acid sequence. The DddA fragment lying to the left of the split site may be referred to as the “N-terminal DddA half” and the DddA fragment lying to the right of the split site may be referred to as the “C-terminal DddA half.” FIG. 1A identifies these fragments as “DddA half^{A}” and DddA half^{B}”, respectively. Depending on the location of the split site, the N-terminal DddA half and the C-terminal DddA half could be the same size, approximately the same size, or very different sizes.

FIG. 1B depicts a pair of Evolved DddA-containing base editors each comprising a pDNAbp (pDNAbp A and pDNAbp B) fused to an inactive fragment of DddA (DddA half^{A} and DddA half^{B}). The pDNAbp components bind to their cognate target sites (target site A and target site B) on the mtDNA, thereby localizing the inactive DddA fragments at the target edit/deamination site. Once localized, the DddA activity is restored. It should be noted, that while the pDNAbpA is shown binding to a target site which is physically arranged on the same side of the deamination site as the DddA half^{A}, the DddA half^{A} may be physically arranged so that it approaches the deamination site (e.g., for reconstitution) from any side (e.g., same side, top, opposite side, bottom, or any other angle to the deamination site (e.g., off-axis)) such that it may reconstitute with its DddA half^{B}. Additionally, while the figure shows the pDNAbpA and pDNAbpB binding to target sites on opposite sides of the deamination site, it can be readily envisioned that in view of the aforementioned description regarding orientation, that the two pDNAbp (e.g., A and B) may bind on the same side of the deamination site or opposite sides, provided that the DddA halves may reconstitute and effect deamination at the deamination site. Moreover, while the figure shows the pDNAbpA and pDNAbpB binding to target sites on opposite strands of the DNA duplex, it can be readily envisioned that in view of the aforementioned description regarding orientation, that the two pDNAbp (e.g., A and B) may bind on the same strand of the DNA duplex or opposite strands, provided that the DddA halves may reconstitute and effect deamination at the deamination site. Using these premises, it can readily be envisioned that in some embodiments, the DddA halves are oriented in any position relative to the deamination site such that they effectuate deamination, and further that the pDNAbp to which they are linked may be on the same side or different side of the deamination site, and in some embodiments, such pDNAbp of each of the DddA halves are on the same side of the deamination site, on different sides of the deamination site, are on the same strand of the DNA duplex, or on different strands of the DNA duplex.

FIG. 1C depicts a pair of Evolved DddA-containing base editors each comprising a mitoTALE (mitoTALE A and mitoTALE B) fused to an inactive fragment of DddA (DddA half^{A} and DddA half^{B}). The mitoTALE components bind to their cognate target sites (target site A and target site B) on the mtDNA, thereby localizing the inactive DddA fragments at the target edit/deamination site. Once localized, the DddA activity is restored. It should be noted, that while the mitoTALEA is shown binding to a target site which is physically arranged on the same side of the deamination site as the DddA half^{A}, the DddA half^{A} may be physically arranged so that it approaches the deamination site (e.g., for reconstitution) from any side (e.g., same side, top, opposite side, bottom, or any other angle to the deamination site (e.g., off-axis)) such that it may reconstitute with its DddA half^{B}. Additionally, while the figure shows the mitoTALEA and mitoTALEB binding to target sites on opposite sides of the deamination site, it can be readily envisioned that in view of the aforementioned description regarding orientation, that the two mitoTALE (e.g., A and B) may bind on the same side of the deamination site or opposite sides, provided that the DddA halves may reconstitute and effect deamination at the deamination site. Moreover, while the figure shows the mitoTALEA and mitoTALEB binding to target sites on opposite strands of the DNA duplex, it can be readily envisioned that in view of the aforementioned description regarding orientation, that the two mitoTALE (e.g., A and B) may bind on the same strand of the DNA duplex or opposite strands, provided that the DddA halves may reconstitute and effect deamination at the deamination site. Using these premises, it can readily be envisioned that in some embodiments, the DddA halves are oriented in any position relative to the deamination site such that they effectuate deamination, and further that the mitoTALE to which they are linked may be on the same side or different side of the deamination site, and in some embodiments, such mitoTALE of each of the DddA halves are on the same side of the deamination site, on different sides of the deamination site, are on the same strand of the DNA duplex, or are on different strands of the DNA duplex.

FIG. 1D depicts a pair of Evolved DddA-containing base editors each comprising a mitoZFP (mitoZFP A and mitoZFP B) fused to an inactive fragment of DddA (DddA half^{A} and DddA half^{B}). The mitoZFP components bind to their cognate target sites (target site A and target site B) on the mtDNA, thereby localizing the inactive DddA fragments at the target edit/deamination site. Once localized, the DddA activity is restored. It should be noted, that while the ZFPA is shown binding to a target site which is physically arranged on the same side of the deamination site as the DddA half^{A}, the DddA half^{A} may be physically arranged so that it approaches the deamination site (e.g., for reconstitution) from any side (e.g., same side, top, opposite side, bottom, or any other angle to the deamination site (e.g., off-axis)) such that it may reconstitute with its DddA half^{B}. Additionally, while the figure shows the ZFPA and ZFPB binding to target sites on opposite sides of the deamination site, it can be readily envisioned that in view of the aforementioned description regarding orientation, that the two ZFP (e.g., A and B) may bind on the same side of the deamination site or opposite sides, provided that the DddA halves may reconstitute and effect deamination at the deamination site. Moreover, while the figure shows the ZFPA and ZFPB binding to target sites on opposite strands of the DNA duplex, it can be readily envisioned that in view of the aforementioned description regarding orientation, that the two ZFP (e.g., A and B) may bind on the same strand of the DNA duplex or opposite strands, provided that the DddA halves may reconstitute and effect deamination at the deamination site. Using these premises, it can readily be envisioned that in some embodiments, the DddA halves are oriented in any position relative to the deamination site such that they effectuate deamination, and further that the ZFP to which they are linked may be on the same side or different side of the deamination site, and in some embodiments, such ZFP of each of the DddA halves are on the same side of the deamination site, on different sides of the deamination site, are on the same strand of the DNA duplex, or are on different strands of the DNA duplex.

FIG. 1E depicts a pair of Evolved DddA-containing base editors each comprising a Cas9 (Cas9 A and Cas9 B) fused to an inactive fragment of DddA (DddA half^{A} and DddA half^{B}). The Cas9 components bind to their cognate target sites (target site A and target site B) on the mtDNA as programmed by their respective guide RNAs, thereby localizing the inactive DddA fragments at the target edit/deamination site. Once localized, the DddA activity is restored. It should be noted, that while the Cas9A is shown binding to a target site which is physically arranged on the same side of the deamination site as the DddA half^{A}, the DddA half^{A} may be physically arranged so that it approaches the deamination site (e.g., for reconstitution) from any side (e.g., same side, top, opposite side, bottom, or any other angle to the deamination site (e.g., off-axis)) such that it may reconstitute with its DddA half^{B}. Additionally, while the figure shows the Cas9A and Cas9B binding to target sites on opposite sides of the deamination site, it can be readily envisioned that in view of the aforementioned description regarding orientation, that the two Cas9 (e.g., A and B) may bind on the same side of the deamination site or opposite sides, provided that the DddA halves may reconstitute and effect deamination at the deamination site. Moreover, while the figure shows the Cas9A and Cas9B binding to target sites on opposite strands of the DNA duplex, it can be readily envisioned that in view of the aforementioned description regarding orientation, that the two Cas9 (e.g., A and B) may bind on the same strand of the DNA duplex or opposite strands, provided that the DddA halves may reconstitute and effect deamination at the deamination site. Using these premises, it can readily be envisioned that in some embodiments, the DddA halves are oriented in any position relative to the deamination site such that they effectuate deamination, and further that the Cas9 to which they are linked may be on the same side or different side of the deamination site, and in some embodiments, such Cas9 of each of the DddA halves are on the same side of the deamination site, on different sides of the deamination site, are on the same strand of the DNA duplex, or are on different strands of the DNA duplex.

FIGS. 1F-1I. depicts a variety of architectural embodiments envisioned for the constructs described in any of FIGS. 1A to 1E. These architectural embodiments are not intended to limit the present disclosure as other architectures are also feasible and are contemplated by this disclosure. Embodiment (a) depicts a first fusion protein comprising a pDNAbp (arbitrarily labeled pDNAbp A) fused to a DddA half domain (arbitrarily labeled DddA half A) which binds to a first target site on a strand of a double-stranded DNA molecule (e.g., a miDNA). The first target site is arbitrarily labeled “target site A.” This embodiment also depicts a second fusion protein comprising a second pDNAbp (i.e., pDNAbp B) fused through a linker to a second DddA half (i.e., DddA half B). The second fusion protein is shown binding to a second target site on the opposite strand of DNA as the first target site. The DddA half A and DddA half B associate at the deamination site (“*”) to form a functional DddA which then proceeds to deaminate the deamination site. As illustrated by architectural embodiments in (a) through (e), the target sites are located on opposite strands of the DNA, with the pDNAbps binding to opposite strands. Embodiments (f) through (k), however, show that the target sites may be located on the same strand, with the pDNAbps binding to the same strands. In some embodiments, such as in (f) through (i), the target sites to which the pDNAbps bind are located on the same strand containing the target deamination site (“*”). In other embodiments, as depicted in (i) through (k), the target sites to which the pDNAbps bind are located on the strand opposite the strand containing the target deamination site (“*”). In addition, the fusion proteins can be arranged in any suitable linear order of domains, including N-[dDNAbp]-[linker]-[DddA half]-C and N-[DddA half]-[linker]-[dDNAbp]-C. Still further, the fusion proteins may be configured such that the DddA halves (e.g., DddA half A and DddA half B) associate near or adjacent the deamination target site, such as in same-side association near the deamination site in (d) or (f), or opposite-side association opposite the deamination site in (e) and (i), or combinations of these configurations, as in (a), (b), (c), (g), (h), (j), (k), or (l) through (q). In addition, the linker may fuse the Evolved DddA domain to either side of the pDNAbp, as shown in the variations of (l) through (q), or combinations of these embodiments. In addition, the DddA halves may associate with one another on either side of the target deamination site (e.g., compare embodiment (r) versus any of the embodiments of (a) through (q). The disclosure is not limited to the embodiments depicted.

FIG. 2 is a schematic showing the selection circuit in PANCE or PACE for evolving split DddA towards higher activity at TC context. DdCBE is encoded in M13 bacteriophage. Plasmid P3 is in the E. coli host cell and encodes for T7 RNA polymerase (T7 RNAP) fused to a degron. TALE-3 and TALE-4 target DNA sequences flanking a linker region within the T7 RNAP-degron fusion. Successful base editing at the linker sequence introduces a stop to remove the degron from T7 RNAP during translation. T7 RNAP is restored and binds to the T7 promoter on Plasmid P4 to drive gIII. Since gIII is required for phage infectivity, phages containing active DdCBEs will propagate and overtime.

FIGS. 3A-3D show editing activity of DdCBE mutants in mammalian HEK293T cells. FIG. 3A shows DdCBE protein architecture used to test mutant activity. FIGS. 3B-3C show editing efficiencies of DdCBEs targeting MT-ATP8, MT-ND5.2 and MT-ND4 3-days post transfection.

FIG. 3D shows indel percentages associated with DdCBE editing.

FIG. 4 is a chart showing the mutations of DddA variants. RIII and II were evolved on 5′-TCC. CC variants were evolved on 5′-CCC. GC variants were evolved on 5′-GCC.

FIGS. 5A-5B show DddA mutations after PACE. The T1380I mutation was obtained from earlier rounds of optimization and was incorporated into input phage for final PACE. Mutations E1396K and T1413I were obtained along the DddA-split interface.

FIGS. 6A-6B show that selected DddA mutants improve TC editing efficiency.

FIGS. 7A-7C show that RII DddA mutants improve editing at multiple mtDNA sites.

FIGS. 8A-8C show that RII mutants are compatible with G1333 split.

FIG. 9 shows a reversion analysis of DddA mutants for improved activity at TC. RIII and III variants, that showed consistent improvement in DddA activity at TC across multiple sites, are boxed.

FIGS. 10A-10B show that PACE selection circuit expands DddA targeting scope.

FIGS. 11A-11B show PACE mutations of DddA variants evolved against CCC and GCC.

FIG. 12 shows DdCBE library to profile context preference.

FIG. 13 shows that CC mutants are active at HCN contexts.

FIG. 14 shows that GC mutants are active at HCN and inactive at GCN contexts.

FIG. 15 shows a summary of bacteria profiling assay. Results show that CC-3 is the most active mutant at 5′-CC.

FIG. 16 shows mtDNA-ATP8 editing in HEK293T. Results show that GC-3 is the most active mutant at HCN contexts.

FIG. 17 shows mtDNA-ND5.2 editing in HEK293T. Results show that GC-3 is the most active mutant at HCN contexts.

FIG. 18 shows the suggested mutants for biochemical characterization. RIII and III show higher TC activity. GC-3 and CC-2 show higher CC activity.

FIGS. 19A-19C show phage-assisted evolution of DddA-derived cytosine base editor for improved activity and expanded targeting scope. FIG. 19A shows the selection to evolve DdCBE using PANCE and PACE. An accessory plasmid (AP, purple) contains gene III driven by the T7 promoter. The complementary plasmid (CP, orange) expresses a T7 RNAP-degron fusion. The evolving T7-DdCBE containing DddA split at G1397 is encoded in the selection phage (SP, blue). MP6, mutagenesis plasmid. Where relevant, the promoters are indicated. FIG. 19B shows a 2-amino-acid linker connects T7 RNAP to the degron. The linker sequence contains cytidines C₆ and C₇ that are targets for DdCBE editing. The nucleotide at position 8 can be varied to T, A, C or G to form plasmids CP-TCC, CP-ACC, CP-CCC and CP-GCC, respectively. In the absence of target C-to-T editing, expression of degron (brown) results in proteolysis of T7 RNAP (orange) and inhibition of gIII expression. Active T7-DdCBE edits one or both target cytidines to install a stop codon (*) within the linker, thus restoring active T7 RNAP to mediate gIII expression. FIG. 19C shows the architecture of T7-DdCBE and the 15-bp target spacing region. Nucleotides corresponding to DNA sequences within T7 RNAP, linker and degron genes are colored in orange, gray and brown, respectively.

FIGS. 20A-20F show evolved DddA variants improve mitochondrial base editing activity at 5′-TC. FIG. 20A shows mutations within the DddA gene of T7-DdCBE. Variants were isolated after evolution of canonical T7-DdCBE using PANCE and PACE in strain 4 transformed with MP6 (see FIG. 23A). DddA6 was rationally designed by incorporating the T1413I mutation into DddA5. FIG. 20B shows the crystal structure of DddA (grey, PDB 6U08) complexed with DddI immunity protein (not shown). Positions of mutations enriched after PANCE and PACE are colored in orange. The catalytic residue E1347 is shown. DddA was split at G1397 (red) to generate T7-DdCBE. FIGS. 20C-20D show mitochondrial DNA editing efficiencies and indel frequencies of HEK293T cells treated with (FIG. 20C) ND5.2-DdCBE or (FIG. 20D) ATP8-DdCBE. The genotypes of DddA variants correspond to FIG. 20A. For each base editor, the DNA spacing region, target cytosines and DddA split orientation are shown. FIG. 20E shows Frequencies of MT-ND5 alleles produced by DddA6 in FIG. 20C. FIG. 20F shows Frequencies of MT-ATP8 alleles produced by DddA6 in FIG. 20D. For FIGS. 20C-20F, values and errors reflect the mean±s.d. of n=3 independent biological replicates.

FIGS. 21A-21F show evolved DddA variants show enhanced editing at TC and non-TC target sequences in mitochondrial and nuclear DNA. FIG. 21A shows a bacterial assay to profile sequence preferences of evolved DddA variants. E. coli host cells expressing both halves of canonical or evolved T7-DdCBE were electroporated with a 16-membered library of NC₇N target plasmids for base editing. Target plasmids were isolated after overnight incubation for high-throughput sequencing of the spacing region (pink highlight). FIG. 21B shows a heat map showing C⋅G-to-T⋅A editing efficiencies of NC₇N sequence in each target plasmid. Target cytosines in all 16 possible NC₇N sequences, including the second cytosine in NCC₆ sequences, are colored in purple. Genotypes of listed variants correspond to FIG. 20A and FIG. 21C. Mock-treated cells did not express T7-DdCBE and contained only the library of target plasmids. Shading levels reflect the mean of n=3 independent biological replicates. FIG. 21C shows genotypes of DddA variants after evolving T7-DdCBE-DddA1 using context-specific PANCE and PACE. Mutations enriched for activity on a CCC linker or GCC linker are highlighted in red and blue, respectively. FIGS. 21D-21E show mitochondrial C⋅G-to-T⋅A editing efficiencies of HEK293T cells treated with canonical and evolved variants of (FIG. 21D) ND5.2-DdCBE or (FIG. 21E) ATP8-DdCBE. Target spacing regions and split DddA orientations are shown for each base editor. Cytosines highlighted in light purple and dark purple are in non-TC contexts. FIG. 21F shows the approximate editing windows for canonical (purple), DddA6 (red) and DddA11 (blue) variants of T7-DdCBE containing the G1397 split. The length of each colored line reflects the approximate relative editing efficiency for each DddA variant. FIGS. 21G-21H show nuclear DNA editing efficiencies of HEK293T cells treated with the canonical or DddA11 variant of (FIG. 21G) SIRT6-DdCBE or (FIG. 21H) JAK2-DdCBE. Target spacing regions and split DddA orientations are shown for each base editor. Cytosines highlighted in yellow, red, or blue are in AC, CC̅, or GC̅ contexts, respectively. The architecture of each nuclear DdCBE half is bpNLS-2×UGI-4-amino-acid linker-TALE-[DddA half]. bpNLS, bipartite nuclear localization signal. FIG. 21I shows the average percentage of genome-wide C⋅G-to-T⋅A off-target editing in mtDNA for indicated DdCBE and controls in HEK293T cells. For FIGS. 21D-21E and FIGS. 21G-21I, values and error bars reflect the mean±s.d of n=3 independent biological replicates.

FIGS. 22A-22F show the application of DddA11 variant to install pathogenic mutations at non-TC targets in HEK293T cells. FIG. 22A shows the use of DdCBEs to install disease-associated target mutations in human mtDNA. (V, valine; I, isoleucine; A, alanine; T, threonine; Q, glutamine; *, stop). FIGS. 22B-22D show mitochondrial base editing efficiencies of HEK293T cells treated with canonical or evolved (FIG. 22B) ND4.3-DdCBE, (FIG. 22C) ND4.2-DdCBE and (FIG. 22D) ND5.4-DdCBE. On-target cytosines are colored green, blue, or red, respectively. Cells expressing the DddA11 variant of DdCBE were isolated by fluorescence-activated cell sorting for high-throughput sequencing. The split orientation, target spacing region, and corresponding encoded amino acids are shown. Nucleotide sequences boxed in dotted lines are part of the TALE-binding site. FIGS. 22E-22F show oxygen consumption rate (OCR) (FIG. 22E) and relative values of respiratory parameters (FIG. 22F) in sorted HEK293T cells treated with the DddA11 variant of ND4.2-DdCBE or ND5.4-DdCBE. For FIGS. 22B-22F, values and error bars reflect the mean±s.d of n=3 independent biological replicates, except ND4.2-DdCBE in FIG. 22E and FIG. 22F reflect n=2 independent biological replicates. *P<0.05; **P<0.01, ***P<0.001 by Student's unpaired two-tailed t-test.

FIGS. 23A-23D show the evolution of canonical T7-DdCBE for improved TC activity using PANCE. FIG. 23A shows strains for screening selection stringency. Strains were generated by transformation with a variant of an AP and a variant of a CP. All CPs encode a TCC linker. Relative RBS strengths of SD8, sd8, sd2 and sd4U are 1.0, 0.20, 0.010 and 0.00040, respectively. FIG. 23B shows overnight phage propagation of indicated SPs in host strains with increasing stringencies. Dead T7-DdCBE phage contained the catalytically inactivating E1347A mutation in DddA. The fold phage propagation is the output phage titer divided by the input titer. FIG. 23C shows phage passage schedule for canonical T7-DdCBE evolution in PANCE using strain 4 transformed with MP6. Table indicates the dilution factor for the input phage population. Output phage titers for each replicate (A, B, C and D) are shown for each passage. Average fold propagation was obtained by averaging the fold propagation obtained from the four replicates A-D. FIG. 23D shows mitochondrial base editing efficiencies of HEK293T cells treated with canonical DdCBE or with DdCBEs containing the indicated mutations within DddA. For each base editor, the DddA split orientation and target cytosine (purple) within the spacing region is indicated. For FIG. 23B and FIG. 23D values and error bars reflect the mean±s.d of n=3 independent biological replicates.

FIGS. 24A-24D show DddA6 is compatible with split-G1333 and split-G1397 DdCBE orientations. Mitochondrial base editing efficiencies of HEK293T cells treated with (FIG. 24A) ND1.1-DdCBE, (FIG. 24B) ND1.2-DdCBE, (FIG. 24C) ND2-DdCBE and (FIG. 24D) ND4-DdCBE. Target spacing regions and split DddA orientations are shown above each plot. Values and error bars reflect the mean±s.d of n=3 independent biological replicates.

FIGS. 25A-25E show the evolution of DddA1-containing T7-DdCBE for expanded targeting scope using PANCE. FIG. 25A shows strains for overnight phage propagation assays on non-TC linker substrates. FIG. 25B shows overnight fold propagation of indicated SP in host strains encoding TC or non-TC linkers. Strains correspond to FIG. 25A. T7-DdCBE-DddA1 phage harbors a T1380I mutation in DddA. Dead T7-DdCBE-DddA1 phage contains an additional catalytically inactivating E1347A mutation in DddA. Values and error bars reflect the mean±s.d of n=3 independent biological replicates. FIGS. 25C-25E show phage passage schedule for T7-DdCBE-DddA1 evolution in PANCE using (FIG. 25C) strain 5 transformed with MP6, (FIG. 25D) strain 6 transformed with MP6 or (FIG. 25E) strain 7 transformed with MP6. Tables indicate the dilution factor for the input phage population. To initiate drift, phage from the previous passage was diluted 2 to 5-fold by mixing with log-phase cells containing pJC175e-DddI (see Example 2, Methods) and MP6. Phage was isolated after drifting for ˜8 h and mixed with the respective selection host strain for activity-dependent overnight phage propagation. For a given linker target, the output phage titers for each replicate (A, B, C and D) are shown for each passage. Average fold propagations above the dotted line in each graph represent propagation >1-fold. Average fold propagation was obtained by averaging the fold propagation obtained from the four replicates.

FIGS. 26A-26D show allele compositions from mitochondrial and nuclear editing by DddA11-containing DdCBEs. FIG. 26A shows frequencies of mitochondrial MT-ND5 alleles produced by DddA11 variant of ND5.2-DdCBE. FIG. 26B shows frequencies of mitochondrial MT-ATP8 alleles produced by DddA11 variant of ATP8-DdCBE. FIG. 26C shows frequencies of nuclear SIRT6 alleles produced by DddA11 variant of SIRT6-DdCBE. FIG. 26D shows frequencies of nuclear JAK2 alleles produced by DddA11 variant of JAK2-DdCBE. Values and errors reflect the mean±s.d of n=3 independent biological replicates.

FIGS. 27A-27C show evolved DddA variants mediate mitochondrial base editing in multiple human cell lines. Mitochondrial DNA editing efficiencies of canonical and evolved ND5.2-DdCBE in (FIG. 27A) HeLa cells, (FIG. 27B) K562 cells, and (FIG. 27C) U2OS cells. Editing efficiencies were measured for unsorted cells (bulk) and isolated DdCBE-expressing cells (sorted). Target spacing region and split DddA orientation are shown. Cytosines highlighted in light purple and dark purple are in non-TC contexts. Values and error bars reflect the mean±s.d of n=3 independent biological replicates.

FIG. 28 shows reversion analysis of DddA11. Mitochondrial base editing efficiencies of reversion mutants from ATP8-DdCBE-DddA11 (labelled as 11) in HEK293T cells. Reversion mutants are designated 11a-11h. Amino acids that differ from those in canonical ATP8-DdCBE are indicated, so the absence of an amino acid indicates a reversion to the corresponding canonical amino acid in the first column. Values and error bars reflect the mean±s.d of n=3 independent biological replicates.

FIGS. 29A-29H show editing windows of canonical and evolved T7-DdCBE. FIG. 29A shows target spacing region recognized by T7-DdCBE. Each spacing region contains TC repeats within the top strand (left, solid line) or bottom strand (right, dashed line). Lengths of spacing regions ranged from 12-18-bp. FIGS. 29B-29H show editing efficiencies mediated by canonical DdCBE (purple), DddA6-containing DdCBE (red) and DddA11-containing DdCBE (blue) are shown for each cytosine positioned within the spacing region length of (FIG. 29B) 12-bp, (FIG. 29C) 13-bp, (FIG. 29D) 14-bp, (FIG. 29E) 15-bp, (FIG. 29F) 16-bp, (FIG. 29G) 17-bp and (FIG. 29H) 18-bp. Subscripted numbers refer to the positions of cytosines in the spacing region, counting the DNA nucleotide immediately after the binding site of TALE3 as position 1. Editing efficiencies associated with the top and bottom strand are shown as solid and dashed lines, respectively. Mock-treated cells contained only the library of target plasmids (grey). For FIGS. 29B-29H, values and error bars reflect the mean±s.d of n=3 independent biological replicates

FIGS. 30A-30B show editing efficiencies at predicted nuclear off-target sites for nuclear-targeting DdCBEs. Average frequencies of all possible C⋅G-to-T⋅A conversions within a predicted off-target spacing region associated with (FIG. 30A) SIRT6-DdCBE and (FIG. 30B) JAK2-DdCBE. Values and error bars reflect the mean±s.d of n=3 independent biological replicates. See Table 9 for ranking of predicted off-target sites and Table 7 for off-target site amplicons.

FIGS. 31A-31D show the evolution of T7-DdCBE-DddA11 using PANCE for improved GC activity. FIG. 31A shows the sequence encoding the T7 RNAP-degron linker was modified to contain GCA or GCG in an effort to evolve for higher activity on GC targets. T7-DdCBE must convert GC₈ to GT₈ to install a stop codon in the linker sequence and restore T7 RNAP activity. FIG. 31B shows strains for overnight phage propagation assays on GCA or GCG linkers. FIG. 31C shows overnight fold propagation of indicated SP in host strains encoding GCA or GCG linkers. Strains correspond to FIG. 31B. T7-DdCBE-DddA11 phage contains the mutations S1330I, A1341V, N1342S, E1370K, T1380I and T1413I in DddA. Dead T7-DdCBE-DddA11 phage contains an additional inactivating E1347A mutation in DddA. Values and error bars reflect the mean±s.d of n=3 independent biological replicates. FIG. 31D shows the phage passage schedule for T7-DdCBE-DddA11 evolution in PANCE using strain 9 transformed with MP6 (red) or strain 10 transformed with MP6 (blue). The table indicates the dilution factor for the input phage population. To initiate drift, phage from the previous passage was diluted 2-fold by mixing with log-phase cells containing pJC175e-DddI (see Example 2, Methods) and MP6. Phage were isolated after drifting for ˜8 h and mixed with the respective selection host strain for activity-dependent overnight phage propagation. Output phage titer and fold propagation are shown for a single replicate. Fold propagations above the dotted line in each graph represent propagation >1-fold.

FIGS. 32A-32E show mitochondrial editing efficiencies of DdCBE variants evolved from GC-specific PANCE. FIG. 32A shows enriched mutations within the DddA gene of T7-DdCBE after PANCE against a GCA or GCG linker. T7-DdCBE-DddA11 was used as the input SP for PANCE. DddA mutations in the input SP are shown in beige. Mutations enriched after 9 or 12 PANCE passages are shown in blue. FIGS. 32B-32E show heat maps of mitochondrial base editing efficiencies of HEK293T cells treated with canonical and evolved variants of (FIG. 32B) ND4.3-DdCBE, (FIG. 32C) ND5.4-DdCBE (FIG. 32D) ND5.2-DdCBE and (FIG. 32E) ATP8-DdCBE. Target spacing regions and split DddA orientations are shown for each base editor. For FIGS. 32B-32E, colors reflect the mean of n=3 independent biological replicates.

FIGS. 33A-33G show mitochondrial genome-wide off-target C⋅G-to-T⋅A mutations. FIGS. 33A-33F show the average frequency and mitochondrial genome position of each unique C⋅G-to-T⋅A single nucleotide variant (SNV) is shown for HEK293T cells treated with (FIG. 33A) canonical ATP8-DdCBE, (FIG. 33B) ATP8-DdCBE containing DddA6, (FIG. 33C) ATP8-DdCBE containing DddA11, (FIG. 33D) canonical ND5.2-DdCBE, (FIG. 33E) ND5.2-DdCBE containing DddA6 and (FIG. 33F) ND5.2-DdCBE containing DddA11. FIG. 33G shows the ratio of average on-target:off-target editing for the indicated canonical and evolved DdCBE. The ratio was calculated for each treatment condition as: (average frequency of all on-target C⋅G base pairs)+(average frequency of non-target C⋅G base pairs present in the mitochondrial genome).

FIGS. 34A-34C show allele compositions at disease-relevant mtDNA sites in HEK293T cells following base editing by DddA11-containing DdCBE variants. Allele frequency table of HEK293T cells treated with DddA11-containing (FIG. 34A) ND4.3-DdCBE, (FIG. 34B) ND4.2-DdCBE and (FIG. 34C) ND5.4-DdCBE to install the non-TC mutations m.11642G>A, m.11696>A and m.13297G>A, respectively. On-target cytosines are boxed. Values and errors reflect the mean±s.d of n=3 independent biological replicates.

FIGS. 35A-35B show the structural alignment of DddA with ssDNA-bound APOBEC3G. FIG. 35A shows the crystal structure of DddA (grey, PDB 6U08) complexed with DddI immunity protein (not shown). Positions of mutations common to the CCC- and GCC-specific evolutions are colored in purple. Additional mutations are colored according to FIG. 21C. DddA was split at G1397 (red) to generate T7-DdCBE for PANCE and PACE. FIG. 35B shows DddA (PDB 6UO8, grey) was aligned to the catalytic domain of APOBEC3G (PDB 2KBO, red) complexed to its ssDNA 5′-CCA substrate (orange) using Pymol. The target C undergoing deamination by APOBEC3G is indicated as C₀. Reversion analysis on the DddA11 mutant indicated that A1341V, N1342S and E1370K are critical for expanding the targeting scope of DddA (see FIG. 28). D317 (red) confers 5′-CC specificity in APOBEC3G and loop 3 controls the catalytic activity of the APOBEC3G.

FIG. 36 shows a mutation table of variants from PANCE of canonical T7-DdCBE for improved TC activity. Strain 4 transformed with MP6 was infected with input SP encoding the canonical T7-DdCBE (see FIG. 23A). Four plaques from each replicate (A, B, C and D) were sequenced after 7 passages. Mutations are highlighted in blue. Genotypes in red were tested for mitochondrial base editing in human cells (see FIG. 23D).

FIGS. 37A-37B show mitochondrial editing efficiencies of DdCBEs containing a mismatched or non-mismatched terminal TALE repeat. The original right TALE in ND5.2-DdCBE contained an RVD in the terminal repeat that recognized a mismatched thymine instead of guanine, and the original left TALE in ATP8-DdCBE contained a mismatched RVD in the terminal repeat the recognized a mismatched thymine instead of cytosine⁵. To clarify the effect of a mismatched RVD on mitochondrial editing efficiencies, we compared the base editing activities between the DdCBE containing the mismatched RVD (labelled as T) and the variant containing the non-mismatched RVD, which is labelled as G for ND5.2-DdCBE (FIG. 37A) and C for ATP8-DdCBE (FIG. 37B). The editing efficiencies for mismatched DdCBEs containing DddA variants were generally comparable to or resulted in 2-10% higher average editing than the equivalent non-mismatched DdCBE (see FIG. 37A and FIG. 37B). Given that DdCBEs containing DddA6 or DddA11 resulted in similar editing efficiencies when tested as a mismatched TALE or non-mismatched TALE, all preceding figures, except for FIGS. 20C-20D and FIGS. 21D-21E, are produced from using DdCBEs containing the original mismatched RVD. Values and error bars reflect the mean±s.d of n=3 independent biological replicates

FIG. 38 shows a mutation table of variants from PACE of T7-DdCBE-DddA1 for improved TC activity. Strain 4 transformed with MP6 was infected with SP encoding T7-DdCBE-DddA1 (see FIG. 23A). Individual plaques were isolated at the end of PACE and sequenced for their DddA genes. Genotypes in red were tested for mitochondrial base editing in human cells.

FIG. 39 shows a mutation table of variants from PANCE of T7-DdCBE-DddA1 for expanded targeting scope. Strains 5, 6 or 7, which were each transformed with MP6, were used for PANCE-ACC, PANCE-CCC or PANCE-GCC, respectively (see FIG. 25A for strain identities). Each host strain was infected with input SP encoding T7-DdCBE-DddA1. Plaques from each replicate (A, B, C and D) were sequenced after 9 passages. Mutations are highlighted in blue. Phage lagoons highlighted in red were used as inputs for PACE.

FIG. 40 shows a mutation table of variants from the PACE evolution to expand targeting scope. Host strain 6 transformed with MP6 was infected with the phage population CCC-B from PANCE. Host strain 7 transformed with MP6 was infected either phage population GCC-A or GCC-D from, both of which were derived from PANCE (see FIG. 25A for strain identities). The consensus genotypes of input phage populations from PANCE are shown. Data was obtained by sequencing individual plaques isolated at the end of PACE. Genotypes in red were tested for base editing in mammalian cells. ^{†}T1413I was included in this genotype.

FIGS. 41A-41D show representative FACS gating plots for eGFP⁺/mCherry⁺ cells. FACs gating plots for eGFP⁺ and mCherry⁺ cell sorting to isolate HeLa cells (FIG. 41A), K562 cells (FIG. 41B), U20S cells (FIG. 41C) and HEK293T cells (FIG. 41D) expressing both halves of ND5.2-DdCBE. The image data was generated on a Sony LE-MA900 cytometer using Cell Sorter Software v. 3.0.5. Cells were initially gated on population using FSC-A/BSC-A (Gate A), then sorted for singlets using FSC-A/FSC-H (Singlet). Live cells were sorted for by gating mCherry-positive and eGFP-positive cells (Double positive). Single-color eGFP and mCherry controls were used for compensation.

FIGS. 42A-42B show nuclear editing efficiencies of DdCBEs containing N-terminal UGI fusions or C-terminal UGI fusions. It was previously reported that the N-terminal UGI fusion of a nuclear-targeting DdCBE resulted in more efficient nuclear base editing compared to a C-terminal UGI fusion⁶. The editing efficiencies of these two architectures were compared in SIRT6-DdCBE (FIG. 42A) and JAK2-DdCBE (FIG. 42B). Consistent with earlier observations, N-terminal UGI fusions generally yielded higher TC and non-TC editing efficiencies compared to C-terminal fusions, except for canonical JAK2-DdCBE. Values and error bars reflect the mean±s.d of n=3 independent biological replicates

FIG. 43 shows a mutation table of variants from the GC-specific PANCE. Strain 9 transformed with MP6 was used for PANCE-GCA. Strain 10 transformed with MP6 was used for PANCE-GCG (see FIG. 31B for strain identities). Each host strain was infected with input SP encoding the DddA11 variant of T7-DdCBE. Plaques were sequenced after nine and 12 passages. Mutations are highlighted in grey.

### DEFINITIONS

As used herein and in the claims, the singular forms “a,” “an,” and “the” include the singular and the plural reference unless the context clearly indicates otherwise. Thus, for example, a reference to “an agent” includes a single agent and a plurality of such agents.

### Base Editing

“Base editing” refers to genome editing technology that involves the conversion of a specific nucleic acid base into another at a targeted genomic locus (e.g., including in a mtDNA). In certain embodiments, this can be achieved without requiring double-stranded DNA breaks (DSB), or single stranded breaks (i.e., nicking). To date, other genome editing techniques, including CRISPR-based systems, begin with the introduction of a DSB at a locus of interest. Subsequently, cellular DNA repair enzymes mend the break, commonly resulting in random insertions or deletions (indels) of bases at the site of the DSB. However, when the introduction or correction of a point mutation at a target locus is desired rather than stochastic disruption of the entire gene, these genome editing techniques are unsuitable, as correction rates are low (e.g. typically 0.1% to 5%), with the major genome editing products being indels. In order to increase the efficiency of gene correction without simultaneously introducing random indels, the present inventors previously modified the CRISPR/Cas9 system to directly convert one DNA base into another without DSB formation. See, Komor, A. C., et al., Programmable editing of a target base in genomic DNA without double-stranded DNA cleavage. Nature 533, 420-424 (2016), the entire contents of which is incorporated by reference herein.

### Base Editor

The term “base editor (BE)” as used herein, refers to an agent comprising a polypeptide that is capable of making a modification to a base (e.g., A, T, C, G, or U) within a nucleic acid sequence (e.g., mtDNA) that converts one base to another (e.g., A to G, A to C, A to T, C to T, C to G, C to A, G to A, G to C, G to T, T to A, T to C, T to G). In some embodiments, the BE refers to those fusion proteins described herein which are capable of modifying bases directly in mtDNA. Such BEs can also be referred to herein as “evolved-DddA containing base editors” or “mtDNA BEs.”0 Such BEs can refer to those fusion proteins comprising a programmable DNA binding protein (“pDNAbp”) (e.g., a mitoTALE, mitoZFP, or a CRISPR/Cas9) and a double-stranded DNA deaminase (“DddA”) to precisely install nucleotide changes and/or correct pathogenic mutations in mtDNA, rather than destroying the mtDNA with double-strand breaks (DSBs). It should be noted that in some places DddA is referred to as DddE (e.g., FIG. 6 of the accompanying drawings). In these instances, DddE shall be interpreted to refer to DddA as a synonym.

In some embodiments, the base editors contemplated herein comprise a nuclease-inactive Cas9 (dCas9) fused to a deaminase which binds a nucleic acid in a guide RNA-programmed manner via the formation of an R-loop, but does not cleave the nucleic acid. For example, the dCas9 domain of the fusion protein may include a D10A and a H840A mutation (which renders Cas9 capable of cleaving only one strand of a nucleic acid duplex), as described in PCT/US2016/058344, which published as WO 2017/070632 on Apr. 27, 2017 and is incorporated herein by reference in its entirety. The DNA cleavage domain of S. pyogenes Cas9 includes two subdomains, the HNH nuclease subdomain and the RuvC1 subdomain. The HNH subdomain cleaves the strand complementary to the gRNA (the “targeted strand”, or the strand in which editing or deamination occurs), whereas the RuvC1 subdomain cleaves the non-complementary strand containing the PAM sequence (the “non-edited strand”). The RuvC1 mutant D10A generates a nick in the targeted strand, while the HNH mutant H840A generates a nick on the non-edited strand (see Jinek et al., Science, 337:816-821(2012); Qi et al., Cell. 28; 152(5):1173-83 (2013)).

BEs that convert a C to T, in some embodiments, comprise a cytidine deaminase (e.g., a double-stranded DNA deaminase or DddA). A “cytidine deaminase” (including those DddAs disclosed herein) refers to an enzyme that catalyzes the chemical reaction “cytosine+H₂O→uracil+NH₃” or “5-methyl-cytosine+H₂O→thymine+NH₃.” As it may be apparent from the reaction formula, such chemical reactions result in a C to U/T nucleobase change. In the context of a gene, such a nucleotide change, or mutation, may in turn lead to an amino acid change in the protein, which may affect the protein's function, e.g., loss-of-function or gain-of-function. In some embodiments, the C to T nucleobase editor comprises a dCas9 or nCas9 fused to a cytidine deaminase. In some embodiments, the cytidine deaminase domain is fused to the N-terminus of the dCas9 or nCas9.

In some embodiments, the nucleobase editor further comprises a domain that inhibits uracil glycosylase, and/or a nuclear localization signal.

Cas9 domains used in base editing have been described in the following references, the contents of which may be applied in the instant disclosure to modify and/or include in BEs described herein, which can target mtDNA, e.g., in Rees & Liu, Nat Rev Genet. 2018; 19(12):770-788 and Koblan et al., Nat Biotechnol. 2018; 36(9):843-846; as well as U.S. Patent Publication No. 2018/0073012, published Mar. 15, 2018, which issued as U.S. Pat. No. 10,113,163; on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; U.S. Pat. No. 10,077,453, issued Sep. 18, 2018; International Publication No. WO 2019/023680, published Jan. 31, 2019; International Publication No. WO 2018/0176009, published Sep. 27, 2018, International Application No PCT/US2019/033848, filed May 23, 2019, International Application No. PCT/US2019/47996, filed Aug. 23, 2019; International Application No. PCT/US2019/049793, filed Sep. 5, 2019; U.S. Provisional Application No. 62/835,490, filed Apr. 17, 2019; International Application No. PCT/US2019/61685, filed Nov. 15, 2019; International Application No. PCT/US2019/57956, filed Oct. 24, 2019; U.S. Provisional Application No. 62/858,958, filed Jun. 7, 2019; International Publication No. PCT/US2019/58678, filed Oct. 29, 2019, the contents of each of which are incorporated herein by reference in their entireties.

Exemplary adenine and cytosine base editors are also described in Rees & Liu, Base editing: precision chemistry on the genome and transcriptome of living cells, Nat. Rev. Genet. 2018; 19(12):770-788; as well as U.S. Patent Publication No. 2018/0073012, published Mar. 15, 2018, which issued as U.S. Pat. No. 10,113,163, on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; and U.S. Pat. No. 10,077,453, issued Sep. 18, 2018, PCT Application PCT/US2017/045381, filed Aug. 3, 2017, which published as WO 2018/027078, and PCT Application No. PCT/US2019/033848, which published as WO 2019/226953, each of which is herein incorporated by reference. Any of the deaminase components of these adenine or cytidine BEs could be modified using a method of directed evolution (e.g., PACE or PANCE) to obtain a deaminase which may use double-stranded DNA as a substrate, and thus, which could be used in the BEs described herein which are intended for use in conducting base editing directly on mtDNA, i.e., on a double-stranded DNA target.

### Cas9

The term “Cas9” or “Cas9 nuclease” refers to an RNA-guided nuclease comprising a Cas9 domain, or a fragment thereof (e.g., a protein comprising an active or inactive DNA cleavage domain of Cas9, and/or the gRNA binding domain of Cas9). A “Cas9 domain” as used herein, is a protein fragment comprising an active or inactive cleavage domain of Cas9 and/or the gRNA binding domain of Cas9. A “Cas9 protein” is a full length Cas9 protein. A Cas9 nuclease is also referred to sometimes as a casnI nuclease or a CRISPR (Clustered Regularly Interspaced Short Palindromic Repeat)-associated nuclease. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 domain. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the entire contents of which are hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease comprises one or more mutations that partially impair or inactivate the DNA cleavage domain.

A nuclease-inactivated Cas9 domain may interchangeably be referred to as a “dCas9” protein (for nuclease-“dead” Cas9). Methods for generating a Cas9 domain (or a fragment thereof) having an inactive DNA cleavage domain are known (see, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)). In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, at least about 99.8% identical, or at least about 99.9% identical to wild type Cas9 (e.g., SpCas9 of SEQ ID NO: 59). In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to wild type Cas9 (e.g., SpCas9 of SEQ ID NO: 59). In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9 (e.g., SpCas9 of SEQ ID NO: 59). In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SpCas9 of SEQ ID NO: 59).

As used herein, the term “nCas9” or “Cas9 nickase” refers to a Cas9 or a variant thereof, which cleaves or nicks only one of the strands of a target cut site thereby introducing a nick in a double strand DNA molecule rather than creating a double strand break. This can be achieved by introducing appropriate mutations in a wild-type Cas9 which inactivates one of the two endonuclease activities of the Cas9. Any suitable mutation which inactivates one Cas9 endonuclease activity but leaves the other intact is contemplated, such as one of D10A or H840A mutations in the wild-type S. pyogenes Cas9 amino acid sequence, or a D10A mutation in the wild-type S. aureus Cas9 amino acid sequence, may be used to form the nCas9.

### Cytidine Deaminase

As used herein, a “cytidine deaminase” encoded by the CDA gene is an enzyme that catalyzes the removal of an amine group from cytidine (i.e., the base cytosine when attached to a ribose ring) to uridine (C to U) and deoxycytidine to deoxyuridine (C to U). A non-limiting example of a cytidine deaminase is APOBEC1 (“apolipoprotein B mRNA editing enzyme, catalytic polypeptide 1”). Another example is AID (“activation-induced cytidine deaminase”). Under standard Watson-Crick hydrogen bond pairing, a cytosine base hydrogen bonds to a guanine base. When cytidine is converted to uridine (or deoxycytidine is converted to deoxyuridine), the uridine (or the uracil base of uridine) undergoes hydrogen bond pairing with the base adenine. Thus, a conversion of “C” to uridine (“U”) by cytidine deaminase will cause the insertion of “A” instead of a “G” during cellular repair and/or replication processes. Since the adenine “A” pairs with thymine “T”, the cytidine deaminase in coordination with DNA replication causes the conversion of an C-G pairing to a T. A pairing in the double-stranded DNA molecule.

### Deaminase

The term “deaminase” or “deaminase domain” refers to a protein or enzyme that catalyzes a deamination reaction. In some embodiments, the deaminase is an adenosine (or adenine) deaminase, which catalyzes the hydrolytic deamination of adenine or adenosine. In some embodiments, the adenosine deaminase catalyzes the hydrolytic deamination of adenine or adenosine in deoxyribonucleic acid (DNA) to inosine. In other embodiments, the deaminase is a cytidine (or cytosine) deaminase, which catalyzes the hydrolytic deamination of cytidine or cytosine. In preferred aspects, the deaminase is a double-stranded DNA deaminase, or is modified, evolved, or otherwise altered to be able to utilize double-strand DNA as a substrate for deamination.

The deaminase embraces the DddA domains described herein, and defined below. The DddA is a type of deaminase, but where the activity of the deaminase is against double-stranded DNA, rather than single-stranded DNA, which is the case for deaminases prior to the present disclosure.

The deaminases provided herein may be from any organism, such as a bacterium. In some embodiments, the deaminase or deaminase domain is a variant of a naturally-occurring deaminase from an organism. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase.

### DNA Editing Efficiency

The term “DNA editing efficiency,” as used herein, refers to the number or proportion of intended base pairs that are edited. For example, if a base editor edits 10% of the base pairs that it is intended to target (e.g., within a cell or within a population of cells), then the base editor can be described as being 10% efficient. Some aspects of editing efficiency embrace the modification (e.g. deamination) of a specific nucleotide within DNA, without generating a large number or percentage of insertions or deletions (i.e., indels). It is generally accepted that editing while generating less than 5% indels (as measured over total target nucleotide substrates) is high editing efficiency. The generation of more than 20% indels is generally accepted as poor or low editing efficiency. Indel formation may be measured by techniques known in the art, including high-throughput screening of sequencing reads.

### DddA and DddA Variants (or Evolved DddAs)

The term “double-stranded DNA deaminase domain” or “DddA” (or equivalently, DddE) refers to a protein which catalyzes a deamination of a target nucleotide (e.g., C, A, G, C) in a double-stranded DNA molecule. Reference to DddA and double-stranded DNA deaminase are equivalent. In one embodiment, the DddA deaminates a cytidine. Deamination of cytidine, results in a uracil (or deoxyuracil in the case of deoxycytidine), and through replication and/or repair processes, converts the original C:G base pair to a T:A base pair. This change can also be referred to as a “C-to-T” edit because the C of the C:G pair is converted to a T of T:A pair. DddA, when expressed naturally, can be toxic to biological systems. While the mechanism of action is not clearly documented, one rationale for the observed toxicity is DddA's activity may cause indiscriminate deamination of cytidine in vivo on double-stranded target DNA (e.g., the cellular genome). Such indiscriminate deaminations may provoke cellular repair responses, including, but not limited to, degradation of genomic DNA. Herein described are variants of canonical DddA or “evolved DddA” variants or proteins. Canonical DddA was described in Mok et al., “A bacterial cytidine deaminase toxin enables CRISPR-free mitochondrial base editing,” Nature, 2020; 583(7817): 631-637 (“Mok et al., 2020”), (incorporated herein by reference). Canonical DddA was discovered in Burkholderia cenocepia and reported Mok et al. and in the Protein Data Bank as PDB ID: 6U08, which has the following full-length amino acid sequence (1427 amino acids):

>tr|A0A1V6L4E7|A0A1V6L4E7_9BURK YD repeat (Two copies) OS = Burkholderia
cenocepacia OX = 95486 GN = UE95_03830 PE = 1 SV = 1 (1427 AA the canonical
protein or “canonical DddA”)
(SEQ ID NO: 16)
MYEAARVTDPIDHTSALAGFLVGAVLGIALIAAVAFATFTCGFGVALLAGMMAGIGAQALLSIGESIG
KMFSSQSGNIITGSPDVYVNSLSAAYATLSGVACSKHNPIPLVAQGSTNIFINGRPAARKDDKITCGATI
GDGSHDTFFHGGTQTYLPVDDEVPPWLRTATDWAFTLAGLVGGLGGLLKASGGLSRAVLPCAAKFIG
GYVLGEAFGRYVAGPAINKAIGGLFGNPIDVTTGRKILLAESETDYVIPSPLPVAIKRFYSSGIDYAGTL
GRGWVLPWEIRLHARDGRLWYTDAQGRESGFPMLRAGQAAFSEADQRYLTRTPDGRYILHDLGERY
YDFGQYDPESGRIAWVRRVEDQAGQWYQFERDSRGRVTEILTCGGLRAVLDYETVFGRLGTVTLVH
EDERRLAVTYGYDENGQLASVTDANGAVVRQFAYTNGLMTSHMNALGFTSSYVWSKIEGEPRVVET
HTSEGENWTFEYDVAGRQTRVRHADGRTAHWRFDAQSQIVEYTDLDGAFYRIKYDAVGMPVMLML
PGDRTVMFEYDDAGRIIAETDPLGRTTRTRYDGNSLRPVEVVGPDGGAWRVEYDQQGRVVSNQDSL
GRENRYEYPKALTALPSAHIDALGGRKTLEWNSLGKLVGYTDCSGKTTRTSFDAFGRICSRENALGQR
ITYDVRPTGEPRRVTYPDGSSETFEYDAAGTLVRYIGLGGRVQELLRNARGQLIEAVDPAGRRVQYRY
DVEGRLRELQQDHARYTFTYSAGGRLLTETRPDGILRRFEYGEAGELLGLDIVGAPDPHATGNRSVRT
IRFERDRMGVLKVQRTPTEVTRYQHDKGDRLVKVERVPTPSGIALGIVPDAVEFEYDKGGRLVAEHG
SNGSVIYTLDELDNVVSLGLPHDQTLQMLRYGSGHVHQIRFGDQVVADFERDDLHREVSRTQGRLTQ
RSGYDPLGRKVWQSAGIDPEMLGRGSGQLWRNYGYDAAGDLIETSDSLRGSTRFSYDPAGRLISRAN
PLDRKFEEFAWDAAGNLLDDAQRKSRGYVEGNRLLMWQDLRFEYDPFGNLATKRRGANQTQRFTY
DGQDRLITVHTQDVRGVVETRFAYDPLGRRIAKTDTAFDLRGMKLRAETKRFVWEGLRLVQEVRET
GVSSYVYSPDAPYSPVARADTVMAEALAATVIDSAKRAARIFHFHTDPVGAPQEVTDEAGEVAWAG
QYAAWGKVEATNRGVTAARTDQPLRFAGQYADDSTGLHYNTFRFYDPDVGRFINQDPIGLNGGANV
YHYAPNPVGWVDPWGLAGSYALGPYQISAPQLPAYNGQTVGTFYYVNDAGGLESKVFSSGGPTPYP
NYANAGHVEGQSALFMRDNGISEGLVFHNNPEGTCGFCVNMTETLLPENAKMTVVPPEGAIPVKRGA
TGETKVFTGNSNSPKSPTKGGC.

As reported in Mok et al. 2020, amino acids 1264-1427 of DddA were identified as the domain that conferred toxicity, i.e., referred to as “DddAₜₒₓ” or the toxin domain.

### Effective Amount

The term “effective amount,” as used herein, refers to an amount of a biologically active agent that is sufficient to elicit a desired biological response. For example, in some embodiments, an effective amount of any of the fusion proteins as described herein, or compositions thereof, may refer to the amount of the fusion proteins sufficient to edit a target nucleotide sequence (e.g., mtDNA). In some embodiments, an effective amount of any of the fusion proteins as described herein, or compositions thereof (e.g., a fusion protein comprising a first mitoTALE or another pDNAbp and a first portion of a DddA, a second fusion protein comprising a second mitoTALE or another pDNAbp and a second portion of a DddA) that is sufficient to induce editing of a target nucleotide, which is proximal to a target nucleic acid sequence specifically bound and edited by the fusion protein (e.g., by the first or second mitoTALE). As will be appreciated by the skilled artisan, the effective amount of an agent (e.g., a fusion protein, a second fusion protein), may vary depending on various factors as, for example, on the desired biological response on the specific allele, genome, or target site to be edited, on the cell or tissue being targeted, and on the agent being used.

### Fusion Protein

The term “fusion protein” as used herein refers to a hybrid polypeptide which comprises protein domains from at least two different proteins (e.g., a first mitoTALE, a first portion of a DddA, a second mitoTALE, a second portion of a DddA). One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding site (e.g., a first or second mitoTALE) and a catalytic domain of a nucleic-acid editing protein (e.g., a first or second portion of a DddA). Another example includes a mitoTALE to a DddA or portion thereof. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

### Guide Nucleic Acid

In certain embodiments, the PACE-evolved DddA variants can be fused to an nucleic acid-programmable DNA binding protein (“napDNAbp”), such as Cas9. In such embodiments, the Cas9 domain requires a guide RNA (or more generically, a guide nucleic acid) to program the binding of the Cas9 to a target site. The term “guide nucleic acid” or “napDNAbp-programming nucleic acid molecule” or equivalently “guide sequence” refers the one or more nucleic acid molecules which associate with and direct or otherwise program a napDNAbp protein to localize to a specific target nucleotide sequence (e.g., a gene locus of a genome) that is complementary to the one or more nucleic acid molecules (or a portion or region thereof) associated with the protein, thereby causing the napDNAbp protein to bind to the nucleotide sequence at the specific target site. A non-limiting example is a guide RNA of a Cas protein of a CRISPR-Cas genome editing system.

Guide RNA is a particular type of guide nucleic acid which is mostly commonly associated with a Cas protein of a CRISPR-Cas9 and which associates with Cas9, directing the Cas9 protein to a specific sequence in a DNA molecule that includes complementarity to protospacer sequence of the guide RNA. As used herein, a “guide RNA” refers to a synthetic fusion of the endogenous bacterial crRNA and tracrRNA that provides both targeting specificity and scaffolding and/or binding ability for Cas9 nuclease to a target DNA. This synthetic fusion does not exist in nature and is also commonly referred to as an sgRNA. However, this term also embraces the equivalent guide nucleic acid molecules that associate with Cas9 equivalents, homologs, orthologs, or paralogs, whether naturally occurring or non-naturally occurring (e.g., engineered or recombinant), and which otherwise program the Cas9 equivalent to localize to a specific target nucleotide sequence. The Cas9 equivalents may include other napDNAbp from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353(6299), the contents of which are incorporated herein by reference. Exemplary sequences are and structures of guide RNAs are provided herein. In addition, methods for designing appropriate guide RNA sequences are provided herein.

Guide RNA (“gRNA”)

In embodiments involving pDNAbp/DddA base editors that comprise Cas9 domains as the pDNAbp component, the Cas9 domain requires a guide RNA (or more generically, a guide nucleic acid) to program the binding of the Cas9 to a target site. As used herein, the term “guide RNA” is a particular type of guide nucleic acid which is mostly commonly associated with a Cas protein of a CRISPR-Cas9 and which associates with Cas9, directing the Cas9 protein to a specific sequence in a DNA molecule that includes complementarity to protospacer sequence of the guide RNA. However, this term also embraces the equivalent guide nucleic acid molecules that associate with Cas9 equivalents, homologs, orthologs, or paralogs, whether naturally occurring or non-naturally occurring (e.g., engineered or recombinant), and which otherwise program the Cas9 equivalent to localize to a specific target nucleotide sequence. The Cas9 equivalents may include other napDNAbp from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353(6299), the contents of which are incorporated herein by reference. Exemplary sequences are and structures of guide RNAs are provided herein.

Guide RNAs may comprise various structural elements that include, but are not limited to (a) a spacer sequence—the sequence in the guide RNA (having ˜20 nts in length) which binds to a complementary strand of the target DNA (and has the same sequence as the protospacer of the DNA) and (b) a gRNA core (or gRNA scaffold or backbone sequence)—refers to the sequence within the gRNA that is responsible for Cas9 binding, it does not include the ˜20 bp spacer sequence that is used to guide Cas9 to target DNA.

### Guide RNA Target Sequence

As used herein, the “guide RNA target sequence” refers to the ˜20 nucleotides that are complementary to the protospacer sequence in the PAM strand. The target sequence is the sequence that anneals to or is targeted by the spacer sequence of the guide RNA. The spacer sequence of the guide RNA and the protospacer have the same sequence (except the spacer sequence is RNA and the protospacer is DNA).

### Guide RNA Scaffold Sequence

As used herein, the “guide RNA scaffold sequence” refers to the sequence within the gRNA that is responsible for napDNAbp binding, it does not include the 20 bp spacer/targeting sequence that is used to guide napDNAbp to target DNA.

### Host Cell

The term “host cell,” as used herein, refers to a cell that can host, replicate, and transfer a phage vector useful for a continuous evolution process as provided herein. In embodiments where the vector is a viral vector, a suitable host cell is a cell that may be infected by the viral vector, can replicate it, and can package it into viral particles that can infect fresh host cells. A cell can host a viral vector if it supports expression of genes of viral vector, replication of the viral genome, and/or the generation of viral particles. One criterion to determine whether a cell is a suitable host cell for a given viral vector is to determine whether the cell can support the viral life cycle of a wild-type viral genome that the viral vector is derived from. For example, if the viral vector is a modified M13 phage genome, as provided in some embodiments described herein, then a suitable host cell would be any cell that can support the wild-type M13 phage life cycle. Suitable host cells for viral vectors useful in continuous evolution processes are well known to those of skill in the art, and the disclosure is not limited in this respect. In some embodiments, the viral vector is a phage and the host cell is a bacterial cell. In some embodiments, the host cell is an E. coli cell. Suitable E. coli host strains will be apparent to those of skill in the art, and include, but are not limited to, New England Biolabs (NEB) Turbo, Top10F′, DH12S, ER2738, ER2267, and XL1-Blue MRF′. These strain names are art recognized and the genotype of these strains has been well characterized. It should be understood that the above strains are exemplary only and that the invention is not limited in this respect. The term “fresh,” as used herein interchangeably with the terms “non-infected” or “uninfected” in the context of host cells, refers to a host cell that has not been infected by a viral vector comprising a gene of interest as used in a continuous evolution process provided herein. A fresh host cell can, however, have been infected by a viral vector unrelated to the vector to be evolved or by a vector of the same or a similar type but not carrying the gene of interest.

In some embodiments, the host cell is a prokaryotic cell, for example, a bacterial cell. In some embodiments, the host cell is an E. coli cell. In some embodiments, the host cell is a eukaryotic cell, for example, a yeast cell, an insect cell, or a mammalian cell. The type of host cell, will, of course, depend on the viral vector employed, and suitable host cell/viral vector combinations will be readily apparent to those of skill in the art.

### Inteins and Split-Inteins

In some embodiments, the Evolved DddA-containing base editors or the polypeptides that comprise the Evolved DddA-containing base editors (e.g., the pDNAbps and DddA) may be engineered to include intein and/or split-intein amino acid sequences.

As used herein, the term “intein” refers to auto-processing polypeptide domains found in organisms from all domains of life. An intein (intervening protein) carries out a unique auto-processing event known as protein splicing in which it excises itself out from a larger precursor polypeptide through the cleavage of two peptide bonds and, in the process, ligates the flanking extein (external protein) sequences through the formation of a new peptide bond. This rearrangement occurs post-translationally (or possibly co-translationally), as intein genes are found embedded in frame within other protein-coding genes. Furthermore, intein-mediated protein splicing is spontaneous; it requires no external factor or energy source, only the folding of the intein domain. This process is also known as cis-protein splicing, as opposed to the natural process of trans-protein splicing with “split inteins.”

Split inteins are a sub-category of inteins. Unlike the more common contiguous inteins, split inteins are transcribed and translated as two separate polypeptides, the N-intein and C-intein, each fused to one extein. Upon translation, the intein fragments spontaneously and non-covalently assemble into the canonical intein structure to carry out protein splicing in trans.

Inteins and split inteins are the protein equivalent of the self-splicing RNA introns (see Perler et al., Nucleic Acids Res. 22:1125-1127 (1994)), which catalyze their own excision from a precursor protein with the concomitant fusion of the flanking protein sequences, known as exteins (reviewed in Perler et al., Curr. Opin. Chem. Biol. 1:292-299 (1997); Perler, F. B. Cell 92(1):1-4 (1998); Xu et al., EMBO J. 15(19):5146-5153 (1996)).

As used herein, the term “protein splicing” refers to a process in which an interior region of a precursor protein (an intein) is excised and the flanking regions of the protein (exteins) are ligated to form the mature protein. This natural process has been observed in numerous proteins from both prokaryotes and eukaryotes (Perler, F. B., Xu, M. Q., Paulus, H. Current Opinion in Chemical Biology 1997, 1, 292-299; Perler, F. B. Nucleic Acids Research 1999, 27, 346-347). The intein unit contains the necessary components needed to catalyze protein splicing and often contains an endonuclease domain that participates in intein mobility (Perler, F. B., Davis, E. O., Dean, G. E., Gimble, F. S., Jack, W. E., Neff, N., Noren, C. J., Thomer, J., Belfort, M. Nucleic Acids Research 1994, 22, 1127-1127). The resulting proteins are linked, however, not expressed as separate proteins. Protein splicing may also be conducted in trans with split inteins expressed on separate polypeptides spontaneously combine to form a single intein which then undergoes the protein splicing process to join to separate proteins.

The elucidation of the mechanism of protein splicing has led to a number of intein-based applications (Comb, et al., U.S. Pat. No. 5,496,714; Comb, et al., U.S. Pat. No. 5,834,247; Camarero and Muir, J. Amer. Chem. Soc., 121:5597-5598 (1999); Chong, et al., Gene, 192:271-281 (1997), Chong, et al., Nucleic Acids Res., 26:5109-5115 (1998); Chong, et al., J. Biol. Chem., 273:10567-10577 (1998); Cotton, et al. J. Am. Chem. Soc., 121:1100-1101 (1999); Evans, et al., J. Biol. Chem., 274:18359-18363 (1999); Evans, et al., J. Biol. Chem., 274:3923-3926 (1999); Evans, et al., Protein Sci., 7:2256-2264 (1998); Evans, et al., J. Biol. Chem., 275:9091-9094 (2000); Iwai and Pluckthun, FEBS Lett. 459:166-172 (1999); Mathys, et al., Gene, 231:1-13 (1999); Mills, et al., Proc. Natl. Acad. Sci. USA 95:3543-3548 (1998); Muir, et al., Proc. Natl. Acad. Sci. USA 95:6705-6710 (1998); Otomo, et al., Biochemistry 38:16040-16044 (1999); Otomo, et al., J. Biolmol. NMR 14:105-114 (1999); Scott, et al., Proc. Natl. Acad. Sci. USA 96:13638-13643 (1999); Severinov and Muir, J. Biol. Chem., 273:16205-16209 (1998); Shingledecker, et al., Gene, 207:187-195 (1998); Southworth, et al., EMBO J. 17:918-926 (1998); Southworth, et al., Biotechniques, 27:110-120 (1999); Wood, et al., Nat. Biotechnol., 17:889-892 (1999); Wu, et al., Proc. Natl. Acad. Sci. USA 95:9226-9231 (1998a); Wu, et al., Biochim Biophys Acta 1387:422-432 (1998b); Xu, et al., Proc. Natl. Acad. Sci. USA 96:388-393 (1999); Yamazaki, et al., J. Am. Chem. Soc., 120:5591-5592 (1998)). Each reference is incorporated herein by reference.

### Lentiviral Vectors

Lentiviral vectors are derived from human immunodeficiency virus-1 (HIV-1). The lentiviral genome consists of single-stranded RNA that is reverse-transcribed into DNA and then integrated into the host cell genome. Lentiviruses can infect both dividing and non-dividing cells, making them attractive tools for gene therapy.

The lentiviral genome is around 9 kb in length and contains three major structural genes: gag, pol, and env. The gag gene is translated into three viral core proteins: 1) matrix (MA) proteins, which are necessary for virion assembly and infection of non-dividing cells; 2) capsid (CA) proteins, which form the hydrophobic core of the virion; and 3) nucleocapsid (NC) proteins, which protect the viral genome by coating and associating tightly with the RNA. The pol gene encodes for the viral protease, reverse transcriptase, and integrase enzymes which are essential for viral replication. The env gene encodes for the viral surface glycoproteins, which are essential for virus entry into the host cell by enabling binding to cellular receptors and fusion with cellular membranes. In some embodiments, the viral glycoprotein is derived from vesicular stomatitis virus (VSV-G). The viral genome also contains regulatory genes, including tat and rev. Tat encodes transactivators critical for activating viral transcription, while rev encodes a protein that regulates the splicing and export of viral transcripts. Tat and rev are the first proteins synthesized following viral integration and are required to accelerate production of viral mRNAs.

To improve the safety of lentivirus, the components necessary for viral production are split across multiple vectors. In some embodiments, the disclosure relates to delivery of a heterologous gene (e.g., transgene) via a recombinant lentiviral transfer vector encoding one or more transgenes of interest flanked by long terminal repeat (LTR) sequences. These LTRs are identical nucleotide sequences that are repeated hundreds or thousands of times and facilitate the integration of the transfer plasmid sequences into the host cell genome. Methods of the current disclosure also describe one or more accessory plasmids. These accessory plasmids may include one or more lentiviral packaging plasmids, which encode the pol and rev genes that are necessary for the replication, splicing, and export of viral particles. The accessory plasmids may also include a lentiviral envelope plasmid, which encodes the genes necessary for producing the viral glycoproteins which will allow the viral particle to fuse with the host cell.

### Ligand-Dependent Intein

In some embodiments, the Evolved DddA-containing base editors or the polypeptides that comprise the Evolved DddA-containing base editors (e.g., the pDNAbps and DddA) may be engineered to include ligand-dependent inteins.

The term “ligand-dependent intein,” as used herein refers to an intein that comprises a ligand-binding domain. Typically, the ligand-binding domain is inserted into the amino acid sequence of the intein, resulting in a structure intein (N)-ligand-binding domain-intein (C). Typically, ligand-dependent inteins exhibit no or only minimal protein splicing activity in the absence of an appropriate ligand, and a marked increase of protein splicing activity in the presence of the ligand. In some embodiments, the ligand-dependent intein does not exhibit observable splicing activity in the absence of ligand but does exhibit splicing activity in the presence of the ligand. In some embodiments, the ligand-dependent intein exhibits an observable protein splicing activity in the absence of the ligand, and a protein splicing activity in the presence of an appropriate ligand that is at least 5 times, at least 10 times, at least 50 times, at least 100 times, at least 150 times, at least 200 times, at least 250 times, at least 500 times, at least 1000 times, at least 1500 times, at least 2000 times, at least 2500 times, at least 5000 times, at least 10000 times, at least 20000 times, at least 25000 times, at least 50000 times, at least 100000 times, at least 500000 times, or at least 1000000 times greater than the activity observed in the absence of the ligand. In some embodiments, the increase in activity is dose dependent over at least 1 order of magnitude, at least 2 orders of magnitude, at least 3 orders of magnitude, at least 4 orders of magnitude, or at least 5 orders of magnitude, allowing for fine-tuning of intein activity by adjusting the concentration of the ligand. Suitable ligand-dependent inteins are known in the art, and in include those provided below and those described in published U.S. Patent Application U.S. 2014/0065711 A1; Mootz et al., “Protein splicing triggered by a small molecule.” J. Am. Chem. Soc. 2002; 124, 9044-9045; Mootz et al., “Conditional protein splicing: a new tool to control protein structure and function in vitro and in vivo.” J. Am. Chem. Soc. 2003; 125, 10561-10569; Buskirk et al., Proc. Natl. Acad. Sci. USA. 2004; 101, 10505-10510); Skretas & Wood, “Regulation of protein activity with small-molecule-controlled inteins.” Protein Sci. 2005; 14, 523-532; Schwartz, et al., “Post-translational enzyme activation in an animal via optimized conditional protein splicing.” Nat. Chem. Biol. 2007; 3, 50-54; Peck et al., Chem. Biol. 2011; 18 (5), 619-630; the entire contents of each are hereby incorporated by reference. Exemplary sequences are as follows:

| | | SEQ ID |
|---|---|---|
| NAME | SEQUENCE OF LIGAND-DEPENDENT INTEIN | NO: |
| 2-4 | CLAEGTRIFDPVTGTTHRIEDVVDGRKPIHVVAAAKDGTLLARPVVSWFDQGTRDVIGLRIAGGAIVWATP | 17 |
| INTEIN: | DHKVLTEYGWRAAGELRKGDRVAGPGGSGNSLALSLTADQMVSALLDAEPPILYSEYDPTSPFSEASMMGL | |
| | LTNLADRELVHMINWAKRVPGFVDLTLHDQAHLLECAWLEILMIGLVWRSMEHPGKLLFAPNLLLDRNQG | |
| | KCVEGMVEIFDMLLATSSRFRMMNLQGEEFVCLKSIILLNSGVYTFLSSTLKSLEEKDHIHRALDKITDTLIH | |
| | LMAKAGLTLQQQHQRLAQLLLILSHIRHMSNKGMEHLYSMKYKNVVPLYDLLLEMLDAHRLHAGGSGAS | |
| | RVQAFADALDDKFLHDMLAEELRYSVIREVLPTRRARTFDLEVEELHTLVAEGVVVHNC | |
| 3-2 | CLAEGTRIFDPVTGTTHRIEDVVDGRKPIHVVAVAKDGTLLARPVVSWFDQGTRDVIGLRIAGGAIVWATPD | 18 |
| INTEIN | HKVLTEYGWRAAGELRKGDRVAGPGGSGNSLALSLTADQMVSALLDAEPPILYSEYDPTSPFSEASMMGLL | |
| | TNLADRELVHMINWAKRVPGFVDLTLHDQAHLLECAWLEILMIGLVWRSMEHPGKLLFAPNLLLDRNQGK | |
| | CVEGMVEIFDMLLATSSRFRMMNLQGEEFVCLKSIILLNSGVYTFLSSTLKSLEEKDHIHRALDKITDTLIHL | |
| | MAKAGLTLQQQHQRLAQLLLILSHIRHMSNKGMEHLYSMKYTNVVPLYDLLLEMLDAHRLHAGGSGASR | |
| | VQAFADALDDKFLHDMLAEELRYSVIREVLPTRRARTFDLEVEELHTLVAEGVVVHNC | |
| 30R3-1 | CLAEGTRIFDPVTGTTHRIEDVVDGRKPIHVVAAAKDGTLLARPVVSWFDQGTRDVIGLRIAGGATVWATP | 19 |
| INTEIN | DHKVLTEYGWRAAGELRKGDRVAGPGGSGNSLALSLTADQMVSALLDAEPPIPYSEYDPTSPFSEASMMGL | |
| | LTNLADRELVHMINWAKRVPGFVDLTLHDQAHLLECAWLEILMIGLVWRSMEHPGKLLFAPNLLLDRNQG | |
| | KCVEGMVEIFDMLLATSSRFRMMNLQGEEFVCLKSIILLNSGVYTFLSSTLKSLEEKDHIHRALDKITDTLIH | |
| | LMAKAGLTLQQQHQRLAQLLLILSHIRHMSNKGMEHLYSMKYKNVVPLYDLLLEMLDAHRLHAGGSGAS | |
| | RVQAFADALDDKFLHDMLAEGLRYSVIREVLPTRRARTFDLEVEELHTLVAEGVVVHNC | |
| 30R3-2 | CLAEGTRIFDPVTGTTHRIEDVVDGRKPIHVVAAAKDGTLLARPVVSWFDQGTRDVIGLRIAGGATVWATP | 20 |
| INTEIN | DHKVLTEYGWRAAGELRKGDRVAGPGGSGNSLALSLTADQMVSALLDAEPPILYSEYDPTSPFSEASMMGL | |
| | LTNLADRELVHMINWAKRVPGFVDLTLHDQAHLLECAWLEILMIGLVWRSMEHPGKLLFAPNLLLDRNQG | |
| | KCVEGMVEIFDMLLATSSRFRMMNLQGEEFVCLKSIILLNSGVYTFLSSTLKSLEEKDHIHRALDKITDTLIH | |
| | LMAKAGLTLQQQHQRLAQLLLILSHIRHMSNKGMEHLYSMKYKNVVPLYDLLLEMLDAHRLHAGGSGAS | |
| | RVQAFADALDDKFLHDMLAEELRYSVIREVLPTRRARTFDLEVEELHTLVAEGVVVHNC | |
| 30R3-3 | CLAEGTRIFDPVTGTTHRIEDVVDGRKPIHVVAAAKDGTLLARPVVSWFDQGTRDVIGLRIAGGATVWATP | 21 |
| INTEIN | DHKVLTEYGWRAAGELRKGDRVAGPGGSGNSLALSLTADQMVSALLDAEPPIPYSEYDPTSPFSEASMMGL | |
| | LTNLADRELVHMINWAKRVPGFVDLTLHDQAHLLECAWLEILMIGLVWRSMEHPGKLLFAPNLLLDRNQG | |
| | KCVEGMVEIFDMLLATSSRFRMMNLQGEEFVCLKSIILLNSGVYTFLSSTLKSLEEKDHIHRALDKITDTLIH | |
| | LMAKAGLTLQQQHQRLAQLLLILSHIRHMSNKGMEHLYSMKYKNVVPLYDLLLEMLDAHRLHAGGSGAS | |
| | RVQAFADALDDKFLHDMLAEELRYSVIREVLPTRRARTFDLEVEELHTLVAEGVVVHNC | |
| 37R3-1 | CLAEGTRIFDPVTGTTHRIEDVVDGRKPIHVVAAAKDGTLLARPVVSWFDQGTRDVIGLRIAGGATVWATP | 22 |
| INTEIN | DHKVLTEYGWRAAGELRKGDRVAGPGGSGNSLALSLTADQMVSALLDAEPPILYSEYNPTSPFSEASMMGL | |
| | LTNLADRELVHMINWAKRVPGFVDLTLHDQAHLLERAWLEILMIGLVWRSMEHPGKLLFAPNLLLDRNQG | |
| | KCVEGMVEIFDMLLATSSRFRMMNLQGEEFVCLKSIILLNSGVYTFLSSTLKSLEEKDHIHRALDKITDTLIH | |
| | LMAKAGLTLQQQHQRLAQLLLILSHIRHMSNKGMEHLYSMKYKNVVPLYDLLLEMLDAHRLHAGGSGAS | |
| | RVQAFADALDDKFLHDMLAEGLRYSVIREVLPTRRARTFDLEVEELHTLVAEGVVVHNC | |
| 37R3-2 | CLAEGTRIFDPVTGTTHRIEDVVDGRKPIHVVAAAKDGTLLARPVVSWFDQGTRDVIGLRIAGGAIVWATP | 23 |
| INTEIN | DHKVLTEYGWRAAGELRKGDRVAGPGGSGNSLALSLTADQMVSALLDAEPPILYSEYDPTSPFSEASMMGL | |
| | LTNLADRELVHMINWAKRVPGFVDLTLHDQAHLLERAWLEILMIGLVWRSMEHPGKLLFAPNLLLDRNQG | |
| | KCVEGMVEIFDMLLATSSRFRMMNLQGEEFVCLKSIILLNSGVYTFLSSTLKSLEEKDHIHRALDKITDTLIH | |
| | LMAKAGLTLQQQHQRLAQLLLILSHIRHMSNKGMEHLYSMKYKNVVPLYDLLLEMLDAHRLHAGGSGAS | |
| | RVQAFADALDDKFLHDMLAEGLRYSVIREVLPTRRARTFDLEVEELHTLVAEGVVVHNC | |
| 37R3-3 | CLAEGTRIFDPVTGTTHRIEDVVDGRKPIHVVAVAKDGTLLARPVVSWFDQGTRDVIGLRIAGGATVWATPD | 24 |
| INTEIN | HKVLTEYGWRAAGELRKGDRVAGPGGSGNSLALSLTADQMVSALLDAEPPILYSEYDPTSPFSEASMMGLL | |
| | TNLADRELVHMINWAKRVPGFVDLTLHDQAHLLERAWLEILMIGLVWRSMEHPGKLLFAPNLLLDRNQGK | |
| | CVEGMVEIFDMLLATSSRFRMMNLQGEEFVCLKSIILLNSGVYTFLSSTLKSLEEKDHIHRALDKITDTLIHL | |
| | MAKAGLTLQQQHQRLAQLLLILSHIRHMSNKGMEHLYSMKYKNVVPLYDLLLEMLDAHRLHAGGSGASR | |
| | VQAFADALDDKFLHDMLAEELRYSVIREVLPTRRARTFDLEVEELHTLVAEGVVVHNC | |

### Linker

In various embodiments, the herein disclosed fusion proteins (e.g., the evolved-DddA containing base editors) or the polypeptides that comprise the Evolved DddA-containing base editors (e.g., the pDNAbps and DddA) may be engineered to include one or more linker sequences that join two or more polypeptides (e.g., a pDNAbp and a DddA halt) to one another.

The term “linker,” as used herein, refers to a molecule linking two other molecules or moieties. The linker can be an amino acid sequence in the case of a linker joining two fusion proteins. For example, a first or second mitoTALE can be fused to a first or second portion of a DddA, by an amino acid linker sequence. The linker can also be a nucleotide sequence in the case of joining two nucleotide sequences together. In other embodiments, the linker is an organic molecule, group, polymer, or chemical moiety. In some embodiments, the linker is 1-100 amino acids in length, for example, 1, 2, 3,4, 5,6, 7, 8,9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer linkers are also contemplated. mitoTALE

In various embodiments, the Evolved DddA-containing base editors embrace fusion proteins comprising a DddA (or inactive fragment thereof) and a mitoTALE domain. As used herein, a “mitoTALE” protein or domain refers to a modified TALE protein that can be designed to localize to the mitochondria. In one embodiment, a mitoTALE comprises a TALE domain fused to a mitochondrial targeting sequences (MTS). In another embodiment, a mitoTALE comprises a TALE domain fused to an MTS in place of the endogenous LS (localization signal) of the TALE, or into the repeat variable diresidue (RVD) of the TALE. MTS domains can include, but are not limited to, SOD2, Cox8a, bipartite nuclear localization signals (BPNLS), zmLOC100282174 MLS), which are disclosed herein.

Transcription activator-like effector proteins (TALE proteins) are class of naturally occurring DNA binding proteins which bind specific promoter sequences and which can activate the expression of genes. TALE proteins can be engineered to recognize a desired DNA sequence. TALEs have a modular DNA-binding domain (DBD) consisting of repetitive sequences of amino acids with each repeat region comprising of 34 amino acids. The two amino acids at residue positions 12 and 13 of each repeat region determine the nucleotide specificity of the TALE. This pair of residues is referred to as the repeat variable diresidue (RVD). A final region, known as the half-repeat, is typically truncated to 20 amino acids. Using these factors, one of ordinary skill in the art can synthesize sequence-specific synthetic TALEs, which target user defined nucleotide sequences. See Garg A.; Lohmueller J. J.; Silver P. A.; Armel T. Z. (2012), “Engineering synthetic TAL effectors with orthogonal target sites,” Nucleic Acids Res. 40, 7584-7595, which is incorporated herein by reference. Further reference to designing sequence specific TALEs can be found in Carlson et al., “Targeting DNA with fingers and TALENs,” Mol. Ther. Nucleic Acids, 2012, 1, e3.10.1038/mtna.2011, which is incorporated herein by reference. For example, the C-terminus typically contains a localization signal (LS), which directs a TALE to the particular cellular component (e.g., mitochondria), as well as a functional domain that modulates transcription, such as an acidic activation domain (AD). The endogenous LS can be replaced by an organism-specific localization signal, such as a specific MLS to localize the TALE to the mitochondria. For example, an LS derived from the simian virus 40 large T-antigen can be used in mammalian cells.

### MitoZFP

In various embodiments, the Evolved DddA-containing base editors embrace fusion proteins comprising a DddA (or inactive fragment thereof) and a mitoZFP domain.

A “zinc finger DNA binding protein” or “ZFP” is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein can be abbreviated as zinc finger protein or ZFP. A “mitoZFP” refers to a zinc finger DNA binding protein that has been modified to comprise one or more mitochondrial targeting sequences (MTS).

Zinc finger binding domains can be “engineered” to bind to a predetermined nucleotide sequence. Non-limiting examples of methods for engineering zinc finger proteins are design and selection. A designed zinc finger protein is a protein not occurring in nature whose design/composition results principally from rational criteria. Rational criteria for design include application of substitution rules and computerized algorithms for processing information in a database storing information of existing ZFP designs and binding data. See, for example, U.S. Pat. Nos. 6,140,081; 6,453,242; 6,534,261; and 6,785,613; see, also WO 98/53058; WO 98/53059; WO 98/53060; WO 02/016536 and WO 03/016496; and U.S. Pat. Nos. 6,746,838; 6,866,997; and 7,030,215, each of which are incorporated herein by reference.

Zinc-finger nucleases (“ZFNs”) are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes.

The DNA-binding domains of individual ZFNs typically contain between three and six individual zinc finger repeats and can each recognize between 9 and 18 base pairs. If the zinc finger domains are perfectly specific for their intended target site then even a pair of 3-finger ZFNs that recognize a total of 18 base pairs can, in theory, target a single locus in a mammalian genome. The most straightforward method to generate new zinc-finger arrays is to combine smaller zinc-finger “modules” of known specificity. The most common modular assembly process involves combining three separate zinc fingers that can each recognize a 3 base pair DNA sequence to generate a 3-finger array that can recognize a 9 base pair target site.

### Mitochondrial Targeting Sequence (MTS)

In various embodiments, the Evolved DddA-containing base editors or the polypeptides that comprise the Evolved DddA-containing base editors (e.g., the pDNAbps and DddA) may be engineered to include one or more mitochondrial targeting sequences (MTS) (or mitochondrial localization sequence (MLS)) which facilitate that translocation of a polypeptide into the mitochondria. MTS are known in the art and exemplary sequences are provided herein. In general MTSs are short peptide sequences (about 3-70 amino acids long) that direct a newly synthesized protein to the mitochondria within a cell. It is usually found at the N-terminus and consists of an alternating pattern of hydrophobic and positively charged amino acids to form what is called an amphipathic helix. Mitochondrial localization sequences can contain additional signals that subsequently target the protein to different regions of the mitochondria, such as the mitochondrial matrix. One exemplary mitochondrial localization sequence is the mitochondrial localization sequence derived from Cox8, a mitochondrial cytochrome c oxidase subunit VIII. In embodiments, a mitochondrial localization sequence derived from Cox8 includes the amino acid sequence: MSVLTPLLLRGLTGSARRLPVPRAKIHSL (SEQ ID NO: 14). In the embodiments, the mitochondrial localization sequence derived from Cox8 includes an amino acid sequence that is about 50%, 55%, 60%, 65%, 70%, 75%, 80%, 85%, 90% or 95% identity to SEQ ID NO: 14.

### Nucleic Acid Molecule

The term “nucleic acid,” as used herein, refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5 bromouridine, C5 fluorouridine, C5 iodouridine, C5 propynyl uridine, C5 propynyl cytidine, C5 methylcytidine, 7 deazaadenosine, 7 deazaguanosine, 8 oxoadenosine, 8 oxoguanosine, O(6) methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′ N phosphoramidite linkages).

### Mutation

The term “mutation,” as used herein, refers to a substitution of a residue within a sequence, e.g. a nucleic acid or amino acid sequence, with another residue; a deletion or insertion of one or more residues within a sequence; or a substitution of a residue within a sequence of a genome in a subject to be corrected. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which are mutations that reduce or abolish a protein activity. Most loss-of-function mutations are recessive, because in a heterozygote the second chromosome copy carries an unmutated version of the gene coding for a fully functional protein whose presence compensates for the effect of the mutation. There are some exceptions where a loss-of-function mutation is dominant, one example being haploinsufficiency, where the organism is unable to tolerate the approximately 50% reduction in protein activity suffered by the heterozygote. This is the explanation for a few genetic diseases in humans, including Marfan syndrome, which results from a mutation in the gene for the connective tissue protein called fibrillin. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. For example, a mutation might lead to one or more genes being expressed in the wrong tissues, these tissues gaining functions that they normally lack. Alternatively, the mutation could lead to overexpression of one or more genes involved in control of the cell cycle, thus leading to uncontrolled cell division and hence to cancer. Because of their nature, gain-of-function mutations are usually dominant.

### NapDNAbp

In various embodiments, the Evolved DddA-containing base editors may comprise pDNAbps which are nucleic acid programmable. The term “napDNAbp” which stand for “nucleic acid programmable DNA binding protein” refers to any protein that may associate (e.g., form a complex) with one or more nucleic acid molecules (i.e., which may broadly be referred to as a “napDNAbp-programming nucleic acid molecule” and includes, for example, guide RNA in the case of Cas systems) which direct or otherwise program the protein to localize to a specific target nucleotide sequence (e.g., a gene locus of a genome) that is complementary to the one or more nucleic acid molecules (or a portion or region thereof) associated with the protein, thereby causing the protein to bind to the nucleotide sequence at the specific target site. This term napDNAbp embraces CRISPR-Cas9 proteins, as well as Cas9 equivalents, homologs, orthologs, or paralogs, whether naturally occurring or non-naturally occurring (e.g., engineered or modified), and may include a Cas9 equivalent from any type of CRISPR system (e.g., type II, V, VI), including Cpf1 (a type-V CRISPR-Cas systems), C2c1 (a type V CRISPR-Cas system), C2c2 (a type VI CRISPR-Cas system), C2c3 (a type V CRISPR-Cas system), dCas9, GeoCas9, CjCas9, Cas12a, Cas12b, Cas12c, Cas12d, Cas12g, Cas12h, Cas12i, Cas13d, Cas14, Argonaute, and nCas9. Further Cas-equivalents are described in Makarova et al., “C2c2 is a single-component programmable RNA-guided RNA-targeting CRISPR effector,” Science 2016; 353 (6299), the contents of which are incorporated herein by reference. However, the nucleic acid programmable DNA binding protein (napDNAbp) that may be used in connection with this invention are not limited to CRISPR-Cas systems. The invention embraces any such programmable protein, such as the Argonaute protein from Natronobacterium gregoryi (NgAgo) which may also be used for DNA-guided genome editing. NgAgo-guide DNA system does not require a PAM sequence or guide RNA molecules, which means genome editing can be performed simply by the expression of generic NgAgo protein and introduction of synthetic oligonucleotides on any genomic sequence. See Gao et al., DNA-guided genome editing using the Natronobacterium gregoryi Argonaute. Nature Biotechnology 2016; 34(7):768-73, which is incorporated herein by reference.

In some embodiments, the napDNAbp is a RNA-programmable nuclease, when in a complex with an RNA, may be referred to as a nuclease:RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 (or equivalent) complex to the target); and (2) a domain that binds a Cas9 protein. In some embodiments, domain (2) corresponds to a sequence known as a tracrRNA, and comprises a stem-loop structure. For example, in some embodiments, domain (2) is homologous to a tracrRNA as depicted in FIG. 1E of Jinek et al., Science 337:816-821(2012), the entire contents of which is incorporated herein by reference. Other examples of gRNAs (e.g., those including domain 2) can be found in U.S. Pat. No. 9,340,799, entitled “mRNA-Sensing Switchable gRNAs,” and International Patent Application No. PCT/US2014/054247, filed Sep. 6, 2013, published as WO 2015/035136 and entitled “Delivery System For Functional Nucleases,” the entire contents of each are herein incorporated by reference. In some embodiments, a gRNA comprises two or more of domains (1) and (2), and may be referred to as an “extended gRNA.” For example, an extended gRNA will, e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example Cas9 (Csn1) from Streptococcus pyogenes (see, e.g., “Complete genome sequence of an M1 strain of Streptococcus pyogenes.” Ferretti J. J. et al., Proc. Natl. Acad. Sci. U.S.A. 98:4658-4663(2001); “CRISPR RNA maturation by trans-encoded small RNA and host factor RNase III.” Deltcheva E. et al., Nature 471:602-607(2011); and “A programmable dual-RNA-guided DNA endonuclease in adaptive bacterial immunity.” Jinek M. et al., Science 337:816-821(2012), the entire contents of each of which are incorporated herein by reference.

The napDNAbp nucleases (e.g., Cas9) use RNA:DNA hybridization to target DNA cleavage sites, these proteins are able to be targeted, in principle, to any sequence specified by the guide RNA. Methods of using napDNAbp nucleases, such as Cas9, for site-specific cleavage (e.g., to modify a genome) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang, W. Y. et al. Efficient genome editing in zebrafish using a CRISPR-Cas system. Nature Biotechnology 31, 227-229 (2013); Jinek, M. et al. RNA-programmed genome editing in human cells. eLife 2, e00471 (2013); Dicarlo, J. E. et al., Genome engineering in Saccharomyces cerevisiae using CRISPR-Cas systems. Nucleic Acid Res. (2013); Jiang, W. et al. RNA-guided editing of bacterial genomes using CRISPR-Cas systems. Nature Biotechnology 31, 233-239 (2013); the entire contents of each of which are incorporated herein by reference).

### Nickase

The term “nickase” refers to a napDNAbp having only a single nuclease activity that cuts only one strand of a target DNA, rather than both strands. Thus, a nickase type napDNAbp does not leave a double-strand break.

### Nuclear Localization Signal

In various embodiments, the Evolved DddA-containing base editors or the polypeptides that comprise the Evolved DddA-containing base editors (e.g., the pDNAbps and DddA) may be further engineered to include one or more nuclear localization signals.

A nuclear localization signal or sequence (NLS) is an amino acid sequence that tags, designates, or otherwise marks a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus. Thus, a single nuclear localization signal can direct the entity with which it is associated to the nucleus of a cell. Such sequences may be of any size and composition, for example more than 25, 25, 15, 12, 10, 8, 7, 6, 5, or 4 amino acids, but will preferably comprise at least a four to eight amino acid sequence known to function as a nuclear localization signal (NLS).

### Nucleic Acid Molecule

The term “nucleic acid molecule” as used herein, refers to RNA as well as single and/or double-stranded DNA. Nucleic acid molecules may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g. a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g. analogs having other than a phosphodiester backbone. Nucleic acids may be purified from natural sources, produced using recombinant expression systems and optionally purified, chemically synthesized, etc. Where appropriate, e.g. in the case of chemically synthesized molecules, nucleic acids may comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g. 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, inosinedenosine, 8-oxoguanosine, 0(6)-methylguanine, and 2-thiocytidine); chemically modified bases; biologically modified bases (e.g. methylated bases); intercalated bases; modified sugars (e.g. 2′-fluororibose, ribose, 2′-deoxyribose, arabinose, and hexose); and/or modified phosphate groups (e.g. phosphorothioates and 5′-N-phosphoramidite linkages).

### PACE and PANCE

The term “phage-assisted continuous evolution (PACE),” as used herein, refers to continuous evolution that employs phage as viral vectors and is described in Thuronyi, B. W. et al. Nat Biotechnol 37, 1070-1079 (2019), the contents of which are incorporated herein by reference in their entirety. The general concept of PACE technology has also been described, for example, in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Application, U.S. Pat. No. 9,023,594, issued May 5, 2015, International PCT Application, PCT/US2015/012022, filed Jan. 20, 2015, published as WO 2015/134121 on Sep. 11, 2015, and International PCT Application, PCT/US2016/027795, filed Apr. 15, 2016, published as WO 2016/168631 on Oct. 20, 2016, the entire contents of each of which are incorporated herein by reference. PACE can be used, for instance, to evolve a deaminase (e.g., a cytidine or adenosine deaminase) which uses single strand DNA as a substrate to obtain a deaminase which is capable of using double-strand DNA as a substrate (e.g., DddA).

Variant Cas9s may also be obtain by phage-assisted non-continuous evolution (PANCE), which as used herein, refers to non-continuous evolution that employs phage as viral vectors. PANCE is a simplified technique for rapid in vivo directed evolution using serial flask transfers of evolving ‘selection phage’ (SP), which contain a gene of interest to be evolved, across fresh E. coli host cells, thereby allowing genes inside the host E. coli to be held constant while genes contained in the SP continuously evolve. Serial flask transfers have long served as a widely-accessible approach for laboratory evolution of microbes, and, more recently, analogous approaches have been developed for bacteriophage evolution. The PANCE system features lower stringency than the PACE system.

### Evolved DddA-Containing Base Editors

As used herein, the present disclosure describes use continuous evolution-based methods (e.g., PACE) to evolve DddA-containing base editors. In various embodiments, the evolved DddA can be linked to a programmable DNA binding protein (pDNAbp), which can include various such types of proteins, including but not limited to, TALE proteins, mitoTALE proteins (i.e., TALE proteins that specifically target mitochondria), zinc finger protein, and napDNAbps, such as Cas9. In principle, the evolved DddA-containing base editors may be used to edit any target double stranded DNA substrate in the cell, including in the cytoplasm, in the nucleus, or in an organelle such as a mitochondria. Preferably, when targeting mitochondrial DNA base editing, the evolved DddA-containing base editors comprise a mitoTALE or a zinc finger DNA binding protein. Amino acid sequences of exemplary evolved DddA-containing based editors and components thereof are provided herein, e.g., in XIII. Sequences.

Programmable DNA Binding Protein (pDNAbp)

As used herein, the term “programmable DNA binding protein,” “pDNA binding protein,” “pDNA binding protein domain” or “pDNAbp” refers to any protein that localizes to and binds a specific target DNA nucleotide sequence (e.g. a gene locus of a genome). This term embraces RNA-programmable proteins, which associate (e.g. form a complex) with one or more nucleic acid molecules (i.e., which includes, for example, guide RNA in the case of Cas systems) that direct or otherwise program the protein to localize to a specific target nucleotide sequence (e.g., DNA sequence) that is complementary to the one or more nucleic acid molecules (or a portion or region thereof) associated with the protein. The term also embraces proteins which bind directly to nucleotide sequence in an amino acid-programmable manner, e.g., zinc finger proteins and TALE proteins. Exemplary RNA-programmable proteins are CRISPR-Cas9 proteins, as well as Cas9 equivalents, homologs, orthologs, or paralogs, whether naturally occurring or non-naturally occurring (e.g. engineered or modified), and may include a Cas9 equivalent from any type of CRISPR system (e.g. type II, V, VI), including Cpf1 (a type-V CRISPR-Cas systems), C2c1 (a type V CRISPR-Cas system), C2c2 (a type VI CRISPR-Cas system), C2c3 (a type V CRISPR-Cas system), dCas9, GeoCas9, CjCas9, Cas12a, Cas12b, Cas12c, Cas12d, Cas12g, Cas12h, Cas12i, Cas13d, Cas14, Argonaute, and nCas9. Further Cas-equivalents are described in Makarova et al., “C2c2 is a single-component programmable RNA-guided RNA-targeting CRISPR effector,” Science 2016; 353(6299), the contents of which are incorporated herein by reference. When targeting the editing of mitochondrial DNA, it if preferable that the DNA binding protein and/or the evolved DddA protein are configured with a mitochondrial signal sequence.

### Promoter

The term “promoter” is recognized in the art as referring to a nucleic acid molecule with a sequence recognized by the cellular transcription machinery and able to initiate transcription of a downstream (i.e., closer to or toward the 3′ end of the nucleic acid strand) gene. A promoter can be constitutively active, meaning that the promoter is always active in a given cellular context, or conditionally active, meaning that the promoter is only active in the presence of a specific condition. For example, a conditional promoter may only be active in the presence of a specific protein that connects a protein associated with a regulatory element in the promoter to the basic transcriptional machinery, or only in the absence of an inhibitory molecule. A subclass of conditionally active promoters are inducible promoters that require the presence of a small molecule “inducer” for activity. Examples of inducible promoters include, but are not limited to, arabinose-inducible promoters, Tet-on promoters, and tamoxifen-inducible promoters. A variety of constitutive, conditional, and inducible promoters are well known to the skilled artisan, and the skilled artisan will be able to ascertain a variety of such promoters useful in carrying out the instant invention, which is not limited in this respect.

### Protein, Peptide, and Polypeptide

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups {e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the njPAC-R7B Biochemical Nomenclature Commission. Nucleotides, likewise, may be referred to by their commonly accepted single-letter codes. The terms “polypeptide,” “peptide” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

### Split Site (e.g., of a DddA)

As used herein, the term “split site,” as in a split site of a DddA, refers to a specific peptide bond between any two immediately adjacent amino acid residues in the amino acid sequence of a DddA at which the complete DddA polypeptide is divided into two half portions, i.e., an N-terminal half portion and a C-terminal half portion. The N-terminal half portion of the DddA may be referred to as “DddA-N half” and the C-terminal half portion of the DddA may be referred to as the “DddA-C half.” Alternately, DddA-N half may be referred to as the “DddA-N fragment or portion” and the DddA-C half may be referred to as the “DddA-C fragment of portion.” Depending on the location of the split site, the DddA-N half and the DddA-C half may be the same or different size and/or sequence length. The term “half” does not connote the requirement that the DddA-N and DddA-C portions are identically half of the size and/or sequence length of a complete DddA, or that the split site is required to be at the mid-point of the complete DddA polypeptide. To the contrary, and as noted above, the split site can be between any pair of residues in the DddA polypeptide, thereby giving rise to half portions which are unequal in size and/or sequence length. For clarity, as used herein, the term “half” when used in the context of a split molecule (e.g., protein, intein, delivery molecule, nucleic acid, etc.), shall not be interpreted to require, and shall not imply, that the size of the resulting portions (e.g., as “split” or broken into smaller portions) of the molecule are one-half (e.g., ½, 50%) of the original molecule. The term shall be interpreted to be illustrative of idea that they are portion(s) of a larger molecule that has been broken into smaller fragments (e.g., portions), but that when reconstituted may regain the activity of the molecule as a whole. Thus, by way of example, a half (e.g., portion) may be any portion of the molecule from which it is obtained (e.g., is less than 100% of the whole of the molecule), such that there is at least one additional portion formed (e.g., a second half, other half, second portion), which also is less than 100% of the whole of the molecule. It is important to note, that the molecule may be formed into additional portions (e.g., third, fourth, etc., halves (e.g., portions)), which is readily envisioned by using the term definition above, and such additional halves to not constitute a molecule larger than or in addition to the whole from which they were derived. Further, it should be noted that in the event there are more than two halves (e.g., two portions) formed from the splitting of a molecule it may only require two of the portions to reconstitute the activity of the molecule as a whole. By way of example, if an enzyme is split into three halves (e.g., three portions), wherein the catalytic domain of the enzyme possessing the enzymatic activity of interest is only split into two halves (e.g., two portions) only the two portions of the catalytic domain may be necessary to be used to carry out the activity of interest. Thus, when referring to using two halves, it is not necessary that the two halves, together, comprise 100% of the whole of the molecule from which they were derived. In certain embodiments, the split site is within a loop region of the DddA.

As used herein, reference to “splitting a DddA at a split site” embraces direct and indirect means for obtaining two half portions of a DddA. In one embodiment, splitting a DddA refers to the direct splitting a DddA polypeptide at a split site in the protein to obtain the DddA-N and DddA-C half portions. For example, the cleaving of a peptide bond between two adjacent amino acid residues at a split site may be achieved by enzymatic or chemical means. In another embodiment, a DddA may be split by engineering separate nucleic acid sequences, each encoding a different half portion of the DddA. Such methods can be used to obtain expression vectors for expressing the DddA half portions in a cell in order to reconstitute the DddA.

Exemplary split sites include G1333 and G1397. The nomenclature “G1333” refers to a split corresponding to the peptide bond between residues 1333 and 1334 of the canonical DddA protein. Similarly, “G1397” refers to a split corresponding to the peptide bond between residues 1397 and 1398. Thus, in reference to a DddA split at G1333, the N-terminal half of DddA would include the G residue. Similarly, in reference to a DddA split at G1397, the N-terminal half of DddA would include the G residue.

Given that the activity of canonical DddA has a cytidine deaminase activity, the base editor system involving split DddA domains (i.e., an N-terminal and a C-terminal half) each fused to a programmable binding domain that is programmed to bind to either side of a target site of deamination (i.e., a target cytidine), can be referred to as a “DdCBE” or double-stranded DNA cytidine base editor. Alternatively, the base editors disclosed herein may be referred to as evolved DddA-containing base editors because they comprise evolved DddA domains.

### Subject

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development.

### Target Site

The term “target site” refers to a sequence within a nucleic acid molecule (e.g., a mtDNA) that is edited by an evolved DddA-containing base editor disclosed herein. The target site further refers to the sequence within a nucleic acid molecule to which a complex of the evolved-DddA containing base editor binds. In cases wherein the pDNAbp of the evolved-DddA containing base editor is a Cas9 domain, typically, the target site is a sequence that includes the unique ˜20 bp target specified by the gRNA plus the genomic PAM sequence. CRISPR-Cas9 mechanisms recognize DNA targets that are complementary to a short CRISPR sgRNA sequence. The part of the sgRNA sequence that is complementary to the target sequence is known as a protospacer. In order for Cas9 to function it also requires a specific protospacer adjacent motif (PAM) that varies depending on the bacterial species of the Cas9 gene. The most commonly used Cas9 nuclease, derived from S. pyogenes, recognizes a PAM sequence of NGG that is found directly downstream of the target sequence in the genomic DNA, on the non-target strand.

### Treatment

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

### Uracil Glycosylase Inhibitor

The term “uracil glycosylase inhibitor” or “UGI,” as used herein, refers to a protein that is capable of inhibiting a uracil-DNA glycosylase base-excision repair enzyme. In some embodiments, a UGI domain comprises a wild-type UGI or a UGI as set forth in SEQ ID NO: 377. In some embodiments, the UGI proteins provided herein include fragments of UGI and proteins homologous to a UGI or a UGI fragment. For example, in some embodiments, a UGI domain comprises a fragment of the amino acid sequence set forth in SEQ ID NO: 377. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 377. In some embodiments, a UGI comprises an amino acid sequence homologous to the amino acid sequence set forth in SEQ ID NO: 377, or an amino acid sequence homologous to a fragment of the amino acid sequence set forth in SEQ ID NO: 377. In some embodiments, proteins comprising UGI or fragments of UGI or homologs of UGI or UGI fragments are referred to as “UGI variants.” A UGI variant shares homology to UGI, or a fragment thereof. For example, a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 377. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 377. In some embodiments, the UGI comprises the following amino acid sequence: MTNLSDIIEKETGKQLVIQESILMLPEEVEEVIGNKPESDILVHTAYDESTDENVMLLTSDAPE YKPWALVIQDSNGENKIKML (SEQ ID NO: 377) (P147391UNGI_BPPB2 Uracil-DNA glycosylase inhibitor), or the same sequence but without the N-terminal methionine.

Other UGI proteins may include those described in Example 6, as follows:

| UGI | Sequence | SEQ ID NO: |
|---|---|---|
| Canonical UGI | TNLSDIIEKETGKQLVIQESILMLPEEVEEVIGNKPESDIVHTAYDES | 378 |
| | TDENVMLLTSDAPEYKPWALVIQDSNGENKIKML | |
| UGI2 | MTLELQLKHYITNLFNLPKDEKWHCESIEEIADDILPDQYVRLGALS | 379 |
| | NKILQTYTYYSDTLHESNIYPFILYYQKQLIAIGYIDENHDMDFLYLH | |
| | NTIMPLLDQRYLLTGGQ | |
| UGI3 | MNKNFDEVKADLRTVTGKKIEFKERLKNILRVQMNQLGFEDSYMIQ | 380 |
| | VQVSSDQEEWVECHENMSLSDFEVMYGNISGEIKRMTVVKYEEANI | |
| | EKLVELKFEYEYAKAHQEYIRAYTKLMSNTLYGRKPSL | |
| UGI5 | MNEEKMHYRDAIKEVELTMMSLDSHFRTHKEFTDSYLLVLILEDVV | 381 |
| | GETRVEVSEGLTFDEASYIIGGTSDNILNMHMINYCEKNREEIYKWL | |
| | KVSRVNTFKSNYAKMLLNTAYGKDLLKGVVK | |
| UGI7 | MNNHFMSIGRNCSKCNNVRLNEDFSKSEEICNECFDKEERFVDSYTL | 382 |
| | IYITEDETGKRFEAILENQTIEETEIIYGNIIDKIIVWNVILTM | |
| UGI12 | DGNEHWEVHPGLSLSDFEVVYGNNPHQIVKLRLDKEVGGSGGSMV | 383 |
| | QNDFIDSYTLCWLLRDDSGGGGSMVQNDFIDSYTLCWLLRDDDGN | |
| | EHWEVHPGLSLSDFEVVYGNNPHQIVKLRLDKEV | |

### Variant

In various embodiments, the evolved DddA-containing base editors or the polypeptides that comprise the evolved DddA-containing base editors (e.g., the pDNAbps and DddA) may be engineered as variants.

As used herein, the term “variant” refers to a protein having characteristics that deviate from what occurs in nature that retains at least one functional i.e. binding, interaction, or enzymatic ability and/or therapeutic property thereof. A “variant” is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type protein. For instance, a variant of Cas9 may comprise a Cas9 that has one or more changes in amino acid residues as compared to a wild type Cas9 amino acid sequence. As another example, a variant of a deaminase may comprise a deaminase that has one or more changes in amino acid residues as compared to a wild type deaminase amino acid sequence, e.g. following ancestral sequence reconstruction of the deaminase. These changes include chemical modifications, including substitutions of different amino acid residues truncations, covalent additions (e.g. of a tag), and any other mutations. The term also encompasses circular permutants, mutants, truncations, or domains of a reference sequence, and which display the same or substantially the same functional activity or activities as the reference sequence. This term also embraces fragments of a wild type protein.

The level or degree of which the property is retained may be reduced relative to the wild type protein but is typically the same or similar in kind. Generally, variants are overall very similar, and in many regions, identical to the amino acid sequence of the protein described herein. A skilled artisan will appreciate how to make and use variants that maintain all, or at least some, of a functional ability or property.

The variant proteins may comprise, or alternatively consist of, an amino acid sequence which is at least 80%, 85%, 90%, 95%, 96%, 97%, 98%, 99%, or 100%, identical to, for example, the amino acid sequence of a wild-type protein, or any protein provided herein (e.g. DddA).

By a polypeptide having an amino acid sequence at least, for example, 95% “identical” to a query amino acid sequence, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98%, or 99% identical to, for instance, the amino acid sequence of a protein such as a DddA protein, can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

### Vector

The term “vector,” as used herein, refers to a nucleic acid that can be modified to encode a gene of interest and that is able to enter into a host cell, mutate and replicate within the host cell, and then transfer a replicated form of the vector into another host cell. Exemplary suitable vectors include viral vectors, such as retroviral vectors or bacteriophages and filamentous phage, and conjugative plasmids. Additional suitable vectors will be apparent to those of skill in the art based on the instant disclosure.

### Wild Type

As used herein the term “wild type” is a term of the art understood by skilled persons and means the typical form of an organism, strain, gene or characteristic as it occurs in nature as distinguished from mutant or variant forms.

These and other exemplary embodiments are described in more detail in the Detailed Description, Examples, and claims. The invention is not intended to be limited in any manner by the above exemplary embodiments.

### DETAILED DESCRIPTION OF CERTAIN EMBODIMENTS

Each mammalian cell contains hundreds to thousands of copies of a circular mtDNA¹⁰. Homoplasmy refers to a state in which all mtDNA molecules are identical, while heteroplasmy refers to a state in which a cell contains a mixture of wild-type and mutant mtDNA. Current approaches to engineer mtDNA rely on DNA-binding proteins such as transcription activator-like effectors nucleases (mitoTALENs)¹¹⁻¹⁷ and zinc finger nucleases (mitoZFNs)¹⁸⁻²⁰ fused to mitochondrial targeting sequences to induce double-strand breaks (DSBs). Such proteins do not rely on nucleic acid programmability (e.g., such as with Cas9 domains). Linearized mtDNA is rapidly degraded,²¹⁻²³ resulting in heteroplasmic shifts to favor uncut mtDNA genomes. As a candidate therapy however, this approach cannot be applied to homoplasmic mtDNA mutations²⁴ since destroying all mtDNA copies is presumed to be harmful.^{22,25} In addition, using DSBs to eliminate heteroplasmic mtDNA mutations, which tend to be functionally recessive,²⁶ implicitly requires the edited cell to restore its wild-type mtDNA copy number. During this transient period of mtDNA repopulation, the loss of mtDNA copies could result in cellular toxicity.

The present disclosure is further to the inventors' discovery of a double-stranded DNA cytidine deaminase, referred to herein as “DddA,” and to its application in base editing of double-stranded nucleic acid molecules, and in particular, the editing of mitochondrial DNA, as described in Mok et al., “A bacterial cytidine deaminase toxin enables CRISPR-free mitochondrial base editing,” Nature, 2020; 583(7817): 631-637 (“Mok et al., 2020”), the entire contents of which are incorporated herein by reference. As depicted in FIG. 1A, the full-length naturally occurring DddA protein is toxic to cells. Without being bound by theory, this cellular toxicity may relate to the fact that the substrate of DddA is any double stranded DNA, including the chromosomal DNA. Thus, as described in Mok et al., the inventors found that the protein could be engineered into split DddA halves that are non-toxic to the cell and inactive on their own until brought together on a target DNA by adjacently bound programmable DNA-binding proteins (e.g., mitoTALE proteins, zinc finger proteins, or Cas9/sgRNA complexes) which bind to the DNA on either side of a site of deamination. The inventors proposed split sites within amino acid loop regions as identified by the crystal structure of DddA. They found that fusions of the split-DddA halves had the ability to deaminate double stranded DNA as a substrate when brought together at a site of deamination by a pair of programmable DNA binding proteins binding to different sites at a deamination site (or edit site).

As now disclosed herein, the inventors have used continuous evolution methods, e.g., phage-assisted non-continuous evolution (PANCE) and phage-assisted continuous evolution (PACE), for example, as illustrated in FIG. 2, to evolve a starting point DddA protein or fragment thereof to form an evolved variant DddA or evolved fragment of DddA having one or more improved characteristics, including increase deaminase activity and/or expanded sequence contexts in which deamination may occur.

The present disclosure provides methods for making such DddA variants, methods of making base editors comprising said variants, base editors comprising fusion proteins of an evolved variant DddA and a programmable DNA binding protein (e.g., a mitoTALE, zinc finger, or napDNAbp), the variant DddA proteins themselves, DNA vectors encoding said base editors, methods for delivery said based editors to cells, and methods for using said base editors to edit a target double stranded DNA molecule, including a mitochondrial genome.

FIG. 1A is a schematic representation of a naturally occurring DddA, an interbacterial toxin discovered by the inventors which was found to catalyze deamination of cytidines within double-stranded DNA as a substrate. The inventors are believed to be the first to identify such a deaminase. However, in its naturally occurring form, the inventors discovered that DddA is toxic to cells. The inventors have conceived of the idea of using the DddA in the context of base editing to deaminate a nucleobase at a target edit site.

In the context of base editing, all previously described cytidine deaminases utilize single-stranded DNA as a substrate (e.g., the R-loop region of a Cas9-gRNA/dsDNA complex). Base editing in the context of mitochondrial DNA has not heretofore been possible due to the challenges of introducing and/or expressing the gRNA needed for a Cas9-based system into mitochondria. The inventors have recognized for the first time that the catalytic properties of DddA can be leveraged to conduct base editing directly on a double strand DNA substrate by separating the DddA into inactive portions, which when co-localized within a cell will become reconstituted as an active DddA. This avoids or at least minimizes the toxicity associated with delivering and/or expressing a fully active DddA in a cell.

For example, a DddA may be divided into two fragments at a “split site,” i.e., a peptide bond between two adjacent residues in the primary structure or sequence of a DddA. The split site may be positioned anywhere along the length of the DddA amino acid sequence, so long as the resulting fragments do not on their own possess a toxic property (which could be a complete or partial deaminase activity). In certain embodiments, the split site is located in a loop region of the DddA protein. In the embodiment shown in FIG. 1A, the arrows depict five possible split sites approximately equally spaced along the length of the DddA protein. The depicted embodiment further shows that the DddA was divided into two fragments at a split site located approximately in the middle of the DddA amino acid sequence. The DddA fragment lying to the left of the split site may be referred to as the “N-terminal DddA half” and the DddA fragment lying to the right of the split site may be referred to as the “C-terminal DddA half.” FIG. 1A identifies these fragments as “DddA half^{A}” and DddA half^{B}”, respectively. Depending on the location of the split site, the N-terminal DddA half and the C-terminal DddA half could be the same size, approximately the same size, or very different sizes.

Accordingly, this disclosure provides compositions, kits, and methods of modifying double-stranded DNA (e.g., mitochondrial DNA or “mtDNA”) using genome editing strategies that comprise the use of a programmable DNA binding protein (“pDNAbp”) (e.g., a mitoTALE, mitoZFP, or a CRISPR/Cas9) and a double-stranded DNA deaminase (“DddA”) to precisely install nucleotide changes and/or correct pathogenic mutations in double-stranded DNA (e.g., mtDNA), rather than destroying the DNA (e.g., mtDNA) with double-strand breaks (DSBs). The present disclosure provides pDNAbp polypeptides, DddA polypeptides, fusion proteins comprising pDNAbp polypeptides and DddA polypeptides, nucleic acid molecules encoding the pDNAbp polypeptides, DddA polypeptides, and fusion proteins described herein, expression vectors comprising the nucleic acid molecules described herein, cells comprising the nucleic acid molecules, expression vectors, pDNAbp polypeptides, DddA polypeptides, and/or fusion proteins described herein, pharmaceutical compositions comprising the polypeptides, fusion proteins, nucleic acid molecules, vectors, or cells described herein, and kits comprising the polypeptides, fusion proteins, nucleic acid molecules, vectors, or cells described herein for modifying double-stranded DNA (e.g., mtDNA) by base editing.

Mitochondrial diseases (e.g., MELAS/Leigh syndrome and Leber's hereditary optic neuropathy) are diseases often resulting from errors or mutations in the mitochondrial DNA (mtDNA). In many cases, the mutated mtDNA co-exists with the wild-type mtDNA (mtDNA heteroplasmy). In such instances, residual wild type mtDNA can partially compensate for the mutation before biochemical and clinical manifestations occur. Multiple approaches to reduce the levels of mutant mtDNA have been tried. None of these approaches, however, have been successful in treating or correcting these abnormalities. The present disclosure, including the disclosed DddA/pDNAbp fusion proteins, nucleic acid molecules and vectors encoding same can be used to treat one or more mitochondrial diseases, which can include, but are not limited to: Alper's Disease, Autosomal Dominant Optic Atrophy (ADOA), Barth Syndrome, Carnitine Deficiency, Chronic Progressive External Ophthalmoplegia (CPEO), Co-Enzyme Q10 Deficiency, Creatine Deficiency Syndrome, Fatty Acid Oxidation Disorders, Friedreich's Ataxia, Kearns-Sayre Syndrome (KSS), Lactic Acidosis, Leber Hereditary Optic Neuropathy (LHON), Leigh Syndrome, MELAS, Mitochondrial Myopathy, Multiple Mitochondrial Dysfunction Syndrome, Primary Mitochondrial Myopathy, and TK2d, among others.

The present disclosure addresses many of the shortcomings of the existing technologies with a new precision mtDNA editing fusion protein and technique. The proposed technology permits the editing (e.g., deamination) of single, or multiple, nucleotides in the mtDNA allowing for the correction or modification of the nucleotide, and by extension the codon in which it is contained. In various embodiment, however, the present disclosure is not limited to editing mtDNA, but may also be used to target the editing of any double-stranded DNA in the cell, including the genomic DNA in the nucleus.

### I. Evolved DddA Variants

In various embodiments, the Evolved DddA-containing base editors or the polypeptides that comprise the Evolved DddA-containing base editors (e.g., the pDNAbps and DddA) may be engineered to include any variant of any DddA, or an inactive fragment thereof. In certain embodiments, the DddA variant may be obtained through a continuous evolution process, such as PACE. The term “phage-assisted continuous evolution (PACE),” as used herein, refers to continuous evolution that employs phage as viral vectors and is described in Thuronyi, B. W. et al. Nat Biotechnol 37, 1070-1079 (2019), the contents of which are incorporated herein by reference in their entirety. The general concept of PACE technology has also been described, for example, in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Application, U.S. Pat. No. 9,023,594, issued May 5, 2015, International PCT Application, PCT/US2015/012022, filed Jan. 20, 2015, published as WO 2015/134121 on Sep. 11, 2015, and International PCT Application, PCT/US2016/027795, filed Apr. 15, 2016, published as WO 2016/168631 on Oct. 20, 2016, the entire contents of each of which are incorporated herein by reference. PACE can be used, for instance, to evolve a deaminase (e.g., a cytidine or adenosine deaminase) which uses single strand DNA as a substrate to obtain a deaminase which is capable of using double-strand DNA as a substrate (e.g., DddA).

In various embodiments involving obtaining a DddA variant by way of one or more mutagenesis methodologies, such as, but not limited to a continuous evolution process (e.g., PACE), the process may begin with a “starter” protein, such as canonical DddA or a fragment of DddA, such as DddAtox, which corresponds to the N-terminal portion of canonical DddA.

In various embodiments, the starter DddA protein from which variants are derived can be the canonical protein, or a fragment there. As reported in Mok et al. 2020, the DddA was discovered in Burkholderia cenocepia and reported in the Protein Data Bank as PDB ID: 6U08, which has the following full-length amino acid sequence (1427 amino acids):

>tr|A0A1V6L4E7|A0A1V6L4E7_9BURK YD repeat (Two copies) OS = Burkholderia
cenocepacia OX = 95486 GN = UE95_03830 PE = 1 SV = 1
(SEQ ID NO: 16)
MYEAARVTDPIDHTSALAGFLVGAVLGIALIAAVAFATFTCGFGVALLAGMMAGIGAQALLSIGESIG
KMFSSQSGNIITGSPDVYVNSLSAAYATLSGVACSKHNPIPLVAQGSTNIFINGRPAARKDDKITCGATI
GDGSHDTFFHGGTQTYLPVDDEVPPWLRTATDWAFTLAGLVGGLGGLLKASGGLSRAVLPCAAKFIG
GYVLGEAFGRYVAGPAINKAIGGLFGNPIDVTTGRKILLAESETDYVIPSPLPVAIKRFYSSGIDYAGTL
GRGWVLPWEIRLHARDGRLWYTDAQGRESGFPMLRAGQAAFSEADQRYLTRTPDGRYILHDLGERY
YDFGQYDPESGRIAWVRRVEDQAGQWYQFERDSRGRVTEILTCGGLRAVLDYETVFGRLGTVTLVH
EDERRLAVTYGYDENGQLASVTDANGAVVRQFAYTNGLMTSHMNALGFTSSYVWSKIEGEPRVVET
HTSEGENWTFEYDVAGRQTRVRHADGRTAHWRFDAQSQIVEYTDLDGAFYRIKYDAVGMPVMLML
PGDRTVMFEYDDAGRIIAETDPLGRTTRTRYDGNSLRPVEVVGPDGGAWRVEYDQQGRVVSNQDSL
GRENRYEYPKALTALPSAHIDALGGRKTLEWNSLGKLVGYTDCSGKTTRTSFDAFGRICSRENALGQR
ITYDVRPTGEPRRVTYPDGSSETFEYDAAGTLVRYIGLGGRVQELLRNARGQLIEAVDPAGRRVQYRY
DVEGRLRELQQDHARYTFTYSAGGRLLTETRPDGILRRFEYGEAGELLGLDIVGAPDPHATGNRSVRT
IRFERDRMGVLKVQRTPTEVTRYQHDKGDRLVKVERVPTPSGIALGIVPDAVEFEYDKGGRLVAEHG
SNGSVIYTLDELDNVVSLGLPHDQTLQMLRYGSGHVHQIRFGDQVVADFERDDLHREVSRTQGRLTQ
RSGYDPLGRKVWQSAGIDPEMLGRGSGQLWRNYGYDAAGDLIETSDSLRGSTRFSYDPAGRLISRAN
PLDRKFEEFAWDAAGNLLDDAQRKSRGYVEGNRLLMWQDLRFEYDPFGNLATKRRGANQTQRFTY
DGQDRLITVHTQDVRGVVETRFAYDPLGRRIAKTDTAFDLRGMKLRAETKRFVWEGLRLVQEVRET
GVSSYVYSPDAPYSPVARADTVMAEALAATVIDSAKRAARIFHFHTDPVGAPQEVTDEAGEVAWAG
QYAAWGKVEATNRGVTAARTDQPLRFAGQYADDSTGLHYNTFRFYDPDVGRFINQDPIGLNGGANV
YHYAPNPVGWVDPWGLAGSYALGPYQISAPQLPAYNGQTVGTFYYVNDAGGLESKVFSSGGPTPYP
NYANAGHVEGQSALFMRDNGISEGLVFHNNPEGTCGFCVNMTETLLPENAKMTVVPPEGAIPVKRGA
TGETKVFTGNSNSPKSPTKGGC.

In various other embodiments, the starter protein can be a DddA fragment. For instance, a starter DddA protein can be a DddA fragment having the following amino acid sequence:

(SEQ ID NO: 25)
GSYALGPYQISAPQLPAYNGQTVGTFYYVNDAGGLESKVFSSGGPTPYP
NYANAGHVEGQSALFMRDNGISEGLVFHNNPEGTCGFCVNMTETLLPEN
AKMTVVPPEGAIPVKRGATGETKVFTGNSNSPKSPTKGGC,

or an amino acid sequence having at least 70%, at least 75%, at least 80%, at least 85%, at least 90%, at least 95%, at least 96%, at least 97%, at least 98%, at least 99%, or 100% sequence identify with DddA of SEQ ID NO: 25, or a fragment thereof.

In other embodiments, the DddA has the following amino acid sequence:

(SEQ ID NO: 26)
XGSSHHHHHHSQDPIGLNGGANVYHYAPNPVGWVDPWGLAGSYALGPYQ
ISAPQLPAYNGQTVGTFYYVNDAGGLESKVFSSGGPTPYPNYANAGHVE
GQSALFXRDNGISEGLVFHNNPEGTCGFCVNXTETLLPENAKXTVVPPE
GAIPVKRGATGETKVFTGNSNSPKSPTKGGC

(which corresponds to the N-terminal portion of canonical DddA of PDB Accession No. 6U08_A of Burkholderia cenocepacia and includes a HisTag sequence), and can include fragments or variants thereof, including amino acid sequences having at least 70%, at least 75%, at least 80%, at least 85%, at least 90%, at least 95%, at least 96%, at least 97%, at least 98%, at least 99%, or 100% sequence identify with SEQ ID NO: 26.

In various other embodiments, the starter DddA protein can be a split DddA can have the following sequences:

### G1333 DddAtox-N¬

GSYALGPYQISAPQLPAYNGQTVGTFYYVNDAGGLESKVFSSGG (SEQ ID NO: 338), and can include fragments or variants thereof, including amino acid sequences having at least 70%, at least 75%, at least 80%, at least 85%, at least 90%, at least 95%, at least 96%, at least 97%, at least 98%, at least 99%, or 100% sequence identify with DddA of SEQ ID NO: 338.

### G1333 DddAtox-C

PTPYPNYANAGHVEGQSALFMRDNGISEGLVFHNNPEGTCGFCVNMTETLLPENAKMTVVP PEGAIPVKRGATGETKVFTGNSNSPKSPTKGGC (SEQ ID NO: 339), and can include fragments or variants thereof, including amino acid sequences having at least 70%, at least 75%, at least 80%, at least 85%, at least 90%, at least 95%, at least 96%, at least 97%, at least 98%, at least 99%, or 100% sequence identify with DddA of SEQ ID NO: 339.

### G1397 DddAtox-N¬

GSYALGPYQISAPQLPAYNGQTVGTFYYVNDAGGLESKVFSSGGPTPYPNYANAGHVEGQS ALFMRDNGISEGLVFHNNPEGTCGFCVNMTETLLPENAKMTVVPPEG (SEQ ID NO: 340), and can include fragments or variants thereof, including amino acid sequences having at least 70%, at least 75%, at least 80%, at least 85%, at least 90%, at least 95%, at least 96%, at least 97%, at least 98%, at least 99%, or 100% sequence identify with DddA of SEQ ID NO: 340.

### G1397 DddAtox-C

AIPVKRGATGETKVFTGNSNSPKSPTKGGC (SEQ ID NO: 341), and can include fragments or variants thereof, including amino acid sequences having at least 70%, at least 75%, at least 80%, at least 85%, at least 90%, at least 95%, at least 96%, at least 97%, at least 98%, at least 99%, or 100% sequence identify with DddA of SEQ ID NO: 341.

### Split DddA (DddA-G1397N)

GSYALGPYQISAPQLPAYNGQTVGTFYYVNDAGGLESKVFSSGGPTPYPNYANAGHVEGQS ALFMRDNGISEGLVFHNNPEGTCGFCVNMTETLLPENAKMTVVPPEG (SEQ ID NO: 340), and can include fragments or variants thereof, including amino acid sequences having at least 70%, at least 75%, at least 80%, at least 85%, at least 90%, at least 95%, at least 96%, at least 97%, at least 98%, at least 99%, or 100% sequence identify with DddA of SEQ ID NO: 340.

### Split DddA (DddA-G1397C)

AIPVKRGATGETKVFTGNSNSPKSPTKGGC (SEQ ID NO: 341).

The disclosure also contemplates the use of any variant of DddAtox, or proteins comprising an amino acid sequence having at least 70%, at least 75%, at least 80%, at least 85%, at least 90%, at least 95%, at least 96%, at least 97%, at least 98%, at least 99%, or 100% sequence identify with DddA-G1397C, or a biologically active fragment of DddA-G1397C.

As shown in FIG. 1A, the present inventors have recognized that the whole, intact DddA is toxic to cells. Thus, in order to utilize the DddA in the context of the Evolved DddA-containing base editors described herein, the DddA must be delivered in an inactive form. One of ordinary skill in the art will appreciate that various methods, techniques, and modification known in the art can be adapted for reversibly inactivating DddA such that the enzyme may be delivered to a cell in an inactive state, but then become activated inside the cell (or the mitochondria) under one or more conditions, or in the presence of one or more inducing agents, in order to conduct the desired deamination.

In preferred embodiments, as depicted in FIGS. 1A-1F, the DddA may be split into inactive fragments which can be separately delivered to a target deamination site on separate fusion constructs that target each fragment of the DddA to sites positioned on either side of a target edit site.

In some embodiments, the DddA comprises a first portion and a second portion. In some embodiments, the first portion and the second portion together comprise a full length DddA. In some embodiments, the first and second portion comprise less than the full length DddA portion. In some embodiments, the first and second portion independently do not have any, or have minimal, native DddA activity (e.g., deamination activity). In some embodiment, the first and second portion can re-assemble (i.e., dimerize) into a DddA protein with, at least partial, native DddA activity (e.g., deamination activity).

In some embodiments, the first and second portion of the DddA are formed by truncating (i.e., dividing or splitting the DddA protein) at specified amino acid residues. In some embodiments, the first portion of a DddA comprises a full-length DddA truncated at its N-terminus. In some embodiments, the second portion of a DddA comprises a full-length DddA truncated at its C-terminus. In some embodiments, additional truncations are performed to either the full-length DddA or to the first or second portions of the DddA. In some embodiments, the first and second portions of a DddA may comprise additional truncations, but which the first and second portion can dimerize or re-assemble, to restore, at least partially, native DddA activity (e.g., deamination). In some embodiments, the first and second portions comprise full-length DddA truncated at, or around, a residue in DddA selected from the group comprising: 62, 71, 73, 84, 94, 108, 110, 122, 135, 138, 148, and 155. In some embodiments, the truncation of DddA occurs at residue 148.

In certain embodiments, the DddA can be separated into two fragments by dividing the DddA at a split site. A “split site” refers to a position between two adjacent amino acids (in a wildtype DddA amino acid sequence) that marks a point of division of a DddA. In certain embodiments, the DddA can have a least one split site, such that once divided at that split site, the DddA forms an N-terminal fragment and a C-terminal fragment. The N-terminal and C-terminal fragments can be the same or difference sizes (or lengths), wherein the size and/or polypeptide length depends on the location or position of the split site. As used herein, reference to a “fragment” of DddA (or any other polypeptide) can be referred equivalently as a “portion.” Thus, a DddA which is divided at a split site can form an N-terminal portion and a C-terminal portion. Preferably, the N-terminal fragment (or portion) and the C-terminal fragment (or portion) or DddA do not have a deaminase activity.

In various embodiments, a DddA may be split into two or more inactive fragments by directly cleaving the DddA at one or more split sites. Direct cleaving can be carried out by a protease (e.g., trypsin) or other enzyme or chemical reagent. In certain embodiments, such chemical cleavage reactions can be designed to be site-selective (e.g., Elashal and Raj, “Site-selective chemical cleavage of peptide bonds,” Chemical Communications, 2016, Vol. 52, pages 6304-6307, the contents of which are incorporated herein by reference.) In other embodiments, chemical cleavage reactions can be designed to be non-selective and/or occur in a random fashion.

In other embodiments, the two or more inactive DddA fragments can be engineered as separately expressed polypeptides. For instance, for a DddA having one split site, the N-terminal DddA fragment could be engineered from a first nucleotide sequence that encodes the N-terminal DddA fragment (which extends from the N-terminus of the DddA up to and including the residue on the amino-terminal side of the split site). In such an example, the C-terminal DddA fragment could be engineered from a second nucleotide sequence that encodes the C-terminal DddA fragment (which extends from the carboxy-terminus of the split site up to including the natural C-terminus of the DddA protein). The first and second nucleotide sequences could be on the same or different nucleotide molecules (e.g., the same or different expression vectors).

In various embodiments, that N-terminal portion of the DddA may be referred to as “DddA-N half” and the C-terminal portion of the DddA may be referred to as the “DddA-C half.” Reference to the term “half” does not connote the requirement that the DddA-N and DddA-C portions are identically half of the size and/or sequence length of a complete DddA, or that the split site is required to be at the mid point of the complete DddA polypeptide. To the contrary, and as noted above, the split site can be between any pair of residues in the DddA polypeptide, thereby giving rise to half portions which are unequal in size and/or sequence length. In certain embodiments, the split site is within a loop region of the DddA.

Accordingly, in one aspect, the disclosure relates to a pair of fusion proteins useful for making modifications to the sequence of mitochondrial DNA (e.g., mtDNA). The pair of fusion proteins, in some embodiments, can comprise a first fusion protein comprising a first pDNAbp (e.g., a mitoTALE, mitoZFP, or a CRISPR/Cas9) and a first portion or fragment of a DddA, and a second fusion protein comprising a second pDNAbp (e.g., mitoTALE, mitoZFP, or a CRISPR/Cas9) and a second portion or fragment of a DddA, such that the first and the second portions of the DddA reconstitute a DddA upon co-localization in a cell and/or mitochondria. In certain embodiments, that first portion of the DddA is an N-terminal fragment of a DddA and the second portion of the DddA is C-terminal fragment of a DddA. In other embodiments, the first portion of the DddA is a C-terminal fragment of a DddA and the second portion of the DddA is an N-terminal fragment of a DddA. In this aspect, the structure of the pair of fusion proteins can be, for example:
[pDNAbp]-[DddA half^{A}] and [pDNAbp]-[DddA half^{B}];
[DddA-half^{A}]-[pDNAbp] and [DddA-half^{B}]-[pDNAbp];
[pDNAbp]-[DddA half^{A}] and [DddA-half^{B}]-[pDNAbp]; or
[DddA-half^{A}]-[pDNAbp] and [pDNAbp]-[DddA half^{B}], wherein “A” or “B” can be the N-terminal or C-terminal half of DddA.

In another aspect, the disclosure relates to a pair of fusion proteins useful for making modifications to the sequence of mitochondrial DNA (e.g., mtDNA). The pair of fusion proteins can comprise a first fusion protein comprising a first mitoTALE and a first portion or fragment of a DddA, and a second fusion protein comprising a second mitoTALE and a second portion or fragment of a DddA, such that the first and the second portions of the DddA, upon co-localization in a cell and/or mitochondria, are reconstituted an active DddA. In certain embodiments, that first portion of the DddA is an N-terminal fragment of a DddA and the second portion of the DddA is C-terminal fragment of a DddA. In other embodiments, the first portion of the DddA is a C-terminal fragment of a DddA and the second portion of the DddA is an N-terminal fragment of a DddA. In this aspect, the structure of the pair of fusion proteins can be, for example:
[mitoTALE]-[DddA half^{A}] and [mitoTALE]-[DddA half^{B}];
[DddA-half^{A}]-[pDNAbp] and [DddA-half^{B}]-[mitoTALE];
[mitoTALE]-[DddA half^{A}] and [DddA-half^{B}]-[mitoTALE]; or
[DddA-half^{A}]-[mitoTALE] and [mitoTALE]-[DddA half^{B}], wherein “A” or “B” can be the N-terminal or C-terminal half of DddA.

In yet another aspect, the disclosure relates to a pair of fusion proteins useful for making modifications to the sequence of mitochondrial DNA (e.g., mtDNA). The pair of fusion proteins can comprise a first fusion protein comprising a first mitoZFP and a first portion or fragment of a DddA, and a second fusion protein comprising a second mitoZFP and a second portion or fragment of a DddA, such that the first and the second portions of the DddA, upon co-localization in a cell and/or mitochondria, are reconstituted an active DddA. In certain embodiments, that first portion of the DddA is an N-terminal fragment of a DddA and the second portion of the DddA is C-terminal fragment of a DddA. In other embodiments, the first portion of the DddA is a C-terminal fragment of a DddA and the second portion of the DddA is an N-terminal fragment of a DddA. In this aspect, the structure of the pair of fusion proteins can be, for example:
[mitoZFP]-[DddA half^{A}] and [mitoZFP]-[DddA half^{B}];
[DddA-half^{A}]-[pDNAbp] and [DddA-half^{B}]-[mitoZFP];
[mitoZFP]-[DddA half^{A}] and [DddA-half^{B}]-[mitoZFP]; or
[DddA-half^{A}]-[mitoZFP] and [mitoZFP]-[DddA half^{B}], wherein “A” or “B” can be the N-terminal or C-terminal half of DddA.

In yet another aspect, the disclosure relates to a pair of fusion proteins useful for making modifications to the sequence of mitochondrial DNA (e.g., mtDNA). The pair of fusion proteins can comprise a first fusion protein comprising a first Cas9 and a first portion or fragment of a DddA, and a second fusion protein comprising a second Cas9 and a second portion or fragment of a DddA, such that the first and the second portions of the DddA, upon co-localization in a cell and/or mitochondria, are reconstituted an active DddA. In certain embodiments, that first portion of the DddA is an N-terminal fragment of a DddA (i.e., “DddA half^{A}” as shown in FIGS. 1A-1E) and the second portion of the DddA is C-terminal fragment of a DddA (i.e., “DddA half^{B}” as shown in FIGS. 1A-1E). In other embodiments, the first portion of the DddA is an C-terminal fragment of a DddA and the second portion of the DddA is an N-terminal fragment of a DddA. In this aspect, the structure of the pair of fusion proteins can be, for example:
[Cas9]-[DddA half^{A}] and [Cas9]-[DddA half^{B}];
[DddA-half^{A}]-[Cas9] and [DddA-half^{B}]-[Cas9];
[Cas9]-[DddA half^{A}] and [DddA-half^{B}]-[Cas9]; or
[DddA-half^{A}]-[Cas9] and [Cas9]-[DddA half^{B}], wherein “A” or “B” can be the N-terminal or C-terminal half of DddA.

In each instance above of “]-[” can be in reference to a linker sequence.

In some embodiments, a first fusion protein comprises, a first mitochondrial transcription activator-like effector (mitoTALE) domain and a first portion of a DNA deaminase effector (DddA).

In some embodiments, the first portion of the DddA comprises an N-terminal truncated DddA. In some embodiments, the first mitoTALE is configured to bind a first nucleic acid sequence proximal to a target nucleotide. In some embodiments, the first portion of a DddA is linked to the remainder of the first fusion protein by the C-terminus of the first portion of a DddA.

In one aspect, the present disclosure provides mitochondrial DNA editor fusion proteins for use in editing mitochondrial DNA. As used herein, these mitochondrial DNA editor fusion proteins may be referred to as “mtDNA editors” or “mtDNA editing systems.”

In various embodiments, the mtDNA editors described herein comprise (1) a programmable DNA binding protein (“pDNAbp”) (e.g., a mitoTALE domain, mitoZFP domain, or a CRISPR/Cas9 domain) and a double-stranded DNA deaminase domain, which is capable of carrying out a deamination of a nucleobase at a target site associated with the binding site of the programmable DNA binding protein (pDNAbp).

In some embodiments, the double-stranded DNA deaminase is split into two inactive half portions, with each half portion being fused to a programmable DNA binding protein that binds to a nucleotide sequence either upstream or downstream of a target edit site, and wherein once in the mitochondria, the two half portions (i.e., the N-terminal half and the C-terminal half) reassociate at the target edit site by the co-localization of the programmable DNA binding proteins to binding sites upstream and downstream of the target edit site to be acted on by the DNA deaminase. The reassociation of the two half portions of the double-stranded DNA deaminase restores the deaminase activity at the target edit site. In other embodiments, the double-stranded DNA deaminase can initially be set in an inactive state which can be induced when in the mitochondria. The double-stranded DNA deaminase is preferably delivered initially in an inactive form in order to avoid toxicity inherent with the protein. Any means to regulate the toxic properties of the double-stranded DNA deaminase until such time as the activity is desired to be activated (e.g., in the mitochondria) is contemplated.

In various embodiments, the following exemplary DddA enzymes, or variants thereof, can be used with the Evolved DddA-containing base editors described herein, or a sequence (amino acid or nucleotide as the case may be) having at least 70%, 75%, 80%, 85%, 90%, 95%, or 99% sequence identity with an one of the following DddA sequences:

| DddA Description | DddA amino acid and/or nucleotide sequence |
|---|---|
| DddA homolog in | >ATF83755.1 hypothetical protein CO712_00910 [Burkholderia gladioli |
| Burkholderia | pv. gladioli] |
| gladioli | MYEAARVTDPIEHTSALAGFLVGAVLGIALIAAVAFATFTCGFGVALLAGMAAGIGAQ |
| PROTEIN | VLLSLGESIGKMFSSQSGAITLGSPNVYVNGKQAAYATLSSVTCSKHNPTPLVAQGST |
| | NIFINGKPAARKDDKITCGAAISDGSHDTYFHGGIQTCLPIDDEVPPWLRTATDWAFAL |
| | AGLVGGLGGLLKEAGGLSHAVMPCAAKFIGGYVLGEAASRYVIGPAINSAIGGMFGN |
| | PVDVTTGRKILPAESETDYVVPSPMPVAIRRFYSSDLDYVGTLGRGWVLPWELRLHAR |
| | DGRLWYTDAQGRESGFPILKPGQAAFSEADQRYLTCTPDGRYILHDVGETYYDFGRY |
| | EPGSGRIGWVRRIEDQAGQWCQFERDSRGRVREIQTCGGLLAVLDYEPEHERLAEVSL |
| | VSGDQRRLVVAYGYDENGQMASVTDANGAVVRRFTYADGRMTSHSNALGFTSGYT |
| | WKVIDGTPRVVATHTSEGEAWAFEYDIEGRRTHVRHADGRHAQWRYDAQFQIVEYL |
| | DFDGRRYGLKYNAAGMPVMLTLPGERTVMFEYDDAGRIVAETDPLGRTTKTRYDGN |
| | SMRPVEIILPDGSAWHAEYDRQGRLLVTRDPLDRENRYEYPEALSALPVAHVDALGG |
| | RKTFEWNRLGELVAYTDCSGKTTRNFFDAFGLPLARENALGHRVSFDLRPTGETRRVT |
| | YPDGSSESYEYDAAGLMIRHIGLGGRMQTLQRNARGQLVEAVDPAGRRTRYHYDAE |
| | GRLRELQQAHARYAFAYSAGGRLVSETRPDGVLRRFEYGEAGDLAALEIVGTADDCA |
| | PNDRPVRAIRFERDRMGNLCVQHTPTEVTRYERDAGGRLLEVASVPTAAGLALGIAPD |
| | TLTFEYDKAGRLSAEHGANGSVQYTLDALDNVLKLALPHEQTLQMLRYGSGHVHQIR |
| | HGDQVVSDFERDDLHRELTRTQGPLTERTAYDLLGRKIWQSAGFQPDALARGQGQL |
| | WRNYGYDAAGELVESHDSLRGSTQFSYDPAGYLTQRVNTADRQLESFAWDAAGNLL |
| | DDAQRSSRGYVEGNRLRMWQNLRFDYDAFGNLATKLRGANQRQQFTYDGQDRLVA |
| | VRTQGARGVVETRFAYDPLGRRIAKTDRTLDVRGVTLREETKRFVWEGLRLAQEVRD |
| | TGVSSYVYSPDAPYMPAARVDAVKAEALANAAIDKARQATRIYHFHTDVSGAPQEAT |
| | NEAGDIVWAGQYSAWGKVAPNQHAPARIDQPLRYAGQYADDSTELHYNTFRFYDPD |
| | VGRFINQDPIGLMGGLNLYQYAPNSIAWTDWWGLAGSYTLGSYQISAPQLPAYNGQT |
| | VGTFYYVNDAGGLESRTFSSGGPTPYPNYANAGHVEGQSALFMRDNGISDGLVFHNN |
| | PEGTCGFCVNMTETLLPENSKLTVVPPEGSIPVKRGATGETRTFTGNSKSPKSPVKGGC |
| | [SEQ ID NO: 130] |
| DddA homolog in | >CO712_00910 NZ_CP023522.1:185368-189645 Burkholderia gladioli pv. |
| Burkholderia | gladioli strain FDAARGOS_389 chromosome 1, complete sequence |
| gladioli | GTGTACGAAGCGGCCCGCGTCACGGATCCGATCGAGCACACCAGCGCGCTGGCCG |
| DNA | GCTTCCTGGTGGGCGCCGTGCTCGGTATCGCCCTGATTGCTGCCGTGGCGTTCGCC |
| | ACGTTCACCTGCGGCTTCGGCGTGGCACTGCTGGCCGGCATGGCGGCCGGCATCG |
| | GCGCGCAGGTGCTGTTGTCGTTAGGGGAATCGATCGGGAAGATGTTCAGTTCGCA |
| | ATCCGGCGCGATCACGCTCGGCTCGCCGAACGTCTACGTGAACGGCAAGCAGGCC |
| | GCCTACGCCACGCTCAGCAGCGTGACGTGCAGCAAGCACAACCCGACGCCGCTCG |
| | TCGCGCAGGGCTCCACCAACATCTTCATCAACGGCAAGCCGGCCGCGCGCAAGGA |
| | CGACAAGATCACCTGCGGCGCGGCCATCTCGGACGGCTCGCACGACACCTACTTC |
| | CACGGAGGCATCCAGACCTGCCTGCCGATCGACGACGAAGTGCCGCCGTGGCTGC |
| | GCACCGCCACCGACTGGGCGTTCGCGCTGGCCGGGCTGGTGGGCGGGCTCGGCGG |
| | CCTACTCAAGGAAGCGGGCGGGCTGTCGCACGCGGTGATGCCGTGCGCGGCGAAG |
| | TTCATCGGCGGCTACGTGCTCGGCGAGGCGGCGAGCCGCTACGTGATCGGCCCGG |
| | CCATCAACAGCGCGATCGGCGGGATGTTCGGCAACCCGGTAGACGTCACCACTGG |
| | GCGCAAGATCCTCCCTGCCGAATCGGAAACCGATTACGTCGTGCCCAGCCCGATG |
| | CCGGTGGCGATCCGGCGCTTCTATTCGAGCGACCTCGATTACGTCGGCACGCTTGG |
| | GCGCGGCTGGGTGCTGCCGTGGGAGCTGCGCCTGCACGCGCGTGACGGTCGGCTC |
| | TGGTACACCGACGCGCAGGGGCGCGAGAGCGGCTTCCCGATCCTGAAACCGGGCC |
| | AGGCCGCGTTCAGCGAGGCCGATCAGCGCTATCTGACCTGCACGCCGGATGGCCG |
| | CTACATCCTCCACGACGTCGGCGAAACCTATTACGACTTCGGCCGCTACGAGCCGG |
| | GCTCGGGCCGCATCGGCTGGGTGCGCCGGATCGAGGATCAGGCCGGCCAGTGGTG |
| | CCAGTTCGAGCGCGACAGCCGTGGCCGCGTGCGTGAAATCCAGACCTGCGGCGGC |
| | TTGCTGGCCGTGCTCGATTACGAGCCGGAGCACGAGCGGCTCGCCGAGGTGTCGC |
| | TCGTCAGCGGCGATCAGCGCCGCCTCGTCGTGGCCTACGGCTACGACGAAAACGG |
| | CCAGATGGCCTCCGTGACCGACGCGAACGGCGCGGTGGTGCGCCGCTTCACCTAT |
| | GCCGACGGGCGCATGACGAGCCATTCGAACGCGCTCGGTTTCACGTCGGGCTATA |
| | CGTGGAAGGTCATCGACGGCACGCCGCGAGTGGTCGCCACCCACACCAGCGAGGG |
| | CGAGGCCTGGGCGTTCGAGTACGACATCGAAGGCCGCCGCACCCATGTGCGGCAT |
| | GCCGACGGCCGCCACGCGCAATGGCGCTACGACGCGCAATTCCAGATCGTCGAGT |
| | ACCTCGATTTCGACGGCCGTCGCTACGGGCTCAAGTACAACGCTGCCGGCATGCCC |
| | GTGATGCTGACGCTGCCCGGCGAACGAACCGTGATGTTCGAGTACGACGACGCCG |
| | GCCGCATCGTCGCCGAAACCGATCCCCTCGGCCGCACCACGAAAACGCGCTACGA |
| | CGGCAACAGCATGCGGCCCGTCGAGATCATCTTGCCCGACGGCAGCGCCTGGCAC |
| | GCCGAATACGACCGGCAGGGCCGGCTGCTCGTCACCCGTGATCCGCTCGACCGGG |
| | AGAATCGCTACGAATATCCGGAGGCACTGAGCGCGCTCCCGGTGGCGCATGTCGA |
| | TGCGCTGGGCGGGCGCAAGACGTTCGAGTGGAACCGGCTCGGCGAGCTGGTGGCC |
| | TACACCGATTGCTCGGGCAAGACCACGCGCAATTTTTTCGATGCATTCGGCCTGCC |
| | GCTCGCGCGCGAGAACGCGCTCGGGCACCGCGTGTCGTTCGATCTGCGCCCGACC |
| | GGCGAGACGCGCCGCGTCACCTATCCCGACGGCAGTTCCGAAAGCTACGAATACG |
| | ACGCCGCCGGGCTGATGATCCGGCACATCGGGCTGGGCGGCCGGATGCAGACGTT |
| | GCAGCGCAATGCGCGCGGGCAACTCGTCGAGGCGGTCGATCCGGCCGGGCGGCGA |
| | ACCCGCTACCACTACGACGCCGAAGGGCGGCTGCGCGAGCTGCAACAGGCCCACG |
| | CGCGCTACGCATTCGCGTACAGCGCAGGCGGGCGGCTTGTCAGCGAAACGCGGCC |
| | CGACGGCGTGCTGCGCCGCTTCGAATACGGCGAGGCCGGCGATCTGGCGGCGCTC |
| | GAGATCGTCGGAACGGCCGATGATTGCGCTCCAAACGATCGCCCGGTTCGCGCGA |
| | TCCGCTTCGAGCGCGACCGGATGGGTAACCTGTGCGTGCAGCACACGCCTACCGA |
| | GGTGACGCGCTACGAGCGCGACGCCGGCGGCCGCCTGCTCGAAGTCGCGAGCGTG |
| | CCGACCGCGGCCGGACTGGCGCTCGGCATCGCGCCCGACACGCTGACCTTCGAAT |
| | ACGACAAGGCCGGGCGGCTGAGCGCCGAACACGGCGCGAACGGCAGCGTCCAGT |
| | ACACGCTCGACGCGCTCGACAACGTGTTGAAGCTCGCCTTGCCGCACGAACAGAC |
| | GCTGCAGATGCTGCGCTACGGCTCGGGGCACGTGCACCAGATTCGCCACGGCGAC |
| | CAGGTCGTCAGCGATTTCGAGCGCGACGACCTGCATCGCGAGTTGACGCGCACGC |
| | AGGGCCCCCTGACCGAGCGGACCGCCTACGACCTGCTGGGCCGCAAGATCTGGCA |
| | ATCAGCCGGCTTCCAGCCCGACGCGCTTGCGCGTGGGCAGGGCCAGCTGTGGCGC |
| | AACTACGGCTACGACGCCGCCGGGGAACTGGTCGAGAGCCACGACAGCCTGCGCG |
| | GCAGCACGCAGTTCAGCTACGATCCGGCCGGCTATCTGACGCAGCGCGTGAACAC |
| | CGCCGACCGGCAGCTCGAATCGTTCGCCTGGGACGCCGCCGGCAACCTGCTCGAC |
| | GATGCGCAACGCAGCAGCCGCGGCTATGTCGAGGGCAACCGGCTGCGCATGTGGC |
| | AGAACCTGCGCTTCGACTACGACGCGTTCGGCAATCTCGCGACCAAGCTGCGCGG |
| | CGCGAATCAGCGCCAGCAGTTCACGTACGATGGGCAGGATCGGCTCGTGGCCGTG |
| | CGCACGCAGGGCGCGCGCGGCGTGGTGGAGACGCGTTTCGCCTACGATCCGCTCG |
| | GGCGGCGCATCGCCAAGACCGATAGGACACTCGACGTGCGCGGCGTAACGCTGCG |
| | CGAGGAAACGAAGCGGTTCGTATGGGAAGGGCTGCGGCTCGCGCAGGAGGTGCG |
| | CGACACCGGCGTGAGCAGCTACGTGTACAGCCCGGATGCGCCTTACATGCCCGCG |
| | GCGCGGGTCGATGCGGTGAAAGCCGAAGCGCTCGCAAACGCCGCGATCGACAAG |
| | GCCAGACAGGCGACGCGGATCTATCACTTTCATACCGATGTGTCGGGCGCACCGC |
| | AAGAAGCGACGAACGAGGCCGGCGACATTGTTTGGGCCGGCCAATACTCAGCCTG |
| | GGGCAAGGTGGCGCCGAACCAGCATGCCCCAGCCCGGATCGATCAGCCGCTCCGC |
| | TACGCCGGACAATATGCCGATGACAGTACCGAGCTGCACTACAACACGTTTCGTTT |
| | CTACGATCCGGATGTCGGCCGGTTTATCAATCAGGATCCAATCGGGTTGATGGGGG |
| | GGCTGAATCTTTACCAATATGCACCCAACTCAATCGCGTGGACCGACTGGTGGGG |
| | GCTGGCCGGCAGCTATACGCTCGGTTCCTATCAAATTTCTGCTCCTCAACTTCCCGC |
| | CTACAATGGGCAGACTGTTGGGACCTTCTACTATGTAAACGACGCGGGCGGGCTC |
| | GAATCGAGGACATTCTCTTCTGGAGGGCCGACCCCTTATCCAAATTATGCCAATGC |
| | CGGGCACGTGGAAGGCCAGTCCGCACTGTTCATGAGGGATAACGGAATTTCAGAC |
| | GGACTGGTTTTCCACAACAACCCTGAGGGTACTTGCGGATTCTGCGTCAATATGAC |
| | CGAAACGCTTTTGCCTGAAAATTCCAAACTTACCGTCGTTCCGCCCGAGGGCTCGA |
| | TTCCGGTCAAGCGGGGCGCGACGGGCGAAACGAGAACATTTACAGGGAACAGCA |
| | AGTCTCCGAAGTCCCCTGTCAAAGGAGGATGTTGA (SEQ ID NO: 131) |
| DddA homolog in | >AJY63123.1 RHS repeat-associated core domain protein [Burkholderia |
| Burkholderia | glumae LMG 2196 = ATCC 33617] |
| glumae LMG | MYEAARVTDPIEHTSALTGFLVGAVLGIALIAAVAFATFTCGFGVALLAGMAAGIGAQ |
| 2196 | VLLSLGESIGKMFSSQSGAITLGSPNVYVNGKPTAYAMLSSVTCSKHNPTPLVAQGST |
| PROTEIN | NIFINGKPAARKDDKITCGATISDGSHDTYFHGGTQTCLPIDDEVPPWLRTATDWAFAL |
| | AGLVGGLGGLLKEAGGLSRAVMPCAAKFIGGYVLGEAASRYVVGPAINSAIGGMFGN |
| | PVDVTTGRKILLAESETDYVVPSPMPVAIRRFYSSDLDYVGTLGRGWVLPWELRLHAR |
| | DGRLWYTDAQGRESGFPMLQPGHAAFSEADQRYLTCTPDGRYILHDLGETYYDFGHY |
| | EPGSGRIGWVRRIEDQAGQWCQFERDSRGRVREIQTCGGLLAVLDYEPEHGRLAGVS |
| | LVSGDQRRLVVAYGYDEHGQMASVTDANGALVRRFTYADGRMTSHSNALGFTSGYT |
| | WQAVGGAPRVVATHTSEGEAWAFEYDIEGRRTHVRHADGRHAQWRYDAQFQIVEY |
| | LDFDGRRYGLKYNDAGMPVMLTLPGERTVTFEYDDAGRIVAETDPLGRTTKTRYDG |
| | NSRRPVEIIAPDGSAWHAEYDRQGRLLATRDPLDRENRYEYPKALSALPIAHVDALGG |
| | RKTFEWNRLGELVAYTDCSGKTTRNFYDAFGLPLARENALGHRVTFDLRPTGEARRV |
| | TYPDGSTESYEYDAAGLMIRHVGLGGRTQIALRNARGQIVEAVDPAGRRTCYRYDAE |
| | GRLRELQQGHARYAFTYSAGGRLTSETRPDGVRRRFEYGEAGDLAALDIVGAADDAT |
| | ANDRPVRTIRFERDRMGNLCAQHTPTEVTRYTRDTGGRLLEVACVPTAAGLALGIAPD |
| | TLTFEYDKAGRLSAEHGANGSVRYTLDALDNVMKLALPHEQTLQMLRYGSGHVHQI |
| | RCGDQVVSDFERDDLHRELTRTQGRLTERTAYDLLGRKIWQSAGFQPDALARGQGQV |
| | WRNYGYDAAGELAESHDSLRGSTQFSYDPAGYLTQRVNTADRQLESFAWDAAGNLL |
| | DDAQRRSRGYVEGNRLRMWQNLRFEYDPFGNLATKLRGANQRQQFTYDGQDRLVA |
| | VRTQDARGVVETRFAYDPLGRRIAKTDIVRDARGVALREETKRFVWEGLRLAQEVRD |
| | TGVSSYVYSPDAPYTPAARVDAVLAEAMAAAAIEQARQATRIYHFHTDVSGAPQEAT |
| | NEAGDIVWAGQYSAWGKVAPNQHAPARIDQPLRYAGQYADDSTELHYNTFRFYDPD |
| | VGRFINQDPIGLMGGLNLYQYAPNSIAWTDWWGLAGSYTLGSYQISAPQLPAYNGQT |
| | VGTFYYVNGAGGLESRTFSSGGPTPYPNYANAGHVEGQSALFMRDNGISDGLVFHNN |
| | PEGTCGFCVNMTETLLPENSKLTVVPPEGAIPVKRGATGETRTFTGNSKSPKSPVKGEC |
| | [SEQ ID NO: 132] |
| DddA homolog in | >KS03_3390 CP009434.1:65330-69607 Burkholderia glumae LMG 2196 = ATCC |
| Burkholderia | 33617 chromosome II, complete sequence |
| glumae LMG | GTGTACGAAGCGGCCCGCGTCACCGACCCGATCGAACACACCAGCGCGCTGACCG |
| 2196 | GCTTTCTGGTGGGCGCCGTGCTCGGCATTGCCCTGATCGCCGCGGTGGCGTTCGCC |
| DNA | ACCTTCACCTGCGGCTTCGGCGTGGCGCTGCTGGCCGGCATGGCCGCCGGCATCGG |
| | CGCGCAGGTGCTGTTGTCGTTAGGAGAATCGATCGGGAAGATGTTCAGTTCGCAAT |
| | CCGGCGCGATCACGCTCGGCTCGCCGAACGTCTATGTGAACGGCAAGCCGACCGC |
| | CTACGCCATGCTCAGCAGCGTGACGTGCAGCAAGCACAACCCGACGCCGCTCGTC |
| | GCGCAGGGGTCCACCAACATCTTCATCAACGGCAAGCCGGCCGCCCGCAAGGACG |
| | ACAAGATCACCTGCGGCGCGACCATCTCCGACGGCTCGCACGACACCTATTTCCAC |
| | GGCGGCACCCAGACCTGCCTGCCGATCGACGACGAAGTGCCGCCGTGGCTGCGCA |
| | CCGCCACCGACTGGGCGTTCGCGCTGGCCGGGCTGGTGGGCGGGCTCGGCGGCCT |
| | GCTCAAGGAAGCGGGCGGGCTGTCGCGCGCGGTGATGCCGTGCGCGGCGAAGTTC |
| | ATCGGCGGCTACGTGCTCGGCGAGGCGGCGAGCCGCTACGTGGTCGGCCCGGCCA |
| | TCAACAGCGCGATCGGCGGGATGTTCGGCAACCCGGTGGACGTCACCACCGGGCG |
| | CAAGATCCTGCTGGCGGAATCGGAAACCGATTACGTGGTGCCCAGCCCGATGCCG |
| | GTGGCGATCCGGCGCTTCTATTCGAGCGACCTCGACTACGTCGGCACGCTCGGGCG |
| | CGGCTGGGTGCTGCCGTGGGAACTGCGGCTGCACGCGCGCGACGGGCGGCTCTGG |
| | TACACCGACGCGCAGGGGCGCGAGAGCGGCTTCCCGATGCTCCAGCCGGGCCATG |
| | CCGCGTTCAGCGAGGCCGACCAGCGCTATCTGACCTGCACCCCGGATGGCCGCTA |
| | CATCCTGCACGACCTCGGCGAAACCTATTACGACTTCGGCCACTACGAGCCGGGCT |
| | CGGGCCGCATCGGCTGGGTGCGCCGCATCGAGGATCAGGCCGGCCAGTGGTGCCA |
| | GTTCGAGCGCGACAGCCGCGGCCGCGTGCGCGAAATCCAGACCTGCGGCGGCTTG |
| | CTGGCCGTGCTCGATTACGAGCCGGAACACGGGCGGCTCGCCGGGGTGTCGCTCG |
| | TCAGCGGGGATCAGCGCCGCCTCGTGGTGGCTTACGGCTATGACGAGCACGGCCA |
| | GATGGCGTCCGTGACCGATGCGAACGGCGCGCTGGTGCGCCGCTTCACCTATGCC |
| | GACGGGCGCATGACGAGCCATTCGAACGCGCTCGGCTTCACGTCGGGCTATACGT |
| | GGCAAGCCGTCGGCGGCGCGCCGCGGGTGGTTGCCACCCACACCAGCGAGGGCGA |
| | GGCCTGGGCCTTCGAGTACGACATTGAAGGACGCCGCACCCACGTGCGTCACGCC |
| | GACGGCCGCCACGCGCAATGGCGCTACGACGCGCAATTCCAGATCGTCGAGTACC |
| | TCGATTTCGACGGCCGGCGCTACGGGCTCAAGTACAACGACGCCGGCATGCCCGT |
| | GATGCTGACGCTGCCCGGCGAACGGACCGTGACGTTCGAGTACGACGATGCCGGC |
| | CGCATCGTCGCCGAAACCGATCCACTCGGCCGCACCACGAAAACGCGCTACGACG |
| | GCAACAGCAGGCGGCCCGTCGAGATCATCGCGCCCGACGGCAGCGCCTGGCACGC |
| | CGAATACGACCGGCAAGGCCGGCTGCTCGCCACCCGCGATCCGCTCGACCGGGAA |
| | AACCGCTACGAATACCCGAAGGCGCTCAGCGCGCTGCCGATCGCGCACGTCGATG |
| | CGCTGGGCGGGCGCAAGACGTTCGAGTGGAACCGGCTCGGCGAGCTGGTGGCCTA |
| | TACCGATTGCTCGGGCAAGACCACACGCAATTTTTACGACGCATTCGGTCTGCCGC |
| | TCGCGCGCGAGAACGCGCTCGGCCACCGCGTGACGTTCGACCTGCGCCCGACCGG |
| | CGAGGCGCGGCGCGTCACCTATCCCGACGGCAGTACAGAAAGCTACGAATACGAC |
| | GCCGCCGGGCTGATGATCCGGCACGTCGGGCTGGGCGGCCGGACGCAGATTGCGC |
| | TGCGCAACGCGCGTGGGCAGATCGTGGAGGCGGTCGATCCGGCCGGACGGCGCAC |
| | CTGCTACCGCTACGACGCCGAGGGGCGGCTGCGCGAGCTGCAACAGGGGCACGCG |
| | CGTTACGCGTTCACCTACAGCGCGGGCGGGCGGCTCACCAGCGAAACCCGGCCCG |
| | ACGGCGTGCGGCGCCGCTTCGAATACGGCGAGGCCGGCGATCTGGCGGCGCTCGA |
| | CATCGTCGGCGCGGCCGACGACGCCACGGCGAACGATCGTCCGGTTCGCACCATC |
| | CGCTTCGAGCGCGACCGCATGGGCAATCTGTGCGCGCAGCACACGCCCACCGAGG |
| | TGACGCGCTACACGCGCGACACCGGCGGCCGCCTGCTCGAAGTCGCATGCGTGCC |
| | GACCGCGGCCGGGCTGGCGCTCGGCATCGCGCCCGACACGCTGACCTTCGAATAC |
| | GACAAGGCCGGGCGGCTGAGTGCCGAACACGGCGCGAACGGCAGCGTCCGATAC |
| | ACGCTCGACGCGCTCGACAACGTGATGAAGCTCGCCCTGCCGCACGAGCAGACGC |
| | TGCAGATGCTGCGCTACGGCTCGGGGCACGTGCATCAGATCCGCTGCGGCGACCA |
| | GGTGGTCAGCGATTTCGAGCGCGACGACCTGCATCGCGAGCTGACGCGCACTCAG |
| | GGCCGCCTGACCGAGCGTACCGCCTACGACCTGCTGGGCCGCAAGATCTGGCAAT |
| | CGGCCGGCTTCCAGCCCGACGCGCTTGCGCGCGGGCAGGGCCAGGTGTGGCGCAA |
| | CTACGGCTACGACGCCGCCGGCGAACTGGCCGAGAGCCACGATAGCCTGCGCGGC |
| | AGCACGCAGTTCAGCTACGATCCGGCCGGCTATCTGACGCAGCGCGTCAATACCG |
| | CCGACCGGCAGCTCGAATCGTTCGCCTGGGATGCCGCCGGCAACCTGCTCGACGA |
| | TGCGCAGCGCCGCAGCCGCGGTTATGTCGAGGGCAACCGGCTGCGCATGTGGCAG |
| | AACCTGCGCTTCGAATACGACCCGTTCGGCAATCTCGCGACCAAGCTGCGCGGCG |
| | CGAACCAGCGCCAGCAGTTCACTTACGACGGGCAGGATCGGCTCGTGGCGGTGCG |
| | CACGCAGGACGCGCGCGGCGTGGTGGAGACGCGTTTCGCCTACGATCCGCTGGGG |
| | CGGCGCATCGCCAAGACGGATATTGTGCGCGACGCGCGCGGCGTAGCGCTGCGCG |
| | AGGAAACGAAGCGGTTCGTGTGGGAGGGGCTGCGGCTCGCGCAGGAGGTGCGCG |
| | ACACGGGCGTGAGCAGCTACGTGTACAGCCCGGACGCGCCCTATACGCCCGCGGC |
| | GCGCGTGGATGCCGTGCTGGCCGAGGCCATGGCCGCCGCTGCCATCGAGCAGGCC |
| | AGACAGGCGACGCGGATCTATCACTTTCATACCGATGTGTCGGGCGCACCGCAAG |
| | AAGCGACGAACGAGGCTGGCGACATTGTTTGGGCCGGCCAATACTCAGCCTGGGG |
| | CAAGGTGGCGCCGAACCAGCATGCCCCCGCCCGGATCGATCAGCCGCTCCGCTAC |
| | GCCGGACAATATGCCGACGACAGTACCGAGCTGCACTACAACACGTTTCGTTTCTA |
| | CGATCCGGACGTCGGCCGGTTTATCAATCAGGATCCAATCGGGTTGATGGGGGGG |
| | CTGAATCTTTACCAATATGCACCCAACTCGATCGCATGGACCGACTGGTGGGGGCT |
| | GGCCGGCAGCTATACGCTCGGTTCCTATCAAATTTCTGCGCCTCAACTTCCGGCCT |
| | ACAATGGACAGACTGTTGGGACCTTCTACTACGTGAACGGCGCGGGCGGGCTCGA |
| | ATCGAGGACATTCTCTTCCGGAGGGCCGACCCCTTATCCAAATTATGCCAATGCCG |
| | GGCACGTGGAGGGCCAGTCCGCGCTGTTCATGAGGGATAACGGAATTTCAGACGG |
| | ACTGGTTTTCCACAACAACCCTGAGGGCACTTGCGGATTCTGCGTTAATATGACCG |
| | AAACGCTTTTGCCTGAAAATTCCAAACTTACCGTCGTTCCGCCCGAGGGCGCGATC |
| | CCGGTCAAGCGGGGCGCGACGGGCGAAACGAGAACATTTACGGGGAACAGCAAG |
| | TCTCCGAAGTCCCCTGTCAAAGGAGAATGTTGA [SEQ ID NO: 133] |
| DddA homolog in | >ACR30728.1 Rhs family protein [Burkholderia glumae BGR1] |
| Burkholderia | MYEAARVTDPIEHTSALTGFLVGAVLGIALIAAVAFATFTCGFGVALLAGMAAGIGAQ |
| glumae BGR1 | VLLSLGESIGKMFSSQSGAITLGSPNVYVNGKPTAYAMLSSVTCSKHNPTPLVAQGST |
| PROTEIN | NIFINGKPAARKDDKITCGATISDGSHDTYFHGGTQTCLPIDDEVPPWLRTATDWAFAL |
| | AGLVGGLGGLLKEAGGLSRAVMPCAAKFIGGYVLGEAASRYVVGPAINSAIGGMFGN |
| | PVDVTTGRKILLAESETDYVVPSPMPVAIRRFYSSDLDYVGTLGRGWVLPWELRLHAR |
| | DGRLWYTDAQGRESGFPMLQPGHAAFSEADQRYLTCTPDGRYILHDLGETYYDFGHY |
| | EPGSGRIGWVRRIEDQAGQWCQFERDSRGRVREIQTCGGLLAVLDYEPEHGRLAGVS |
| | LVSGDQRRLVVAYGYDEHGQMASVTDANGALVRRFTYADGRMTSHSNALGFTSGYT |
| | WQAVGGAPRVVATHTSEGEAWAFEYDIEGRRTHVRHADGRHAQWRYDAQFQIVEY |
| | LDFDGRRYGLKYNDAGMPVMLTLPGERTVTFEYDDAGRIVAETDPLGRTTKTRYDG |
| | NSRRPVEIIAPDGSAWHAEYDRQGRLLATRDPLDRENRYEYPKALSALPIAHVDALGG |
| | RKTFEWNRLGELVAYTDCSGKTTRNFYDAFGLPLARENALGHRVTFDLRPTGEARRV |
| | TYPDGSTESYEYDAAGLMIRHVGLGGRTQIALRNARGQIVEAVDPAGRRTCYRYDAE |
| | GRLRELQQGHARYAFTYSAGGRLTSETRPDGVRRRFEYGEAGDLAALDIVGAADDAT |
| | ANDRPVRTIRFERDRMGNLCAQHTPTEVTRYTRDTGGRLLEVACVPTAAGLALGIAPD |
| | TLTFEYDKAGRLSAEHGANGSVRYTLDALDNVMKLALPHEQTLQMLRYGSGHVHQI |
| | RCGDQVVSDFERDDLHRELTRTQGRLTERTAYDLLGRKIWQSAGFQPDALARGQGQV |
| | WRNYGYDAAGELAESHDSLRGSTQFSYDPAGYLTQRVNTADRQLESFAWDAAGNLL |
| | DDAQRRSRGYVEGNRLRMWQNLRFEYDPFGNLATKLRGANQRQQFTYDGQDRLVA |
| | VRTQDARGVVETRFAYDPLGRRIAKTDIVRDARGVALREETKRFVWEGLRLAQEVRD |
| | TGVSSYVYSPDAPYTPAARVDAVLAEAMAAAAIEQARQATRIYHFHTDVSGAPQEAT |
| | NEAGDIVWAGQYSAWGKVAPNQHAPARIDQPLRYAGQYADDSTELHYNTFRFYDPD |
| | VGRFINQDPIGLMGGLNLYQYAPNSIAWTDWWGLAGSYTLGSYQISAPQLPAYNGQT |
| | VGTFYYVNGAGGLESRTFSSGGPTPYPNYANAGHVEGQSALFMRDNGISDGLVFHNN |
| | PEGTCGFCVNMTETLLPENSKLTVVPPEGAIPVKRGATGETRTFTGNSKSPKSPVKGEC |
| | [SEQ ID NO: 132] |
| DddA homolog in | >bglu_2g02600 NC_012721.2:303868-308145 Burkholderia glumae BGR1 |
| Burkholderia | chromosome 2, complete sequence |
| glumae BGR1 | GTGTACGAAGCGGCCCGCGTCACCGACCCGATCGAACACACCAGCGCGCTGACCG |
| DNA | GCTTTCTGGTGGGCGCCGTGCTCGGCATTGCCCTGATCGCCGCGGTGGCGTTCGCC |
| | ACCTTCACCTGCGGCTTCGGCGTGGCGCTGCTGGCCGGCATGGCCGCCGGCATCGG |
| | CGCGCAGGTGCTGTTGTCGTTAGGAGAATCGATCGGGAAGATGTTCAGTTCGCAAT |
| | CCGGCGCGATCACGCTCGGCTCGCCGAACGTCTATGTGAACGGCAAGCCGACCGC |
| | CTACGCCATGCTCAGCAGCGTGACGTGCAGCAAGCACAACCCGACGCCGCTCGTC |
| | GCGCAGGGGTCCACCAACATCTTCATCAACGGCAAGCCGGCCGCCCGCAAGGACG |
| | ACAAGATCACCTGCGGCGCGACCATCTCCGACGGCTCGCACGACACCTATTTCCAC |
| | GGCGGCACCCAGACCTGCCTGCCGATCGACGACGAAGTGCCGCCGTGGCTGCGCA |
| | CCGCCACCGACTGGGCGTTCGCGCTGGCCGGGCTGGTGGGCGGGCTCGGCGGCCT |
| | GCTCAAGGAAGCGGGCGGGCTGTCGCGCGCGGTGATGCCGTGCGCGGCGAAGTTC |
| | ATCGGCGGCTACGTGCTCGGCGAGGCGGCGAGCCGCTACGTGGTCGGCCCGGCCA |
| | TCAACAGCGCGATCGGCGGGATGTTCGGCAACCCGGTGGACGTCACCACCGGGCG |
| | CAAGATCCTGCTGGCGGAATCGGAAACCGATTACGTGGTGCCCAGCCCGATGCCG |
| | GTGGCGATCCGGCGCTTCTATTCGAGCGACCTCGACTACGTCGGCACGCTCGGGCG |
| | CGGCTGGGTGCTGCCGTGGGAACTGCGGCTGCACGCGCGCGACGGGCGGCTCTGG |
| | TACACCGACGCGCAGGGGCGCGAGAGCGGCTTCCCGATGCTCCAGCCGGGCCATG |
| | CCGCGTTCAGCGAGGCCGACCAGCGCTATCTGACCTGCACCCCGGATGGCCGCTA |
| | CATCCTGCACGACCTCGGCGAAACCTATTACGACTTCGGCCACTACGAGCCGGGCT |
| | CGGGCCGCATCGGCTGGGTGCGCCGCATCGAGGATCAGGCCGGCCAGTGGTGCCA |
| | GTTCGAGCGCGACAGCCGCGGCCGCGTGCGCGAAATCCAGACCTGCGGCGGCTTG |
| | CTGGCCGTGCTCGATTACGAGCCGGAACACGGGCGGCTCGCCGGGGTGTCGCTCG |
| | TCAGCGGGGATCAGCGCCGCCTCGTGGTGGCTTACGGCTATGACGAGCACGGCCA |
| | GATGGCGTCCGTGACCGATGCGAACGGCGCGCTGGTGCGCCGCTTCACCTATGCC |
| | GACGGGCGCATGACGAGCCATTCGAACGCGCTCGGCTTCACGTCGGGCTATACGT |
| | GGCAAGCCGTCGGCGGCGCGCCGCGGGTGGTTGCCACCCACACCAGCGAGGGCGA |
| | GGCCTGGGCCTTCGAGTACGACATTGAAGGACGCCGCACCCACGTGCGTCACGCC |
| | GACGGCCGCCACGCGCAATGGCGCTACGACGCGCAATTCCAGATCGTCGAGTACC |
| | TCGATTTCGACGGCCGGCGCTACGGGCTCAAGTACAACGACGCCGGCATGCCCGT |
| | GATGCTGACGCTGCCCGGCGAACGGACCGTGACGTTCGAGTACGACGATGCCGGC |
| | CGCATCGTCGCCGAAACCGATCCACTCGGCCGCACCACGAAAACGCGCTACGACG |
| | GCAACAGCAGGCGGCCCGTCGAGATCATCGCGCCCGACGGCAGCGCCTGGCACGC |
| | CGAATACGACCGGCAAGGCCGGCTGCTCGCCACCCGCGATCCGCTCGACCGGGAA |
| | AACCGCTACGAATACCCGAAGGCGCTCAGCGCGCTGCCGATCGCGCACGTCGATG |
| | CGCTGGGCGGGCGCAAGACGTTCGAGTGGAACCGGCTCGGCGAGCTGGTGGCCTA |
| | TACCGATTGCTCGGGCAAGACCACACGCAATTTTTACGACGCATTCGGTCTGCCGC |
| | TCGCGCGCGAGAACGCGCTCGGCCACCGCGTGACGTTCGACCTGCGCCCGACCGG |
| | CGAGGCGCGGCGCGTCACCTATCCCGACGGCAGTACAGAAAGCTACGAATACGAC |
| | GCCGCCGGGCTGATGATCCGGCACGTCGGGCTGGGCGGCCGGACGCAGATTGCGC |
| | TGCGCAACGCGCGTGGGCAGATCGTGGAGGCGGTCGATCCGGCCGGACGGCGCAC |
| | CTGCTACCGCTACGACGCCGAGGGGCGGCTGCGCGAGCTGCAACAGGGGCACGCG |
| | CGTTACGCGTTCACCTACAGCGCGGGCGGGCGGCTCACCAGCGAAACCCGGCCCG |
| | ACGGCGTGCGGCGCCGCTTCGAATACGGCGAGGCCGGCGATCTGGCGGCGCTCGA |
| | CATCGTCGGCGCGGCCGACGACGCCACGGCGAACGATCGTCCGGTTCGCACCATC |
| | CGCTTCGAGCGCGACCGCATGGGCAATCTGTGCGCGCAGCACACGCCCACCGAGG |
| | TGACGCGCTACACGCGCGACACCGGCGGCCGCCTGCTCGAAGTCGCATGCGTGCC |
| | GACCGCGGCCGGGCTGGCGCTCGGCATCGCGCCCGACACGCTGACCTTCGAATAC |
| | GACAAGGCCGGGCGGCTGAGTGCCGAACACGGCGCGAACGGCAGCGTCCGATAC |
| | ACGCTCGACGCGCTCGACAACGTGATGAAGCTCGCCCTGCCGCACGAGCAGACGC |
| | TGCAGATGCTGCGCTACGGCTCGGGGCACGTGCATCAGATCCGCTGCGGCGACCA |
| | GGTGGTCAGCGATTTCGAGCGCGACGACCTGCATCGCGAGCTGACGCGCACTCAG |
| | GGCCGCCTGACCGAGCGTACCGCCTACGACCTGCTGGGCCGCAAGATCTGGCAAT |
| | CGGCCGGCTTCCAGCCCGACGCGCTTGCGCGCGGGCAGGGCCAGGTGTGGCGCAA |
| | CTACGGCTACGACGCCGCCGGCGAACTGGCCGAGAGCCACGATAGCCTGCGCGGC |
| | AGCACGCAGTTCAGCTACGATCCGGCCGGCTATCTGACGCAGCGCGTCAATACCG |
| | CCGACCGGCAGCTCGAATCGTTCGCCTGGGATGCCGCCGGCAACCTGCTCGACGA |
| | TGCGCAGCGCCGCAGCCGCGGTTATGTCGAGGGCAACCGGCTGCGCATGTGGCAG |
| | AACCTGCGCTTCGAATACGACCCGTTCGGCAATCTCGCGACCAAGCTGCGCGGCG |
| | CGAACCAGCGCCAGCAGTTCACTTACGACGGGCAGGATCGGCTCGTGGCGGTGCG |
| | CACGCAGGACGCGCGCGGCGTGGTGGAGACGCGTTTCGCCTACGATCCGCTGGGG |
| | CGGCGCATCGCCAAGACGGATATTGTGCGCGACGCGCGCGGCGTAGCGCTGCGCG |
| | AGGAAACGAAGCGGTTCGTGTGGGAGGGGCTGCGGCTCGCGCAGGAGGTGCGCG |
| | ACACGGGCGTGAGCAGCTACGTGTACAGCCCGGACGCGCCCTATACGCCCGCGGC |
| | GCGCGTGGATGCCGTGCTGGCCGAGGCCATGGCCGCCGCTGCCATCGAGCAGGCC |
| | AGACAGGCGACGCGGATCTATCACTTTCATACCGATGTGTCGGGCGCACCGCAAG |
| | AAGCGACGAACGAGGCTGGCGACATTGTTTGGGCCGGCCAATACTCAGCCTGGGG |
| | CAAGGTGGCGCCGAACCAGCATGCCCCCGCCCGGATCGATCAGCCGCTCCGCTAC |
| | GCCGGACAATATGCCGACGACAGTACCGAGCTGCACTACAACACGTTTCGTTTCTA |
| | CGATCCGGACGTCGGCCGGTTTATCAATCAGGATCCAATCGGGTTGATGGGGGGG |
| | CTGAATCTTTACCAATATGCACCCAACTCGATCGCATGGACCGACTGGTGGGGGCT |
| | GGCCGGCAGCTATACGCTCGGTTCCTATCAAATTTCTGCGCCTCAACTTCCGGCCT |
| | ACAATGGACAGACTGTTGGGACCTTCTACTACGTGAACGGCGCGGGCGGGCTCGA |
| | ATCGAGGACATTCTCTTCCGGAGGGCCGACCCCTTATCCAAATTATGCCAATGCCG |
| | GGCACGTGGAGGGCCAGTCCGCGCTGTTCATGAGGGATAACGGAATTTCAGACGG |
| | ACTGGTTTTCCACAACAACCCTGAGGGCACTTGCGGATTCTGCGTTAATATGACCG |
| | AAACGCTTTTGCCTGAAAATTCCAAACTTACCGTCGTTCCGCCCGAGGGCGCGATC |
| | CCGGTCAAGCGGGGCGCGACGGGCGAAACGAGAACATTTACGGGGAACAGCAAG |
| | TCTCCGAAGTCCCCTGTCAAAGGAGAATGTTGA [SEQ ID NO: 133] |
| DddA homolog in | >AOT60363.1 tRNA nuclease WapA precursor [Streptomyces rubrolavendulae] |
| Streptomyces | MSSSDAGRAFGVPENVLARFTRYPGGARRRAGRTARARRLGIVLSAVLSATLLPAEA |
| rubrolavendulae | WAIAPPAPRTGPTLDALQQEEEVDPDPAAMEELDDWDGGPVEPPADYTPTEVTPPTG |
| PROTEIN | GTAPVPLDSAGEELVPAGTLPVRIGQASPTEEDPAPPAPSGTWDVTVEPRATTEAAAV |
| | DGAIIKLTPPASGSTPVDVELDYGRFEDLFGTEWSSRLKLTQLPECFLTTPELEECGTPIT |
| | IPTSNDPATGTVRATVDPADGQPQGLAAQSGGGPAVLAATDSASGAGGTYKATSLSA |
| | TGSWTAGGSGGGFSWSYPLTIPDTPAGPAPKISLSYSSQSVDGRTSVANGQASWIGDG |
| | WDYHPGFVERRYRSCNDDRSGTPNNDNSADKEKSDLCWASDNVVMSLGGSTTELVR |
| | DDTTGTWVAQNDTGARIEYKDKDGGALAAQTAGYDGEHWVVTTRDGTRYWFGRN |
| | TLPGRGAPTNSALTVPVFGNHTGEPCHAATYAASSCTQAWRWNLDYVEDVHGNAM |
| | VVDWKKEQNRYAKNEKFKAAVSYDRDAYPTQILYGLRADDLAGPPAGKVVFHAAPR |
| | CLESAATCSEAKFESKNYADKQPWWDTPATLHCKAGDENCYVTSPTFWSRVRLSAIE |
| | TQGQRTPGSTALSTVDRWTLHQSFPKQRTDTHPPLWLESITRVGFGRPDASGNQSSKA |
| | LPAVTFLPNKVDMPNRVLKSTTDQTPDFDRLRVEVIRTETGGETHVTYSAPCPVGGTR |
| | PTPASNGTRCFPVHWSPDPAAFSDENLDKSGYEPPLEWFNKYVVTKVTEMDLVAEQP |
| | SVETVYTYEGDAAWAKNTDEYGKPALRTYDQWRGYASVVTRTGTTANTGAADATE |
| | QSQTRTRYFRGMSGDAGRAKVHVTLTDVTGTATTVEDLLPYQGMAAETLTYTKAGG |
| | DVAARELAFPYSRKTASRARPGLPALEAYRTGTTRTDSIQHISGDRTRAAQNHTTYDD |
| | AYGLPTQTYSLTLSPNDSGTLVAGDERCTVTTYVHNTAAHIIGLPDRVRATTGDCAAA |
| | PNATTGQIVSDSRTAYDALGAFGTAPVKGLPVQVDTISGGGTSWITSARTEYDALGRA |
| | TKVTDAAGNSTTTTYSPATGPAFEVTVTNAAGHATTTTLDPGRGSALTVTDQNGRKT |
| | TSTYDELGRATGVWTPSRPVNQDASVRFVYQIEDSKVPAVHTRVLRDAGTYEESIELY |
| | DGFLRPRQTQREALGGGRIVTETLYNANGSAKEVRDGYLAEGEPARELFVPLSLDQVP |
| | SATRTAYDGLGRPVRTTTLHRGVPRHSATTAYGGDWELSRTGMSPDGTTPLSGSRAV |
| | KATTDALGRPARIQHFTTQNVSAESVDTTYTYDPRGPLAQVTDAQQNTWTYTYDARG |
| | RKTSSTDPDAGAAYFGYNALDQQVWSKDNQGRLQYTTYDVLGRQTELRDDSASGPL |
| | VAKWTFDTLPGAKGHPVASTRYNDGAAFTSEVTGYDTEYRPTGNKVTIPSTPMTTGL |
| | AGTYTYASTYTPTGKVQSVDLPATPGGLAAEKVITRYDGEDSPTTMSGLAWYTADTF |
| | LGPYGEVLRTASGEAPRRVWTTNVYDEDTRRLTRTTAHRETAPHPVSTTTYGYDTVG |
| | NITSIADQQPAGTEEQCFSYDPMGRLVHAWTDGNSAVCPRTSTAPGAGPARADVSAG |
| | VDGGGYWHSYAFDAIGNRTKLTVHDRTDAALDDTYTYTYGKTLPGNPQPVQPHTLT |
| | QVDAVLNEPGSRVEPRSTYAYDTSGNTTQRVIGGDTQTLAWDRRNKLTSVDTNNDGT |
| | PDVKYLYDASGNRLVEDDGTTRTLFLGEAEIVVNTAGQAVDARRYYSSPGAPTTIRTT |
| | GGKTTGHKLTVMLSDHHSTATTAVELTDTQPVTRRRFDPYGNPRGTEPTTWPDRRTY |
| | LGVGIDDPATGLTHIGAREYDASTGRFISVDPVMDLTDPLQMNGYTYANADPINNSDP |
| | TGLLLDARGGGTQKCVGTCVKDVTNRKGIPLPPGEEWKHEGEAQTDFNGDGFITVFP |
| | TVNVPAKWKKAKKYTEAFYKAVDTACFYGRESCADPEYPSRAHSINNWKGKACKAV |
| | GGKCPERLSWGEGPAFAGGFAIAAEEYAGRGGYRGGGARRGSPCKCFLAGTEVLMA |
| | DGSTKSIEDIKLGDEVVATDPVTGEAGAHPVSALIATENDKRFNELVIITSEGVERLTAT |
| | HEHPFWSPSEGEWLEAGELRTGMTLRSDSGETLVVAGNRAFTQRARTYNLTVADLHT |
| | YYVLAGQTPVLVHNANCGPHLKDLQKDYPRRTVGILDVGTDQLPMISGPGGQSGLLK |
| | NLPGRTKANGEHVETHAAAFLRMNPGVRKAVLYIDYPTGTCGTCRSTLPDMLPEGVQ |
| | LWVISPRRTEKFTGLPD [SEQ ID NO: 134] |
| DddA homolog in | >A4G23_03234 CP017316.1:3756245-3763321 Streptomyces rubrolavendulae |
| Streptomyces | strain MJM4426, complete genome |
| rubrolavendulae | ATGTCCTCGTCCGATGCGGGACGCGCCTTCGGCGTGCCCGAAAACGTCCTGGCGCG |
| DNA | TTTCACGCGGTATCCCGGCGGGGCGCGACGCCGTGCCGGGCGCACGGCGCGCGCC |
| | CGGCGCCTGGGCATCGTGCTGTCCGCCGTCCTCTCGGCGACCCTGCTGCCCGCCGA |
| | GGCATGGGCCATCGCGCCCCCGGCGCCGCGCACCGGTCCGACCCTGGACGCCCTC |
| | CAGCAGGAGGAGGAGGTCGATCCGGACCCGGCCGCCATGGAAGAGCTGGACGAC |
| | TGGGACGGTGGGCCGGTCGAGCCCCCGGCCGACTACACCCCCACCGAGGTCACGC |
| | CTCCCACCGGCGGCACCGCCCCGGTGCCGCTGGACAGCGCGGGCGAGGAACTGGT |
| | CCCGGCCGGGACCCTGCCCGTGCGCATCGGCCAGGCGTCCCCCACCGAGGAGGAC |
| | CCGGCACCCCCGGCACCCAGCGGCACGTGGGACGTCACCGTGGAGCCCCGCGCCA |
| | CCACCGAGGCGGCCGCCGTGGACGGCGCCATCATCAAGCTCACCCCGCCCGCCAG |
| | CGGCTCCACACCGGTCGACGTGGAACTCGACTACGGCCGGTTCGAGGACCTGTTC |
| | GGCACCGAGTGGTCCTCCCGGCTCAAGCTGACGCAGCTCCCGGAGTGCTTCCTCAC |
| | GACGCCCGAGCTGGAGGAGTGCGGCACCCCCATCACCATCCCGACGAGCAACGAC |
| | CCGGCCACCGGGACGGTCCGGGCCACCGTCGACCCGGCCGACGGGCAGCCGCAGG |
| | GCCTGGCCGCGCAGTCGGGCGGCGGTCCCGCCGTCCTCGCCGCGACCGACTCGGC |
| | GTCCGGCGCCGGCGGCACGTACAAGGCGACCTCCCTCTCGGCCACCGGCTCCTGG |
| | ACGGCCGGCGGCAGCGGCGGCGGCTTCTCCTGGTCGTATCCGCTCACCATCCCGGA |
| | CACCCCGGCCGGCCCCGCGCCGAAGATCTCCCTGTCGTACTCCTCCCAGTCCGTCG |
| | ACGGCCGCACCTCCGTCGCCAACGGCCAGGCGTCGTGGATAGGCGACGGCTGGGA |
| | CTACCACCCCGGCTTCGTCGAGCGCCGCTACCGCTCCTGCAACGACGACCGCTCCG |
| | GCACCCCGAACAACGACAACAGTGCGGACAAGGAGAAGTCCGACCTGTGCTGGGC |
| | GAGCGACAACGTCGTGATGTCGCTCGGCGGCTCCACCACCGAACTCGTCCGCGAC |
| | GACACGACCGGCACGTGGGTCGCGCAGAACGACACCGGTGCCCGGATCGAGTACA |
| | AGGACAAGGACGGCGGAGCCCTGGCCGCCCAGACCGCCGGCTACGACGGCGAGC |
| | ACTGGGTCGTCACCACCCGCGACGGAACCCGCTACTGGTTCGGCCGCAACACCCTC |
| | CCCGGCCGCGGCGCCCCCACGAACTCCGCCCTCACCGTCCCCGTCTTCGGCAACCA |
| | CACCGGCGAGCCCTGCCACGCCGCCACCTACGCCGCCTCCTCCTGCACCCAGGCGT |
| | GGCGCTGGAACCTCGACTACGTCGAGGACGTCCACGGCAACGCGATGGTCGTCGA |
| | CTGGAAGAAGGAGCAGAACCGGTACGCGAAGAACGAGAAGTTCAAGGCGGCTGT |
| | CTCCTACGACCGCGACGCGTATCCGACGCAGATCCTCTACGGCCTGCGCGCCGACG |
| | ACCTGGCGGGCCCGCCCGCCGGCAAGGTCGTCTTCCACGCCGCCCCGCGCTGCCTC |
| | GAAAGCGCGGCCACCTGCTCCGAAGCCAAGTTCGAGTCCAAGAACTACGCGGACA |
| | AGCAGCCCTGGTGGGACACACCGGCCACCCTGCACTGCAAGGCCGGTGACGAGAA |
| | CTGCTACGTCACCTCGCCGACGTTCTGGAGCCGCGTCCGCCTGTCGGCGATCGAGA |
| | CGCAGGGTCAGCGCACGCCCGGCTCGACGGCGCTGTCCACGGTCGACCGCTGGAC |
| | CCTGCACCAGTCGTTCCCGAAGCAGCGCACCGACACCCACCCGCCGCTCTGGCTGG |
| | AGTCGATCACCCGCGTGGGCTTCGGCCGGCCGGACGCCTCCGGCAACCAGTCGAG |
| | CAAGGCCCTCCCGGCGGTGACCTTCCTGCCCAACAAGGTCGACATGCCGAACCGC |
| | GTGCTGAAGAGCACGACGGACCAGACGCCCGATTTCGACCGCCTGCGCGTCGAGG |
| | TCATCCGCACGGAGACCGGCGGCGAGACCCATGTGACGTACTCCGCCCCCTGCCC |
| | CGTCGGCGGCACCCGCCCCACCCCGGCCTCCAACGGCACCCGCTGCTTCCCGGTCC |
| | ACTGGTCCCCCGACCCGGCGGCCTTCTCCGACGAGAACCTGGACAAGAGCGGCTA |
| | CGAGCCGCCCCTCGAGTGGTTCAACAAGTACGTCGTCACCAAGGTCACCGAGATG |
| | GACCTCGTGGCGGAGCAGCCCAGCGTCGAGACCGTCTACACCTACGAGGGCGACG |
| | CCGCCTGGGCGAAGAACACCGACGAGTACGGCAAGCCCGCCCTGCGCACCTACGA |
| | CCAGTGGCGCGGCTACGCGAGCGTCGTCACCCGCACGGGCACCACGGCCAACACC |
| | GGCGCCGCCGACGCCACCGAGCAGTCCCAGACCCGCACCCGGTACTTCCGCGGCA |
| | TGTCCGGCGACGCGGGCCGCGCCAAGGTGCACGTCACGCTCACGGACGTGACCGG |
| | CACCGCGACCACCGTCGAGGACCTGCTCCCGTACCAGGGCATGGCCGCCGAGACC |
| | CTTACCTACACCAAGGCGGGCGGCGACGTCGCCGCCCGCGAGCTGGCCTTCCCCTA |
| | CAGCAGGAAGACCGCCTCCCGCGCCCGCCCCGGCCTCCCCGCCCTGGAGGCGTAC |
| | CGCACGGGCACGACGCGCACGGACTCCATCCAGCACATCAGCGGCGACCGGACGC |
| | GCGCCGCTCAGAACCACACCACATACGACGACGCGTACGGCCTGCCCACCCAGAC |
| | CTACTCGCTGACACTCTCGCCGAACGACTCCGGCACCCTTGTCGCCGGTGACGAGC |
| | GGTGCACCGTCACGACGTACGTCCACAACACCGCCGCGCACATCATCGGCCTCCCC |
| | GACCGCGTCCGCGCCACGACGGGCGACTGCGCCGCCGCGCCGAACGCCACCACCG |
| | GCCAGATCGTCTCCGACAGCCGCACCGCGTACGACGCGCTCGGCGCCTTCGGCAC |
| | GGCCCCGGTCAAGGGCCTGCCGGTCCAGGTGGACACGATCTCCGGAGGCGGCACG |
| | AGCTGGATCACCTCGGCGCGCACGGAGTACGACGCGCTGGGCCGTGCGACCAAGG |
| | TCACCGACGCGGCGGGCAACTCCACCACGACCACGTACAGCCCGGCGACCGGCCC |
| | CGCGTTCGAGGTCACCGTGACCAACGCGGCTGGTCATGCCACGACCACCACCCTC |
| | GACCCCGGTCGCGGCTCGGCGCTGACCGTCACCGACCAGAACGGCCGCAAGACCA |
| | CCAGCACGTACGACGAACTCGGCCGGGCCACCGGCGTGTGGACGCCCTCCCGCCC |
| | GGTGAACCAGGACGCGTCCGTGCGCTTCGTCTACCAGATCGAGGACAGCAAGGTC |
| | CCGGCGGTGCACACTCGGGTCCTGCGCGACGCCGGTACGTACGAGGAGTCGATCG |
| | AGCTCTACGACGGCTTCCTCCGCCCCCGTCAGACCCAGCGCGAGGCGCTGGGCGG |
| | CGGCCGAATCGTCACCGAGACCCTCTACAACGCCAACGGCTCTGCGAAGGAAGTG |
| | CGCGACGGCTACCTGGCGGAGGGCGAGCCCGCGCGGGAACTGTTCGTCCCGCTCT |
| | CCCTCGACCAGGTGCCGAGCGCGACGAGGACGGCCTATGACGGCCTGGGCCGGCC |
| | CGTCCGGACGACGACCCTCCACAGGGGAGTCCCCCGGCACTCCGCCACCACGGCG |
| | TACGGCGGCGACTGGGAACTGAGCCGCACCGGCATGTCGCCCGACGGAACGACGC |
| | CGCTCTCTGGCAGCCGCGCCGTGAAGGCGACGACGGACGCGCTCGGCCGCCCGGC |
| | CCGCATCCAGCACTTCACCACCCAGAACGTGTCGGCCGAGAGCGTCGACACCACG |
| | TACACCTACGACCCCCGCGGCCCCCTTGCCCAGGTCACCGACGCCCAGCAGAACA |
| | CCTGGACGTACACGTACGACGCCCGTGGGCGCAAGACGTCCTCCACCGACCCGGA |
| | CGCGGGCGCCGCCTACTTCGGCTACAACGCGCTGGACCAGCAGGTCTGGTCGAAG |
| | GACAACCAGGGCCGCCTGCAGTACACGACGTACGACGTCCTGGGCCGCCAGACCG |
| | AGCTGCGCGACGACTCCGCGTCCGGCCCGCTGGTGGCGAAGTGGACCTTCGACAC |
| | CCTGCCGGGCGCCAAGGGCCACCCGGTCGCGTCGACCCGCTACAACGACGGCGCC |
| | GCGTTCACCAGCGAGGTGACCGGTTACGACACCGAGTACCGTCCGACCGGCAACA |
| | AGGTCACCATCCCCAGCACCCCGATGACCACGGGCCTCGCCGGCACGTACACGTA |
| | CGCCAGCACGTACACCCCGACCGGCAAGGTCCAGTCCGTCGACCTGCCCGCGACG |
| | CCCGGCGGGCTCGCCGCGGAGAAGGTGATCACCCGCTACGACGGCGAGGACTCGC |
| | CCACCACGATGTCGGGCCTGGCCTGGTACACGGCCGACACCTTCCTCGGCCCGTAC |
| | GGGGAAGTGCTGCGCACGGCGTCGGGCGAGGCCCCGCGCCGCGTGTGGACGACCA |
| | ACGTCTACGACGAGGACACCCGCCGCCTCACCAGGACCACCGCGCACCGGGAGAC |
| | GGCTCCCCACCCGGTCAGCACGACCACCTACGGCTACGACACGGTCGGCAACATC |
| | ACGTCCATCGCCGACCAGCAGCCGGCGGGTACCGAGGAGCAGTGCTTCTCGTACG |
| | ACCCGATGGGGCGCCTCGTCCACGCCTGGACGGACGGCAACAGCGCCGTCTGCCC |
| | CAGGACGTCCACGGCACCGGGCGCCGGCCCGGCCCGCGCCGACGTCTCGGCCGGT |
| | GTCGACGGCGGCGGATACTGGCACTCGTACGCGTTCGACGCGATCGGCAACCGGA |
| | CGAAGCTGACCGTCCACGACCGCACCGACGCGGCCCTGGACGACACGTACACCTA |
| | CACCTACGGCAAGACCCTGCCGGGTAACCCGCAGCCGGTCCAGCCGCACACCCTC |
| | ACCCAGGTCGACGCGGTGCTCAACGAGCCCGGATCGAGAGTCGAACCGCGCTCCA |
| | CATACGCCTACGACACCTCCGGCAACACCACCCAGCGCGTCATCGGCGGCGACAC |
| | CCAGACCCTGGCCTGGGACCGCCGCAACAAGCTGACGTCCGTCGACACGAACAAC |
| | GACGGCACACCGGACGTGAAGTACCTGTACGACGCGTCGGGCAACCGCCTGGTCG |
| | AGGACGACGGCACCACGCGCACCCTCTTCCTCGGCGAGGCCGAGATCGTCGTCAA |
| | CACGGCCGGCCAGGCCGTGGACGCGCGCCGCTACTACAGCAGCCCCGGCGCCCCG |
| | ACGACGATCCGCACGACCGGCGGCAAGACCACGGGCCACAAGCTGACCGTCATGC |
| | TGTCGGACCACCACAGCACGGCGACGACCGCGGTCGAGCTGACCGACACCCAGCC |
| | GGTCACCCGCCGCCGCTTCGACCCGTACGGCAACCCCCGCGGCACCGAGCCGACC |
| | ACCTGGCCCGACCGCCGCACCTACCTGGGCGTCGGCATCGACGACCCCGCCACGG |
| | GCCTGACCCACATCGGCGCCCGCGAATACGACGCATCGACGGGCCGCTTCATCTCC |
| | GTCGATCCGGTCATGGACCTCACGGACCCGCTCCAGATGAACGGGTACACCTACG |
| | CCAACGCGGACCCGATCAACAACAGCGACCCCACCGGACTGTTGCTCGACGCCCG |
| | AGGCGGCGGCACTCAGAAGTGCGTGGGAACCTGCGTCAAGGACGTCACGAACCGA |
| | AAGGGAATTCCGCTCCCGCCTGGCGAGGAGTGGAAGCATGAAGGGGAGGCGCAA |
| | ACCGATTTCAACGGTGACGGCTTCATCACCGTCTTCCCGACCGTGAATGTTCCGGC |
| | GAAGTGGAAGAAGGCGAAGAAGTACACGGAGGCTTTCTACAAGGCGGTTGATACT |
| | GCTTGCTTCTATGGACGCGAAAGCTGTGCGGATCCGGAGTACCCTTCGCGGGCGCA |
| | TAGCATCAACAACTGGAAGGGAAAGGCATGCAAAGCCGTAGGGGGAAAATGCCC |
| | TGAGAGGTTGTCGTGGGGGGAGGGTCCGGCGTTCGCTGGTGGCTTCGCGATAGCA |
| | GCGGAAGAGTATGCGGGGAGAGGGGGCTACCGGGGCGGTGGGGCGAGGAGGGGG |
| | TCGCCCTGTAAGTGCTTCCTTGCCGGCACCGAGGTGCTCATGGCGGATGGCAGCAC |
| | TAAAAGTATCGAGGACATCAAGCTCGGTGACGAAGTGGTTGCGACTGATCCGGTA |
| | ACCGGTGAGGCCGGTGCGCACCCTGTCTCGGCGCTGATCGCCACCGAGAACGACA |
| | AGCGTTTCAACGAGCTGGTCATTATCACCAGCGAGGGTGTAGAGCGTCTTACCGCA |
| | ACGCATGAGCACCCCTTCTGGTCGCCATCCGAAGGGGAGTGGTTGGAGGCGGGTG |
| | AGCTGCGCACTGGCATGACGCTGCGCTCCGACTCTGGCGAAACTCTCGTAGTCGCA |
| | GGAAACCGCGCCTTCACCCAGCGAGCCCGGACCTACAACCTCACGGTTGCAGACC |
| | TCCACACGTACTATGTGCTGGCGGGCCAGACTCCGGTACTGGTTCACAATGCAAAC |
| | TGTGGACCTCACCTGAAGGACCTGCAAAAGGACTACCCCCGGCGCACTGTGGGCA |
| | TCCTTGACGTCGGAACTGATCAGCTCCCGATGATTAGCGGCCCAGGTGGCCAGTCG |
| | GGACTTCTCAAGAACCTCCCAGGTCGTACGAAGGCCAACGGGGAGCACGTGGAGA |
| | CTCACGCAGCAGCGTTCTTGCGTATGAACCCGGGTGTCAGAAAGGCCGTGCTCTAC |
| | ATCGACTACCCGACGGGGACCTGCGGAACATGTAGAAGTACATTGCCTGACATGC |
| | TGCCCGAGGGTGTTCAGTTGTGGGTGATCTCGCCGCGTAGGACTGAAAAATTCACG |
| | GGACTTCCTGACTGA [SEQ ID NO: 135] |
| DddA homolog in | >AVT32940.1 hypothetical protein C6361_29650 [Plantactinospora sp. BC1] |
| Plantactinospora | MGDRLPAFVDGGDTLGIFSRGGIERDLASGVAGPASSLPKGTPGFNGLVKSHVEGHAA |
| sp. BC1 | ALMRQNGIPNAELYINRVPCGSGNGCAAMLPHMLPEGATLRVYGPNGYDRTFTGLPD |
| PROTEIN | [SEQ ID NO: 136] |
| DddA homolog in | >C6361_29650 CP028158.1:6764267-6764614 Plantactinospora sp. BC1 |
| Plantactinospora | chromosome, complete genome |
| sp. BC1 | CTGGGTGACCGGCTCCCTGCCTTCGTGGACGGTGGAGACACGTTGGGCATCTTTTC |
| DNA | TCGCGGAGGTATTGAGCGGGACCTCGCCAGCGGAGTTGCGGGTCCTGCAAGTAGC |
| | CTTCCTAAAGGCACGCCTGGCTTCAATGGTCTTGTAAAGAGTCATGTTGAAGGGCA |
| | TGCGGCTGCGCTAATGAGACAAAATGGAATTCCGAACGCTGAGCTGTATATCAAC |
| | AGAGTGCCGTGCGGTTCAGGTAATGGCTGCGCAGCGATGTTGCCGCATATGCTTCC |
| | GGAAGGTGCCACCCTCCGCGTATATGGGCCGAACGGGTACGATAGAACCTTCACT |
| | GGACTTCCGGACTGA [SEQ ID NO: 137] |
| DddA homolog in | >BAJ27137.1 hypothetical protein KSE_13070 [Kitasatospora setae |
| Kitasatospora | KM-6054] |
| setae KM-6054 | MAAVPSAEALAAKRARDTIWTPPNTPLGSQTKSVDGENLVPGRLPGPLEPEPADWTPG |
| PROTEIN | GPASVPAPGSADVTLGFDSAEAAAARKATGGAAPASDGAALRAGSLPVVIGAAKDAK |
| | SGAHRIRVELVDQAKSRAAHLDSPLIALTDTEPDTPPSGRTTKVSLDLKGIGAQTWAD |
| | RARLVALPACALETPDRPECQQQTPVQSSVDLRSGLLTAEVILPAATEGTAPPTKSSLG |
| | SGTASGVVQAGLTTAAPAKAAPTVLAATAGASGSGGSFSATSLSPSAAWGAGSNVGN |
| | FTYSYPIQTPPSLGGTAPSVGLGYDSSAVDGKTSAQNSQSSWLGEGWGYEAGFIERGY |
| | KSCNTAGIANSSDMCWGGQNATLSLAGHSGTLVRDDTTGVWHLQSDDGTKIEQLTG |
| | APNGLQNGEHWRITTTDGTQFYFGRNHLPGGDGTDPASNSAFKEPVYSPKSGDPCYNS |
| | STATGSWCTMGWRWNLDYAVDVHGNLITYTYAQETNYYSRGAGQNSGSGTLTDYT |
| | RAGYLTQIAYGQRLSEQVTAKGAAKAAALITFTAAERCVPSGSITCTEAQRTTANASY |
| | WPDTPLDQVCASTGTCTRAGPTFFTTKRLASLTTQVLVSGAYRTVDTWTLTHSFKDPG |
| | DGNAKSLWLDSIQRTGTNGQTAVTMPPVTFTAVMKPNRVDGDLTLKDGTKVTVTPF |
| | NRPRLQQVTTETGGQINVVYTTSSDAAHPACSRLAGTMPAAADGNTLACAPVKWYLP |
| | GSSSPDPVDDWFNKYLISAVTEQDAISGTTLIKATNYTYNGDAAWHRNDAEFTDAKT |
| | RTWDGFRGYQSVTSTTGSAYPGEAPRTQQTATYLRGMDGDVKADGSTRSVQVANPL |
| | GGPALTDSPWLAGSSFATQTYDQAGGTVISANGSVAGGQQVTATHAQSGGMPALVA |
| | RYPASQVTTTSKSKLSDGTWRTNTTVSTSDPAHANRPLSSDDKGDGTPGAELCSTNGY |
| | ATGTNPMMLNILAERTVTKGACGTPVTSANTVSSARTLYDGKPYGQAGDLAESTSAL |
| | TLDHYDTGGNPVYVHTAASTFDAYGRLTSVSEANGATYDAAGNQLTAPNLTPATTRT |
| | AYTPATGAIATTVTQTTPTGWTTTLTQDPGRAEALVSTDANGRATTQQYDGLGRLTA |
| | AWSPERATNLTPSQKFSYAVNGTTGPSVVTSQWLKEAGGYAYKNELYDGLGRLRQV |
| | QRTSDTYSGRLITDTVYDSHGWPVKTASPYYEKTTAPNSTVYLPQDSQVPAQTWVTF |
| | DGIGRTTRSAFVSYGQQQWATTTAYPGADRTDVTPPNGKYPTSTFTDGRNQVSALWQ |
| | YRTATPTGNPADATVTTYTYDAANRPATRKDAAGNTWSYGYDLRGRQTTVTDPDTG |
| | TTTTAYDVNSRAVSTTDGKGNTLVVSYDLIGRKTGLYQGSIAPANQLAGWTYDTLPG |
| | GKGKPTSSTRYVGGAGGSAYTQAVTGYDAGYRPTGTSVTIPASEGKLAGTYTTGLTY |
| | NPVLGTLKQTDLPAIGAAPAESVMYTYNISGVLQKSYSDTYYVYDVQYDAFGRPVRT |
| | TTGDAGTQVVSTQLDKTDYTYNQAGDVTSVTDVQNGTATDAQCFTYDHLGRLTQA |
| | WTDTAGSTSTTSGTWTDTSGTVHNSGSSQSVPALGACANANGPASTGSPAKLSVGGP |
| | SPYWQSYGYDSTGNRTTLVQHDTTGNTTKDTTTTQTFGPAGSVNTATGAPNTGGGTG |
| | GPHALLTSSTTGPTGTQVTSYQYDQLGNTTAVTETSGTTTLAWNGEDKLASVTKTGQ |
| | AQATSYLYDADGNQLIRRNPGKTTLNLGSDEVTLDTAANSLTDTRYYSAPGGISIART |
| | TGPTGASALAYQASDPHGTANVQINVDAAQTTTRRPTDPFGNPRGTQPAPNTWAGDK |
| | GFVGGTKDDTTGLTNLGAREYQPTTGRFLNPDPLLDAGNPQQWNGYAYSDNDPVNS |
| | SDPSGLITNALADGDTYVARPAAFCVTMSCVEQTSGPGFWEDKRVGDAVFAAVVQA |
| | TTQSNGNGSSQTKKEKGIWGQAWDWTKKNGGAILGALVEGAVFSTCFIGAGFAAPAT |
| | GGITVIAGAAACGAVAGEAGALTTNILTPDADHSVDGITNDMVVGEITGAAVSAASEG |
| | ASSLAKPAVRKLLGMEAEEGLEAAGRAATGPCNSFPAGVTVLLADGTTKPIEQIAQGD |
| | QVTATDPQTGTTQAEPVTDTIIGHDDTEFTDLTLTNDADPRAPPSEITSTTHHPYWNAT |
| | TSRWTDAGDLKPGDHVRTPDGTELTVNTVYSYTTQPRTARNLTVADLHTYYVLAGN |
| | TPVLVHNTGPGCGEPGFVSDAANSLSGRRITTGQIFDASGNPIGPEITSGGGSLADRAQS |
| | YLADSPNIRNLPAKARYASADHVEAQYAVWMRENGVTDASVVINQNYVCGLPLGCQ |
| | AAVPAILPRGSTMTVWYPGSGSPIVLRGVG [SEQ ID NO: 138] |
| DddA homolog in | >KSE_13070 NC_016109.1:1451556-1458878 Kitasatospora setae KM-6054 DNA, |
| Kitasatospora | complete genome |
| setae KM-6054 | GTGCTGGGGACAGCGGCCGCGCTCGCGGTCATGATGTCCATGGCGGCGGTGCCGT |
| DNA | CCGCCGAGGCACTGGCCGCGAAGCGGGCACGCGACACCATCTGGACGCCGCCCAA |
| | CACCCCGCTGGGCAGCCAGACCAAGTCCGTCGACGGCGAGAACCTCGTCCCGGGC |
| | CGCCTGCCCGGCCCCCTGGAGCCGGAACCGGCCGACTGGACACCCGGCGGACCGG |
| | CATCCGTGCCCGCTCCGGGCAGCGCGGACGTCACCCTCGGCTTCGACTCCGCGGAG |
| | GCCGCCGCCGCCCGCAAGGCCACCGGCGGCGCCGCCCCCGCCTCCGACGGCGCGG |
| | CCCTCCGCGCGGGCTCCCTCCCCGTCGTCATCGGCGCGGCGAAGGACGCCAAGAG |
| | CGGCGCCCACCGGATCCGCGTCGAGCTCGTGGACCAGGCCAAGAGCCGTGCCGCA |
| | CACCTCGACAGCCCGCTGATCGCACTCACCGACACCGAGCCGGACACCCCGCCCT |
| | CCGGTCGGACCACGAAGGTGTCCCTCGACCTGAAGGGCATCGGCGCCCAGACCTG |
| | GGCGGACCGCGCGCGACTCGTCGCCCTGCCCGCCTGCGCCCTGGAGACGCCCGAC |
| | AGGCCCGAGTGCCAGCAGCAGACCCCCGTGCAGAGCTCCGTCGACCTGCGCTCCG |
| | GACTGCTGACGGCCGAGGTCATTCTGCCCGCCGCCACCGAGGGCACCGCCCCGCC |
| | CACCAAGAGCTCCCTCGGCTCGGGCACCGCCTCCGGCGTCGTCCAGGCCGGCCTCA |
| | CCACGGCGGCGCCCGCCAAGGCCGCGCCCACGGTGCTCGCCGCGACCGCCGGCGC |
| | GTCCGGCTCGGGCGGCAGCTTCTCGGCGACCTCGCTGTCGCCCTCCGCGGCCTGGG |
| | GCGCCGGCTCCAACGTCGGCAACTTCACCTACTCGTACCCGATCCAGACGCCTCCC |
| | TCGCTCGGCGGGACCGCCCCCTCCGTGGGCCTCGGGTACGACTCGTCCGCCGTCGA |
| | CGGGAAGACCTCCGCGCAGAACTCCCAGTCCTCCTGGCTCGGCGAGGGCTGGGGC |
| | TACGAGGCCGGGTTCATCGAGCGCGGCTACAAGTCCTGCAACACGGCCGGCATCG |
| | CGAACTCCTCGGACATGTGCTGGGGGGGGCAGAACGCCACCCTCTCGCTGGCCGG |
| | CCACTCCGGCACCCTGGTGCGCGACGACACCACCGGCGTCTGGCACCTGCAGAGC |
| | GACGACGGCACGAAGATCGAACAGCTCACCGGCGCGCCCAACGGCCTGCAGAAC |
| | GGCGAGCACTGGCGGATCACCACGACCGACGGCACGCAGTTCTACTTCGGCCGCA |
| | ACCACCTGCCCGGCGGCGACGGCACCGACCCGGCGAGCAACTCCGCCTTCAAGGA |
| | ACCGGTGTACTCGCCCAAGAGCGGCGACCCCTGCTACAACTCCTCCACCGCCACCG |
| | GCTCCTGGTGCACGATGGGCTGGCGCTGGAACCTCGACTACGCCGTCGACGTCCAC |
| | GGCAACCTGATCACCTACACCTACGCCCAGGAGACCAACTACTACAGCCGAGGCG |
| | CCGGCCAGAACAGCGGCAGCGGCACCCTGACCGACTACACCCGCGCCGGCTACCT |
| | CACCCAGATCGCCTACGGCCAGCGCCTGAGCGAGCAGGTCACCGCCAAGGGCGCG |
| | GCCAAGGCCGCTGCCCTCATCACCTTCACCGCCGCGGAACGCTGCGTCCCGTCCGG |
| | CTCGATCACCTGCACCGAGGCACAGCGCACGACCGCGAACGCCTCGTACTGGCCG |
| | GACACCCCGCTCGACCAGGTCTGCGCCTCCACCGGCACCTGCACCCGGGCCGGCC |
| | CGACGTTCTTCACCACCAAGCGCCTCGCCTCCCTCACCACCCAGGTCCTGGTCTCC |
| | GGCGCCTACCGCACCGTCGACACCTGGACGCTCACCCATTCCTTCAAGGACCCGGG |
| | CGACGGCAACGCCAAGTCGCTGTGGCTCGACTCGATCCAGCGCACCGGCACCAAC |
| | GGGCAGACCGCGGTCACCATGCCGCCCGTCACCTTCACGGCGGTGATGAAGCCGA |
| | ACCGGGTGGACGGGGACCTCACCCTCAAGGACGGCACCAAGGTCACCGTCACCCC |
| | GTTCAACCGGCCCCGCCTCCAGCAGGTCACCACGGAGACCGGCGGCCAGATCAAC |
| | GTCGTCTACACCACCTCCTCCGACGCCGCGCACCCCGCCTGCTCGCGCCTGGCCGG |
| | CACCATGCCCGCCGCGGCGGACGGCAACACCCTCGCCTGCGCCCCCGTCAAGTGG |
| | TACCTGCCCGGATCCAGCTCCCCGGACCCGGTCGACGACTGGTTCAACAAGTACCT |
| | GATCAGCGCCGTCACCGAACAGGACGCGATCAGCGGCACCACCCTGATCAAGGCC |
| | ACCAACTACACCTACAACGGCGACGCCGCCTGGCACCGCAACGACGCCGAGTTCA |
| | CCGACGCCAAGACCCGCACCTGGGACGGCTTCCGCGGCTACCAGTCCGTCACCAG |
| | CACCACCGGCAGCGCCTACCCGGGCGAGGCCCCCAGGACCCAGCAGACCGCGACC |
| | TACCTGCGCGGCATGGACGGCGACGTCAAGGCCGACGGCTCCACCCGCAGCGTCC |
| | AGGTCGCCAACCCGCTCGGCGGCCCGGCCCTCACCGACAGCCCGTGGCTGGCCGG |
| | CTCCAGCTTCGCCACCCAGACCTACGACCAGGCCGGCGGCACCGTCATCTCCGCCA |
| | ACGGCTCCGTCGCCGGCGGCCAGCAGGTCACCGCCACCCACGCCCAGAGCGGCGG |
| | CATGCCGGCCCTGGTCGCCCGCTACCCCGCCTCCCAGGTCACCACCACCTCCAAGT |
| | CCAAGCTCTCCGACGGGACCTGGCGCACCAACACCACCGTCAGCACCAGCGACCC |
| | CGCGCACGCCAACCGCCCCCTCAGCAGCGACGACAAGGGCGACGGCACCCCCGGC |
| | GCCGAACTGTGCAGCACCAACGGCTACGCCACCGGCACCAACCCGATGATGCTGA |
| | ACATCCTCGCCGAGCGGACGGTCACCAAGGGCGCCTGCGGCACCCCCGTGACCTC |
| | GGCCAACACCGTCTCCTCCGCCCGCACCCTCTACGACGGCAAGCCCTACGGCCAG |
| | GCCGGCGACCTCGCCGAGTCCACCAGCGCCCTGACCCTGGACCACTACGACACCG |
| | GCGGCAACCCCGTCTACGTCCACACCGCCGCCTCCACCTTCGACGCCTACGGCCGG |
| | CTTACCAGCGTCAGCGAGGCCAACGGCGCCACCTACGACGCCGCGGGCAACCAGC |
| | TCACCGCGCCCAACCTCACCCCCGCCACCACCCGCACCGCCTACACCCCGGCCACC |
| | GGCGCCATCGCCACCACCGTCACCCAGACCACGCCCACCGGCTGGACCACCACCC |
| | TCACCCAGGACCCGGGCCGCGCCGAAGCTCTGGTCTCCACCGACGCCAACGGCCG |
| | CGCCACCACCCAGCAGTACGACGGCCTCGGCCGCCTGACCGCCGCCTGGTCACCG |
| | GAGCGCGCGACCAACCTCACCCCCAGCCAGAAGTTCTCCTACGCGGTCAACGGCA |
| | CCACCGGCCCCTCCGTCGTCACCTCCCAGTGGCTCAAGGAAGCCGGCGGCTACGC |
| | GTACAAGAACGAGCTGTACGACGGCCTCGGCCGCCTGCGCCAGGTCCAGCGCACC |
| | AGCGACACCTACTCCGGGCGGCTGATCACCGACACCGTCTACGACTCGCACGGCT |
| | GGCCCGTCAAGACCGCCAGCCCGTACTACGAGAAGACCACCGCGCCCAACAGCAC |
| | CGTCTACCTGCCGCAGGACTCCCAGGTGCCCGCCCAGACCTGGGTCACCTTCGACG |
| | GCATCGGCCGGACCACCCGCTCCGCGTTCGTCTCCTACGGACAGCAGCAGTGGGC |
| | CACCACCACCGCCTACCCCGGCGCCGACCGCACCGACGTCACCCCGCCCAACGGC |
| | AAATACCCGACCAGCACCTTCACCGACGGCCGCAACCAGGTCAGCGCCCTGTGGC |
| | AGTACCGCACCGCCACCCCCACCGGCAACCCGGCCGACGCGACCGTCACCACCTA |
| | CACCTACGACGCCGCCAACCGGCCCGCCACCCGCAAGGACGCCGCCGGGAACACC |
| | TGGAGCTACGGCTACGACCTGCGCGGCCGCCAGACCACCGTCACCGACCCCGACA |
| | CCGGCACCACCACCACCGCCTACGACGTCAACTCGCGCGCCGTCTCCACCACCGAC |
| | GGCAAGGGCAACACCCTCGTCGTCAGCTACGACCTGATCGGCCGCAAGACCGGCC |
| | TCTACCAGGGCAGCATCGCCCCGGCCAACCAGCTCGCCGGCTGGACGTACGACAC |
| | CCTGCCGGGCGGAAAGGGCAAGCCCACCTCCTCCACCCGCTACGTCGGGGGCGCC |
| | GGCGGCTCGGCCTACACCCAGGCCGTCACCGGCTACGACGCCGGCTACCGGCCCA |
| | CCGGCACCTCGGTGACGATCCCCGCCAGCGAAGGCAAGCTCGCCGGTACCTACAC |
| | CACCGGCCTGACGTACAACCCGGTCCTCGGCACGCTCAAGCAGACCGACCTGCCG |
| | GCCATCGGCGCGGCGCCCGCCGAGAGCGTCATGTACACCTACAACATCTCCGGCG |
| | TCCTGCAGAAGTCCTACAGCGACACCTACTACGTCTACGACGTGCAGTACGACGCC |
| | TTCGGCCGCCCGGTCCGCACGACCACCGGCGACGCCGGAACCCAGGTCGTCTCCA |
| | CCCAGCTCGACAAGACCGACTACACCTACAACCAGGCCGGCGACGTCACCTCGGT |
| | CACCGACGTCCAGAACGGCACCGCCACCGACGCCCAGTGCTTCACCTACGACCAC |
| | CTCGGGCGCCTCACCCAGGCCTGGACCGACACCGCGGGCTCCACCAGCACCACCA |
| | GCGGCACCTGGACCGACACCTCCGGCACCGTCCACAACAGCGGCTCCTCCCAGTC |
| | CGTCCCCGCACTCGGCGCCTGCGCCAACGCCAACGGCCCCGCCAGCACCGGCAGC |
| | CCCGCCAAGCTCTCCGTCGGCGGCCCCTCCCCGTACTGGCAGAGCTACGGCTACGA |
| | CAGCACCGGCAACCGCACCACCCTCGTCCAGCACGACACCACCGGCAACACCACC |
| | AAGGACACCACCACCACCCAGACCTTCGGCCCCGCCGGATCGGTCAACACCGCCA |
| | CCGGCGCCCCCAACACCGGCGGCGGCACCGGCGGCCCGCACGCCCTGCTCACCAG |
| | CAGCACCACCGGACCCACCGGGACCCAGGTCACCAGCTACCAGTACGACCAGCTC |
| | GGCAACACCACCGCGGTCACCGAGACGTCCGGAACCACCACCCTCGCCTGGAACG |
| | GCGAGGACAAGCTCGCCTCCGTCACCAAGACCGGCCAGGCCCAGGCCACCAGCTA |
| | CCTCTACGACGCCGACGGCAACCAGCTCATCCGCCGCAACCCCGGCAAGACCACC |
| | CTCAACCTCGGCAGCGACGAGGTCACCCTCGACACCGCCGCCAACTCCCTCACCG |
| | ACACCCGCTACTACAGCGCCCCCGGCGGCATCAGCATCGCCCGCACCACCGGACC |
| | CACCGGCGCAAGCGCCCTCGCCTACCAGGCCTCCGACCCCCACGGCACCGCCAAC |
| | GTCCAGATCAACGTCGACGCCGCCCAGACCACCACCCGCCGCCCCACCGACCCCTT |
| | CGGCAACCCCCGCGGCACCCAGCCCGCCCCCAACACCTGGGCCGGCGACAAGGGC |
| | TTCGTCGGCGGCACCAAGGACGACACCACCGGACTCACCAACCTCGGCGCCCGCG |
| | AATACCAACCCACCACCGGCCGCTTCCTCAACCCCGACCCACTCCTCGACGCCGGC |
| | AACCCCCAGCAGTGGAACGGCTACGCCTACAGCGACAACGACCCCGTCAACAGCT |
| | CCGACCCCAGCGGACTCATCACCAACGCCCTGGCCGACGGCGACACCTACGTCGC |
| | CCGCCCCGCCGCCTTCTGCGTCACCATGTCGTGCGTCGAGCAGACCAGCGGCCCCG |
| | GTTTCTGGGAGGACAAGCGCGTCGGTGACGCCGTCTTCGCCGCCGTCGTCCAGGCC |
| | ACCACGCAGAGCAACGGCAACGGGTCATCCCAGACCAAGAAAGAGAAGGGCATC |
| | TGGGGCCAGGCCTGGGACTGGACCAAGAAGAACGGCGGCGCCATCCTCGGAGCGC |
| | TGGTAGAGGGAGCGGTCTTCAGCACATGCTTCATCGGAGCTGGATTCGCCGCACCT |
| | GCAACGGGAGGAATCACCGTCATCGCCGGTGCTGCGGCCTGCGGGGCTGTGGCCG |
| | GCGAGGCAGGGGCACTGACCACCAATATCCTCACCCCAGATGCCGACCACTCCGT |
| | CGACGGCATCACCAACGACATGGTCGTTGGTGAAATCACCGGGGCGGCTGTCAGC |
| | GCAGCGAGCGAGGGCGCAAGCTCCCTCGCCAAGCCGGCGGTCCGCAAACTCCTGG |
| | GCATGGAAGCCGAGGAAGGACTCGAGGCAGCAGGCCGCGCCGCCACCGGACCTT |
| | GCAACAGTTTCCCGGCCGGCGTCACCGTCCTCCTCGCCGACGGCACCACCAAGCCC |
| | ATCGAACAGATCGCCCAGGGCGACCAGGTAACCGCCACCGACCCGCAGACAGGCA |
| | CCACCCAGGCAGAACCCGTCACCGACACGATCATCGGCCACGACGACACGGAATT |
| | CACCGACCTCACCCTCACCAACGACGCAGACCCCCGCGCCCCGCCCAGCGAGATC |
| | ACCTCCACCACCCACCACCCCTACTGGAACGCCACCACCAGCCGCTGGACCGATG |
| | CCGGCGACCTCAAGCCCGGCGACCACGTCCGCACCCCCGACGGCACCGAACTGAC |
| | CGTCAACACCGTCTACAGCTACACCACACAACCCCGGACCGCGCGCAACCTCACC |
| | GTCGCAGACCTCCACACGTACTATGTGCTCGCTGGAAATACGCCGGTCCTAGTGCA |
| | TAACACCGGCCCGGGATGTGGTGAGCCGGGATTCGTTAGTGACGCTGCTAATTCTC |
| | TCTCGGGCAGGCGCATCACCACGGGACAAATATTTGATGCGAGCGGGAATCCGAT |
| | CGGGCCTGAGATCACGAGCGGCGGCGGCAGTCTGGCAGATAGGGCGCAGAGTTAT |
| | CTTGCCGACTCCCCTAATATTCGAAATCTGCCCGCTAAGGCGAGATATGCGTCGGC |
| | TGACCACGTTGAGGCGCAATATGCAGTGTGGATGCGAGAAAATGGAGTGACCGAC |
| | GCCAGTGTGGTCATCAATCAAAACTATGTATGTGGGCTGCCCCTAGGCTGCCAGGC |
| | GGCGGTGCCCGCTATCCTCCCTCGCGGCTCGACCATGACGGTATGGTATCCAGGGT |
| | CAGGAAGTCCCATCGTATTGCGGGGAGTGGGTTAA [SEQ ID NO: 139] |
| DddA homolog in | >ATE59819.1 type IV secretion protein Rhs [Thauera sp. K11] |
| Thauera sp. K11 | MRAFRLIACLLAFSAAAAPAAADTSSMLGRLPEASARQLKERLAPRGLASAAALRQY |
| PROTEIN | LDASQRELDTAPEADDVPARSQRFAARAGELTALREQARRDLASLEDAAKASGSAEA |
| | TQRIGRIRGQVDARFDRLEGLFTTWRNAPQGSERRQARRELRAALATLRHAGTPAPAA |
| | IPVPTLGPLQPAGEPAANPPAARLPAYAQADDATGDPFTPGGFRLMKVAALPPAVAAE |
| | AATDCSATSADLADDGKDVRLTQPIRDLAASLDYSPARILRWTQQNVAFEPYWGALK |
| | GAEGVLQTRAGNSTDQASLLIALLRASNIPARYVRGTVQLNDTAAQDDAGGRAQRW |
| | LGTKRYRASAAVLAGGGTSAGLQSIDGTVRGIRFSHVWVQACVPHGAYRGARAEAG |
| | GYRWLALDAAVKDHDYQQGIAVDVPLTDAAFYTPYLAARSDQLPHEHFAQKVAEAA |
| | RATDANAALADVPYAGTPRPLRYDVLPGSLPYEVEAFTNWPGLGSSETASLPDAHRH |
| | TFTVTVRNGATTLASAALPYPQNAFKRVTLSYQPTAASQAAWNAWTGDLPAAADGSI |
| | QVVPQIKADGTVLAAGAPANALPLAGVHNVILKVSQGERSGAACINDSGNPADPKDT |
| | DGTCLNKTVYTNIKAGAYHALGLNALHTSNAFLGQRLEALAAGVQAYPVAPTPAAG |
| | AGYEATVGELLHLVLQDYLHQTEQADQRNAALRGFKSVGPYDLGLTASDLETDYLFD |
| | IPVAIKPAGVFVDFKGGLYGFVKLDTTAETAAARAAENVDLAKLSIYSGSALEHHVW |
| | QQALRTDAVSTVRGLQFAAEQGIPLVTFTAANIGQYDSLMQMSGATSMAAYKSAIQN |
| | AVKGSDNGNHGVVTVPRAQIAYADPVDPASKWTGAVYMSQNPVTGEYGAIINGTIAG |
| | GFPLLNSTPFSNLYNFDSFVPNTLLGTNGGAGAVQTLPGGTQGESSWITKAGDPVNML |
| | TGNYTLQARDFTIKGRGGLPIVLERWFNAQNATDGPFGFGWTHSFNHQLRFYGIESGQ |
| | SKVGWVDGTGAQRFYAVAAAGSIAPGTTLAAQAGVFTTLSRLADGRFQVRETNGLT |
| | YSFESLTSPTTPPAAGSEPRARLLAIADRHGNTLTLNYSGSQLASVSDSLGRTVLSFTW |
| | NGNRIGKVKDVSGREVNYAYEDGNGNLTRVTDPLGQATRYSYYTSADGAKLDHALR |
| | RHTLPRGNGMEFEYYAGGQVFRHTPFDTSGNLIPESALTFHYNSYRRESWTVDGRGA |
| | EERFLFDTHGNVIQQTAANGATHTYAYADPNDPHLRTRMTDPVGRVTQYSYTAEGYL |
| | QTLTLPSGAVQAWRDYDAFGQPRRVKDARGNWTLHHYDTAGTRTDSIRVKSGVVPT |
| | VGTAPAAANVVSWIKYQGDSVGNLTGVKRLRDWTGATLGNFASGSGPVVTTTFDAA |
| | RLNVASVGRSGNRNGSQISETSPIFSHDALGRLTGGVDGRWYPVAFDYDVLDRVTRA |
| | TDATGQPRRYAFDVNGNRIGTELIAGGSRIDSSVAAFDVQDRVAHVLDHAGNRVAYA |
| | YDAVGNRVSVESPDGYAIGFDYDLAGRPYSAYDEDGNRVFSAFDVAGRVRAVIDPNG |
| | AATLYDYHGDEQDGRLARVEQPAIPGQNAGRAAETDYDAGGLPIRVRQVSAGGEAR |
| | EGYRFHDELGRVVRSVSAPDDVGQRLQVCYSYDALSNLTQVRAGATTDTTSAACAGS |
| | PAVQLTQSWDDFGNLLTRTDALGRVWKFEYDAHGNLVASQTPEQAKVSTRSTYRYD |
| | PALHGLLAGRSVPGSGSAGQSVSYARNALGQVIRAETRDGAGNLVVAYDYQYDAAH |
| | RVVRIVDSRGGKALDYAWTPAGRLASITLDGHVWRFQYDGVGRLAAIVAPNGATIA |
| | MARDAAGRLTERRWPDGAKSAFDWLPEGSLAAIEHSAGGSALAQFAYAYDAWGNR |
| | TSATETLAGTSRSLAYGYDALDRLKTVTTDGATETHAFDLFGNRTSKTTGGVTTDYLF |
| | DAAHQLTQVQIAGTPTERLAYDDNGNLRKHCVGSPSGSTSDCTGTTVLSLAWNGLDQ |
| | LIQAARTGLPAESYAYDDAGRRVTKAVGSSATHFAYDGPDILAEYASPAGSPTAVYA |
| | HGAGIDEPLLRLTGATSTPAASAHHYAQDGLGSIVAAYGEIGASGPVSAASVSATHSY |
| | SAGSYPPAKLIDGETTGSTGFWAGSSGNFAADPAVITLELGAEKSVSRVRLHRVASYLP |
| | DYVVKDAEVQVRKPDNSWQTVGTLTNNTSEDSPEIVLTGAPGSALRVLVKGVRNGSL |
| | VLMAEVTMSADGGAASVATARYDAWGNVTQASGSIPAFGYTGREPDATGLVYYRAR |
| | YYHPALGRFASRDPLGLAAGINPYAYAGGNPILYNDPDGLLAQLAWNTAASYWGQPI |
| | VQETVATIRNGAAVAAGNFVPDTVNGATGWFEQFLHQESGSFGRMDSWVDVRNPVA |
| | QDVAQDLRGVAAVGLMMTPLRYGRASNASFNPPVANLPLNTGGKTSGMLHIPGQESL |
| | SLTSGIAGPSQVVRGQGLPGFNGNQLTHVEGHAAAYMRTHKVSEAVLDINKAPCTAG |
| | SGGGCNGLLPRMLPEGAHLTIRHPNGVQVYIGTPD [SEQ ID NO: 140] |
| DddA homolog in | >CCZ27_07525 NZ_CP023439.1:1708666-1716450 Thauera sp. K11 chromosome, |
| Thauera sp. K11 | complete genome |
| DNA | ATGCGTGCCTTCCGCCTGATCGCCTGCCTTCTCGCCTTTTCGGCGGCAGCCGCACCT |
| | GCTGCGGCTGACACGTCGTCGATGCTGGGGCGTCTGCCTGAAGCAAGCGCCCGCC |
| | AGCTCAAGGAGCGGTTGGCGCCGCGTGGCCTTGCCTCCGCTGCCGCCTTGCGCCAG |
| | TACCTGGACGCCTCGCAACGCGAGCTGGACACCGCACCGGAAGCGGACGACGTAC |
| | CCGCCCGCAGCCAACGCTTTGCCGCAAGGGCGGGCGAACTCACCGCGCTGCGCGA |
| | ACAGGCGCGCCGGGATCTCGCCAGTCTGGAGGACGCCGCGAAGGCGAGCGGCTCG |
| | GCCGAGGCGACGCAGCGCATCGGTCGAATCCGCGGGCAGGTGGACGCACGCTTCG |
| | ACCGGCTCGAAGGGCTTTTTACCACTTGGCGCAATGCGCCCCAGGGCAGCGAACG |
| | CCGCCAGGCCCGCCGCGAACTGCGTGCCGCGCTCGCCACGCTCCGCCATGCCGGC |
| | ACCCCGGCTCCGGCTGCGATTCCTGTTCCTACCCTCGGCCCCCTGCAACCGGCCGG |
| | CGAGCCGGCTGCCAACCCACCGGCCGCGCGCTTGCCAGCCTATGCGCAAGCGGAT |
| | GACGCGACTGGCGACCCCTTTACCCCCGGTGGCTTCCGGCTGATGAAGGTCGCCGC |
| | ACTGCCGCCGGCGGTCGCGGCCGAGGCGGCAACGGACTGCTCCGCCACCAGCGCC |
| | GACCTGGCCGACGACGGCAAGGACGTGCGCCTGACCCAGCCGATCCGCGACCTCG |
| | CGGCATCGCTCGACTACTCACCGGCACGCATCCTGCGCTGGACGCAGCAGAACGT |
| | CGCCTTCGAACCCTACTGGGGGGCACTCAAGGGGGCGGAAGGCGTGCTGCAGACG |
| | CGCGCCGGCAACAGCACCGACCAGGCCAGCCTGCTGATCGCACTCTTGCGGGCCT |
| | CCAACATTCCCGCCCGCTACGTACGCGGCACCGTGCAGCTCAACGACACTGCCGC |
| | GCAGGACGACGCAGGCGGGCGGGCGCAGCGCTGGCTGGGCACCAAGCGCTACCG |
| | TGCATCGGCCGCGGTACTCGCCGGCGGCGGAACTTCCGCCGGCCTGCAGTCGATC |
| | GACGGCACCGTCCGCGGCATCCGCTTCAGCCATGTCTGGGTCCAGGCCTGCGTTCC |
| | CCATGGCGCTTACCGCGGTGCCCGCGCGGAAGCCGGCGGCTATCGCTGGCTGGCG |
| | CTGGACGCGGCGGTGAAGGACCATGACTACCAGCAGGGCATCGCGGTCGATGTGC |
| | CGCTCACCGATGCCGCGTTCTACACGCCCTATCTGGCGGCGCGCAGCGACCAGTTG |
| | CCGCACGAGCATTTCGCACAGAAGGTGGCGGAGGCGGCGCGTGCGACCGACGCCA |
| | ATGCGGCGCTGGCCGACGTGCCCTACGCCGGTACGCCGCGGCCGCTGCGCTACGA |
| | CGTGCTGCCCGGTTCGCTGCCCTACGAGGTCGAAGCCTTCACCAACTGGCCCGGCC |
| | TCGGTTCGTCCGAAACCGCAAGCCTGCCGGACGCACACCGCCACACCTTCACCGTG |
| | ACGGTCAGGAACGGCGCCACCACGTTGGCGAGCGCCGCGCTGCCCTATCCGCAGA |
| | ACGCCTTCAAGCGCGTCACGCTGTCCTATCAGCCGACTGCCGCCTCGCAGGCGGCC |
| | TGGAACGCCTGGACGGGCGATCTGCCCGCCGCGGCCGACGGCAGCATCCAGGTCG |
| | TGCCGCAGATCAAGGCCGACGGTACCGTGCTCGCCGCAGGTGCGCCCGCCAACGC |
| | GCTGCCGCTCGCCGGCGTGCACAACGTCATCCTCAAGGTCTCGCAGGGCGAGCGC |
| | AGCGGTGCCGCGTGCATCAACGACAGCGGCAACCCCGCCGACCCGAAGGACACCG |
| | ACGGCACCTGCCTCAACAAGACCGTCTACACCAACATCAAGGCCGGCGCCTACCA |
| | CGCCCTGGGCCTGAATGCGCTGCACACCTCGAATGCCTTCCTCGGCCAGCGGCTCG |
| | AAGCGCTGGCGGCCGGCGTGCAGGCCTATCCCGTCGCGCCCACGCCGGCCGCGGG |
| | TGCCGGCTACGAGGCCACGGTCGGTGAATTGCTGCATCTGGTGCTGCAGGACTACC |
| | TGCACCAGACCGAGCAGGCCGACCAGCGCAACGCCGCGTTGCGCGGCTTCAAGAG |
| | CGTGGGGCCGTACGACCTCGGGCTGACCGCGTCCGACCTCGAAACCGACTACCTCT |
| | TCGACATCCCGGTCGCGATCAAGCCGGCCGGCGTGTTCGTGGACTTCAAGGGCGG |
| | CCTCTACGGTTTCGTCAAACTCGATACCACGGCCGAGACGGCCGCGGCACGCGCC |
| | GCCGAAAACGTGGATCTGGCCAAGCTCTCGATCTACTCCGGCTCCGCGCTCGAACA |
| | CCACGTCTGGCAGCAGGCGCTGCGCACCGATGCGGTGTCCACCGTGCGTGGGCTG |
| | CAGTTCGCCGCCGAGCAGGGCATTCCGCTCGTCACCTTCACCGCGGCCAACATCGG |
| | CCAGTACGACAGCCTCATGCAGATGAGCGGCGCCACCAGCATGGCCGCTTACAAG |
| | AGCGCGATCCAGAACGCGGTGAAGGGCTCGGACAACGGCAACCACGGCGTCGTCA |
| | CCGTGCCGCGCGCCCAGATCGCCTACGCCGACCCCGTCGATCCGGCGAGCAAATG |
| | GACCGGCGCGGTCTACATGTCTCAGAACCCCGTCACCGGAGAGTACGGGGCGATC |
| | ATCAACGGCACCATCGCCGGCGGCTTCCCGCTGCTCAACAGCACGCCCTTCAGCAA |
| | TCTCTACAACTTCGATTCCTTCGTGCCCAACACCCTCCTTGGCACGAACGGGGGTG |
| | CCGGTGCGGTGCAGACCCTGCCCGGCGGCACCCAGGGCGAGAGTTCCTGGATCAC |
| | CAAGGCCGGCGACCCGGTGAACATGCTCACCGGCAACTACACGCTGCAGGCACGC |
| | GACTTCACCATCAAGGGCCGGGGCGGACTGCCGATCGTGCTGGAGCGCTGGTTCA |
| | ACGCGCAGAACGCCACCGACGGGCCGTTCGGCTTCGGCTGGACGCACAGCTTCAA |
| | CCATCAGTTGCGTTTCTACGGCATCGAGAGCGGCCAGTCCAAGGTCGGCTGGGTG |
| | GACGGCACTGGCGCCCAGCGCTTCTACGCCGTGGCCGCCGCCGGCAGCATTGCGC |
| | CGGGCACGACGCTGGCCGCGCAGGCCGGGGTGTTCACGACGCTGTCGCGTCTGGC |
| | CGACGGCCGCTTCCAGGTGCGCGAGACCAACGGCCTCACCTACAGCTTCGAATCG |
| | CTCACGAGCCCGACCACCCCGCCGGCCGCGGGCAGCGAACCGCGCGCAAGACTGC |
| | TGGCCATCGCCGACCGCCACGGCAACACCCTGACGCTCAACTACAGCGGCAGCCA |
| | GCTTGCCTCGGTGAGCGACAGCCTCGGCCGCACGGTGCTCAGCTTCACCTGGAACG |
| | GCAACCGCATCGGCAAGGTGAAGGACGTCAGCGGACGGGAAGTGAACTACGCCT |
| | ACGAGGACGGCAACGGCAACCTCACGCGCGTCACCGATCCGCTGGGTCAAGCCAC |
| | GCGCTACAGCTACTACACCAGTGCCGACGGTGCCAAGCTCGACCACGCCCTGCGC |
| | CGCCACACCCTGCCGCGCGGCAACGGCATGGAGTTCGAGTACTACGCCGGTGGCC |
| | AGGTCTTCCGCCACACGCCGTTCGACACCAGCGGCAACCTCATTCCCGAATCGGCG |
| | CTGACCTTCCACTACAACAGTTATCGGCGCGAGAGCTGGACGGTCGATGGCCGCG |
| | GTGCCGAGGAGCGCTTCCTGTTCGACACGCACGGCAACGTGATCCAGCAGACCGC |
| | CGCCAACGGTGCCACCCACACCTACGCGTACGCCGACCCGAACGATCCGCATCTG |
| | CGCACGCGCATGACAGACCCGGTCGGCCGCGTCACCCAGTACAGCTATACCGCCG |
| | AAGGCTATCTGCAGACCCTGACGCTGCCGTCGGGCGCCGTGCAGGCGTGGCGCGA |
| | CTACGACGCCTTCGGCCAGCCCCGCCGCGTCAAGGACGCGCGCGGCAACTGGACG |
| | CTCCACCACTACGACACCGCCGGGACACGGACCGACTCCATCCGGGTCAAATCGG |
| | GCGTGGTCCCCACCGTCGGCACCGCGCCTGCCGCGGCCAACGTCGTTTCCTGGATC |
| | AAGTACCAGGGCGACAGCGTGGGCAACCTCACCGGCGTCAAGCGCCTGCGCGACT |
| | GGACGGGCGCGACCCTGGGCAATTTCGCCAGCGGCAGCGGCCCCGTCGTCACCAC |
| | CACCTTCGATGCGGCCAGGCTCAACGTCGCCAGCGTCGGCCGTAGCGGCAACCGC |
| | AACGGCAGCCAGATCAGCGAGACCAGCCCGATCTTCTCCCACGACGCGCTGGGGC |
| | GCCTCACCGGCGGGGTGGACGGGCGCTGGTATCCGGTCGCCTTCGATTACGACGT |
| | GCTCGACCGCGTCACCCGCGCCACCGACGCCACGGGCCAGCCGCGCCGCTACGCG |
| | TTCGACGTCAACGGCAACCGCATCGGTACGGAGCTGATTGCCGGCGGCAGCCGTA |
| | TCGATTCCTCGGTGGCCGCCTTCGACGTGCAGGACCGCGTCGCCCACGTCCTCGAT |
| | CACGCCGGCAACCGCGTGGCCTACGCCTACGATGCGGTGGGCAACCGGGTGAGCG |
| | TGGAAAGCCCCGACGGCTACGCCATCGGCTTCGACTACGACCTCGCCGGACGGCC |
| | CTATTCGGCCTACGACGAAGACGGCAACCGCGTCTTCTCCGCCTTCGACGTGGCCG |
| | GGCGCGTGCGAGCGGTCATCGACCCCAACGGCGCCGCGACGCTCTACGACTATCA |
| | CGGCGACGAGCAGGACGGGCGTCTCGCGCGCGTGGAGCAGCCCGCCATCCCGGGC |
| | CAGAACGCGGGCCGCGCCGCCGAGACCGACTACGATGCGGGTGGGTTGCCCATCC |
| | GCGTGCGCCAGGTCTCGGCCGGCGGCGAAGCGCGCGAAGGCTACCGTTTCCACGA |
| | CGAGCTTGGCCGCGTGGTGCGCAGCGTCTCCGCGCCGGACGACGTCGGCCAGCGG |
| | CTGCAGGTCTGCTACAGCTACGATGCACTCTCGAACCTCACCCAGGTGCGCGCCGG |
| | CGCCACCACCGACACCACCAGTGCCGCCTGCGCCGGCAGCCCCGCGGTGCAGCTC |
| | ACCCAGAGCTGGGACGACTTTGGCAACCTGCTGACGCGCACCGACGCGCTGGGCC |
| | GGGTGTGGAAGTTCGAGTACGACGCCCACGGCAACCTCGTCGCCAGCCAGACGCC |
| | CGAGCAGGCCAAGGTCTCGACGCGCAGCACCTACCGCTACGATCCGGCGCTGCAC |
| | GGCTTGCTGGCCGGGCGCAGCGTGCCGGGCAGCGGCAGTGCGGGCCAGAGCGTGA |
| | GCTATGCGCGCAACGCGCTCGGCCAGGTCATCCGCGCCGAGACGCGCGACGGCGC |
| | GGGCAACCTCGTCGTCGCCTACGACTACCAGTACGACGCCGCCCACCGTGTGGTGC |
| | GCATCGTCGACAGCCGCGGCGGCAAGGCGCTCGACTACGCCTGGACGCCCGCCGG |
| | GCGGCTGGCGAGCATTACCCTGGACGGCCATGTCTGGCGCTTCCAGTACGACGGC |
| | GTCGGCCGGCTCGCCGCGATCGTCGCGCCCAACGGCGCCACCATAGCGATGGCAC |
| | GCGATGCCGCCGGGCGGCTCACCGAGCGGCGCTGGCCCGACGGCGCGAAGAGCGC |
| | CTTCGACTGGCTGCCCGAAGGCAGCCTCGCCGCCATCGAGCACAGCGCGGGCGGC |
| | AGCGCGCTCGCACAGTTCGCCTATGCCTACGATGCCTGGGGCAACCGCACGAGCG |
| | CCACCGAGACCCTCGCGGGCACCAGCCGCAGCCTCGCCTACGGCTACGACGCGCT |
| | CGACCGCCTGAAGACCGTCACCACCGACGGTGCGACCGAAACCCATGCCTTCGAT |
| | CTCTTCGGCAATCGCACCAGCAAGACCACGGGCGGGGTGACCACCGACTATCTCTT |
| | CGACGCGGCGCACCAGCTCACCCAGGTGCAGATCGCCGGCACCCCCACCGAGCGG |
| | CTCGCCTACGACGACAACGGTAATCTCCGCAAGCACTGCGTCGGCAGTCCGAGTG |
| | GCAGCACCAGCGATTGCACCGGCACCACCGTGCTGAGCCTCGCCTGGAACGGCCT |
| | CGACCAGTTGATCCAGGCCGCCAGGACGGGCCTGCCCGCCGAGTCCTACGCCTAC |
| | GACGATGCCGGGCGGCGTGTCACCAAGGCGGTGGGCAGCAGCGCCACCCACTTCG |
| | CCTACGACGGTCCCGACATCCTGGCCGAGTACGCCAGCCCGGCCGGCAGCCCCAC |
| | CGCCGTCTATGCCCACGGTGCCGGCATCGACGAACCGCTGCTGCGCCTCACCGGCG |
| | CGACGAGCACGCCGGCCGCTTCCGCGCACCACTACGCGCAGGACGGGCTGGGCAG |
| | CATCGTCGCGGCCTATGGCGAGATCGGCGCCAGCGGTCCGGTCAGTGCCGCGAGC |
| | GTATCGGCCACCCACAGTTACAGCGCCGGCAGCTACCCGCCGGCAAAGCTGATCG |
| | ACGGCGAGACGACCGGAAGCACCGGGTTCTGGGCTGGCAGCTCGGGCAACTTCGC |
| | TGCCGATCCAGCCGTGATCACGCTGGAACTGGGTGCGGAGAAAAGCGTGAGCCGC |
| | GTGAGGCTGCACCGGGTGGCCAGCTACCTGCCCGACTACGTGGTCAAGGATGCCG |
| | AGGTGCAGGTCCGAAAACCGGACAATTCGTGGCAGACGGTCGGCACGCTGACAAA |
| | CAACACCAGCGAAGACAGTCCCGAGATCGTGCTCACCGGCGCCCCCGGCAGCGCG |
| | CTGCGCGTGCTCGTCAAGGGCGTGCGCAACGGCAGCCTGGTGCTGATGGCCGAGG |
| | TGACGATGAGTGCGGACGGTGGCGCGGCCAGCGTGGCCACCGCCCGCTACGACGC |
| | CTGGGGCAACGTCACGCAGGCGAGCGGCAGCATCCCGGCCTTCGGCTACACCGGA |
| | CGCGAGCCCGATGCCACGGGCCTGGTCTACTACCGCGCCCGCTACTACCACCCCGC |
| | GCTCGGCCGCTTCGCCAGCCGCGACCCGCTGGGGCTGGCGGCGGGGATCAATCCC |
| | TACGCCTACGCGGGCGGCAATCCCATCCTCTACAACGATCCGGATGGCTTGCTGGC |
| | GCAACTGGCGTGGAATACGGCGGCCAGCTACTGGGGACAGCCGATAGTTCAAGAA |
| | ACGGTCGCCACGATTCGAAATGGGGCCGCAGTGGCCGCTGGCAACTTCGTTCCAG |
| | ACACGGTCAACGGTGCAACAGGTTGGTTTGAGCAGTTCCTGCACCAAGAATCGGG |
| | CTCGTTCGGGCGCATGGACTCGTGGGTGGATGTGCGAAACCCCGTTGCGCAGGAC |
| | GTAGCCCAGGACCTGCGCGGTGTCGCAGCCGTTGGGTTAATGATGACGCCGCTGC |
| | GGTATGGTCGTGCCTCCAACGCGTCTTTCAATCCGCCAGTAGCCAATCTTCCGCTC |
| | AACACTGGAGGAAAAACATCTGGCATGTTGCACATTCCAGGGCAAGAATCACTGT |
| | CGCTCACGAGCGGAATTGCGGGGCCGTCTCAAGTCGTTAGAGGTCAAGGTTTGCC |
| | AGGATTCAACGGTAATCAGTTGACCCATGTGGAAGGTCATGCTGCTGCTTACATGC |
| | GGACTCACAAGGTCTCTGAGGCTGTTCTGGACATAAACAAAGCACCTTGCACCGCT |
| | GGTAGTGGTGGTGGATGTAATGGGTTGCTTCCCCGAATGCTGCCGGAGGGGGCTC |
| | ATTTAACAATTCGACACCCAAATGGTGTTCAAGTTTATATTGGCACTCCTGACTAA |
| | [SEQ ID NO: 141] |
| Chondromyces | >AKT41505.1 type IV secretion protein Rhs [Chondromyces crocatus] |
| crocatus | MSMSASRSQPAFPFVSASSPRPRRRPPFPRALLLLIAVLLVGACGDAGGPLLWSSSSQA |
| PROTEIN | LWEPSPIPPLPPLLCLGPGDGPSPFPPDLTQGTTTAAGTLPGSFSVTSTGEATYTIPVPTL |
| | PGRAGIEPSLAITYDSAQGEGLLGIGFHLQGLSSVDRCPRNVAQDGHIAPVRDAEDDAL |
| | CLDGQRLVPVDPQPGRAPREYRTFPDSFTRVEADFAESEGWPAERGPKRLRAHGKAG |
| | LIYEYGGESSGRVLAQGEAVRSWLLTRLSDRDGNTMAVVYRNDLHAKGYTVEHAPQ |
| | RITYTRHPTVPASRMVEFTYGPLEAADVRVHYARGMELRRSLSLRSIQMFGPGHVLAR |
| | ELRFGYGHGPATGRLRLEAVRECAGDGTCKPPTRFTWHTAGAAGYTQQQTLVEVPLS |
| | ERGTLMTMDVSGDGLDDLVTSDMVVEAGTEEPITRWSVALNRSQELTPGFFEAAVTG |
| | QEQPHFIDAEPPYQPELGTPLDYDHDGRMDLFLHDVHGQSMTWEVLLSNGDGRFTRR |
| | DTGVPRPFTMGMTPAGLRSPDASTHLVDVDGDGMVDLLQCYLSAHEQLWYLHRWT |
| | AAAGGFAPHGDRVHALSSYPCHAELHAVDVDADGRVDLVMQELILVGSQVRAGWQ |
| | YVAFSYELSDGSWTRALTGLRLTPPGDRVFFLDVNGDGLPDAVQSSRDDEQLYTSMNI |
| | GAGFAAPVPSLATPTLGAARFVRFASVLDHNADGRQDLLLAMSDGGSESLPAWKVLQ |
| | ATGEVGPGTFEIVHPGLPMGIVLQQDELPTPDHPLTPRVTDVNGDGAQDLLYAFNNQV |
| | HVFENVLGQEDLLAAVTDGMNAHAPEDAEYLPNVQIRYDHLIDRARTTEGFEDAPGIP |
| | SPEQRTYRPLEQSDEEPCRYPVRCVVGHRRVVSGYVLNNGADRPRTFQVAYRNGRHH |
| | RLGRGFLGFGTRIVRDLDTGAGTAEFYDNVTFDGAFQAFPFRGQVQRSWRWSPSLPL |
| | DAHSAEPASLELLTTRSYAVVIPTQAGTYFTLSLLEGKSRHQGTFSPGSGKTLEEAVRA |
| | LEGDLASRMSDTLRTVSDFDLYGNILAEQTQTEGVDLDLSVTRSFDNDPLSWRLGELT |
| | RETTCSKAGGETQCRVMHRSYDGRGHVRLERVGGEPFDPEMQLDVWFSRDALGNIH |
| | STRSRDGTGQVRASCTSYDALGLMPYAHRNLEGHQSYTRYDPAVGVLRASVDPNGL |
| | VSRWAYDGFGRVTLESLPGRMPTVIRRTWTKDGGAAGNAWNLKIRTASVGGQDETV |
| | QLDGLGREVRWWWQALDVGEEQAPRMMQEVAFDARGEHLAWRSLPIVDPAPPGSV |
| | QVRETWQYDGMGRVLRHVTPWGAATTHEYIGRDEVITAPGQAVTRIASDPLGRPTAV |
| | GDPEGGVSRYTYGPFGGLREVTTPAGAVTLTERDAFGRVRRQVSPDRGVSTAHYDGY |
| | GQKISSLDAAGRAVTTRYDTLGRIFRQVDEDGVTEFRWDDAQHGVGQLALVVSPDGH |
| | RLRYGFDHLGRPATTTLEIGGESFTSRLSYDLSGRLERIEYPSAPGIGSFAIEREYDPHGR |
| | LRALKDAGSGAEFWRATAIDAGNRITGERFGGGTATTLRTFDAARERVSRIETQTAGG |
| | PVQQLSYLWNDRRKLVERSDGLHANVERFRYDLLDRLTCAQFGLINAALCERPFTYG |
| | PDGNLLQKPGVGAYEYDPAQPHAVVRAGSAFYGYDAVGNQTSRPGATIAYTAFDLPK |
| | RIALTSGDTVDFAYDGLQQRVRKTTATQEIASFGEVYERVTDVVTGAVEHRYHVRND |
| | ERVVTLVRRSVAQGTRTLHVHVDHLGSIDVLTDGVTGSVAERRSYDAFGAPRHPDWG |
| | SGQPPSPHELSSLGFTGHEADLDLGLVNMKGRIYDPKLGRFLTPDPLVPRPLFGQSWNS |
| | YSYVLNSPLSLVDPSGFQEQPPATEDGCSQGCTIWVFGPPREPKPPAPPKVVEGNLEDA |
| | AGTGSTQAPVDVGTSGVRSGWSPQLPATLQTLGRGDAIARRIMDGVRIGMARMLLES |
| | AKLGILGGTSRVYVAYTNLTAAWNGYKESGLPGALDAVNPASQMVQAGVEAYEAA |
| | AAEDWEAAGASLFKAGSIGMSILATAVGVGGAITATVGSTAGAAGRAAARAPSLPAY |
| | AGGKTSGVLRTTAGDTALLSGYKGPSASMPRGTPGMNGRIKSHVEAHAAAVMREQG |
| | MKEGTLYINRVPCSGATGCDAMLPRMLPPDAHLRVVGPNGYDQVFVGLPD [SEQ ID |
| | NO: 142] |
| Chondromyces | >CMC5_057130 NZ_CP012159.1:7808731-7815414 Chondromyces crocatus |
| crocatus | strain Cm c5, complete genome |
| DNA | ATGTCCATGTCGGCCTCACGGAGTCAGCCCGCATTCCCCTTCGTGTCGGCCTCCTCT |
| | CCGCGTCCGCGCCGGCGCCCTCCCTTTCCCCGAGCGCTGCTCCTCCTCATCGCCGT |
| | GCTCCTCGTCGGCGCATGCGGCGACGCTGGCGGCCCGCTTCTCTGGTCGAGCAGCT |
| | CCCAGGCCCTCTGGGAACCCTCCCCGATCCCGCCGCTCCCCCCGCTCCTGTGCCTC |
| | GGCCCCGGCGACGGTCCCTCCCCCTTTCCGCCTGACCTTACGCAGGGGACCACCAC |
| | CGCGGCGGGGACCCTGCCAGGGAGCTTTTCGGTCACGAGCACGGGCGAGGCGACG |
| | TACACGATCCCGGTCCCCACGCTGCCTGGCCGTGCCGGCATCGAGCCCTCGCTGGC |
| | GATCACCTACGACAGTGCGCAGGGTGAAGGGCTGCTCGGGATCGGCTTCCACTTG |
| | CAGGGCCTCTCGTCGGTCGATCGCTGCCCCCGGAACGTCGCGCAGGATGGTCACAT |
| | CGCGCCGGTCCGGGATGCCGAGGACGACGCCTTGTGCCTCGATGGGCAGCGGCTC |
| | GTCCCCGTGGACCCGCAGCCAGGGCGTGCGCCGCGGGAATACCGCACGTTCCCGG |
| | ACAGCTTCACGCGCGTCGAGGCCGACTTCGCGGAGAGCGAGGGGTGGCCGGCGGA |
| | GCGTGGGCCGAAGCGGCTGCGGGCGCATGGCAAAGCGGGGCTGATCTACGAATAC |
| | GGTGGAGAATCATCGGGCCGGGTGCTCGCGCAAGGGGAGGCGGTGCGGTCCTGGT |
| | TGCTGACGCGGCTCAGCGACCGGGATGGCAACACGATGGCGGTGGTCTACCGGAA |
| | TGACCTCCACGCGAAGGGCTACACCGTCGAGCACGCGCCGCAGCGGATCACCTAC |
| | ACCAGGCACCCGACTGTGCCGGCCTCGCGCATGGTGGAGTTCACGTACGGGCCGC |
| | TGGAGGCGGCGGACGTGCGCGTACACTATGCCCGCGGGATGGAGCTGCGCCGCTC |
| | GCTGAGCTTGCGCTCGATCCAGATGTTCGGGCCGGGACACGTGCTCGCGAGGGAG |
| | CTGCGCTTCGGTTACGGGCATGGGCCGGCGACGGGTCGCTTGCGACTGGAGGCGG |
| | TTCGGGAGTGCGCAGGTGACGGGACGTGCAAGCCGCCGACACGCTTCACCTGGCA |
| | CACGGCCGGAGCGGCTGGATACACGCAGCAGCAGACACTGGTGGAGGTGCCGCTG |
| | TCGGAGCGCGGCACGTTGATGACGATGGACGTCAGCGGCGATGGCCTCGACGACC |
| | TGGTGACGTCCGACATGGTGGTGGAGGCCGGCACGGAAGAGCCGATCACCCGCTG |
| | GTCGGTCGCGCTCAACCGGAGCCAGGAGCTGACGCCGGGGTTCTTCGAGGCGGCC |
| | GTCACTGGGCAGGAGCAGCCGCATTTCATCGACGCAGAGCCGCCGTACCAGCCGG |
| | AGCTGGGGACGCCGCTCGACTACGACCACGATGGCCGGATGGACCTGTTTCTGCA |
| | CGATGTGCACGGGCAGTCGATGACGTGGGAGGTGCTGCTGTCGAATGGAGATGGG |
| | CGGTTCACGCGGCGGGATACGGGGGTGCCGCGGCCGTTCACGATGGGCATGACGC |
| | CGGCGGGATTGCGCAGCCCGGATGCGTCGACCCATCTGGTGGATGTTGACGGTGA |
| | CGGGATGGTGGACCTGCTGCAGTGCTACCTGAGCGCGCACGAGCAGCTCTGGTAC |
| | TTGCACCGCTGGACGGCAGCGGCGGGGGGCTTCGCGCCGCACGGCGATCGGGTGC |
| | ATGCGCTGAGCTCCTACCCGTGCCACGCCGAGCTGCACGCGGTCGATGTCGACGC |
| | GGATGGGCGGGTGGACCTGGTGATGCAGGAGCTGATCCTCGTCGGGAGCCAGGTG |
| | CGGGCGGGGTGGCAGTACGTGGCGTTCTCGTACGAGCTGTCCGATGGATCGTGGA |
| | CGCGCGCGCTGACGGGGCTGCGGCTCACGCCGCCTGGGGACCGGGTGTTCTTCCTC |
| | GACGTCAACGGCGATGGGCTGCCCGATGCGGTGCAGAGCAGCCGGGACGATGAGC |
| | AGCTGTACACGTCGATGAATATCGGCGCGGGATTCGCGGCGCCGGTACCGAGCCT |
| | GGCGACGCCGACGCTCGGGGCTGCGAGGTTCGTTCGGTTTGCGTCGGTGCTCGATC |
| | ACAACGCGGATGGGCGACAAGACCTGCTGCTGGCCATGAGCGATGGGGGATCGGA |
| | GTCGCTGCCCGCGTGGAAGGTGCTCCAGGCGACGGGGGAGGTCGGTCCGGGGACG |
| | TTCGAGATCGTCCATCCCGGGCTGCCGATGGGCATCGTGCTCCAGCAGGACGAGCT |
| | GCCCACGCCCGACCATCCGCTCACGCCGCGGGTCACTGACGTGAATGGGGATGGG |
| | GCGCAGGATCTGCTCTATGCGTTCAACAACCAGGTCCATGTGTTCGAGAACGTGCT |
| | CGGCCAGGAGGACCTGCTCGCGGCCGTGACCGACGGCATGAATGCGCACGCTCCG |
| | GAGGACGCCGAGTACCTGCCCAACGTGCAGATCCGGTACGACCACCTGATCGATC |
| | GTGCGCGGACGACGGAGGGCTTCGAGGATGCTCCAGGGATCCCGTCACCCGAGCA |
| | GCGCACCTACCGGCCTCTGGAGCAAAGCGATGAGGAGCCCTGCCGCTATCCGGTG |
| | CGGTGCGTGGTCGGGCATCGGCGGGTGGTGAGCGGCTATGTGCTCAACAATGGCG |
| | CGGATCGGCCGCGCACCTTCCAGGTGGCCTACCGCAATGGCCGTCACCATCGCCTG |
| | GGCCGAGGGTTTCTGGGGTTCGGGACGCGGATCGTGCGTGACCTCGATACCGGCG |
| | CGGGGACGGCCGAGTTCTACGACAACGTCACGTTTGATGGCGCCTTCCAGGCCTTC |
| | CCTTTCCGAGGGCAGGTACAGCGCTCGTGGCGCTGGAGTCCGAGCTTGCCGCTGG |
| | ACGCGCATAGCGCGGAGCCGGCGTCCCTCGAGCTGCTGACGACGCGGAGCTACGC |
| | GGTGGTGATCCCCACGCAAGCGGGGACGTACTTCACCCTCTCGCTGCTGGAGGGC |
| | AAGAGCCGTCATCAGGGCACGTTCTCACCGGGGAGTGGGAAAACGCTCGAAGAAG |
| | CCGTGCGCGCTCTGGAAGGAGATCTCGCCTCGCGAATGAGCGACACGCTCCGCAC |
| | CGTCAGCGACTTCGACCTCTACGGGAACATCCTCGCCGAGCAAACGCAGACGGAG |
| | GGCGTCGACCTCGACCTCTCGGTGACGCGCAGCTTCGACAACGACCCGCTCTCCTG |
| | GCGCCTTGGCGAGCTGACGCGAGAGACGACGTGCAGCAAAGCGGGCGGTGAGAC |
| | GCAGTGCCGGGTGATGCACCGGAGCTATGACGGGCGCGGCCACGTTCGCCTGGAG |
| | CGCGTCGGGGGAGAGCCCTTCGACCCGGAGATGCAGCTCGATGTCTGGTTCTCGC |
| | GGGACGCGCTGGGCAACATCCACAGCACCCGGTCACGTGATGGGACGGGGCAGGT |
| | GCGCGCGAGCTGCACCAGCTACGACGCGCTGGGCTTGATGCCTTATGCCCACCGC |
| | AACCTGGAGGGCCACCAGAGCTATACGCGCTACGACCCGGCCGTGGGCGTGCTGC |
| | GGGCGTCGGTGGATCCCAACGGCCTGGTGAGCCGCTGGGCCTACGATGGCTTCGG |
| | GCGGGTGACGCTGGAGAGCCTCCCCGGGCGCATGCCCACCGTCATCCGGCGGACC |
| | TGGACGAAGGACGGCGGAGCGGCTGGCAACGCCTGGAACCTGAAGATCCGCACC |
| | GCCTCGGTGGGGGGCCAGGACGAGACCGTGCAGCTCGATGGTCTCGGGCGGGAGG |
| | TGCGCTGGTGGTGGCAAGCGCTCGACGTGGGGGAAGAGCAAGCGCCGCGGATGAT |
| | GCAGGAGGTCGCCTTCGATGCGCGGGGCGAGCACCTCGCGTGGCGCTCGCTGCCG |
| | ATCGTGGATCCCGCGCCACCAGGCTCGGTGCAGGTGCGAGAGACGTGGCAATACG |
| | ACGGGATGGGGGGGGTGCTCCGGCACGTCACGCCGTGGGGGGCGGCGACGACGC |
| | ACGAGTACATCGGGCGGGACGAGGTCATCACCGCGCCTGGGCAGGCCGTCACCCG |
| | AATCGCCAGCGATCCGCTCGGGAGGCCCACGGCAGTGGGTGATCCCGAAGGTGGC |
| | GTCAGCCGGTACACCTACGGTCCCTTCGGGGGGCTGCGCGAGGTGACCACGCCCG |
| | CTGGTGCCGTGACGCTGACCGAGCGGGATGCGTTTGGCCGCGTGCGACGGCAGGT |
| | GAGCCCGGACCGGGGAGTCTCTACTGCGCACTACGACGGTTACGGGCAGAAGATC |
| | TCATCGCTCGACGCGGCAGGACGCGCGGTCACGACCCGCTACGACACGCTGGGTC |
| | GGATTTTCAGGCAGGTCGACGAAGACGGCGTCACCGAGTTCCGTTGGGATGACGC |
| | GCAGCATGGAGTGGGTCAGCTCGCGCTGGTGGTCAGCCCCGATGGGCATCGGCTG |
| | CGCTACGGCTTCGACCACCTCGGGCGACCAGCGACGACGACGCTGGAGATCGGAG |
| | GGGAAAGCTTCACCAGCCGGCTGTCTTATGATCTGAGCGGCCGGCTCGAGCGGAT |
| | CGAGTACCCGAGCGCGCCGGGGATTGGCAGCTTCGCCATCGAGCGGGAGTACGAT |
| | CCTCACGGGCGGCTGCGGGCGCTGAAGGATGCGGGGTCGGGGGCGGAGTTCTGGC |
| | GAGCCACCGCGATCGATGCGGGGAATCGCATCACGGGGGAGCGCTTCGGTGGGGG |
| | GACCGCCACCACGCTCCGCACGTTCGACGCGGCACGGGAGCGGGTGAGTCGGATC |
| | GAGACGCAGACGGCAGGTGGGCCCGTCCAGCAGCTCTCCTACCTCTGGAACGATC |
| | GCCGCAAGCTCGTCGAGCGCTCCGATGGCCTCCACGCCAACGTCGAGCGCTTTCGT |
| | TACGACCTGCTGGACCGGCTGACGTGCGCGCAGTTCGGGCTGATCAATGCTGCCCT |
| | CTGCGAGCGACCGTTCACCTACGGACCCGACGGCAACCTGCTCCAGAAGCCCGGC |
| | GTCGGTGCCTACGAGTACGACCCCGCGCAGCCCCACGCCGTCGTCCGAGCTGGTA |
| | GCGCGTTCTACGGCTACGACGCCGTCGGCAACCAGACCTCACGACCCGGCGCGAC |
| | CATCGCCTACACCGCGTTCGACCTACCGAAGCGAATCGCGCTCACCAGCGGCGAC |
| | ACCGTCGACTTCGCGTACGACGGCCTCCAGCAGCGGGTGCGCAAGACCACGGCGA |
| | CGCAGGAGATCGCCTCCTTCGGCGAGGTGTACGAGCGCGTGACCGATGTCGTCAC |
| | GGGAGCCGTCGAGCATCGCTACCACGTGCGCAACGACGAGCGCGTCGTCACGCTG |
| | GTGCGGCGCTCGGTCGCGCAAGGCACGCGCACGCTGCATGTCCATGTCGACCACC |
| | TCGGGTCGATCGATGTGCTCACCGACGGTGTGACCGGCAGCGTCGCCGAGCGCCG |
| | CAGCTACGATGCCTTCGGCGCACCGCGCCATCCCGACTGGGGTTCGGGTCAGCCTC |
| | CGTCACCCCACGAGCTGTCGTCGCTTGGCTTCACCGGGCACGAGGCCGACCTCGAC |
| | CTCGGCCTCGTGAACATGAAGGGGCGCATCTACGACCCCAAGCTCGGACGGTTCC |
| | TCACGCCCGATCCGCTCGTGCCGCGGCCTCTCTTCGGGCAGAGCTGGAATAGCTAT |
| | TCGTACGTGCTAAACAGCCCGCTGTCGCTGGTCGATCCCAGTGGGTTTCAAGAGCA |
| | GCCACCTGCGACAGAGGACGGATGCTCGCAGGGCTGCACCATCTGGGTGTTCGGT |
| | CCTCCCCGCGAGCCGAAGCCACCTGCGCCGCCCAAGGTCGTCGAGGGCAACCTGG |
| | AGGACGCCGCTGGCACTGGTTCGACCCAGGCGCCGGTCGATGTCGGGACCTCCGG |
| | GGTCCGTAGCGGATGGAGTCCGCAGCTCCCGGCCACGTTGCAGACCTTGGGCCGT |
| | GGTGACGCCATCGCCAGGCGCATCATGGACGGCGTCCGCATCGGGATGGCCAGGA |
| | TGCTGCTGGAGTCCGCAAAGCTCGGCATCCTGGGCGGCACCAGCCGCGTCTACGTC |
| | GCCTACACCAACCTCACCGCCGCCTGGAATGGCTACAAAGAGAGCGGGCTCCCCG |
| | GCGCTCTCGACGCCGTCAATCCCGCCAGCCAGATGGTCCAAGCCGGCGTGGAGGC |
| | CTACGAGGCTGCCGCCGCAGAGGACTGGGAGGCCGCCGGCGCCAGCTTGTTCAAG |
| | GCCGGGTCGATCGGGATGTCGATCCTGGCGACGGCTGTTGGCGTCGGGGGAGCGA |
| | TCACTGCGACAGTGGGCTCGACGGCAGGAGCGGCGGGGAGGGCAGCCGCAAGAG |
| | CCCCCTCACTCCCTGCATATGCTGGCGGAAAAACGTCGGGAGTACTACGGACCAC |
| | CGCAGGCGATACAGCACTGCTGAGCGGCTACAAGGGGCCGTCCGCATCGATGCCT |
| | CGAGGAACGCCAGGCATGAACGGACGCATCAAGTCGCATGTAGAAGCTCATGCGG |
| | CTGCCGTGATGCGAGAGCAAGGGATGAAGGAAGGAACCCTGTACATCAATCGAGT |
| | CCCCTGCTCTGGCGCCACCGGATGCGACGCGATGCTCCCAAGAATGCTCCCACCAG |
| | ATGCACACCTTCGCGTGGTCGGTCCGAATGGTTACGATCAAGTTTTTGTCGGGCTG |
| | CCCGACTGA [SEQ ID NO: 143] |

In addition, the disclosure contemplates the use of any variant derived from any starting point DddA amino acid sequence, for example, a PACE-evolved variant of DddA of SEQ ID NO: 25 (corresponding to residues 1290-1427 of canonical DddA):

| | |
|---|---|
| DddA (residues | GSYALGPYQISAPQLPAYNGQTVGTFYYVNDAGGL |
| 1290-1427) or | ESKVFSSGGPTPYPNYANAGHVEGQSALFMRDNGI |
| (DddAtox) | SEGLVFHNNPEGTCGFCVNMTETLLPENAKMTVVP |
| | PEGAIPVKRGATGETKVFTGNSNSPKSPTKGGC |
| | (SEQ ID NO: 25) |

Exemplary variant DddA fragments derived (e.g., using continuous evolution, such as PANCE or PACE) from SEQ ID NO: 25 can include, for example:

| Mutation(s) | Sequence (relative to DddAtox or wildtype of SEQ ID NO: 25) | SEQ ID NO: |
|---|---|---|
| T1314A/T1380I | GSYALGPYQISAPQLPAYNGQTVGAFYYVNDAGGLESKVFSSGGPTPYP | 28 |
| | NYANAGHVEGQSALFMRDNGISEGLVFHNNPEGTCGFCVNMIETLLPEN | |
| | AKMTVVPPEGAIPVKRGATGETKVFTGNSNSPKSPTKGGC | |
| Q1310R/S1330I/ | GSYALGPYQISAPQLPAYNGRTVGTFYYVNDAGGLESKVFISGGPTPYPN | 29 |
| T1380I | YANAGHVEGQSALFMRDNGISEGLVFHNNPEGTCGFCVNMIETLLPENA | |
| | KMTVVPPEGAIPVKRGATGETKVFTGNSNSPKSPTKGGC | |
| T1380I/T1413I | GSYALGPYQISAPQLPAYNGQTVGTFYYVNDAGGLESKVFSSGGPTPYPN | 30 |
| | YANAGHVEGQSALFMRDNGISEGLVFHNNPEGTCGFCVNMIETLLPENA | |
| | KMTVVPPEGAIPVKRGATGETKVFIGNSNSPKSPTKGGC | |
| T1314A/T1380I/ | GSYALGPYQISAPQLPAYNGQTVGAFYYVNDAGGLESKVFSSGGPTPYP | 31 |
| E1396K | NYANAGHVEGQSALFMRDNGISEGLVFHNNPEGTCGFCVNMIETLLPEN | |
| | AKMTVVPPKGAIPVKRGATGETKVFTGNSNSPKSPTKGGC | |
| Q1310R | GSYALGPYQISAPQLPAYNGRTVGTFYYVNDAGGLESKVFSSGGPTPYPN | 32 |
| | YANAGHVEGQSALFMRDNGISEGLVFHNNPEGTCGFCVNMTETLLPENA | |
| | KMTVVPPEGAIPVKRGATGETKVFTGNSNSPKSPTKGGC | |
| S1330I | GSYALGPYQISAPQLPAYNGQTVGTFYYVNDAGGLESKVFISGGPTPYPN | 33 |
| | YANAGHVEGQSALFMRDNGISEGLVFHNNPEGTCGFCVNMTETLLPENA | |
| | KMTVVPPEGAIPVKRGATGETKVFTGNSNSPKSPTKGGC | |
| Q1310R/S1330I | GSYALGPYQISAPQLPAYNGRTVGTFYYVNDAGGLESKVFISGGPTPYPN | 34 |
| | YANAGHVEGQSALFMRDNGISEGLVFHNNPEGTCGFCVNMTETLLPENA | |
| | KMTVVPPEGAIPVKRGATGETKVFTGNSNSPKSPTKGGC | |
| Q1310R/T1380I | GSYALGPYQISAPQLPAYNGRTVGTFYYVNDAGGLESKVFSSGGPTPYPN | 35 |
| | YANAGHVEGQSALFMRDNGISEGLVFHNNPEGTCGFCVNMIETLLPENA | |
| | KMTVVPPEGAIPVKRGATGETKVFTGNSNSPKSPTKGGC | |
| S1330I/T1380I | GSYALGPYQISAPQLPAYNGQTVGTFYYVNDAGGLESKVFISGGPTPYPN | 36 |
| | YANAGHVEGQSALFMRDNGISEGLVFHNNPEGTCGFCVNMIETLLPENA | |
| | KMTVVPPEGAIPVKRGATGETKVFTGNSNSPKSPTKGGC | |
| T1380I/E1396K | GSYALGPYQISAPQLPAYNGQTVGTFYYVNDAGGLESKVFSSGGPTPYPN | 37 |
| | YANAGHVEGQSALFMRDNGISEGLVFHNNPEGTCGFCVNMIETLLPENA | |
| | KMTVVPPKGAIPVKRGATGETKVFTGNSNSPKSPTKGGC | |
| Q1310R/S1330I/ | GSYALGPYQISAPQLPAYNGRTVGTFYYVNDAGGLESKVFISGGPTPYPN | 38 |
| T1380I/E1396K | YANAGHVEGQSALFMRDNGISEGLVFHNNPEGTCGFCVNMIETLLPENA | |
| | KMTVVPPKGAIPVKRGATGETKVFTGNSNSPKSPTKGGC | |
| Q1310R/T1413I | GSYALGPYQISAPQLPAYNGRTVGTFYYVNDAGGLESKVFSSGGPTPYPN | 39 |
| | YANAGHVEGQSALFMRDNGISEGLVFHNNPEGTCGFCVNMTETLLPENA | |
| | KMTVVPPEGAIPVKRGATGETKVFIGNSNSPKSPTKGGC | |
| S1330I/T1413I | GSYALGPYQISAPQLPAYNGQTVGTFYYVNDAGGLESKVFISGGPTPYPN | 40 |
| | YANAGHVEGQSALFMRDNGISEGLVFHNNPEGTCGFCVNMTETLLPENA | |
| | KMTVVPPEGAIPVKRGATGETKVFIGNSNSPKSPTKGGC | |
| Q1310R/S1330I/ | GSYALGPYQISAPQLPAYNGRTVGTFYYVNDAGGLESKVFISGGPTPYPN | 41 |
| T1413I | YANAGHVEGQSALFMRDNGISEGLVFHNNPEGTCGFCVNMTETLLPENA | |
| | KMTVVPPEGAIPVKRGATGETKVFIGNSNSPKSPTKGGC | |
| Q1310R/T1380I/ | GSYALGPYQISAPQLPAYNGRTVGTFYYVNDAGGLESKVFSSGGPTPYPN | 42 |
| T1413I | YANAGHVEGQSALFMRDNGISEGLVFHNNPEGTCGFCVNMIETLLPENA | |
| | KMTVVPPEGAIPVKRGATGETKVFIGNSNSPKSPTKGGC | |
| S1330I/T1380I/ | GSYALGPYQISAPQLPAYNGQTVGTFYYVNDAGGLESKVFISGGPTPYPN | 43 |
| T1413I | YANAGHVEGQSALFMRDNGISEGLVFHNNPEGTCGFCVNMIETLLPENA | |
| (III) | KMTVVPPEGAIPVKRGATGETKVFIGNSNSPKSPTKGGC | |
| T1380I/E1396K/ | GSYALGPYQISAPQLPAYNGQTVGTFYYVNDAGGLESKVFSSGGPTPYPN | 44 |
| T1413I | YANAGHVEGQSALFMRDNGISEGLVFHNNPEGTCGFCVNMIETLLPENA | |
| | KMTVVPPKGAIPVKRGATGETKVFIGNSNSPKSPTKGGC | |
| Q1310R/S1330I/ | GSYALGPYQISAPQLPAYNGRTVGTFYYVNDAGGLESKVFISGGPTPYPN | 45 |
| T1380I/T1413I | YANAGHVEGQSALFMRDNGISEGLVFHNNPEGTCGFCVNMIETLLPENA | |
| (RIII) | KMTVVPPEGAIPVKRGATGETKVFIGNSNSPKSPTKGGC | |
| Q1310R/S1330I/ | GSYALGPYQISAPQLPAYNGRTVGTFYYVNDAGGLESKVFISGGPTPYPN | 46 |
| T1380I/E1396K/ | YANAGHVEGQSALFMRDNGISEGLVFHNNPEGTCGFCVNMIETLLPENA | |
| T1413I | KMTVVPPKGAIPVKRGATGETKVFIGNSNSPKSPTKGGC | |
| T1314A/G1344R/ | GSYALGPYQISAPQLPAYNGQTVGAFYYVNDAGGLESKVFSSGGPTPYP | 47 |
| V1364M/ | NYANARHVEGQSALFMRDNGISEGLMFHNNPKGTCGFCVNMIETLLPEN | |
| E1370K/T1380I/ | AKMTVVPPEGAIPVKRGATGETKVFIGNSNSPKSPTKGGC | |
| T1413I | | |
| (CC1) | | |
| N1342S/G1344R/ | GSYALGPYQISAPQLPAYNGQTVGTFYYVNDAGGLESKVFSSGGPTPYPN | 48 |
| V1364M/ | YASARHVEGQSALFMRDNGISEGLMFHNNPKGTCGFCVNMIETLLPENA | |
| E1370K/ | | |
| T1380I/T1413I | | |
| (CC2) | | |
| A1341T/N1342S/ | GSYALGPYQISAPQLPAYNGQTVGTFYYVNDAGGLESKVFSSGGPTPYPN | 49 |
| E1370K/T1380I/ | YTSAGHVEGQSALFMRDNGISEGLVFHNNPKGTCGFCVNMIETLLPENA | |
| E1408K | KMTVVPPEGAIPVKRGATGKTKVFTGNSNSPKSPTKGGC | |
| (CC3) | | |
| A1341T/N1342S/ | GSYALGPYQISAPQLPAYNGQTVGTFYYVNDAGGLESKVFSSGGPTPYPN | 50 |
| E1370K/T1380I/ | YTSAGHVEGQSALFMRDNGISEGLVFHNNPKGTCGFCVNMIETLLPENA | |
| E1408K/T1413I | | |
| (CC3a) | | |
| A1341T/N1342S/ | GSYALGPYQISAPQLPAYNGQTVGTFYYVNDAGGLESKVFSSGGPTPYPN | 51 |
| E1370K/T1380I/ | YTSAGHVEGQSALFMRDNGISEGLVFHNNPKGTCGFCVNMIETLLPENA | |
| E1408G/T1413I | | |
| (CC3b) | | |
| T1314/G1344S/ | GSYALGPYQISAPQLPAYNGQTVGAFYYVNDAGGLESKVFSSGGPTPYP | 52 |
| E1370K/T1380I/ | NYANASHVEGQSALFMRDNGISEGLVFHNNPKGTCGFCVNMIETLLPEN | |
| A1398T/T1413I | AKMTVVPPEGTIPVKRGATGETKVFIGNSNSPKSPTKGGC | |
| (GC1) | | |
| T1314/G1344S/ | GSYALGPYQISAPQLPAYNGQTVGAFYYVNDAGGLESKVFSSGGPTPYP | 53 |
| E1370K/T1380I/ | NYANASHVEGQSALFMRDNGISEGLVFHNNPKGTCGFCVNMIETLLPEN | |
| E1396K/A1398T/ | AKMTVVPPKGTIPVKRGATGETKVFIGNSNSPKSPTKGGC | |
| T1413I | | |
| (GC2) | | |
| S1330I/A1341V/ | GSYALGPYQISAPQLPAYNGQTVGTFYYVNDAGGLESKVFISGGPTPYPN | 54 |
| N1342S/E1370K/ | YVSAGHVEGQSALFMRDNGISEGLVFHNNPKGTCGFCVNMIETLLPENA | |
| T1380I/T1413I | KMTVVPPEGAIPVKRGATGETKVFIGNSNSPKSPTKGGC | |
| (GC3) | | |

### II. Programmable DNA Binding Protein

In various embodiments, the Evolved DddA-containing base editors or the polypeptides that comprise the Evolved DddA-containing base editors (e.g., the pDNAbps and DddA) may include a programmable DNA binding protein, such as a mitoTALE, zinc finger protein, or napDNAbp (e.g., Cas9).

### MitoTALEs and MitoZFs

In various embodiments, the Evolved DddA-containing base editors or the polypeptides that comprise the Evolved DddA-containing base editors (e.g., the pDNAbps and DddA) may include a mitoTALE as the pDNAbp component.

MitoTALEs and mitoZFP are known in the art. Each of the proteins may comprise a mitochondrial targeting sequence (MTS) in order to facilitate the translocation of the protein into the mitochondria.

In one aspect, the methods and compositions described herein involve a TALE protein programmed (e.g., engineered through manipulation of the localization signal in the C-terminus) to localize to the mitochondria (mitoTALE). In some embodiments, the localization signal comprises a sequence to target SOD2. In some embodiments, the LS comprises SEQ ID NO: 13. In some embodiments, the LS comprises a sequence to target Cox8a. In some embodiments, the LS comprises SEQ ID NO.: 14. In some embodiments, the LS comprises a sequence with 75% or greater percent identity (e.g., 80% or greater, 85% or greater, 90% or greater, 95% or greater, 96% or greater, 97% or greater, 98% or greater, 99% or greater, 99.5% or greater, 99.9% or greater percent identity) to SEQ ID NOs.: 13 or 14.

The mitoTALE is also used to guide the fusion protein to the appropriate target nucleotide in the mtDNA. By using the RVD in the mitoTALE specific sequences can be targeted, which will place the attached DddA proximal to the target nucleotide. As used herein, “proximal” or “proximally” with respect to a target nucleotide shall mean a range of nucleic acids which are arranged consecutively upstream or downstream of the target nucleotide, on either the strand containing the target nucleotide or the strand complementary to the strand containing the target nucleotide, which when targeted and bound by a mitoTALE allow for the dimerization or re-assembly of portions of a DddA to regain, at least partially, the native activity of a full length DddA. Accordingly, the sequence should be selected from a range of nucleotides at or near the target nucleotide, or the nucleotide complementary thereto. In some embodiments, the target nucleic acid sequence is located upstream of the target nucleotide. In some embodiments, the target nucleic acid sequence is between 1 and 40 nucleotides upstream of the target nucleotide. In some embodiments, the target nucleic acid sequence is between 5 and 20 nucleotides upstream of the target nucleotide.

In some embodiments, a second mitoTALE is used. A second mitoTALE can be used to deliver additional components (e.g., additional DddA, a second portion of a DddA, additional enzymes). In some embodiments, the second mitoTALE is configured to bind a second target nucleic acid sequence. In some embodiments, the second mitoTALE is configured to bind a second target nucleic acid sequence on the nucleic acid strand complementary to the strand containing the target nucleotide. In some embodiments, the second mitoTALE is configured to bind a second target nucleic acid sequence upstream of the nucleotide complementary to the target nucleotide, which complementary nucleotide is on the nucleic acid strand complementary to the strand containing the target nucleotide. In some embodiments, the second target nucleic acid sequence is between 1 and 40 nucleotides upstream of the nucleotide complementary to the target nucleotide, which is on the strand complementary to the strand containing the target nucleotide. In some embodiments, the second target nucleic acid sequence is between 5 and 20 nucleotides upstream of the nucleotide complementary to the target nucleotide, which is on the strand complementary to the strand containing the target nucleotide.

In some embodiments, a mitoTALE comprises an amino acid sequence selected from any one of the following amino acid sequences, or an amino acid sequence having at least 70%, 75%, 80%, 85%, 90%, 95%, or 99% sequence identity with any one of the following mitoTALE sequences:

| SEQ ID NO: | Sequence | Description |
|---|---|---|
| 1 | MALSRAVCGTSRQLAPVLGYLGSRQKHSLPDYPYDVPDYAGYPYDVPDYAGYPY | Mito 24 |
| | DVPDYAMDIADLRTLGYSQQQQEKIKPKVRSTVAQHHEALVGHGFTHAHIVALSQ | |
| | HPAALGTVAVKYQDMIAALPEATHEAIVGVGKQWSGARALEALLTVAGELRGPPL | |
| | QLDTGQLLKIAKRGGVTAVEAVHAWRNALTGAPLNLTPQQVVAIASNNGGKQAL | |
| | ETVQRLLPVLCQAHGLTPEQVVAIASNIGGKQALETVQALLPVLCQAHGLTPEQVV | |
| | AIASHDGGKQALETVQRLLPVLCQAHGLTPEQVVAIASHDGGKQALETVQRLLPV | |
| | LCQAHGLTPEQVVAIASHDGGKQALETVQRLLPVLCQAHGLTPEQVVAIASHDGG | |
| | KQALETVQRLLPVLCQAHGLTPEQVVAIASHDGGKQALETVQRLLPVLCQAHGLT | |
| | PEQVVAIASNIGGKQALETVQALLPVLCQAHGLTPQQVVAIASNGGGKQALETVQ | |
| | RLLPVLCQAHGLTPQQVVAIASNGGGRPALESIVAQLSRPDPALAALTNDHLVALA | |
| | CLGGRPALDAVKKGLGGSGSYALGPYQISAPQLPAYNGQTVGTFYYVNDAGGLES | |
| | KVFSSGGPTPYPNYANAGHVEGQSALFMRDNGISEGLVFHNNPEGTCGFCVNMTE | |
| | TLLPENAKMTVVPPEG | |
| 2 | MALSRAVCGTSRQLAPVLGYLGSRQKHSLPDYPYDVPDYAGYPYDVPDYAGYPY | Mito 24a |
| | DVPDYATNLSDIIEKETGKQLVIQESILMLPEEVEEVIGNKPESDILVHTAYDESTDE | |
| | NVMLLTSDAPEYKPWALVIQDSNGENKIKMLSGGSGGSGGSTNLSDIIEKETGKQL | |
| | VIQESILMLPEEVEEVIGNKPESDILVHTAYDESTDENVMLLTSDAPEYKPWALVIQ | |
| | DSNGENKIKMLSGGSMDIADLRTLGYSQQQQEKIKPKVRSTVAQHHEALVGHGFT | |
| | HAHIVALSQHPAALGTVAVKYQDMIAALPEATHEAIVGVGKQWSGARALEALLTV | |
| | AGELRGPPLQLDTGQLLKIAKRGGVTAVEAVHAWRNALTGAPLNLTPQQVVAIAS | |
| | NNGGKQALETVQRLLPVLCQAHGLTPEQVVAIASNIGGKQALETVQALLPVLCQA | |
| | HGLTPEQVVAIASHDGGKQALETVQRLLPVLCQAHGLTPEQVVAIASHDGGKQAL | |
| | ETVQRLLPVLCQAHGLTPEQVVAIASHDGGKQALETVQRLLPVLCQAHGLTPEQV | |
| | VAIASHDGGKQALETVQRLLPVLCQAHGLTPEQVVAIASHDGGKQALETVQRLLP | |
| | VLCQAHGLTPEQVVAIASNIGGKQALETVQALLPVLCQAHGLTPQQVVAIASNGG | |
| | GKQALETVQRLLPVLCQAHGLTPQQVVAIASNGGGRPALESIVAQLSRPDPALAAL | |
| | TNDHLVALACLGGRPALDAVKKGLGGSGSYALGPYQISAPQLPAYNGQTVGTFYY | |
| | VNDAGGLESKVFSSGGPTPYPNYANAGHVEGQSALFMRDNGISEGLVFHNNPEGT | |
| | CGFCVNMTETLLPENAKMTVVPPEG | |
| 3 | MALSRAVCGTSRQLAPVLGYLGSRQKHSLPDYPYDVPDYAGYPYDVPDYAGYPY | Mito 24b |
| | DVPDYAMDIADLRTLGYSQQQQEKIKPKVRSTVAQHHEALVGHGFTHAHIVALSQ | |
| | HPAALGTVAVKYQDMIAALPEATHEAIVGVGKQWSGARALEALLTVAGELRGPPL | |
| | QLDTGQLLKIAKRGGVTAVEAVHAWRNALTGAPLNLTPQQVVAIASNNGGKQAL | |
| | ETVQRLLPVLCQAHGLTPEQVVAIASNIGGKQALETVQALLPVLCQAHGLTPEQVV | |
| | AIASHDGGKQALETVQRLLPVLCQAHGLTPEQVVAIASHDGGKQALETVQRLLPV | |
| | LCQAHGLTPEQVVAIASHDGGKQALETVQRLLPVLCQAHGLTPEQVVAIASHDGG | |
| | KQALETVQRLLPVLCQAHGLTPEQVVAIASHDGGKQALETVQRLLPVLCQAHGLT | |
| | PEQVVAIASNIGGKQALETVQALLPVLCQAHGLTPQQVVAIASNGGGKQALETVQ | |
| | RLLPVLCQAHGLTPQQVVAIASNGGGRPALESIVAQLSRPDPALAALTNDHLVALA | |
| | CLGGRPALDAVKKGLGGSGSYALGPYQISAPQLPAYNGQTVGTFYYVNDAGGLES | |
| | KVFSSGGPTPYPNYANAGHVEGQSALFMRDNGISEGLVFHNNPEGTCGFCVNMTE | |
| | TLLPENAKMTVVPPEGSGGSTNLSDIIEKETGKQLVIQESILMLPEEVEEVIGNKPES | |
| | DILVHTAYDESTDENVMLLTSDAPEYKPWALVIQDSNGENKIKMLSGGSGGSGGST | |
| | NLSDIIEKETGKQLVIQESILMLPEEVEEVIGNKPESDILVHTAYDESTDENVMLLTS | |
| | DAPEYKPWALVIQDSNGENKIKML | |
| 4 | MALSRAVCGTSRQLAPVLGYLGSRQKHSLPDYPYDVPDYAGYPYDVPDYAGYPY | Mito 24c |
| | DVPDYATNLSDIIEKETGKQLVIQESILMLPEEVEEVIGNKPESDILVHTAYDESTDE | |
| | NVMLLTSDAPEYKPWALVIQDSNGENKIKMLSGGSMDIADLRTLGYSQQQQEKIK | |
| | PKVRSTVAQHHEALVGHGFTHAHIVALSQHPAALGTVAVKYQDMIAALPEATHEA | |
| | IVGVGKQWSGARALEALLTVAGELRGPPLQLDTGQLLKIAKRGGVTAVEAVHAW | |
| | RNALTGAPLNLTPQQVVAIASNNGGKQALETVQRLLPVLCQAHGLTPEQVVAIAS | |
| | NIGGKQALETVQALLPVLCQAHGLTPEQVVAIASHDGGKQALETVQRLLPVLCQA | |
| | HGLTPEQVVAIASHDGGKQALETVQRLLPVLCQAHGLTPEQVVAIASHDGGKQAL | |
| | ETVQRLLPVLCQAHGLTPEQVVAIASHDGGKQALETVQRLLPVLCQAHGLTPEQV | |
| | VAIASHDGGKQALETVQRLLPVLCQAHGLTPEQVVAIASNIGGKQALETVQALLPV | |
| | LCQAHGLTPQQVVAIASNGGGKQALETVQRLLPVLCQAHGLTPQQVVAIASNGGG | |
| | RPALESIVAQLSRPDPALAALTNDHLVALACLGGRPALDAVKKGLGGSGSYALGP | |
| | YQISAPQLPAYNGQTVGTFYYVNDAGGLESKVFSSGGPTPYPNYANAGHVEGQSA | |
| | LFMRDNGISEGLVFHNNPEGTCGFCVNMTETLLPENAKMTVVPPEG | |
| 5 | MALSRAVCGTSRQLAPVLGYLGSRQKHSLPDYPYDVPDYAGYPYDVPDYAGYPY | Mito 24d |
| | DVPDYAMDIADLRTLGYSQQQQEKIKPKVRSTVAQHHEALVGHGFTHAHIVALSQ | |
| | HPAALGTVAVKYQDMIAALPEATHEAIVGVGKQWSGARALEALLTVAGELRGPPL | |
| | QLDTGQLLKIAKRGGVTAVEAVHAWRNALTGAPLNLTPQQVVAIASNNGGKQAL | |
| | ETVQRLLPVLCQAHGLTPEQVVAIASNIGGKQALETVQALLPVLCQAHGLTPEQVV | |
| | AIASHDGGKQALETVQRLLPVLCQAHGLTPEQVVAIASHDGGKQALETVQRLLPV | |
| | LCQAHGLTPEQVVAIASHDGGKQALETVQRLLPVLCQAHGLTPEQVVAIASHDGG | |
| | KQALETVQRLLPVLCQAHGLTPEQVVAIASHDGGKQALETVQRLLPVLCQAHGLT | |
| | PEQVVAIASNIGGKQALETVQALLPVLCQAHGLTPQQVVAIASNGGGKQALETVQ | |
| | RLLPVLCQAHGLTPQQVVAIASNGGGRPALESIVAQLSRPDPALAALTNDHLVALA | |
| | CLGGRPALDAVKKGLGGSGSYALGPYQISAPQLPAYNGQTVGTFYYVNDAGGLES | |
| | KVFSSGGPTPYPNYANAGHVEGQSALFMRDNGISEGLVFHNNPEGTCGFCVNMTE | |
| | TLLPENAKMTVVPPEGSGGSTNLSDIIEKETGKQLVIQESILMLPEEVEEVIGNKPES | |
| | DILVHTAYDESTDENVMLLTSDAPEYKPWALVIQDSNGENKIKML | |
| 6 | MASVLTPLLLRGLTGSARRLPVPRAKIHSLDYKDHDGDYKDHDIDYKDDDDKMDI | Mito 28 |
| | ADLRTLGYSQQQQEKIKPKVRSTVAQHHEALVGHGFTHAHIVALSQHPAALGTVA | |
| | VKYQDMIAALPEATHEAIVGVGKRGAGARALEALLTVAGELRGPPLQLDTGQLLK | |
| | IAKRGGVTAVEAVHAWRNALTGAPLNLTPQQVVAIASNNGGKQALETVQRLLPVL | |
| | CQAHGLTPEQVVAIASNIGGKQALETVQALLPVLCQAHGLTPQQVVAIASNGGGK | |
| | QALETVQRLLPVLCQAHGLTPQQVVAIASNNGGKQALETVQRLLPVLCQAHGLTP | |
| | QQVVAIASNNGGKQALETVQRLLPVLCQAHGLTPEQVVAIASHDGGKQALETVQR | |
| | LLPVLCQAHGLTPQQVVAIASNGGGKQALETVQRLLPVLCQAHGLTPEQVVAIAS | |
| | NIGGKQALETVQALLPVLCQAHGLTPQQVVAIASNGGGKQALETVQRLLPVLCQA | |
| | HGLTPQQVVAIASNGGGKQALETVQRLLPVLCQAHGLTPQQVVAIASNGGGRPAL | |
| | ESIVAQLSRPDPALAALTNDHLVALACLGGRPALDAVKKGLGGSAIPVKRGATGET | |
| | KVFTGNSNSPKSPTKGGC | |
| 7 | MASVLTPLLLRGLTGSARRLPVPRAKIHSLDYKDHDGDYKDHDIDYKDDDDKTNL | Mito 28a |
| | SDIIEKETGKQLVIQESILMLPEEVEEVIGNKPESDILVHTAYDESTDENVMLLTSDA | |
| | PEYKPWALVIQDSNGENKIKMLSGGSGGSGGSTNLSDIIEKETGKQLVIQESILMLPE | |
| | EVEEVIGNKPESDILVHTAYDESTDENVMLLTSDAPEYKPWALVIQDSNGENKIKM | |
| | LSGGSMDIADLRTLGYSQQQQEKIKPKVRSTVAQHHEALVGHGFTHAHIVALSQHP | |
| | AALGTVAVKYQDMIAALPEATHEAIVGVGKRGAGARALEALLTVAGELRGPPLQL | |
| | DTGQLLKIAKRGGVTAVEAVHAWRNALTGAPLNLTPQQVVAIASNNGGKQALET | |
| | VQRLLPVLCQAHGLTPEQVVAIASNIGGKQALETVQALLPVLCQAHGLTPQQVVAI | |
| | ASNGGGKQALETVQRLLPVLCQAHGLTPQQVVAIASNNGGKQALETVQRLLPVLC | |
| | QAHGLTPQQVVAIASNNGGKQALETVQRLLPVLCQAHGLTPEQVVAIASHDGGKQ | |
| | ALETVQRLLPVLCQAHGLTPQQVVAIASNGGGKQALETVQRLLPVLCQAHGLTPE | |
| | QVVAIASNIGGKQALETVQALLPVLCQAHGLTPQQVVAIASNGGGKQALETVQRL | |
| | LPVLCQAHGLTPQQVVAIASNGGGKQALETVQRLLPVLCQAHGLTPQQVVAIASN | |
| | GGGRPALESIVAQLSRPDPALAALTNDHLVALACLGGRPALDAVKKGLGGSAIPVK | |
| | RGATGETKVFTGNSNSPKSPTKGGC | |
| 8 | MASVLTPLLLRGLTGSARRLPVPRAKIHSLDYKDHDGDYKDHDIDYKDDDDKMDI | Mito 28b |
| | ADLRTLGYSQQQQEKIKPKVRSTVAQHHEALVGHGFTHAHIVALSQHPAALGTVA | |
| | VKYQDMIAALPEATHEAIVGVGKRGAGARALEALLTVAGELRGPPLQLDTGQLLK | |
| | IAKRGGVTAVEAVHAWRNALTGAPLNLTPQQVVAIASNNGGKQALETVQRLLPVL | |
| | CQAHGLTPEQVVAIASNIGGKQALETVQALLPVLCQAHGLTPQQVVAIASNGGGK | |
| | QALETVQRLLPVLCQAHGLTPQQVVAIASNNGGKQALETVQRLLPVLCQAHGLTP | |
| | QQVVAIASNNGGKQALETVQRLLPVLCQAHGLTPEQVVAIASHDGGKQALETVQR | |
| | LLPVLCQAHGLTPQQVVAIASNGGGKQALETVQRLLPVLCQAHGLTPEQVVAIAS | |
| | NIGGKQALETVQALLPVLCQAHGLTPQQVVAIASNGGGKQALETVQRLLPVLCQA | |
| | HGLTPQQVVAIASNGGGKQALETVQRLLPVLCQAHGLTPQQVVAIASNGGGRPAL | |
| | ESIVAQLSRPDPALAALTNDHLVALACLGGRPALDAVKKGLGGSAIPVKRGATGET | |
| | KVFTGNSNSPKSPTKGGCSGGSTNLSDIIEKETGKQLVIQESILMLPEEVEEVIGNKP | |
| | ESDILVHTAYDESTDENVMLLTSDAPEYKPWALVIQDSNGENKIKMLSGGSGGSGG | |
| | STNLSDIIEKETGKQLVIQESILMLPEEVEEVIGNKPESDILVHTAYDESTDENVMLL | |
| | TSDAPEYKPWALVIQDSNGENKIKML | |
| 9 | MASVLTPLLLRGLTGSARRLPVPRAKIHSLDYKDHDGDYKDHDIDYKDDDDKTNL | Mito 28c |
| | SDIIEKETGKQLVIQESILMLPEEVEEVIGNKPESDILVHTAYDESTDENVMLLTSDA | |
| | PEYKPWALVIQDSNGENKIKMLSGGSMDIADLRTLGYSQQQQEKIKPKVRSTVAQ | |
| | HHEALVGHGFTHAHIVALSQHPAALGTVAVKYQDMIAALPEATHEAIVGVGKRGA | |
| | GARALEALLTVAGELRGPPLQLDTGQLLKIAKRGGVTAVEAVHAWRNALTGAPL | |
| | NLTPQQVVAIASNNGGKQALETVQRLLPVLCQAHGLTPEQVVAIASNIGGKQALET | |
| | VQALLPVLCQAHGLTPQQVVAIASNGGGKQALETVQRLLPVLCQAHGLTPQQVVA | |
| | IASNNGGKQALETVQRLLPVLCQAHGLTPQQVVAIASNNGGKQALETVQRLLPVL | |
| | CQAHGLTPEQVVAIASHDGGKQALETVQRLLPVLCQAHGLTPQQVVAIASNGGGK | |
| | QALETVQRLLPVLCQAHGLTPEQVVAIASNIGGKQALETVQALLPVLCQAHGLTPQ | |
| | QVVAIASNGGGKQALETVQRLLPVLCQAHGLTPQQVVAIASNGGGKQALETVQRL | |
| | LPVLCQAHGLTPQQVVAIASNGGGRPALESIVAQLSRPDPALAALTNDHLVALACL | |
| | GGRPALDAVKKGLGGSAIPVKRGATGETKVFTGNSNSPKSPTKGGC | |
| 10 | MASVLTPLLLRGLTGSARRLPVPRAKIHSLDYKDHDGDYKDHDIDYKDDDDKMDI | Mito 28d |
| | ADLRTLGYSQQQQEKIKPKVRSTVAQHHEALVGHGFTHAHIVALSQHPAALGTVA | |
| | VKYQDMIAALPEATHEAIVGVGKRGAGARALEALLTVAGELRGPPLQLDTGQLLK | |
| | IAKRGGVTAVEAVHAWRNALTGAPLNLTPQQVVAIASNNGGKQALETVQRLLPVL | |
| | CQAHGLTPEQVVAIASNIGGKQALETVQALLPVLCQAHGLTPQQVVAIASNGGGK | |
| | QALETVQRLLPVLCQAHGLTPQQVVAIASNNGGKQALETVQRLLPVLCQAHGLTP | |
| | QQVVAIASNNGGKQALETVQRLLPVLCQAHGLTPEQVVAIASHDGGKQALETVQR | |
| | LLPVLCQAHGLTPQQVVAIASNGGGKQALETVQRLLPVLCQAHGLTPEQVVAIAS | |
| | NIGGKQALETVQALLPVLCQAHGLTPQQVVAIASNGGGKQALETVQRLLPVLCQA | |
| | HGLTPQQVVAIASNGGGKQALETVQRLLPVLCQAHGLTPQQVVAIASNGGGRPAL | |
| | ESIVAQLSRPDPALAALTNDHLVALACLGGRPALDAVKKGLGGSAIPVKRGATGET | |
| | KVFTGNSNSPKSPTKGGCSGGSTNLSDIIEKETGKQLVIQESILMLPEEVEEVIGNKP | |
| | ESDILVHTAYDESTDENVMLLTSDAPEYKPWALVIQDSNGENKIKML | |
| 11 | DIADLRTLGYSQQQQEKIKPKVRSTVAQHHEALVGHGFTHAHIVALSQHPAALGT | Right |
| | VAVKYQDMIAALPEATHEAIVGVGKQWSGARALEALLTVAGELRGPPLQLDTGQL | Modified m.13513- |
| | LKIAKRGGVTAVEAVHAWRNALTGAPLNLTPQQVVAIASHDGGKQALETVQRLLP | TALE |
| | VLCQAHGLTPEQVVAIASNGGGKQALETVQRLLPVLCQAHGLTPQQVVAIASNGG | |
| | GKQALETVQRLLPVLCQAHGLTPEQVVAIASNGGGKQALETVQALLPVLCQAHGL | |
| | TPQQVVAIASNNGGKQALETVQRLLPVLCQAHGLTPQQVVAIASNNGGKQALETV | |
| | QRLLPVLCQAHGLTPEQVVAIASNIGGKQALETVQRLLPVLCQAHGLTPQQVVAIA | |
| | SNNGGKQALETVQRLLPVLCQAHGLTPQQVVAIASNGGGKQALETVQRLLPVLCQ | |
| | AHGLTPEQVVAIASNIGGKQALETVQRLLPVLCQAHGLTPEQVVAIASNNGGKQAL | |
| | ETVQRLLPVLCQAHGLTPQQVVAIASNIGGKQALETVQRLLPVLCQAHGLTPQQV | |
| | VAIASNIGGRPALESIVAQLSRPDPALAALTNDHLVALACLGGRPALDAVKKGLG | |
| 12 | DIADLRTLGYSQQQQEKIKPKVRSTVAQHHEALVGHGFTHAHIVALSQHPAALGT | m.8490- |
| | VAVKYQDMIAALPEATHEAIVGVGKQWSGARALEALLTVAGELRGPPLQLDTGQL | Right |
| | LKIAKRGGVTAVEAVHAWRNALTGAPLNLTPQQVVAIASNIGGKQALETVQRLLP | TALE |
| | VLCQAHGLTPEQVVAIASNGGGKQALETVQRLLPVLCQAHGLTPQQVVAIASNNG | |
| | GKQALETVQRLLPVLCQAHGLTPEQVVAIASNNGGKQALETVQALLPVLCQAHGL | |
| | TPQQVVAIASNNGGKQALETVQRLLPVLCQAHGLTPQQVVAIASHDGGKQALETV | |
| | QRLLPVLCQAHGLTPEQVVAIASNGGGKQALETVQRLLPVLCQAHGLTPQQVVAI | |
| | ASNGGGKQALETVQRLLPVLCQAHGLTPQQVVAIASNGGGKQALETVQRLLPVLC | |
| | QAHGLTPEQVVAIASNNGGKQALETVQRLLPVLCQAHGLTPEQVVAIASNNGGKQ | |
| | ALETVQRLLPVLCQAHGLTPQQVVAIASNGGGRPALESIVAQLSRPDPALAALTND | |
| | HLVALACLGGRPALDAVKKGLG | |
| 13 | MLSRAVCGTSRQLAPVLGYLGSRQKHSLPD | SOD2 MLS |
| 14 | MSVLTPLLLRGLTGSARRLPVPRAKIHSL | COX8a |
| | | MLS |
| 15 | ACCACGATCGTTATGCTGATCATACCCTAATGATCCCAGCAAGATAATGTCCTG | SOD2 |
| | TCTTCTAAGATGTGCATCAAGCCTGGTACATACTGAAAACCCTATAAGGTCCTG | 3′UTR |
| | GATAATTTTTGTTTGATTATTCATTGAAGAAACATTTATTTTCCAATTGTGTGAA | |
| | GTTTTTGACTGTTAATAAAAGAATCTGTCAACCATCAAAAAAAAAAAAAAA | |
| 15 | ACCACGATCGTTATGCTGATCATACCCTAATGATCCCAGCAAGATAATGTCCTG | ATP5b |
| | TCTTCTAAGATGTGCATCAAGCCTGGTACATACTGAAAACCCTATAAGGTCCTG | 3′UTR |
| | GATAATTTTTGTTTGATTATTCATTGAAGAAACATTTATTTTCCAATTGTGTGAA | |
| | GTTTTTGACTGTTAATAAAAGAATCTGTCAACCATCAAAAAAAAAAAAAAA | |

In addition, the mitoTALE and/or mitoZFP may comprising one of the following mitochondrial targeting sequences which help promote mitochondrial localization, or an amino acid or nucleotide sequence having at least 70%, 75%, 80%, 85%, 90%, 95%, or 99% sequence identity with any one of the following sequences:

| SEQ ID NO: | Sequence | Description |
|---|---|---|
| 13 | MLSRAVCGTSRQLAPVLGYLGSRQKHSLPD | SOD2 (mitochondrial |
| | | superoxide dismutase) MTS |
| 14 | MSVLTPLLLRGLTGSARRLPVPRAKIHSL | COX8a (mitochondrial |
| | | cytochrome C oxidase |
| | | subunit 8A) MTS |
| 15 | ACCACGATCGTTATGCTGATCATACCCTAATGATCCCAGCAAGA | SOD2 3′UTR |
| | TAATGTCCTGTCTTCTAAGATGTGCATCAAGCCTGGTACATACT | |
| | GAAAACCCTATAAGGTCCTGGATAATTTTTGTTTGATTATTCATT | |
| | GAAGAAACATTTATTTTCCAATTGTGTGAAGTTTTTGACTGTTA | |
| | ATAAAAGAATCTGTCAACCATCAAAAAAAAAAAAAAA | |
| 15 | ACCACGATCGTTATGCTGATCATACCCTAATGATCCCAGCAAGA | ATP5b 3′UTR |
| | TAATGTCCTGTCTTCTAAGATGTGCATCAAGCCTGGTACATACT | |
| | GAAAACCCTATAAGGTCCTGGATAATTTTTGTTTGATTATTCATT | |
| | GAAGAAACATTTATTTTCCAATTGTGTGAAGTTTTTGACTGTTA | |
| | ATAAAAGAATCTGTCAACCATCAAAAAAAAAAAAAAA | |

In various embodiments, the Evolved DddA-containing base editors may comprises a mitoZF. A mitoZF may be a ZF protein comprising one or more mitochondrial localization sequences (MLS). A zinc finger is a small, functional, independently folded domain that coordinates one or more zinc ions to stabilize its structure through cysteine and/or histidine residues. Zinc fingers are structurally diverse and exhibit a wide range of functions, from DNA- or RNA-binding to protein-protein interactions and membrane association. There are more than 40 types of zinc fingers annotated in UniProtKB. The most frequent are the C2H2-type, the CCHC-type, the PHD-type and the RING-type. Examples include Accession Nos. Q7Z142, P55197, Q9P2R3, Q9P2G1, Q9P2S6, Q8IUH5, P19811, Q92793, P36406, 095081, and Q9ULV3, some of which have the following sequences:

Zinc finger protein: Q7Z142-1:
(SEQ ID NO: 406)
MPDFTIIQPDRKFDAAAVAGIFVRSSTSSSFPSASSYIAAKKRKNVDNTS
TRKPYSYKDRKRKNTEEIRNIKKKLFMDLGIVRTNCGIDNEKQDREKAMK
RKVTETIVTTYCELCEQNFSSSKMLLLHRGKVHNTPYIECHLCMKLFSQT
IQFNRHMKTHYGPNAKIYVQCELCDRQFKDKQSLRTHWDVSHGSGDNQAV
LA,

or an amino acid having at least 70%, 75%, 80%, 85%, 90%, 95%, or 99% sequence identity therewith, or fragment thereof.

Zinc finger protein: P55197-4 (isoform-4):
(SEQ ID NO: 407)
MVSSDRPVSLEDEVSHSMKEMIGGCCVCSDERGWAENPLVYCDGHGCSVA
VHQACYGIVQVPTGPWFCRKCESQERAARVRCELCPHKDGALKRTDNGGW
AHVVCALYIPEVQFANVSTMEPIVLQSVPHDRYNKTCYICDEQGRESKAA
TGACMTCNKHGCRQAFHVTCAQFAGLLCEEEGNGADNVQYCGYCKYHFSK
LKKSKRGSNRSYDQSLSDSSSHSQDKHHEKEKKKYKEKDKHKQKHKKQPE
PSPALVPSLTVTTEKTYTSTSNNSISGSLKRLEDTTARFTNANFQEVSAH
TSSGKDVSETRGSEGKGKKSSAHSSGQRGRKPGGGRNPGTTVSAASPFPQ
GSFSGTPGSVKSSSGSSVQSPQDFLSFTDSDLRNDSYSHSQQSSATKDVH
KGESGSQEGGVNSFSTLIGLPSTSAVTSQPKSFENSPGDLGNSSLPTAGY
KRAQTSGIEEETVKEKKRKGNKQSKHGPGRPKGNKNQENVSHLSVSSASP
TSSVASAAGSITSSSLQKSPTLLRNGSLQSLSVGSSPVGSEISMQYRHDG
ACPTTTFSELLNAIHNGIYNSNDVAVSFPNVVSGSGSSTPVSSSHLPQQS
SGHLQQVGALSPSAVSSAAPAVATTQANTLSGSSLSQAPSHMYGNRSNSS
MAALIAQSENNQTDQDLGDNSRNLVGRGSSPRGSLSPRSPVSSLQIRYDQ
PGNSSLENLPPVAASIEQLLERQWSEGQQFLLEQGTPSDILGMLKSLHQL
QVENRRLEEQIKNLTAKKERLQLLNAQLSVPFPTITANPSPSHQIHTFSA
QTAPTTDSLNSSKSPHIGNSFLPDNSLPVLNQDLTSSGQSTSSSSALSTP
PPAGQSPAQQGSGVSGVQQVNGVTVGALASGMQPVTSTIPAVSAVGGIIG
ALPGNQLAINGIVGALNGVMQTPVTMSQNPTPLTHTTVPPNATHPMPATL
TNSASGLGLLSDQQRQILIHQQQFQQLLNSQQLTPEQHQAFLYQLMQHHH
QQHHQPELQQLQIPGPTQIPINNLLAGTQAPPLHTATTNPFLTIHGDNAS
QKVARLSDKTGPVAQEKS,

or an amino acid having at least 70%, 75%, 80%, 85%, 90%, 95%, or 99% sequence identity therewith, or fragment thereof.

Zinc finger protein: Q9P2R3-1 (isoform 1):
(SEQ ID NO: 408)
MAEEEVAKLEKHLMLLRQEYVKLQKKLAETEKRCALLAAQANKESSSESF
ISRLLAIVADLYEQEQYSDLKIKVGDRHISAHKFVLAARSDSWSLANLSS
TKELDLSDANPEVTMTMLRWIYTDELEFREDDVFLTELMKLANRFQLQLL
RERCEKGVMSLVNVRNCIRFYQTAEELNASTLMNYCAEIIASHWDDLRKE
DFSSMSAQLLYKMIKSKTEYPLHKAIKVEREDVVFLYLIEMDSQLPGKLN
EADHNGDLALDLALSRRLESIATTLVSHKADVDMVDKSGWSLLHKGIQRG
DLFAATFLIKNGAFVNAATLGAQETPLHLVALYSSKKHSADVMSEMAQIA
EALLQAGANPNMQDSKGRTPLHVSIMAGNEYVFSQLLQCKQLDLELKDHE
DSTALWLAVQHITVSSDQSVNPFEDVPVVNGTSFDENSFAARLIQRGSHT
DAPDTATGNCLLQRAAGAGNEAAALFLATNGAHVNHRNKWGETPLHTACR
HGLANLTAELLQQGANPNLQTEEALPLPKEAASLTSLADSVHLQTPLHMA
IAYNHPDVVSVILEQKANALHATNNLQIIPDFSLKDSRDQTVLGLALWTG
MHTIAAQLLGSGAAINDTMSDGQTLLHMAIQRQDSKSALFLLEHQADINV
RTQDGETALQLAIRNQLPLVVDAICTRGADMSVPDEKGNPPLWLALANNL
EDIASTLVRHGCDATCWGPGPGGCLQTLLHRAIDENNEPTACFLIRSGCD
VNSPRQPGANGEGEEEARDGQTPLHLAASWGLEETVQCLLEFGANVNAQD
AEGRTPIHVAISSQHGVIIQLLVSHPDIHLNVRDRQGLTPFACAMTFKNN
KSAEAILKRESGAAEQVDNKGRNFLHVAVQNSDIESVLFLISVHANVNSR
VQDASKLTPLHLAVQAGSEIIVRNLLLAGAKVNELTKHRQTALHLAAQQD
LPTICSVLLENGVDFAAVDENGNNALHLAVMHGRLNNIRVLLTECTVDAE
AFNLRGQSPLHILGQYGKENAAAIFDLFLECMPGYPLDKPDADGSTVLLL
AYMKGNANLCRAIVRSGARLGVNNNQGVNIFNYQVATKQLLFRLLDMLSK
EPPWCDGSYCYECTARFGVTTRKHHCRHCGRLLCHKCSTKEIPIIKFDLN
KPVRVCNICFDVLTLGGVS,

or an amino acid having at least 70%, 75%, 80%, 85%, 90%, 95%, or 99% sequence identity therewith, or fragment thereof.

Zinc finger protein: Q9P2G1-1:
(SEQ ID NO: 409)
MGNTTTKFRKALINGDENLACQIYENNPQLKESLDPNTSYGEPYQHNTPL
HYAARHGMNKILGTFLGRDGNPNKRNVHNETSMHLLCMGPQIMISEGALH
PRLARPTEDDFRRADCLQMILKWKGAKLDQGEYERAAIDAVDNKKNTPLH
YAAASGMKACVELLVKHGGDLFAENENKDTPCDCAEKQHHKDLALNLESQ
MVFSRDPEAEEIEAEYAALDKREPYEGLRPQDLRRLKDMLIVETADMLQA
PLFTAEALLRAHDWDREKLLEAWMSNPENCCQRSGVQMPTPPPSGYNAWD
TLPSPRTPRTTRSSVTSPDEISLSPGDLDTSLCDICMCSISVFEDPVDMP
CGHDFCRGCWESFLNLKIQEGEAHNIFCPAYDCFQLVPVDIIESVVSKEM
DKRYLQFDIKAFVENNPAIKWCPTPGCDRAVRLTKQGSNTSGSDTLSFPL
LRAPAVDCGKGHLFCWECLGEAHEPCDCQTWKNWLQKITEMKPEELVGVS
EAYEDAANCLWLLTNSKPCANCKSPIQKNEGCNHMQCAKCKYDFCWICLE
EWKKHSSSTGGYYRCTRYEVIQHVEEQSKEMTVEAEKKHKRFQELDRFMH
YYTRFKNHEHSYQLEQRLLKTAKEKMEQLSRALKETEGGCPDTTFIEDAV
HVLLKTRRILKCSYPYGFFLEPKSTKKEIFELMQTDLEMVTEDLAQKVNR
PYLRTPRHKIIKAACLVQQKRQEFLASVARGVAPADSPEAPRRSFAGGTW
DWEYLGFASPEEYAEFQYRRRHRQRRRGDVHSLLSNPPDPDEPSESTLDI
PEGGSSSRRPGTSVVSSASMSVLHSSSLRDYTPASRSENQDSLQALSSLD
EDDPNILLAIQLSLQESGLALDEETRDFLSNEASLGAIGTSLPSRLDSVP
RNTDSPRAALSSSELLELGDSLMRLGAENDPFSTDTLSSHPLSEARSDFC
PSSSDPDSAGQDPNINDNLLGNIMAWFHDMNPQSIALIPPATTEISADSQ
LPCIKDGSEGVKDVELVLPEDSMFEDASVSEGRGTQIEENPLEENILAGE
AASQAGDSGNEAANRGDGSDVSSQTPQTSSDWLEQVHLV,

or an amino acid having at least 70%, 75%, 80%, 85%, 90%, 95%, or 99% sequence identity therewith, or fragment thereof.

Zinc finger protein: Q8IUH5-1 (isoform 1):
(SEQ ID NO: 409)
MGNTTTKFRKALINGDENLACQIYENNPQLKESLDPNTSYGEPYQHNTPL
HYAARHGMNKILGTFLGRDGNPNKRNVHNETSMHLLCMGPQIMISEGALH
PRLARPTEDDFRRADCLQMILKWKGAKLDQGEYERAAIDAVDNKKNTPLH
YAAASGMKACVELLVKHGGDLFAENENKDTPCDCAEKQHHKDLALNLESQ
MVFSRDPEAEEIEAEYAALDKREPYEGLRPQDLRRLKDMLIVETADMLQA
PLFTAEALLRAHDWDREKLLEAWMSNPENCCQRSGVQMPTPPPSGYNAWD
TLPSPRTPRTTRSSVTSPDEISLSPGDLDTSLCDICMCSISVFEDPVDMP
CGHDFCRGCWESFLNLKIQEGEAHNIFCPAYDCFQLVPVDIIESVVSKEM
DKRYLQFDIKAFVENNPAIKWCPTPGCDRAVRLTKQGSNTSGSDTLSFPL
LRAPAVDCGKGHLFCWECLGEAHEPCDCQTWKNWLQKITEMKPEELVGVS
EAYEDAANCLWLLTNSKPCANCKSPIQKNEGCNHMQCAKCKYDFCWICLE
EWKKHSSSTGGYYRCTRYEVIQHVEEQSKEMTVEAEKKHKRFQELDRFMH
YYTRFKNHEHSYQLEQRLLKTAKEKMEQLSRALKETEGGCPDTTFIEDAV
HVLLKTRRILKCSYPYGFFLEPKSTKKEIFELMQTDLEMVTEDLAQKVNR
PYLRTPRHKIIKAACLVQQKRQEFLASVARGVAPADSPEAPRRSFAGGTW
DWEYLGFASPEEYAEFQYRRRHRQRRRGDVHSLLSNPPDPDEPSESTLDI
PEGGSSSRRPGTSVVSSASMSVLHSSSLRDYTPASRSENQDSLQALSSLD
EDDPNILLAIQLSLQESGLALDEETRDFLSNEASLGAIGTSLPSRLDSVP
RNTDSPRAALSSSELLELGDSLMRLGAENDPFSTDTLSSHPLSEARSDFC
PSSSDPDSAGQDPNINDNLLGNIMAWFHDMNPQSIALIPPATTEISADSQ
LPCIKDGSEGVKDVELVLPEDSMFEDASVSEGRGTQIEENPLEENILAGE
AASQAGDSGNEAANRGDGSDVSSQTPQTSSDWLEQVHLV,

or an amino acid having at least 70%, 75%, 80%, 85%, 90%, 95%, or 99% sequence identity therewith, or fragment thereof.

Zinc finger protein: P36406-1 (isoform alpha):
(SEQ ID NO: 410)
MATLVVNKLGAGVDSGRQGSRGTAVVKVLECGVCEDVFSLQGDKVPRLLL
CGHTVCHDCLTRLPLHGRAIRCPFDRQVTDLGDSGVWGLKKNFALLELLE
RLQNGPIGQYGAAEESIGISGESIIRCDEDEAHLASVYCTVCATHLCSEC
SQVTHSTKTLAKHRRVPLADKPHEKTMCSQHQVHAIEFVCLEEGCQTSPL
MCCVCKEYGKHQGHKHSVLEPEANQIRASILDMAHCIRTFTEEISDYSRK
LVGIVQHIEGGEQIVEDGIGMAHTEHVPGTAENARSCIRAYFYDLHETLC
RQEEMALSVVDAHVREKLIWLRQQQEDMTILLSEVSAACLHCEKTLQQDD
CRVVLAKQEITRLLETLQKQQQQFTEVADHIQLDASIPVTFTKDNRVHIG
PKMEIRVVTLGLDGAGKTTILFKLKQDEFMQPIPTIGFNVETVEYKNLKF
TIWDVGGKHKLRPLWKHYYLNTQAVVFVVDSSHRDRISEAHSELAKLLTE
KELRDALLLIFANKQDVAGALSVEEITELLSLHKLCCGRSWYIQGCDARS
GMGLYEGLDWLSRQLVAAGVLDVA,

or an amino acid having at least 70%, 75%, 80%, 85%, 90%, 95%, or 99% sequence identity therewith, or fragment thereof.

Zinc finger protein: Q9ULV3-1 (isoform-1):
(SEQ ID NO: 411)
MFSQQQQQQLQQQQQQLQQLQQQQLQQQQLQQQQLLQLQQLLQQSPPQAP
LPMAVSRGLPPQQPQQPLLNLQGTNSASLLNGSMLQRALLLQQLQGLDQF
AMPPATYDTAGLTMPTATLGNLRGYGMASPGLAAPSLTPPQLATPNLQQF
FPQATRQSLLGPPPVGVPMNPSQFNLSGRNPQKQARTSSSTTPNRKDSSS
QTMPVEDKSDPPEGSEEAAEPRMDTPEDQDLPPCPEDIAKEKRTPAPEPE
PCEASELPAKRLRSSEEPTEKEPPGQLQVKAQPQARMTVPKQTQTPDLLP
EALEAQVLPRFQPRVLQVQAQVQSQTQPRIPSTDTQVQPKLQKQAQTQTS
PEHLVLQQKQVQPQLQQEAEPQKQVQPQVQPQAHSQGPRQVQLQQEAEPL
KQVQPQVQPQAHSQPPRQVQLQLQKQVQTQTYPQVHTQAQPSVQPQEHPP
AQVSVQPPEQTHEQPHTQPQVSLLAPEQTPVVVHVCGLEMPPDAVEAGGG
MEKTLPEPVGTQVSMEEIQNESACGLDVGECENRAREMPGVWGAGGSLKV
TILQSSDSRAFSTVPLTPVPRPSDSVSSTPAATSTPSKQALQFFCYICKA
SCSSQQEFQDHMSEPQHQQRLGEIQHMSQACLLSLLPVPRDVLETEDEEP
PPRRWCNTCQLYYMGDLIQHRRTQDHKIAKQSLRPFCTVCNRYFKTPRKF
VEHVKSQGHKDKAKELKSLEKEIAGQDEDHFITVDAVGCFEGDEEEEEDD
EDEEEIEVEEELCKQVRSRDISREEWKGSETYSPNTAYGVDFLVPVMGYI
CRICHKFYHSNSGAQLSHCKSLGHFENLQKYKAAKNPSPTTRPVSRRCAI
NARNALTALFTSSGRPPSQPNTQDKTPSKVTARPSQPPLPRRSTRLKT,

or an amino acid having at least 70%, 75%, 80%, 85%, 90%, 95%, or 99% sequence identity therewith, or fragment thereof.

The present disclosure may use any known or available zinc finger protein, or variant or functional fragment thereof. In some embodiments, a mitoZF comprises an amino acid sequence selected from any one of the following amino acid sequences, or an amino acid sequence having at least 70%, 75%, 80%, 85%, 90%, 95%, or 99% sequence identity with any one of the following mitoZF sequences:

ZF (R8)
(SEQ ID NO: 224)
MAERPFQCDICMRNFSTSGSLSRHIRTHTGEKPFQCDICMRNFSQSGSLT
RHIRTHTGSEKPFQCDICMRNFSRSDALSQHIRTHTGEKPFQCDICMRNF
SRNDNRITHIRTHTGEKPFQCDICMRNFSRSDHLTQHTKIHLR
ZF (5xZnF-4-R8)
(SEQ ID NO: 225)
MAERPFQCDICMRNFSQASNLISHIRTHTGEKPFQCDICMRNFSTSHSLT
EHIRTHTGSEKPFQCDICMRNFSERSHLREHIRTHTGEKPFQCDICMRNF
SQSGNLTEHIRTHTGEKPFQCDICMRNFSSKKALTEHTKIHLR
ZF (5xZnF-10-R8)
(SEQ ID NO: 226)
MAERPFQCDICMRNFSQASNLISHIRTHTGEKPFQCDICMRNFSQRANLR
AHIRTHTGSEKPFQCDICMRNFSQASNLISHIRTHTGEKPFQCDICMRNF
STSHSLTEHIRTHTGEKPFQCDICMRNFSERSHLREHTKIHLR
ZF (R13-1)
(SEQ ID NO: 227)
MAERPFQCDICMRNFSRSDNLSTHIRTHTGEKPFQCDICMRNFSDRSDLS
RHIRTHTGEKPFQCDICMRNFSQSGDLTRHIRTHTGSEKPFQCDICMRNF
SRSDSLSAHIRTHTGEKPFQCDICMRNFSQKATRITHTKIHLR
ZF (5xZnF-9-R13)
(SEQ ID NO: 228)
MAERPFQCDICMRNFSQSSSLVRHIRTHTGEKPFQCDICMRNFSRSDNLV
RHIRTHTGSEKPFQCDICMRNFSQAGHLASHIRTHTGEKPFQCDICMRNF
SRKDNLKNHIRTHTGEKPFQCDICMRNFSRKDALRGHTKIHLR
ZF (5xZnF-12-R13)
(SEQ ID NO: 229)
MAERPFQCDICMRNFSRSDHLTTHIRTHTGEKPFQCDICMRNFSQSSSLV
RHIRTHTGSEKPFQCDICMRNFSRSDNLVRHIRTHTGEKPFQCDICMRNF
SQAGHLASHIRTHTGEKPFQCDICMRNFSRKDNLKNHTKIHLR

### NapDNAbp

In various embodiments, the Evolved DddA-containing base editors or the polypeptides that comprise the Evolved DddA-containing base editors (e.g., the pDNAbps and DddA) may include a napDNAbp as the pDNAbp component.

In one aspect, the methods and base editor compositions described herein involve a nucleic acid programmable DNA binding protein (napDNAbp). Each napDNAbp is associated with at least one guide nucleic acid (e.g., guide RNA), which localizes the napDNAbp to a DNA sequence that comprises a DNA strand (i.e., a target strand) that is complementary to the guide nucleic acid, or a portion thereof (e.g., the protospacer of a guide RNA). In other words, the guide nucleic-acid “programs” the napDNAbp (e.g., Cas9 or equivalent) to localize and bind to a complementary sequence. In various embodiments, the napDNAbp can be fused to a herein disclosed adenosine deaminase or cytidine deaminase.

Without being bound by theory, the binding mechanism of a napDNAbp-guide RNA complex, in general, includes the step of forming an R-loop whereby the napDNAbp induces the unwinding of a double-strand DNA target, thereby separating the strands in the region bound by the napDNAbp. The guideRNA protospacer then hybridizes to the “target strand.” This displaces a “non-target strand” that is complementary to the target strand, which forms the single strand region of the R-loop. In some embodiments, the napDNAbp includes one or more nuclease activities, which then cut the DNA leaving various types of lesions. For example, the napDNAbp may comprises a nuclease activity that cuts the non-target strand at a first location, and/or cuts the target strand at a second location. Depending on the nuclease activity, the target DNA can be cut to form a “double-stranded break” whereby both strands are cut. In other embodiments, the target DNA can be cut at only a single site, i.e., the DNA is “nicked” on one strand. Exemplary napDNAbp with different nuclease activities include “Cas9 nickase” (“nCas9”) and a deactivated Cas9 having no nuclease activities (“dead Cas9” or “dCas9”).

The below description of various napDNAbps which can be used in connection with the presently disclose base editors is not meant to be limiting in any way. The base editors may comprise the canonical SpCas9, or any ortholog Cas9 protein, or any variant Cas9 protein—including any naturally occurring variant, mutant, or otherwise engineered version of Cas9—that is known or which can be made or evolved through a directed evolutionary or otherwise mutagenic process. In various embodiments, the Cas9 or Cas9 variants have a nickase activity, i.e., only cleave of strand of the target DNA sequence. In other embodiments, the Cas9 or Cas9 variants have inactive nucleases, i.e., are “dead” Cas9 proteins. Other variant Cas9 proteins that may be used are those having a smaller molecular weight than the canonical SpCas9 (e.g., for easier delivery) or having modified or rearranged primary amino acid structure (e.g., the circular permutant formats). The base editors described herein may also comprise Cas9 equivalents, including Cas12a/Cpf1 and Cas12b proteins which are the result of convergent evolution. The napDNAbps used herein (e.g., SpCas9, Cas9 variant, or Cas9 equivalents) may also contain various modifications that alter/enhance their PAM specifies. Lastly, the application contemplates any Cas9, Cas9 variant, or Cas9 equivalent which has at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity to a reference Cas9 sequence, such as a references SpCas9 canonical sequence or a reference Cas9 equivalent (e.g., Cas12a/Cpf1).

The napDNAbp can be a CRISPR (clustered regularly interspaced short palindromic repeat)-associated nuclease. As outlined above, CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek M. et al., Science 337:816-821(2012), the entire contents of which is hereby incorporated by reference.

In some embodiments, the napDNAbp directs cleavage of one or both strands at the location of a target sequence, such as within the target sequence and/or within the complement of the target sequence. In some embodiments, the napDNAbp directs cleavage of one or both strands within about 1, 2, 3, 4, 5, 6, 7, 8, 9, 10, 15, 20, 25, 50,100,200, 500, or more base pairs from the first or last nucleotide of a target sequence. In some embodiments, a vector encodes a napDNAbp that is mutated to with respect to a corresponding wild-type enzyme such that the mutated napDNAbp lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A in reference to the canonical SpCas9 sequence, or to equivalent amino acid positions in other Cas9 variants or Cas9 equivalents.

As used herein, the term “Cas protein” refers to a full-length Cas protein obtained from nature, a recombinant Cas protein having a sequences that differs from a naturally occurring Cas protein, or any fragment of a Cas protein that nevertheless retains all or a significant amount of the requisite basic functions needed for the disclosed methods, i.e., (i) possession of nucleic-acid programmable binding of the Cas protein to a target DNA, and (ii) ability to nick the target DNA sequence on one strand. The Cas proteins contemplated herein embrace CRISPR Cas 9 proteins, as well as Cas9 equivalents, variants (e.g., Cas9 nickase (nCas9) or nuclease inactive Cas9 (dCas9)) homologs, orthologs, or paralogs, whether naturally occurring or non-naturally occurring (e.g., engineered or recombinant), and may include a Cas9 equivalent from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353(6299), the contents of which are incorporated herein by reference.

The terms “Cas9” or “Cas9 nuclease” or “Cas9 moiety” or “Cas9 domain” embrace any naturally occurring Cas9 from any organism, any naturally-occurring Cas9 equivalent or functional fragment thereof, any Cas9 homolog, ortholog, or paralog from any organism, and any mutant or variant of a Cas9, naturally-occurring or engineered. The term Cas9 is not meant to be particularly limiting and may be referred to as a “Cas9 or equivalent.” Exemplary Cas9 proteins are further described herein and/or are described in the art and are incorporated herein by reference. The present disclosure is unlimited with regard to the particular Cas9 that is employed in the base editor (PE) of the invention.

As noted herein, Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference).

Examples of Cas9 and Cas9 equivalents are provided as follows; however, these specific examples are not meant to be limiting. The base editor fusions of the present disclosure may use any suitable napDNAbp, including any suitable Cas9 or Cas9 equivalent.

### (1) Wild Type SpCas9

In one embodiment, the base editor constructs described herein may comprise the “canonical SpCas9” nuclease from S. pyogenes, which has been widely used as a tool for genome engineering. This Cas9 protein is a large, multi-domain protein containing two distinct nuclease domains. Point mutations can be introduced into Cas9 to abolish one or both nuclease activities, resulting in a nickase Cas9 (nCas9) or dead Cas9 (dCas9), respectively, that still retains its ability to bind DNA in a sgRNA-programmed manner. In principle, when fused to another protein or domain, Cas9 or variant thereof (e.g., nCas9) can target that protein to virtually any DNA sequence simply by co-expression with an appropriate sgRNA. As used herein, the canonical SpCas9 protein refers to the wild type protein from Streptococcus pyogenes having the following amino acid sequence:

| Description | Sequence | SEQ ID NO: |
|---|---|---|
| SpCas9 | MDKKYSIGLDIGTNSVGWAVITDEYKVPSKKFKVLGNTDRHSIKKNLIGALLFDS | 59 |
| Streptococcus | GETAEATRLKRTARRRYTRRKNRICYLQEIFSNEMAKVDDSFFHRLEESFLVEED | |
| pyogenes M1 | KKHERHPIFGNIVDEVAYHEKYPTIYHLRKKLVDSTDKADLRLIYLALAHMIKFR | |
| SwissProt | GHFLIEGDLNPDNSDVDKLFIQLVQTYNQLFEENPINASGVDAKAILSARLSKSRR | |
| Accession No. | LENLIAQLPGEKKNGLFGNLIALSLGLTPNFKSNFDLAEDAKLQLSKDTYDDDLD | |
| Q99ZW2 | NLLAQIGDQYADLFLAAKNLSDAILLSDILRVNTEITKAPLSASMIKRYDEHHQDL | |
| Wild type | TLLKALVRQQLPEKYKEIFFDQSKNGYAGYIDGGASQEEFYKFIKPILEKMDGTE | |
| | ELLVKLNREDLLRKQRTFDNGSIPHQIHLGELHAILRRQEDFYPFLKDNREKIEKIL | |
| | TFRIPYYVGPLARGNSRFAWMTRKSEETITPWNFEEVVDKGASAQSFIERMTNFD | |
| | KNLPNEKVLPKHSLLYEYFTVYNELTKVKYVTEGMRKPAFLSGEQKKAIVDLLF | |
| | KTNRKVTVKQLKEDYFKKIECFDSVEISGVEDRFNASLGTYHDLLKIIKDKDFLD | |
| | NEENEDILEDIVLTLTLFEDREMIEERLKTYAHLFDDKVMKQLKRRRYTGWGRL | |
| | SRKLINGIRDKQSGKTILDFLKSDGFANRNFMQLIHDDSLTFKEDIQKAQVSGQG | |
| | DSLHEHIANLAGSPAIKKGILQTVKVVDELVKVMGRHKPENIVIEMARENQTTQK | |
| | GQKNSRERMKRIEEGIKELGSQILKEHPVENTQLQNEKLYLYYLQNGRDMYVDQ | |
| | ELDINRLSDYDVDHIVPQSFLKDDSIDNKVLTRSDKNRGKSDNVPSEEVVKKMK | |
| | NYWRQLLNAKLITQRKFDNLTKAERGGLSELDKAGFIKRQLVETRQITKHVAQIL | |
| | DSRMNTKYDENDKLIREVKVITLKSKLVSDFRKDFQFYKVREINNYHHAHDAYL | |
| | NAVVGTALIKKYPKLESEFVYGDYKVYDVRKMIAKSEQEIGKATAKYFFYSNIM | |
| | NFFKTEITLANGEIRKRPLIETNGETGEIVWDKGRDFATVRKVLSMPQVNIVKKTE | |
| | VQTGGFSKESILPKRNSDKLIARKKDWDPKKYGGFDSPTVAYSVLVVAKVEKGK | |
| | SKKLKSVKELLGITIMERSSFEKNPIDFLEAKGYKEVKKDLIIKLPKYSLFELENGR | |
| | KRMLASAGELQKGNELALPSKYVNFLYLASHYEKLKGSPEDNEQKQLFVEQHK | |
| | HYLDEIIEQISEFSKRVILADANLDKVLSAYNKHRDKPIREQAENIIHLFTLTNLGA | |
| | PAAFKYFDTTIDRKRYTSTKEVLDATLIHQSITGLYETRIDLSQLGGD | |
| SpCas9 | ATGGATAAAAAATATAGCATTGGCCTGGATATTGGCACCAACAGCGTGGGCT | 60 |
| Reverse | GGGCGGTGATTACCGATGAATATAAAGTGCCGAGCAAAAAATTTAAAGTGCT | |
| translation of | GGGCAACACCGATCGCCATAGCATTAAAAAAAACCTGATTGGCGCGCTGCTG | |
| SwissProt | TTTGATAGCGGCGAAACCGCGGAAGCGACCCGCCTGAAACGCACCGCGCGCC | |
| Accession No. | GCCGCTATACCCGCCGCAAAAACCGCATTTGCTATCTGCAGGAAATTTTTAGC | |
| Q99ZW2 | AACGAAATGGCGAAAGTGGATGATAGCTTTTTTCATCGCCTGGAAGAAAGCT | |
| Streptococcus | TTCTGGTGGAAGAAGATAAAAAACATGAACGCCATCCGATTTTTGGCAACAT | |
| pyogenes | TGTGGATGAAGTGGCGTATCATGAAAAATATCCGACCATTTATCATCTGCGC | |
| | AAAAAACTGGTGGATAGCACCGATAAAGCGGATCTGCGCCTGATTTATCTGG | |
| | CGCTGGCGCATATGATTAAATTTCGCGGCCATTTTCTGATTGAAGGCGATCTG | |
| | AACCCGGATAACAGCGATGTGGATAAACTGTTTATTCAGCTGGTGCAGACCT | |
| | ATAACCAGCTGTTTGAAGAAAACCCGATTAACGCGAGCGGCGTGGATGCGAA | |
| | AGCGATTCTGAGCGCGCGCCTGAGCAAAAGCCGCCGCCTGGAAAACCTGATT | |
| | GCGCAGCTGCCGGGCGAAAAAAAAAACGGCCTGTTTGGCAACCTGATTGCGC | |
| | TGAGCCTGGGCCTGACCCCGAACTTTAAAAGCAACTTTGATCTGGCGGAAGA | |
| | TGCGAAACTGCAGCTGAGCAAAGATACCTATGATGATGATCTGGATAACCTG | |
| | CTGGCGCAGATTGGCGATCAGTATGCGGATCTGTTTCTGGCGGCGAAAAACC | |
| | TGAGCGATGCGATTCTGCTGAGCGATATTCTGCGCGTGAACACCGAAATTAC | |
| | CAAAGCGCCGCTGAGCGCGAGCATGATTAAACGCTATGATGAACATCATCAG | |
| | GATCTGACCCTGCTGAAAGCGCTGGTGCGCCAGCAGCTGCCGGAAAAATATA | |
| | AAGAAATTTTTTTTGATCAGAGCAAAAACGGCTATGCGGGCTATATTGATGG | |
| | CGGCGCGAGCCAGGAAGAATTTTATAAATTTATTAAACCGATTCTGGAAAAA | |
| | ATGGATGGCACCGAAGAACTGCTGGTGAAACTGAACCGCGAAGATCTGCTGC | |
| | GCAAACAGCGCACCTTTGATAACGGCAGCATTCCGCATCAGATTCATCTGGG | |
| | CGAACTGCATGCGATTCTGCGCCGCCAGGAAGATTTTTATCCGTTTCTGAAAG | |
| | ATAACCGCGAAAAAATTGAAAAAATTCTGACCTTTCGCATTCCGTATTATGTG | |
| | GGCCCGCTGGCGCGCGGCAACAGCCGCTTTGCGTGGATGACCCGCAAAAGCG | |
| | AAGAAACCATTACCCCGTGGAACTTTGAAGAAGTGGTGGATAAAGGCGCGA | |
| | GCGCGCAGAGCTTTATTGAACGCATGACCAACTTTGATAAAAACCTGCCGAA | |
| | CGAAAAAGTGCTGCCGAAACATAGCCTGCTGTATGAATATTTTACCGTGTAT | |
| | AACGAACTGACCAAAGTGAAATATGTGACCGAAGGCATGCGCAAACCGGCG | |
| | TTTCTGAGCGGCGAACAGAAAAAAGCGATTGTGGATCTGCTGTTTAAAACCA | |
| | ACCGCAAAGTGACCGTGAAACAGCTGAAAGAAGATTATTTTAAAAAAATTGA | |
| | ATGCTTTGATAGCGTGGAAATTAGCGGCGTGGAAGATCGCTTTAACGCGAGC | |
| | CTGGGCACCTATCATGATCTGCTGAAAATTATTAAAGATAAAGATTTTCTGGA | |
| | TAACGAAGAAAACGAAGATATTCTGGAAGATATTGTGCTGACCCTGACCCTG | |
| | TTTGAAGATCGCGAAATGATTGAAGAACGCCTGAAAACCTATGCGCATCTGT | |
| | TTGATGATAAAGTGATGAAACAGCTGAAACGCCGCCGCTATACCGGCTGGGG | |
| | CCGCCTGAGCCGCAAACTGATTAACGGCATTCGCGATAAACAGAGCGGCAAA | |
| | ACCATTCTGGATTTTCTGAAAAGCGATGGCTTTGCGAACCGCAACTTTATGCA | |
| | GCTGATTCATGATGATAGCCTGACCTTTAAAGAAGATATTCAGAAAGCGCAG | |
| | GTGAGCGGCCAGGGCGATAGCCTGCATGAACATATTGCGAACCTGGCGGGCA | |
| | GCCCGGCGATTAAAAAAGGCATTCTGCAGACCGTGAAAGTGGTGGATGAACT | |
| | GGTGAAAGTGATGGGCCGCCATAAACCGGAAAACATTGTGATTGAAATGGCG | |
| | CGCGAAAACCAGACCACCCAGAAAGGCCAGAAAAACAGCCGCGAACGCATG | |
| | AAACGCATTGAAGAAGGCATTAAAGAACTGGGCAGCCAGATTCTGAAAGAA | |
| | CATCCGGTGGAAAACACCCAGCTGCAGAACGAAAAACTGTATCTGTATTATC | |
| | TGCAGAACGGCCGCGATATGTATGTGGATCAGGAACTGGATATTAACCGCCT | |
| | GAGCGATTATGATGTGGATCATATTGTGCCGCAGAGCTTTCTGAAAGATGAT | |
| | AGCATTGATAACAAAGTGCTGACCCGCAGCGATAAAAACCGCGGCAAAAGC | |
| | GATAACGTGCCGAGCGAAGAAGTGGTGAAAAAAATGAAAAACTATTGGCGC | |
| | CAGCTGCTGAACGCGAAACTGATTACCCAGCGCAAATTTGATAACCTGACCA | |
| | AAGCGGAACGCGGCGGCCTGAGCGAACTGGATAAAGCGGGCTTTATTAAAC | |
| | GCCAGCTGGTGGAAACCCGCCAGATTACCAAACATGTGGCGCAGATTCTGGA | |
| | TAGCCGCATGAACACCAAATATGATGAAAACGATAAACTGATTCGCGAAGTG | |
| | AAAGTGATTACCCTGAAAAGCAAACTGGTGAGCGATTTTCGCAAAGATTTTC | |
| | AGTTTTATAAAGTGCGCGAAATTAACAACTATCATCATGCGCATGATGCGTAT | |
| | CTGAACGCGGTGGTGGGCACCGCGCTGATTAAAAAATATCCGAAACTGGAAA | |
| | GCGAATTTGTGTATGGCGATTATAAAGTGTATGATGTGCGCAAAATGATTGC | |
| | GAAAAGCGAACAGGAAATTGGCAAAGCGACCGCGAAATATTTTTTTTATAGC | |
| | AACATTATGAACTTTTTTAAAACCGAAATTACCCTGGCGAACGGCGAAATTC | |
| | GCAAACGCCCGCTGATTGAAACCAACGGCGAAACCGGCGAAATTGTGTGGG | |
| | ATAAAGGCCGCGATTTTGCGACCGTGCGCAAAGTGCTGAGCATGCCGCAGGT | |
| | GAACATTGTGAAAAAAACCGAAGTGCAGACCGGCGGCTTTAGCAAAGAAAG | |
| | CATTCTGCCGAAACGCAACAGCGATAAACTGATTGCGCGCAAAAAAGATTGG | |
| | GATCCGAAAAAATATGGCGGCTTTGATAGCCCGACCGTGGCGTATAGCGTGC | |
| | TGGTGGTGGCGAAAGTGGAAAAAGGCAAAAGCAAAAAACTGAAAAGCGTGA | |
| | AAGAACTGCTGGGCATTACCATTATGGAACGCAGCAGCTTTGAAAAAAACCC | |
| | GATTGATTTTCTGGAAGCGAAAGGCTATAAAGAAGTGAAAAAAGATCTGATT | |
| | ATTAAACTGCCGAAATATAGCCTGTTTGAACTGGAAAACGGCCGCAAACGCA | |
| | TGCTGGCGAGCGCGGGCGAACTGCAGAAAGGCAACGAACTGGCGCTGCCGA | |
| | GCAAATATGTGAACTTTCTGTATCTGGCGAGCCATTATGAAAAACTGAAAGG | |
| | CAGCCCGGAAGATAACGAACAGAAACAGCTGTTTGTGGAACAGCATAAACA | |
| | TTATCTGGATGAAATTATTGAACAGATTAGCGAATTTAGCAAACGCGTGATTC | |
| | TGGCGGATGCGAACCTGGATAAAGTGCTGAGCGCGTATAACAAACATCGCGA | |
| | TAAACCGATTCGCGAACAGGCGGAAAACATTATTCATCTGTTTACCCTGACC | |
| | AACCTGGGCGCGCCGGCGGCGTTTAAATATTTTGATACCACCATTGATCGCA | |
| | AACGCTATACCAGCACCAAAGAAGTGCTGGATGCGACCCTGATTCATCAGAG | |
| | CATTACCGGCCTGTATGAAACCCGCATTGATCTGAGCCAGCTGGGCGGCGAT | |

The base editors described herein may include canonical SpCas9, or any variant thereof having at least 80%, at least 85%, at least 90%, at least 95%, or at least 99% sequence identity with a wild type Cas9 sequence provided above. These variants may include SpCas9 variants containing one or more mutations, including any known mutation reported with the SwissProt Accession No. Q99ZW2 entry, which include:

| SpCas9 mutation (relative | Function/Characteristic |
|---|---|
| to the amino acid sequence | (as reported) (see UniProtKB- |
| of the canonical SpCas9 | Q99ZW2 (CAS9_STRPT1) entry- |
| sequence, SEQ ID NO: 59) | incorporated herein by reference) |
| D10A | Nickase mutant which cleaves the |
| | protospacer strand (but no cleavage |
| | of non-protospacer strand) |
| S15A | Decreased DNA cleavage activity |
| R66A | Decreased DNA cleavage activity |
| R70A | No DNA cleavage |
| R74A | Decreased DNA cleavage |
| R78A | Decreased DNA cleavage |
| 97-150 deletion | No nuclease activity |
| R165A | Decreased DNA cleavage |
| 175-307 deletion | About 50% decreased DNA cleavage |
| 312-409 deletion | No nuclease activity |
| E762A | Nickase |
| H840A | Nickase mutant which cleaves the non- |
| | protospacer strand but does not cleave |
| | the protospacer strand |
| N854A | Nickase |
| N863A | Nickase |
| H982A | Decreased DNA cleavage |
| D986A | Nickase |
| 1099-1368 deletion | No nuclease activity |
| R1333A | Reduced DNA binding |

Other wild type SpCas9 sequences that may be used in the present disclosure, include:

| Description | Sequence | SEQ ID NO: |
|---|---|---|
| SpCas9 | ATGGATAAGAAATACTCAATAGGCTTAGATATCGGCACAAATAGCGTCGG | 61 |
| Streptococcus | ATGGGCGGTGATCACTGATGATTATAAGGTTCCGTCTAAAAAGTTCAAGGT | |
| pyogenes | TCTGGGAAATACAGACCGCCACAGTATCAAAAAAAATCTTATAGGGGCTCT | |
| MGAS1882 | TTTATTTGGCAGTGGAGAGACAGCGGAAGCGACTCGTCTCAAACGGACAGC | |
| wild type | TCGTAGAAGGTATACACGTCGGAAGAATCGTATTTGTTATCTACAGGAGAT | |
| NC_017053.1 | TTTTTCAAATGAGATGGCGAAAGTAGATGATAGTTTCTTTCATCGACTTGAA | |
| | GAGTCTTTTTTGGTGGAAGAAGACAAGAAGCATGAACGTCATCCTATTTTT | |
| | GGAAATATAGTAGATGAAGTTGCTTATCATGAGAAATATCCAACTATCTAT | |
| | CATCTGCGAAAAAAATTGGCAGATTCTACTGATAAAGCGGATTTGCGCTTA | |
| | ATCTATTTGGCCTTAGCGCATATGATTAAGTTTCGTGGTCATTTTTTGATTG | |
| | AGGGAGATTTAAATCCTGATAATAGTGATGTGGACAAACTATTTATCCAGT | |
| | TGGTACAAATCTACAATCAATTATTTGAAGAAAACCCTATTAACGCAAGTA | |
| | GAGTAGATGCTAAAGCGATTCTTTCTGCACGATTGAGTAAATCAAGACGAT | |
| | TAGAAAATCTCATTGCTCAGCTCCCCGGTGAGAAGAGAAATGGCTTGTTTG | |
| | GGAATCTCATTGCTTTGTCATTGGGATTGACCCCTAATTTTAAATCAAATTT | |
| | TGATTTGGCAGAAGATGCTAAATTACAGCTTTCAAAAGATACTTACGATGA | |
| | TGATTTAGATAATTTATTGGCGCAAATTGGAGATCAATATGCTGATTTGTTT | |
| | TTGGCAGCTAAGAATTTATCAGATGCTATTTTACTTTCAGATATCCTAAGAG | |
| | TAAATAGTGAAATAACTAAGGCTCCCCTATCAGCTTCAATGATTAAGCGCT | |
| | ACGATGAACATCATCAAGACTTGACTCTTTTAAAAGCTTTAGTTCGACAAC | |
| | AACTTCCAGAAAAGTATAAAGAAATCTTTTTTGATCAATCAAAAAACGGAT | |
| | ATGCAGGTTATATTGATGGGGGAGCTAGCCAAGAAGAATTTTATAAATTTA | |
| | TCAAACCAATTTTAGAAAAAATGGATGGTACTGAGGAATTATTGGTGAAAC | |
| | TAAATCGTGAAGATTTGCTGCGCAAGCAACGGACCTTTGACAACGGCTCTA | |
| | TTCCCCATCAAATTCACTTGGGTGAGCTGCATGCTATTTTGAGAAGACAAG | |
| | AAGACTTTTATCCATTTTTAAAAGACAATCGTGAGAAGATTGAAAAAATCT | |
| | TGACTTTTCGAATTCCTTATTATGTTGGTCCATTGGCGCGTGGCAATAGTCG | |
| | TTTTGCATGGATGACTCGGAAGTCTGAAGAAACAATTACCCCATGGAATTT | |
| | TGAAGAAGTTGTCGATAAAGGTGCTTCAGCTCAATCATTTATTGAACGCAT | |
| | GACAAACTTTGATAAAAATCTTCCAAATGAAAAAGTACTACCAAAACATAG | |
| | TTTGCTTTATGAGTATTTTACGGTTTATAACGAATTGACAAAGGTCAAATAT | |
| | GTTACTGAGGGAATGCGAAAACCAGCATTTCTTTCAGGTGAACAGAAGAA | |
| | AGCCATTGTTGATTTACTCTTCAAAACAAATCGAAAAGTAACCGTTAAGCA | |
| | ATTAAAAGAAGATTATTTCAAAAAAATAGAATGTTTTGATAGTGTTGAAAT | |
| | TTCAGGAGTTGAAGATAGATTTAATGCTTCATTAGGCGCCTACCATGATTTG | |
| | CTAAAAATTATTAAAGATAAAGATTTTTTGGATAATGAAGAAAATGAAGAT | |
| | ATCTTAGAGGATATTGTTTTAACATTGACCTTATTTGAAGATAGGGGGATG | |
| | ATTGAGGAAAGACTTAAAACATATGCTCACCTCTTTGATGATAAGGTGATG | |
| | AAACAGCTTAAACGTCGCCGTTATACTGGTTGGGGACGTTTGTCTCGAAAA | |
| | TTGATTAATGGTATTAGGGATAAGCAATCTGGCAAAACAATATTAGATTTT | |
| | TTGAAATCAGATGGTTTTGCCAATCGCAATTTTATGCAGCTGATCCATGATG | |
| | ATAGTTTGACATTTAAAGAAGATATTCAAAAAGCACAGGTGTCTGGACAAG | |
| | GCCATAGTTTACATGAACAGATTGCTAACTTAGCTGGCAGTCCTGCTATTA | |
| | AAAAAGGTATTTTACAGACTGTAAAAATTGTTGATGAACTGGTCAAAGTAA | |
| | TGGGGCATAAGCCAGAAAATATCGTTATTGAAATGGCACGTGAAAATCAG | |
| | ACAACTCAAAAGGGCCAGAAAAATTCGCGAGAGCGTATGAAACGAATCGA | |
| | AGAAGGTATCAAAGAATTAGGAAGTCAGATTCTTAAAGAGCATCCTGTTGA | |
| | AAATACTCAATTGCAAAATGAAAAGCTCTATCTCTATTATCTACAAAATGG | |
| | AAGAGACATGTATGTGGACCAAGAATTAGATATTAATCGTTTAAGTGATTA | |
| | TGATGTCGATCACATTGTTCCACAAAGTTTCATTAAAGACGATTCAATAGA | |
| | CAATAAGGTACTAACGCGTTCTGATAAAAATCGTGGTAAATCGGATAACGT | |
| | TCCAAGTGAAGAAGTAGTCAAAAAGATGAAAAACTATTGGAGACAACTTC | |
| | TAAACGCCAAGTTAATCACTCAACGTAAGTTTGATAATTTAACGAAAGCTG | |
| | AACGTGGAGGTTTGAGTGAACTTGATAAAGCTGGTTTTATCAAACGCCAAT | |
| | TGGTTGAAACTCGCCAAATCACTAAGCATGTGGCACAAATTTTGGATAGTC | |
| | GCATGAATACTAAATACGATGAAAATGATAAACTTATTCGAGAGGTTAAAG | |
| | TGATTACCTTAAAATCTAAATTAGTTTCTGACTTCCGAAAAGATTTCCAATT | |
| | CTATAAAGTACGTGAGATTAACAATTACCATCATGCCCATGATGCGTATCT | |
| | AAATGCCGTCGTTGGAACTGCTTTGATTAAGAAATATCCAAAACTTGAATC | |
| | GGAGTTTGTCTATGGTGATTATAAAGTTTATGATGTTCGTAAAATGATTGCT | |
| | AAGTCTGAGCAAGAAATAGGCAAAGCAACCGCAAAATATTTCTTTTACTCT | |
| | AATATCATGAACTTCTTCAAAACAGAAATTACACTTGCAAATGGAGAGATT | |
| | CGCAAACGCCCTCTAATCGAAACTAATGGGGAAACTGGAGAAATTGTCTGG | |
| | GATAAAGGGCGAGATTTTGCCACAGTGCGCAAAGTATTGTCCATGCCCCAA | |
| | GTCAATATTGTCAAGAAAACAGAAGTACAGACAGGCGGATTCTCCAAGGA | |
| | GTCAATTTTACCAAAAAGAAATTCGGACAAGCTTATTGCTCGTAAAAAAGA | |
| | CTGGGATCCAAAAAAATATGGTGGTTTTGATAGTCCAACGGTAGCTTATTC | |
| | AGTCCTAGTGGTTGCTAAGGTGGAAAAAGGGAAATCGAAGAAGTTAAAAT | |
| | CCGTTAAAGAGTTACTAGGGATCACAATTATGGAAAGAAGTTCCTTTGAAA | |
| | AAAATCCGATTGACTTTTTAGAAGCTAAAGGATATAAGGAAGTTAAAAAA | |
| | GACTTAATCATTAAACTACCTAAATATAGTCTTTTTGAGTTAGAAAACGGTC | |
| | GTAAACGGATGCTGGCTAGTGCCGGAGAATTACAAAAAGGAAATGAGCTG | |
| | GCTCTGCCAAGCAAATATGTGAATTTTTTATATTTAGCTAGTCATTATGAAA | |
| | AGTTGAAGGGTAGTCCAGAAGATAACGAACAAAAACAATTGTTTGTGGAG | |
| | CAGCATAAGCATTATTTAGATGAGATTATTGAGCAAATCAGTGAATTTTCT | |
| | AAGCGTGTTATTTTAGCAGATGCCAATTTAGATAAAGTTCTTAGTGCATATA | |
| | ACAAACATAGAGACAAACCAATACGTGAACAAGCAGAAAATATTATTCAT | |
| | TTATTTACGTTGACGAATCTTGGAGCTCCCGCTGCTTTTAAATATTTTGATA | |
| | CAACAATTGATCGTAAACGATATACGTCTACAAAAGAAGTTTTAGATGCCA | |
| | CTCTTATCCATCAATCCATCACTGGTCTTTATGAAACACGCATTGATTTGAG | |
| | TCAGCTAGGAGGTGACTGA | |
| SpCas9 | MDKKYSIGLDIGTNSVGWAVITDDYKVPSKKFKVLGNTDRHSIKKNLIGALLF | 62 |
| Streptococcus | GSGETAEATRLKRTARRRYTRRKNRICYLQEIFSNEMAKVDDSFFHRLEESFLV | |
| pyogenes | EEDKKHERHPIFGNIVDEVAYHEKYPTIYHLRKKLADSTDKADLRLIYLALAH | |
| MGAS1882 | MIKFRGHFLIEGDLNPDNSDVDKLFIQLVQIYNQLFEENPINASRVDAKAILSAR | |
| wild type | LSKSRRLENLIAQLPGEKRNGLFGNLIALSLGLTPNFKSNFDLAEDAKLQLSKD | |
| NC_017053.1 | TYDDDLDNLLAQIGDQYADLFLAAKNLSDAILLSDILRVNSEITKAPLSASMIK | |
| | RYDEHHQDLTLLKALVRQQLPEKYKEIFFDQSKNGYAGYIDGGASQEEFYKFI | |
| | KPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDK | |
| | GASAQSFIERMTNFDKNLPNEKVLPKHSLLYEYFTVYNELTKVKYVTEGMRKP | |
| | AFLSGEQKKAIVDLLFKTNRKVTVKQLKEDYFKKIECFDSVEISGVEDRFNASL | |
| | GAYHDLLKIIKDKDFLDNEENEDILEDIVLTLTLFEDRGMIEERLKTYAHLFDD | |
| | KVMKQLKRRRYTGWGRLSRKLINGIRDKQSGKTILDFLKSDGFANRNFMQLIH | |
| | DDSLTFKEDIQKAQVSGQGHSLHEQIANLAGSPAIKKGILQTVKIVDELVKVMG | |
| | HKPENIVIEMARENQTTQKGQKNSRERMKRIEEGIKELGSQILKEHPVENTQLQ | |
| | NEKLYLYYLQNGRDMYVDQELDINRLSDYDVDHIVPQSFIKDDSIDNKVLTRS | |
| | DKNRGKSDNVPSEEVVKKMKNYWRQLLNAKLITQRKFDNLTKAERGGLSEL | |
| | DKAGFIKRQLVETRQITKHVAQILDSRMNTKYDENDKLIREVKVITLKSKLVSD | |
| | FRKDFQFYKVREINNYHHAHDAYLNAVVGTALIKKYPKLESEFVYGDYKVYD | |
| | VRKMIAKSEQEIGKATAKYFFYSNIMNFFKTEITLANGEIRKRPLIETNGETGEI | |
| | VWDKGRDFATVRKVLSMPQVNIVKKTEVQTGGFSKESILPKRNSDKLIARKKD | |
| | WDPKKYGGFDSPTVAYSVLVVAKVEKGKSKKLKSVKELLGITIMERSSFEKNP | |
| | IDFLEAKGYKEVKKDLIIKLPKYSLFELENGRKRMLASAGELQKGNELALPSKY | |
| | VNFLYLASHYEKLKGSPEDNEQKQLFVEQHKHYLDEIIEQISEFSKRVILADAN | |
| | LDKVLSAYNKHRDKPIREQAENIIHLFTLTNLGAPAAFKYFDTTIDRKRYTSTK | |
| | EVLDATLIHQSITGLYETRIDLSQLGGD | |
| SpCas9 | ATGGATAAAAAGTATTCTATTGGTTTAGACATCGGCACTAATTCCGTTGGA | 63 |
| Streptococcus | TGGGCTGTCATAACCGATGAATACAAAGTACCTTCAAAGAAATTTAAGGTG | |
| pyogenes | TTGGGGAACACAGACCGTCATTCGATTAAAAAGAATCTTATCGGTGCCCTC | |
| wild type | CTATTCGATAGTGGCGAAACGGCAGAGGCGACTCGCCTGAAACGAACCGC | |
| SWBC2D7W | TCGGAGAAGGTATACACGTCGCAAGAACCGAATATGTTACTTACAAGAAAT | |
| 014 | TTTTAGCAATGAGATGGCCAAAGTTGACGATTCTTTCTTTCACCGTTTGGAA | |
| | GAGTCCTTCCTTGTCGAAGAGGACAAGAAACATGAACGGCACCCCATCTTT | |
| | GGAAACATAGTAGATGAGGTGGCATATCATGAAAAGTACCCAACGATTTAT | |
| | CACCTCAGAAAAAAGCTAGTTGACTCAACTGATAAAGCGGACCTGAGGTTA | |
| | ATCTACTTGGCTCTTGCCCATATGATAAAGTTCCGTGGGCACTTTCTCATTG | |
| | AGGGTGATCTAAATCCGGACAACTCGGATGTCGACAAACTGTTCATCCAGT | |
| | TAGTACAAACCTATAATCAGTTGTTTGAAGAGAACCCTATAAATGCAAGTG | |
| | GCGTGGATGCGAAGGCTATTCTTAGCGCCCGCCTCTCTAAATCCCGACGGC | |
| | TAGAAAACCTGATCGCACAATTACCCGGAGAGAAGAAAAATGGGTTGTTC | |
| | GGTAACCTTATAGCGCTCTCACTAGGCCTGACACCAAATTTTAAGTCGAAC | |
| | TTCGACTTAGCTGAAGATGCCAAATTGCAGCTTAGTAAGGACACGTACGAT | |
| | GACGATCTCGACAATCTACTGGCACAAATTGGAGATCAGTATGCGGACTTA | |
| | TTTTTGGCTGCCAAAAACCTTAGCGATGCAATCCTCCTATCTGACATACTGA | |
| | GAGTTAATACTGAGATTACCAAGGCGCCGTTATCCGCTTCAATGATCAAAA | |
| | GGTACGATGAACATCACCAAGACTTGACACTTCTCAAGGCCCTAGTCCGTC | |
| | AGCAACTGCCTGAGAAATATAAGGAAATATTCTTTGATCAGTCGAAAAACG | |
| | GGTACGCAGGTTATATTGACGGCGGAGCGAGTCAAGAGGAATTCTACAAG | |
| | TTTATCAAACCCATATTAGAGAAGATGGATGGGACGGAAGAGTTGCTTGTA | |
| | AAACTCAATCGCGAAGATCTACTGCGAAAGCAGCGGACTTTCGACAACGGT | |
| | AGCATTCCACATCAAATCCACTTAGGCGAATTGCATGCTATACTTAGAAGG | |
| | CAGGAGGATTTTTATCCGTTCCTCAAAGACAATCGTGAAAAGATTGAGAAA | |
| | ATCCTAACCTTTCGCATACCTTACTATGTGGGACCCCTGGCCCGAGGGAAC | |
| | TCTCGGTTCGCATGGATGACAAGAAAGTCCGAAGAAACGATTACTCCATGG | |
| | AATTTTGAGGAAGTTGTCGATAAAGGTGCGTCAGCTCAATCGTTCATCGAG | |
| | AGGATGACCAACTTTGACAAGAATTTACCGAACGAAAAAGTATTGCCTAAG | |
| | CACAGTTTACTTTACGAGTATTTCACAGTGTACAATGAACTCACGAAAGTT | |
| | AAGTATGTCACTGAGGGCATGCGTAAACCCGCCTTTCTAAGCGGAGAACAG | |
| | AAGAAAGCAATAGTAGATCTGTTATTCAAGACCAACCGCAAAGTGACAGTT | |
| | AAGCAATTGAAAGAGGACTACTTTAAGAAAATTGAATGCTTCGATTCTGTC | |
| | GAGATCTCCGGGGTAGAAGATCGATTTAATGCGTCACTTGGTACGTATCAT | |
| | GACCTCCTAAAGATAATTAAAGATAAGGACTTCCTGGATAACGAAGAGAA | |
| | TGAAGATATCTTAGAAGATATAGTGTTGACTCTTACCCTCTTTGAAGATCGG | |
| | GAAATGATTGAGGAAAGACTAAAAACATACGCTCACCTGTTCGACGATAA | |
| | GGTTATGAAACAGTTAAAGAGGCGTCGCTATACGGGCTGGGGACGATTGTC | |
| | GCGGAAACTTATCAACGGGATAAGAGACAAGCAAAGTGGTAAAACTATTC | |
| | TCGATTTTCTAAAGAGCGACGGCTTCGCCAATAGGAACTTTATGCAGCTGA | |
| | TCCATGATGACTCTTTAACCTTCAAAGAGGATATACAAAAGGCACAGGTTT | |
| | CCGGACAAGGGGACTCATTGCACGAACATATTGCGAATCTTGCTGGTTCGC | |
| | CAGCCATCAAAAAGGGCATACTCCAGACAGTCAAAGTAGTGGATGAGCTA | |
| | GTTAAGGTCATGGGACGTCACAAACCGGAAAACATTGTAATCGAGATGGC | |
| | ACGCGAAAATCAAACGACTCAGAAGGGGCAAAAAAACAGTCGAGAGCGG | |
| | ATGAAGAGAATAGAAGAGGGTATTAAAGAACTGGGCAGCCAGATCTTAAA | |
| | GGAGCATCCTGTGGAAAATACCCAATTGCAGAACGAGAAACTTTACCTCTA | |
| | TTACCTACAAAATGGAAGGGACATGTATGTTGATCAGGAACTGGACATAAA | |
| | CCGTTTATCTGATTACGACGTCGATCACATTGTACCCCAATCCTTTTTGAAG | |
| | GACGATTCAATCGACAATAAAGTGCTTACACGCTCGGATAAGAACCGAGG | |
| | GAAAAGTGACAATGTTCCAAGCGAGGAAGTCGTAAAGAAAATGAAGAACT | |
| | ATTGGCGGCAGCTCCTAAATGCGAAACTGATAACGCAAAGAAAGTTCGAT | |
| | AACTTAACTAAAGCTGAGAGGGGTGGCTTGTCTGAACTTGACAAGGCCGGA | |
| | TTTATTAAACGTCAGCTCGTGGAAACCCGCCAAATCACAAAGCATGTTGCA | |
| | CAGATACTAGATTCCCGAATGAATACGAAATACGACGAGAACGATAAGCT | |
| | GATTCGGGAAGTCAAAGTAATCACTTTAAAGTCAAAATTGGTGTCGGACTT | |
| | CAGAAAGGATTTTCAATTCTATAAAGTTAGGGAGATAAATAACTACCACCA | |
| | TGCGCACGACGCTTATCTTAATGCCGTCGTAGGGACCGCACTCATTAAGAA | |
| | ATACCCGAAGCTAGAAAGTGAGTTTGTGTATGGTGATTACAAAGTTTATGA | |
| | CGTCCGTAAGATGATCGCGAAAAGCGAACAGGAGATAGGCAAGGCTACAG | |
| | CCAAATACTTCTTTTATTCTAACATTATGAATTTCTTTAAGACGGAAATCAC | |
| | TCTGGCAAACGGAGAGATACGCAAACGACCTTTAATTGAAACCAATGGGG | |
| | AGACAGGTGAAATCGTATGGGATAAGGGCCGGGACTTCGCGACGGTGAGA | |
| | AAAGTTTTGTCCATGCCCCAAGTCAACATAGTAAAGAAAACTGAGGTGCAG | |
| | ACCGGAGGGTTTTCAAAGGAATCGATTCTTCCAAAAAGGAATAGTGATAAG | |
| | CTCATCGCTCGTAAAAAGGACTGGGACCCGAAAAAGTACGGTGGCTTCGAT | |
| | AGCCCTACAGTTGCCTATTCTGTCCTAGTAGTGGCAAAAGTTGAGAAGGGA | |
| | AAATCCAAGAAACTGAAGTCAGTCAAAGAATTATTGGGGATAACGATTAT | |
| | GGAGCGCTCGTCTTTTGAAAAGAACCCCATCGACTTCCTTGAGGCGAAAGG | |
| | TTACAAGGAAGTAAAAAAGGATCTCATAATTAAACTACCAAAGTATAGTCT | |
| | GTTTGAGTTAGAAAATGGCCGAAAACGGATGTTGGCTAGCGCCGGAGAGC | |
| | TTCAAAAGGGGAACGAACTCGCACTACCGTCTAAATACGTGAATTTCCTGT | |
| | ATTTAGCGTCCCATTACGAGAAGTTGAAAGGTTCACCTGAAGATAACGAAC | |
| | AGAAGCAACTTTTTGTTGAGCAGCACAAACATTATCTCGACGAAATCATAG | |
| | AGCAAATTTCGGAATTCAGTAAGAGAGTCATCCTAGCTGATGCCAATCTGG | |
| | ACAAAGTATTAAGCGCATACAACAAGCACAGGGATAAACCCATACGTGAG | |
| | CAGGCGGAAAATATTATCCATTTGTTTACTCTTACCAACCTCGGCGCTCCAG | |
| | CCGCATTCAAGTATTTTGACACAACGATAGATCGCAAACGATACACTTCTA | |
| | CCAAGGAGGTGCTAGACGCGACACTGATTCACCAATCCATCACGGGATTAT | |
| | ATGAAACTCGGATAGATTTGTCACAGCTTGGGGGTGACGGATCCCCCAAGA | |
| | AGAAGAGGAAAGTCTCGAGCGACTACAAAGACCATGACGGTGATTATAAA | |
| | GATCATGACATCGATTACAAGGATGACGATGACAAGGCTGCAGGA | |
| SpCas9 | MDKKYSIGLDIGTNSVGWAVITDEYKVPSKKFKVLGNTDRHSIKKNLIGALLF | 64 |
| Streptococcus | DSGETAEATRLKRTARRRYTRRKNRICYLQEIFSNEMAKVDDSFFHRLEESFLV | |
| pyogenes | EEDKKHERHPIFGNIVDEVAYHEKYPTIYHLRKKLVDSTDKADLRLIYLALAH | |
| wild type | MIKFRGHFLIEGDLNPDNSDVDKLFIQLVQTYNQLFEENPINASGVDAKAILSA | |
| Encoded | RLSKSRRLENLIAQLPGEKKNGLFGNLIALSLGLTPNFKSNFDLAEDAKLQLSK | |
| product of | DTYDDDLDNLLAQIGDQYADLFLAAKNLSDAILLSDILRVNTEITKAPLSASMI | |
| SWBC2D7W014 | KRYDEHHQDLTLLKALVRQQLPEKYKEIFFDQSKNGYAGYIDGGASQEEFYKF | |
| | IKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDK | |
| | GASAQSFIERMTNFDKNLPNEKVLPKHSLLYEYFTVYNELTKVKYVTEGMRKP | |
| | AFLSGEQKKAIVDLLFKTNRKVTVKQLKEDYFKKIECFDSVEISGVEDRFNASL | |
| | GTYHDLLKIIKDKDFLDNEENEDILEDIVLTLTLFEDREMIEERLKTYAHLFDDK | |
| | VMKQLKRRRYTGWGRLSRKLINGIRDKQSGKTILDFLKSDGFANRNFMQLIHD | |
| | DSLTFKEDIQKAQVSGQGDSLHEHIANLAGSPAIKKGILQTVKVVDELVKVMG | |
| | RHKPENIVIEMARENQTTQKGQKNSRERMKRIEEGIKELGSQILKEHPVENTQL | |
| | QNEKLYLYYLQNGRDMYVDQELDINRLSDYDVDHIVPQSFLKDDSIDNKVLT | |
| | RSDKNRGKSDNVPSEEVVKKMKNYWRQLLNAKLITQRKFDNLTKAERGGLSE | |
| | LDKAGFIKRQLVETRQITKHVAQILDSRMNTKYDENDKLIREVKVITLKSKLVS | |
| | DFRKDFQFYKVREINNYHHAHDAYLNAVVGTALIKKYPKLESEFVYGDYKVY | |
| | DVRKMIAKSEQEIGKATAKYFFYSNIMNFFKTEITLANGEIRKRPLIETNGETGE | |
| | IVWDKGRDFATVRKVLSMPQVNIVKKTEVQTGGFSKESILPKRNSDKLIARKK | |
| | DWDPKKYGGFDSPTVAYSVLVVAKVEKGKSKKLKSVKELLGITIMERSSFEKN | |
| | PIDFLEAKGYKEVKKDLIIKLPKYSLFELENGRKRMLASAGELQKGNELALPSK | |
| | YVNFLYLASHYEKLKGSPEDNEQKQLFVEQHKHYLDEIIEQISEFSKRVILADA | |
| | NLDKVLSAYNKHRDKPIREQAENIIHLFTLTNLGAPAAFKYFDTTIDRKRYTST | |
| | KEVLDATLIHQSITGLYETRIDLSQLGGDGSPKKKRKVSSDYKDHDGDYKDHD | |
| | IDYKDDDDKAAG | |
| SpCas9 | ATGGATAAGAAATACTCAATAGGCTTAGATATCGGCACAAATAGCGTCGG | 65 |
| Streptococcus | ATGGGCGGTGATCACTGATGAATATAAGGTTCCGTCTAAAAAGTTCAAGGT | |
| pyogenes | TCTGGGAAATACAGACCGCCACAGTATCAAAAAAAATCTTATAGGGGCTCT | |
| M1GAS wild | TTTATTTGACAGTGGAGAGACAGCGGAAGCGACTCGTCTCAAACGGACAGC | |
| type | TCGTAGAAGGTATACACGTCGGAAGAATCGTATTTGTTATCTACAGGAGAT | |
| NC_002737.2 | TTTTTCAAATGAGATGGCGAAAGTAGATGATAGTTTCTTTCATCGACTTGAA | |
| | GAGTCTTTTTTGGTGGAAGAAGACAAGAAGCATGAACGTCATCCTATTTTT | |
| | GGAAATATAGTAGATGAAGTTGCTTATCATGAGAAATATCCAACTATCTAT | |
| | CATCTGCGAAAAAAATTGGTAGATTCTACTGATAAAGCGGATTTGCGCTTA | |
| | ATCTATTTGGCCTTAGCGCATATGATTAAGTTTCGTGGTCATTTTTTGATTG | |
| | AGGGAGATTTAAATCCTGATAATAGTGATGTGGACAAACTATTTATCCAGT | |
| | TGGTACAAACCTACAATCAATTATTTGAAGAAAACCCTATTAACGCAAGTG | |
| | GAGTAGATGCTAAAGCGATTCTTTCTGCACGATTGAGTAAATCAAGACGAT | |
| | TAGAAAATCTCATTGCTCAGCTCCCCGGTGAGAAGAAAAATGGCTTATTTG | |
| | GGAATCTCATTGCTTTGTCATTGGGTTTGACCCCTAATTTTAAATCAAATTT | |
| | TGATTTGGCAGAAGATGCTAAATTACAGCTTTCAAAAGATACTTACGATGA | |
| | TGATTTAGATAATTTATTGGCGCAAATTGGAGATCAATATGCTGATTTGTTT | |
| | TTGGCAGCTAAGAATTTATCAGATGCTATTTTACTTTCAGATATCCTAAGAG | |
| | TAAATACTGAAATAACTAAGGCTCCCCTATCAGCTTCAATGATTAAACGCT | |
| | ACGATGAACATCATCAAGACTTGACTCTTTTAAAAGCTTTAGTTCGACAAC | |
| | AACTTCCAGAAAAGTATAAAGAAATCTTTTTTGATCAATCAAAAAACGGAT | |
| | ATGCAGGTTATATTGATGGGGGAGCTAGCCAAGAAGAATTTTATAAATTTA | |
| | TCAAACCAATTTTAGAAAAAATGGATGGTACTGAGGAATTATTGGTGAAAC | |
| | TAAATCGTGAAGATTTGCTGCGCAAGCAACGGACCTTTGACAACGGCTCTA | |
| | TTCCCCATCAAATTCACTTGGGTGAGCTGCATGCTATTTTGAGAAGACAAG | |
| | AAGACTTTTATCCATTTTTAAAAGACAATCGTGAGAAGATTGAAAAAATCT | |
| | TGACTTTTCGAATTCCTTATTATGTTGGTCCATTGGCGCGTGGCAATAGTCG | |
| | TTTTGCATGGATGACTCGGAAGTCTGAAGAAACAATTACCCCATGGAATTT | |
| | TGAAGAAGTTGTCGATAAAGGTGCTTCAGCTCAATCATTTATTGAACGCAT | |
| | GACAAACTTTGATAAAAATCTTCCAAATGAAAAAGTACTACCAAAACATAG | |
| | TTTGCTTTATGAGTATTTTACGGTTTATAACGAATTGACAAAGGTCAAATAT | |
| | GTTACTGAAGGAATGCGAAAACCAGCATTTCTTTCAGGTGAACAGAAGAA | |
| | AGCCATTGTTGATTTACTCTTCAAAACAAATCGAAAAGTAACCGTTAAGCA | |
| | ATTAAAAGAAGATTATTTCAAAAAAATAGAATGTTTTGATAGTGTTGAAAT | |
| | TTCAGGAGTTGAAGATAGATTTAATGCTTCATTAGGTACCTACCATGATTTG | |
| | CTAAAAATTATTAAAGATAAAGATTTTTTGGATAATGAAGAAAATGAAGAT | |
| | ATCTTAGAGGATATTGTTTTAACATTGACCTTATTTGAAGATAGGGAGATG | |
| | ATTGAGGAAAGACTTAAAACATATGCTCACCTCTTTGATGATAAGGTGATG | |
| | AAACAGCTTAAACGTCGCCGTTATACTGGTTGGGGACGTTTGTCTCGAAAA | |
| | TTGATTAATGGTATTAGGGATAAGCAATCTGGCAAAACAATATTAGATTTT | |
| | TTGAAATCAGATGGTTTTGCCAATCGCAATTTTATGCAGCTGATCCATGATG | |
| | ATAGTTTGACATTTAAAGAAGACATTCAAAAAGCACAAGTGTCTGGACAAG | |
| | GCGATAGTTTACATGAACATATTGCAAATTTAGCTGGTAGCCCTGCTATTA | |
| | AAAAAGGTATTTTACAGACTGTAAAAGTTGTTGATGAATTGGTCAAAGTAA | |
| | TGGGGCGGCATAAGCCAGAAAATATCGTTATTGAAATGGCACGTGAAAAT | |
| | CAGACAACTCAAAAGGGCCAGAAAAATTCGCGAGAGCGTATGAAACGAAT | |
| | CGAAGAAGGTATCAAAGAATTAGGAAGTCAGATTCTTAAAGAGCATCCTGT | |
| | TGAAAATACTCAATTGCAAAATGAAAAGCTCTATCTCTATTATCTCCAAAA | |
| | TGGAAGAGACATGTATGTGGACCAAGAATTAGATATTAATCGTTTAAGTGA | |
| | TTATGATGTCGATCACATTGTTCCACAAAGTTTCCTTAAAGACGATTCAATA | |
| | GACAATAAGGTCTTAACGCGTTCTGATAAAAATCGTGGTAAATCGGATAAC | |
| | GTTCCAAGTGAAGAAGTAGTCAAAAAGATGAAAAACTATTGGAGACAACT | |
| | TCTAAACGCCAAGTTAATCACTCAACGTAAGTTTGATAATTTAACGAAAGC | |
| | TGAACGTGGAGGTTTGAGTGAACTTGATAAAGCTGGTTTTATCAAACGCCA | |
| | ATTGGTTGAAACTCGCCAAATCACTAAGCATGTGGCACAAATTTTGGATAG | |
| | TCGCATGAATACTAAATACGATGAAAATGATAAACTTATTCGAGAGGTTAA | |
| | AGTGATTACCTTAAAATCTAAATTAGTTTCTGACTTCCGAAAAGATTTCCAA | |
| | TTCTATAAAGTACGTGAGATTAACAATTACCATCATGCCCATGATGCGTAT | |
| | CTAAATGCCGTCGTTGGAACTGCTTTGATTAAGAAATATCCAAAACTTGAA | |
| | TCGGAGTTTGTCTATGGTGATTATAAAGTTTATGATGTTCGTAAAATGATTG | |
| | CTAAGTCTGAGCAAGAAATAGGCAAAGCAACCGCAAAATATTTCTTTTACT | |
| | CTAATATCATGAACTTCTTCAAAACAGAAATTACACTTGCAAATGGAGAGA | |
| | TTCGCAAACGCCCTCTAATCGAAACTAATGGGGAAACTGGAGAAATTGTCT | |
| | GGGATAAAGGGCGAGATTTTGCCACAGTGCGCAAAGTATTGTCCATGCCCC | |
| | AAGTCAATATTGTCAAGAAAACAGAAGTACAGACAGGCGGATTCTCCAAG | |
| | GAGTCAATTTTACCAAAAAGAAATTCGGACAAGCTTATTGCTCGTAAAAAA | |
| | GACTGGGATCCAAAAAAATATGGTGGTTTTGATAGTCCAACGGTAGCTTAT | |
| | TCAGTCCTAGTGGTTGCTAAGGTGGAAAAAGGGAAATCGAAGAAGTTAAA | |
| | ATCCGTTAAAGAGTTACTAGGGATCACAATTATGGAAAGAAGTTCCTTTGA | |
| | AAAAAATCCGATTGACTTTTTAGAAGCTAAAGGATATAAGGAAGTTAAAA | |
| | AAGACTTAATCATTAAACTACCTAAATATAGTCTTTTTGAGTTAGAAAACG | |
| | GTCGTAAACGGATGCTGGCTAGTGCCGGAGAATTACAAAAAGGAAATGAG | |
| | CTGGCTCTGCCAAGCAAATATGTGAATTTTTTATATTTAGCTAGTCATTATG | |
| | AAAAGTTGAAGGGTAGTCCAGAAGATAACGAACAAAAACAATTGTTTGTG | |
| | GAGCAGCATAAGCATTATTTAGATGAGATTATTGAGCAAATCAGTGAATTT | |
| | TCTAAGCGTGTTATTTTAGCAGATGCCAATTTAGATAAAGTTCTTAGTGCAT | |
| | ATAACAAACATAGAGACAAACCAATACGTGAACAAGCAGAAAATATTATT | |
| | CATTTATTTACGTTGACGAATCTTGGAGCTCCCGCTGCTTTTAAATATTTTG | |
| | ATACAACAATTGATCGTAAACGATATACGTCTACAAAAGAAGTTTTAGATG | |
| | CCACTCTTATCCATCAATCCATCACTGGTCTTTATGAAACACGCATTGATTT | |
| | GAGTCAGCTAGGAGGTGACTGA | |
| SpCas9 | MDKKYSIGLDIGTNSVGWAVITDEYKVPSKKFKVLGNTDRHSIKKNLIGALLF | 59 |
| Streptococcus | DSGETAEATRLKRTARRRYTRRKNRICYLQEIFSNEMAKVDDSFFHRLEESFLV | |
| pyogenes | EEDKKHERHPIFGNIVDEVAYHEKYPTIYHLRKKLVDSTDKADLRLIYLALAH | |
| MGAS wild | MIKFRGHFLIEGDLNPDNSDVDKLFIQLVQTYNQLFEENPINASGVDAKAILSA | |
| type | RLSKSRRLENLIAQLPGEKKNGLFGNLIALSLGLTPNFKSNFDLAEDAKLQLSK | |
| Encoded | DTYDDDLDNLLAQIGDQYADLFLAAKNLSDAILLSDILRVNTEITKAPLSASMI | |
| product of | KRYDEHHQDLTLLKALVRQQLPEKYKEIFFDQSKNGYAGYIDGGASQEEFYKF | |
| NC_002737.2 | IKPILEKMDGTEELLVKLNREDLLRKQRTFDNGSIPHQIHLGELHAILRRQEDFY | |
| (100% | PFLKDNREKIEKILTFRIPYYVGPLARGNSRFAWMTRKSEETITPWNFEEVVDK | |
| identical to | GASAQSFIERMTNFDKNLPNEKVLPKHSLLYEYFTVYNELTKVKYVTEGMRKP | |
| the canonical | AFLSGEQKKAIVDLLFKTNRKVTVKQLKEDYFKKIECFDSVEISGVEDRFNASL | |
| Q99ZW2 | GTYHDLLKIIKDKDFLDNEENEDILEDIVLTLTLFEDREMIEERLKTYAHLFDDK | |
| wild type) | VMKQLKRRRYTGWGRLSRKLINGIRDKQSGKTILDFLKSDGFANRNFMQLIHD | |
| | DSLTFKEDIQKAQVSGQGDSLHEHIANLAGSPAIKKGILQTVKVVDELVKVMG | |
| | RHKPENIVIEMARENQTTQKGQKNSRERMKRIEEGIKELGSQILKEHPVENTQL | |
| | QNEKLYLYYLQNGRDMYVDQELDINRLSDYDVDHIVPQSFLKDDSIDNKVLT | |
| | RSDKNRGKSDNVPSEEVVKKMKNYWRQLLNAKLITQRKFDNLTKAERGGLSE | |
| | LDKAGFIKRQLVETRQITKHVAQILDSRMNTKYDENDKLIREVKVITLKSKLVS | |
| | DFRKDFQFYKVREINNYHHAHDAYLNAVVGTALIKKYPKLESEFVYGDYKVY | |
| | DVRKMIAKSEQEIGKATAKYFFYSNIMNFFKTEITLANGEIRKRPLIETNGETGE | |
| | IVWDKGRDFATVRKVLSMPQVNIVKKTEVQTGGFSKESILPKRNSDKLIARKK | |
| | DWDPKKYGGFDSPTVAYSVLVVAKVEKGKSKKLKSVKELLGITIMERSSFEKN | |
| | PIDFLEAKGYKEVKKDLIIKLPKYSLFELENGRKRMLASAGELQKGNELALPSK | |
| | YVNFLYLASHYEKLKGSPEDNEQKQLFVEQHKHYLDEIIEQISEFSKRVILADA | |
| | NLDKVLSAYNKHRDKPIREQAENIIHLFTLTNLGAPAAFKYFDTTIDRKRYTST | |
| | KEVLDATLIHQSITGLYETRIDLSQLGGD | |

The base editors described herein may include any of the above SpCas9 sequences, or any variant thereof having at least 80%, at least 85%, at least 90%, at least 95%, or at least 99% sequence identity thereto.

### (2) Wild Type Cas9 Orthologs

In other embodiments, the Cas9 protein can be a wild type Cas9 ortholog from another bacterial species. For example, the following Cas9 orthologs can be used in connection with the base editor constructs described in this specification. In addition, any variant Cas9 orthologs having at least 80%, at least 85%, at least 90%, at least 95%, or at least 99% sequence identity to any of the below orthologs may also be used with the present base editors.

| Description | Sequence |
|---|---|
| LfCas9 | MKEYHIGLDIGTSSIGWAVTDSQFKLMRIKGKTAIGVRLFEEGKTAAERRTFRTTRRRLKR |
| Lactobacillus | RKWRLHYLDEIFAPHLQEVDENFLRRLKQSNIHPEDPTKNQAFIGKLLFPDLLKKNERGYP |
| fermentum wild | TLIKMRDELPVEQRAHYPVMNIYKLREAMINEDRQFDLREVYLAVHHIVKYRGHFLNNA |
| type | SVDKFKVGRIDFDKSFNVLNEAYEELQNGEGSFTIEPSKVEKIGQLLLDTKMRKLDRQKA |
| GenBank: | VAKLLEVKVADKEETKRNKQIATAMSKLVLGYKADFATVAMANGNEWKIDLSSETSED |
| SNX31424.11 | EIEKFREELSDAQNDILTEITSLFSQIMLNEIVPNGMSISESMMDRYWTHERQLAEVKEYLA |
| | TQPASARKEFDQVYNKYIGQAPKERGFDLEKGLKKILSKKENWKEIDELLKAGDFLPKQR |
| | TSANGVIPHQMHQQELDRIIEKQAKYYPWLATENPATGERDRHQAKYELDQLVSFRIPYY |
| | VGPLVTPEVQKATSGAKFAWAKRKEDGEITPWNLWDKIDRAESAEAFIKRMTVKDTYLL |
| | NEDVLPANSLLYQKYNVLNELNNVRVNGRRLSVGIKQDIYTELFKKKKTVKASDVASLV |
| | MAKTRGVNKPSVEGLSDPKKFNSNLATYLDLKSIVGDKVDDNRYQTDLENIIEWRSVFED |
| | GEIFADKLTEVEWLTDEQRSALVKKRYKGWGRLSKKLLTGIVDENGQRIIDLMWNTDQN |
| | FKEIVDQPVFKEQIDQLNQKAITNDGMTLRERVESVLDDAYTSPQNKKAIWQVVRVVEDI |
| | VKAVGNAPKSISIEFARNEGNKGEITRSRRTQLQKLFEDQAHELVKDTSLTEELEKAPDLS |
| | DRYYFYFTQGGKDMYTGDPINFDEISTKYDIDHILPQSFVKDNSLDNRVLTSRKENNKKS |
| | DQVPAKLYAAKMKPYWNQLLKQGLITQRKFENLTKDVDQNIKYRSLGFVKRQLVETRQ |
| | VIKLTANILGSMYQEAGTEIIETRAGLTKQLREEFDLPKVREVNDYHHAVDAYLTTFAGQ |
| | YLNRRYPKLRSFFVYGEYMKFKHGSDLKLRNFNFFHELMEGDKSQGKVVDQQTGELITT |
| | RDEVAKSFDRLLNMKYMLVSKEVHDRSDQLYGATIVTAKESGKLTSPIEIKKNRLVDLYG |
| | AYTNGTSAFMTIIKFTGNKPKYKVIGIPTTSAASLKRAGKPGSESYNQELHRIIKSNPKVKK |
| | GFEIVVPHVSYGQLIVDGDCKFTLASPTVQHPATQLVLSKKSLETISSGYKILKDKPAIANE |
| | RLIRVFDEVVGQMNRYFTIFDQRSNRQKVADARDKFLSLPTESKYEGAKKVQVGKTEVIT |
| | NLLMGLHANATQGDLKVLGLATFGFFQSTTGLSLSEDTMIVYQSPTGLFERRICLKDI |
| | (SEQ ID NO: 66) |
| SaCas9 | MDKKYSIGLDIGTNSVGWAVITDEYKVPSKKFKVLGNTDRHSIKKNLIGALLFDSGETAE |
| Staphylococcus | ATRLKRTARRRYTRRKNRICYLQEIFSNEMAKVDDSFFHRLEESFLVEEDKKHERHPIFGN |
| aureus wild type | IVDEVAYHEKYPTIYHLRKKLVDSTDKADLRLIYLALAHMIKFRGHFLIEGDLNPDNSDVD |
| GenBank: | KLFIQLVQTYNQLFEENPINASGVDAKAILSARLSKSRRLENLIAQLPGEKKNGLFGNLIAL |
| AYD60528.1 | SLGLTPNFKSNFDLAEDAKLQLSKDTYDDDLDNLLAQIGDQYADLFLAAKNLSDAILLSDI |
| | LRVNTEITKAPLSASMIKRYDEHHQDLTLLKALVRQQLPEKYKEIFFDQSKNGYAGYIDG |
| | GASQEEFYKFIKPILEKMDGTEELLVKLNREDLLRKQRTFDNGSIPHQIHLGELHAILRRQE |
| | DFYPFLKDNREKIEKILTFRIPYYVGPLARGNSRFAWMTRKSEETITPWNFEEVVDKGASA |
| | QSFIERMTNFDKNLPNEKVLPKHSLLYEYFTVYNELTKVKYVTEGMRKPAFLSGEQKKAI |
| | VDLLFKTNRKVTVKQLKEDYFKKIECFDSVEISGVEDRFNASLGTYHDLLKIIKDKDFLDN |
| | EENEDILEDIVLTLTLFEDREMIEERLKTYAHLFDDKVMKQLKRRRYTGWGRLSRKLINGI |
| | RDKQSGKTILDFLKSDGFANRNFMQLIHDDSLTFKEDIQKAQVSGQGDSLHEHIANLAGSP |
| | AIKKGILQTVKVVDELVKVMGRHKPENIVIEMARENQTTQKGQKNSRERMKRIEEGIKEL |
| | GSQILKEHPVENTQLQNEKLYLYYLQNGRDMYVDQELDINRLSDYDVDHIVPQSFLKDDS |
| | IDNKVLTRSDKNRGKSDNVPSEEVVKKMKNYWRQLLNAKLITQRKFDNLTKAERGGLSE |
| | LDKAGFIKRQLVETRQITKHVAQILDSRMNTKYDENDKLIREVKVITLKSKLVSDFRKDFQ |
| | FYKVREINNYHHAHDAYLNAVVGTALIKKYPKLESEFVYGDYKVYDVRKMIAKSEQEIG |
| | KATAKYFFYSNIMNFFKTEITLANGEIRKRPLIETNGETGEIVWDKGRDFATVRKVLSMPQ |
| | VNIVKKTEVQTGGFSKESILPKRNSDKLIARKKDWDPKKYGGFDSPTVAYSVLVVAKVEK |
| | GKSKKLKSVKELLGITIMERSSFEKNPIDFLEAKGYKEVKKDLIIKLPKYSLFELENGRKRM |
| | LASAGELQKGNELALPSKYVNFLYLASHYEKLKGSPEDNEQKQLFVEQHKHYLDEIIEQIS |
| | EFSKRVILADANLDKVLSAYNKHRDKPIREQAENIIHLFTLTNLGAPAAFKYFDTTIDRKR |
| | YTSTKEVLDATLIHQSITGLYETRIDLSQLGGD (SEQ ID NO: 59) |
| SaCas9 | MGKRNYILGLDIGITSVGYGIIDYETRDVIDAGVRLFKEANVENNEGRRSKRGARRLKRR |
| Staphylococcus | RRHRIQRVKKLLFDYNLLTDHSELSGINPYEARVKGLSQKLSEEEFSAALLHLAKRRGVH |
| aureus | NVNEVEEDTGNELSTKEQISRNSKALEEKYVAELQLERLKKDGEVRGSINRFKTSDYVKE |
| | AKQLLKVQKAYHQLDQSFIDTYIDLLETRRTYYEGPGEGSPFGWKDIKEWYEMLMGHCT |
| | YFPEELRSVKYAYNADLYNALNDLNNLVITRDENEKLEYYEKFQIIENVFKQKKKPTLKQI |
| | AKEILVNEEDIKGYRVTSTGKPEFTNLKVYHDIKDITARKEIIENAELLDQIAKILTIYQSSE |
| | DIQEELTNLNSELTQEEIEQISNLKGYTGTHNLSLKAINLILDELWHTNDNQIAIFNRLKLVP |
| | KKVDLSQQKEIPTTLVDDFILSPVVKRSFIQSIKVINAIIKKYGLPNDIIIELAREKNSKDAQK |
| | MINEMQKRNRQTNERIEEIIRTTGKENAKYLIEKIKLHDMQEGKCLYSLEAIPLEDLLNNPF |
| | NYEVDHIIPRSVSFDNSFNNKVLVKQEENSKKGNRTPFQYLSSSDSKISYETFKKHILNLAK |
| | GKGRISKTKKEYLLEERDINRFSVQKDFINRNLVDTRYATRGLMNLLRSYFRVNNLDVKV |
| | KSINGGFTSFLRRKWKFKKERNKGYKHHAEDALIIANADFIFKEWKKLDKAKKVMENQM |
| | FEEKQAESMPEIETEQEYKEIFITPHQIKHIKDFKDYKYSHRVDKKPNRKLINDTLYSTRKD |
| | DKGNTLIVNNLNGLYDKDNDKLKKLINKSPEKLLMYHHDPQTYQKLKLIMEQYGDEKNP |
| | LYKYYEETGNYLTKYSKKDNGPVIKKIKYYGNKLNAHLDITDDYPNSRNKVVKLSLKPY |
| | RFDVYLDNGVYKFVTVKNLDVIKKENYYEVNSKCYEEAKKLKKISNQAEFIASFYKNDLI |
| | KINGELYRVIGVNNDLLNRIEVNMIDITYREYLENMNDKRPPHIIKTIASKTQSIKKYSTDIL |
| | GNLYEVKSKKHPQIIKK |
| | (SEQ ID NO: 67) |
| StCas9 | MLFNKCIIISINLDFSNKEKCMTKPYSIGLDIGTNSVGWAVITDNYKVPSKKMKVLGNTSK |
| Streptococcus | KYIKKNLLGVLLFDSGITAEGRRLKRTARRRYTRRRNRILYLQEIFSTEMATLDDAFFQRL |
| thermophilus | DDSFLVPDDKRDSKYPIFGNLVEEKVYHDEFPTIYHLRKYLADSTKKADLRLVYLALAHM |
| UniProtKB/ | IKYRGHFLIEGEFNSKNNDIQKNFQDFLDTYNAIFESDLSLENSKQLEEIVKDKISKLEKKD |
| Swiss-Prot: | RILKLFPGEKNSGIFSEFLKLIVGNQADFRKCFNLDEKASLHFSKESYDEDLETLLGYIGDD |
| G3ECR1.2 | YSDVFLKAKKLYDAILLSGFLTVTDNETEAPLSSAMIKRYNEHKEDLALLKEYIRNISLKT |
| Wild type | YNEVFKDDTKNGYAGYIDGKTNQEDFYVYLKNLLAEFEGADYFLEKIDREDFLRKQRTF |
| | DNGSIPYQIHLQEMRAILDKQAKFYPFLAKNKERIEKILTFRIPYYVGPLARGNSDFAWSIR |
| | KRNEKITPWNFEDVIDKESSAEAFINRMTSFDLYLPEEKVLPKHSLLYETFNVYNELTKVR |
| | FIAESMRDYQFLDSKQKKDIVRLYFKDKRKVTDKDIIEYLHAIYGYDGIELKGIEKQFNSSL |
| | STYHDLLNIINDKEFLDDSSNEAIIEEIIHTLTIFEDREMIKQRLSKFENIFDKSVLKKLSRRH |
| | YTGWGKLSAKLINGIRDEKSGNTILDYLIDDGISNRNFMQLIHDDALSFKKKIQKAQIIGDE |
| | DKGNIKEVVKSLPGSPAIKKGILQSIKIVDELVKVMGGRKPESIVVEMARENQYTNQGKSN |
| | SQQRLKRLEKSLKELGSKILKENIPAKLSKIDNNALQNDRLYLYYLQNGKDMYTGDDLDI |
| | DRLSNYDIDHIIPQAFLKDNSIDNKVLVSSASNRGKSDDFPSLEVVKKRKTFWYQLLKSKL |
| | ISQRKFDNLTKAERGGLLPEDKAGFIQRQLVETRQITKHVARLLDEKENNKKDENNRAVR |
| | TVKIITLKSTLVSQFRKDFELYKVREINDFHHAHDAYLNAVIASALLKKYPKLEPEFVYGD |
| | YPKYNSFRERKSATEKVYFYSNIMNIFKKSISLADGRVIERPLIEVNEETGESVWNKESDLA |
| | TVRRVLSYPQVNVVKKVEEQNHGLDRGKPKGLFNANLSSKPKPNSNENLVGAKEYLDPK |
| | KYGGYAGISNSFAVLVKGTIEKGAKKKITNVLEFQGISILDRINYRKDKLNFLLEKGYKDIE |
| | LIIELPKYSLFELSDGSRRMLASILSTNNKRGEIHKGNQIFLSQKFVKLLYHAKRISNTINEN |
| | HRKYVENHKKEFEELFYYILEFNENYVGAKKNGKLLNSAFQSWQNHSIDELCSSFIGPTGS |
| | ERKGLFELTSRGSAADFEFLGVKIPRYRDYTPSSLLKDATLIHQSVTGLYETRIDLAKLGEG |
| | (SEQ ID NO: 68) |
| LcCas9 | MKIKNYNLALTPSTSAVGHVEVDDDLNILEPVHHQKAIGVAKFGEGETAEARRLARSARR |
| Lactobacillus | TTKRRANRINHYFNEIMKPEIDKVDPLMFDRIKQAGLSPLDERKEFRTVIFDRPNIASYYHN |
| crispatus | QFPTIWHLQKYLMITDEKADIRLIYWALHSLLKHRGHFFNTTPMSQFKPGKLNLKDDMLA |
| NCBI Reference | LDDYNDLEGLSFAVANSPEIEKVIKDRSMHKKEKIAELKKLIVNDVPDKDLAKRNNKIITQ |
| Sequence: | IVNAIMGNSFHLNFIFDMDLDKLTSKAWSFKLDDPELDTKFDAISGSMTDNQIGIFETLQKI |
| WP_133478044.1 | YSAISLLDILNGSSNVVDAKNALYDKHKRDLNLYFKFLNTLPDEIAKTLKAGYTLYIGNRK |
| Wild type | KDLLAARKLLKVNVAKNFSQDDFYKLINKELKSIDKQGLQTRFSEKVGELVAQNNFLPV |
| | QRSSDNVFIPYQLNAITFNKILENQGKYYDFLVKPNPAKKDRKNAPYELSQLMQFTIPYYV |
| | GPLVTPEEQVKSGIPKTSRFAWMVRKDNGAITPWNFYDKVDIEATADKFIKRSIAKDSYLL |
| | SELVLPKHSLLYEKYEVFNELSNVSLDGKKLSGGVKQILFNEVFKKTNKVNTSRILKALA |
| | KHNIPGSKITGLSNPEEFTSSLQTYNAWKKYFPNQIDNFAYQQDLEKMIEWSTVFEDHKIL |
| | AKKLDEIEWLDDDQKKFVANTRLRGWGRLSKRLLTGLKDNYGKSIMQRLETTKANFQQI |
| | VYKPEFREQIDKISQAAAKNQSLEDILANSYTSPSNRKAIRKTMSVVDEYIKLNHGKEPDK |
| | IFLMFQRSEQEKGKQTEARSKQLNRILSQLKADKSANKLFSKQLADEFSNAIKKSKYKLN |
| | DKQYFYFQQLGRDALTGEVIDYDELYKYTVLHIIPRSKLTDDSQNNKVLTKYKIVDGSVA |
| | LKFGNSYSDALGMPIKAFWTELNRLKLIPKGKLLNLTTDFSTLNKYQRDGYIARQLVETQ |
| | QIVKLLATIMQSRFKHTKIIEVRNSQVANIRYQFDYFRIKNLNEYYRGFDAYLAAVVGTYL |
| | YKVYPKARRLFVYGQYLKPKKTNQENQDMHLDSEKKSQGFNFLWNLLYGKQDQIFVNG |
| | TDVIAFNRKDLITKMNTVYNYKSQKISLAIDYHNGAMFKATLFPRNDRDTAKTRKLIPKK |
| | KDYDTDIYGGYTSNVDGYMLLAEIIKRDGNKQYGFYGVPSRLVSELDTLKKTRYTEYEEK |
| | LKEIIKPELGVDLKKIKKIKILKNKVPFNQVIIDKGSKFFITSTSYRWNYRQLILSAESQQTL |
| | MDLVVDPDFSNHKARKDARKNADERLIKVYEEILYQVKNYMPMFVELHRCYEKLVDAQ |
| | KTFKSLKISDKAMVLNQILILLHSNATSPVLEKLGYHTRFTLGKKHNLISENAVLVTQSITG |
| | LKENHVSIKQML (SEQ ID NO: 69) |
| PdCas9 | MTNEKYSIGLDIGTSSIGFAVVNDNNRVIRVKGKNAIGVRLFDEGKAAADRRSFRTTRRSF |
| Pedicoccus | RTTRRRLSRRRWRLKLLREIFDAYITPVDEAFFIRLKESNLSPKDSKKQYSGDILFNDRSDK |
| damnosus | DFYEKYPTIYHLRNALMTEHRKFDVREIYLAIHHIMKFRGHFLNATPANNFKVGRLNLEE |
| NCBI Reference | KFEELNDIYQRVFPDESIEFRTDNLEQIKEVLLDNKRSRADRQRTLVSDIYQSSEDKDIEKR |
| Sequence: | NKAVATEILKASLGNKAKLNVITNVEVDKEAAKEWSITFDSESIDDDLAKIEGQMTDDGH |
| WP_062913273.1 | EIIEVLRSLYSGITLSAIVPENHTLSQSMVAKYDLHKDHLKLFKKLINGMTDTKKAKNLRA |
| Wild type | AYDGYIDGVKGKVLPQEDFYKQVQVNLDDSAEANEIQTYIDQDIFMPKQRTKANGSIPHQ |
| | LQQQELDQIIENQKAYYPWLAELNPNPDKKRQQLAKYKLDELVTFRVPYYVGPMITAKD |
| | QKNQSGAEFAWMIRKEPGNITPWNFDQKVDRMATANQFIKRMTTTDTYLLGEDVLPAQS |
| | LLYQKFEVLNELNKIRIDHKPISIEQKQQIFNDLFKQFKNVTIKHLQDYLVSQGQYSKRPLI |
| | EGLADEKRFNSSLSTYSDLCGIFGAKLVEENDRQEDLEKIIEWSTIFEDKKIYRAKLNDLT |
| | WLTDDQKEKLATKRYQGWGRLSRKLLVGLKNSEHRNIMDILWITNENFMQIQAEPDFAK |
| | LVTDANKGMLEKTDSQDVINDLYTSPQNKKAIRQILLVVHDIQNAMHGQAPAKIHVEFAR |
| | GEERNPRRSVQRQRQVEAAYEKVSNELVSAKVRQEFKEAINNKRDFKDRLFLYFMQGGI |
| | DIYTGKQLNIDQLSSYQIDHILPQAFVKDDSLTNRVLTNENQVKADSVPIDIFGKKMLSVW |
| | GRMKDQGLISKGKYRNLTMNPENISAHTENGFINRQLVETRQVIKLAVNILADEYGDSTQI |
| | ISVKADLSHQMREDFELLKNRDVNDYHHAFDAYLAAFIGNYLLKRYPKLESYFVYGDFK |
| | KFTQKETKMRRFNFIYDLKHCDQVVNKETGEILWTKDEDIKYIRHLFAYKKILVSHEVRE |
| | KRGALYNQTIYKAKDDKGSGQESKKLIRIKDDKETKIYGGYSGKSLAYMTIVQITKKNKV |
| | SYRVIGIPTLALARLNKLENDSTENNGELYKIIKPQFTHYKVDKKNGEIIETTDDFKIVVSK |
| | VRFQQLIDDAGQFFMLASDTYKNNAQQLVISNNALKAINNTNITDCPRDDLERLDNLRLD |
| | SAFDEIVKKMDKYFSAYDANNFREKIRNSNLIFYQLPVEDQWENNKITELGKRTVLTRILQ |
| | GLHANATTTDMSIFKIKTPFGQLRQRSGISLSENAQLIYQSPTGLFERRVQLNKIK |
| | (SEQ ID NO: 70) |
| FnCas9 | MKKQKFSDYYLGFDIGTNSVGWCVTDLDYNVLRFNKKDMWGSRLFEEAKTAAERRVQ |
| Fusobaterium | RNSRRRLKRRKWRLNLLEEIFSNEILKIDSNFFRRLKESSLWLEDKSSKEKFTLENDDNYK |
| nucleatum | DYDFYKQYPTIFHLRNELIKNPEKKDIRLVYLAIHSIFKSRGHFLFEGQNLKEIKNFETLYN |
| NCBI Reference | NLIAFLEDNGINKIIDKNNIEKLEKIVCDSKKGLKDKEKEFKEIFNSDKQLVAIFKLSVGSSV |
| Sequence: | SLNDLFDTDEYKKGEVEKEKISFREQIYEDDKPIYYSILGEKIELLDIAKTFYDFMVLNNILA |
| WP_060798984.1 | DSQYISEAKVKLYEEHKKDLKNLKYIIRKYNKGNYDKLFKDKNENNYSAYIGLNKEKSK |
| | KEVIEKSRLKIDDLIKNIKGYLPKVEEIEEKDKAIFNKILNKIELKTILPKQRISDNGTLPYQI |
| | HEAELEKILENQSKYYDFLNYEENGIITKDKLLMTFKFRIPYYVGPLNSYHKDKGGNSWIV |
| | RKEEGKILPWNFEQKVDIEKSAEEFIKRMTNKCTYLNGEDVIPKDTFLYSEYVILNELNKV |
| | QVNDEFLNEENKRKIIDELFKENKKVSEKKFKEYLLVKQIVDGTIELKGVKDSFNSNYISYI |
| | RFKDIFGEKLNLDIYKEISEKSILWKCLYGDDKKIFEKKIKNEYGDILTKDEIKKINTFKENN |
| | WGRLSEKLLTGIEFINLETGECYSSVMDALRRTNYNLMELLSSKFTLQESINNENKEMNEA |
| | SYRDLIEESYVSPSLKRAIFQTLKIYEEIRKITGRVPKKVFIEMARGGDESMKNKKIPARQE |
| | QLKKLYDSCGNDIANFSIDIKEMKNSLISYDNNSLRQKKLYLYYLQFGKCMYTGREIDLD |
| | RLLQNNDTYDIDHIYPRSKVIKDDSFDNLVLVLKNENAEKSNEYPVKKEIQEKMKSFWRF |
| | LKEKNFISDEKYKRLTGKDDFELRGFMARQLVNVRQTTKEVGKILQQIEPEIKIVYSKAEI |
| | ASSFREMFDFIKVRELNDTHHAKDAYLNIVAGNVYNTKFTEKPYRYLQEIKENYDVKKIY |
| | NYDIKNAWDKENSLEIVKKNMEKNTVNITRFIKEKKGQLFDLNPIKKGETSNEIISIKPKVY |
| | NGKDDKLNEKYGYYKSLNPAYFLYVEHKEKNKRIKSFERVNLVDVNNIKDEKSLVKYLI |
| | ENKKLVEPRVIKKVYKRQVILINDYPYSIVTLDSNKLMDFENLKPLFLENKYEKILKNVIKF |
| | LEDNQGKSEENYKFIYLKKKDRYEKNETLESVKDRYNLEFNEMYDKFLEKLDSKDYKNY |
| | MNNKKYQELLDVKEKFIKLNLFDKAFTLKSFLDLFNRKTMADFSKVGLTKYLGKIQKISS |
| | NVLSKNELYLLEESVTGLFVKKIKL (SEQ ID NO: 71) |
| EcCas9 | MNKYYLGLDMGSASVGWAVTDENYHLVRRKGKDLWGVRTFDVAQTAKERRITRGNRR |
| Enterococcus | RQDRRKQRIQILQELLGEEVLKTDPGFFHRMKESRYVVEDKRTLDGKQVELPYALFVDK |
| cecorum | DYTDKEYYKQFPTINHLIVYLMTTSDTPDIRLVYLALHYYMKNRGNFLHSGDINNVKDIN |
| NCBI Reference | DILEQLDNVLETFLDGWNLKLKSYVEDIKNIYNRDLGRGERKKAFVNTLGAKTKAEKAF |
| Sequence: | CSLISGGSTNLAELFDDSSLKEIETPKIEFASSSLEDKIDGIQEALEDRFAVIEAAKRLYDWK |
| WP_047338501.1 | TLTDILGDSSSLAEARVNSYQMHHEQLLELKSLVKEYLDRKVFQEVFVSLNVANNYPAYI |
| Wild type | GHTKINGKKKELEVKRTKRNDFYSYVKKQVIEPIKKKVSDEAVLTKLSEIESLIEVDKYLP |
| | LQVNSDNGVIPYQVKLNELTRIFDNLENRIPVLRENRDKIIKTFKFRIPYYVGSLNGVVKNG |
| | KCTNWMVRKEEGKIYPWNFEDKVDLEASAEQFIRRMTNKCTYLVNEDVLPKYSLLYSKY |
| | LVLSELNNLRIDGRPLDVKIKQDIYENVFKKNRKVTLKKIKKYLLKEGIITDDDALSGLAD |
| | DVKSSLTAYRDFKEKLGHLDLSEAQMENIILNITLFGDDKKLLKKRLAALYPFIDDKSLNR |
| | IATLNYRDWGRLSERFLSGITSVDQETGELRTIIQCMYETQANLMQLLAEPYHFVEAIEKE |
| | NPKVDLESISYRIVNDLYVSPAVKRQIWQTLLVIKDIKQVMKHDPERIFIEMAREKQESKK |
| | TKSRKQVLSEVYKKAKEYEHLFEKLNSLTEEQLRSKKIYLYFTQLGKCMYSGEPIDFENL |
| | VSANSNYDIDHIYPQSKTIDDSFNNIVLVKKSLNAYKSNHYPIDKNIRDNEKVKTLWNTLV |
| | SKGLITKEKYERLIRSTPFSDEELAGFIARQLVETRQSTKAVAEILSNWFPESEIVYSKAKN |
| | VSNFRQDFEILKVRELNDCHHAHDAYLNIVVGNAYHTKFTNSPYRFIKNKANQEYNLRKL |
| | LQKVNKIESNGVVAWVGQSENNPGTIATVKKVIRRNTVLISRMVKEVDGQLFDLTLMKK |
| | GKGQVPIKSSDERLTDISKYGGYNKATGAYFTFVKSKKRGKVVRSFEYVPLHLSKQFENN |
| | NELLKEYIEKDRGLTDVEILIPKVLINSLFRYNGSLVRITGRGDTRLLLVHEQPLYVSNSFV |
| | QQLKSVSSYKLKKSENDNAKLTKTATEKLSNIDELYDGLLRKLDLPIYSYWFSSIKEYLVE |
| | SRTKYIKLSIEEKALVIFEILHLFQSDAQVPNLKILGLSTKPSRIRIQKNLKDTDKMSIIHQSP |
| | SGIFEHEIELTSL (SEQ ID NO: 72) |
| AhCas9 | MQNGFLGITVSSEQVGWAVTNPKYELERASRKDLWGVRLFDKAETAEDRRMFRTNRRL |
| Anaerostipes | NQRKKNRIHYLRDIFHEEVNQKDPNFFQQLDESNFCEDDRTVEFNFDTNLYKNQFPTVYH |
| hadrus | LRKYLMETKDKPDIRLVYLAFSKFMKNRGHFLYKGNLGEVMDFENSMKGFCESLEKFNI |
| NCBI Reference | DFPTLSDEQVKEVRDILCDHKIAKTVKKKNIITITKVKSKTAKAWIGLFCGCSVPVKVLFQ |
| Sequence: | DIDEEIVTDPEKISFEDASYDDYIANIEKGVGIYYEAIVSAKMLFDWSILNEILGDHQLLSDA |
| WP_044924278.1 | MIAEYNKHHDDLKRLQKIIKGTGSRELYQDIFINDVSGNYVCYVGHAKTMSSADQKQFY |
| Wild type | TFLKNRLKNVNGISSEDAEWIDTEIKNGTLLPKQTKRDNSVIPHQLQLREFELILDNMQEM |
| | YPFLKENREKLLKIFNFVIPYYVGPLKGVVRKGESTNWMVPKKDGVIHPWNFDEMVDKE |
| | ASAECFISRMTGNCSYLFNEKVLPKNSLLYETFEVLNELNPLKINGEPISVELKQRIYEQLF |
| | LTGKKVTKKSLTKYLIKNGYDKDIELSGIDNEFHSNLKSHIDFEDYDNLSDEEVEQIILRITV |
| | FEDKQLLKDYLNREFVKLSEDERKQICSLSYKGWGNLSEMLLNGITVTDSNGVEVSVMD |
| | MLWNTNLNLMQILSKKYGYKAEIEHYNKEHEKTIYNREDLMDYLNIPPAQRRKVNQLITI |
| | VKSLKKTYGVPNKIFFKISREHQDDPKRTSSRKEQLKYLYKSLKSEDEKHLMKELDELND |
| | HELSNDKVYLYFLQKGRCIYSGKKLNLSRLRKSNYQNDIDYIYPLSAVNDRSMNNKVLTG |
| | IQENRADKYTYFPVDSEIQKKMKGFWMELVLQGFMTKEKYFRLSRENDFSKSELVSFIER |
| | EISDNQQSGRMIASVLQYYFPESKIVFVKEKLISSFKRDFHLISSYGHNHLQAAKDAYITIV |
| | VGNVYHTKFTMDPAIYFKNHKRKDYDLNRLFLENISRDGQIAWESGPYGSIQTVRKEYAQ |
| | NHIAVTKRVVEVKGGLFKQMPLKKGHGEYPLKTNDPRFGNIAQYGGYTNVTGSYFVLVE |
| | SMEKGKKRISLEYVPVYLHERLEDDPGHKLLKEYLVDHRKLNHPKILLAKVRKNSLLKID |
| | GFYYRLNGRSGNALILTNAVELIMDDWQTKTANKISGYMKRRAIDKKARVYQNEFHIQE |
| | LEQLYDFYLDKLKNGVYKNRKNNQAELIHNEKEQFMELKTEDQCVLLTEIKKLFVCSPM |
| | QADLTLIGGSKHTGMIAMSSNVTKADFAVIAEDPLGLRNKVIYSHKGEK (SEQ ID NO: 73) |
| KvCas9 | MSQNNNKIYNIGLDIGDASVGWAVVDEHYNLLKRHGKHMWGSRLFTQANTAVERRSSR |
| Kandleria | STRRRYNKRRERIRLLREIMEDMVLDVDPTFFIRLANVSFLDQEDKKDYLKENYHSNYNL |
| vitulina | FIDKDFNDKTYYDKYPTIYHLRKHLCESKEKEDPRLIYLALHHIVKYRGNFLYEGQKFSM |
| NCBI Reference | DVSNIEDKMIDVLRQFNEINLFEYVEDRKKIDEVLNVLKEPLSKKHKAEKAFALFDTTKD |
| Sequence: | NKAAYKELCAALAGNKFNVTKMLKEAELHDEDEKDISFKFSDATFDDAFVEKQPLLGDC |
| WP_031589969.1 | VEFIDLLHDIYSWVELQNILGSAHTSEPSISAAMIQRYEDHKNDLKLLKDVIRKYLPKKYF |
| Wild type | EVFRDEKSKKNNYCNYINHPSKTPVDEFYKYIKKLIEKIDDPDVKTILNKIELESFMLKQNS |
| | RTNGAVPYQMQLDELNKILENQSVYYSDLKDNEDKIRSILTFRIPYYFGPLNITKDRQFDW |
| | IIKKEGKENERILPWNANEIVDVDKTADEFIKRMRNFCTYFPDEPVMAKNSLTVSKYEVL |
| | NEINKLRINDHLIKRDMKDKMLHTLFMDHKSISANAMKKWLVKNQYFSNTDDIKIEGFQ |
| | KENACSTSLTPWIDFTKIFGKINESNYDFIEKIIYDVTVFEDKKILRRRLKKEYDLDEEKIKK |
| | ILKLKYSGWSRLSKKLLSGIKTKYKDSTRTPETVLEVMERTNMNLMQVINDEKLGFKKTI |
| | DDANSTSVSGKFSYAEVQELAGSPAIKRGIWQALLIVDEIKKIMKHEPAHVYIEFARNEDE |
| | KERKDSFVNQMLKLYKDYDFEDETEKEANKHLKGEDAKSKIRSERLKLYYTQMGKCMY |
| | TGKSLDIDRLDTYQVDHIVPQSLLKDDSIDNKVLVLSSENQRKLDDLVIPSSIRNKMYGFW |
| | EKLFNNKIISPKKFYSLIKTEFNEKDQERFINRQIVETRQITKHVAQIIDNHYENTKVVTVRA |
| | DLSHQFRERYHIYKNRDINDFHHAHDAYIATILGTYIGHRFESLDAKYIYGEYKRIFRNQK |
| | NKGKEMKKNNDGFILNSMRNIYADKDTGEIVWDPNYIDRIKKCFYYKDCFVTKKLEENN |
| | GTFFNVTVLPNDTNSDKDNTLATVPVNKYRSNVNKYGGFSGVNSFIVAIKGKKKKGKKV |
| | IEVNKLTGIPLMYKNADEEIKINYLKQAEDLEEVQIGKEILKNQLIEKDGGLYYIVAPTEIIN |
| | AKQLILNESQTKLVCEIYKAMKYKNYDNLDSEKIIDLYRLLINKMELYYPEYRKQLVKKF |
| | EDRYEQLKVISIEEKCNIIKQILATLHCNSSIGKIMYSDFKISTTIGRLNGRTISLDDISFIAESP |
| | TGMYSKKYKL (SEQ ID NO: 74) |
| EfCas9 | MRLFEEGHTAEDRRLKRTARRRISRRRNRLRYLQAFFEEAMTDLDENFFARLQESFLVPE |
| Enterococcus | DKKWHRHPIFAKLEDEVAYHETYPTIYHLRKKLADSSEQADLRLIYLALAHIVKYRGHFLI |
| faecalis | EGKLSTENTSVKDQFQQFMVIYNQTFVNGESRLVSAPLPESVLIEEELTEKASRTKKSEKV |
| NCBI Reference | LQQFPQEKANGLFGQFLKLMVGNKADFKKVFGLEEEAKITYASESYEEDLEGILAKVGDE |
| Sequence: | YSDVFLAAKNVYDAVELSTILADSDKKSHAKLSSSMIVRFTEHQEDLKKFKRFIRENCPDE |
| WP_016631044.1 | YDNLFKNEQKDGYAGYIAHAGKVSQLKFYQYVKKIIQDIAGAEYFLEKIAQENFLRKQRT |
| Wild type | FDNGVIPHQIHLAELQAIIHRQAAYYPFLKENQEKIEQLVTFRIPYYVGPLSKGDASTFAWL |
| | KRQSEEPIRPWNLQETVDLDQSATAFIERMTNFDTYLPSEKVLPKHSLLYEKFMVFNELTK |
| | ISYTDDRGIKANFSGKEKEKIFDYLFKTRRKVKKKDIIQFYRNEYNTEIVTLSGLEEDQFNA |
| | SFSTYQDLLKCGLTRAELDHPDNAEKLEDIIKILTIFEDRQRIRTQLSTFKGQFSAEVLKKLE |
| | RKHYTGWGRLSKKLINGIYDKESGKTILDYLVKDDGVSKHYNRNFMQLINDSQLSFKNAI |
| | QKAQSSEHEETLSETVNELAGSPAIKKGIYQSLKIVDELVAIMGYAPKRIVVEMARENQTT |
| | STGKRRSIQRLKIVEKAMAEIGSNLLKEQPTTNEQLRDTRLFLYYMQNGKDMYTGDELSL |
| | HRLSHYDIDHIIPQSFMKDDSLDNLVLVGSTENRGKSDDVPSKEVVKDMKAYWEKLYAA |
| | GLISQRKFQRLTKGEQGGLTLEDKAHFIQRQLVETRQITKNVAGILDQRYNAKSKEKKVQI |
| | ITLKASLTSQFRSIFGLYKVREVNDYHHGQDAYLNCVVATTLLKVYPNLAPEFVYGEYPK |
| | FQTFKENKATAKAIIYTNLLRFFTEDEPRFTKDGEILWSNSYLKTIKKELNYHQMNIVKKV |
| | EVQKGGFSKESIKPKGPSNKLIPVKNGLDPQKYGGFDSPVVAYTVLFTHEKGKKPLIKQEI |
| | LGITIMEKTRFEQNPILFLEEKGFLRPRVLMKLPKYTLYEFPEGRRRLLASAKEAQKGNQM |
| | VLPEHLLTLLYHAKQCLLPNQSESLAYVEQHQPEFQEILERVVDFAEVHTLAKSKVQQIV |
| | KLFEANQTADVKEIAASFIQLMQFNAMGAPSTFKFFQKDIERARYTSIKEIFDATIIYQSPTG |
| | LYETRRKVVD (SEQ ID NO: 75) |
| Staphylococcus | KRNYILGLDIGITSVGYGIIDYETRDVIDAGVRLFKEANVENNEGRRSKRGARRLKRRRRH |
| aureus Cas9 | RIQRVKKLLFDYNLLTDHSELSGINPYEARVKGLSQKLSEEEFSAALLHLAKRRGVHNVN |
| | EVEEDTGNELSTKEQISRNSKALEEKYVAELQLERLKKDGEVRGSINRFKTSDYVKEAKQ |
| | LLKVQKAYHQLDQSFIDTYIDLLETRRTYYEGPGEGSPFGWKDIKEWYEMLMGHCTYFP |
| | EELRSVKYAYNADLYNALNDLNNLVITRDENEKLEYYEKFQIIENVFKQKKKPTLKQIAK |
| | EILVNEEDIKGYRVTSTGKPEFTNLKVYHDIKDITARKEIIENAELLDQIAKILTIYQSSEDIQ |
| | EELTNLNSELTQEEIEQISNLKGYTGTHNLSLKAINLILDELWHTNDNQIAIFNRLKLVPKK |
| | VDLSQQKEIPTTLVDDFILSPVVKRSFIQSIKVINAIIKKYGLPNDIIIELAREKNSKDAQKMI |
| | NEMQKRNRQTNERIEEIIRTTGKENAKYLIEKIKLHDMQEGKCLYSLEAIPLEDLLNNPEN |
| | YEVDHIIPRSVSFDNSFNNKVLVKQEENSKKGNRTPFQYLSSSDSKISYETFKKHILNLAKG |
| | KGRISKTKKEYLLEERDINRFSVQKDFINRNLVDTRYATRGLMNLLRSYFRVNNLDVKVK |
| | SINGGFTSFLRRKWKFKKERNKGYKHHAEDALIIANADFIFKEWKKLDKAKKVMENQMF |
| | EEKQAESMPEIETEQEYKEIFITPHQIKHIKDFKDYKYSHRVDKKPNRELINDTLYSTRKDD |
| | KGNTLIVNNLNGLYDKDNDKLKKLINKSPEKLLMYHHDPQTYQKLKLIMEQYGDEKNPL |
| | YKYYEETGNYLTKYSKKDNGPVIKKIKYYGNKLNAHLDITDDYPNSRNKVVKLSLKPYR |
| | FDVYLDNGVYKFVTVKNLDVIKKENYYEVNSKCYEEAKKLKKISNQAEFIASFYNNDLIK |
| | INGELYRVIGVNNDLLNRIEVNMIDITYREYLENMNDKRPPRIIKTIASKTQSIKKYSTDILG |
| | NLYEVKSKKHPQIIKKG (SEQ ID NO: 76) |
| Geobacillus | MKYKIGLDIGITSIGWAVINLDIPRIEDLGVRIFDRAENPKTGESLALPRRLARSARRRLRRR |
| thermodenitrificans | KHRLERIRRLFVREGILTKEELNKLFEKKHEIDVWQLRVEALDRKLNNDELARILLHLAKR |
| Cas9 | RGFRSNRKSERTNKENSTMLKHIEENQSILSSYRTVAEMVVKDPKFSLHKRNKEDNYTNT |
| | VARDDLEREIKLIFAKQREYGNIVCTEAFEHEYISIWASQRPFASKDDIEKKVGFCTFEPKE |
| | KRAPKATYTFQSFTVWEHINKLRLVSPGGIRALTDDERRLIYKQAFHKNKITFHDVRTLLN |
| | LPDDTRFKGLLYDRNTTLKENEKVRFLELGAYHKIRKAIDSVYGKGAAKSFRPIDFDTFG |
| | YALTMFKDDTDIRSYLRNEYEQNGKRMENLADKVYDEELIEELLNLSFSKFGHLSLKALR |
| | NILPYMEQGEVYSTACERAGYTFTGPKKKQKTVLLPNIPPIANPVVMRALTQARKVVNAII |
| | KKYGSPVSIHIELARELSQSFDERRKMQKEQEGNRKKNETAIRQLVEYGLTLNPTGLDIVK |
| | FKLWSEQNGKCAYSLQPIEIERLLEPGYTEVDHVIPYSRSLDDSYTNKVLVLTKENREKGN |
| | RTPAEYLGLGSERWQQFETFVLTNKQFSKKKRDRLLRLHYDENEENEFKNRNLNDTRYIS |
| | RFLANFIREHLKFADSDDKQKVYTVNGRITAHLRSRWNFNKNREESNLHHAVDAAIVAC |
| | TTPSDIARVTAFYQRREQNKELSKKTDPQFPQPWPHFADELQARLSKNPKESIKALNLGN |
| | YDNEKLESLQPVFVSRMPKRSITGAAHQETLRRYIGIDERSGKIQTVVKKKLSEIQLDKTG |
| | HFPMYGKESDPRTYEAIRQRLLEHNNDPKKAFQEPLYKPKKNGELGPIIRTIKIIDTTNQVIP |
| | LNDGKTVAYNSNIVRVDVFEKDGKYYCVPIYTIDMMKGILPNKAIEPNKPYSEWKEMTE |
| | DYTFRFSLYPNDLIRIEFPREKTIKTAVGEEIKIKDLFAYYQTIDSSNGGLSLVSHDNNFSLR |
| | SIGSRTLKRFEKYQVDVLGNIYKVRGEKRVGVASSSHSKAGETIRPL (SEQ ID NO: 77) |
| ScCas9 | MEKKYSIGLDIGTNSVGWAVITDDYKVPSKKFKVLGNTNRKSIKKNLMGALLFDSGETAE |
| S. canis | ATRLKRTARRRYTRRKNRIRYLQEIFANEMAKLDDSFFQRLEESFLVEEDKKNERHPIFGN |
| 1375 AA | LADEVAYHRNYPTIYHLRKKLADSPEKADLRLIYLALAHIIKFRGHFLIEGKLNAENSDVA |
| 159.2 kDa | KLFYQLIQTYNQLFEESPLDEIEVDAKGILSARLSKSKRLEKLIAVFPNEKKNGLFGNIIALA |
| | LGLTPNFKSNFDLTEDAKLQLSKDTYDDDLDELLGQIGDQYADLFSAAKNLSDAILLSDIL |
| | RSNSEVTKAPLSASMVKRYDEHHQDLALLKTLVRQQFPEKYAEIFKDDTKNGYAGYVGI |
| | GIKHRKRTTKLATQEEFYKFIKPILEKMDGAEELLAKLNRDDLLRKQRTFDNGSIPHQIHL |
| | KELHAILRRQEEFYPFLKENREKIEKILTFRIPYYVGPLARGNSRFAWLTRKSEEAITPWNF |
| | EEVVDKGASAQSFIERMTNFDEQLPNKKVLPKHSLLYEYFTVYNELTKVKYVTERMRKP |
| | EFLSGEQKKAIVDLLFKTNRKVTVKQLKEDYFKKIECFDSVEIIGVEDRFNASLGTYHDLL |
| | KIIKDKDFLDNEENEDILEDIVLTLTLFEDREMIEERLKTYAHLFDDKVMKQLKRRHYTG |
| | WGRLSRKMINGIRDKQSGKTILDFLKSDGFSNRNFMQLIHDDSLTFKEEIEKAQVSGQGDS |
| | LHEQIADLAGSPAIKKGILQTVKIVDELVKVMGHKPENIVIEMARENQTTTKGLQQSRERK |
| | KRIEEGIKELESQILKENPVENTQLQNEKLYLYYLQNGRDMYVDQELDINRLSDYDVDHI |
| | VPQSFIKDDSIDNKVLTRSVENRGKSDNVPSEEVVKKMKNYWRQLLNAKLITQRKFDNLT |
| | KAERGGLSEADKAGFIKRQLVETRQITKHVARILDSRMNTKRDKNDKPIREVKVITLKSKL |
| | VSDFRKDFQLYKVRDINNYHHAHDAYLNAVVGTALIKKYPKLESEFVYGDYKVYDVRK |
| | MIAKSEQEIGKATAKRFFYSNIMNFFKTEVKLANGEIRKRPLIETNGETGEVVWNKEKDFA |
| | TVRKVLAMPQVNIVKKTEVQTGGFSKESILSKRESAKLIPRKKGWDTRKYGGFGSPTVAY |
| | SILVVAKVEKGKAKKLKSVKVLVGITIMEKGSYEKDPIGFLEAKGYKDIKKELIFKLPKYS |
| | LFELENGRRRMLASATELQKANELVLPQHLVRLLYYTQNISATTGSNNLGYIEQHREEFK |
| | EIFEKIIDFSEKYILKNKVNSNLKSSFDEQFAVSDSILLSNSFVSLLKYTSFGASGGFTFLDLD |
| | VKQGRLRYQTVTEVLDATLIYQSITGLYETRTDLSQLGGD (SEQ ID NO: 78) |

The base editors described herein may include any of the above Cas9 ortholog sequences, or any variants thereof having at least 80%, at least 85%, at least 90%, at least 95%, or at least 99% sequence identity thereto.

The napDNAbp may include any suitable homologs and/or orthologs or naturally occurring enzymes, such as, Cas9. Cas9 homologs and/or orthologs have been described in various species, including, but not limited to, S. pyogenes and S. thermophilus. Preferably, the Cas moiety is configured (e.g, mutagenized, recombinantly engineered, or otherwise obtained from nature) as a nickase, i.e., capable of cleaving only a single strand of the target double strand DNA. Suitable Cas9 nucleases (including nickase variants and nuclease inactive variants) and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease has an inactive (e.g., an inactivated) DNA cleavage domain, that is, the Cas9 is a nickase. In some embodiments, the Cas9 protein comprises an amino acid sequence that is at least 80% identical to the amino acid sequence of a Cas9 protein as provided by any one of the variants of Table 3. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the Cas9 orthologs in the above tables.

### (3) Dead Cas9 Variant

In certain embodiments, the base editors described herein may include a dead Cas9, e.g., dead SpCas9, which has no nuclease activity due to one or more mutations that inactive both nuclease domains of Cas9, namely the RuvC domain (which cleaves the non-protospacer DNA strand) and HNH domain (which cleaves the protospacer DNA strand). The nuclease inactivation may be due to one or mutations that result in one or more substitutions and/or deletions in the amino acid sequence of the encoded protein, or any variants thereof having at least 80%, at least 85%, at least 90%, at least 95%, or at least 99% sequence identity thereto.

As used herein, the term “dCas9” refers to a nuclease-inactive Cas9 or nuclease-dead Cas9, or a functional fragment thereof, and embraces any naturally occurring dCas9 from any organism, any naturally-occurring dCas9 equivalent or functional fragment thereof, any dCas9 homolog, ortholog, or paralog from any organism, and any mutant or variant of a dCas9, naturally-occurring or engineered. The term dCas9 is not meant to be particularly limiting and may be referred to as a “dCas9 or equivalent.” Exemplary dCas9 proteins and method for making dCas9 proteins are further described herein and/or are described in the art and are incorporated herein by reference.

In other embodiments, dCas9 corresponds to, or comprises in part or in whole, a Cas9 amino acid sequence having one or more mutations that inactivate the Cas9 nuclease activity. In other embodiments, Cas9 variants having mutations other than D10A and H840A are provided which may result in the full or partial inactivate of the endogenous Cas9 nuclease activity (e.g., nCas9 or dCas9, respectively). Such mutations, by way of example, include other amino acid substitutions at D10 and H820, or other substitutions within the nuclease domains of Cas9 (e.g., substitutions in the HNH nuclease subdomain and/or the RuvC1 subdomain) with reference to a wild type sequence such as Cas9 from Streptococcus pyogenes (NCBI Reference Sequence: NC_017053.1). In some embodiments, variants or homologues of Cas9 (e.g., variants of Cas9 from Streptococcus pyogenes (NCBI Reference Sequence: NC_017053.1)) are provided which are at least about 70% identical, at least about 80% identical, at least about 90% identical, at least about 95% identical, at least about 98% identical, at least about 99% identical, at least about 99.5% identical, or at least about 99.9% identical to NCBI Reference Sequence: NC_017053.1. In some embodiments, variants of dCas9 (e.g., variants of NCBI Reference Sequence: NC_017053.1) are provided having amino acid sequences which are shorter, or longer than NC_017053.1 by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more.

In one embodiment, the dead Cas9 may be based on the canonical SpCas9 sequence of Q99ZW2 and may have the following sequence, which comprises a D10A and an H810A substitutions (underlined and bolded), or a variant be variant of SEQ ID NO: 79 having at least 80%, at least 85%, at least 90%, at least 95%, or at least 99% sequence identity thereto:

| | | SEQ |
|---|---|---|
| | | ID |
| Description | Sequence | NO: |
| dead Cas9 or | MDKKYSIGLXIGTNSVGWAVITDEYKVPSKKFKVLGNTDRHSIKKNLIGALL | 79 |
| dCas9 | FDSGETAEATRLKRTARRRYTRRKNRICYLQEIFSNEMAKVDDSFFHRLEESF | |
| Streptococcus | LVEEDKKHERHPIFGNIVDEVAYHEKYPTIYHLRKKLVDSTDKADLRLIYLAL | |
| pyogenes | AHMIKFRGHFLIEGDLNPDNSDVDKLFIQLVQTYNQLFEENPINASGVDAKAI | |
| Q99ZW2 Cas9 | LSARLSKSRRLENLIAQLPGEKKNGLFGNLIALSLGLTPNFKSNFDLAEDAKL | |
| with D10X | QLSKDTYDDDLDNLLAQIGDQYADLFLAAKNLSDAILLSDILRVNTEITKAPL | |
| and | SASMIKRYDEHHQDLTLLKALVRQQLPEKYKEIFFDQSKNGYAGYIDGGASQE | |
| H810X | EFYKFIKPILEKMDGTEELLVKLNREDLLRKQRTFDNGSIPHQIHLGELHAIL | |
| Where | RRQEDFYPFLKDNREKIEKILTFRIPYYVGPLARGNSRFAWMTRKSEETITPW | |
| “X” is | NFEEVVDKGASAQSFIERMTNFDKNLPNEKVLPKHSLLYEYFTVYNELTKVKY | |
| any amino | VTEGMRKPAFLSGEQKKAIVDLLFKTNRKVTVKQLKEDYFKKIECFDSVEISG | |
| acid | VEDRFNASLGTYHDLLKIIKDKDFLDNEENEDILEDIVLTLTLFEDREMIEER | |
| | LKTYAHLFDDKVMKQLKRRRYTGWGRLSRKLINGIRDKQSGKTILDFLKSDGF | |
| | ANRNFMQLIHDDSLTFKEDIQKAQVSGQGDSLHEHIANLAGSPAIKKGILQTV | |
| | KVVDELVKVMGRHKPENIVIEMARENQTTQKGQKNSRERMKRIEEGIKELGS | |
| | QILKEHPVENTQLQNEKLYLYYLQNGRDMYVDQELDINRLSDYDVDX | |
| | IVPQSFLKDDSIDNKVLTRSDKNRGKSDNVPSEEVVKKMKNYWRQLLNAKL | |
| | ITQRKFDNLTKAERGGLSELDKAGFIKRQLVETRQITKHVAQILDSRMNTKY | |
| | DENDKLIREVKVITLKSKLVSDFRKDFQFYKVREINNYHHAHDAYLNAVVG | |
| | TALIKKYPKLESEFVYGDYKVYDVRKMIAKSEQEIGKATAKYFFYSNIMNFF | |
| | KTEITLANGEIRKRPLIETNGETGEIVWDKGRDFATVRKVLSMPQVNIVKKTE | |
| | VQTGGFSKESILPKRNSDKLIARKKDWDPKKYGGFDSPTVAYSVLVVAKVEK | |
| | GKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQKQ | |
| | LFVEQHKHYLDEIIEQISEFSKRVILADANLDKVLSAYNKHRDKPIREQAENI | |
| | IHLFTLTNLGAPAAFKYFDTTIDRKRYTSTKEVLDATLIHQSITGLYETRIDL | |
| | SQLGGD | |
| dead Cas9 or | MDKKYSIGLAIGTNSVGWAVITDEYKVPSKKFKVLGNTDRHSIKKNLIGALL | 80 |
| dCas9 | FDSGETAEATRLKRTARRRYTRRKNRICYLQEIFSNEMAKVDDSFFHRLEESF | |
| Streptococcus | LVEEDKKHERHPIFGNIVDEVAYHEKYPTIYHLRKKLVDSTDKADLRLIYLAL | |
| pyogenes | AHMIKFRGHFLIEGDLNPDNSDVDKLFIQLVQTYNQLFEENPINASGVDAKAI | |
| Q99ZW2 Cas9 | LSARLSKSRRLENLIAQLPGEKKNGLFGNLIALSLGLTPNFKSNFDLAEDAKL | |
| with D10A | QLSKDTYDDDLDNLLAQIGDQYADLFLAAKNLSDAILLSDILRVNTEITKAPL | |
| and | SASMIKRYDEHHQDLTLLKALVRQQLPEKYKEIFFDQSKNGYAGYIDGGASQE | |
| H810A | EFYKFIKPILEKMDGTEELLVKLNREDLLRKQRTFDNGSIPHQIHLGELHAIL | |
| | RRQEDFYPFLKDNREKIEKILTFRIPYYVGPLARGNSRFAWMTRKSEETITPW | |
| | NFEEVVDKGASAQSFIERMTNFDKNLPNEKVLPKHSLLYEYFTVYNELTKVKY | |
| | VTEGMRKPAFLSGEQKKAIVDLLFKTNRKVTVKQLKEDYFKKIECFDSVEISG | |
| | VEDRFNASLGTYHDLLKIIKDKDFLDNEENEDILEDIVLTLTLFEDREMIEER | |
| | LKTYAHLFDDKVMKQLKRRRYTGWGRLSRKLINGIRDKQSGKTILDFLKSDGF | |
| | ANRNFMQLIHDDSLTFKEDIQKAQVSGQGDSLHEHIANLAGSPAIKKGIL | |
| | QTVKVVDELVKVMGRHKPENIVIEMARENQTTQKGQKNSRERMKRIEEGIK | |
| | ELGSQILKEHPVENTQLQNEKLYLYYLQNGRDMYVDQELDINRLSDYDVDA | |
| | IVPQSFLKDDSIDNKVLTRSDKNRGKSDNVPSEEVVKKMKNYWRQLLNAKL | |
| | ITQRKFDNLTKAERGGLSELDKAGFIKRQLVETRQITKHVAQILDSRMNTKY | |
| | DENDKLIREVKVITLKSKLVSDFRKDFQFYKVREINNYHHAHDAYLNAVVG | |
| | TALIKKYPKLESEFVYGDYKVYDVRKMIAKSEQEIGKATAKYFFYSNIMNFF | |
| | KTEITLANGEIRKRPLIETNGETGEIVWDKGRDFATVRKVLSMPQVNIVKKTE | |
| | VQTGGFSKESILPKRNSDKLIARKKDWDPKKYGGFDSPTVAYSVLVVAKVEK | |
| | GKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQKQ | |
| | LFVEQHKHYLDEIIEQISEFSKRVILADANLDKVLSAYNKHRDKPIREQAENI | |
| | IHLFTLTNLGAPAAFKYFDTTIDRKRYTSTKEVLDATLIHQSITGLYETRID | |
| | LSQLGGD | |

### (4) Cas9 Nickase Variant

In one embodiment, the base editors described herein comprise a Cas9 nickase. The term “Cas9 nickase” of “nCas9” refers to a variant of Cas9 which is capable of introducing a single-strand break in a double strand DNA molecule target. In some embodiments, the Cas9 nickase comprises only a single functioning nuclease domain. The wild type Cas9 (e.g., the canonical SpCas9) comprises two separate nuclease domains, namely, the RuvC domain (which cleaves the non-protospacer DNA strand) and HNH domain (which cleaves the protospacer DNA strand). In one embodiment, the Cas9 nickase comprises a mutation in the RuvC domain which inactivates the RuvC nuclease activity. For example, mutations in aspartate (D) 10, histidine (H) 983, aspartate (D) 986, or glutamate (E) 762, have been reported as loss-of-function mutations of the RuvC nuclease domain and the creation of a functional Cas9 nickase (e.g., Nishimasu et al., “Crystal structure of Cas9 in complex with guide RNA and target DNA,” Cell 156(5), 935-949, which is incorporated herein by reference). Thus, nickase mutations in the RuvC domain could include D10X, H983X, D986X, or E762X, wherein X is any amino acid other than the wild type amino acid. In certain embodiments, the nickase could be D10A, of H983A, or D986A, or E762A, or a combination thereof.

In various embodiments, the Cas9 nickase can having a mutation in the RuvC nuclease domain and have one of the following amino acid sequences, or a variant thereof having an amino acid sequence that has at least 80%, at least 85%, at least 90%, at least 95%, or at least 99% sequence identity thereto.

| | | SEQ |
|---|---|---|
| | | ID |
| Description | Sequence | NO: |
| Cas9 nickase | MDKKYSIGLXIGTNSVGWAVITDEYKVPSKKFKVLGNTDRHSIKKNLIGALL | 81 |
| Streptococcus | FDSGETAEATRLKRTARRRYTRRKNRICYLQEIFSNEMAKVDDSFFHRLEESF | |
| pyogenes | LVEEDKKHERHPIFGNIVDEVAYHEKYPTIYHLRKKLVDSTDKADLRLIYLAL | |
| Q99ZW2 Cas9 | AHMIKFRGHFLIEGDLNPDNSDVDKLFIQLVQTYNQLFEENPINASGVDAKAI | |
| with D10X, | LSARLSKSRRLENLIAQLPGEKKNGLFGNLIALSLGLTPNFKSNFDLAEDAKL | |
| wherein X is | QLSKDTYDDDLDNLLAQIGDQYADLFLAAKNLSDAILLSDILRVNTEITKAPL | |
| any alternate | SASMIKRYDEHHQDLTLLKALVRQQLPEKYKEIFFDQSKNGYAGYIDGGASQ | |
| amino acid | EEFYKFIKPILEKMDGTEELLVKLNREDLLRKQRTFDNGSIPHQIHLGELHAIL | |
| | RRQEDFYPFLKDNREKIEKILTFRIPYYVGPLARGNSRFAWMTRKSEETITPW | |
| | NFEEVVDKGASAQSFIERMTNFDKNLPNEKVLPKHSLLYEYFTVYNELTKVK | |
| | YVTEGMRKPAFLSGEQKKAIVDLLFKTNRKVTVKQLKEDYFKKIECFDSVEI | |
| | SGVEDRFNASLGTYHDLLKIIKDKDFLDNEENEDILEDIVLTLTLFEDREMIEE | |
| | RLKTYAHLFDDKVMKQLKRRRYTGWGRLSRKLINGIRDKQSGKTILDFLKS | |
| | DGFANRNFMQLIHDDSLTFKEDIQKAQVSGQGDSLHEHIANLAGSPAIKKGIL | |
| | QTVKVVDELVKVMGRHKPENIVIEMARENQTTQKGQKNSRERMKRIEEGIK | |
| | ELGSQILKEHPVENTQLQNEKLYLYYLQNGRDMYVDQELDINRLSDYDVDH | |
| | IVPQSFLKDDSIDNKVLTRSDKNRGKSDNVPSEEVVKKMKNYWRQLLNAKL | |
| | ITQRKFDNLTKAERGGLSELDKAGFIKRQLVETRQITKHVAQILDSRMNTKY | |
| | DENDKLIREVKVITLKSKLVSDFRKDFQFYKVREINNYHHAHDAYLNAVVG | |
| | TALIKKYPKLESEFVYGDYKVYDVRKMIAKSEQEIGKATAKYFFYSNIMNFF | |
| | KTEITLANGEIRKRPLIETNGETGEIVWDKGRDFATVRKVLSMPQVNIVKKTE | |
| | VQTGGFSKESILPKRNSDKLIARKKDWDPKKYGGFDSPTVAYSVLVVAKVE | |
| | KGKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQK | |
| | QLFVEQHKHYLDEIIEQISEFSKRVILADANLDKVLSAYNKHRDKPIREQAENI | |
| | IHLFTLTNLGAPAAFKYFDTTIDRKRYTSTKEVLDATLIHQSITGLYETRIDLS | |
| | QLGGD | |
| Cas9 nickase | MDKKYSIGLDIGTNSVGWAVITDEYKVPSKKFKVLGNTDRHSIKKNLIGALL | 82 |
| Streptococcus | FDSGETAEATRLKRTARRRYTRRKNRICYLQEIFSNEMAKVDDSFFHRLEESF | |
| pyogenes | LVEEDKKHERHPIFGNIVDEVAYHEKYPTIYHLRKKLVDSTDKADLRLIYLAL | |
| Q99ZW2 Cas9 | AHMIKFRGHFLIEGDLNPDNSDVDKLFIQLVQTYNQLFEENPINASGVDAKAI | |
| with E762X, | LSARLSKSRRLENLIAQLPGEKKNGLFGNLIALSLGLTPNFKSNFDLAEDAKL | |
| wherein X is | QLSKDTYDDDLDNLLAQIGDQYADLFLAAKNLSDAILLSDILRVNTEITKAPL | |
| any alternate | SASMIKRYDEHHQDLTLLKALVRQQLPEKYKEIFFDQSKNGYAGYIDGGASQE | |
| amino acid | EFYKFIKPILEKMDGTEELLVKLNREDLLRKQRTFDNGSIPHQIHLGELHAIL | |
| | RRQEDFYPFLKDNREKIEKILTFRIPYYVGPLARGNSRFAWMTRKSEETITPW | |
| | NFEEVVDKGASAQSFIERMTNFDKNLPNEKVLPKHSLLYEYFTVYNELTKVK | |
| | YVTEGMRKPAFLSGEQKKAIVDLLFKTNRKVTVKQLKEDYFKKIECFDSVEI | |
| | SGVEDRFNASLGTYHDLLKIIKDKDFLDNEENEDILEDIVLTLTLFEDREMIE | |
| | ERLKTYAHLFDDKVMKQLKRRRYTGWGRLSRKLINGIRDKQSGKTILDFLKS | |
| | DGFANRNFMQLIHDDSLTFKEDIQKAQVSGQGDSLHEHIANLAGSPAIKKGI | |
| | LQTVKVVDELVKVMGRHKPENIVIXMARENQTTQKGQKNSRERMKRIEEGIK | |
| | ELGSQILKEHPVENTQLQNEKLYLYYLQNGRDMYVDQELDINRLSDYDVDH | |
| | IVPQSFLKDDSIDNKVLTRSDKNRGKSDNVPSEEVVKKMKNYWRQLLNAKL | |
| | ITQRKFDNLTKAERGGLSELDKAGFIKRQLVETRQITKHVAQILDSRMNTKY | |
| | DENDKLIREVKVITLKSKLVSDFRKDFQFYKVREINNYHHAHDAYLNAVVG | |
| | TALIKKYPKLESEFVYGDYKVYDVRKMIAKSEQEIGKATAKYFFYSNIMNFF | |
| | KTEITLANGEIRKRPLIETNGETGEIVWDKGRDFATVRKVLSMPQVNIVKKTE | |
| | VQTGGFSKESILPKRNSDKLIARKKDWDPKKYGGFDSPTVAYSVLVVAKVEK | |
| | GKSKKLKSVKELLGITIMERSSFEKNPIDFLEAKGYKEVKKDLIIKLPKYSL | |
| | FELENGRKRMLASAGELQKGNELALPSKYVNFLYLASHYEKLKGSPEDNEQK | |
| | QLFVEQHKHYLDEIIEQISEFSKRVILADANLDKVLSAYNKHRDKPIREQAE | |
| | NIIHLFTLTNLGAPAAFKYFDTTIDRKRYTSTKEVLDATLIHQSITGLYETR | |
| | IDLSQLGGD | |
| Cas9 nickase | MDKKYSIGLDIGTNSVGWAVITDEYKVPSKKFKVLGNTDRHSIKKNLIGALL | 83 |
| Streptococcus | FDSGETAEATRLKRTARRRYTRRKNRICYLQEIFSNEMAKVDDSFFHRLEESF | |
| pyogenes | LVEEDKKHERHPIFGNIVDEVAYHEKYPTIYHLRKKLVDSTDKADLRLIYLAL | |
| Q99ZW2 Cas9 | AHMIKFRGHFLIEGDLNPDNSDVDKLFIQLVQTYNQLFEENPINASGVDAKAI | |
| with H983X, | LSARLSKSRRLENLIAQLPGEKKNGLFGNLIALSLGLTPNFKSNFDLAEDAKL | |
| wherein X is | QLSKDTYDDDLDNLLAQIGDQYADLFLAAKNLSDAILLSDILRVNTEITKAPL | |
| any alternate | SASMIKRYDEHHQDLTLLKALVRQQLPEKYKEIFFDQSKNGYAGYIDGGASQE | |
| amino acid | EFYKFIKPILEKMDGTEELLVKLNREDLLRKQRTFDNGSIPHQIHLGELHAIL | |
| | RRQEDFYPFLKDNREKIEKILTFRIPYYVGPLARGNSRFAWMTRKSEETITPW | |
| | NFEEVVDKGASAQSFIERMTNFDKNLPNEKVLPKHSLLYEYFTVYNELTKVKY | |
| | VTEGMRKPAFLSGEQKKAIVDLLFKTNRKVTVKQLKEDYFKKIECFDSVEISG | |
| | VEDRFNASLGTYHDLLKIIKDKDFLDNEENEDILEDIVLTLTLFEDREMIEE | |
| | RLKTYAHLFDDKVMKQLKRRRYTGWGRLSRKLINGIRDKQSGKTILDFLKS | |
| | DGFANRNFMQLIHDDSLTFKEDIQKAQVSGQGDSLHEHIANLAGSPAIKKGIL | |
| | QTVKVVDELVKVMGRHKPENIVIEMARENQTTQKGQKNSRERMKRIEEGIK | |
| | ELGSQILKEHPVENTQLQNEKLYLYYLQNGRDMYVDQELDINRLSDYDVDH | |
| | IVPQSFLKDDSIDNKVLTRSDKNRGKSDNVPSEEVVKKMKNYWRQLLNAKL | |
| | ITQRKFDNLTKAERGGLSELDKAGFIKRQLVETRQITKHVAQILDSRMNTKY | |
| | DENDKLIREVKVITLKSKLVSDFRKDFQFYKVREINNYHXAHDAYLNAVVG | |
| | TALIKKYPKLESEFVYGDYKVYDVRKMIAKSEQEIGKATAKYFFYSNIMNFF | |
| | KTEITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAKVE | |
| | KGKSKKLKSVKELLGITIMERSSFEKNPIDFLEAKGYKEVKKDLIIKLPKYSL | |
| | FELENGRKRMLASAGELQKGNELALPSKYVNFLYLASHYEKLKGSPEDNEQK | |
| | QLFVEQHKHYLDEIIEQISEFSKRVILADANLDKVLSAYNKHRDKPIREQAENI | |
| | IHLFTLTNLGAPAAFKYFDTTIDRKRYTSTKEVLDATLIHQSITGLYETRIDLS | |
| | QLGGD | |
| Cas9 nickase | MDKKYSIGLDIGTNSVGWAVITDEYKVPSKKFKVLGNTDRHSIKKNLIGALL | 84 |
| Streptococcus | FDSGETAEATRLKRTARRRYTRRKNRICYLQEIFSNEMAKVDDSFFHRLEESF | |
| pyogenes | LVEEDKKHERHPIFGNIVDEVAYHEKYPTIYHLRKKLVDSTDKADLRLIYLAL | |
| Q99ZW2 Cas9 | AHMIKFRGHFLIEGDLNPDNSDVDKLFIQLVQTYNQLFEENPINASGVDAKAI | |
| with D986X, | LSARLSKSRRLENLIAQLPGEKKNGLFGNLIALSLGLTPNFKSNFDLAEDAKL | |
| wherein X is | QLSKDTYDDDLDNLLAQIGDQYADLFLAAKNLSDAILLSDILRVNTEITKAPL | |
| any alternate | SASMIKRYDEHHQDLTLLKALVRQQLPEKYKEIFFDQSKNGYAGYIDGGASQ | |
| amino acid | EEFYKFIKPILEKMDGTEELLVKLNREDLLRKQRTFDNGSIPHQIHLGELHAIL | |
| | RRQEDFYPFLKDNREKIEKILTFRIPYYVGPLARGNSRFAWMTRKSEETITPW | |
| | NFEEVVDKGASAQSFIERMTNFDKNLPNEKVLPKHSLLYEYFTVYNELTKVK | |
| | YVTEGMRKPAFLSGEQKKAIVDLLFKTNRKVTVKQLKEDYFKKIECFDSVEI | |
| | SGVEDRFNASLGTYHDLLKIIKDKDFLDNEENEDILEDIVLTLTLFEDREMIEE | |
| | RLKTYAHLFDDKVMKQLKRRRYTGWGRLSRKLINGIRDKQSGKTILDFLKS | |
| | DGFANRNFMQLIHDDSLTFKEDIQKAQVSGQGDSLHEHIANLAGSPAIKKGIL | |
| | QTVKVVDELVKVMGRHKPENIVIEMARENQTTQKGQKNSRERMKRIEEGIK | |
| | ELGSQILKEHPVENTQLQNEKLYLYYLQNGRDMYVDQELDINRLSDYDVDH | |
| | IVPQSFLKDDSIDNKVLTRSDKNRGKSDNVPSEEVVKKMKNYWRQLLNAKL | |
| | ITQRKFDNLTKAERGGLSELDKAGFIKRQLVETRQITKHVAQILDSRMNTKY | |
| | DENDKLIREVKVITLKSKLVSDFRKDFQFYKVREINNYHHAHXAYLNAVVG | |
| | TALIKKYPKLESEFVYGDYKVYDVRKMIAKSEQEIGKATAKYFFYSNIMNFF | |
| | KTEITLANGEIRKRPLIETNGETGEIVWDKGRDFATVRKVLSMPQVNIVKKTE | |
| | VQTGGFSKESILPKRNSDKLIARKKDWDPKKYGGFDSPTVAYSVLVVAKVEK | |
| | GKSKKLKSVKELLGITIMERSSFEKNPIDFLEAKGYKEVKKDLIIKLPKYSL | |
| | FELENGRKRMLASAGELQKGNELALPSKYVNFLYLASHYEKLKGSPEDNEQK | |
| | QLFVEQHKHYLDEIIEQISEFSKRVILADANLDKVLSAYNKHRDKPIREQAE | |
| | NIIHLFTLTNLGAPAAFKYFDTTIDRKRYTSTKEVLDATLIHQSITGLYETR | |
| | IDLSQLGGD | |
| Cas9 nickase | MDKKYSIGLAIGTNSVGWAVITDEYKVPSKKFKVLGNTDRHSIKKNLIGALL | 85 |
| Streptococcus | FDSGETAEATRLKRTARRRYTRRKNRICYLQEIFSNEMAKVDDSFFHRLEESF | |
| pyogenes | LVEEDKKHERHPIFGNIVDEVAYHEKYPTIYHLRKKLVDSTDKADLRLIYLAL | |
| Q99ZW2 Cas9 | AHMIKFRGHFLIEGDLNPDNSDVDKLFIQLVQTYNQLFEENPINASGVDAKAI | |
| with D10A | LSARLSKSRRLENLIAQLPGEKKNGLFGNLIALSLGLTPNFKSNFDLAEDAKL | |
| | QLSKDTYDDDLDNLLAQIGDQYADLFLAAKNLSDAILLSDILRVNTEITKAPL | |
| | SASMIKRYDEHHQDLTLLKALVRQQLPEKYKEIFFDQSKNGYAGYIDGGASQE | |
| | EFYKFIKPILEKMDGTEELLVKLNREDLLRKQRTFDNGSIPHQIHLGELHAIL | |
| | RRQEDFYPFLKDNREKIEKILTFRIPYYVGPLARGNSRFAWMTRKSEETITPW | |
| | NFEEVVDKGASAQSFIERMTNFDKNLPNEKVLPKHSLLYEYFTVYNELTKVKY | |
| | VTEGMRKPAFLSGEQKKAIVDLLFKTNRKVTVKQLKEDYFKKIECFDSVEISG | |
| | VEDRFNASLGTYHDLLKIIKDKDFLDNEENEDILEDIVLTLTLFEDREMIEE | |
| | RLKTYAHLFDDKVMKQLKRRRYTGWGRLSRKLINGIRDKQSGKTILDFLKSD | |
| | GFANRNFMQLIHDDSLTFKEDIQKAQVSGQGDSLHEHIANLAGSPAIKKGIL | |
| | QTVKVVDELVKVMGRHKPENIVIEMARENQTTQKGQKNSRERMKRIEEGIK | |
| | ELGSQILKEHPVENTQLQNEKLYLYYLQNGRDMYVDQELDINRLSDYDVDH | |
| | IVPQSFLKDDSIDNKVLTRSDKNRGKSDNVPSEEVVKKMKNYWRQLLNAKL | |
| | ITQRKFDNLTKAERGGLSELDKAGFIKRQLVETRQITKHVAQILDSRMNTKY | |
| | DENDKLIREVKVITLKSKLVSDFRKDFQFYKVREINNYHHAHDAYLNAVVG | |
| | TALIKKYPKLESEFVYGDYKVYDVRKMIAKSEQEIGKATAKYFFYSNIMNFF | |
| | KTEITLANGEIRKRPLIETNGETGEIVWDKGRDFATVRKVLSMPQVNIVKKTE | |
| | VQTGGFSKESILPKRNSDKLIARKKDWDPKKYGGFDSPTVAYSVLVVAKVEK | |
| | GKSKKLKSVKELLGITIMERSSFEKNPIDFLEAKGYKEVKKDLIIKLPKYSL | |
| | FELENGRKRMLASAGELQKGNELALPSKYVNFLYLASHYEKLKGSPEDNEQK | |
| | QLFVEQHKHYLDEIIEQISEFSKRVILADANLDKVLSAYNKHRDKPIREQAE | |
| | NIIHLFTLTNLGAPAAFKYFDTTIDRKRYTSTKEVLDATLIHQSITGLYETR | |
| | IDLSQLGGD | |
| Cas9 nickase | MDKKYSIGLDIGTNSVGWAVITDEYKVPSKKFKVLGNTDRHSIKKNLIGALL | 86 |
| Streptococcus | FDSGETAEATRLKRTARRRYTRRKNRICYLQEIFSNEMAKVDDSFFHRLEESF | |
| pyogenes | LVEEDKKHERHPIFGNIVDEVAYHEKYPTIYHLRKKLVDSTDKADLRLIYLAL | |
| Q99ZW2 Cas9 | AHMIKFRGHFLIEGDLNPDNSDVDKLFIQLVQTYNQLFEENPINASGVDAKAI | |
| with E762A | LSARLSKSRRLENLIAQLPGEKKNGLFGNLIALSLGLTPNFKSNFDLAEDAKL | |
| | QLSKDTYDDDLDNLLAQIGDQYADLFLAAKNLSDAILLSDILRVNTEITKAPL | |
| | SASMIKRYDEHHQDLTLLKALVRQQLPEKYKEIFFDQSKNGYAGYIDGGASQ | |
| | EEFYKFIKPILEKMDGTEELLVKLNREDLLRKQRTFDNGSIPHQIHLGELHAIL | |
| | RRQEDFYPFLKDNREKIEKILTFRIPYYVGPLARGNSRFAWMTRKSEETITPW | |
| | NFEEVVDKGASAQSFIERMTNFDKNLPNEKVLPKHSLLYEYFTVYNELTKVK | |
| | YVTEGMRKPAFLSGEQKKAIVDLLFKTNRKVTVKQLKEDYFKKIECFDSVEI | |
| | SGVEDRFNASLGTYHDLLKIIKDKDFLDNEENEDILEDIVLTLTLFEDREMIE | |
| | ERLKTYAHLFDDKVMKQLKRRRYTGWGRLSRKLINGIRDKQSGKTILDFLKS | |
| | DGFANRNFMQLIHDDSLTFKEDIQKAQVSGQGDSLHEHIANLAGSPAIKKGI | |
| | LQTVKVVDELVKVMGRHKPENIVIAMARENQTTQKGQKNSRERMKRIEEGIK | |
| | ELGSQILKEHPVENTQLQNEKLYLYYLQNGRDMYVDQELDINRLSDYDVDH | |
| | IVPQSFLKDDSIDNKVLTRSDKNRGKSDNVPSEEVVKKMKNYWRQLLNAKL | |
| | ITQRKFDNLTKAERGGLSELDKAGFIKRQLVETRQITKHVAQILDSRMNTKY | |
| | DENDKLIREVKVITLKSKLVSDFRKDFQFYKVREINNYHHAHDAYLNAVVG | |
| | TALIKKYPKLESEFVYGDYKVYDVRKMIAKSEQEIGKATAKYFFYSNIMNFF | |
| | KTEITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAKVE | |
| | KGKSKKLKSVKELLGITIMERSSFEKNPIDFLEAKGYKEVKKDLIIKLPKYS | |
| | LFELENGRKRMLASAGELQKGNELALPSKYVNFLYLASHYEKLKGSPEDNEQ | |
| | KQLFVEQHKHYLDEIIEQISEFSKRVILADANLDKVLSAYNKHRDKPIREQA | |
| | ENIIHLFTLTNLGAPAAFKYFDTTIDRKRYTSTKEVLDATLIHQSITGLYET | |
| | RIDLSQLGGD | |
| Cas9 nickase | MDKKYSIGLDIGTNSVGWAVITDEYKVPSKKFKVLGNTDRHSIKKNLIGALL | 87 |
| Streptococcus | FDSGETAEATRLKRTARRRYTRRKNRICYLQEIFSNEMAKVDDSFFHRLEESF | |
| pyogenes | LVEEDKKHERHPIFGNIVDEVAYHEKYPTIYHLRKKLVDSTDKADLRLIYLAL | |
| Q99ZW2 Cas9 | AHMIKFRGHFLIEGDLNPDNSDVDKLFIQLVQTYNQLFEENPINASGVDAKAI | |
| with H983A | LSARLSKSRRLENLIAQLPGEKKNGLFGNLIALSLGLTPNFKSNFDLAEDAKL | |
| | QLSKDTYDDDLDNLLAQIGDQYADLFLAAKNLSDAILLSDILRVNTEITKAPL | |
| | SASMIKRYDEHHQDLTLLKALVRQQLPEKYKEIFFDQSKNGYAGYIDGGASQ | |
| | EEFYKFIKPILEKMDGTEELLVKLNREDLLRKQRTFDNGSIPHQIHLGELHAIL | |
| | RRQEDFYPFLKDNREKIEKILTFRIPYYVGPLARGNSRFAWMTRKSEETITPW | |
| | NFEEVVDKGASAQSFIERMTNFDKNLPNEKVLPKHSLLYEYFTVYNELTKVK | |
| | YVTEGMRKPAFLSGEQKKAIVDLLFKTNRKVTVKQLKEDYFKKIECFDSVEIS | |
| | GVEDRFNASLGTYHDLLKIIKDKDFLDNEENEDILEDIVLTLTLFEDREMIEE | |
| | RLKTYAHLFDDKVMKQLKRRRYTGWGRLSRKLINGIRDKQSGKTILDFLKSD | |
| | GFANRNFMQLIHDDSLTFKEDIQKAQVSGQGDSLHEHIANLAGSPAIKKGIL | |
| | QTVKVVDELVKVMGRHKPENIVIEMARENQTTQKGQKNSRERMKRIEEGIK | |
| | ELGSQILKEHPVENTQLQNEKLYLYYLQNGRDMYVDQELDINRLSDYDVDH | |
| | IVPQSFLKDDSIDNKVLTRSDKNRGKSDNVPSEEVVKKMKNYWRQLLNAKL | |
| | ITQRKFDNLTKAERGGLSELDKAGFIKRQLVETRQITKHVAQILDSRMNTKY | |
| | DENDKLIREVKVITLKSKLVSDFRKDFQFYKVREINNYHAAHDAYLNAVVG | |
| | TALIKKYPKLESEFVYGDYKVYDVRKMIAKSEQEIGKATAKYFFYSNIMNFF | |
| | KTEITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAKVE | |
| | KGKSKKLKSVKELLGITIMERSSFEKNPIDFLEAKGYKEVKKDLIIKLPKYSL | |
| | FELENGRKRMLASAGELQKGNELALPSKYVNFLYLASHYEKLKGSPEDNEQKQ | |
| | LFVEQHKHYLDEIIEQISEFSKRVILADANLDKVLSAYNKHRDKPIREQAENI | |
| | IHLFTLTNLGAPAAFKYFDTTIDRKRYTSTKEVLDATLIHQSITGLYETRIDL | |
| | SQLGGD | |
| Cas9 nickase | MDKKYSIGLDIGTNSVGWAVITDEYKVPSKKFKVLGNTDRHSIKKNLIGALL | 88 |
| Streptococcus | FDSGETAEATRLKRTARRRYTRRKNRICYLQEIFSNEMAKVDDSFFHRLEESF | |
| pyogenes | LVEEDKKHERHPIFGNIVDEVAYHEKYPTIYHLRKKLVDSTDKADLRLIYLAL | |
| Q99ZW2 Cas9 | AHMIKFRGHFLIEGDLNPDNSDVDKLFIQLVQTYNQLFEENPINASGVDAKAI | |
| with D986A | LSARLSKSRRLENLIAQLPGEKKNGLFGNLIALSLGLTPNFKSNFDLAEDAKL | |
| | QLSKDTYDDDLDNLLAQIGDQYADLFLAAKNLSDAILLSDILRVNTEITKAPL | |
| | SASMIKRYDEHHQDLTLLKALVRQQLPEKYKEIFFDQSKNGYAGYIDGGASQ | |
| | EEFYKFIKPILEKMDGTEELLVKLNREDLLRKQRTFDNGSIPHQIHLGELHAIL | |
| | RRQEDFYPFLKDNREKIEKILTFRIPYYVGPLARGNSRFAWMTRKSEETITPW | |
| | NFEEVVDKGASAQSFIERMTNFDKNLPNEKVLPKHSLLYEYFTVYNELTKVK | |
| | YVTEGMRKPAFLSGEQKKAIVDLLFKTNRKVTVKQLKEDYFKKIECFDSVEIS | |
| | GVEDRFNASLGTYHDLLKIIKDKDFLDNEENEDILEDIVLTLTLFEDREMIEE | |
| | RLKTYAHLFDDKVMKQLKRRRYTGWGRLSRKLINGIRDKQSGKTILDFLKSD | |
| | GFANRNFMQLIHDDSLTFKEDIQKAQVSGQGDSLHEHIANLAGSPAIKKGIL | |
| | QTVKVVDELVKVMGRHKPENIVIEMARENQTTQKGQKNSRERMKRIEEGIK | |
| | ELGSQILKEHPVENTQLQNEKLYLYYLQNGRDMYVDQELDINRLSDYDVDH | |
| | IVPQSFLKDDSIDNKVLTRSDKNRGKSDNVPSEEVVKKMKNYWRQLLNAKL | |
| | ITQRKFDNLTKAERGGLSELDKAGFIKRQLVETRQITKHVAQILDSRMNTKY | |
| | DENDKLIREVKVITLKSKLVSDFRKDFQFYKVREINNYHHAHAAYLNAVVG | |
| | TALIKKYPKLESEFVYGDYKVYDVRKMIAKSEQEIGKATAKYFFYSNIMNFF | |
| | KTEITLANGEIRKRPLIETNGETGEIVWDKGRDFATVRKVLSMPQVNIVKKTE | |
| | VQTGGFSKESILPKRNSDKLIARKKDWDPKKYGGFDSPTVAYSVLVVAKVEK | |
| | GKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQK | |
| | QLFVEQHKHYLDEIIEQISEFSKRVILADANLDKVLSAYNKHRDKPIREQAENI | |
| | IHLFTLTNLGAPAAFKYFDTTIDRKRYTSTKEVLDATLIHQSITGLYETRIDLS | |
| | QLGGD | |

In another embodiment, the Cas9 nickase comprises a mutation in the HNH domain which inactivates the HNH nuclease activity. For example, mutations in histidine (H) 840 or asparagine (R) 863 have been reported as loss-of-function mutations of the HNH nuclease domain and the creation of a functional Cas9 nickase (e.g., Nishimasu et al., “Crystal structure of Cas9 in complex with guide RNA and target DNA,” Cell 156(5), 935-949, which is incorporated herein by reference). Thus, nickase mutations in the HNH domain could include H840X and R863X, wherein X is any amino acid other than the wild type amino acid. In certain embodiments, the nickase could be H840A or R863A or a combination thereof.

In various embodiments, the Cas9 nickase can have a mutation in the HNH nuclease domain and have one of the following amino acid sequences, or a variant thereof having an amino acid sequence that has at least 80%, at least 85%, at least 90%, at least 95%, or at least 99% sequence identity thereto.

| | | SEQ |
|---|---|---|
| | | ID |
| Description | Sequence | NO: |
| Cas9 nickase | MDKKYSIGLDIGTNSVGWAVITDEYKVPSKKFKVLGNTDRHSIKKNLIGALLF | 89 |
| Streptococcus | DSGETAEATRLKRTARRRYTRRKNRICYLQEIFSNEMAKVDDSFFHRLEESFLV | |
| pyogenes | EEDKKHERHPIFGNIVDEVAYHEKYPTIYHLRKKLVDSTDKADLRLIYLALAH | |
| Q99ZW2 Cas9 | MIKFRGHFLIEGDLNPDNSDVDKLFIQLVQTYNQLFEENPINASGVDAKAILSA | |
| with H840X, | RLSKSRRLENLIAQLPGEKKNGLFGNLIALSLGLTPNFKSNFDLAEDAKLQLSK | |
| wherein X is | DTYDDDLDNLLAQIGDQYADLFLAAKNLSDAILLSDILRVNTEITKAPLSASMI | |
| any alternate | KRYDEHHQDLTLLKALVRQQLPEKYKEIFFDQSKNGYAGYIDGGASQEEFYKF | |
| amino acid | IKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDK | |
| | GASAQSFIERMTNFDKNLPNEKVLPKHSLLYEYFTVYNELTKVKYVTEGMRKP | |
| | AFLSGEQKKAIVDLLFKTNRKVTVKQLKEDYFKKIECFDSVEISGVEDRFNASL | |
| | GTYHDLLKIIKDKDFLDNEENEDILEDIVLTLTLFEDREMIEERLKTYAHLFDDK | |
| | VMKQLKRRRYTGWGRLSRKLINGIRDKQSGKTILDFLKSDGFANRNFMQLIHD | |
| | DSLTFKEDIQKAQVSGQGDSLHEHIANLAGSPAIKKGILQTVKVVDELVKVMG | |
| | RHKPENIVIEMARENQTTQKGQKNSRERMKRIEEGIKELGSQILKEHPVENTQL | |
| | QNEKLYLYYLQNGRDMYVDQELDINRLSDYDVDXIVPQSFLKDDSIDNKVLTR | |
| | SDKNRGKSDNVPSEEVVKKMKNYWRQLLNAKLITQRKFDNLTKAERGGLSEL | |
| | DKAGFIKRQLVETRQITKHVAQILDSRMNTKYDENDKLIREVKVITLKSKLVSD | |
| | FRKDFQFYKVREINNYHHAHDAYLNAVVGTALIKKYPKLESEFVYGDYKVYDV | |
| | RKMIAKSEQEIGKATAKYFFYSNIMNFFKTEITLANGEIRKRPLIETNGETGEI | |
| | VWDKGRDFATVRKVLSMPQVNIVKKTEVQTGGFSKESILPKRNSDKLIARKKD | |
| | WDPKKYGGFDSPTVAYSVLVVAKVEKGKSKKLKSVKELLGITIMERSSFEKNP | |
| | IDFLEAKGYKEVKKDLIIKLPKYSLFELENGRKRMLASAGELQKGNELALPSKY | |
| | VNFLYLASHYEKLKGSPEDNEQKQLFVEQHKHYLDEIIEQISEFSKRVILADAN | |
| | LDKVLSAYNKHRDKPIREQAENIIHLFTLTNLGAPAAFKYFDTTIDRKRYTSTK | |
| | EVLDATLIHQSITGLYETRIDLSQLGGD | |
| Cas9 nickase | MDKKYSIGLDIGTNSVGWAVITDEYKVPSKKFKVLGNTDRHSIKKNLIGALLF | 90 |
| Streptococcus | DSGETAEATRLKRTARRRYTRRKNRICYLQEIFSNEMAKVDDSFFHRLEESFLV | |
| pyogenes | EEDKKHERHPIFGNIVDEVAYHEKYPTIYHLRKKLVDSTDKADLRLIYLALAH | |
| Q99ZW2 Cas9 | MIKFRGHFLIEGDLNPDNSDVDKLFIQLVQTYNQLFEENPINASGVDAKAILSA | |
| with H840A, | RLSKSRRLENLIAQLPGEKKNGLFGNLIALSLGLTPNFKSNFDLAEDAKLQLSK | |
| wherein X is | DTYDDDLDNLLAQIGDQYADLFLAAKNLSDAILLSDILRVNTEITKAPLSASMI | |
| any alternate | KRYDEHHQDLTLLKALVRQQLPEKYKEIFFDQSKNGYAGYIDGGASQEEFYKF | |
| amino acid | IKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDK | |
| | GASAQSFIERMTNFDKNLPNEKVLPKHSLLYEYFTVYNELTKVKYVTEGMRKP | |
| | AFLSGEQKKAIVDLLFKTNRKVTVKQLKEDYFKKIECFDSVEISGVEDRFNASL | |
| | GTYHDLLKIIKDKDFLDNEENEDILEDIVLTLTLFEDREMIEERLKTYAHLFDDK | |
| | VMKQLKRRRYTGWGRLSRKLINGIRDKQSGKTILDFLKSDGFANRNFMQLIHD | |
| | DSLTFKEDIQKAQVSGQGDSLHEHIANLAGSPAIKKGILQTVKVVDELVKVMG | |
| | RHKPENIVIEMARENQTTQKGQKNSRERMKRIEEGIKELGSQILKEHPVENTQL | |
| | QNEKLYLYYLQNGRDMYVDQELDINRLSDYDVDAIVPQSFLKDDSIDNKVLTR | |
| | SDKNRGKSDNVPSEEVVKKMKNYWRQLLNAKLITQRKFDNLTKAERGGLSEL | |
| | DKAGFIKRQLVETRQITKHVAQILDSRMNTKYDENDKLIREVKVITLKSKLVSD | |
| | FRKDFQFYKVREINNYHHAHDAYLNAVVGTALIKKYPKLESEFVYGDYKVYDV | |
| | RKMIAKSEQEIGKATAKYFFYSNIMNFFKTEITLANGEIRKRPLIETNGETGEI | |
| | VWDKGRDFATVRKVLSMPQVNIVKKTEVQTGGFSKESILPKRNSDKLIARKKD | |
| | WDPKKYGGFDSPTVAYSVLVVAKVEKGKSKKLKSVKELLGITIMERSSFEKNP | |
| | IDFLEAKGYKEVKKDLIIKLPKYSLFELENGRKRMLASAGELQKGNELALPSKY | |
| | VNFLYLASHYEKLKGSPEDNEQKQLFVEQHKHYLDEIIEQISEFSKRVILADAN | |
| | LDKVLSAYNKHRDKPIREQAENIIHLFTLTNLGAPAAFKYFDTTIDRKRYTSTK | |
| | EVLDATLIHQSITGLYETRIDLSQLGGD | |
| Cas9 nickase | MDKKYSIGLDIGTNSVGWAVITDEYKVPSKKFKVLGNTDRHSIKKNLIGALLF | 91 |
| Q99ZW2 Cas9 | DSGETAEATRLKRTARRRYTRRKNRICYLQEIFSNEMAKVDDSFFHRLEESFLV | |
| Streptococcus | MIKFRGHFLIEGDLNPDNSDVDKLFIQLVQTYNQLFEENPINASGVDAKAILSA | |
| pyogenes | EEDKKHERHPIFGNIVDEVAYHEKYPTIYHLRKKLVDSTDKADLRLIYLALAH | |
| with R863X, | RLSKSRRLENLIAQLPGEKKNGLFGNLIALSLGLTPNFKSNFDLAEDAKLQLSK | |
| wherein X is | DTYDDDLDNLLAQIGDQYADLFLAAKNLSDAILLSDILRVNTEITKAPLSASMI | |
| any alternate | KRYDEHHQDLTLLKALVRQQLPEKYKEIFFDQSKNGYAGYIDGGASQEEFYKF | |
| amino acid | IKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDK | |
| | GASAQSFIERMTNFDKNLPNEKVLPKHSLLYEYFTVYNELTKVKYVTEGMRKP | |
| | AFLSGEQKKAIVDLLFKTNRKVTVKQLKEDYFKKIECFDSVEISGVEDRFNASL | |
| | GTYHDLLKIIKDKDFLDNEENEDILEDIVLTLTLFEDREMIEERLKTYAHLFDDK | |
| | VMKQLKRRRYTGWGRLSRKLINGIRDKQSGKTILDFLKSDGFANRNFMQLIHD | |
| | DSLTFKEDIQKAQVSGQGDSLHEHIANLAGSPAIKKGILQTVKVVDELVKVMG | |
| | RHKPENIVIEMARENQTTQKGQKNSRERMKRIEEGIKELGSQILKEHPVENTQL | |
| | QNEKLYLYYLQNGRDMYVDQELDINRLSDYDVDHIVPQSFLKDDSIDNKVLTR | |
| | SDKNXGKSDNVPSEEVVKKMKNYWRQLLNAKLITQRKFDNLTKAERGGLSEL | |
| | DKAGFIKRQLVETRQITKHVAQILDSRMNTKYDENDKLIREVKVITLKSKLVSD | |
| | FRKDFQFYKVREINNYHHAHDAYLNAVVGTALIKKYPKLESEFVYGDYKVYD | |
| | VRKMIAKSEQEIGKATAKYFFYSNIMNFFKTEITLANGEIRKRPLIETNGETGEIV | |
| | WDKGRDFATVRKVLSMPQVNIVKKTEVQTGGFSKESILPKRNSDKLIARKKD | |
| | WDPKKYGGFDSPTVAYSVLVVAKVEKGKSKKLKSVKELLGITIMERSSFEKNP | |
| | IDFLEAKGYKEVKKDLIIKLPKYSLFELENGRKRMLASAGELQKGNELALPSKY | |
| | VNFLYLASHYEKLKGSPEDNEQKQLFVEQHKHYLDEIIEQISEFSKRVILADAN | |
| | LDKVLSAYNKHRDKPIREQAENIIHLFTLTNLGAPAAFKYFDTTIDRKRYTSTK | |
| | EVLDATLIHQSITGLYETRIDLSQLGGD | |
| Cas9 nickase | MDKKYSIGLDIGTNSVGWAVITDEYKVPSKKFKVLGNTDRHSIKKNLIGALLF | 92 |
| Streptococcus | DSGETAEATRLKRTARRRYTRRKNRICYLQEIFSNEMAKVDDSFFHRLEESFLV | |
| pyogenes | EEDKKHERHPIFGNIVDEVAYHEKYPTIYHLRKKLVDSTDKADLRLIYLALAH | |
| Q99ZW2 Cas9 | MIKFRGHFLIEGDLNPDNSDVDKLFIQLVQTYNQLFEENPINASGVDAKAILSA | |
| with R863A, | RLSKSRRLENLIAQLPGEKKNGLFGNLIALSLGLTPNFKSNFDLAEDAKLQLSK | |
| wherein X is | DTYDDDLDNLLAQIGDQYADLFLAAKNLSDAILLSDILRVNTEITKAPLSASMI | |
| any alternate | KRYDEHHQDLTLLKALVRQQLPEKYKEIFFDQSKNGYAGYIDGGASQEEFYKFI | |
| amino acid | KPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDK | |
| | GASAQSFIERMTNFDKNLPNEKVLPKHSLLYEYFTVYNELTKVKYVTEGMRKP | |
| | AFLSGEQKKAIVDLLFKTNRKVTVKQLKEDYFKKIECFDSVEISGVEDRFNASL | |
| | GTYHDLLKIIKDKDFLDNEENEDILEDIVLTLTLFEDREMIEERLKTYAHLFDD | |
| | KVMKQLKRRRYTGWGRLSRKLINGIRDKQSGKTILDFLKSDGFANRNFMQLIHD | |
| | DSLTFKEDIQKAQVSGQGDSLHEHIANLAGSPAIKKGILQTVKVVDELVKVMG | |
| | RHKPENIVIEMARENQTTQKGQKNSRERMKRIEEGIKELGSQILKEHPVENTQL | |
| | QNEKLYLYYLQNGRDMYVDQELDINRLSDYDVDHIVPQSFLKDDSIDNKVLTR | |
| | SDKNAGKSDNVPSEEVVKKMKNYWRQLLNAKLITQRKFDNLTKAERGGLSEL | |
| | DKAGFIKRQLVETRQITKHVAQILDSRMNTKYDENDKLIREVKVITLKSKLVSD | |
| | FRKDFQFYKVREINNYHHAHDAYLNAVVGTALIKKYPKLESEFVYGDYKVYD | |
| | VRKMIAKSEQEIGKATAKYFFYSNIMNFFKTEITLANGEIRKRPLIETNGETGE | |
| | IVWDKGRDFATVRKVLSMPQVNIVKKTEVQTGGFSKESILPKRNSDKLIARKKD | |
| | WDPKKYGGFDSPTVAYSVLVVAKVEKGKSKKLKSVKELLGITIMERSSFEKNP | |
| | IDFLEAKGYKEVKKDLIIKLPKYSLFELENGRKRMLASAGELQKGNELALPSKY | |
| | VNFLYLASHYEKLKGSPEDNEQKQLFVEQHKHYLDEIIEQISEFSKRVILADAN | |
| | LDKVLSAYNKHRDKPIREQAENIIHLFTLTNLGAPAAFKYFDTTIDRKRYTSTK | |
| | EVLDATLIHQSITGLYETRIDLSQLGGD | |

In some embodiments, the N-terminal methionine is removed from a Cas9 nickase, or from any Cas9 variant, ortholog, or equivalent disclosed or contemplated herein. For example, methionine-minus Cas9 nickases include the following sequences, or a variant thereof having an amino acid sequence that has at least 80%, at least 85%, at least 90%, at least 95%, or at least 99% sequence identity thereto.

| Description | Sequence |
|---|---|
| Cas9 nickase | DKKYSIGLDIGTNSVGWAVITDEYKVPSKKFKVLGNTDRHSIKKNLIGALLFDSGET |
| (Met minus) | AEATRLKRTARRRYTRRKNRICYLQEIFSNEMAKVDDSFFHRLEESFLVEEDKKHER |
| Streptococcus | HPIFGNIVDEVAYHEKYPTIYHLRKKLVDSTDKADLRLIYLALAHMIKFRGHFLIEGD |
| pyogenes | LNPDNSDVDKLFIQLVQTYNQLFEENPINASGVDAKAILSARLSKSRRLENLIAQLPG |
| Q99ZW2 Cas9 | EKKNGLFGNLIALSLGLTPNFKSNFDLAEDAKLQLSKDTYDDDLDNLLAQIGDQYA |
| with H840X, | DLFLAAKNLSDAILLSDILRVNTEITKAPLSASMIKRYDEHHQDLTLLKALVRQQLPE |
| wherein X is | KYKEIFFDQSKNGYAGYIDGGASQEEFYKFIKPILEKMDGTEELLVKLNREDLLRKQ |
| any alternate | RTFDNGSIPHQIHLGELHAILRRQEDFYPFLKDNREKIEKILTFRIPYYVGPLARGNS |
| amino acid | RFAWMTRKSEETITPWNFEEVVDKGASAQSFIERMTNFDKNLPNEKVLPKHSLLYEY |
| | FTVYNELTKVKYVTEGMRKPAFLSGEQKKAIVDLLFKTNRKVTVKQLKEDYFKKIE |
| | CFDSVEISGVEDRFNASLGTYHDLLKIIKDKDFLDNEENEDILEDIVLTLTLFEDREMI |
| | EERLKTYAHLFDDKVMKQLKRRRYTGWGRLSRKLINGIRDKQSGKTILDFLKSDGF |
| | ANRNFMQLIHDDSLTFKEDIQKAQVSGQGDSLHEHIANLAGSPAIKKGILQTVKVVD |
| | ELVKVMGRHKPENIVIEMARENQTTQKGQKNSRERMKRIEEGIKELGSQILKEHPVE |
| | NTQLQNEKLYLYYLQNGRDMYVDQELDINRLSDYDVDXIVPQSFLKDDSIDNKVLT |
| | RSDKNRGKSDNVPSEEVVKKMKNYWRQLLNAKLITQRKFDNLTKAERGGLSELDK |
| | AGFIKRQLVETRQITKHVAQILDSRMNTKYDENDKLIREVKVITLKSKLVSDFRKDF |
| | QFYKVREINNYHHAHDAYLNAVVGTALIKKYPKLESEFVYGDYKVYDVRKMIAKS |
| | EQEIGKATAKYFFYSNIMNFFKTEITLANGEIRKRPLIETNGETGEIVWDKGRDFATV |
| | RKVLSMPQVNIVKKTEVQTGGFSKESILPKRNSDKLIARKKDWDPKKYGGFDSPTV |
| | AYSVLVVAKVEKGKSKKLKSVKELLGITIMERSSFEKNPIDFLEAKGYKEVKKDLIIK |
| | LPKYSLFELENGRKRMLASAGELQKGNELALPSKYVNFLYLASHYEKLKGSPEDNE |
| | QKOLFVEQHKHYLDEIIEQISEFSKRVILADANLDKVLSAYNKHRDKPIREQAENIIH |
| | LFTLTNLGAPAAFKYFDTTIDRKRYTSTKEVLDATLIHQSITGLYETRIDLSQLGGD |
| | (SEQ ID NO: 93) |
| Cas9 nickase | DKKYSIGLDIGTNSVGWAVITDEYKVPSKKFKVLGNTDRHSIKKNLIGALLFDSGET |
| (Met minus) | AEATRLKRTARRRYTRRKNRICYLQEIFSNEMAKVDDSFFHRLEESFLVEEDKKHER |
| Streptococcus | HPIFGNIVDEVAYHEKYPTIYHLRKKLVDSTDKADLRLIYLALAHMIKFRGHFLIEGD |
| pyogenes | LNPDNSDVDKLFIQLVQTYNQLFEENPINASGVDAKAILSARLSKSRRLENLIAQLPG |
| Q99ZW2 Cas9 | EKKNGLFGNLIALSLGLTPNFKSNFDLAEDAKLQLSKDTYDDDLDNLLAQIGDQYA |
| with H840A, | DLFLAAKNLSDAILLSDILRVNTEITKAPLSASMIKRYDEHHQDLTLLKALVRQQLPE |
| wherein X is | KYKEIFFDQSKNGYAGYIDGGASQEEFYKFIKPILEKMDGTEELLVKLNREDLLRKQ |
| any alternate | RTFDNGSIPHQIHLGELHAILRRQEDFYPFLKDNREKIEKILTFRIPYYVGPLARGNSR |
| amino acid | FAWMTRKSEETITPWNFEEVVDKGASAQSFIERMTNFDKNLPNEKVLPKHSLLYEY |
| | FTVYNELTKVKYVTEGMRKPAFLSGEQKKAIVDLLFKTNRKVTVKQLKEDYFKKIE |
| | CFDSVEISGVEDRFNASLGTYHDLLKIIKDKDFLDNEENEDILEDIVLTLTLFEDREMI |
| | EERLKTYAHLFDDKVMKQLKRRRYTGWGRLSRKLINGIRDKQSGKTILDFLKSDGF |
| | ANRNFMQLIHDDSLTFKEDIQKAQVSGQGDSLHEHIANLAGSPAIKKGILQTVKVVD |
| | ELVKVMGRHKPENIVIEMARENQTTQKGQKNSRERMKRIEEGIKELGSQILKEHPVE |
| | NTQLQNEKLYLYYLQNGRDMYVDQELDINRLSDYDVDAIVPQSFLKDDSIDNKVLT |
| | RSDKNRGKSDNVPSEEVVKKMKNYWRQLLNAKLITQRKFDNLTKAERGGLSELDK |
| | AGFIKRQLVETRQITKHVAQILDSRMNTKYDENDKLIREVKVITLKSKLVSDFRKDF |
| | QFYKVREINNYHHAHDAYLNAVVGTALIKKYPKLESEFVYGDYKVYDVRKMIAKS |
| | EQEIGKATAKYFFYSNIMNFFKTEITLANGEIRKRPLIETNGETGEIVWDKGRDFATV |
| | RKVLSMPQVNIVKKTEVQTGGFSKESILPKRNSDKLIARKKDWDPKKYGGFDSPTV |
| | AYSVLVVAKVEKGKSKKLKSVKELLGITIMERSSFEKNPIDFLEAKGYKEVKKDLIIK |
| | LPKYSLFELENGRKRMLASAGELQKGNELALPSKYVNFLYLASHYEKLKGSPEDNE |
| | QKQLFVEQHKHYLDEIIEQISEFSKRVILADANLDKVLSAYNKHRDKPIREQAENIIH |
| | LFTLTNLGAPAAFKYFDTTIDRKRYTSTKEVLDATLIHQSITGLYETRIDLSQLGGD |
| | (SEQ ID NO: 94) |
| Cas9 nickase | DKKYSIGLDIGTNSVGWAVITDEYKVPSKKFKVLGNTDRHSIKKNLIGALLFDSGET |
| (Met minus) | AEATRLKRTARRRYTRRKNRICYLQEIFSNEMAKVDDSFFHRLEESFLVEEDKKHER |
| Streptococcus | HPIFGNIVDEVAYHEKYPTIYHLRKKLVDSTDKADLRLIYLALAHMIKFRGHFLIEGD |
| pyogenes | LNPDNSDVDKLFIQLVQTYNQLFEENPINASGVDAKAILSARLSKSRRLENLIAQLPG |
| Q99ZW2 Cas9 | EKKNGLFGNLIALSLGLTPNFKSNFDLAEDAKLQLSKDTYDDDLDNLLAQIGDQYA |
| with R863X, | DLFLAAKNLSDAILLSDILRVNTEITKAPLSASMIKRYDEHHQDLTLLKALVRQQLPE |
| wherein X is | KYKEIFFDQSKNGYAGYIDGGASQEEFYKFIKPILEKMDGTEELLVKLNREDLLRKQ |
| any | RTFDNGSIPHQIHLGELHAILRRQEDFYPFLKDNREKIEKILTFRIPYYVGPLARGNSR |
| alternate | FAWMTRKSEETITPWNFEEVVDKGASAQSFIERMTNFDKNLPNEKVLPKHSLLYEY |
| amino acid | FTVYNELTKVKYVTEGMRKPAFLSGEQKKAIVDLLFKTNRKVTVKQLKEDYFKKIE |
| | CFDSVEISGVEDRFNASLGTYHDLLKIIKDKDFLDNEENEDILEDIVLTLTLFEDREMI |
| | EERLKTYAHLFDDKVMKQLKRRRYTGWGRLSRKLINGIRDKQSGKTILDFLKSDGF |
| | ANRNFMQLIHDDSLTFKEDIQKAQVSGQGDSLHEHIANLAGSPAIKKGILQTVKVVD |
| | ELVKVMGRHKPENIVIEMARENQTTQKGQKNSRERMKRIEEGIKELGSQILKEHPVE |
| | NTQLQNEKLYLYYLQNGRDMYVDQELDINRLSDYDVDHIVPQSFLKDDSIDNKVLT |
| | RSDKNXGKSDNVPSEEVVKKMKNYWRQLLNAKLITQRKFDNLTKAERGGLSELDK |
| | AGFIKRQLVETRQITKHVAQILDSRMNTKYDENDKLIREVKVITLKSKLVSDFRKDF |
| | QFYKVREINNYHHAHDAYLNAVVGTALIKKYPKLESEFVYGDYKVYDVRKMIAKS |
| | EQEIGKATAKYFFYSNIMNFFKTEITLANGEIRKRPLIETNGETGEIVWDKGRDFATV |
| | RKVLSMPQVNIVKKTEVQTGGFSKESILPKRNSDKLIARKKDWDPKKYGGFDSPTV |
| | AYSVLVVAKVEKGKSKKLKSVKELLGITIMERSSFEKNPIDFLEAKGYKEVKKDLIIK |
| | LPKYSLFELENGRKRMLASAGELQKGNELALPSKYVNFLYLASHYEKLKGSPEDNE |
| | QKQLFVEQHKHYLDEIIEQISEFSKRVILADANLDKVLSAYNKHRDKPIREQAENIIH |
| | LFTLTNLGAPAAFKYFDTTIDRKRYTSTKEVLDATLIHQSITGLYETRIDLSQLGGD |
| | (SEQ ID NO: 95) |
| Cas9 nickase | DKKYSIGLDIGTNSVGWAVITDEYKVPSKKFKVLGNTDRHSIKKNLIGALLFDSGET |
| (Met minus) | AEATRLKRTARRRYTRRKNRICYLQEIFSNEMAKVDDSFFHRLEESFLVEEDKKHER |
| Streptococcus | HPIFGNIVDEVAYHEKYPTIYHLRKKLVDSTDKADLRLIYLALAHMIKFRGHFLIEGD |
| pyogenes | LNPDNSDVDKLFIQLVQTYNQLFEENPINASGVDAKAILSARLSKSRRLENLIAQLPG |
| Q99ZW2 Cas9 | EKKNGLFGNLIALSLGLTPNFKSNFDLAEDAKLQLSKDTYDDDLDNLLAQIGDQYA |
| with R863A, | DLFLAAKNLSDAILLSDILRVNTEITKAPLSASMIKRYDEHHQDLTLLKALVRQQLPE |
| wherein X is | KYKEIFFDQSKNGYAGYIDGGASQEEFYKFIKPILEKMDGTEELLVKLNREDLLRKQ |
| any alternate | RTFDNGSIPHQIHLGELHAILRRQEDFYPFLKDNREKIEKILTFRIPYYVGPLARGNSR |
| amino acid | FAWMTRKSEETITPWNFEEVVDKGASAQSFIERMTNFDKNLPNEKVLPKHSLLYEY |
| | FTVYNELTKVKYVTEGMRKPAFLSGEQKKAIVDLLFKTNRKVTVKQLKEDYFKKIE |
| | CFDSVEISGVEDRFNASLGTYHDLLKIIKDKDFLDNEENEDILEDIVLTLTLFEDREMI |
| | EERLKTYAHLFDDKVMKQLKRRRYTGWGRLSRKLINGIRDKQSGKTILDFLKSDGF |
| | ANRNFMQLIHDDSLTFKEDIQKAQVSGQGDSLHEHIANLAGSPAIKKGILQTVKVVD |
| | ELVKVMGRHKPENIVIEMARENQTTQKGQKNSRERMKRIEEGIKELGSQILKEHPVE |
| | NTQLQNEKLYLYYLONGRDMYVDQELDINRLSDYDVDHIVPQSFLKDDSIDNKVLT |
| | RSDKNAGKSDNVPSEEVVKKMKNYWRQLLNAKLITQRKFDNLTKAERGGLSELDK |
| | AGFIKRQLVETRQITKHVAQILDSRMNTKYDENDKLIREVKVITLKSKLVSDFRKDF |
| | QFYKVREINNYHHAHDAYLNAVVGTALIKKYPKLESEFVYGDYKVYDVRKMIAKS |
| | EQEIGKATAKYFFYSNIMNFFKTEITLANGEIRKRPLIETNGETGEIVWDKGRDFATV |
| | RKVLSMPQVNIVKKTEVQTGGFSKESILPKRNSDKLIARKKDWDPKKYGGFDSPTV |
| | AYSVLVVAKVEKGKSKKLKSVKELLGITIMERSSFEKNPIDFLEAKGYKEVKKDLIIK |
| | LPKYSLFELENGRKRMLASAGELQKGNELALPSKYVNFLYLASHYEKLKGSPEDNE |
| | QKQLFVEQHKHYLDEIIEQISEFSKRVILADANLDKVLSAYNKHRDKPIREQAENIIH |
| | LFTLTNLGAPAAFKYFDTTIDRKRYTSTKEVLDATLIHQSITGLYETRIDLSQLGGD |
| | (SEQ ID NO: 96) |

### (5) Other Cas9 Variants

Besides dead Cas9 and Cas9 nickase variants, the Cas9 proteins used herein may also include other “Cas9 variants” having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 protein, including any wild type Cas9, or mutant Cas9 (e.g., a dead Cas9 or Cas9 nickase), or fragment Cas9, or circular permutant Cas9, or other variant of Cas9 disclosed herein or known in the art. In some embodiments, a Cas9 variant may have 1, 2,3, 4,5, 6,7, 8, 9,10,11, 12,13, 14,15, 16,17, 18, 19,20,21, 22, 21,24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a reference Cas9. In some embodiments, the Cas9 variant comprises a fragment of a reference Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SEQ ID NO: 59).

In some embodiments, the disclosure also may utilize Cas9 fragments which retain their functionality and which are fragments of any herein disclosed Cas9 protein. In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length.

In various embodiments, the base editors disclosed herein may comprise one of the Cas9 variants described as follows, or a Cas9 variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 variants.

### (6) Small-Sized Cas9 Variants

In some embodiments, the base editors contemplated herein can include a Cas9 protein that is of smaller molecular weight than the canonical SpCas9 sequence. In some embodiments, the smaller-sized Cas9 variants may facilitate delivery to cells, e.g., by an expression vector, nanoparticle, or other means of delivery.

The canonical SpCas9 protein is 1368 amino acids in length and has a predicted molecular weight of 158 kilodaltons. The term “small-sized Cas9 variant”, as used herein, refers to any Cas9 variant—naturally occurring, engineered, or otherwise—that is less than at least 1300 amino acids, or at least less than 1290 amino acids, or than less than 1280 amino acids, or less than 1270 amino acid, or less than 1260 amino acid, or less than 1250 amino acids, or less than 1240 amino acids, or less than 1230 amino acids, or less than 1220 amino acids, or less than 1210 amino acids, or less than 1200 amino acids, or less than 1190 amino acids, or less than 1180 amino acids, or less than 1170 amino acids, or less than 1160 amino acids, or less than 1150 amino acids, or less than 1140 amino acids, or less than 1130 amino acids, or less than 1120 amino acids, or less than 1110 amino acids, or less than 1100 amino acids, or less than 1050 amino acids, or less than 1000 amino acids, or less than 950 amino acids, or less than 900 amino acids, or less than 850 amino acids, or less than 800 amino acids, or less than 750 amino acids, or less than 700 amino acids, or less than 650 amino acids, or less than 600 amino acids, or less than 550 amino acids, or less than 500 amino acids, but at least larger than about 400 amino acids and retaining the required functions of the Cas9 protein.

In various embodiments, the base editors disclosed herein may comprise one of the small-sized Cas9 variants described as follows, or a Cas9 variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference small-sized Cas9 protein.

| | | SEQ |
|---|---|---|
| | | ID |
| Description | Sequence | NO: |
| SaCas9 | MGKRNYILGLDIGITSVGYGIIDYETRDVIDAGVRLFKEANVENNEGRRSKRG | 67 |
| Staphylococcus | ARRLKRRRRHRIQRVKKLLFDYNLLTDHSELSGINPYEARVKGLSQKLSEEEF | |
| aureus | SAALLHLAKRRGVHNVNEVEEDTGNELSTKEQISRNSKALEEKYVAELQLER | |
| 1053 AA | LKKDGEVRGSINRFKTSDYVKEAKQLLKVQKAYHQLDQSFIDTYIDLLETRRT | |
| 123 kDa | YYEGPGEGSPFGWKDIKEWYEMLMGHCTYFPEELRSVKYAYNADLYNALN | |
| | DLNNLVITRDENEKLEYYEKFQIIENVFKQKKKPTLKQIAKEILVNEEDIKGYR | |
| | VTSTGKPEFTNLKVYHDIKDITARKEIIENAELLDQIAKILTIYQSSEDIQEELTN | |
| | LNSELTQEEIEQISNLKGYTGTHNLSLKAINLILDELWHTNDNQIAIFNRLKLVPK | |
| | KVDLSQQKEIPTTLVDDFILSPVVKRSFIQSIKVINAIIKKYGLPNDIIIELAREK | |
| | NSKDAQKMINEMQKRNRQTNERIEEIIRTTGKENAKYLIEKIKLHDMQEGKCL | |
| | YSLEAIPLEDLLNNPFNYEVDHIIPRSVSFDNSFNNKVLVKQEENSKKGNRTPF | |
| | QYLSSSDSKISYETFKKHILNLAKGKGRISKTKKEYLLEERDINRFSVQKDFINR | |
| | NLVDTRYATRGLMNLLRSYFRVNNLDVKVKSINGGFTSFLRRKWKFKKERN | |
| | KGYKHHAEDALIIANADFIFKEWKKLDKAKKVMENQMFEEKQAESMPEIETE | |
| | QEYKEIFITPHQIKHIKDFKDYKYSHRVDKKPNRKLINDTLYSTRKDDKGNTLI | |
| | VNNLNGLYDKDNDKLKKLINKSPEKLLMYHHDPQTYQKLKLIMEQYGDEKN | |
| | PLYKYYEETGNYLTKYSKKDNGPVIKKIKYYGNKLNAHLDITDDYPNSRNKV | |
| | VKLSLKPYRFDVYLDNGVYKFVTVKNLDVIKKENYYEVNSKCYEEAKKLKK | |
| | ISNQAEFIASFYKNDLIKINGELYRVIGVNNDLLNRIEVNMIDITYREYLENMN | |
| | DKRPPHIIKTIASKTQSIKKYSTDILGNLYEVKSKKHPQIIKK | |
| NmeCas9 | MAAFKPNSINYILGLDIGIASVGWAMVEIDEEENPIRLIDLGVRVFERAEVPKT | 97 |
| N. meningitidis | GDSLAMARRLARSVRRLTRRRAHRLLRTRRLLKREGVLQAANFDENGLIKSL | |
| 1083 AA | PNTPWQLRAAALDRKLTPLEWSAVLLHLIKHRGYLSQRKNEGETADKELGAL | |
| 124.5 kDa | LKGVAGNAHALQTGDFRTPAELALNKFEKESGHIRNQRSDYSHTFSRKDLQA | |
| | ELILLFEKQKEFGNPHVSGGLKEGIETLLMTQRPALSGDAVQKMLGHCTFEPA | |
| | EPKAAKNTYTAERFIWLTKLNNLRILEQGSERPLTDTERATLMDEPYRKSKLT | |
| | YAQARKLLGLEDTAFFKGLRYGKDNAEASTLMEMKAYHAISRALEKEGLKD | |
| | KKSPLNLSPELQDEIGTAFSLFKTDEDITGRLKDRIQPEILEALLKHISFDKFVQI | |
| | SLKALRRIVPLMEQGKRYDEACAEIYGDHYGKKNTEEKIYLPPIPADEIRNPV | |
| | VLRALSQARKVINGVVRRYGSPARIHIETAREVGKSFKDRKEIEKRQEENRKD | |
| | REKAAAKFREYFPNFVGEPKSKDILKLRLYEQQHGKCLYSGKEINLGRLNEK | |
| | GYVEIDAALPFSRTWDDSFNNKVLVLGSENQNKGNQTPYEYFNGKDNSREW | |
| | QEFKARVETSRFPRSKKQRILLQKFDEDGFKERNLNDTRYVNRFLCQFVADR | |
| | MRLTGKGKKRVFASNGQITNLLRGFWGLRKVRAENDRHHALDAVVVACST | |
| | VAMQQKITRFVRYKEMNAFDGKTIDKETGEVLHQKTHFPQPWEFFAQEVMIR | |
| | VFGKPDGKPEFEEADTLEKLRTLLAEKLSSRPEAVHEYVTPLFVSRAPNRKMS | |
| | GQGHMETVKSAKRLDEGVSVLRVPLTQLKLKDLEKMVNREREPKLYEALKA | |
| | RLEAHKDDPAKAFAEPFYKYDKAGNRTQQVKAVRVEQVQKTGVWVRNHN | |
| | GIADNATMVRVDVFEKGDKYYLVPIYSWQVAKGILPDRAVVQGKDEEDWQ | |
| | LIDDSFNFKFSLHPNDLVEVITKKARMFGYFASCHRGTGNINIRIHDLDHKIGK | |
| | NGILEGIGVKTALSFQKYQIDELGKEIRPCRLKKRPPVR | |
| CjCas9 | MARILAFDIGISSIGWAFSENDELKDCGVRIFTKVENPKTGESLALPRRLARSA | 98 |
| C. jejuni | RKRLARRKARLNHLKHLIANEFKLNYEDYQSFDESLAKAYKGSLISPYELRFR | |
| 984 AA | ALNELLSKQDFARVILHIAKRRGYDDIKNSDDKEKGAILKAIKQNEEKLANYQ | |
| 114.9 kDa | SVGEYLYKEYFQKFKENSKEFTNVRNKKESYERCIAQSFLKDELKLIFKKQRE | |
| | FGFSFSKKFEEEVLSVAFYKRALKDFSHLVGNCSFFTDEKRAPKNSPLAFMFV | |
| | ALTRIINLLNNLKNTEGILYTKDDLNALLNEVLKNGTLTYKQTKKLLGLSDDY | |
| | EFKGEKGTYFIEFKKYKEFIKALGEHNLSQDDLNEIAKDITLIKDEIKLKKALA | |
| | KYDLNQNQIDSLSKLEFKDHLNISFKALKLVTPLMLEGKKYDEACNELNLKV | |
| | AINEDKKDFLPAFNETYYKDEVTNPVVLRAIKEYRKVLNALLKKYGKVHKIN | |
| | IELAREVGKNHSQRAKIEKEQNENYKAKKDAELECEKLGLKINSKNILKLRLF | |
| | KEQKEFCAYSGEKIKISDLQDEKMLEIDHIYPYSRSFDDSYMNKVLVFTKQNQ | |
| | EKLNQTPFEAFGNDSAKWQKIEVLAKNLPTKKQKRILDKNYKDKEQKNFKD | |
| | RNLNDTRYIARLVLNYTKDYLDFLPLSDDENTKLNDTQKGSKVHVEAKSGM | |
| | LTSALRHTWGFSAKDRNNHLHHAIDAVIIAYANNSIVKAFSDFKKEQESNSAE | |
| | LYAKKISELDYKNKRKFFEPFSGFRQKVLDKIDEIFVSKPERKKPSGALHEETF | |
| | RKEEEFYQSYGGKEGVLKALELGKIRKVNGKIVKNGDMFRVDIFKHKKTNKF | |
| | YAVPIYTMDFALKVLPNKAVARSKKGEIKDWILMDENYEFCFSLYKDSLILIQ | |
| | TKDMQEPEFVYYNAFTSSTVSLIVSKHDNKFETLSKNQKILFKNANEKEVIAK | |
| | SIGIQNLKVFEKYIVSALGEVTKAEFRQREDFKK | |
| GeoCas9 | MRYKIGLDIGITSVGWAVMNLDIPRIEDLGVRIFDRAENPQTGESLALPRRLAR | 99 |
| G. stearothermophilus | SARRRLRRRKHRLERIRRLVIREGILTKEELDKLFEEKHEIDVWQLRVEALDR | |
| 1087 AA | KLNNDELARVLLHLAKRRGFKSNRKSERSNKENSTMLKHIEENRAILSSYRTV | |
| 127 kDa | GEMIVKDPKFALHKRNKGENYTNTIARDDLEREIRLIFSKQREFGNMSCTEEF | |
| | ENEYITIWASQRPVASKDDIEKKVGFCTFEPKEKRAPKATYTFQSFIAWEHINK | |
| | LRLISPSGARGLTDEERRLLYEQAFQKNKITYHDIRTLLHLPDDTYFKGIVYDR | |
| | GESRKQNENIRFLELDAYHQIRKAVDKVYGKGKSSSFLPIDFDTFGYALTLFK | |
| | DDADIHSYLRNEYEQNGKRMPNLANKVYDNELIEELLNLSFTKFGHLSLKAL | |
| | RSILPYMEQGEVYSSACERAGYTFTGPKKKQKTMLLPNIPPIANPVVMRALTQ | |
| | ARKVVNAIIKKYGSPVSIHIELARDLSQTFDERRKTKKEQDENRKKNETAIRQL | |
| | MEYGLTLNPTGHDIVKFKLWSEQNGRCAYSLQPIEIERLLEPGYVEVDHVIPY | |
| | SRSLDDSYTNKVLVLTRENREKGNRIPAEYLGVGTERWQQFETFVLTNKQFS | |
| | KKKRDRLLRLHYDENEETEFKNRNLNDTRYISRFFANFIREHLKFAESDDKQK | |
| | VYTVNGRVTAHLRSRWEFNKNREESDLHHAVDAVIVACTTPSDIAKVTAFYQ | |
| | RREQNKELAKKTEPHFPQPWPHFADELRARLSKHPKESIKALNLGNYDDQKL | |
| | ESLQPVFVSRMPKRSVTGAAHQETLRRYVGIDERSGKIQTVVKTKLSEIKLDA | |
| | SGHFPMYGKESDPRTYEAIRQRLLEHNNDPKKAFQEPLYKPKKNGEPGPVIRT | |
| | VKIIDTKNQVIPLNDGKTVAYNSNIVRVDVFEKDGKYYCVPVYTMDIMKGILP | |
| | NKAIEPNKPYSEWKEMTEDYTFRFSLYPNDLIRIELPREKTVKTAAGEEINVKD | |
| | VFVYYKTIDSANGGLELISHDHRFSLRGVGSRTLKRFEKYQVDVLGNIYKVRG | |
| | EKRVGLASSAHSKPGKTIRPLQSTRD | |
| LbaCas12a | MSKLEKFTNCYSLSKTLRFKAIPVGKTQENIDNKRLLVEDEKRAEDYKGVKK | 100 |
| L. bacterium | LLDRYYLSFINDVLHSIKLKNLNNYISLFRKKTRTEKENKELENLEINLRKEIA | |
| 1228 AA | KAFKGNEGYKSLFKKDIIETILPEFLDDKDEIALVNSFNGFTTAFTGFFDNREN | |
| 143.9 kDa | MFSEEAKSTSIAFRCINENLTRYISNMDIFEKVDAIFDKHEVQEIKEKILNSDYD | |
| | VEDFFEGEFFNFVLTQEGIDVYNAIIGGFVTESGEKIKGLNEYINLYNQKTKQK | |
| | LPKFKPLYKQVLSDRESLSFYGEGYTSDEEVLEVFRNTLNKNSEIFSSIKKLEK | |
| | LFKNFDEYSSAGIFVKNGPAISTISKDIFGEWNVIRDKWNAEYDDIHLKKKAV | |
| | VTEKYEDDRRKSFKKIGSFSLEQLQEYADADLSVVEKLKEIIIQKVDEIYKVYG | |
| | SSEKLFDADFVLEKSLKKNDAVVAIMKDLLDSVKSFENYIKAFFGEGKETNR | |
| | DESFYGDFVLAYDILLKVDHIYDAIRNYVTQKPYSKDKFKLYFQNPQFMGGW | |
| | DKDKETDYRATILRYGSKYYLAIMDKKYAKCLQKIDKDDVNGNYEKINYKL | |
| | LPGPNKMLPKVFFSKKWMAYYNPSEDIQKIYKNGTFKKGDMFNLNDCHKLI | |
| | DFFKDSISRYPKWSNAYDFNFSETEKYKDIAGFYREVEEQGYKVSFESASKKE | |
| | VDKLVEEGKLYMFQIYNKDFSDKSHGTPNLHTMYFKLLFDENNHGQIRLSGG | |
| | AELFMRRASLKKEELVVHPANSPIANKNPDNPKKTTTLSYDVYKDKRFSEDQ | |
| | YELHIPIAINKCPKNIFKINTEVRVLLKHDDNPYVIGIDRGERNLLYIVVVDGK | |
| | GNIVEQYSLNEIINNFNGIRIKTDYHSLLDKKEKERFEARQNWTSIENIKELKA | |
| | GYISQVVHKICELVEKYDAVIALEDLNSGFKNSRVKVEKQVYQKFEKMLIDK | |
| | LNYMVDKKSNPCATGGALKGYQITNKFESFKSMSTQNGFIFYIPAWLTSKIDP | |
| | STGFVNLLKTKYTSIADSKKFISSFDRIMYVPEEDLFEFALDYKNFSRTDADYI | |
| | KKWKLYSYGNRIRIFRNPKKNNVFDWEEVCLTSAYKELFNKYGINYQQGDIR | |
| | ALLCEQSDKAFYSSFMALMSLMLQMRNSITGRTDVDFLISPVKNSDGIFYDSR | |
| | NYEAQENAILPKNADANGAYNIARKVLWAIGQFKKAEDEKLDKVKIAISNKE | |
| | WLEYAQTSVKH | |
| BhCas12b | MATRSFILKIEPNEEVKKGLWKTHEVLNHGIAYYMNILKLIRQEAIYEHHEQD | 101 |
| B. hisashii | PKNPKKVSKAEIQAELWDFVLKMQKCNSFTHEVDKDEVFNILRELYEELVPSS | |
| 1108 AA | VEKKGEANQLSNKFLYPLVDPNSQSGKGTASSGRKPRWYNLKIAGDPSWEEE | |
| 130.4 kDa | KKKWEEDKKKDPLAKILGKLAEYGLIPLFIPYTDSNEPIVKEIKWMEKSRNQS | |
| | VRRLDKDMFIQALERFLSWESWNLKVKEEYEKVEKEYKTLEERIKEDIQALK | |
| | ALEQYEKERQEQLLRDTLNTNEYRLSKRGLRGWREIIQKWLKMDENEPSEKY | |
| | LEVFKDYQRKHPREAGDYSVYEFLSKKENHFIWRNHPEYPYLYATFCEIDKK | |
| | KKDAKQQATFTLADPINHPLWVRFEERSGSNLNKYRILTEQLHTEKLKKKLT | |
| | VQLDRLIYPTESGGWEEKGKVDIVLLPSRQFYNQIFLDIEEKGKHAFTYKDESI | |
| | KFPLKGTLGGARVQFDRDHLRRYPHKVESGNVGRIYFNMTVNIEPTESPVSKS | |
| | LKIHRDDFPKVVNFKPKELTEWIKDSKGKKLKSGIESLEIGLRVMSIDLGQRQ | |
| | AAAASIFEVVDQKPDIEGKLFFPIKGTELYAVHRASFNIKLPGETLVKSREVLR | |
| | KAREDNLKLMNQKLNFLRNVLHFQQFEDITEREKRVTKWISRQENSDVPLVY | |
| | QDELIQIRELMYKPYKDWVAFLKQLHKRLEVEIGKEVKHWRKSLSDGRKGL | |
| | YGISLKNIDEIDRTRKFLLRWSLRPTEPGEVRRLEPGQRFAIDQLNHLNALKED | |
| | RLKKMANTIIMHALGYCYDVRKKKWQAKNPACQIILFEDLSNYNPYEERSRF | |
| | ENSKLMKWSRREIPRQVALQGEIYGLQVGEVGAQFSSRFHAKTGSPGIRCSVV | |
| | TKEKLQDNRFFKNLQREGRLTLDKIAVLKEGDLYPDKGGEKFISLSKDRKCVT | |
| | THADINAAQNLQKRFWTRTHGFYKVYCKAYQVDGQTVYIPESKDQKQKIIEE | |
| | FGEGYFILKDGVYEWVNAGKLKIKKGSSKQSSSELVDSDILKDSFDLASELKG | |
| | EKLMLYRDPSGNVFPSDKWMAAGVFFGKLERILISKLTNQYSISTIEDDSSKQS | |
| | M | |

### (7) Cas9 Equivalents

In some embodiments, the base editors described herein can include any Cas9 equivalent. As used herein, the term “Cas9 equivalent” is a broad term that encompasses any napDNAbp protein that serves the same function as Cas9 in the present base editors despite that its amino acid primary sequence and/or its three-dimensional structure may be different and/or unrelated from an evolutionary standpoint. Thus, while Cas9 equivalents include any Cas9 ortholog, homolog, mutant, or variant described or embraced herein that are evolutionarily related, the Cas9 equivalents also embrace proteins that may have evolved through convergent evolution processes to have the same or similar function as Cas9, but which do not necessarily have any similarity with regard to amino acid sequence and/or three dimensional structure. The base editors described here embrace any Cas9 equivalent that would provide the same or similar function as Cas9 despite that the Cas9 equivalent may be based on a protein that arose through convergent evolution.

For example, CasX is a Cas9 equivalent that reportedly has the same function as Cas9 but which evolved through convergent evolution. Thus, the CasX protein described in Liu et al., “CasX enzymes comprises a distinct family of RNA-guided genome editors,” Nature, 2019, Vol. 566: 218-223, is contemplated to be used with the base editors described herein. In addition, any variant or modification of CasX is conceivable and within the scope of the present disclosure.

Cas9 is a bacterial enzyme that evolved in a wide variety of species. However, the Cas9 equivalents contemplated herein may also be obtained from archaea, which constitute a domain and kingdom of single-celled prokaryotic microbes different from bacteria.

In some embodiments, Cas9 equivalents may refer to CasX or CasY, which have been described in, for example, Burstein et al., “New CRISPR-Cas systems from uncultivated microbes.” Cell Res. 2017 Feb. 21. doi: 10.1038/cr.2017.21, the entire contents of which is hereby incorporated by reference. Using genome-resolved metagenomics, a number of CRISPR-Cas systems were identified, including the first reported Cas9 in the archaeal domain of life. This divergent Cas9 protein was found in little-studied nanoarchaea as part of an active CRISPR-Cas system. In bacteria, two previously unknown systems were discovered, CRISPR-CasX and CRISPR-CasY, which are among the most compact systems yet discovered. In some embodiments, Cas9 refers to CasX, or a variant of CasX. In some embodiments, Cas9 refers to a CasY, or a variant of CasY. It should be appreciated that other RNA-guided DNA binding proteins may be used as a nucleic acid programmable DNA binding protein (napDNAbp), and are within the scope of this disclosure. Also see Liu et al., “CasX enzymes comprises a distinct family of RNA-guided genome editors,” Nature, 2019, Vol. 566: 218-223. Any of these Cas9 equivalents are contemplated.

In some embodiments, the Cas9 equivalent comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring CasX or CasY protein. In some embodiments, the napDNAbp is a naturally-occurring CasX or CasY protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a wild-type Cas moiety or any Cas moiety provided herein.

In various embodiments, the nucleic acid programmable DNA binding proteins include, without limitation, Cas9 (e.g., dCas9 and nCas9), CasX, CasY, Cpf1, C2c1, C2c2, C2C3, Argonaute, Cas12a, and Cas12b. One example of a nucleic acid programmable DNA-binding protein that has different PAM specificity than Cas9 is Clustered Regularly Interspaced Short Palindromic Repeats from Prevotella and Francisella 1 (Cpf1). Similar to Cas9, Cpf1 is also a class 2 CRISPR effector. It has been shown that Cpf1 mediates robust DNA interference with features distinct from Cas9. Cpf1 is a single RNA-guided endonuclease lacking tracrRNA, and it utilizes a T-rich protospacer-adjacent motif (TTN, TTTN, or YTN). Moreover, Cpf1 cleaves DNA via a staggered DNA double-stranded break. Out of 16 Cpf1-family proteins, two enzymes from Acidaminococcus and Lachnospiraceae are shown to have efficient genome-editing activity in human cells. Cpf1 proteins are known in the art and have been described previously, for example Yamano et al., “Crystal structure of Cpf1 in complex with guide RNA and target DNA.” Cell (165) 2016, p. 949-962; the entire contents of which is hereby incorporated by reference. The state of the art may also now refer to Cpf1 enzymes as Cas12a.

In still other embodiments, the Cas protein may include any CRISPR associated protein, including but not limited to, Cas12a, Cas12b, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof, and preferably comprising a nickase mutation (e.g., a mutation corresponding to the D10A mutation of the wild type Cas9 polypeptide of SEQ ID NO: 59).

In various other embodiments, the napDNAbp can be any of the following proteins: a Cas9, a Cpf1, a CasX, a CasY, a C2c1, a C2c2, a C2c, a GeoCas9, a CjCas9, a Cas12a, a Cas12b, a Cas12g, a Cas12h, a Cas12i, a Cas13b, a Cas13c, a Cas13d, a Cas14, a Csn2, an xCas9, an SpCas9-NG, a circularly permuted Cas9, or an Argonaute (Ago) domain, or a variant thereof.

Exemplary Cas9 equivalent protein sequences can include the following:

| Description | Sequence |
|---|---|
| AsCas12a | MTQFEGFTNLYQVSKTLRFELIPQGKTLKH |
| (previously | IQEQGFIEEDKARNDHYKELKPIIDRIYKT |
| known as | YADQCLQLVQLDWENLSAAIDSYRKEKTEE |
| Cpf1) | TRNALIEEQATYRNAIHDYFIGRTDNLTDA |
| Acidaminococcus | INKRHAEIYKGLFKAELFNGKVLKQLGTVT |
| sp. | TTEHENALLRSFDKFTTYFSGFYENRKNVF |
| (strain | SAEDISTAIPHRIVQDNFPKFKENCHIFTR |
| BV3L6) | LITAVPSLREHFENVKKAIGIFVSTSIEEV |
| UniProtKB | FSFPFYNQLLTQTQIDLYNQLLGGISREAG |
| U2UMQ6 | TEKIKGLNEVLNLAIQKNDETAHIIASLPH |
| | RFIPLFKQILSDRNTLSFILEEFKSDEEVI |
| | QSFCKYKTLLRNENVLETAEALFNELNSID |
| | LTHIFISHKKLETISSALCDHWDTLRNALY |
| | ERRISELTGKITKSAKEKVQRSLKHEDINL |
| | QEIISAAGKELSEAFKQKTSEILSHAHAAL |
| | DQPLPTTLKKQEEKEILKSQLDSLLGLYHL |
| | LDWFAVDESNEVDPEFSARLTGIKLEMEPS |
| | LSFYNKARNYATKKPYSVEKFKLNFQMPTL |
| | ASGWDVNKEKNNGAILFVKNGLYYLGIMPK |
| | QKGRYKALSFEPTEKTSEGFDKMYYDYFPD |
| | AAKMIPKCSTQLKAVTAHFQTHTTPILLSN |
| | NFIEPLEITKEIYDLNNPEKEPKKFQTAYA |
| | KKTGDQKGYREALCKWIDFTRDFLSKYTKT |
| | TSIDLSSLRPSSQYKDLGEYYAELNPLLYH |
| | ISFQRIAEKEIMDAVETGKLYLFQIYNKDF |
| | AKGHHGKPNLHTLYWTGLFSPENLAKTSIK |
| | LNGQAELFYRPKSRMKRMAHRLGEKMLNKK |
| | LKDQKTPIPDTLYQELYDYVNHRLSHDLSD |
| | EARALLPNVITKEVSHEIIKDRRFTSDKFF |
| | FHVPITLNYQAANSPSKFNQRVNAYLKEHP |
| | ETPIIGIDRGERNLIYITVIDSTGKILEQR |
| | SLNTIQQFDYQKKLDNREKERVAARQAWSV |
| | VGTIKDLKQGYLSQVIHEIVDLMIHYQAVV |
| | VLENLNFGFKSKRTGIAEKAVYQQFEKMLI |
| | DKLNCLVLKDYPAEKVGGVLNPYQLTDQFT |
| | SFAKMGTQSGFLFYVPAPYTSKIDPLTGFV |
| | DPFVWKTIKNHESRKHFLEGFDFLHYDVKT |
| | GDFILHFKMNRNLSFQRGLPGFMPAWDIVF |
| | EKNETQFDAKGTPFIAGKRIVPVIENHRFT |
| | GRYRDLYPANELIALLEEKGIVFRDGSNIL |
| | PKLLENDDSHAIDTMVALIRSVLQMRNSNA |
| | ATGEDYINSPVRDLNGVCFDSRFQNPEWPM |
| | DADANGAYHIALKGQLLLNHLKESKDLKLQ |
| | NGISNQDWLAYIQELRN |
| | (SEQ ID NO: 102) |
| AsCas12a | MTQFEGFTNLYQVSKTLRFELIPQGKTLKH |
| nickase | IQEQGFIEEDKARNDHYKELKPIIDRIYKT |
| (e.g., | YADQCLQLVQLDWENLSAAIDSYRKEKTEE |
| R1226A) | TRNALIEEQATYRNAIHDYFIGRTDNLTDA |
| | INKRHAEIYKGLFKAELFNGKVLKQLGTVT |
| | TTEHENALLRSFDKFTTYFSGFYENRKNVF |
| | SAEDISTAIPHRIVQDNFPKFKENCHIFTR |
| | LITAVPSLREHFENVKKAIGIFVSTSIEEV |
| | FSFPFYNQLLTQTQIDLYNQLLGGISREAG |
| | TEKIKGLNEVLNLAIQKNDETAHIIASLPH |
| | RFIPLFKQILSDRNTLSFILEEFKSDEEVI |
| | QSFCKYKTLLRNENVLETAEALFNELNSID |
| | LTHIFISHKKLETISSALCDHWDTLRNALY |
| | ERRISELTGKITKSAKEKVQRSLKHEDINL |
| | QEIISAAGKELSEAFKQKTSEILSHAHAAL |
| | DQPLPTTLKKQEEKEILKSQLDSLLGLYHL |
| | LDWFAVDESNEVDPEFSARLTGIKLEMEPS |
| | LSFYNKARNYATKKPYSVEKFKLNFQMPTL |
| | ASGWDVNKEKNNGAILFVKNGLYYLGIMPK |
| | QKGRYKALSFEPTEKTSEGFDKMYYDYFPD |
| | AAKMIPKCSTQLKAVTAHFQTHTTPILLSN |
| | NFIEPLEITKEIYDLNNPEKEPKKFQTAYA |
| | KKTGDQKGYREALCKWIDFTRDFLSKYTKT |
| | TSIDLSSLRPSSQYKDLGEYYAELNPLLYH |
| | ISFQRIAEKEIMDAVETGKLYLFQIYNKDF |
| | AKGHHGKPNLHTLYWTGLFSPENLAKTSIK |
| | LNGQAELFYRPKSRMKRMAHRLGEKMLNKK |
| | LKDQKTPIPDTLYQELYDYVNHRLSHDLSD |
| | EARALLPNVITKEVSHEIIKDRRFTSDKFF |
| | FHVPITLNYQAANSPSKFNQRVNAYLKEHP |
| | ETPIIGIDRGERNLIYITVIDSTGKILEQR |
| | SLNTIQQFDYQKKLDNREKERVAARQAWSV |
| | VGTIKDLKQGYLSQVIHEIVDLMIHYQAVV |
| | VLENLNFGFKSKRTGIAEKAVYQQFEKMLI |
| | DKLNCLVLKDYPAEKVGGVLNPYQLTDQFT |
| | SFAKMGTQSGFLFYVPAPYTSKIDPLTGFV |
| | DPFVWKTIKNHESRKHFLEGFDFLHYDVKT |
| | GDFILHFKMNRNLSFQRGLPGFMPAWDIVF |
| | EKNETQFDAKGTPFIAGKRIVPVIENHRFT |
| | GRYRDLYPANELIALLEEKGIVFRDGSNIL |
| | PKLLENDDSHAIDTMVALIRSVLQMANSNA |
| | ATGEDYINSPVRDLNGVCFDSRFQNPEWPM |
| | DADANGAYHIALKGQLLLNHLKESKDLKLQ |
| | NGISNQDWLAYIQELRN |
| | (SEQ ID NO: 103) |
| LbCas12a | MNYKTGLEDFIGKESLSKTLRNALIPTEST |
| (previously | KIHMEEMGVIRDDELRAEKQQELKEIMDDY |
| known as | YRTFIEEKLGQIQGIQWNSLFQKMEETMED |
| Cpf1) | ISVRKDLDKIQNEKRKEICCYFTSDKRFKD |
| Lachnospiraceae | LFNAKLITDILPNFIKDNKEYTEEEKAEKE |
| bacterium | QTRVLFQRFATAFTNYFNQRRNNFSEDNIS |
| GAM79 | TAISFRIVNENSEIHLQNMRAFQRIEQQYP |
| Ref Seq. | EEVCGMEEEYKDMLQEWQMKHIYSVDFYDR |
| WP_ | ELTQPGIEYYNGICGKINEHMNQFCQKNRI |
| 119623382.1 | NKNDFRMKKLHKQILCKKSSYYEIPFRFES |
| | DQEVYDALNEFIKTMKKKEIIRRCVHLGQE |
| | CDDYDLGKIYISSNKYEQISNALYGSWDTI |
| | RKCIKEEYMDALPGKGEKKEEKAEAAAKKE |
| | EYRSIADIDKIISLYGSEMDRTISAKKCIT |
| | EICDMAGQISIDPLVCNSDIKLLQNKEKTT |
| | EIKTILDSFLHVYQWGQTFIVSDIIEKDSY |
| | FYSELEDVLEDFEGITTLYNHVRSYVTQKP |
| | YSTVKFKLHFGSPTLANGWSQSKEYDNNAI |
| | LLMRDQKFYLGIFNVRNKPDKQIIKGHEKE |
| | EKGDYKKMIYNLLPGPSKMLPKVFITSRSG |
| | QETYKPSKHILDGYNEKRHIKSSPKFDLGY |
| | CWDLIDYYKECIHKHPDWKNYDFHFSDTKD |
| | YEDISGFYREVEMQGYQIKWTYISADEIQK |
| | LDEKGQIFLFQIYNKDFSVHSTGKDNLHTM |
| | YLKNLFSEENLKDIVLKLNGEAELFFRKAS |
| | IKTPIVHKKGSVLVNRSYTQTVGNKEIRVS |
| | IPEEYYTEIYNYLNHIGKGKLSSEAQRYLD |
| | EGKIKSFTATKDIVKNYRYCCDHYFLHLPI |
| | TINFKAKSDVAVNERTLAYIAKKEDIHIIG |
| | IDRGERNLLYISVVDVHGNIREQRSFNIVN |
| | GYDYQQKLKDREKSRDAARKNWEEIEKIKE |
| | LKEGYLSMVIHYIAQLVVKYNAVVAMEDLN |
| | YGFKTGRFKVERQVYQKFETMLIEKLHYLV |
| | FKDREVCEEGGVLRGYQLTYIPESLKKVGK |
| | QCGFIFYVPAGYTSKIDPTTGFVNLFSFKN |
| | LTNRESRQDFVGKFDEIRYDRDKKMFEFSF |
| | DYNNYIKKGTILASTKWKVYTNGTRLKRIV |
| | VNGKYTSQSMEVELTDAMEKMLQRAGIEYH |
| | DGKDLKGQIVEKGIEAEIIDIFRLTVQMRN |
| | SRSESEDREYDRLISPVLNDKGEFFDTATA |
| | DKTLPQDADANGAYCIALKGLYEVKQIKEN |
| | WKENEQFPRNKLVQDNKTWFDFMQKKRYL |
| | (SEQ ID NO: 104) |
| PcCas12a- | MAKNFEDFKRLYSLSKTLRFEAKPIGATLD |
| previously | NIVKSGLLDEDEHRAASYVKVKKLIDEYHK |
| known at | VFIDRVLDDGCLPLENKGNNNSLAEYYESY |
| Cpf1 | VSRAQDEDAKKKFKEIQQNLRSVIAKKLTE |
| Prevotella | DKAYANLFGNKLIESYKDKEDKKKIIDSDL |
| copri | IQFINTAESTQLDSMSQDEAKELVKEFWGF |
| Ref Seq. | VTYFYGFFDNRKNMYTAEEKSTGIAYRLVN |
| WP_ | ENLPKFIDNIEAFNRAITRPEIQENMGVLY |
| 119227726.1 | SDFSEYLNVESIQEMFQLDYYNMLLTQKQI |
| | DVYNAIIGGKTDDEHDVKIKGINEYINLYN |
| | QQHKDDKLPKLKALFKQILSDRNAISWLPE |
| | EFNSDQEVLNAIKDCYERLAENVLGDKVLK |
| | SLLGSLADYSLDGIFIRNDLQLTDISQKMF |
| | GNWGVIQNAIMQNIKRVAPARKHKESEEDY |
| | EKRIAGIFKKADSFSISYINDCLNEADPNN |
| | AYFVENYFATFGAVNTPTMQRENLFALVQN |
| | AYTEVAALLHSDYPTVKHLAQDKANVSKIK |
| | ALLDAIKSLQHFVKPLLGKGDESDKDERFY |
| | GELASLWAELDTVTPLYNMIRNYMTRKPYS |
| | QKKIKLNFENPQLLGGWDANKEKDYATIIL |
| | RRNGLYYLAIMDKDSRKLLGKAMPSDGECY |
| | EKMVYKFFKDVTTMIPKCSTQLKDVQAYFK |
| | VNTDDYVLNSKAFNKPLTITKEVFDLNNVL |
| | YGKYKKFQKGYLTATGDNVGYTHAVNVWIK |
| | FCMDFLNSYDSTCIYDFSSLKPESYLSLDA |
| | FYQDANLLLYKLSFARASVSYINQLVEEGK |
| | MYLFQIYNKDFSEYSKGTPNMHTLYWKALF |
| | DERNLADVVYKLNGQAEMFYRKKSIENTHP |
| | THPANHPILNKNKDNKKKESLFDYDLIKDR |
| | RYTVDKFMFHVPITMNFKSVGSENINQDVK |
| | AYLRHADDMHIIGIDRGERHLLYLVVIDLQ |
| | GNIKEQYSLNEIVNEYNGNTYHTNYHDLLD |
| | VREEERLKARQSWQTIENIKELKEGYLSQV |
| | IHKITQLMVRYHAIVVLEDLSKGFMRSRQK |
| | VEKQVYQKFEKMLIDKLNYLVDKKTDVSTP |
| | GGLLNAYQLTCKSDSSQKLGKQSGFLFYIP |
| | AWNTSKIDPVTGFVNLLDTHSLNSKEKIKA |
| | FFSKFDAIRYNKDKKWFEFNLDYDKFGKKA |
| | EDTRTKWTLCTRGMRIDTFRNKEKNSQWDN |
| | QEVDLTTEMKSLLEHYYIDIHGNLKDAISA |
| | QTDKAFFTGLLHILKLTLQMRNSITGTETD |
| | YLVSPVADENGIFYDSRSCGNQLPENADAN |
| | GAYNIARKGLMLIEQIKNAEDLNNVKFDIS |
| | NKAWLNFAQQKPYKNG |
| | (SEQ ID NO: 105) |
| ErCas12a- | MFSAKLISDILPEFVIHNNNYSASEKEEKT |
| previously | QVIKLESRFATSFKDYFKNRANCESANDIS |
| known at | SSSCHRIVNDNAEIFFSNALVYRRIVKNLS |
| Cpf1 | NDDINKISGDMKDSLKEMSLEEIYSYEKYG |
| Eubacterium | EFITQEGISFYNDICGKVNLFMNLYCQKNK |
| rectale | ENKNLYKLRKLHKQILCIADTSYEVPYKFE |
| Ref Seq. | SDEEVYQSVNGFLDNISSKHIVERLRKIGE |
| WP_ | NYNGYNLDKIYIVSKFYESVSQKTYRDWET |
| 119223642.1 | INTALEIHYNNILPGNGKSKADKVKKAVKN |
| | DLQKSITEINELVSNYKLCPDDNIKAETYI |
| | HEISHILNNFEAQELKYNPEIHLVESELKA |
| | SELKNVLDVIMNAFHWCSVFMTEELVDKDN |
| | NFYAELEEIYDEIYPVISLYNLVRNYVTQK |
| | PYSTKKIKLNFGIPTLADGWSKSKEYSNNA |
| | IILMRDNLYYLGIFNAKNKPDKKIIEGNTS |
| | ENKGDYKKMIYNLLPGPNKMIPKVFLSSKT |
| | GVETYKPSAYILEGYKQNKHLKSSKDFDIT |
| | FCHDLIDYFKNCIAIHPEWKNFGFDFSDTS |
| | TYEDISGFYREVELQGYKIDWTYISEKDID |
| | LLQEKGQLYLFQIYNKDFSKKSSGNDNLHT |
| | MYLKNLFSEENLKDIVLKLNGEAEIFFRKS |
| | SIKNPIIHKKGSILVNRTYEAEEKDQFGNI |
| | QIVRKTIPENIYQELYKYFNDKSDKELSDE |
| | AAKLKNVVGHHEAATNIVKDYRYTYDKYFL |
| | HMPITINFKANKTSFINDRILQYIAKEKDL |
| | HVIGIDRGERNLIYVSVIDTCGNIVEQKSF |
| | NIVNGYDYQIKLKQQEGARQIARKEWKEIG |
| | KIKEIKEGYLSLVIHEISKMVIKYNAIIAM |
| | EDLSYGFKKGRFKVERQVYQKFETMLINKL |
| | NYLVFKDISITENGGLLKGYQLTYIPDKLK |
| | NVGHQCGCIFYVPAAYTSKIDPTTGFVNIF |
| | KFKDLTVDAKREFIKKFDSIRYDSDKNLFC |
| | FTFDYNNFITQNTVMSKSSWSVYTYGVRIK |
| | RRFVNGRESNESDTIDITKDMEKTLEMTDI |
| | NWRDGHDLRQDIIDYEIVQHIFEIFKLTVQ |
| | MRNSLSELEDRDYDRLISPVLNENNIFYDS |
| | AKAGDALPKDADANGAYCIALKGLYEIKQI |
| | TENWKEDGKFSRDKLKISNKDWFDFIQNKR |
| | YL |
| | (SEQ ID NO: 106) |
| CsCas12a- | PSKMLPKVFITSRSGQETYKPSKHILDGYN |
| previously | EKRHIKSSPKFDLGYCWDLIDYYKECIHKM |
| known at | NYKTGLEDFIGKESLSKTLRNALIPTESTK |
| Cpf1 | IHMEEMGVIRDDELRAEKQQELKEIMDDYY |
| Clostridium | RAFIEEKLGQIQGIQWNSLFQKMEETMEDI |
| sp. AF34- | SVRKDLDKIQNEKRKEICCYFTSDKRFKDL |
| 10BH | FNAKLITDILPNFIKDNKEYTEEEKAEKEQ |
| Ref Seq. | TRVLFQRFATAFTNYFNQRRNNFSEDNIST |
| WP_ | AISFRIVNENSEIHLQNMRAFQRIEQQYPE |
| 118538418.1 | EVCGMEEEYKDMLQEWQMKHIYLVDFYDRV |
| | LTQPGIEYYNGICGKINEHMNQFCQKNRIN |
| | KNDFRMKKLHKQILCKKSSYYEIPFRFESD |
| | QEVYDALNEFIKTMKEKEIICRCVHLGQKC |
| | DDYDLGKIYISSNKYEQISNALYGSWDTIR |
| | KCIKEEYMDALPGKGEKKEEKAEAAAKKEE |
| | YRSIADIDKIISLYGSEMDRTISAKKCITE |
| | ICDMAGQISTDPLVCNSDIKLLQNKEKTTE |
| | IKTILDSFLHVYQWGQTFIVSDIIEKDSYF |
| | YSELEDVLEDFEGITTLYNHVRSYVTQKPY |
| | STVKFKLHFGSPTLANGWSQSKEYDNNAIL |
| | LMRDQKFYLGIFNVRNKPDKQIIKGHEKEE |
| | KGDYKKMIYNLLPGHPDWKNYDFHFSDTKD |
| | YEDISGFYREVEMQGYQIKWTYISADEIQK |
| | LDEKGQIFLFQIYNKDFSVHSTGKDNLHTM |
| | YLKNLFSEENLKDIVLKLNGEAELFFRKAS |
| | IKTPVVHKKGSVLVNRSYTQTVGDKEIRVS |
| | IPEEYYTEIYNYLNHIGRGKLSTEAQRYLE |
| | ERKIKSFTATKDIVKNYRYCCDHYFLHLPI |
| | TINFKAKSDIAVNERTLAYIAKKEDIHIIG |
| | IDRGERNLLYISVVDVHGNIREQRSFNIVN |
| | GYDYQQKLKDREKSRDAARKNWEEIEKIKE |
| | LKEGYLSMVIHYIAQLVVKYNAVVAMEDLN |
| | YGFKTGRFKVERQVYQKFETMLIEKLHYLV |
| | FKDREVCEEGGVLRGYQLTYIPESLKKVGK |
| | QCGFIFYVPAGYTSKIDPTTGFVNLFSFKN |
| | LTNRESRQDFVGKFDEIRYDRDKKMFEFSF |
| | DYNNYIKKGTMLASTKWKVYTNGTRLKRIV |
| | VNGKYTSQSMEVELTDAMEKMLQRAGIEYH |
| | DGKDLKGQIVEKGIEAEIIDIFRLTVQMRN |
| | SRSESEDREYDRLISPVLNDKGEFFDTATA |
| | DKTLPQDADANGAYCIALKGLYEVKQIKEN |
| | WKENEQFPRNKLVQDNKTWFDFMQKKRYL |
| | (SEQ ID NO: 107) |
| BhCas12b | MATRSFILKIEPNEEVKKGLWKTHEVLNHG |
| Bacillus | IAYYMNILKLIRQEAIYEHHEQDPKNPKKV |
| hisashii | SKAEIQAELWDFVLKMQKCNSFTHEVDKDE |
| Ref Seq. | VFNILRELYEELVPSSVEKKGEANQLSNKF |
| WP_ | LYPLVDPNSQSGKGTASSGRKPRWYNLKIA |
| 095142515.1 | GDPSWEEEKKKWEEDKKKDPLAKILGKLAE |
| | YGLIPLFIPYTDSNEPIVKEIKWMEKSRNQ |
| | SVRRLDKDMFIQALERFLSWESWNLKVKEE |
| | YEKVEKEYKTLEERIKEDIQALKALEQYEK |
| | ERQEQLLRDTLNTNEYRLSKRGLRGWREII |
| | QKWLKMDENEPSEKYLEVFKDYQRKHPREA |
| | GDYSVYEFLSKKENHFIWRNHPEYPYLYAT |
| | FCEIDKKKKDAKQQATFTLADPINHPLWVR |
| | FEERSGSNLNKYRILTEQLHTEKLKKKLTV |
| | QLDRLIYPTESGGWEEKGKVDIVLLPSRQF |
| | YNQIFLDIEEKGKHAFTYKDESIKFPLKGT |
| | LGGARVQFDRDHLRRYPHKVESGNVGRIYF |
| | NMTVNIEPTESPVSKSLKIHRDDFPKVVNF |
| | KPKELTEWIKDSKGKKLKSGIESLEIGLRV |
| | MSIDLGQRQAAAASIFEVVDQKPDIEGKLF |
| | FPIKGTELYAVHRASFNIKLPGETLVKSRE |
| | VLRKAREDNLKLMNQKLNFLRNVLHFQQFE |
| | DITEREKRVTKWISRQENSDVPLVYQDELI |
| | QIRELMYKPYKDWVAFLKQLHKRLEVEIGK |
| | EVKHWRKSLSDGRKGLYGISLKNIDEIDRT |
| | RKFLLRWSLRPTEPGEVRRLEPGQRFAIDQ |
| | LNHLNALKEDRLKKMANTIIMHALGYCYDV |
| | RKKKWQAKNPACQIILFEDLSNYNPYEERS |
| | RFENSKLMKWSRREIPRQVALQGEIYGLQV |
| | GEVGAQFSSRFHAKTGSPGIRCSVVTKEKL |
| | QDNRFFKNLQREGRLTLDKIAVLKEGDLYP |
| | DKGGEKFISLSKDRKCVTTHADINAAQNLQ |
| | KRFWTRTHGFYKVYCKAYQVDGQTVYIPES |
| | KDQKQKIIEEFGEGYFILKDGVYEWVNAGK |
| | LKIKKGSSKQSSSELVDSDILKDSFDLASE |
| | LKGEKLMLYRDPSGNVFPSDKWMAAGVFFG |
| | KLERILISKLTNQYSISTIEDDSSKQSM |
| | (SEQ ID NO: 101) |
| ThCas12b | MSEKTTQRAYTLRLNRASGECAVCQNNSCD |
| Thermomonas | CWHDALWATHKAVNRGAKAFGDWLLTLRGG |
| hydrothermalis | LCHTLVEMEVPAKGNNPPQRPTDQERRDRR |
| Ref Seq. | VLLALSWLSVEDEHGAPKEFIVATGRDSAD |
| WP_ | DRAKKVEEKLREILEKRDFQEHEIDAWLQD |
| 072754838 | CGPSLKAHIREDAVWVNRRALFDAAVERIK |
| | TLTWEEAWDFLEPFFGTQYFAGIGDGKDKD |
| | DAEGPARQGEKAKDLVQKAGQWLSARFGIG |
| | TGADFMSMAEAYEKIAKWASQAQNGDNGKA |
| | TIEKLACALRPSEPPTLDTVLKCISGPGHK |
| | SATREYLKTLDKKSTVTQEDLNQLRKLADE |
| | DARNCRKKVGKKGKKPWADEVLKDVENSCE |
| | LTYLQDNSPARHREFSVMLDHAARRVSMAH |
| | SWIKKAEQRRRQFESDAQKLKNLQERAPSA |
| | VEWLDRFCESRSMTTGANTGSGYRIRKRAI |
| | EGWSYVVQAWAEASCDTEDKRIAAARKVQA |
| | DPEIEKFGDIQLFEALAADEAICVWRDQEG |
| | TQNPSILIDYVTGKTAEHNQKRFKVPAYRH |
| | PDELRHPVFCDFGNSRWSIQFAIHKEIRDR |
| | DKGAKQDTRQLQNRHGLKMRLWNGRSMTDV |
| | NLHWSSKRLTADLALDQNPNPNPTEVTRAD |
| | RLGRAASSAFDHVKIKNVFNEKEWNGRLQA |
| | PRAELDRIAKLEEQGKTEQAEKLRKRLRWY |
| | VSFSPCLSPSGPFIVYAGQHNIQPKRSGQY |
| | APHAQANKGRARLAQLILSRLPDLRILSVD |
| | LGHRFAAACAVWETLSSDAFRREIQGLNVL |
| | AGGSGEGDLFLHVEMTGDDGKRRTVVYRRI |
| | GPDQLLDNTPHPAPWARLDRQFLIKLQGED |
| | EGVREASNEELWTVHKLEVEVGRTVPLIDR |
| | MVRSGFGKTEKQKERLKKLRELGWISAMPN |
| | EPSAETDEKEGEIRSISRSVDELMSSALGT |
| | LRLALKRHGNRARIAFAMTADYKPMPGGQK |
| | YYFHEAKEASKNDDETKRRDNQIEFLQDAL |
| | SLWHDLFSSPDWEDNEAKKLWQNHIATLPN |
| | YQTPEEISAELKRVERNKKRKENRDKLRTA |
| | AKALAENDQLRQHLHDTWKERWESDDQQWK |
| | ERLRSLKDWIFPRGKAEDNPSIRHVGGLSI |
| | TRINTISGLYQILKAFKMRPEPDDLRKNIP |
| | QKGDDELENFNRRLLEARDRLREQRVKQLA |
| | SRIIEAALGVGRIKIPKNGKLPKRPRTTVD |
| | TPCHAVVIESLKTYRPDDLRTRRENRQLMQ |
| | WSSAKVRKYLKEGCELYGLHFLEVPANYTS |
| | RQCSRTGLPGIRCDDVPTGDFLKAPWWRRA |
| | INTAREKNGGDAKDRFLVDLYDHLNNLQSK |
| | GEALPATVRVPRQGGNLFIAGAQLDDTNKE |
| | RRAIQADLNAAANIGLRALLDPDWRGRWWY |
| | VPCKDGTSEPALDRIEGSTAFNDVRSLPTG |
| | DNSSRRAPREIENLWRDPSGDSLESGTWSP |
| | TRAYWDTVQSRVIELLRRHAGLPTS |
| | (SEQ ID NO: 108) |
| LsCas12b | MSIRSFKLKLKTKSGVNAEQLRRGLWRTHQ |
| Laceyella | LINDGIAYYMNWLVLLRQEDLFIRNKETNE |
| sacchari | IEKRSKEEIQAVLLERVHKQQQRNQWSGEV |
| WP_1322218 | DEQTLLQALRQLYEEIVPSVIGKSGNASLK |
| 94.1 | ARFFLGPLVDPNNKTTKDVSKSGPTPKWKK |
| | MKDAGDPNWVQEYEKYMAERQTLVRLEEMG |
| | LIPLFPMYTDEVGDIHWLPQASGYTRTWDR |
| | DMFQQAIERLLSWESWNRRVRERRAQFEKK |
| | THDFASRFSESDVQWMNKLREYEAQQEKSL |
| | EENAFAPNEPYALTKKALRGWERVYHSWMR |
| | LDSAASEEAYWQEVATCQTAMRGEFGDPAI |
| | YQFLAQKENHDIWRGYPERVIDFAELNHLQ |
| | RELRRAKEDATFTLPDSVDHPLWVRYEAPG |
| | GTNIHGYDLVQDTKRNLTLILDKFILPDEN |
| | GSWHEVKKVPFSLAKSKQFHRQVWLQEEQK |
| | QKKREVVFYDYSTNLPHLGTLAGAKLQWDR |
| | NFLNKRTQQQIEETGEIGKVFFNISVDVRP |
| | AVEVKNGRLQNGLGKALTVLTHPDGTKIVT |
| | GWKAEQLEKWVGESGRVSSLGLDSLSEGLR |
| | VMSIDLGQRTSATVSVFEITKEAPDNPYKF |
| | FYQLEGTEMFAVHQRSFLLALPGENPPQKI |
| | KQMREIRWKERNRIKQQVDQLSAILRLHKK |
| | VNEDERIQAIDKLLQKVASWQLNEEIATAW |
| | NQALSQLYSKAKENDLQWNQAIKNAHHQLE |
| | PVVGKQISLWRKDLSTGRQGIAGLSLWSIE |
| | ELEATKKLLTRWSKRSREPGVVKRIERFET |
| | FAKQIQHHINQVKENRLKQLANLIVMTALG |
| | YKYDQEQKKWIEVYPACQVVLFENLRSYRF |
| | SFERSRRENKKLMEWSHRSIPKLVQMQGEL |
| | FGLQVADVYAAYSSRYHGRTGAPGIRCHAL |
| | TEADLRNETNIIHELIEAGFIKEEHRPYLQ |
| | QGDLVPWSGGELFATLQKPYDNPRILTLHA |
| | DINAAQNIQKRFWHPSMWFRVNCESVMEGE |
| | IVTYVPKNKTVHKKQGKTFRFVKVEGSDVY |
| | EWAKWSKNRNKNTFSSITERKPPSSMILFR |
| | DPSGTFFKEQEWVEQKTFWGKVQSMIQAYM |
| | KKTIVQRMEE |
| | (SEQ ID NO: 109) |
| DtCas12b | MVLGRKDDTAELRRALWTTHEHVNLAVAEV |
| Dsulfonatronum | ERVLLRCRGRSYWTLDRRGDPVHVPESQVA |
| thiodismutans | EDALAMAREAQRRNGWPVVGEDEEILLALR |
| WP_0313864 | YLYEQIVPSCLLDDLGKPLKGDAQKIGTNY |
| 37 | AGPLFDSDTCRRDEGKDVACCGPFHEVAGK |
| | YLGALPEWATPISKQEFDGKDASHLRFKAT |
| | GGDDAFFRVSIEKANAWYEDPANQDALKNK |
| | AYNKDDWKKEKDKGISSWAVKYIQKQLQLG |
| | QDPRTEVRRKLWLELGLLPLFIPVFDKTMV |
| | GNLWNRLAVRLALAHLLSWESWNHRAVQDQ |
| | ALARAKRDELAALFLGMEDGFAGLREYELR |
| | RNESIKQHAFEPVDRPYVVSGRALRSWTRV |
| | REEWLRHGDTQESRKNICNRLQDRLRGKFG |
| | DPDVFHWLAEDGQEALWKERDCVTSFSLLN |
| | DADG |
| | LLEKRKGYALMTFADARLHPRWAMYEAPGG |
| | SNLRTYQIRKTENGLWADVVLLSPRNESAA |
| | VEEKTFNVRLAPSGQLSNVSFDQIQKGSKM |
| | VGRCRYQSANQQFEGLLGGAEILFDRKRIA |
| | NEQHGATDLASKPGHVWFKLTLDVRPQAPQ |
| | GWLDGKGRPALPPEAKHFKTALSNKSKFAD |
| | QVRPGLRVLSVDLGVRSFAACSVFELVRGG |
| | PDQGTYFPAADGRTVDDPEKLWAKHERSFK |
| | ITLPGENPSRKEEIARRAAMEELRSLNGDI |
| | RRLKAILRLSVLQEDDPRTEHLRLFMEAIV |
| | DDPAKSALNAELFKGFGDDRFRSTPDLWKQ |
| | HCHFFHDKAEKVVAERFSRWRTETRPKSSS |
| | WQDWRERRGYAGGKSYWAVTYLEAVRGLIL |
| | RWNMRGRTYGEVNRQDKKQFGTVASALLHH |
| | INQLKEDRIKTGADMIIQAARGFVPRKNGA |
| | GWVQVHEPCRLILFEDLARYRFRTDRSRRE |
| | NSRLMRWSHREIVNEVGMQGELYGLHVDTT |
| | EAGFSSRYLASSGAPGVRCRHLVEEDFHDG |
| | LPGMHLVGELDWLLPKDKDRTANEARRLLG |
| | GMVRPGMLVPWDGGELFATLNAASQLHVIH |
| | ADINAAQNLQRRFWGRCGEAIRIVCNQLSV |
| | DGSTRYEMAKAPKARLLGALQQLKNGDAPF |
| | HLTSIPNSQKPENSYVMTPTNAGKKYRAGP |
| | GEKSSGEEDELALDIVEQAEELAQGRKTFF |
| | RDPSGVFFAPDRWLPSEIYWSRIRRRIWQV |
| | TLERNSSGRQERAEMDEMPY |
| | (SEQ ID NO: 110) |

The base editors described herein may also comprise Cas12a/Cpf1 (dCpf1) variants that may be used as a guide nucleotide sequence-programmable DNA-binding protein domain. The Cas12a/Cpf1 protein has a RuvC-like endonuclease domain that is similar to the RuvC domain of Cas9 but does not have a HNH endonuclease domain, and the N-terminal of Cpf1 does not have the alfa-helical recognition lobe of Cas9. It was shown in Zetsche et al., Cell, 163, 759-771, 2015 (which is incorporated herein by reference) that, the RuvC-like domain of Cpf1 is responsible for cleaving both DNA strands and inactivation of the RuvC-like domain inactivates Cpf1 nuclease activity.

(8) Cas9 Equivalents with Expanded PAM Sequence

In some embodiments, the napDNAbp is a nucleic acid programmable DNA binding protein that does not require a canonical (NGG) PAM sequence. In some embodiments, the napDNAbp is an argonaute protein. One example of such a nucleic acid programmable DNA binding protein is an Argonaute protein from Natronobacterium gregoryi (NgAgo). NgAgo is a ssDNA-guided endonuclease. NgAgo binds 5′ phosphorylated ssDNA of ˜24 nucleotides (gDNA) to guide it to its target site and will make DNA double-strand breaks at the gDNA site. In contrast to Cas9, the NgAgo-gDNA system does not require a protospacer-adjacent motif (PAM). Using a nuclease inactive NgAgo (dNgAgo) can greatly expand the bases that may be targeted. The characterization and use of NgAgo have been described in Gao et al., Nat Biotechnol., 2016 July; 34(7):768-73. PubMed PMID: 27136078; Swarts et al., Nature. 507(7491) (2014):258-61; and Swarts et al., Nucleic Acids Res. 43(10) (2015):5120-9, each of which is incorporated herein by reference.

In some embodiments, the napDNAbp is a prokaryotic homolog of an Argonaute protein. Prokaryotic homologs of Argonaute proteins are known and have been described, for example, in Makarova K., et al., “Prokaryotic homologs of Argonaute proteins are predicted to function as key components of a novel system of defense against mobile genetic elements”, Biol Direct. 2009 Aug. 25; 4:29. doi: 10.1186/1745-6150-4-29, the entire contents of which is hereby incorporated by reference. In some embodiments, the napDNAbp is a Marinitoga piezophila Argunaute (MpAgo) protein. The CRISPR-associated Marinitoga piezophila Argunaute (MpAgo) protein cleaves single-stranded target sequences using 5′-phosphorylated guides. The 5′ guides are used by all known Argonautes. The crystal structure of an MpAgo-RNA complex shows a guide strand binding site comprising residues that block 5′ phosphate interactions. This data suggests the evolution of an Argonaute subclass with noncanonical specificity for a 5′-hydroxylated guide. See, e.g., Kaya et al., “A bacterial Argonaute with noncanonical guide RNA specificity”, Proc Natl Acad Sci USA. 2016 Apr. 12; 113(15):4057-62, the entire contents of which are hereby incorporated by reference). It should be appreciated that other argonaute proteins may be used, and are within the scope of this disclosure.

In some embodiments, the napDNAbp is a single effector of a microbial CRISPR-Cas system. Single effectors of microbial CRISPR-Cas systems include, without limitation, Cas9, Cpf1, C2c1, C2c2, and C2c3. Typically, microbial CRISPR-Cas systems are divided into Class 1 and Class 2 systems. Class 1 systems have multisubunit effector complexes, while Class 2 systems have a single protein effector. For example, Cas9 and Cpf1 are Class 2 effectors. In addition to Cas9 and Cpf1, three distinct Class 2 CRISPR-Cas systems (C2c1, C2c2, and C2c3) have been described by Shmakov et al., “Discovery and Functional Characterization of Diverse Class 2 CRISPR Cas Systems”, Mol. Cell, 2015 Nov. 5; 60(3): 385-397, the entire contents of which is hereby incorporated by reference. Effectors of two of the systems, C2c1 and C2c3, contain RuvC-like endonuclease domains related to Cpf1. A third system, C2c2 contains an effector with two predicated HEPN RNase domains. Production of mature CRISPR RNA is tracrRNA-independent, unlike production of CRISPR RNA by C2c1. C2c1 depends on both CRISPR RNA and tracrRNA for DNA cleavage. Bacterial C2c2 has been shown to possess a unique RNase activity for CRISPR RNA maturation distinct from its RNA-activated single-stranded RNA degradation activity. These RNase functions are different from each other and from the CRISPR RNA-processing behavior of Cpf1. See, e.g., East-Seletsky, et al., “Two distinct RNase activities of CRISPR-C2c2 enable guide-RNA processing and RNA detection”, Nature, 2016 Oct. 13; 538(7624):270-273, the entire contents of which are hereby incorporated by reference. In vitro biochemical analysis of C2c2 in Leptotrichia shahii has shown that C2c2 is guided by a single CRISPR RNA and can be programed to cleave ssRNA targets carrying complementary protospacers. Catalytic residues in the two conserved HEPN domains mediate cleavage. Mutations in the catalytic residues generate catalytically inactive RNA-binding proteins. See e.g., Abudayyeh et al., “C2c2 is a single-component programmable RNA-guided RNA-targeting CRISPR effector”, Science, 2016 Aug. 5; 353(6299), the entire contents of which are hereby incorporated by reference.

The crystal structure of Alicyclobaccillus acidoterrastris C2c1 (AacC2c1) has been reported in complex with a chimeric single-molecule guide RNA (sgRNA). See e.g., Liu et al., “C2c1-sgRNA Complex Structure Reveals RNA-Guided DNA Cleavage Mechanism”, Mol. Cell, 2017 Jan. 19; 65(2):310-322, the entire contents of which are hereby incorporated by reference. The crystal structure has also been reported in Alicyclobacillus acidoterrestris C2c1 bound to target DNAs as ternary complexes. See e.g., Yang et al., “PAM-dependent Target DNA Recognition and Cleavage by C2C1 CRISPR-Cas endonuclease”, Cell, 2016 Dec. 15; 167(7):1814-1828, the entire contents of which are hereby incorporated by reference. Catalytically competent conformations of AacC2c1, both with target and non-target DNA strands, have been captured independently positioned within a single RuvC catalytic pocket, with C2c1-mediated cleavage resulting in a staggered seven-nucleotide break of target DNA. Structural comparisons between C2c1 ternary complexes and previously identified Cas9 and Cpf1 counterparts demonstrate the diversity of mechanisms used by CRISPR-Cas9 systems.

In some embodiments, the napDNAbp may be a C2c1, a C2c2, or a C2c3 protein. In some embodiments, the napDNAbp is a C2c1 protein. In some embodiments, the napDNAbp is a C2c2 protein. In some embodiments, the napDNAbp is a C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring C2c1, C2c2, or C2c3 protein. In some embodiments, the napDNAbp is a naturally-occurring C2c1, C2c2, or C2c3 protein.

Some aspects of the disclosure provide Cas9 domains that have different PAM specificities. Typically, Cas9 proteins, such as Cas9 from S. pyogenes (spCas9), require a canonical NGG PAM sequence to bind a particular nucleic acid region. This may limit the ability to edit desired bases within a genome. In some embodiments, the base editing fusion proteins provided herein may need to be placed at a precise location, for example where a target base is placed within a 4 base region (e.g., a “editing window”), which is approximately 15 bases upstream of the PAM. See Komor, A. C., et al., “Programmable editing of a target base in genomic DNA without double-stranded DNA cleavage” Nature 533, 420-424 (2016), the entire contents of which are hereby incorporated by reference. Accordingly, in some embodiments, any of the fusion proteins provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

For example, a napDNAbp domain with altered PAM specificity, such as a domain with at least 80%, at least 85%, at least 90%, at least 95%, or at least 99% sequence identity with wild type Francisella novicida Cpf1 (SEQ ID NO: 111) (D917, E1006, and D1255), which has the following amino acid sequence:

(SEQ ID NO: 111)
MSIYQEFVNKYSLSKTLRFELIPQGKTLENIKARGLILDDEKRAK
DYKKAKQIIDKYHQFFIEEILSSVCISEDLLQNYSDVYFKLKKSD
DDNLQKDFKSAKDTIKKQISEYIKDSEKFKNLFNQNLIDAKKGQE
SDLILWLKQSKDNGIELFKANSDITDIDEALEIIKSFKGWTTYFK
GFHENRKNVYSSNDIPTSIIYRIVDDNLPKFLENKAKYESLKDKA
PEAINYEQIKKDLAEELTFDIDYKTSEVNQRVFSLDEVFEIANFN
NYLNQSGITKFNTIIGGKFVNGENTKRKGINEYINLYSQQINDKT
LKKYKMSVLFKQILSDTESKSFVIDKLEDDSDVVTTMQSFYEQIA
AFKTVEEKSIKETLSLLFDDLKAQKLDLSKIYFKNDKSLTDLSQQ
VFDDYSVIGTAVLEYITQQIAPKNLDNPSKKEQELIAKKTEKAKY
LSLETIKLALEEFNKHRDIDKQCRFEEILANFAAIPMIFDEIAQN
KDNLAQISIKYQNQGKKDLLQASAEDDVKAIKDLLDQTNNLLHKL
KIFHISQSEDKANILDKDEHFYLVFEECYFELANIVPLYNKIRNY
ITQKPYSDEKFKLNFENSTLANGWDKNKEPDNTAILFIKDDKYYL
GVMNKKNNKIFDDKAIKENKGEGYKKIVYKLLPGANKMLPKVFFS
AKSIKFYNPSEDILRIRNHSTHTKNGSPQKGYEKFEFNIEDCRKF
IDFYKQSISKHPEWKDFGFRFSDTQRYNSIDEFYREVENQGYKLT
FENISESYIDSVVNQGKLYLFQIYNKDFSAYSKGRPNLHTLYWKA
LFDERNLQDVVYKLNGEAELFYRKQSIPKKITHPAKEAIANKNKD
NPKKESVFEYDLIKDKRFTEDKFFFHCPITINFKSSGANKFNDEI
NLLLKEKANDVHILSIDRGERHLAYYTLVDGKGNIIKQDTFNIIG
NDRMKTNYHDKLAAIEKDRDSARKDWKKINNIKEMKEGYLSQVVH
EIAKLVIEYNAIVVFEDLNFGFKRGRFKVEKQVYQKLEKMLIEKL
NYLVFKDNEFDKTGGVLRAYQLTAPFETFKKMGKQTGIIYYVPAG
FTSKICPVTGFVNQLYPKYESVSKSQEFFSKFDKICYNLDKGYFE
FSFDYKNFGDKAAKGKWTIASFGSRLINFRNSDKNHNWDTREVYP
TKELEKLLKDYSIEYGHGECIKAAICGESDKKFFAKLTSVLNTIL
QMRNSKTGTELDYLISPVADVNGNFFDSRQAPKNMPQDADANGAY
HIGLKGLMLLGRIKNNQEGKKLNLVIKNEEYFEFVQNRNN

An additional napDNAbp domain with altered PAM specificity, such as a domain having at least 80%, at least 85%, at least 90%, at least 95%, or at least 99% sequence identity with wild type Geobacillus thermodenitrificans Cas9 (SEQ ID NO: 77), which has the following amino acid sequence:

(SEQ ID NO: 77)
MKYKIGLDIGITSIGWAVINLDIPRIEDLGVRIFDRAENPKTGES
LALPRRLARSARRRLRRRKHRLERIRRLFVREGILTKEELNKLFE
KKHEIDVWQLRVEALDRKLNNDELARILLHLAKRRGFRSNRKSER
TNKENSTMLKHIEENQSILSSYRTVAEMVVKDPKFSLHKRNKEDN
YTNTVARDDLEREIKLIFAKQREYGNIVCTEAFEHEYISIWASQR
PFASKDDIEKKVGFCTFEPKEKRAPKATYTFQSFTVWEHINKLRL
VSPGGIRALTDDERRLIYKQAFHKNKITFHDVRTLLNLPDDTRFK
GLLYDRNTTLKENEKVRFLELGAYHKIRKAIDSVYGKGAAKSFRP
IDFDTFGYALTMFKDDTDIRSYLRNEYEQNGKRMENLADKVYDEE
LIEELLNLSFSKFGHLSLKALRNILPYMEQGEVYSTACERAGYTF
TGPKKKQKTVLLPNIPPIANPVVMRALTQARKVVNAIIKKYGSPV
SIHIELARELSQSFDERRKMQKEQEGNRKKNETAIRQLVEYGLTL
NPTGLDIVKFKLWSEQNGKCAYSLQPIEIERLLEPGYTEVDHVIP
YSRSLDDSYTNKVLVLTKENREKGNRTPAEYLGLGSERWQQFETF
VLTNKQFSKKKRDRLLRLHYDENEENEFKNRNLNDTRYISRFLAN
FIREHLKFADSDDKQKVYTVNGRITAHLRSRWNFNKNREESNLHH
AVDAAIVACTTPSDIARVTAFYQRREQNKELSKKTDPQFPQPWPH
FADELQARLSKNPKESIKALNLGNYDNEKLESLQPVFVSRMPKRS
ITGAAHQETLRRYIGIDERSGKIQTVVKKKLSEIQLDKTGHFPMY
GKESDPRTYEAIRQRLLEHNNDPKKAFQEPLYKPKKNGELGPIIR
TIKIIDTTNQVIPLNDGKTVAYNSNIVRVDVFEKDGKYYCVPIYT
IDMMKGILPNKAIEPNKPYSEWKEMTEDYTFRFSLYPNDLIRIEF
PREKTIKTAVGEEIKIKDLFAYYQTIDSSNGGLSLVSHDNNFSLR
SIGSRTLKRFEKYQVDVLGNIYKVRGEKRVGVASSSHSKAGETIR
PL

In some embodiments, the nucleic acid programmable DNA binding protein (napDNAbp) is a nucleic acid programmable DNA binding protein that does not require a canonical (NGG) PAM sequence. In some embodiments, the napDNAbp is an argonaute protein. One example of such a nucleic acid programmable DNA binding protein is an Argonaute protein from Natronobacterium gregoryi (NgAgo). NgAgo is a ssDNA-guided endonuclease. NgAgo binds 5′ phosphorylated ssDNA of ˜24 nucleotides (gDNA) to guide it to its target site and will make DNA double-strand breaks at the gDNA site. In contrast to Cas9, the NgAgo-gDNA system does not require a protospacer-adjacent motif (PAM). Using a nuclease inactive NgAgo (dNgAgo) can greatly expand the bases that may be targeted. The characterization and use of NgAgo have been described in Gao et al., Nat Biotechnol., 34(7): 768-73 (2016), PubMed PMID: 27136078; Swarts et al., Nature, 507(7491): 258-61 (2014); and Swarts et al., Nucleic Acids Res. 43(10) (2015): 5120-9, each of which is incorporated herein by reference. The sequence of Natronobacterium gregoryi Argonaute is provided in SEQ ID NO: 112.

The disclosed fusion proteins may comprise a napDNAbp domain having at least 80%, at least 85%, at least 90%, at least 95%, or at least 99% sequence identity with wild type Natronobacterium gregoryi Argonaute (SEQ ID NO: 112), which has the following amino acid sequence:

(SEQ ID NO: 112)
MTVIDLDSTTTADELTSGHTYDISVTLTGVYDNTDEQHPRMSLAF
EQDNGERRYITLWKNTTPKDVFTYDYATGSTYIFTNIDYEVKDGY
ENLTATYQTTVENATAQEVGTTDEDETFAGGEPLDHHLDDALNET
PDDAETESDSGHVMTSFASRDQLPEWTLHTYTLTATDGAKTDTEY
ARRTLAYTVRQELYTDHDAAPVATDGLMLLTPEPLGETPLDLDCG
VRVEADETRTLDYTTAKDRLLARELVEEGLKRSLWDDYLVRGIDE
VLSKEPVLTCDEFDLHERYDLSVEVGHSGRAYLHINFRHRFVPKL
TLADIDDDNIYPGLRVKTTYRPRRGHIVWGLRDECATDSLNTLGN
QSVVAYHRNNQTPINTDLLDAIEAADRRVVETRRQGHGDDAVSFP
QELLAVEPNTHQIKQFASDGFHQQARSKTRLSASRCSEKAQAFAE
RLDPVRLNGSTVEFSSEFFTGNNEQQLRLLYENGESVLTFRDGAR
GAHPDETFSKGIVNPPESFEVAVVLPEQQADTCKAQWDTMADLLN
QAGAPPTRSETVQYDAFSSPESISLNVAGAIDPSEVDAAFVVLPP
DQEGFADLASPTETYDELKKALANMGIYSQMAYFDRFRDAKIFYT
RNVALGLLAAAGGVAFTTEHAMPGDADMFIGIDVSRSYPEDGASG
QINIAATATAVYKDGTILGHSSTRPQLGEKLQSTDVRDIMKNAIL
GYQQVTGESPTHIVIHRDGFMNEDLDPATEFLNEQGVEYDIVEIR
KQPQTRLLAVSDVQYDTPVKSIAAINQNEPRATVATFGAPEYLAT
RDGGGLPRPIQIERVAGETDIETLTRQVYLLSQSHIQVHNSTARL
PITTAYADQASTHATKGYLVQTGAFESNVGFL

### (9) Cas9 Circular Permutants

In various embodiments, the base editors disclosed herein may comprise a circular permutant of Cas9.

The term “circularly permuted Cas9” or “circular permutant” of Cas9 or “CP-Cas9”) refers to any Cas9 protein, or variant thereof, that occurs or has been modify to engineered as a circular permutant variant, which means the N-terminus and the C-terminus of a Cas9 protein (e.g., a wild type Cas9 protein) have been topically rearranged. Such circularly permuted Cas9 proteins, or variants thereof, retain the ability to bind DNA when complexed with a guide RNA (gRNA). See, Oakes et al., “Protein Engineering of Cas9 for enhanced function,” Methods Enzymol, 2014, 546: 491-511 and Oakes et al., “CRISPR-Cas9 Circular Permutants as Programmable Scaffolds for Genome Modification,” Cell, Jan. 10, 2019, 176: 254-267, each of are incorporated herein by reference. The instant disclosure contemplates any previously known CP-Cas9 or use a new CP-Cas9 so long as the resulting circularly permuted protein retains the ability to bind DNA when complexed with a guide RNA (gRNA).

Any of the Cas9 proteins described herein, including any variant, ortholog, or naturally occurring Cas9 or equivalent thereof, may be reconfigured as a circular permutant variant.

In various embodiments, the circular permutants of Cas9 may have the following structure:

N-terminus-[original C-terminus]-[optional linker]-[original N-terminus]-C-terminus.

As an example, the present disclosure contemplates the following circular permutants of canonical S. pyogenes Cas9 (1368 amino acids of UniProtKB—Q99ZW2 (CAS9_STRP1) (numbering is based on the amino acid position in SEQ ID NO: 59)):
N-terminus-[1268-1368]-[optional linker]-[1-1267]-C-terminus;
N-terminus-[1168-1368]-[optional linker]-[1-1167]-C-terminus;
N-terminus-[1068-1368]-[optional linker]-[1-1067]-C-terminus;
N-terminus-[968-1368]-[optional linker]-[1-967]-C-terminus;
N-terminus-[868-1368]-[optional linker]-[1-867]-C-terminus;
N-terminus-[768-1368]-[optional linker]-[1-767]-C-terminus;
N-terminus-[668-1368]-[optional linker]-[1-667]-C-terminus;
N-terminus-[568-1368]-[optional linker]-[1-567]-C-terminus;
N-terminus-[468-1368]-[optional linker]-[1-467]-C-terminus;
N-terminus-[368-1368]-[optional linker]-[1-367]-C-terminus;
N-terminus-[268-1368]-[optional linker]-[1-267]-C-terminus;
N-terminus-[168-1368]-[optional linker]-[1-167]-C-terminus;
N-terminus-[68-1368]-[optional linker]-[1-67]-C-terminus; or
N-terminus-[10-1368]-[optional linker]-[1-9]-C-terminus, or the corresponding circular permutants of other Cas9 proteins (including other Cas9 orthologs, variants, etc).

In particular embodiments, the circular permuant Cas9 has the following structure (based on S. pyogenes Cas9 (1368 amino acids of UniProtKB—Q99ZW2 (CAS9_STRP1) (numbering is based on the amino acid position in SEQ ID NO: 59):
N-terminus-[102-1368]-[optional linker]-[1-101]-C-terminus;
N-terminus-[1028-1368]-[optional linker]-[1-1027]-C-terminus;
N-terminus-[1041-1368]-[optional linker]-[1-1043]-C-terminus;
N-terminus-[1249-1368]-[optional linker]-[1-1248]-C-terminus; or
N-terminus-[1300-1368]-[optional linker]-[1-1299]-C-terminus, or the corresponding circular permutants of other Cas9 proteins (including other Cas9 orthologs, variants, etc).

In still other embodiments, the circular permuant Cas9 has the following structure (based on S. pyogenes Cas9 (1368 amino acids of UniProtKB—Q99ZW2 (CAS9_STRP1) (numbering is based on the amino acid position in SEQ ID NO: 59):
N-terminus-[103-1368]-[optional linker]-[1-102]-C-terminus;
N-terminus-[1029-1368]-[optional linker]-[1-1028]-C-terminus;
N-terminus-[1042-1368]-[optional linker]-[1-1041]-C-terminus;
N-terminus-[1250-1368]-[optional linker]-[1-1249]-C-terminus; or
N-terminus-[1301-1368]-[optional linker]-[1-1300]-C-terminus, or the corresponding circular permutants of other Cas9 proteins (including other Cas9 orthologs, variants, etc).

In some embodiments, the circular permutant can be formed by linking a C-terminal fragment of a Cas9 to an N-terminal fragment of a Cas9, either directly or by using a linker, such as an amino acid linker. In some embodiments, The C-terminal fragment may correspond to the C-terminal 95% or more of the amino acids of a Cas9 (e.g., amino acids about 1300-1368), or the C-terminal 90%, 85%, 80%, 75%, 70%, 65%, 60%, 55%, 50%, 45%, 40%, 35%, 30%, 25%, 20%, 15%, 10%, or 5% or more of a Cas9 (e.g., any one of SEQ ID NOs: 59-99). The N-terminal portion may correspond to the N-terminal 95% or more of the amino acids of a Cas9 (e.g., amino acids about 1-1300), or the N-terminal 90%, 85%, 80%, 75%, 70%, 65%, 60%, 55%, 50%, 45%, 40%, 35%, 30%, 25%, 20%, 15%, 10%, or 5% or more of a Cas9 (e.g., of SEQ ID NO: 59-99).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment that is rearranged to the N-terminus, includes or corresponds to the C-terminal 30% or less of the amino acids of a Cas9 (e.g., amino acids 1012-1368 of SEQ ID NO: 59). In some embodiments, the C-terminal fragment that is rearranged to the N-terminus, includes or corresponds to the C-terminal 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% of the amino acids of a Cas9 (e.g., the Cas9 of SEQ ID NO: 59). In some embodiments, the C-terminal fragment that is rearranged to the N-terminus, includes or corresponds to the C-terminal 410 residues or less of a Cas9 (e.g., the Cas9 of SEQ ID NO: 59). In some embodiments, the C-terminal portion that is rearranged to the N-terminus, includes or corresponds to the C-terminal 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, or 10 residues of a Cas9 (e.g., the Cas9 of SEQ ID NO: 59). In some embodiments, the C-terminal portion that is rearranged to the N-terminus, includes or corresponds to the C-terminal 357, 341, 328, 120, or 69 residues of a Cas9 (e.g., the Cas9 of SEQ ID NO: 59).

In other embodiments, circular permutant Cas9 variants may be defined as a topological rearrangement of a Cas9 primary structure based on the following method, which is based on S. pyogenes Cas9 of SEQ ID NO: 59: (a) selecting a circular permutant (CP) site corresponding to an internal amino acid residue of the Cas9 primary structure, which dissects the original protein into two halves: an N-terminal region and a C-terminal region; (b) modifying the Cas9 protein sequence (e.g., by genetic engineering techniques) by moving the original C-terminal region (comprising the CP site amino acid) to precede the original N-terminal region, thereby forming a new N-terminus of the Cas9 protein that now begins with the CP site amino acid residue. The CP site can be located in any domain of the Cas9 protein, including, for example, the helical-II domain, the RuvCIII domain, or the CTD domain. For example, the CP site may be located (relative the S. pyogenes Cas9 of SEQ ID NO: 59) at original amino acid residue 181, 199, 230, 270, 310, 1010, 1016, 1023, 1029, 1041, 1247, 1249, or 1282. Thus, once relocated to the N-terminus, original amino acid 181, 199, 230, 270, 310, 1010, 1016, 1023, 1029, 1041, 1247, 1249, or 1282 would become the new N-terminal amino acid. Nomenclature of these CP-Cas9 proteins may be referred to as Cas9-CP¹⁸¹, Cas9-CP¹⁹⁹, Cas9-CP²³⁰, Cas9-CP²⁷⁰, Cas9-CP³¹⁰, Cas9-CP¹⁰¹⁰, Cas9-CP¹⁰¹⁶, Cas9-CP¹⁰²³, Cas9-CP¹⁰²⁹, Cas9-CP¹⁰⁴¹, Cas9-CP¹²⁴⁷, Cas9-CP¹²⁴⁹, and Cas9-CP¹²⁸², respectively. This description is not meant to be limited to making CP variants from SEQ ID NO: 59, but may be implemented to make CP variants in any Cas9 sequence, either at CP sites that correspond to these positions, or at other CP sites entirely. This description is not meant to limit the specific CP sites in any way. Virtually any CP site may be used to form a CP-Cas9 variant.

Exemplary CP-Cas9 amino acid sequences, based on the Cas9 of SEQ ID NO: 59, are provided below in which linker sequences are indicated by underlining and optional methionine (M) residues are indicated in bold. It should be appreciated that the disclosure provides CP-Cas9 sequences that do not include a linker sequence or that include different linker sequences. It should be appreciated that CP-Cas9 sequences may be based on Cas9 sequences other than that of SEQ ID NO: 59 and any examples provided herein are not meant to be limiting. Exemplary CP-Cas9 sequences are as follows:

| | | SEQ |
|---|---|---|
| CP | | ID |
| name | Sequence | NO: |
| CP1012 | DYKVYDVRKMIAKSEQEIGKATAKYFFYSN | 113 |
| | IMNFFKTEITLANGEIRKRPLIETNGETGE | |
| | IVWDKGRDFATVRKVLSMPQVNIVKKTEVQ | |
| | TGGFSKESILPKRNSDKLIARKKDWDPKKY | |
| | GGFDSPTVAYSVLVVAKVEKGKSKKLKSVK | |
| | ELLGITIMERSSFEKNPIDFLEAKGYKEVK | |
| | KDLIIKLPKYSLFELENGRKRMLASAGELQ | |
| | KGNELALPSKYVNFLYLASHYEKLKGSPED | |
| | NEQKQLFVEQHKHYLDEIIEQISEFSKRVI | |
| | LADANLDKVLSAYNKHRDKPIREQAENIIH | |
| | LFTLTNLGAPAAFKYFDTTIDRKRYTSTKE | |
| | VLDATLIHQSITGLYETRIDLSQLGGDGGS | |
| | GGSGGSGGSGGSGGSGGDKKYSIGLAIGTN | |
| | SVGWAVITDEYKVPSKKFKVLGNTDRHSIK | |
| | KNLIGALLFDSGETAEATRLKRTARRRYTR | |
| | RKNRICYLQEIFSNEMAKVDDSFFHRLEES | |
| | FLVEEDKKHERHPIFGNIVDEVAYHEKYPT | |
| | IYHLRKKLVDSTDKADLRLIYLALAHMIKF | |
| | RGHFLIEGDLNPDNSDVDKLFIQLVQTYNQ | |
| | LFEENPINASGVDAKAILSARLSKSRRLEN | |
| | LIAQLPGEKKNGLFGNLIALSLGLTPNFKS | |
| | NFDLAEDAKLQLSKDTYDDDLDNLLAQIGD | |
| | QYADLFLAAKNLSDAILLSDILRVNTEITK | |
| | APLSASMIKRYDEHHQDLTLLKALVRQQLP | |
| | EKYKEIFFDQSKNGYAGYIDGGASQEEFYK | |
| | FIKPILEKMDGTEELLVKLNREDLLRKQRT | |
| | FDNGSIPHQIHLGELHAILRRQEDFYPFLK | |
| | DNREKIEKILTFRIPYYVGPLARGNSRFAW | |
| | MTRKSEETITPWNFEEVVDKGASAQSFIER | |
| | MTNFDKNLPNEKVLPKHSLLYEYFTVYNEL | |
| | TKVKYVTEGMRKPAFLSGEQKKAIVDLLFK | |
| | TNRKVTVKQLKEDYFKKIECFDSVEISGVE | |
| | DRFNASLGTYHDLLKIIKDKDFLDNEENED | |
| | ILEDIVLTLTLFEDREMIEERLKTYAHLFD | |
| | DKVMKQLKRRRYTGWGRLSRKLINGIRDKQ | |
| | SGKTILDFLKSDGFANRNFMQLIHDDSLTF | |
| | KEDIQKAQVSGQGDSLHEHIANLAGSPAIK | |
| | KGILQTVKVVDELVKVMGRHKPENIVIEMA | |
| | RENQTTQKGQKNSRERMKRIEEGIKELGSQ | |
| | ILKEHPVENTQLQNEKLYLYYLQNGRDMYV | |
| | DQELDINRLSDYDVDHIVPQSFLKDDSIDN | |
| | KVLTRSDKNRGKSDNVPSEEVVKKMKNYWR | |
| | QLLNAKLITQRKFDNLTKAERGGLSELDKA | |
| | GFIKRQLVETRQITKHVAQILDSRMNTKYD | |
| | ENDKLIREVKVITLKSKLVSDFRKDFQFYK | |
| | VREINNYHHAHDAYLNAVVGTALIKKYPKL | |
| | ESEFVYG | |
| CP1028 | EIGKATAKYFFYSNIMNFFKTEITLANGEI | 114 |
| | RKRPLIETNGETGEIVWDKGRDFATVRKVL | |
| | SMPQVNIVKKTEVQTGGFSKESILPKRNSD | |
| | KLIARKKDWDPKKYGGFDSPTVAYSVLVVA | |
| | KVEKGKSKKLKSVKELLGITIMERSSFEKN | |
| | PIDFLEAKGYKEVKKDLIIKLPKYSLFELE | |
| | NGRKRMLASAGELQKGNELALPSKYVNFLY | |
| | LASHYEKLKGSPEDNEQKQLFVEQHKHYLD | |
| | EIIEQISEFSKRVILADANLDKVLSAYNKH | |
| | RDKPIREQAENIIHLFTLTNLGAPAAFKYF | |
| | DTTIDRKRYTSTKEVLDATLIHQSITGLYE | |
| | TRIDLSQLGGDGGSGGSGGSGGSGGSGGSG | |
| | GMDKKYSIGLAIGTNSVGWAVITDEYKVPS | |
| | KKFKVLGNTDRHSIKKNLIGALLFDSGETA | |
| | EATRLKRTARRRYTRRKNRICYLQEIFSNE | |
| | MAKVDDSFFHRLEESFLVEEDKKHERHPIF | |
| | GNIVDEVAYHEKYPTIYHLRKKLVDSTDKA | |
| | DLRLIYLALAHMIKFRGHFLIEGDLNPDNS | |
| | DVDKLFIQLVQTYNQLFEENPINASGVDAK | |
| | AILSARLSKSRRLENLIAQLPGEKKNGLFG | |
| | NLIALSLGLTPNFKSNFDLAEDAKLQLSKD | |
| | TYDDDLDNLLAQIGDQYADLFLAAKNLSDA | |
| | ILLSDILRVNTEITKAPLSASMIKRYDEHH | |
| | QDLTLLKALVRQQLPEKYKEIFFDQSKNGY | |
| | AGYIDGGASQEEFYKFIKPILEKMDGTEEL | |
| | LVKLNREDLLRKQRTFDNGSIPHQIHLGEL | |
| | HAILRRQEDFYPFLKDNREKIEKILTFRIP | |
| | YYVGPLARGNSRFAWMTRKSEETITPWNFE | |
| | EVVDKGASAQSFIERMTNFDKNLPNEKVLP | |
| | KHSLLYEYFTVYNELTKVKYVTEGMRKPAF | |
| | LSGEQKKAIVDLLFKTNRKVTVKQLKEDYF | |
| | KKIECFDSVEISGVEDRFNASLGTYHDLLK | |
| | IIKDKDFLDNEENEDILEDIVLTLTLFEDR | |
| | EMIEERLKTYAHLFDDKVMKQLKRRRYTGW | |
| | GRLSRKLINGIRDKQSGKTILDFLKSDGFA | |
| | NRNFMQLIHDDSLTFKEDIQKAQVSGQGDS | |
| | LHEHIANLAGSPAIKKGILQTVKVVDELVK | |
| | VMGRHKPENIVIEMARENQTTQKGQKNSRE | |
| | RMKRIEEGIKELGSQILKEHPVENTQLQNE | |
| | KLYLYYLQNGRDMYVDQELDINRLSDYDVD | |
| | HIVPQSFLKDDSIDNKVLTRSDKNRGKSDN | |
| | VPSEEVVKKMKNYWRQLLNAKLITQRKFDN | |
| | LTKAERGGLSELDKAGFIKRQLVETRQITK | |
| | HVAQILDSRMNTKYDENDKLIREVKVITLK | |
| | SKLVSDFRKDFQFYKVREINNYHHAHDAYL | |
| | NAVVGTALIKKYPKLESEFVYGDYKVYDVR | |
| | KMIAKSEQ | |
| CP1041 | NIMNFFKTEITLANGEIRKRPLIETNGETG | 115 |
| | EIVWDKGRDFATVRKVLSMPQVNIVKKTEV | |
| | QTGGFSKESILPKRNSDKLIARKKDWDPKK | |
| | YGGFDSPTVAYSVLVVAKVEKGKSKKLKSV | |
| | KELLGITIMERSSFEKNPIDFLEAKGYKEV | |
| | KKDLIIKLPKYSLFELENGRKRMLASAGEL | |
| | QKGNELALPSKYVNFLYLASHYEKLKGSPE | |
| | DNEQKQLFVEQHKHYLDEIIEQISEFSKRV | |
| | ILADANLDKVLSAYNKHRDKPIREQAENII | |
| | HLFTLTNLGAPAAFKYFDTTIDRKRYTSTK | |
| | EVLDATLIHQSITGLYETRIDLSQLGGDGG | |
| | SGGSGGSGGSGGSGGSGGDKKYSIGLAIGT | |
| | NSVGWAVITDEYKVPSKKFKVLGNTDRHSI | |
| | KKNLIGALLFDSGETAEATRLKRTARRRYT | |
| | RRKNRICYLQEIFSNEMAKVDDSFFHRLEE | |
| | SFLVEEDKKHERHPIFGNIVDEVAYHEKYP | |
| | TIYHLRKKLVDSTDKADLRLIYLALAHMIK | |
| | FRGHFLIEGDLNPDNSDVDKLFIQLVQTYN | |
| | QLFEENPINASGVDAKAILSARLSKSRRLE | |
| | NLIAQLPGEKKNGLFGNLIALSLGLTPNFK | |
| | SNFDLAEDAKLQLSKDTYDDDLDNLLAQIG | |
| | DQYADLFLAAKNLSDAILLSDILRVNTEIT | |
| | KAPLSASMIKRYDEHHQDLTLLKALVRQQL | |
| | PEKYKEIFFDQSKNGYAGYIDGGASQEEFY | |
| | KFIKPILEKMDGTEELLVKLNREDLLRKQR | |
| | TFDNGSIPHQIHLGELHAILRRQEDFYPFL | |
| | KDNREKIEKILTFRIPYYVGPLARGNSRFA | |
| | WMTRKSEETITPWNFEEVVDKGASAQSFIE | |
| | RMTNFDKNLPNEKVLPKHSLLYEYFTVYNE | |
| | LTKVKYVTEGMRKPAFLSGEQKKAIVDLLF | |
| | KTNRKVTVKQLKEDYFKKIECFDSVEISGV | |
| | EDRFNASLGTYHDLLKIIKDKDFLDNEENE | |
| | DILEDIVLTLTLFEDREMIEERLKTYAHLF | |
| | DDKVMKQLKRRRYTGWGRLSRKLINGIRDK | |
| | QSGKTILDFLKSDGFANRNFMQLIHDDSLT | |
| | FKEDIQKAQVSGQGDSLHEHIANLAGSPAI | |
| | KKGILQTVKVVDELVKVMGRHKPENIVIEM | |
| | ARENQTTQKGQKNSRERMKRIEEGIKELGS | |
| | QILKEHPVENTQLQNEKLYLYYLQNGRDMY | |
| | VDQELDINRLSDYDVDHIVPQSFLKDDSID | |
| | NKVLTRSDKNRGKSDNVPSEEVVKKMKNYW | |
| | RQLLNAKLITQRKFDNLTKAERGGLSELDK | |
| | AGFIKRQLVETRQITKHVAQILDSRMNTKY | |
| | DENDKLIREVKVITLKSKLVSDFRKDFQFY | |
| | KVREINNYHHAHDAYLNAVVGTALIKKYPK | |
| | LESEFVYGDYKVYDVRKMIAKSEQEIGKAT | |
| | AKYFFYS | |
| CP1249 | PEDNEQKQLFVEQHKHYLDEIIEQISEFSK | 116 |
| | RVILADANLDKVLSAYNKHRDKPIREQAEN | |
| | IIHLFTLTNLGAPAAFKYFDTTIDRKRYTS | |
| | TKEVLDATLIHQSITGLYETRIDLSQLGGD | |
| | GGSGGSGGSGGSGGSGGSGGMDKKYSIGLA | |
| | IGTNSVGWAVITDEYKVPSKKFKVLGNTDR | |
| | HSIKKNLIGALLFDSGETAEATRLKRTARR | |
| | RYTRRKNRICYLQEIFSNEMAKVDDSFFHR | |
| | LEESFLVEEDKKHERHPIFGNIVDEVAYHE | |
| | KYPTIYHLRKKLVDSTDKADLRLIYLALAH | |
| | MIKFRGHFLIEGDLNPDNSDVDKLFIQLVQ | |
| | TYNQLFEENPINASGVDAKAILSARLSKSR | |
| | RLENLIAQLPGEKKNGLFGNLIALSLGLTP | |
| | NFKSNFDLAEDAKLQLSKDTYDDDLDNLLA | |
| | QIGDQYADLFLAAKNLSDAILLSDILRVNT | |
| | EITKAPLSASMIKRYDEHHQDLTLLKALVR | |
| | QQLPEKYKEIFFDQSKNGYAGYIDGGASQE | |
| | EFYKFIKPILEKMDGTEELLVKLNREDLLR | |
| | KQRTFDNGSIPHQIHLGELHAILRRQEDFY | |
| | PFLKDNREKIEKILTFRIPYYVGPLARGNS | |
| | RFAWMTRKSEETITPWNFEEVVDKGASAQS | |
| | FIERMTNFDKNLPNEKVLPKHSLLYEYFTV | |
| | YNELTKVKYVTEGMRKPAFLSGEQKKAIVD | |
| | LLFKTNRKVTVKQLKEDYFKKIECFDSVEI | |
| | SGVEDRFNASLGTYHDLLKIIKDKDFLDNE | |
| | ENEDILEDIVLTLTLFEDREMIEERLKTYA | |
| | HLFDDKVMKQLKRRRYTGWGRLSRKLINGI | |
| | RDKQSGKTILDFLKSDGFANRNFMQLIHDD | |
| | SLTFKEDIQKAQVSGQGDSLHEHIANLAGS | |
| | PAIKKGILQTVKVVDELVKVMGRHKPENIV | |
| | IEMARENQTTQKGQKNSRERMKRIEEGIKE | |
| | LGSQILKEHPVENTQLQNEKLYLYYLQNGR | |
| | DMYVDQELDINRLSDYDVDHIVPQSFLKDD | |
| | SIDNKVLTRSDKNRGKSDNVPSEEVVKKMK | |
| | NYWRQLLNAKLITQRKFDNLTKAERGGLSE | |
| | LDKAGFIKRQLVETRQITKHVAQILDSRMN | |
| | TKYDENDKLIREVKVITLKSKLVSDFRKDF | |
| | QFYKVREINNYHHAHDAYLNAVVGTALIKK | |
| | YPKLESEFVYGDYKVYDVRKMIAKSEQEIG | |
| | KATAKYFFYSNIMNFFKTEITLANGEIRKR | |
| | PLIETNGETGEIVWDKGRDFATVRKVLSMP | |
| | QVNIVKKTEVQTGGFSKESILPKRNSDKLI | |
| | ARKKDWDPKKYGGFDSPTVAYSVLVVAKVE | |
| | KGKSKKLKSVKELLGITIMERSSFEKNPID | |
| | FLEAKGYKEVKKDLIIKLPKYSLFELENGR | |
| | KRMLASAGELQKGNELALPSKYVNFLYLAS | |
| | HYEKLKGS | |
| CP1300 | KPIREQAENIIHLFTLTNLGAPAAFKYFDT | 117 |
| | TIDRKRYTSTKEVLDATLIHQSITGLYETR | |
| | IDLSQLGGDGGSGGSGGSGGSGGSGGSGGD | |
| | KKYSIGLAIGTNSVGWAVITDEYKVPSKKF | |
| | KVLGNTDRHSIKKNLIGALLFDSGETAEAT | |
| | RLKRTARRRYTRRKNRICYLQEIFSNEMAK | |
| | VDDSFFHRLEESFLVEEDKKHERHPIFGNI | |
| | VDEVAYHEKYPTIYHLRKKLVDSTDKADLR | |
| | LIYLALAHMIKFRGHFLIEGDLNPDNSDVD | |
| | KLFIQLVQTYNQLFEENPINASGVDAKAIL | |
| | SARLSKSRRLENLIAQLPGEKKNGLFGNLI | |
| | ALSLGLTPNFKSNFDLAEDAKLQLSKDTYD | |
| | DDLDNLLAQIGDQYADLFLAAKNLSDAILL | |
| | SDILRVNTEITKAPLSASMIKRYDEHHQDL | |
| | TLLKALVRQQLPEKYKEIFFDQSKNGYAGY | |
| | IDGGASQEEFYKFIKPILEKMDGTEELLVK | |
| | LNREDLLRKQRTFDNGSIPHQIHLGELHAI | |
| | LRRQEDFYPFLKDNREKIEKILTFRIPYYV | |
| | GPLARGNSRFAWMTRKSEETITPWNFEEVV | |
| | DKGASAQSFIERMTNFDKNLPNEKVLPKHS | |
| | LLYEYFTVYNELTKVKYVTEGMRKPAFLSG | |
| | EQKKAIVDLLFKTNRKVTVKQLKEDYFKKI | |
| | ECFDSVEISGVEDRFNASLGTYHDLLKIIK | |
| | DKDFLDNEENEDILEDIVLTLTLFEDREMI | |
| | EERLKTYAHLFDDKVMKQLKRRRYTGWGRL | |
| | SRKLINGIRDKQSGKTILDFLKSDGFANRN | |
| | FMQLIHDDSLTFKEDIQKAQVSGQGDSLHE | |
| | HIANLAGSPAIKKGILQTVKVVDELVKVMG | |
| | RHKPENIVIEMARENQTTQKGQKNSRERMK | |
| | RIEEGIKELGSQILKEHPVENTQLQNEKLY | |
| | LYYLQNGRDMYVDQELDINRLSDYDVDHIV | |
| | PQSFLKDDSIDNKVLTRSDKNRGKSDNVPS | |
| | EEVVKKMKNYWRQLLNAKLITQRKFDNLTK | |
| | AERGGLSELDKAGFIKRQLVETRQITKHVA | |
| | QILDSRMNTKYDENDKLIREVKVITLKSKL | |
| | VSDFRKDFQFYKVREINNYHHAHDAYLNAV | |
| | VGTALIKKYPKLESEFVYGDYKVYDVRKMI | |
| | AKSEQEIGKATAKYFFYSNIMNFFKTEITL | |
| | ANGEIRKRPLIETNGETGEIVWDKGRDFAT | |
| | VRKVLSMPQVNIVKKTEVQTGGFSKESILP | |
| | KRNSDKLIARKKDWDPKKYGGFDSPTVAYS | |
| | VLVVAKVEKGKSKKLKSVKELLGITIMERS | |
| | SFEKNPIDFLEAKGYKEVKKDLIIKLPKYS | |
| | LFELENGRKRMLASAGELQKGNELALPSKY | |
| | VNFLYLASHYEKLKGSPEDNEQKQLFVEQH | |
| | KHYLDEIIEQISEFSKRVILADANLDKVLS | |
| | AYNKHRD | |

The Cas9 circular permutants that may be useful in the base editing constructs described herein. Exemplary C-terminal fragments of Cas9, based on the Cas9 of SEQ ID NO: 58, which may be rearranged to an N-terminus of Cas9, are provided below. It should be appreciated that such C-terminal fragments of Cas9 are exemplary and are not meant to be limiting. These exemplary CP-Cas9 fragments have the following sequences:

| | | SEQ |
|---|---|---|
| | | ID |
| CP name | Sequence | NO: |
| CP1012 | DYKVYDVRKMIAKSEQEIGKATAKYFFYSNIMN | 118 |
| C- | FFKTEITLANGEIRKRPLIETNGETGEIVWDKG | |
| terminal | RDFATVRKVLSMPQVNIVKKTEVQTGGFSKESI | |
| fragment | LPKRNSDKLIARKKDWDPKKYGGFDSPTVAYSV | |
| | LVVAKVEKGKSKKLKSVKELLGITIMERSSFEK | |
| | NPIDFLEAKGYKEVKKDLIIKLPKYSLFELENG | |
| | RKRMLASAGELQKGNELALPSKYVNFLYLASHY | |
| | EKLKGSPEDNEQKQLFVEQHKHYLDEIIEQISE | |
| | FSKRVILADANLDKVLSAYNKHRDKPIREQAEN | |
| | IIHLFTLTNLGAPAAFKYFDTTIDRKRYTSTKE | |
| | VLDATLIHQSITGLYETRIDLSQLGGD | |
| CP1028 | EIGKATAKYFFYSNIMNFFKTEITLANGEIRKR | 119 |
| C- | PLIETNGETGEIVWDKGRDFATVRKVLSMPQVN | |
| terminal | IVKKTEVQTGGFSKESILPKRNSDKLIARKKDW | |
| fragment | DPKKYGGFDSPTVAYSVLVVAKVEKGKSKKLKS | |
| | VKELLGITIMERSSFEKNPIDFLEAKGYKEVKK | |
| | DLIIKLPKYSLFELENGRKRMLASAGELQKGNE | |
| | LALPSKYVNFLYLASHYEKLKGSPEDNEQKQLF | |
| | VEQHKHYLDEIIEQISEFSKRVILADANLDKVL | |
| | SAYNKHRDKPIREQAENIIHLFTLTNLGAPAAF | |
| | KYFDTTIDRKRYTSTKEVLDATLIHQSITGLYE | |
| | TRIDLSQLGGD | |
| CP1041 | NIMNFFKTEITLANGEIRKRPLIETNGETGEIV | 120 |
| C- | WDKGRDFATVRKVLSMPQVNIVKKTEVQTGGFS | |
| terminal | KESILPKRNSDKLIARKKDWDPKKYGGFDSPTV | |
| fragment | AYSVLVVAKVEKGKSKKLKSVKELLGITIMERS | |
| | SFEKNPIDFLEAKGYKEVKKDLIIKLPKYSLFE | |
| | LENGRKRMLASAGELQKGNELALPSKYVNFLYL | |
| | ASHYEKLKGSPEDNEQKQLFVEQHKHYLDEIIE | |
| | QISEFSKRVILADANLDKVLSAYNKHRDKPIRE | |
| | QAENIIHLFTLTNLGAPAAFKYFDTTIDRKRYT | |
| | STKEVLDATLIHQSITGLYETRIDLSQLGGD | |
| CP1249 | PEDNEQKQLFVEQHKHYLDEIIEQISEFSKRVI | 121 |
| C- | LADANLDKVLSAYNKHRDKPIREQAENIIHLFT | |
| terminal | LTNLGAPAAFKYFDTTIDRKRYTSTKEVLDATL | |
| fragment | IHQSITGLYETRIDLSQLGGD | |
| CP1300 | KPIREQAENIIHLFTLTNLGAPAAFKYFDTTID | 122 |
| C- | RKRYTSTKEVLDATLIHQSITGLYETRIDLSQL | |
| terminal | GGD | |
| fragment | | |

(10) Cas9 Variants with Modified PAM Specificities

The base editors of the present disclosure may also comprise Cas9 variants with modified PAM specificities. For example, the base editors described herein may utilize any naturally occurring or engineered variant of SpCas9 having expanded and/or relaxed PAM specificities which are described in the literature, including in Nishimasu et al., “Engineered CRISPR-Cas9 nuclease with expanded targeting space,” Science, 2018, 361: 1259-1262; Chatterjee et al., “Robust Genome Editing of Single-Base PAM Targets with Engineered ScCas9 Variants,” BioRxiv, Apr. 26, 2019Some aspects of this disclosure provide Cas9 proteins that exhibit activity on a target sequence that does not comprise the canonical PAM (5′-NGG-3′, where N is A, C, G, or T) at its 3′-end. In some embodiments, the Cas9 protein exhibits activity on a target sequence comprising a 5′-NGG-3′ PAM sequence at its 3′-end. In some embodiments, the Cas9 protein exhibits activity on a target sequence comprising a 5′-NNG-3′ PAM sequence at its 3′-end. In some embodiments, the Cas9 protein exhibits activity on a target sequence comprising a 5′-NNA-3′ PAM sequence at its 3′-end. In some embodiments, the Cas9 protein exhibits activity on a target sequence comprising a 5′-NNC-3′ PAM sequence at its 3′-end. In some embodiments, the Cas9 protein exhibits activity on a target sequence comprising a 5′-NNT-3′ PAM sequence at its 3′-end. In some embodiments, the Cas9 protein exhibits activity on a target sequence comprising a 5′-NGT-3′ PAM sequence at its 3′-end. In some embodiments, the Cas9 protein exhibits activity on a target sequence comprising a 5′-NGA-3′ PAM sequence at its 3′-end. In some embodiments, the Cas9 protein exhibits activity on a target sequence comprising a 5′-NGC-3′ PAM sequence at its 3′-end. In some embodiments, the Cas9 protein exhibits activity on a target sequence comprising a 5′-NAA-3′ PAM sequence at its 3′-end. In some embodiments, the Cas9 protein exhibits activity on a target sequence comprising a 5′-NAC-3′ PAM sequence at its 3′-end. In some embodiments, the Cas9 protein exhibits activity on a target sequence comprising a 5′-NAT-3′ PAM sequence at its 3′-end. In still other embodiments, the Cas9 protein exhibits activity on a target sequence comprising a 5′-NAG-3′ PAM sequence at its 3′-end.

It should be appreciated that any of the amino acid mutations described herein, (e.g., A262T) from a first amino acid residue (e.g., A) to a second amino acid residue (e.g., T) may also include mutations from the first amino acid residue to an amino acid residue that is similar to (e.g., conserved) the second amino acid residue. For example, mutation of an amino acid with a hydrophobic side chain (e.g., alanine, valine, isoleucine, leucine, methionine, phenylalanine, tyrosine, or tryptophan) may be a mutation to a second amino acid with a different hydrophobic side chain (e.g., alanine, valine, isoleucine, leucine, methionine, phenylalanine, tyrosine, or tryptophan). For example, a mutation of an alanine to a threonine (e.g., a A262T mutation) may also be a mutation from an alanine to an amino acid that is similar in size and chemical properties to a threonine, for example, serine. As another example, mutation of an amino acid with a positively charged side chain (e.g., arginine, histidine, or lysine) may be a mutation to a second amino acid with a different positively charged side chain (e.g., arginine, histidine, or lysine). As another example, mutation of an amino acid with a polar side chain (e.g., serine, threonine, asparagine, or glutamine) may be a mutation to a second amino acid with a different polar side chain (e.g., serine, threonine, asparagine, or glutamine). Additional similar amino acid pairs include, but are not limited to, the following: phenylalanine and tyrosine; asparagine and glutamine; methionine and cysteine; aspartic acid and glutamic acid; and arginine and lysine. The skilled artisan would recognize that such conservative amino acid substitutions will likely have minor effects on protein structure and are likely to be well tolerated without compromising function. In some embodiments, any amino of the amino acid mutations provided herein from one amino acid to a threonine may be an amino acid mutation to a serine. In some embodiments, any amino of the amino acid mutations provided herein from one amino acid to an arginine may be an amino acid mutation to a lysine. In some embodiments, any amino of the amino acid mutations provided herein from one amino acid to an isoleucine, may be an amino acid mutation to an alanine, valine, methionine, or leucine. In some embodiments, any amino of the amino acid mutations provided herein from one amino acid to a lysine may be an amino acid mutation to an arginine. In some embodiments, any amino of the amino acid mutations provided herein from one amino acid to an aspartic acid may be an amino acid mutation to a glutamic acid or asparagine. In some embodiments, any amino of the amino acid mutations provided herein from one amino acid to a valine may be an amino acid mutation to an alanine, isoleucine, methionine, or leucine. In some embodiments, any amino of the amino acid mutations provided herein from one amino acid to a glycine may be an amino acid mutation to an alanine. It should be appreciated, however, that additional conserved amino acid residues would be recognized by the skilled artisan and any of the amino acid mutations to other conserved amino acid residues are also within the scope of this disclosure.

In some embodiments, the Cas9 protein comprises a combination of mutations that exhibit activity on a target sequence comprising a 5′-NAA-3′ PAM sequence at its 3′-end. In some embodiments, the combination of mutations are present in any one of the clones listed in Table 1. In some embodiments, the combination of mutations are conservative mutations of the clones listed in Table 1. In some embodiments, the Cas9 protein comprises the combination of mutations of any one of the Cas9 clones listed in Table 1.

TABLE 1
NAA PAM Clones
Mutations from wild-type SpCas9 (e.g., SEQ ID NO: 59)
D177N, K218R, D614N, D1135N, P1137S, E1219V, A1320V, A1323D, R1333K
D177N, K218R, D614N, D1135N, E1219V, Q1221H, H1264Y, A1320V, R1333K
A10T, I322V, S409I, E427G, G715C, D1135N, E1219V, Q1221H, H1264Y, A1320V, R1333K
A367T, K710E, R1114G, D1135N, P1137S, E1219V, Q1221H, H1264Y, A1320V, R1333K
A10T, I322V, S409I, E427G, R753G, D861N, D1135N, K1188R, E1219V, Q1221H, H1264H, A1320V,
R1333K
A10T, I322V, S4091, E427G, R654L, V743I, R753G, M1021T, D1135N, D1180G, K1211R, E1219V,
Q1221H, H1264Y, A1320V, R1333K
A10T, I322V, S409I, E427G, V743I, R753G, E762G, D1135N, D1180G, K1211R, E1219V, Q1221H,
H1264Y, A1320V, R1333K
A10T, I322V, S409I, E427G, R753G, D1135N, D1180G, K1211R, E1219V, Q1221H, H1264Y, S1274R,
A1320V, R1333K
A10T, I322V, S409I, E427G, A589S, R753G, D1135N, E1219V, Q1221H, H1264H, A1320V, R1333K
A10T, I322V, S409I, E427G, R753G, E757K, G865G, D1135N, E1219V, Q1221H, H1264Y, A1320V,
R1333K
A10T, I322V, S4091, E427G, R654L, R753G, E757K, D1135N, E1219V, Q1221H, H1264Y, A1320V,
R1333K
A10T, I322V, S409I, E427G, K599R, M631A, R654L, K673E, V743I, R753G, N758H, E762G, D1135N,
D1180G, E1219V, Q1221H, Q1256R, H1264Y, A1320V, A1323D, R1333K
A10T, 1322V, S409I, E427G, R654L, K673E, V743I, R753G, E762G, N869S, N1054D, R1114G,
D1135N, D1180G, E1219V, Q1221H, H1264Y, A1320V, A1323D, R1333K
A10T, I322V, S4091, E427G, R654L, L727I, V743I, R753G, E762G, R859S, N946D, F1134L, D1135N,
D1180G, E1219V, Q1221H, H1264Y, N1317T, A1320V, A1323D, R1333K
A10T, 1322V, S409I, E427G, R654L, K673E, V743I, R753G, E762G, N803S, N869S, Y1016D, G1077D,
R1114G, F1134L, D1135N, D1180G, E1219V, Q1221H, H1264Y, V1290G, L1318S, A1320V, A1323D,
R1333K
A10T, I322V, S4091, E427G, R654L, K673E, V743I, R753G, E762G, N803S, N869S, Y1016D, G1077D,
R1114G, F1134L, D1135N, K1151E, D1180G, E1219V, Q1221H, H1264Y, V1290G, L1318S, A1320V,
R1333K
A10T, I322V, S409I, E427G, R654L, K673E, V743I, R753G, E762G, N803S, N869S, Y1016D, G1077D,
R1114G, F1134L, D1135N, D1180G, E1219V, Q1221H, H1264Y, V1290G, L1318S, A1320V, A1323D,
R1333K
A10T, I322V, S4091, E427G, R654L, K673E, F693L, V743I, R753G, E762G, N803S, N869S, L921P,
Y1016D, G1077D, F1080S, R1114G, D1135N, D1180G, E1219V, Q1221H, H1264Y, L1318S, A1320V,
A1323D, R1333K
A10T, I322V, S4091, E427G, E630K, R654L, K673E, V743I, R753G, E762G, Q768H, N803S, N869S,
Y1016D, G1077D, R1114G, F1134L, D1135N, D1180G, E1219V, Q1221H, H1264Y, L1318S, A1320V,
R1333K
A10T, I322V, S4091, E427G, R654L, K673E, F693L, V743I, R753G, E762G, Q768H, N803S, N869S,
Y1016D, G1077D, R1114G, F1134L, D1135N, D1180G, E1219V, Q1221H, G1223S, H1264Y, L1318S,
A1320V, R1333K
A10T, I322V, S4091, E427G, R654L, K673E, F693L, V743I, R753G, E762G, N803S, N869S, L921P,
Y1016D, G1077D, F1801S, R1114G, D1135N, D1180G, E1219V, Q1221H, H1264Y, L1318S, A1320V,
A1323D, R1333K
A10T, I322V, S409I, E427G, R654L, V743I, R753G, M1021T, D1135N, D1180G, K1211R, E1219V,
Q1221H, H1264Y, A1320V, R1333K
A10T, I322V, S4091, E427G, R654L, K673E, V743I, R753G, E762G, M673I, N803S, N869S, G1077D,
R1114G, D1135N, V1139A, D1180G, E1219V, Q1221H, A1320V, R1333K
A10T, I322V, S4091, E427G, R654L, K673E, V743I, R753G, E762G, N803S, N869S, R1114G, D1135N,
E1219V, Q1221H, A1320V, R1333K

In some embodiments, the Cas9 protein comprises an amino acid sequence that is at least 80% identical to the amino acid sequence of a Cas9 protein as provided by any one of the variants of Table 1. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the variants of Table 1.

In some embodiments, the Cas9 protein exhibits an increased activity on a target sequence that does not comprise the canonical PAM (5′-NGG-3′) at its 3′ end as compared to Streptococcus pyogenes Cas9 as provided by SEQ ID NO: 59. In some embodiments, the Cas9 protein exhibits an activity on a target sequence having a 3′ end that is not directly adjacent to the canonical PAM sequence (5′-NGG-3′) that is at least 5-fold increased as compared to the activity of Streptococcus pyogenes Cas9 as provided by SEQ ID NO: 59 on the same target sequence. In some embodiments, the Cas9 protein exhibits an activity on a target sequence that is not directly adjacent to the canonical PAM sequence (5′-NGG-3′) that is at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the activity of Streptococcus pyogenes as provided by SEQ ID NO: 59 on the same target sequence. In some embodiments, the 3 end of the target sequence is directly adjacent to an AAA, GAA, CAA, or TAA sequence. In some embodiments, the Cas9 protein comprises a combination of mutations that exhibit activity on a target sequence comprising a 5′-NAC-3′ PAM sequence at its 3′-end. In some embodiments, the combination of mutations are present in any one of the clones listed in Table 2. In some embodiments, the combination of mutations are conservative mutations of the clones listed in Table 2. In some embodiments, the Cas9 protein comprises the combination of mutations of any one of the Cas9 clones listed in Table 2.

TABLE 2
NAC PAM Clones
Mutations from wild-type SpCas9 (e.g., SEQ ID NO: 59)
T472I, R753G, K890E, D1332N, R1335Q, T1337N
I1057S, D1135N, P1301S, R1335Q, T1337N
T472I, R753G, D1332N, R1335Q, T1337N
D1135N, E1219V, D1332N, R1335Q, T1337N
T472I, R753G, K890E, D1332N, R1335Q, T1337N
I1057S, D1135N, P1301S, R1335Q, T1337N
T472I, R753G, D1332N, R1335Q, T1337N
T472I, R753G, Q771H, D1332N, R1335Q, T1337N
E627K, T638P, K652T, R753G, N803S, K959N, R1114G, D1135N, E1219V, D1332N, R1335Q, T1337N
E627K, T638P, K652T, R753G, N803S, K959N, R1114G, D1135N, K1156E, E1219V, D1332N, R1335Q,
T1337N
E627K, T638P, V6471, R753G, N803S, K959N, G1030R, I1055E, R1114G, D1135N, E1219V, D1332N,
R1335Q, T1337N
E627K, E630G, T638P, V647A, G687R, N767D, N803S, K959N, R1114G, D1135N, E1219V, D1332G,
R1335Q, T1337N
E627K, T638P, R753G, N803S, K959N, R1114G, D1135N, E1219V, N1266H, D1332N, R1335Q, T1337N
E627K, T638P, R753G, N803S, K959N, I1057T, R1114G, D1135N, E1219V, D1332N, R1335Q, T1337N
E627K, T638P, R753G, N803S, K959N, R1114G, D1135N, E1219V, D1332N, R1335Q, T1337N
E627K, M631I, T638P, R753G, N803S, K959N, Y1036H, R1114G, D1135N, E1219V, D1251G, D1332G,
R1335Q, T1337N
E627K, T638P, R753G, N803S, V875I, K959N, Y1016C, R1114G, D1135N, E1219V, D1251G, D1332G,
R1335Q, T1337N, I1348V
K608R, E627K, T638P, V6471, R654L, R753G, N803S, T804A, K848N, V922A, K959N, R1114G,
D1135N, E1219V, D1332N, R1335Q, T1337N
K608R, E627K, T638P, V6471, R753G, N803S, V922A, K959N, K1014N, V1015A, R1114G, D1135N,
K1156N, E1219V, N1252D, D1332N, R1335Q, T1337N
K608R, E627K, R629G, T638P, V6471, A711T, R753G, K775R, K789E, N803S, K959N, V1015A,
Y1036H, R1114G, D1135N, E1219V, N1286H, D1332N, R1335Q, T1337N
K608R, E627K, T638P, V6471, T740A, R753G, N803S, K948E, K959N, Y1016S, R1114G, D1135N,
E1219V, N1286H, D1332N, R1335Q, T1337N
K608R, E627K, T638P, V6471, T740A, N803S, K948E, K959N, Y1016S, R1114G, D1135N, E1219V,
N1286H, D1332N, R1335Q, T1337N
I670S, K608R, E627K, E630G, T638P, V6471, R653K, R753G, I795L, K797N, N803S, K866R, K890N,
K959N, Y1016C, R1114G, D1135N, E1219V, D1332N, R1335Q, T1337N
K608R, E627K, T638P, V6471, T740A, G752R, R753G, K797N, N803S, K948E, K959N, V1015A,
Y1016S, R1114G, D1135N, E1219V, N1266H, D1332N, R1335Q, T1337N
I570T, A589V, K608R, E627K, T638P, V6471, R654L, Q716R, R753G, N803S, K948E, K959N, Y1016S,
R1114G, D1135N, E1207G, E1219V, N1234D, D1332N, R1335Q, T1337N
K608R, E627K, R629G, T638P, V6471, R654L, Q740R, R753G, N803S, K959N, N990S, T995S,
V1015A, Y1036D, R1114G, D1135N, E1207G, E1219V, N1234D, N1266H, D1332N, R1335Q, T1337N
I562F, V565D, 1570T, K608R, L625S, E627K, T638P, V6471, R654I, G752R, R753G, N803S, N808D,
K959N, M1021L, R1114G, D1135N, N1177S, N1234D, D1332N, R1335Q, T1337N
I562F, 1570T, K608R, E627K, T638P, V6471, R753G, E790A, N803S, K959N, V1015A, Y1036H,
R1114G, D1135N, D1180E, A1184T, E1219V, D1332N, R1335Q, T1337N
I570T, K608R, E627K, T638P, V6471, R654H, R753G, E790A, N803S, K959N, V1015A, R1114G,
D1127A, D1135N, E1219V, D1332N, R1335Q, T1337N
I570T, K608R, L625S, E627K, T638P, V6471, R654I, T703P, R753G, N803S, N808D, K959N, M1021L,
R1114G, D1135N, E1219V, D1332N, R1335Q, T1337N
I570S, K608R, E627K, E630G, T638P, V6471, R653K, R753G, I795L, N803S, K866R, K890N, K959N,
Y1016C, R1114G, D1135N, E1219V, D1332N, R1335Q, T1337N
I570T, K608R, E627K, T638P, V647I, R654H, R753G, E790A, N803S, K959N, V1016A, R1114G,
D1135N, E1219V, K1246E, D1332N, R1335Q, T1337N
K608R, E627K, T638P, V6471, R654L, K673E, R753G, E790A, N803S, K948E, K959N, R1114G,
D1127G, D1135N, D1180E, E1219V, N1286H, D1332N, R1335Q, T1337N
K608R, L625S, E627K, T638P, V6471, R654I, I670T, R753G, N803S, N808D, K959N, M1021L,
R1114G, D1135N, E1219V, N1286H, D1332N, R1335Q, T1337N
E627K, M631V, T638P, V647I, K710E, R753G, N803S, N808D, K948E, M1021L, R1114G, D1135N,
E1219V, D1332N, R1335Q, T1337N, S1338T, H1349R

In some embodiments, the Cas9 protein comprises an amino acid sequence that is at least 80% identical to the amino acid sequence of a Cas9 protein as provided by any one of the variants of Table 2. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the variants of Table 2.

In some embodiments, the Cas9 protein exhibits an increased activity on a target sequence that does not comprise the canonical PAM (5′-NGG-3′) at its 3′ end as compared to Streptococcus pyogenes Cas9 as provided by SEQ ID NO: 59. In some embodiments, the Cas9 protein exhibits an activity on a target sequence having a 3′ end that is not directly adjacent to the canonical PAM sequence (5′-NGG-3′) that is at least 5-fold increased as compared to the activity of Streptococcus pyogenes Cas9 as provided by SEQ ID NO: 59 on the same target sequence. In some embodiments, the Cas9 protein exhibits an activity on a target sequence that is not directly adjacent to the canonical PAM sequence (5′-NGG-3′) that is at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the activity of Streptococcus pyogenes as provided by SEQ ID NO: 59 on the same target sequence. In some embodiments, the 3′ end of the target sequence is directly adjacent to an AAC, GAC, CAC, or TAC sequence.

In some embodiments, the Cas9 protein comprises a combination of mutations that exhibit activity on a target sequence comprising a 5′-NAT-3′ PAM sequence at its 3′-end. In some embodiments, the combination of mutations are present in any one of the clones listed in Table 3. In some embodiments, the combination of mutations are conservative mutations of the clones listed in Table 3. In some embodiments, the Cas9 protein comprises the combination of mutations of any one of the Cas9 clones listed in Table 3.

TABLE 3
NAT PAM Clones
Mutations from wild-type SpCas9 (e.g., SEQ ID NO: 59)
K961E, H985Y, D1135N, K1191N, E1219V, Q1221H, A1320A, P1321S, R1335L
D1135N, G1218S, E1219V, Q1221H, P1249S, P1321S, D1322G, R1335L
V743I, R753G, E790A, D1135N, G1218S, E1219V, Q1221H, A1227V, P1249S, N1286K, A1293T,
P1321S, D1322G, R1335L, T1339I
F575S, M631L, R654L, V748I, V743I, R753G, D853E, V922A, R1114G D1135N, G1218S, E1219V,
Q1221H, A1227V, P1249S, N1286K, A1293T, P1321S, D1322G, R1335L, T1339I
F575S, M631L, R654L, R664K, R753G, D853E, V922A, R1114G D1135N, D1180G, G1218S, E1219V,
Q1221H, P1249S, N1286K, P1321S, D1322G, R1335L
M631L, R654L, R753G, K797E, D853E, V922A, D1012A, R1114G D1135N, G1218S, E1219V, Q1221H,
P1249S, N1317K, P1321S, D1322G, R1335L
F575S, M631L, R654L, R664K, R753G, D853E, V922A, R1114G, Y1131C, D1135N, D1180G, G1218S,
E1219V, Q1221H, P1249S, P1321S, D1322G, R1335L
F575S, M631L, R654L, R664K, R753G, D853E, V922A, R1114G, Y1131C, D1135N, D1180G, G1218S,
E1219V, Q1221H, P1249S, P1321S, D1322G, R1335L
F575S, D596Y, M631L, R654L, R664K, R753G, D853E, V922A, R1114G, Y1131C, D1135N, D1180G,
G1218S, E1219V, Q1221H, P1249S, Q1256R, P1321S, D1322G, R1335L
F575S, M631L, R654L, R664K, K710E, V750A, R753G, D853E, V922A, R1114G, Y1131C, D1135N,
D1180G, G1218S, E1219V, Q1221H, P1249S, P1321S, D1322G, R1335L
F575S, M631L, K649R, R654L, R664K, R753G, D853E, V922A, R1114G, Y1131C, D1135N, K1156E,
D1180G, G1218S, E1219V, Q1221H, P1249S, P1321S, D1322G, R1335L
F575S, M631L, R654L, R664K, R753G, D853E, V922A, R1114G, Y1131C, D1135N, D1180G, G1218S,
E1219V, Q1221H, P1249S, P1321S, D1322G, R1335L
F575S, M631L, R654L, R664K, R753G, D853E, V922A, I1057G, R1114G, Y1131C, D1135N, D1180G,
G1218S, E1219V, Q1221H, P1249S, N1308D, P1321S, D1322G, R1335L
M631L, R654L, R753G, D853E, V922A, R1114G, Y1131C, D1135N, E1150V, D1180G, G1218S,
E1219V, Q1221H, P1249S, P1321S, D1332G, R1335L
M631L, R654L, R664K, R753G, D853E, I1057V, Y1131C, D1135N, D1180G, G1218S, E1219V,
Q1221H, P1249S, P1321S, D1332G, R1335L
M631L, R654L, R664K, R753G, I1057V, R1114G, Y1131C, D1135N, D1180G, G1218S, E1219V,
Q1221H, P1249S, P1321S, D1332G, R1335L

The above description of various napDNAbps which can be used in connection with the presently disclose base editors is not meant to be limiting in any way. The base editors may comprise the canonical SpCas9, or any ortholog Cas9 protein, or any variant Cas9 protein—including any naturally occurring variant, mutant, or otherwise engineered version of Cas9—that is known or which can be made or evolved through a directed evolutionary or otherwise mutagenic process. In various embodiments, the Cas9 or Cas9 variants have a nickase activity, i.e., only cleave of strand of the target DNA sequence. In other embodiments, the Cas9 or Cas9 variants have inactive nucleases, i.e., are “dead” Cas9 proteins. Other variant Cas9 proteins that may be used are those having a smaller molecular weight than the canonical SpCas9 (e.g., for easier delivery) or having modified or rearranged primary amino acid structure (e.g., the circular permutant formats). The base editors described herein may also comprise Cas9 equivalents, including Cas12a/Cpf1 and Cas12b proteins which are the result of convergent evolution. The napDNAbps used herein (e.g., SpCas9, Cas9 variant, or Cas9 equivalents) may also may also contain various modifications that alter/enhance their PAM specificities. Lastly, the application contemplates any Cas9, Cas9 variant, or Cas9 equivalent which has at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity to a reference Cas9 sequence, such as a references SpCas9 canonical sequences or a reference Cas9 equivalent (e.g., Cas12a/Cpf1).

In a particular embodiment, the Cas9 variant having expanded PAM capabilities is SpCas9 (H840A) VRQR, having the following amino acid sequence (with the V, R, Q, R substitutions relative to the SpCas9 (H840A) of SEQ ID NO: 123 shown in bold underline. In addition, the methionine residue in SpCas9 (H840) was removed for SpCas9 (H840A) VRQR) (“SpCas9-VRQR”). This SpCas9 variant possesses an altered PAM-specificity which recognizes a PAM of 5′-NGA-3′ instead of the canonical PAM of 5′-NGG-3′:

SpCas9-VRQR
(SEQ ID NO: 123)
DKKYSIGLDIGTNSVGWAVITDEYKVPSKKFKVLGNTDRHSI
KKNLIGALLFDSGETAEATRLKRTARRRYTRRKNRICYLQEI
FSNEMAKVDDSFFHRLEESFLVEEDKKHERHPIFGNIVDEVA
YHEKYPTIYHLRKKLVDSTDKADLRLIYLALAHMIKFRGHFL
IEGDLNPDNSDVDKLFIQLVQTYNQLFEENPINASGVDAKAI
LSARLSKSRRLENLIAQLPGEKKNGLFGNLIALSLGLTPNFK
SNFDLAEDAKLQLSKDTYDDDLDNLLAQIGDQYADLFLAAKN
LSDAILLSDILRVNTEITKAPLSASMIKRYDEHHQDLTLLKA
LVRQQLPEKYKEIFFDQSKNGYAGYIDGGASQEEFYKFIKPI
LEKMDGTEELLVKLNREDLLRKQRTFDNGSIPHQIHLGELHA
ILRRQEDFYPFLKDNREKIEKILTFRIPYYVGPLARGNSRFA
WMTRKSEETITPWNFEEVVDKGASAQSFIERMTNFDKNLPNE
KVLPKHSLLYEYFTVYNELTKVKYVTEGMRKPAFLSGEQKKA
IVDLLFKTNRKVTVKQLKEDYFKKIECFDSVEISGVEDRFNA
SLGTYHDLLKIIKDKDFLDNEENEDILEDIVLTLTLFEDREM
IEERLKTYAHLFDDKVMKQLKRRRYTGWGRLSRKLINGIRDK
QSGKTILDFLKSDGFANRNFMQLIHDDSLTFKEDIQKAQVSG
QGDSLHEHIANLAGSPAIKKGILQTVKVVDELVKVMGRHKPE
NIVIEMARENQTTQKGQKNSRERMKRIEEGIKELGSQILKEH
PVENTQLQNEKLYLYYLQNGRDMYVDQELDINRLSDYDVDAI
VPQSFLKDDSIDNKVLTRSDKNRGKSDNVPSEEVVKKMKNYW
RQLLNAKLITQRKFDNLTKAERGGLSELDKAGFIKRQLVETR
QITKHVAQILDSRMNTKYDENDKLIREVKVITLKSKLVSDFR
KDFQFYKVREINNYHHAHDAYLNAVVGTALIKKYPKLESEFV
YGDYKVYDVRKMIAKSEQEIGKATAKYFFYSNIMNFFKTEIT
LANGEIRKRPLIETNGETGEIVWDKGRDFATVRKVLSMPQVN
IVKKTEVQTGGFSKESILPKRNSDKLIARKKDWDPKKYGGFV
SPTVAYSVLVVAKVEKGKSKKLKSVKELLGITIMERSSFEKN
PIDFLEAKGYKEVKKDLIIKLPKYSLFELENGRKRMLASARE
LQKGNELALPSKYVNFLYLASHYEKLKGSPEDNEQKQLFVEQ
HKHYLDEIIEQISEFSKRVILADANLDKVLSAYNKHRDKPIR
EQAENIIHLFTLTNLGAPAAFKYFDTTIDRKQYRSTKEVLDA
TLIHQSITGLYETRIDLSQLGGD

In another particular embodiment, the Cas9 variant having expanded PAM capabilities is SpCas9 (H840A) VQR, having the following amino acid sequence (with the V, Q, R substitutions relative to the SpCas9 (H840A) of SEQ ID NO: 124 show in bold underline. In addition, the methionine residue in SpCas9 (H840) was removed for SpCas9 (H840A) VRQR) (“SpCas9-VQR”). This SpCas9 variant possesses an altered PAM-specificity which recognizes a PAM of 5′-NGA-3′ instead of the canonical PAM of 5′-NGG-3′:

SpCas9-VQR
(SEQ ID NO: 124)
DKKYSIGLDIGTNSVGWAVITDEYKVPSKKFKVLGNTDRHSI
KKNLIGALLFDSGETAEATRLKRTARRRYTRRKNRICYLQEI
FSNEMAKVDDSFFHRLEESFLVEEDKKHERHPIFGNIVDEVA
YHEKYPTIYHLRKKLVDSTDKADLRLIYLALAHMIKFRGHFL
IEGDLNPDNSDVDKLFIQLVQTYNQLFEENPINASGVDAKAI
LSARLSKSRRLENLIAQLPGEKKNGLFGNLIALSLGLTPNFK
SNFDLAEDAKLQLSKDTYDDDLDNLLAQIGDQYADLFLAAKN
LSDAILLSDILRVNTEITKAPLSASMIKRYDEHHQDLTLLKA
LVRQQLPEKYKEIFFDQSKNGYAGYIDGGASQEEFYKFIKPI
LEKMDGTEELLVKLNREDLLRKQRTFDNGSIPHQIHLGELHA
ILRRQEDFYPFLKDNREKIEKILTFRIPYYVGPLARGNSRFA
WMTRKSEETITPWNFEEVVDKGASAQSFIERMTNFDKNLPNE
KVLPKHSLLYEYFTVYNELTKVKYVTEGMRKPAFLSGEQKKA
IVDLLFKTNRKVTVKQLKEDYFKKIECFDSVEISGVEDRFNA
SLGTYHDLLKIIKDKDFLDNEENEDILEDIVLTLTLFEDREM
IEERLKTYAHLFDDKVMKQLKRRRYTGWGRLSRKLINGIRDK
QSGKTILDFLKSDGFANRNFMQLIHDDSLTFKEDIQKAQVSG
QGDSLHEHIANLAGSPAIKKGILQTVKVVDELVKVMGRHKPE
NIVIEMARENQTTQKGQKNSRERMKRIEEGIKELGSQILKEH
PVENTQLQNEKLYLYYLQNGRDMYVDQELDINRLSDYDVDAI
VPQSFLKDDSIDNKVLTRSDKNRGKSDNVPSEEVVKKMKNYW
RQLLNAKLITQRKFDNLTKAERGGLSELDKAGFIKRQLVETR
QITKHVAQILDSRMNTKYDENDKLIREVKVITLKSKLVSDFR
KDFQFYKVREINNYHHAHDAYLNAVVGTALIKKYPKLESEFV
YGDYKVYDVRKMIAKSEQEIGKATAKYFFYSNIMNFFKTEIT
LANGEIRKRPLIETNGETGEIVWDKGRDFATVRKVLSMPQVN
IVKKTEVQTGGFSKESILPKRNSDKLIARKKDWDPKKYGGFV
SPTVAYSVLVVAKVEKGKSKKLKSVKELLGITIMERSSFEKN
PIDFLEAKGYKEVKKDLIIKLPKYSLFELENGRKRMLASAGE
LQKGNELALPSKYVNFLYLASHYEKLKGSPEDNEQKQLFVEQ
HKHYLDEIIEQISEFSKRVILADANLDKVLSAYNKHRDKPIR
EQAENIIHLFTLTNLGAPAAFKYFDTTIDRKQYRSTKEVLDA
TLIHQSITGLYETRIDLSQLGGD

In another particular embodiment, the Cas9 variant having expanded PAM capabilities is SpCas9 (H840A) VRER, having the following amino acid sequence (with the V, R, E, R substitutions relative to the SpCas9 (H840A) of SEQ ID NO: 125 are shown in bold underline. In addition, the methionine residue in SpCas9 (H840) was removed for SpCas9 (H840A) VRER) (“SpCas9-VRER”). This SpCas9 variant possesses an altered PAM-specificity which recognizes a PAM of 5′-NGCG-3′ instead of the canonical PAM of 5′-NGG-3′:

SpCas9-VRER
(SEQ ID NO: 125)
DKKYSIGLDIGTNSVGWAVITDEYKVPSKKFKVLGNTDRHSI
KKNLIGALLFDSGETAEATRLKRTARRRYTRRKNRICYLQEI
FSNEMAKVDDSFFHRLEESFLVEEDKKHERHPIFGNIVDEVA
YHEKYPTIYHLRKKLVDSTDKADLRLIYLALAHMIKFRGHFL
IEGDLNPDNSDVDKLFIQLVQTYNQLFEENPINASGVDAKAI
LSARLSKSRRLENLIAQLPGEKKNGLFGNLIALSLGLTPNFK
SNFDLAEDAKLQLSKDTYDDDLDNLLAQIGDQYADLFLAAKN
LSDAILLSDILRVNTEITKAPLSASMIKRYDEHHQDLTLLKA
LVRQQLPEKYKEIFFDQSKNGYAGYIDGGASQEEFYKFIKPI
LEKMDGTEELLVKLNREDLLRKQRTFDNGSIPHQIHLGELHA
ILRRQEDFYPFLKDNREKIEKILTFRIPYYVGPLARGNSRFA
WMTRKSEETITPWNFEEVVDKGASAQSFIERMTNFDKNLPNE
KVLPKHSLLYEYFTVYNELTKVKYVTEGMRKPAFLSGEQKKA
IVDLLFKTNRKVTVKQLKEDYFKKIECFDSVEISGVEDRFNA
SLGTYHDLLKIIKDKDFLDNEENEDILEDIVLTLTLFEDREM
IEERLKTYAHLFDDKVMKQLKRRRYTGWGRLSRKLINGIRDK
QSGKTILDFLKSDGFANRNFMQLIHDDSLTFKEDIQKAQVSG
QGDSLHEHIANLAGSPAIKKGILQTVKVVDELVKVMGRHKPE
NIVIEMARENQTTQKGQKNSRERMKRIEEGIKELGSQILKEH
PVENTQLQNEKLYLYYLQNGRDMYVDQELDINRLSDYDVDAI
VPQSFLKDDSIDNKVLTRSDKNRGKSDNVPSEEVVKKMKNYW
RQLLNAKLITQRKFDNLTKAERGGLSELDKAGFIKRQLVETR
QITKHVAQILDSRMNTKYDENDKLIREVKVITLKSKLVSDFR
KDFQFYKVREINNYHHAHDAYLNAVVGTALIKKYPKLESEFV
YGDYKVYDVRKMIAKSEQEIGKATAKYFFYSNIMNFFKTEIT
LANGEIRKRPLIETNGETGEIVWDKGRDFATVRKVLSMPQVN
IVKKTEVQTGGFSKESILPKRNSDKLIARKKDWDPKKYGGFV
SPTVAYSVLVVAKVEKGKSKKLKSVKELLGITIMERSSFEKN
PIDFLEAKGYKEVKKDLIIKLPKYSLFELENGRKRMLASARE
LQKGNELALPSKYVNFLYLASHYEKLKGSPEDNEQKQLFVEQ
HKHYLDEIIEQISEFSKRVILADANLDKVLSAYNKHRDKPIR
EQAENIIHLFTLTNLGAPAAFKYFDTTIDRKEYRSTKEVLDA
TLIHQSITGLYETRIDLSQLGGD

In yet particular embodiment, the Cas9 variant having expanded PAM capabilities is SpCas9-NG, as reported in Nishimasu et al., “Engineered CRISPR-Cas9 nuclease with expanded targeting space,” Science, 2018, 361: 1259-1262, which is incorporated herein by reference. SpCas9-NG (VRVRFRR), having the following amino acid sequence substitutions: R1335V, L1111R, D1135V, G1218R, E1219F, A1322R, and T1337R relative to the canonical SpCas9 sequence (SEQ ID NO: 59. This SpCas9 has a relaxed PAM specificity, i.e., with activity on a PAM of NGH (wherein H=A, T, or C). See Nishimasu et al., “Engineered CRISPR-Cas9 nuclease with expanded targeting space,” Science, 2018, 361: 1259-1262, which is incorporated herein by reference.

SpCas9-NG
(SEQ ID NO: 126)
MDKKYSIGLDIGTNSVGWAVITDEYKVPSKKFKVLGNTDRHS
IKKNLIGALLFDSGETAEATRLKRTARRRYTRRKNRICYLQE
IFSNEMAKVDDSFFHRLEESFLVEEDKKHERHPIFGNIVDEV
AYHEKYPTIYHLRKKLVDSTDKADLRLIYLALAHMIKFRGHF
LIEGDLNPDNSDVDKLFIQLVQTYNQLFEENPINASGVDAKA
ILSARLSKSRRLENLIAQLPGEKKNGLFGNLIALSLGLTPNF
KSNFDLAEDAKLQLSKDTYDDDLDNLLAQIGDQYADLFLAAK
NLSDAILLSDILRVNTEITKAPLSASMIKRYDEHHQDLTLLK
ALVRQQLPEKYKEIFFDQSKNGYAGYIDGGASQEEFYKFIKP
ILEKMDGTEELLVKLNREDLLRKQRTFDNGSIPHQIHLGELH
AILRRQEDFYPFLKDNREKIEKILTFRIPYYVGPLARGNSRF
AWMTRKSEETITPWNFEEVVDKGASAQSFIERMTNFDKNLPN
EKVLPKHSLLYEYFTVYNELTKVKYVTEGMRKPAFLSGEQKK
AIVDLLFKTNRKVTVKQLKEDYFKKIECFDSVEISGVEDRFN
ASLGTYHDLLKIIKDKDFLDNEENEDILEDIVLTLTLFEDRE
MIEERLKTYAHLFDDKVMKQLKRRRYTGWGRLSRKLINGIRD
KQSGKTILDFLKSDGFANRNFMQLIHDDSLTFKEDIQKAQVS
GQGDSLHEHIANLAGSPAIKKGILQTVKVVDELVKVMGRHKP
ENIVIEMARENQTTQKGQKNSRERMKRIEEGIKELGSQILKE
HPVENTQLQNEKLYLYYLQNGRDMYVDQELDINRLSDYDVDH
IVPQSFLKDDSIDNKVLTRSDKNRGKSDNVPSEEVVKKMKNY
WRQLLNAKLITQRKFDNLTKAERGGLSELDKAGFIKRQLVET
RQITKHVAQILDSRMNTKYDENDKLIREVKVITLKSKLVSDF
RKDFQFYKVREINNYHHAHDAYLNAVVGTALIKKYPKLESEF
VYGDYKVYDVRKMIAKSEQEIGKATAKYFFYSNIMNFFKTEI
TLANGEIRKRPLIETNGETGEIVWDKGRDFATVRKVLSMPQV
NIVKKTEVQTGGFSKESIRPKRNSDKLIARKKDWDPKKYGGF
VSPTVAYSVLVVAKVEKGKSKKLKSVKELLGITIMERSSFEK
NPIDFLEAKGYKEVKKDLIIKLPKYSLFELENGRKRMLASAR
FLQKGNELALPSKYVNFLYLASHYEKLKGSPEDNEQKQLFVE
QHKHYLDEIIEQISEFSKRVILADANLDKVLSAYNKHRDKPI
REQAENIIHLFTLTNLGAPRAFKYFDTTIDRKVYRSTKEVLD
ATLIHQSITGLYETRIDLSQLGGD

In addition, any available methods may be utilized to obtain or construct a variant or mutant Cas9 protein. The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which is the normal result of a mutation that reduces or abolishes a protein activity. Most loss-of-function mutations are recessive, because in a heterozygote the second chromosome copy carries an unmutated version of the gene coding for a fully functional protein whose presence compensates for the effect of the mutation. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. For example, a mutation might lead to one or more genes being expressed in the wrong tissues, these tissues gaining functions that they normally lack. Because of their nature, gain-of-function mutations are usually dominant.

Mutations can be introduced into a reference Cas9 protein using site-directed mutagenesis. Older methods of site-directed mutagenesis known in the art rely on sub-cloning of the sequence to be mutated into a vector, such as an M13 bacteriophage vector, that allows the isolation of single-stranded DNA template. In these methods, one anneals a mutagenic primer (i.e., a primer capable of annealing to the site to be mutated but bearing one or more mismatched nucleotides at the site to be mutated) to the single-stranded template and then polymerizes the complement of the template starting from the 3′ end of the mutagenic primer. The resulting duplexes are then transformed into host bacteria and plaques are screened for the desired mutation. More recently, site-directed mutagenesis has employed PCR methodologies, which have the advantage of not requiring a single-stranded template. In addition, methods have been developed that do not require sub-cloning. Several issues must be considered when PCR-based site-directed mutagenesis is performed. First, in these methods it is desirable to reduce the number of PCR cycles to prevent expansion of undesired mutations introduced by the polymerase. Second, a selection must be employed in order to reduce the number of non-mutated parental molecules persisting in the reaction. Third, an extended-length PCR method is preferred in order to allow the use of a single PCR primer set. And fourth, because of the non-template-dependent terminal extension activity of some thermostable polymerases it is often necessary to incorporate an end-polishing step into the procedure prior to blunt-end ligation of the PCR-generated mutant product.

Mutations may also be introduced by directed evolution processes, such as phage-assisted continuous evolution (PACE) or phage-assisted noncontinuous evolution (PANCE). The term “phage-assisted continuous evolution (PACE),” as used herein, refers to continuous evolution that employs phage as viral vectors. The general concept of PACE technology has been described, for example, in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Application, U.S. Pat. No. 9,023,594, issued May 5, 2015, International PCT Application, PCT/US2015/012022, filed Jan. 20, 2015, published as WO 2015/134121 on Sep. 11, 2015, and International PCT Application, PCT/US2016/027795, filed Apr. 15, 2016, published as WO 2016/168631 on Oct. 20, 2016, the entire contents of each of which are incorporated herein by reference. Variant Cas9s may also be obtain by phage-assisted non-continuous evolution (PANCE),” which as used herein, refers to non-continuous evolution that employs phage as viral vectors. PANCE is a simplified technique for rapid in vivo directed evolution using serial flask transfers of evolving ‘selection phage’ (SP), which contain a gene of interest to be evolved, across fresh E. coli host cells, thereby allowing genes inside the host E. coli to be held constant while genes contained in the SP continuously evolve. Serial flask transfers have long served as a widely-accessible approach for laboratory evolution of microbes, and, more recently, analogous approaches have been developed for bacteriophage evolution. The PANCE system features lower stringency than the PACE system.

Any of the references noted above which relate to Cas9 or Cas9 equivalents are hereby incorporated by reference in their entireties, if not already stated so.

### III. Evolved DddA Containing Base Editors

Provided herein are evolved-DddA containing base editors.

The present disclosure provides evolved DddA proteins or fragments thereof and base editor fusion proteins comprising same. The disclosure also provide vectors and nucleic acid molecules encoding said base editor fusion proteins, kits, and methods of modifying double-stranded DNA (e.g., mtDNA) using genome editing strategies that comprise the use of a programmable DNA binding protein (“pDNAbp”) (e.g., a mitoTALE, mitoZFP, or a CRISPR/Cas9) and a double-stranded DNA deaminase (“DddA”) to precisely install nucleotide changes and/or correct pathogenic mutations in mtDNA, rather than destroying the mtDNA with double-strand breaks (DSBs). In various embodiments, these polypeptides may be combined as fusion proteins referred to as “evolved-DddA containing base editors.” In various embodiments, that base editor fusion proteins may be provided as separate components, i.e., not as a fusion protein, but rather as separate pDNAbp and DddA domains which associate in the cell to target the desired edit site.

Also provided herein are base editor fusion proteins, vectors and nucleic acid molecule encoding base editor fusion proteins, kits, and methods of modifying any double-stranded DNA (e.g., genomic DNA) using genome editing strategies that comprise the use of a programmable DNA binding protein (“pDNAbp”) (e.g., a mitoTALE, mitoZFP, or a CRISPR/Cas9) and a double-stranded DNA deaminase (“DddA”) to precisely install nucleotide changes and/or correct pathogenic mutations in double-stranded DNA, rather than destroying the DNA with double-strand breaks (DSBs). In various embodiments, that base editor fusion proteins may be provided as separate components, i.e., not as a fusion protein, but rather as separate pDNAbp and DddA domains which associate in the cell to target the desired edit site.

The present disclosure provides evolved-DddA containing base editors, pDNAbp polypeptides, DddA polypeptides, nucleic acid molecules encoding the pDNAbp polypeptides, DddA polypeptides, and fusion proteins described herein, expression vectors comprising the nucleic acid molecules, cells comprising the nucleic acid molecules, expression vectors, and/or pDNAbp polypeptides, DddA polypeptides, or fusion proteins, pharmaceutical compositions comprising the polypeptides, fusion proteins, nucleic acid molecules, vectors, or the cells described herein, and kits comprising the polypeptides, fusion proteins, nucleic acid molecules, vectors, or the cells described herein for modifying mtDNA by base editing.

In some embodiments, the Evolved DddA-containing base editors comprise a pDNAbp (e.g., a mitoTALE, mitoZFP, or a CRISPR/Cas) and a DddAs (or inactive fragment there). In other embodiments, the Evolved DddA-containing base editors comprise separately expressed pDNAbps and DddAs, which may be co-localized at a desired target site through the use of split-intein sequences, RNA-protein recruitment systems, or other elements that facilitate the co-localization of separately expressed elements to a target site. In various other embodiments, the fusion proteins and/or the separately expressed pDNAbps and DddAs become translocated into the mitochondria. To effect translocation, the fusion proteins and/or the separately expressed pDNAbps and DddAs can comprise one or more mitochondrial targeting sequences (MTS).

In still other embodiments, the Evolved DddA-containing base editors comprise a Evolved DddA domain which has been inactivated. In one embodiment, this inactivation can be achieved by engineering a whole DddA polypeptide into two or more fragments, each alone which is inactive and non-toxic to a cell. When the DddA inactive fragments become co-localization in the cell, e.g., inside the mitochondria, the fragments reconstitute the deaminase activity. The co-localization of the DddA fragments can be effectuated by fusing each DddA fragment to a separate pDNAbp that binds on either one side or the other of a target deamination site. For example, the embodiments depicted in FIGS. 1A-1F show that splitting the DddA at a split site into two inactive DddA fragments (e.g., “DddA half^{A}” and “DddA half^{B}”) result in a non-toxic form of DddA. FIG. 1B shows that each of the inactive DddA fragments may be separately expressed as a fusion protein with a pDNAbp which binds to separate target sites on either side of a target deamination site. In FIG. 1B, these target sites are represented by “target site A” and “target site B”. By binding the pDNAbp domain of each of the fusion protein to their respective sites, the inactive DddA fragments become co-localized at the desired deamination site, thereby restoring the deaminase activity of the original DddA enzyme. FIGS. 1C, 1D, and 1E show this arrangement in the context of a mitoTALE, mitoZFP, and a Cas9/sgRNA complex as the pDNAbp domain of the evolved-DddA containing base editors.

In certain embodiments, the reconstituted activity of the co-localized two or more fragments can comprise at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or at least 99.9% of the deaminase activity of a wildtype DddA.

In terms of the spacing between the target site A and target site B from the site of deamination, any suitable spacing may be used, and which may be further dependent on the length of the linkers (if present) between the pDNAbp and the DddA domains, as well as the properties of the DddA domains. If the target nucleobase site (C on the deamination strand or a G:C nucleobase pair if referring to both strands) is assigned an arbitrary value of 0, then 3′-most position of target site A, in various embodiments, may be spaced at least 1 nucleotide upstream of the target G:C nucleobase pair, or at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides upstream of the G:C nucleobase pair (or otherwise the target site of deamination). Likewise, the 3′-most position of target site B (i.e., which is on the opposite strand in this instance), may be spaced at least 1 nucleotide upstream of the target G:C nucleobase pair, or at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides upstream of the G:C nucleobase pair (or otherwise the target site of deamination).

Looking at FIG. 1B-1F as a point of reference, it is also contemplated herein that target site A and target site B may be on the same strand of DNA. That is, the inactive DddA fragments may become co-localized at the desired site of deamination by using a pair of evolved-DddA containing base editor fusion proteins having pDNAbp components (e.g., mitoTALEs, mitoZFP, Cas9 domains) that both bind to target sites A and B on the same strand. In the case where a pair of Evolved DddA-containing base editors bind to the same strand of DNA at separate target sites, the strand of DNA containing the target sites can be the same strand at the site of deamination, or the strand can be the opposite strand. So long as the inactive DddA fragments become co-localized at the intended site of deamination, the pair of base editor fusion proteins may bind to target sites on the same strands or opposite strands, and when binding to the same strand, the target sites can be the same or the opposite strand as the strand having the site of deamination.

In certain embodiments, the DddA can be separated into two fragments by dividing the DddA at a split site. A “split site” refers to a position between two adjacent amino acids (in a wildtype DddA amino acid sequence) that marks a point of division of a DddA. In certain embodiments, the DddA can have a least one split site, such that once divided at that split site, the DddA forms an N-terminal fragment and a C-terminal fragment. The N-terminal and C-terminal fragments can be the same or difference sizes (or lengths), wherein the size and/or polypeptide length depends on the location or position of the split site. As used herein, reference to a “fragment” of DddA (or any other polypeptide) can be referred equivalently as a “portion.” Thus, a DddA which is divided at a split site can form an N-terminal portion and a C-terminal portion. Preferably, the N-terminal fragment (or portion) and the C-terminal fragment (or portion) or DddA do not have a deaminase activity.

In various embodiments, a DddA may be split into two or more inactive fragments by directly cleaving the DddA at one or more split sites. Direct cleaving can be carried out by a protease (e.g., trypsin) or other enzyme or chemical reagent. In certain embodiments, such chemical cleavage reactions can be designed to be site-selective (e.g., Elashal and Raj, “Site-selective chemical cleavage of peptide bonds,” Chemical Communications, 2016, Vol. 52, pages 6304-6307, the contents of which are incorporated herein by reference.) In other embodiments, chemical cleavage reactions can be designed to be non-selective and/or occur in a random fashion.

In other embodiments, the two or more inactive DddA fragments can be engineered as separately expressed polypeptides. For instance, for a DddA having one split site, the N-terminal DddA fragment could be engineered from a first nucleotide sequence that encodes the N-terminal DddA fragment (which extends from the N-terminus of the DddA up to and including the residue on the amino-terminal side of the split site). In such an example, the C-terminal DddA fragment could be engineered from a second nucleotide sequence that encodes the C-terminal DddA fragment (which extends from the carboxy-terminus of the split site up to including the natural C-terminus of the DddA protein). The first and second nucleotide sequences could be on the same or different nucleotide molecules (e.g., the same or different expression vectors).

In various embodiments, that N-terminal portion of the DddA may be referred to as “DddA-N half” and the C-terminal portion of the DddA may be referred to as the “DddA-C half.” Reference to the term “half” does not connote the requirement that the DddA-N and DddA-C portions are identically half of the size and/or sequence length of a complete DddA, or that the split site is required to be at the mid point of the complete DddA polypeptide. To the contrary, and as noted above, the split site can be between any pair of residues in the DddA polypeptide, thereby giving rise to half portions which are unequal in size and/or sequence length. In certain embodiments, the split site is within a loop region of the DddA.

Accordingly, in one aspect, the disclosure relates to a pair of fusion proteins useful for making modifications to the sequence of mitochondrial DNA (e.g., mtDNA). The pair of fusion proteins, in some embodiments, can comprise a first fusion protein comprising a first pDNAbp (e.g., a mitoTALE, mitoZFP, or a CRISPR/Cas9) and a first portion or fragment of a DddA, and a second fusion protein comprising a second pDNAbp (e.g., mitoTALE, mitoZFP, or a CRISPR/Cas9) and a second portion or fragment of a DddA, such that the first and the second portions of the DddA reconstitute a DddA upon co-localization in a cell and/or mitochondria. In certain embodiments, that first portion of the DddA is an N-terminal fragment of a DddA and the second portion of the DddA is C-terminal fragment of a DddA. In other embodiments, the first portion of the DddA is a C-terminal fragment of a DddA and the second portion of the DddA is an N-terminal fragment of a DddA. In this aspect, the structure of the pair of fusion proteins can be, for example:
[pDNAbp]-[DddA half^{A}] and [pDNAbp]-[DddA half^{B}];
[DddA-half^{A}]-[pDNAbp] and [DddA-half^{B}]-[pDNAbp];
[pDNAbp]-[DddA half^{A}] and [DddA-half^{B}]-[pDNAbp]; or
[DddA-half^{A}]-[pDNAbp] and [pDNAbp]-[DddA half^{B}], wherein “A” or “B” can be the N-terminal or C-terminal half of DddA.

In another aspect, the disclosure relates to a pair of fusion proteins useful for making modifications to the sequence of mitochondrial DNA (e.g., mtDNA). The pair of fusion proteins can comprise a first fusion protein comprising a first mitoTALE and a first portion or fragment of a DddA, and a second fusion protein comprising a second mitoTALE and a second portion or fragment of a DddA, such that the first and the second portions of the DddA, upon co-localization in a cell and/or mitochondria, are reconstituted an active DddA. In certain embodiments, that first portion of the DddA is an N-terminal fragment of a DddA and the second portion of the DddA is C-terminal fragment of a DddA. In other embodiments, the first portion of the DddA is a C-terminal fragment of a DddA and the second portion of the DddA is an N-terminal fragment of a DddA. In this aspect, the structure of the pair of fusion proteins can be, for example:
[mitoTALE]-[DddA half^{A}] and [mitoTALE]-[DddA half^{B}];
[DddA-half^{A}]-[pDNAbp] and [DddA-half^{B}]-[mitoTALE];
[mitoTALE]-[DddA half^{A}] and [DddA-half^{B}]-[mitoTALE]; or
[DddA-half^{A}]-[mitoTALE] and [mitoTALE]-[DddA half^{B}], wherein “A” or “B” can be the N-terminal or C-terminal half of DddA.

In yet another aspect, the disclosure relates to a pair of fusion proteins useful for making modifications to the sequence of mitochondrial DNA (e.g., mtDNA). The pair of fusion proteins can comprise a first fusion protein comprising a first mitoZFP and a first portion or fragment of a DddA, and a second fusion protein comprising a second mitoZFP and a second portion or fragment of a DddA, such that the first and the second portions of the DddA, upon co-localization in a cell and/or mitochondria, are reconstituted an active DddA. In certain embodiments, that first portion of the DddA is an N-terminal fragment of a DddA and the second portion of the DddA is C-terminal fragment of a DddA. In other embodiments, the first portion of the DddA is a C-terminal fragment of a DddA and the second portion of the DddA is an N-terminal fragment of a DddA. In this aspect, the structure of the pair of fusion proteins can be, for example:
[mitoZFP]-[DddA half^{A}] and [mitoZFP]-[DddA half^{B}];
[DddA-half^{A}]-[pDNAbp] and [DddA-half^{B}]-[mitoZFP];
[mitoZFP]-[DddA half^{A}] and [DddA-half^{B}]-[mitoZFP]; or
[DddA-half^{A}]-[mitoZFP] and [mitoZFP]-[DddA half^{B}], wherein “A” or “B” can be the N-terminal or C-terminal half of DddA.

In yet another aspect, the disclosure relates to a pair of fusion proteins useful for making modifications to the sequence of mitochondrial DNA (e.g., mtDNA). The pair of fusion proteins can comprise a first fusion protein comprising a first Cas9 and a first portion or fragment of a DddA, and a second fusion protein comprising a second Cas9 and a second portion or fragment of a DddA, such that the first and the second portions of the DddA, upon co-localization in a cell and/or mitochondria, are reconstituted an active DddA. In certain embodiments, that first portion of the DddA is an N-terminal fragment of a DddA (i.e., “DddA half^{A}” as shown in FIGS. 1A-1E) and the second portion of the DddA is C-terminal fragment of a DddA (i.e., “DddA half^{B}” as shown in FIGS. 1A-1E). In other embodiments, the first portion of the DddA is an C-terminal fragment of a DddA and the second portion of the DddA is an N-terminal fragment of a DddA. In this aspect, the structure of the pair of fusion proteins can be, for example:
[Cas9]-[DddA half^{A}] and [Cas9]-[DddA half^{B}];
[DddA-half^{A}]-[Cas9] and [DddA-half^{B}]-[Cas9];
[Cas9]-[DddA half^{A}] and [DddA-half^{B}]-[Cas9]; or
[DddA-half^{A}]-[Cas9] and [Cas9]-[DddA half^{B}], wherein “A” or “B” can be the N-terminal or C-terminal half of DddA.

In each instance above of “]-[” can be in reference to a linker sequence.

In addition, the fusion proteins may have any suitable architecture, include any those depicted in FIG. 1F.

In some embodiments, a first fusion protein comprises, a first mitochondrial transcription activator-like effector (mitoTALE) domain and a first portion of a DNA deaminase effector (DddA).

In some embodiments, the first portion of the DddA comprises an N-terminal truncated DddA. In some embodiments, the first mitoTALE is configured to bind a first nucleic acid sequence proximal to a target nucleotide. In some embodiments, the first portion of a DddA is linked to the remainder of the first fusion protein by the C-terminus of the first portion of a DddA.

In one aspect, the present disclosure provides mitochondrial DNA editor fusion proteins for use in editing mitochondrial DNA. As used herein, these mitochondrial DNA editor fusion proteins may be referred to as “mtDNA editors” or “mtDNA editing systems.”

In various embodiments, the mtDNA editors described herein comprise (1) a programmable DNA binding protein (“pDNAbp”) (e.g., a mitoTALE domain, mitoZFP domain, or a CRISPR/Cas9 domain) and a double-stranded DNA deaminase domain, which is capable of carrying out a deamination of a nucleobase at a target site associated with the binding site of the programmable DNA binding protein (pDNAbp).

In some embodiments, the double-stranded DNA deaminase is split into two inactive half portions, with each half portion being fused to a programmable DNA binding protein that binds to a nucleotide sequence either upstream or downstream of a target edit site, and wherein once in the mitochondria, the two half portions (i.e., the N-terminal half and the C-terminal half) reassociate at the target edit site by the co-localization of the programmable DNA binding proteins to binding sites upstream and downstream of the target edit site to be acted on by the DNA deaminase. The reassociation of the two half portions of the double-stranded DNA deaminase restores the deaminase activity at the target edit site. In other embodiments, the double-stranded DNA deaminase can initially be set in an inactive state which can be induced when in the mitochondria. The double-stranded DNA deaminase is preferably delivered initially in an inactive form in order to avoid toxicity inherent with the protein. Any means to regulate the toxic properties of the double-stranded DNA deaminase until such time as the activity is desired to be activated (e.g., in the mitochondria) is contemplated.

The Evolved DddA-containing base editors described herein contemplate fusion proteins comprising a mitoTALE and a Evolved DddA domain or fragment or portion thereof (e.g., an N-terminal or C-terminal fragment or portion of a DddA), and optionally the joining of the two by a linker. The application contemplates any suitable mitoTALE and a Evolved DddA domain to be combined in a single fusion protein. Examples of mitoTALEs and DddA domains are each defined herein.

In some embodiments, a first fusion protein comprises a first portion of a DddA fused (e.g., attached) to a first mitoTALE. In some embodiments, a second fusion protein comprises a second portion of a DddA fused (e.g., attached) to a second mitoTALE. In some embodiments, the first fusion protein comprises a first portion of a DddA linked to the remainder of the first fusion protein by the C-terminus of the first portion of a DddA. In some embodiments, a second fusion protein comprises a second portion of a DddA linked to the remainder of the second fusion protein by the C-terminus of the second portion of a DddA.

In some embodiments, the first fusion protein comprises a first mitoTALE to bind a target nucleic acid sequence proximal (as defined herein above) to the target nucleotide. In some embodiments, the second fusion protein comprises a mitoTALE to bind a target nucleic acid sequence proximal to the nucleotide complementary to the target nucleotide. In some embodiments, the first and second mitoTALEs are configured to bind proximally to the same target nucleotide (or nucleotide complementary thereto, as described herein above). In some embodiments, the first and second fusion proteins comprise mitoTALEs configured to bind first and second target nucleic acid sequences such that the first and second portions of DddA can dimerize (i.e., re-assemble) at or near the target nucleotide, such that re-assembled first and second portions of a DddA regain, at least partially, the native activity (e.g., deamination) of a full-length DddA. In some embodiments, the first and second fusion proteins comprise mitoTALEs configured to bind first and second target nucleic acid sequences such that that the first and second portions of a DddA can dimerize (i.e., re-assemble) at or near the target nucleotide, such that the target nucleotide is affected by activity of a re-assembled first and second portions of a DddA. Any suitable architecture of the fusion proteins comprising mitoTALEs are contemplated, and shows in FIG. 1F.

The Evolved DddA-containing base editors described herein also contemplate fusion proteins comprising a mitoZF and a Evolved DddA domain or fragment or portion thereof (e.g., an N-terminal or C-terminal fragment or portion of a DddA), and optionally the joining of the two by a linker. The application contemplates any suitable mitoZF and a Evolved DddA domain to be combined in a single fusion protein. Examples of mitoZFs and DddA domains are each defined herein.

In some embodiments, a first fusion protein comprises a first portion of a DddA fused (e.g., attached) to a first mitoZF. In some embodiments, a second fusion protein comprises a second portion of a DddA fused (e.g., attached) to a second mitoZF. In some embodiments, the first fusion protein comprises a first portion of a DddA linked to the remainder of the first fusion protein by the C-terminus of the first portion of a DddA. In some embodiments, a second fusion protein comprises a second portion of a DddA linked to the remainder of the second fusion protein by the C-terminus of the second portion of a DddA.

In some embodiments, the first fusion protein comprises a first mitoZF to bind a target nucleic acid sequence proximal (as defined herein above) to the target nucleotide. In some embodiments, the second fusion protein comprises a mitoZF to bind a target nucleic acid sequence proximal to the nucleotide complementary to the target nucleotide. In some embodiments, the first and second mitoZFs are configured to bind proximally to the same target nucleotide (or nucleotide complementary thereto, as described herein above). In some embodiments, the first and second fusion proteins comprise mitoZFs configured to bind first and second target nucleic acid sequences such that the first and second portions of DddA can dimerize (i.e., re-assemble) at or near the target nucleotide, such that re-assembled first and second portions of a DddA regain, at least partially, the native activity (e.g., deamination) of a full-length DddA. In some embodiments, the first and second fusion proteins comprise mitoTALEs configured to bind first and second target nucleic acid sequences such that that the first and second portions of a DddA can dimerize (i.e., re-assemble) at or near the target nucleotide, such that the target nucleotide is affected by activity of a re-assembled first and second portions of a DddA. Any suitable architecture of the fusion proteins comprising mitoZFs are contemplated, and shows in FIG. 1F.

In some embodiments, the first fusion protein comprises the amino acid sequence of any one of SEQ ID NOs.: 5, 10-12, 147, 149, 151, 154, 156, 161, 165, 167, and 170-173. In some embodiments, the first fusion protein comprises an amino acid sequence with 75% or greater percent identity (e.g., 80% or greater, 85% or greater, 90% or greater, 95% or greater, 96% or greater, 97% or greater, 98% or greater, 99% or greater, 99.5% or greater, 99.9% or greater percent identity) any one of SEQ ID NOs.: 5, 10-12, 147, 149, 151, 154, 156, 161, 165, 167, and 170-173. In some embodiments, the second fusion protein comprises the amino acid sequence of any one of SEQ ID NOs.: 5, 10-12, 147, 149, 151, 154, 156, 161, 165, 167, and 170-173. In some embodiments, the second fusion protein comprises an amino acid sequence with 75% or greater percent identity (e.g., 80% or greater, 85% or greater, 90% or greater, 95% or greater, 96% or greater, 97% or greater, 98% or greater, 99% or greater, 99.5% or greater, 99.9% or greater percent identity) to any one of SEQ ID NOs.: 5, 10-12, 147, 149, 151, 154, 156, 161, 165, 167, and 170-173.

In some embodiments, the first and second fusion protein form pairs which result from the targeting of a similar target nucleotide, or which first and second portion of a DddA form a pair of portions which can re-assemble (e.g., dimerize) to form a protein with, at least partially, the activity of a full-length DddA (e.g., deamination). In some embodiments, the pair of fusion proteins comprise a first fusion protein comprising the first fusion protein of any one of and a second fusion protein comprising the second fusion protein wherein the first mitoTALE of the first fusion protein is configured to bind a first nucleic acid sequence proximal to a target nucleotide and the second mitoTALE of the second fusion protein is configured to bind a second nucleic acid sequence proximal to a nucleotide opposite the target nucleotide. In some embodiments, the first nucleic acid sequence is upstream of the target nucleotide and the second nucleic acid sequence is upstream of a nucleic acid of the complementary nucleotide of the target nucleotide. In some embodiments, the re-assembly (i.e., dimerization) of the first and second fusion proteins facilitate deamination of the target nucleotide.

### Base Editors Comprising MitoTALES

The Evolved DddA-containing base editors described herein contemplate fusion proteins comprising a mitoTALE and an evolved DddA domain or fragment or portion thereof (e.g., an N-terminal or C-terminal fragment or portion of a DddA), and optionally the joining of the two by a linker. The application contemplates any suitable mitoTALE and a Evolved DddA domain to be combined in a single fusion protein. Examples of mitoTALEs and DddA domains are each defined herein.

In some embodiments, the Evolved DddA-containing base editors comprise DddA domains which are DdCBE, i.e., DddA which deaminates a C. Examples of general architecture of Evolved DddA-containing base editors comprising DdCBEs and mitoTALEs and their amino acid and nucleotide sequences are represented by SEQ ID NOs: 11-15 and 144-170.

All right-side halves of DdCBEs have the general architecture of (from N- to C-terminus): COX8A MTS-3×FLAG-mitoTALE-2aa linker-DddAₜₒₓ half-4aa linker-1×-UGI-ATP5B 3′UTR

All left-side halves of DdCBEs have the general architecture of (from N- to C-terminus): SOD2 MTS-3×HA-mitoTALE-2aa linker-DddAₜₒₓ half-4aa linker-1×-UGI-SOD2 3′UTR

Other exemplary Evolved DddA-containing base editors may comprise DdCBE/mitoTALE fusion proteins represented by SEQ ID NOs: 5, 10-12, 147, 149, 151, 154, 156, 161, 165, 167, and 170-173.:

All right-side halves of DdCBEs have the general architecture of (from N- to C-terminus): COX8A MTS-3×FLAG-mitoTALE-2aa linker-DddAtox half-4aa linker-1×-UGI-ATP5B 3′UTR

All left-side halves of DdCBEs have the general architecture of (from N- to C-terminus): SOD2 MTS-3×HA-mitoTALE-2aa linker-DddAtox half-4aa linker-1×-UGI-SOD2 3′UTR

ND6-DdCBE: Left mitoTALE-G1397-DddAtox-N-1x-UGI (Note: Terminal
NG RVD recognizes a mismatched T instead of a G in the reference
genome)
(SEQ ID NO: 5)
MALSRAVCGTSRQLAPVLGYLGSRQKHSLPDYPYDVPDYAGYPYDVPDYAGYPYDVPDYAMDIADL
RTLGYSQQQQEKIKPKVRSTVAQHHEALVGHGFTHAHIVALSQHPAALGTVAVKYQDMIAALPEATH
EAIVGVGKQWSGARALEALLTVAGELRGPPLQLDTGQLLKIAKRGGVTAVEAVHAWRNALTGAPLN
LTPQQVVAIASNNGGKQALETVQRLLPVLCQAHGLTPEQVVAIASNIGGKQALETVQALLPVLCQAH
GLTPEQVVAIASHDGGKQALETVQRLLPVLCQAHGLTPEQVVAIASHDGGKQALETVQRLLPVLCQA
HGLTPEQVVAIASHDGGKQALETVQRLLPVLCQAHGLTPEQVVAIASHDGGKQALETVQRLLPVLCQ
AHGLTPEQVVAIASHDGGKQALETVQRLLPVLCQAHGLTPEQVVAIASNIGGKQALETVQALLPVLC
QAHGLTPQQVVAIASNGGGKQALETVQRLLPVLCQAHGLTPQQVVAIASNGGGRPALESIVAQLSRP
DPALAALTNDHLVALACLGGRPALDAVKKGLGGSGSYALGPYQISAPQLPAYNGQTVGTFYYVNDA
GGLESKVFSSGGPTPYPNYANAGHVEGQSALFMRDNGISEGLVFHNNPEGTCGFCVNMTETLLPENA
KMTVVPPEGSGGSTNLSDIIEKETGKQLVIQESILMLPEEVEEVIGNKPESDILVHTAYDESTDENVMLL
TSDAPEYKPWALVIQDSNGENKIKML**
ND6-DdCBE: Right mitoTALE-G1397-DddAtox-N-1x-UGI (Note: Terminal
NG RVD recognizes a mismatched T instead of a G in the reference
genome. The NTD was also engineered to be permissive for A, T, C
and G nucleotides at the NO position)
(SEQ ID NO: 10)
MASVLTPLLLRGLTGSARRLPVPRAKIHSLDYKDHDGDYKDHDIDYKDDDDKMDIADLRTLGYSQQ
QQEKIKPKVRSTVAQHHEALVGHGFTHAHIVALSQHPAALGTVAVKYQDMIAALPEATHEAIVGVGK
RGAGARALEALLTVAGELRGPPLQLDTGQLLKIAKRGGVTAVEAVHAWRNALTGAPLNLTPQQVVA
IASNNGGKQALETVQRLLPVLCQAHGLTPEQVVAIASNIGGKQALETVQALLPVLCQAHGLTPQQVV
AIASNGGGKQALETVQRLLPVLCQAHGLTPQQVVAIASNNGGKQALETVQRLLPVLCQAHGLTPQQV
VAIASNNGGKQALETVQRLLPVLCQAHGLTPEQVVAIASHDGGKQALETVQRLLPVLCQAHGLTPQQ
VVAIASNGGGKQALETVQRLLPVLCQAHGLTPEQVVAIASNIGGKQALETVQALLPVLCQAHGLTPQ
QVVAIASNGGGKQALETVQRLLPVLCQAHGLTPQQVVAIASNGGGKQALETVQRLLPVLCQAHGLTP
QQVVAIASNGGGRPALESIVAQLSRPDPALAALTNDHLVALACLGGRPALDAVKKGLGGSAIPVKRG
ATGETKVFTGNSNSPKSPTKGGCSGGSTNLSDIIEKETGKQLVIQESILMLPEEVEEVIGNKPESDILVHT
AYDESTDENVMLLTSDAPEYKPWALVIQDSNGENKIKML**
ND1-DdCBE Right mitoTALE repeat
(SEQ ID NO: 147)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASNNGGKQALETVQALLPVLCQAHGLTPQQVVAIASNIGGKQALETVQRLLPVL
CQAHGLTPQQVVAIASNNGGKQALETVQRLLPVLCQAHGLTPEQVVAIASNGGGKQALETVQALLPV
LCQAHGLTPEQVVAIASNGGGKQALETVQALLPVLCQAHGLTPEQVVAIASNGGGKQALETVQALLP
VLCQAHGLTPEQVVAIASNNGGKQALETVQRLLPVLCQAHGLTPEQVVAIASNIGGKQALETVQALL
PVLCQAHGLTPEQVVAIASNGGGKQALETVQRLLPVLCQAHGLTPEQVVAIASNNGGKQALETVQAL
LPVLCQAHGLTPEQVVAIASHDGGKQALETVQALLPVLCQAHGLTPEQVVAIASNGGGKQALETVQA
LLPVLCQAHGLTPEQVVAIASHDGGKQALETVQRLLPVLCQAHGLTPQQVVAIASNIGGKQALETVQ
RLLPVLCQAHGLTPEQVVAIASHDGGKQALETVQALLPVLCQAHGLTPQQVVAIASHDGGKQALETV
QRLLPVLCQAHGLTPQQVVAIASHDGGKQALETVQRLLPVLCQAHGLTPQQVVAIASNGGGRPALESI
VAQLSRPDPALAALTNDHLVALACLGGRPALDAVKKGLG
ND1-DdCBE Left mitoTALE repeat
(SEQ ID NO: 149)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASHDGGKQALETVQALLPVLCQAHGLTPQQVVAIASNGGGKQALETVQRLLPV
LCQAHGLTPQQVVAIASNIGGKQALETVQRLLPVLCQAHGLTPEQVVAIASNNGGKQALETVQALLP
VLCQAHGLTPEQVVAIASHDGGKQALETVQALLPVLCQAHGLTPEQVVAIASHDGGKQALETVQALL
PVLCQAHGLTPEQVVAIASNGGGKQALETVQRLLPVLCQAHGLTPEQVVAIASNIGGKQALETVQAL
LPVLCQAHGLTPEQVVAIASNNGGKQALETVQRLLPVLCQAHGLTPEQVVAIASHDGGKQALETVQA
LLPVLCQAHGLTPEQVVAIASHDGGKQALETVQALLPVLCQAHGLTPEQVVAIASNNGGKQALETVQ
ALLPVLCQAHGLTPEQVVAIASNGGGKQALETVQRLLPVLCQAHGLTPQQVVAIASNGGGKQALETV
QRLLPVLCQAHGLTPQQVVAIASNGGGRPALESIVAQLSRPDPALAALTNDHLVALACLGGRPALDA
VKKGLG
ND2-DdCBE Right mitoTALE repeat
(SEQ ID NO: 151)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASNIGGKQALETVQALLPVLCQAHGLTPQQVVAIASNNGGKQALETVQRLLPVL
CQAHGLTPQQVVAIASNIGGKQALETVQRLLPVLCQAHGLTPEQVVAIASNIGGKQALETVQALLPVL
CQAHGLTPEQVVAIASHDGGKQALETVQALLPVLCQAHGLTPEQVVAIASNGGGKQALETVQALLPV
LCQAHGLTPEQVVAIASNNGGKQALETVQRLLPVLCQAHGLTPEQVVAIASHDGGKQALETVQALLP
VLCQAHGLTPEQVVAIASNGGGKQALETVQRLLPVLCQAHGLTPEQVVAIASNIGGKQALETVQALL
PVLCQAHGLTPEQVVAIASNGGGKQALETVQALLPVLCQAHGLTPEQVVAIASNGGGKQALETVQAL
LPVLCQAHGLTPEQVVAIASNIGGKQALETVQRLLPVLCQAHGLTPQQVVAIASNGGGKQALETVQR
LLPVLCQAHGLTPQQVVAIASNGGGRPALESIVAQLSRPDPALAALTNDHLVALACLGGRPALDAVK
KGLG
ND2-DdCBE Left mitoTALE repeat
(SEQ ID NO: 171)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASHDGGKQALETVQALLPVLCQAHGLTPQQVVAIASNGGGKQALETVQRLLPV
LCQAHGLTPQQVVAIASNGGGKQALETVQRLLPVLCQAHGLTPEQVVAIASNIGGKQALETVQALLP
VLCQAHGLTPEQVVAIASNNGGKQALETVQALLPVLCQAHGLTPEQVVAIASHDGGKQALETVQALL
PVLCQAHGLTPEQVVAIASNIGGKQALETVQRLLPVLCQAHGLTPEQVVAIASNGGGKQALETVQAL
LPVLCQAHGLTPEQVVAIASNIGGKQALETVQRLLPVLCQAHGLTPEQVVAIASHDGGKQALETVQA
LLPVLCQAHGLTPEQVVAIASNGGGKQALETVQALLPVLCQAHGLTPEQVVAIASHDGGKQALETVQ
ALLPVLCQAHGLTPEQVVAIASHDGGKQALETVQRLLPVLCQAHGLTPQQVVAIASNGGGKQALETV
QRLLPVLCQAHGLTPEQVVAIASHDGGKQALETVQALLPVLCQAHGLTPEQVVAIASNIGGKQALET
VQALLPVLCQAHGLTPEQVVAIASNIGGKQALETVQALLPVLCQAHGLTPQQVVAIASNGGGRPALES
IVAQLSRPDPALAALTNDHLVALACLGGRPALDAVKKGLG
ND4-DdCBE Right mitoTALE repeat
(SEQ ID NO: 154)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASHDGGKQALETVQALLPVLCQAHGLTPQQVVAIASHDGGKQALETVQRLLPV
LCQAHGLTPQQVVAIASNGGGKQALETVQRLLPVLCQAHGLTPEQVVAIASNNGGKQALETVQALLP
VLCQAHGLTPEQVVAIASNGGGKQALETVQALLPVLCQAHGLTPEQVVAIASNIGGKQALETVQALL
PVLCQAHGLTPEQVVAIASNIGGKQALETVQRLLPVLCQAHGLTPEQVVAIASNNGGKQALETVQAL
LPVLCQAHGLTPEQVVAIASNGGGKQALETVQRLLPVLCQAHGLTPEQVVAIASNIGGKQALETVQR
LLPVLCQAHGLTPEQVVAIASNNGGKQALETVQALLPVLCQAHGLTPEQVVAIASNNGGKQALETVQ
ALLPVLCQAHGLTPEQVVAIASNIGGKQALETVQALLPVLCQAHGLTPEQVVAIASNNGGKQALETV
QRLLPVLCQAHGLTPQQVVAIASNIGGKQALETVQRLLPVLCQAHGLTPQQVVAIASNNGGKQALET
VQRLLPVLCQAHGLTPQQVVAIASNGGGRPALESIVAQLSRPDPALAALTNDHLVALACLGGRPALD
AVKKGLG
ND4-DdCBE Left mitoTALE repeat
(SEQ ID NO: 156)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASNNGGKQALETVQALLPVLCQAHGLTPQQVVAIASHDGGKQALETVQRLLPV
LCQAHGLTPQQVVAIASNGGGKQALETVQRLLPVLCQAHGLTPEQVVAIASNIGGKQALETVQALLP
VLCQAHGLTPEQVVAIASNNGGKQALETVQALLPVLCQAHGLTPEQVVAIASNGGGKQALETVQALL
PVLCQAHGLTPEQVVAIASNIGGKQALETVQRLLPVLCQAHGLTPEQVVAIASNIGGKQALETVQALL
PVLCQAHGLTPEQVVAIASHDGGKQALETVQRLLPVLCQAHGLTPEQVVAIASHDGGKQALETVQAL
LPVLCQAHGLTPEQVVAIASNIGGKQALETVQALLPVLCQAHGLTPEQVVAIASHDGGKQALETVQA
LLPVLCQAHGLTPEQVVAIASNNGGKQALETVQRLLPVLCQAHGLTPQQVVAIASNGGGKQALETVQ
RLLPVLCQAHGLTPEQVVAIASNGGGKQALETVQALLPVLCQAHGLTPEQVVAIASHDGGKQALETV
QALLPVLCQAHGLTPQQVVAIASNGGGRPALESIVAQLSRPDPALAALTNDHLVALACLGGRPALDA
VKKGLG
ND5.1-DdCBE Right mitoTALE repeat
(SEQ ID NO: 11)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPQQVVAIASHDGGKQALETVQRLLPVLCQAHGLTPEQVVAIASNGGGKQALETVQRLLPV
LCQAHGLTPQQVVAIASNGGGKQALETVQRLLPVLCQAHGLTPEQVVAIASNGGGKQALETVQALLP
VLCQAHGLTPQQVVAIASNNGGKQALETVQRLLPVLCQAHGLTPQQVVAIASNNGGKQALETVQRL
LPVLCQAHGLTPEQVVAIASNIGGKQALETVQRLLPVLCQAHGLTPQQVVAIASNNGGKQALETVQR
LLPVLCQAHGLTPQQVVAIASNGGGKQALETVQRLLPVLCQAHGLTPEQVVAIASNIGGKQALETVQ
RLLPVLCQAHGLTPEQVVAIASNNGGKQALETVQRLLPVLCQAHGLTPQQVVAIASNIGGKQALETV
QRLLPVLCQAHGLTPQQVVAIASNIGGRPALESIVAQLSRPDPALAALTNDHLVALACLGGRPALDAV
KKGLG
ND5.1-DdCBE Left mitoTALE repeat
(SEQ ID NO: 172)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASNIGGKQALETVQALLPVLCQAHGLTPQQVVAIASNNGGKQALETVQRLLPVL
CQAHGLTPQQVVAIASHDGGKQALETVQRLLPVLCQAHGLTPEQVVAIASNIGGKQALETVQALLPV
LCQAHGLTPEQVVAIASNGGGKQALETVQALLPVLCQAHGLTPEQVVAIASNGGGKQALETVQALLP
VLCQAHGLTPEQVVAIASNIGGKQALETVQRLLPVLCQAHGLTPEQVVAIASNNGGKQALETVQALL
PVLCQAHGLTPEQVVAIASHDGGKQALETVQRLLPVLCQAHGLTPEQVVAIASNIGGKQALETVQAL
LPVLCQAHGLTPEQVVAIASNNGGKQALETVQALLPVLCQAHGLTPEQVVAIASNNGGKQALETVQA
LLPVLCQAHGLTPEQVVAIASNIGGKQALETVQRLLPVLCQAHGLTPQQVVAIASNIGGKQALETVQR
LLPVLCQAHGLTPQQVVAIASNGGGRPALESIVAQLSRPDPALAALTNDHLVALACLGGRPALDAVK
KGLG
ND5.2-DdCBE Right mitoTALE repeat (Note: Terminal NG RVD
recognizes a mismatched T instead of a G in the reference genome)
(SEQ ID NO: 161)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPQQVVAIASNNGGKQALETVQRLLPVLCQAHGLTPEQVVAIASHDGGKQALETVQRLLPV
LCQAHGLTPQQVVAIASNGGGKQALETVQRLLPVLCQAHGLTPEQVVAIASNIGGKQALETVQALLP
VLCQAHGLTPQQVVAIASNNGGKQALETVQRLLPVLCQAHGLTPQQVVAIASNNGGKQALETVQRL
LPVLCQAHGLTPEQVVAIASHDGGKQALETVQRLLPVLCQAHGLTPQQVVAIASNGGGKQALETVQR
LLPVLCQAHGLTPQQVVAIASNNGGKQALETVQRLLPVLCQAHGLTPEQVVAIASHDGGKQALETVQ
RLLPVLCQAHGLTPEQVVAIASHDGGKQALETVQRLLPVLCQAHGLTPEQVVAIASNIGGKQALETV
QALLPVLCQAHGLTPEQVVAIASNIGGKQALETVQALLPVLCQAHGLTPQQVVAIASNGGGKQALET
VQRLLPVLCQAHGLTPQQVVAIASNNGGKQALETVQRLLPVLCQAHGLTPQQVVAIASNGGGRPALE
SIVAQLSRPDPALAALTNDHLVALACLGGRPALDAVKKGLG
ND5.2-DdCBE Left mitoTALE repeat
(SEQ ID NO: 173)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASHDGGKQALETVQALLPVLCQAHGLTPQQVVAIASNIGGKQALETVQRLLPVL
CQAHGLTPQQVVAIASNIGGKQALETVQRLLPVLCQAHGLTPEQVVAIASNIGGKQALETVQALLPVL
CQAHGLTPEQVVAIASNIGGKQALETVQALLPVLCQAHGLTPEQVVAIASHDGGKQALETVQALLPV
LCQAHGLTPEQVVAIASHDGGKQALETVQRLLPVLCQAHGLTPEQVVAIASNIGGKQALETVQALLP
VLCQAHGLTPEQVVAIASNGGGKQALETVQRLLPVLCQAHGLTPEQVVAIASNIGGKQALETVQALL
PVLCQAHGLTPEQVVAIASHDGGKQALETVQALLPVLCQAHGLTPEQVVAIASHDGGKQALETVQAL
LPVLCQAHGLTPEQVVAIASNGGGKQALETVQRLLPVLCQAHGLTPQQVVAIASHDGGKQALETVQR
LLPVLCQAHGLTPQQVVAIASNGGGRPALESIVAQLSRPDPALAALTNDHLVALACLGGRPALDAVK
KGLG
ND5.3-DdCBE Right mitoTALE repeat
(SEQ ID NO: 165)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPQQVVAIASNIGGKQALETVQRLLPVLCQAHGLTPEQVVAIASNIGGKQALETVQRLLPVL
CQAHGLTPQQVVAIASNNGGKQALETVQRLLPVLCQAHGLTPEQVVAIASNIGGKQALETVQALLPV
LCQAHGLTPQQVVAIASNGGGKQALETVQRLLPVLCQAHGLTPQQVVAIASNGGGKQALETVQRLLP
VLCQAHGLTPEQVVAIASNIGGKQALETVQRLLPVLCQAHGLTPQQVVAIASNNGGKQALETVQRLL
PVLCQAHGLTPQQVVAIASNGGGKQALETVQRLLPVLCQAHGLTPEQVVAIASNIGGKQALETVQRL
LPVLCQAHGLTPEQVVAIASNGGGKQALETVQRLLPVLCQAHGLTPQQVVAIASNNGGKQALETVQR
LLPVLCQAHGLTPQQVVAIASNNGGKQALETVQRLLPVLCQAHGLTPQQVVAIASNGGGRPALESIV
AQLSRPDPALAALTNDHLVALACLGGRPALDAVKKGLG
ND5.3-DdCBE Left mitoTALE repeat
(SEQ ID NO: 167)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASHDGGKQALETVQALLPVLCQAHGLTPQQVVAIASNIGGKQALETVQRLLPVL
CQAHGLTPQQVVAIASNNGGKQALETVQRLLPVLCQAHGLTPEQVVAIASNGGGKQALETVQALLPV
LCQAHGLTPEQVVAIASNGGGKQALETVQALLPVLCQAHGLTPEQVVAIASHDGGKQALETVQALLP
VLCQAHGLTPEQVVAIASNGGGKQALETVQRLLPVLCQAHGLTPEQVVAIASNGGGKQALETVQALL
PVLCQAHGLTPEQVVAIASHDGGKQALETVQRLLPVLCQAHGLTPEQVVAIASNIGGKQALETVQAL
LPVLCQAHGLTPEQVVAIASNIGGKQALETVQALLPVLCQAHGLTPEQVVAIASNIGGKQALETVQAL
LPVLCQAHGLTPEQVVAIASNGGGKQALETVQRLLPVLCQAHGLTPQQVVAIASNIGGKQALETVQR
LLPVLCQAHGLTPQQVVAIASNGGGRPALESIVAQLSRPDPALAALTNDHLVALACLGGRPALDAVK
KGLG
ATP8-DdCBE Right mitoTALE repeat (Note: Terminal NG RVD recognizes
a mismatched T instead of a C in the reference genome)
(SEQ ID NO: 12)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPQQVVAIASNIGGKQALETVQRLLPVLCQAHGLTPEQVVAIASNGGGKQALETVQRLLPVL
CQAHGLTPQQVVAIASNNGGKQALETVQRLLPVLCQAHGLTPEQVVAIASNNGGKQALETVQALLPV
LCQAHGLTPQQVVAIASNNGGKQALETVQRLLPVLCQAHGLTPQQVVAIASHDGGKQALETVQRLLP
VLCQAHGLTPEQVVAIASNGGGKQALETVQRLLPVLCQAHGLTPQQVVAIASNGGGKQALETVQRLL
PVLCQAHGLTPQQVVAIASNGGGKQALETVQRLLPVLCQAHGLTPEQVVAIASNNGGKQALETVQRL
LPVLCQAHGLTPEQVVAIASNNGGKQALETVQRLLPVLCQAHGLTPQQVVAIASNGGGRPALESIVA
QLSRPDPALAALTNDHLVALACLGGRPALDAVKKGLG
ATP8-DdCBE Left mitoTALE repeat
(SEQ ID NO: 170)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASNIGGKQALETVQALLPVLCQAHGLTPQQVVAIASNGGGKQALETVQRLLPVL
CQAHGLTPQQVVAIASNGGGKQALETVQRLLPVLCQAHGLTPEQVVAIASNIGGKQALETVQALLPV
LCQAHGLTPEQVVAIASNIGGKQALETVQALLPVLCQAHGLTPEQVVAIASNIGGKQALETVQALLPV
LCQAHGLTPEQVVAIASHDGGKQALETVQRLLPVLCQAHGLTPEQVVAIASNIGGKQALETVQALLP
VLCQAHGLTPEQVVAIASHDGGKQALETVQRLLPVLCQAHGLTPEQVVAIASNIGGKQALETVQALL
PVLCQAHGLTPEQVVAIASNIGGKQALETVQALLPVLCQAHGLTPEQVVAIASNIGGKQALETVQALL
PVLCQAHGLTPEQVVAIASHDGGKQALETVQRLLPVLCQAHGLTPQQVVAIASNGGGKQALETVQRL
LPVLCQAHGLTPEQVVAIASNIGGKQALETVQALLPVLCQAHGLTPQQVVAIASNGGGRPALESIVAQ
LSRPDPALAALTNDHLVALACLGGRPALDAVKKGLG

### Base Editors Comprising MitoZFs

The Evolved DddA-containing base editors described herein contemplate fusion proteins comprising a mitoZF and an Evolved DddA domain or fragment or portion thereof (e.g., an N-terminal or C-terminal fragment or portion of a DddA), and optionally the joining of the two by a linker. The application contemplates any suitable mitoZF and an Evolved DddA domain to be combined in a single fusion protein. Examples of mitoZFs and DddA domains are each defined herein.

In some embodiments, the Evolved DddA-containing base editors comprise DddA domains which are DdCBE, i.e., DddA which deaminates a C. Examples of general architecture of Evolved DddA-containing base editors comprising DdCBEs and mitoZFs and their amino acid and nucleotide sequences are as follows:

| | | |
|---|---|---|
| R8 v6 | MLGFVGRVAAAPASGALRRLTPSASLPP | MTS |
| | AQLLLRAAPTAVHPVRDYAAQDYKDD | MLGFVGRVAAAPASGALRRLTPSASLPPAQLLL |
| | DDKVDEMTKKFGTLTIHDTEKGSLQKK | RAAPTAVHPVRDYAAQ (SEQ ID NO: 230) |
| | LEELELDAAMAERPFQCDICMRNFSTSG | FLAG tag |
| | SLSRHIRTHTGEKPFQCDICMRNFSQSGS | DYKDDDDK (SEQ ID NO: 347) |
| | LTRHIRTHTGSEKPFQCDICMRNFSRSDA | NES |
| | LSQHIRTHTGEKPFQCDICMRNFSRNDN | VDEMTKKFGTLTIHDTEK (SEQ ID NO: 232) |
| | RITHIRTHTGEKPFQCDICMRNFSRSDHL | Linker |
| | TQHTKIHLRGSGGGGSGGSGGSGSYAL | GS |
| | GPYQISAPQLPAYNGQTVGTFYYVNDA | NES2 |
| | GGLESKVFSSGGPTPYPNYANAGHVEG | LQKKLEELELD (SEQ ID NO: 233) |
| | QSALFMRDNGISEGLVFHNNPEGTCGFC | Linker |
| | VNMTETLLPENAKMTVVPPEGSGGSTN | AA |
| | LSDIIEKETGKQLVIQESILMLPEEVEEVI | ZF (R8) |
| | GNKPESDILVHTAYDESTDENVMLLTSD | MAERPFQCDICMRNFSTSGSLSRHIRTHTGEKPF |
| | APEYKPWALVIQDSNGENKIKMLGSGA | QCDICMRNFSQSGSLTRHIRTHTGSEKPFQCDIC |
| | TNFSLLKQAGDVEENPGPMASVLTPLLL | MRNFSRSDALSQHIRTHTGEKPFQCDICMRNFS |
| | RGLTGSARRLPVPRAKIHSLGSTNLSDII | RNDNRITHIRTHTGEKPFQCDICMRNFSRSDHL |
| | EKETGKQLVIQESILMLPEEVEEVIGNKP | TQHTKIHLR (SEQ ID NO: 224) |
| | ESDILVHTAYDESTDENVMLLTSDAPEY | Linker |
| | KPWALVIQDSNGENKIKML | GSGGGGSGGSGGS (SEQ ID NO: 222) |
| | (SEQ ID NO: 174) | Split DddA (DddA-G1397N) |
| | | GSYALGPYQISAPQLPAYNGQTVGTFYYVNDA |
| | | GGLESKVFSSGGPTPYPNYANAGHVEGQSALF |
| | | MRDNGISEGLVFHNNPEGTCGFCVNMTETLLP |
| | | ENAKMTVVPPEG (SEQ ID NO: 340) |
| | | Linker |
| | | SGGS (SEQ ID NO: 221) |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 378) |
| | | Linker |
| | | GSG |
| | | P2A Peptide |
| | | ATNFSLLKQAGDVEENPGP |
| | | (SEQ ID NO: 223) |
| | | MTS |
| | | MASVLTPLLLRGLTGSARRLPVPRAKIHSL |
| | | (SEQ ID NO: 231) |
| | | Linker |
| | | GS |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 378) |
| 5xZnF-4- | MLGFVGRVAAAPASGALRRLTPSASLPP | MTS |
| R8 v6 | AQLLLRAAPTAVHPVRDYAAQDYKDD | MLGFVGRVAAAPASGALRRLTPSASLPPAQLLL |
| | DDKVDEMTKKFGTLTIHDTEKGSLQKK | RAAPTA VHPVRDYAAQ (SEQ ID NO: 230) |
| | LEELELDAAMAERPFQCDICMRNFSQAS | FLAG tag |
| | NLISHIRTHTGEKPFQCDICMRNFSTSHS | DYKDDDDK (SEQ ID NO: 347) |
| | LTEHIRTHTGSEKPFQCDICMRNFSERSH | NES |
| | LREHIRTHTGEKPFQCDICMRNFSQSGN | VDEMTKKFGTLTIHDTEK (SEQ ID NO: 232) |
| | LTEHIRTHTGEKPFQCDICMRNFSSKKA | Linker |
| | LTEHTKIHLRGSGGGGSGGSGGSAIPVK | GS |
| | RGATGETKVFTGNSNSPKSPTKGGCSGG | NES2 |
| | STNLSDIIEKETGKQLVIQESILMLPEEVE | LQKKLEELELD (SEQ ID NO: 233) |
| | EVIGNKPESDILVHTAYDESTDENVMLL | Linker |
| | TSDAPEYKPWALVIQDSNGENKIKMLGS | AA |
| | GATNFSLLKQAGDVEENPGPMASVLTP | ZF (5xZnF-4-R8) |
| | LLLRGLTGSARRLPVPRAKIHSLGSTNLS | MAERPFQCDICMRNFSQASNLISHIRTHTGEKPF |
| | DIIEKETGKQLVIQESILMLPEEVEEVIGN | QCDICMRNFSTSHSLTEHIRTHTGSEKPFQCDIC |
| | KPESDILVHTAYDESTDENVMLLTSDAP | MRNFSERSHLREHIRTHTGEKPFQCDICMRNFS |
| | EYKPWALVIQDSNGENKIKML | QSGNLTEHIRTHTGEKPFQCDICMRNFSSKKAL |
| | (SEQ ID NO: 175) | TEHTKIHLR (SEQ ID NO: 225) |
| | | Linker |
| | | GSGGGGSGGSGGS (SEQ ID NO: 222) |
| | | Split DddA (DddA-G1397C) |
| | | AIPVKRGATGETKVFTGNSNSPKSPTKGGC |
| | | (SEQ ID NO: 341) |
| | | Linker |
| | | SGGS (SEQ ID NO: 221) |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 378) |
| | | Linker |
| | | GSG |
| | | P2A Peptide |
| | | ATNFSLLKQAGDVEENPGP (SEQ ID NO: 223) |
| | | MTS |
| | | MASVLTPLLLRGLTGSARRLPVPRAKIHSL |
| | | (SEQ ID NO: 231) |
| | | Linker |
| | | GS |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 378) |
| 5xZnF-10- | MLGFVGRVAAAPASGALRRLTPSASLPP | MTS |
| R8 v6 | AQLLLRAAPTAVHPVRDYAAQDYKDD | MLGFVGRVAAAPASGALRRLTPSASLPPAQLLL |
| | DDKVDEMTKKFGTLTIHDTEKGSLQKK | RAAPTAVHPVRDYAAQ (SEQ ID NO: 230) |
| | LEELELDAAMAERPFQCDICMRNFSQAS | FLAG tag |
| | NLISHIRTHTGEKPFQCDICMRNFSQRAN | DYKDDDDK (SEQ ID NO: 347) |
| | LRAHIRTHTGSEKPFQCDICMRNFSQAS | NES |
| | NLISHIRTHTGEKPFQCDICMRNFSTSHS | VDEMTKKFGTLTIHDTEK (SEQ ID NO: 232) |
| | LTEHIRTHTGEKPFQCDICMRNFSERSHL | Linker |
| | REHTKIHLRGSGGGGSGGSGGSAIPVKR | GS |
| | GATGETKVFTGNSNSPKSPTKGGCSGGS | NES2 |
| | TNLSDIIEKETGKQLVIQESILMLPEEVEE | LQKKLEELELD (SEQ ID NO: 233) |
| | VIGNKPESDILVHTAYDESTDENVMLLT | Linker |
| | SDAPEYKPWALVIQDSNGENKIKMLGS | AA |
| | GATNFSLLKQAGDVEENPGPMASVLTP | ZF (5xZnF-10-R8) |
| | LLLRGLTGSARRLPVPRAKIHSLGSTNLS | MAERPFQCDICMRNFSQASNLISHIRTHTGEKPF |
| | DIIEKETGKQLVIQESILMLPEEVEEVIGN | QCDICMRNFSQRANLRAHIRTHTGSEKPFQCDI |
| | KPESDILVHTAYDESTDENVMLLTSDAP | CMRNFSQASNLISHIRTHTGEKPFQCDICMRNFS |
| | EYKPWALVIQDSNGENKIKML | TSHSLTEHIRTHTGEKPFQCDICMRNFSERSHLR |
| | (SEQ ID NO: 176) | EHTKIHLR (SEQ ID NO: 226) |
| | | Linker |
| | | GSGGGGSGGSGGS (SEQ ID NO: 222) |
| | | Split DddA (DddA-G1397C) |
| | | AIPVKRGATGETKVFTGNSNSPKSPTKGGC |
| | | (SEQ ID NO: 341) |
| | | Linker |
| | | SGGS (SEQ ID NO: 221) |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 378) |
| | | Linker |
| | | GSG |
| | | P2A Peptide |
| | | ATNFSLLKQAGDVEENPGP (SEQ ID NO: 223) |
| | | MTS |
| | | MASVLTPLLLRGLTGSARRLPVPRAKIHSL |
| | | (SEQ ID NO: 231) |
| | | Linker |
| | | GS |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 378) |
| R13-1 v6 | MLGFVGRVAAAPASGALRRLTPSASLPP | MTS |
| | AQLLLRAAPTAVHPVRDYAAQDYKDD | MLGFVGRVAAAPASGALRRLTPSASLPPAQLLL |
| | DDKVDEMTKKFGTLTIHDTEKGSLQKK | RAAPTAVHPVRDYAAQ (SEQ ID NO: 230) |
| | LEELELDAAMAERPFQCDICMRNFSRSD | FLAG tag |
| | NLSTHIRTHTGEKPFQCDICMRNFSDRS | DYKDDDDK (SEQ ID NO: 347) |
| | DLSRHIRTHTGEKPFQCDICMRNFSQSG | NES |
| | DLTRHIRTHTGSEKPFQCDICMRNFSRSD | VDEMTKKFGTLTIHDTEK (SEQ ID NO: 232) |
| | SLSAHIRTHTGEKPFQCDICMRNFSQKA | Linker |
| | TRITHTKIHLRGSGGGGSGGSGGSGSYA | GS |
| | LGPYQISAPQLPAYNGQTVGTFYYVND | NES2 |
| | AGGLESKVFSSGGPTPYPNYANAGHVE | LQKKLEELELD (SEQ ID NO: 233) |
| | GQSALFMRDNGISEGLVFHNNPEGTCGF | Linker |
| | CVNMTETLLPENAKMTVVPPEGSGGST | AA |
| | NLSDIIEKETGKQLVIQESILMLPEEVEEV | ZF (R13-1) |
| | IGNKPESDILVHTAYDESTDENVMLLTS | MAERPFQCDICMRNFSRSDNLSTHIRTHTGEKP |
| | DAPEYKPWALVIQDSNGENKIKMLGSG | FQCDICMRNFSDRSDLSRHIRTHTGEKPFQCDIC |
| | ATNFSLLKQAGDVEENPGPMASVLTPLL | MRNFSQSGDLTRHIRTHTGSEKPFQCDICMRNF |
| | LRGLTGSARRLPVPRAKIHSLGSTNLSDI | SRSDSLSAHIRTHTGEKPFQCDICMRNFSQKAT |
| | IEKETGKQLVIQESILMLPEEVEEVIGNK | RITHTKIHLR (SEQ ID NO: 227) |
| | PESDILVHTAYDESTDENVMLLTSDAPE | Linker |
| | YKPWALVIQDSNGENKIKML | GSGGGGSGGSGGS (SEQ ID NO: 222) |
| | (SEQ ID NO: 177) | Split DddA (DddA-G1397N) |
| | | GSYALGPYQISAPQLPAYNGQTVGTFYYVNDA |
| | | GGLESKVFSSGGPTPYPNYANAGHVEGQSALF |
| | | MRDNGISEGLVFHNNPEGTCGFCVNMTETLLP |
| | | ENAKMTVVPPEG (SEQ ID NO: 340) |
| | | Linker |
| | | SGGS (SEQ ID NO: 221) |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 378) |
| | | Linker |
| | | GSG |
| | | P2A Peptide |
| | | ATNFSLLKQAGDVEENPGP (SEQ ID NO: 223) |
| | | MTS |
| | | MASVLTPLLLRGLTGSARRLPVPRAKIHSL |
| | | (SEQ ID NO: 231) |
| | | Linker |
| | | GS |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 378) |
| 5xZnF-9- | MLGFVGRVAAAPASGALRRLTPSASLPP | MTS |
| R13 v6 | AQLLLRAAPTAVHPVRDYAAQDYKDD | MLGFVGRVAAAPASGALRRLTPSASLPPAQLLL |
| | DDKVDEMTKKFGTLTIHDTEKGSLQKK | RAAPTAVHPVRDYAAQ (SEQ ID NO: 230) |
| | LEELELDAAMAERPFQCDICMRNFSQSS | FLAG tag |
| | SLVRHIRTHTGEKPFQCDICMRNFSRSD | DYKDDDDK (SEQ ID NO: 347) |
| | NLVRHIRTHTGSEKPFQCDICMRNFSQA | NES |
| | GHLASHIRTHTGEKPFQCDICMRNFSRK | LQKKLEELELD (SEQ ID NO: 233) |
| | DNLKNHIRTHTGEKPFQCDICMRNFSRK | VDEMTKKFGTLTIHDTEK |
| | DALRGHTKIHLRGSGGGGSGGSGGSAIP | (SEQ ID NO: 232) |
| | VKRGATGETKVFTGNSNSPKSPTKGGCS | Linker |
| | GGSTNLSDIIEKETGKQLVIQESILMLPEE | GS |
| | VEEVIGNKPESDILVHTAYDESTDENVM | NES2 |
| | LLTSDAPEYKPWALVIQDSNGENKIKML | Linker |
| | GSGATNFSLLKQAGDVEENPGPMASVL | AA |
| | TPLLLRGLTGSARRLPVPRAKIHSLGSTN | ZF (5xZnF-9-R13) |
| | LSDIIEKETGKQLVIQESILMLPEEVEEVI | MAERPFQCDICMRNFSQSSSLVRHIRTHTGEKP |
| | GNKPESDILVHTAYDESTDENVMLLTSD | FQCDICMRNFSRSDNLVRHIRTHTGSEKPFQCDI |
| | APEYKPWALVIQDSNGENKIKML | CMRNFSQAGHLASHIRTHTGEKPFQCDICMRNF |
| | (SEQ ID NO: 178) | SRKDNLKNHIRTHTGEKPFQCDICMRNFSRKDA |
| | | LRGHTKIHLR (SEQ ID NO: 228) |
| | | Linker |
| | | GSGGGGSGGSGGS (SEQ ID NO: 222) |
| | | Split DddA (DddA-G1397C) |
| | | AIPVKRGATGETKVFTGNSNSPKSPTKGGC |
| | | (SEQ ID NO: 341) |
| | | Linker |
| | | SGGS (SEQ ID NO: 221) |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 378) |
| | | Linker |
| | | GSG |
| | | P2A Peptide |
| | | ATNFSLLKQAGDVEENPGP (SEQ ID NO: 223) |
| | | MTS |
| | | MASVLTPLLLRGLTGSARRLPVPRAKIHSL |
| | | (SEQ ID NO: 231) |
| | | Linker |
| | | GS |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 378) |
| 5xZnF-12- | MLGFVGRVAAAPASGALRRLTPSASLPP | MTS |
| R13 v6 | AQLLLRAAPTAVHPVRDYAAQDYKDD | MLGFVGRVAAAPASGALRRLTPSASLPPAQLLL |
| | DDKVDEMTKKFGTLTIHDTEKGSLQKK | RAAPTAVHPVRDYAAQ (SEQ ID NO: 230) |
| | LEELELDAAMAERPFQCDICMRNFSRSD | FLAG tag |
| | HLTTHIRTHTGEKPFQCDICMRNFSQSSS | DYKDDDDK (SEQ ID NO: 347) |
| | LVRHIRTHTGSEKPFQCDICMRNFSRSD | NES |
| | NLVRHIRTHTGEKPFQCDICMRNFSQAG | VDEMTKKFGTLTIHDTEK (SEQ ID NO: 232) |
| | HLASHIRTHTGEKPFQCDICMRNFSRKD | Linker |
| | NLKNHTKIHLRGSGGGGSGGSGGSAIPV | GS |
| | KRGATGETKVFTGNSNSPKSPTKGGCSG | NES2 |
| | GSTNLSDIIEKETGKQLVIQESILMLPEEV | LQKKLEELELD (SEQ ID NO: 233) |
| | EEVIGNKPESDILVHTAYDESTDENVML | Linker |
| | LTSDAPEYKPWALVIQDSNGENKIKML | AA |
| | GSGATNFSLLKQAGDVEENPGPMASVL | ZF (5xZnF-12-R13) |
| | TPLLLRGLTGSARRLPVPRAKIHSLGSTN | MAERPFQCDICMRNFSRSDHLTTHIRTHTGEKP |
| | LSDIIEKETGKQLVIQESILMLPEEVEEVI | FQCDICMRNFSQSSSLVRHIRTHTGSEKPFQCDI |
| | GNKPESDILVHTAYDESTDENVMLLTSD | CMRNFSRSDNLVRHIRTHTGEKPFQCDICMRNF |
| | APEYKPWALVIQDSNGENKIKML | SQAGHLASHIRTHTGEKPFQCDICMRNFSRKDN |
| | (SEQ ID NO: 179) | LKNHTKIHLR (SEQ ID NO: 229) |
| | | Linker |
| | | GSGGGGSGGSGGS (SEQ ID NO: 222) |
| | | Split DddA (DddA-G1397C) |
| | | AIPVKRGATGETKVFTGNSNSPKSPTKGGC |
| | | (SEQ ID NO: 341) |
| | | Linker |
| | | SGGS (SEQ ID NO: 221) |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 378) |
| | | Linker |
| | | GSG |
| | | P2A Peptide |
| | | ATNFSLLKQAGDVEENPGP (SEQ ID NO: 223) |
| | | MTS |
| | | MASVLTPLLLRGLTGSARRLPVPRAKIHSL |
| | | (SEQ ID NO: 231) |
| | | Linker |
| | | GS |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 378) |
| R8 v3 | MLGFVGRVAAAPASGALRRLTPSASLPP | MTS |
| | AQLLLRAAPTAVHPVRDYAAQDYKDD | MLGFVGRVAAAPASGALRRLTPSASLPPAQLLL |
| | DDKVDEMTKKFGTLTIHDTEKAAMAER | RAAPTAVHPVRDYAAQ (SEQ ID NO: 230) |
| | PFQCRICMRNFSTSGSLSRHIRTHTGEKP | FLAG tag |
| | FACDICGRKFAQSGSLTRHTKIHTGGQR | DYKDDDDK (SEQ ID NO: 347) |
| | PFQCRICMRNFSRSDALSQHIRTHTGEKP | NES |
| | FACDICGRKFARNDNRITHTKIHTGEKPF | VDEMTKKFGTLTIHDTEK (SEQ ID NO: 232) |
| | QCRICMRKFARSDHLTQHTKIHLRGSGG | Linker |
| | GGSGGSGGSGSYALGPYQISAPQLPAYN | AA |
| | GQTVGTFYYVNDAGGLESKVFSSGGPT | ZF (R8) |
| | PYPNYANAGHVEGQSALFMRDNGISEG | MAERPFQCRICMRNFSTSGSLSRHIRTHTGEKPF |
| | LVFHNNPEGTCGFCVNMTETLLPENAK | ACDICGRKFAQSGSLTRHTKIHTGGQRPFQCRI |
| | MTVVPPEGSGGSTNLSDIIEKETGKQLVI | CMRNFSRSDALSQHIRTHTGEKPFACDICGRKF |
| | QESILMLPEEVEEVIGNKPESDILVHTAY | ARNDNRITHTKIHTGEKPFQCRICMRKFARSDH |
| | DESTDENVMLLTSDAPEYKPWALVIQD | LTQHTKIHLR (SEQ ID NO: 400) |
| | SNGENKIKML (SEQ ID NO: 180) | Linker |
| | | GSGGGGSGGSGGS (SEQ ID NO: 222) |
| | | Split DddA (DddA-G1397N) |
| | | GSYALGPYQISAPQLPAYNGQTVGTFYYVNDA |
| | | GGLESKVFSSGGPTPYPNYANAGHVEGQSALF |
| | | MRDNGISEGLVFHNNPEGTCGFCVNMTETLLP |
| | | ENAKMTVVPPEG (SEQ ID NO: 340) |
| | | Linker |
| | | SGGS (SEQ ID NO: 221) |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 378) |
| 5xZnF-4- | MLGFVGRVAAAPASGALRRLTPSASLPP | MTS |
| R8 v3 | AQLLLRAAPTAVHPVRDYAAQDYKDD | MLGFVGRVAAAPASGALRRLTPSASLPPAQLLL |
| | DDKVDEMTKKFGTLTIHDTEKAAMAER | RAAPTAVHPVRDYAAQ (SEQ ID NO: 230) |
| | PFQCRICMRNFSQASNLISHIRTHTGEKP | FLAG tag |
| | FACDICGRKFATSHSLTEHTKIHTGSQKP | DYKDDDDK (SEQ ID NO: 347) |
| | FQCRICMRNFSERSHLREHIRTHTGEKPF | NES |
| | ACDICGRKFAQSGNLTEHTKIHTGEKPF | VDEMTKKFGTLTIHDTEK (SEQ ID NO: 232) |
| | QCRICMRKFASKKALTEHTKIHLRGSGG | Linker |
| | GGSGGSGGSAIPVKRGATGETKVFTGNS | AA |
| | NSPKSPTKGGCSGGSTNLSDIIEKETGKQ | ZF (5xZnF-4-R8) |
| | LVIQESILMLPEEVEEVIGNKPESDILVHT | MAERPFQCRICMRNFSQASNLISHIRTHTGEKPF |
| | AYDESTDENVMLLTSDAPEYKPWALVI | ACDICGRKFATSHSLTEHTKIHTGSQKPFQCRIC |
| | QDSNGENKIKML (SEQ ID NO: 181) | MRNFSERSHLREHIRTHTGEKPFACDICGRKFA |
| | | QSGNLTEHTKIHTGEKPFQCRICMRKFASKKAL |
| | | TEHTKIHLR (SEQ ID NO: 401) |
| | | Linker |
| | | GSGGGGSGGSGGS (SEQ ID NO: 222) |
| | | Split DddA (DddA-G1397C) |
| | | AIPVKRGATGETKVFTGNSNSPKSPTKGGC |
| | | (SEQ ID NO: 341) |
| | | Linker |
| | | SGGS (SEQ ID NO: 221) |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 378) |
| 5xZnF-10- | MLGFVGRVAAAPASGALRRLTPSASLPP | MTS |
| R8 v3 | AQLLLRAAPTAVHPVRDYAAQDYKDD | MLGFVGRVAAAPASGALRRLTPSASLPPAQLLL |
| | DDKVDEMTKKFGTLTIHDTEKAAMAER | RAAPTAVHPVRDYAAQ (SEQ ID NO: 230) |
| | PFQCRICMRNFSQASNLISHIRTHTGEKP | FLAG tag |
| | FACDICGRKFAQRANLRAHTKIHTGSQK | DYKDDDDK (SEQ ID NO: 347) |
| | PFQCRICMRNFSQASNLISHIRTHTGEKP | NES |
| | FACDICGRKFATSHSLTEHTKIHTGEKPF | VDEMTKKFGTLTIHDTEK (SEQ ID NO: 232) |
| | QCRICMRKFAERSHLREHTKIHLRGSGG | Linker |
| | GGSGGSGGSAIPVKRGATGETKVFTGNS | AA |
| | NSPKSPTKGGCSGGSTNLSDIIEKETGKQ | ZF (5xZnF-10-R8) |
| | LVIQESILMLPEEVEEVIGNKPESDILVHT | MAERPFQCRICMRNFSQASNLISHIRTHTGEKPF |
| | AYDESTDENVMLLTSDAPEYKPWALVI | ACDICGRKFAQRANLRAHTKIHTGSQKPFQCRI |
| | QDSNGENKIKML (SEQ ID NO: 182) | CMRNFSQASNLISHIRTHTGEKPFACDICGRKFA |
| | | TSHSLTEHTKIHTGEKPFQCRICMRKFAERSHLR |
| | | EHTKIHLR (SEQ ID NO: 402) |
| | | Linker |
| | | GSGGGGSGGSGGS (SEQ ID NO: 222) |
| | | Split DddA (DddA-G1397C) |
| | | AIPVKRGATGETKVFTGNSNSPKSPTKGGC |
| | | (SEQ ID NO: 341) |
| | | Linker |
| | | SGGS (SEQ ID NO: 221) |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 378) |
| R13-1 v3 | MLGFVGRVAAAPASGALRRLTPSASLPP | MTS |
| | AQLLLRAAPTAVHPVRDYAAQDYKDD | MLGFVGRVAAAPASGALRRLTPSASLPPAQLLL |
| | DDKVDEMTKKFGTLTIHDTEKAAMAER | RAAPTAVHPVRDYAAQ (SEQ ID NO: 230) |
| | PFQCRICMRNFSRSDNLSTHIRTHTGEKP | FLAG tag |
| | FACDICGRKFADRSDLSRHTKIHTGEKPF | DYKDDDDK (SEQ ID NO: 347) |
| | QCRICMRKFAQSGDLTRHTKIHTGSQKP | NES |
| | FQCRICMRNFSRSDSLSAHIRTHTGEKPF | VDEMTKKFGTLTIHDTEK (SEQ ID NO: 232) |
| | ACDICGRKFAQKATRITHTKIHLRGSGG | Linker |
| | GGSGGSGGSGSYALGPYQISAPQLPAYN | AA |
| | GQTVGTFYYVNDAGGLESKVFSSGGPT | ZF (R13-1) |
| | PYPNYANAGHVEGQSALFMRDNGISEG | MAERPFQCRICMRNFSRSDNLSTHIRTHTGEKP |
| | LVFHNNPEGTCGFCVNMTETLLPENAK | FACDICGRKFADRSDLSRHTKIHTGEKPFQCRIC |
| | MTVVPPEGSGGSTNLSDIIEKETGKQLVI | MRKFAQSGDLTRHTKIHTGSQKPFQCRICMRNF |
| | QESILMLPEEVEEVIGNKPESDILVHTAY | SRSDSLSAHIRTHTGEKPFACDICGRKFAQKAT |
| | DESTDENVMLLTSDAPEYKPWALVIQD | RITHTKIHLR (SEQ ID NO: 403) |
| | SNGENKIKML (SEQ ID NO: 183) | Linker |
| | | GSGGGGSGGSGGS (SEQ ID NO: 222) |
| | | Split DddA (DddA-G1397N) |
| | | GSYALGPYQISAPQLPAYNGQTVGTFYYVNDA |
| | | GGLESKVFSSGGPTPYPNYANAGHVEGQSALF |
| | | MRDNGISEGLVFHNNPEGTCGFCVNMTETLLP |
| | | ENAKMTVVPPEG (SEQ ID NO: 340) |
| | | Linker |
| | | SGGS (SEQ ID NO: 221) |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 378) |
| 5xZnF-9- | MLGFVGRVAAAPASGALRRLTPSASLPP | MTS |
| R13 v3 | AQLLLRAAPTAVHPVRDYAAQDYKDD | MLGFVGRVAAAPASGALRRLTPSASLPPAQLLL |
| | DDKVDEMTKKFGTLTIHDTEKAAMAER | RAAPTAVHPVRDYAAQ (SEQ ID NO: 230) |
| | PFQCRICMRNFSQSSSLVRHIRTHTGEKP | FLAG tag |
| | FACDICGRKFARSDNLVRHTKIHTGSQK | DYKDDDDK (SEQ ID NO: 347) |
| | PFQCRICMRNFSQAGHLASHIRTHTGEK | NES |
| | PFACDICGRKFARKDNLKNHTKIHTGEK | VDEMTKKFGTLTIHDTEK (SEQ ID NO: 232) |
| | PFQCRICMRKFARKDALRGHTKIHLRGS | Linker |
| | GGGGSGGSGGSAIPVKRGATGETKVFT | AA |
| | GNSNSPKSPTKGGCSGGSTNLSDIIEKET | ZF (5xZnF-9-R13) |
| | GKQLVIQESILMLPEEVEEVIGNKPESDI | MAERPFQCRICMRNFSQSSSLVRHIRTHTGEKP |
| | LVHTAYDESTDENVMLLTSDAPEYKPW | FACDICGRKFARSDNLVRHTKIHTGSQKPFQCR |
| | ALVIQDSNGENKIKML | ICMRNFSQAGHLASHIRTHTGEKPFACDICGRK |
| | (SEQ ID NO: 184) | FARKDNLKNHTKIHTGEKPFQCRICMRKFARK |
| | | DALRGHTKIHLR (SEQ ID NO: 404) |
| | | Linker |
| | | GSGGGGSGGSGGS (SEQ ID NO: 222) |
| | | Split DddA (DddA-G1397C) |
| | | AIPVKRGATGETKVFTGNSNSPKSPTKGGC |
| | | (SEQ ID NO: 341) |
| | | Linker |
| | | SGGS (SEQ ID NO: 221) |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 378) |
| 5xZnF-12- | MLGFVGRVAAAPASGALRRLTPSASLPP | MTS |
| R13 v3 | AQLLLRAAPTAVHPVRDYAAQDYKDD | MLGFVGRVAAAPASGALRRLTPSASLPPAQLLL |
| | DDKVDEMTKKFGTLTIHDTEKAAMAER | RAAPTAVHPVRDYAAQ (SEQ ID NO: 230) |
| | PFQCRICMRNFSRSDHLTTHIRTHTGEKP | FLAG tag |
| | FACDICGRKFAQSSSLVRHTKIHTGSQKP | DYKDDDDK (SEQ ID NO: 347) |
| | FQCRICMRNFSRSDNLVRHIRTHTGEKP | NES |
| | FACDICGRKFAQAGHLASHTKIHTGEKP | VDEMTKKFGTLTIHDTEK (SEQ ID NO: 232) |
| | FQCRICMRKFARKDNLKNHTKIHLRGSG | Linker |
| | GGGSGGSGGSAIPVKRGATGETKVFTG | AA |
| | NSNSPKSPTKGGCSGGSTNLSDIIEKETG | ZF (5xZnF-12-R13) |
| | KQLVIQESILMLPEEVEEVIGNKPESDIL | MAERPFQCRICMRNFSRSDHLTTHIRTHTGEKP |
| | VHTAYDESTDENVMLLTSDAPEYKPWA | FACDICGRKFAQSSSLVRHTKIHTGSQKPFQCRI |
| | LVIQDSNGENKIKML | CMRNFSRSDNLVRHIRTHTGEKPFACDICGRKF |
| | (SEQ ID NO: 185) | AQAGHLASHTKIHTGEKPFQCRICMRKFARKD |
| | | NLKNHTKIHLR (SEQ ID NO: 405) |
| | | Linker |
| | | GSGGGGSGGSGGS (SEQ ID NO: 222) |
| | | Split DddA (DddA-G1397C) |
| | | AIPVKRGATGETKVFTGNSNSPKSPTKGGC |
| | | (SEQ ID NO: 341) |
| | | Linker |
| | | SGGS (SEQ ID NO: 221) |
| | | UGI |
| | | TNLSDIIEKETGKQLVIQESILMLPEEVEEVIGNK |
| | | PESDILVHTAYDESTDENVMLLTSDAPEYKPW |
| | | ALVIQDSNGENKIKML (SEQ ID NO: 378) |

### IV. Linkers

In certain embodiments, linkers may be used to link any of the peptides or peptide domains or moieties of the invention (e.g., a mitoTALE fused to an evolved DddA).

As defined above, the term “linker,” as used herein, refers to a chemical group or a molecule linking two molecules or moieties (e.g., a binding domain (e.g., mitoTALE) and a editing domain (e.g., DddA, or portion thereof)). In some embodiments, a linker joins a binding domain (e.g., mitoTALE) and a catalytic domain (e.g., DddA, or portion thereof). In some embodiments, a linker joins a mitoTALE and DddA. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 1-100 amino acids in length, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer linkers are also contemplated.

The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may included functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some other embodiments, the linker comprises the amino acid sequence is greater than one amino acid residues in length. In some embodiments, the linker comprises less than six amino acid in length. In some embodiments, the linker is two amino acid residues in length. In some embodiments, the linker comprises the amino acid sequence of any one of SEQ ID NOs.: 202-221.

In certain embodiments, linkers may be used to link any of the protein or protein domains described herein (e.g., a deaminase domain and a Cas9 domain). The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some embodiments, the linker is an amino acid or a plurality of amino acids (e.g., a peptide or protein). In some embodiments, the linker is a bond e.g., a covalent bond), an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35-40, 40-45, 45-50, 50-60, 60-70, 70-80, 80-90, 90-100, 100-110, 110-120, 120-130, 130-140, 140-150, or 150-200 amino acids in length. Longer or shorter linkers are also contemplated. In some embodiments, a linker comprises the amino acid sequence SGSETPGTSESATPES (SEQ ID NO: 202), which may also be referred to as the XTEN linker. In some embodiments, the linker is 32 amino acids in length. In some embodiments, the linker comprises the amino acid sequence (SGGS)₂-SGSETPGTSESATPES-(SGGS)₂ (SEQ ID NO: 203), which may also be referred to as (SGGS)₂-XTEN-(SGGS)₂ (SEQ ID NO: 203). In some embodiments, the linker comprises the amino acid sequence, wherein n is 0, 1, 2, 3, 4, 5, 6, 7, 8, 9, or 10. In some embodiments, a linker comprises the amino acid sequence SGGS (SEQ ID NO: 204). In some embodiments, a linker comprises (SGGS)ₙ (SEQ ID NO: 204), (GGGS)ₙ (SEQ ID NO: 205), (GGGGS)ₙ (SEQ ID NO: 206), (G)ₙ (SEQ ID NO: 207), (EAAAK)ₙ (SEQ ID NO: 208), (SGGS)ₙ-SGSETPGTSESATPES-(SGGS)ₙ (SEQ ID NO: 209), (GGS)ₙ (SEQ ID NO: 210), SGSETPGTSESATPES (SEQ ID NO: 202), or (XP)ₙ (SEQ ID NO: 211) motif, or a combination of any of these, wherein n is independently an integer between 1 and 30, and wherein X is any amino acid. In some embodiments, n is 1, 2, 3, 4, 5, 6, 7, 8, 9, 10, 11, 12, 13, 14, or 15. In some embodiments, a linker comprises SGSETPGTSESATPES (SEQ ID NO: 202), and SGGS (SEQ ID NO: 204). In some embodiments, a linker comprises SGGSSGSETPGTSESATPESSGGS (SEQ ID NO: 212). In some embodiments, a linker comprises SGGSSGGSSGSETPGTSESATPESSGGSSGGS (SEQ ID NO: 203). In some embodiments, a linker comprises GGSGGSPGSPAGSPTSTEEGTSESATPESGPGTSTEPSEGSAPGSPAGSPTSTEEGTSTEPSEGS APGTSTEPSEGSAPGTSESATPESGPGSEPATSGGSGGS (SEQ ID NO: 213). In some embodiments, the linker is 24 amino acids in length. In some embodiments, the linker comprises the amino acid sequence SGGSSGGSSGSETPGTSESATPES (SEQ ID NO: 214). In some embodiments, the linker is 40 amino acids in length. In some embodiments, the linker comprises the amino acid sequence SGGSSGGSSGSETPGTSESATPESSGGSSGGSSGGSSGGS (SEQ ID NO: 215). In some embodiments, the linker is 64 amino acids in length. In some embodiments, the linker comprises the amino acid sequence SGGSSGGSSGSETPGTSESATPESSGGSSGGSSGGSSGGSSGSETPGTSESATPESSGGSSGGS (SEQ ID NO: 216). In some embodiments, the linker is 92 amino acids in length. In some embodiments, the linker comprises the amino acid sequence PGSPAGSPTSTEEGTSESATPESGPGTSTEPSEGSAPGSPAGSPTSTEEGTSTEPSEGSAPGTST EPSEGSAPGTSESATPESGPGSEPATS (SEQ ID NO: 217). It should be appreciated that any of the linkers provided herein may be used to link a first adenosine deaminase and a second adenosine deaminase; an adenosine deaminase (e.g., a first or a second adenosine deaminase) and a napDNAbp; a napDNAbp and an NLS; or an adenosine deaminase (e.g., a first or a second adenosine deaminase) and an NLS.

In some embodiments, any of the fusion proteins provided herein, comprise an adenosine or a cytidine deaminase and a napDNAbp that are fused to each other via a linker. In some embodiments, any of the fusion proteins provided herein, comprise a first adenosine deaminase and a second adenosine deaminase that are fused to each other via a linker. In some embodiments, any of the fusion proteins provided herein, comprise an NLS, which may be fused to an adenosine deaminase (e.g., a first and/or a second adenosine deaminase), a nucleic acid programmable DNA binding protein (napDNAbp). Various linker lengths and flexibilities between an adenosine deaminase (e.g., an engineered ecTadA) and a napDNAbp (e.g., a Cas9 domain), and/or between a first adenosine deaminase and a second adenosine deaminase can be employed (e.g., ranging from very flexible linkers of the form (GGGGS)ₙ (SEQ ID NO: 206) and (G)ₙ (SEQ ID NO: 207) to more rigid linkers of the form (EAAAK)ₙ (SEQ ID NO: 208), (SGGS)ₙ (SEQ ID NO: 204), SGSETPGTSESATPES (SEQ ID NO: 202) (see, e.g., Guilinger J P, Thompson D B, Liu D R. Fusion of catalytically inactive Cas9 to FokI nuclease improves the specificity of genome modification. Nat. Biotechnol. 2014; 32(6): 577-82; the entire contents are incorporated herein by reference) and (XP)ₙ (SEQ ID NO: 211)) in order to achieve the optimal length for deaminase activity for the specific application. In some embodiments, n is 1, 2, 3, 4, 5, 6, 7, 8, 9, 10, 11, 12, 13, 14, or 15. In some embodiments, the linker comprises a (GGS)ₙ (SEQ ID NO: 210) motif, wherein n is 1, 3, or 7. In some embodiments, the adenosine deaminase and the napDNAbp, and/or the first adenosine deaminase and the second adenosine deaminase of any of the fusion proteins provided herein are fused via a linker comprising the amino acid sequence SGSETPGTSESATPES (SEQ ID NO: 1202), SGGS (SEQ ID NO: 104), SGGSSGSETPGTSESATPESSGGS (SEQ ID NO: 212), SGGSSGGSSGSETPGTSESATPESSGGSSGGS (SEQ ID NO: 203), or GGSGGSPGSPAGSPTSTEEGTSESATPESGPGTSTEPSEGSAPGSPAGSPTSTEEGTSTEPSEGS APGTSTEPSEGSAPGTSESATPESGPGSEPATSGGSGGS (SEQ ID NO: 213). In some embodiments, the linker is 24 amino acids in length. In some embodiments, the linker comprises the amino acid sequence SGGSSGGSSGSETPGTSESATPES (SEQ ID NO: 214). In some embodiments, the linker is 32 amino acids in length. In some embodiments, the linker is 32 amino acids in length. In some embodiments, the linker comprises the amino acid sequence (SGGS)₂-SGSETPGTSESATPES-(SGGS)₂ (SEQ ID NO: 203), which may also be referred to as (SGGS)₂-XTEN-(SGGS)₂ (SEQ ID NO: 203). In some embodiments, the linker comprises the amino acid sequence, wherein n is 0, 1, 2, 3, 4, 5, 6, 7, 8, 9, or 10. In some embodiments, the linker is 40 amino acids in length. In some embodiments, the linker comprises the amino acid sequence SGGSSGGSSGSETPGTSESATPESSGGSSGGSSGGSSGGS (SEQ ID NO: 215). In some embodiments, the linker is 64 amino acids in length. In some embodiments, the linker comprises the amino acid sequence SGGSSGGSSGSETPGTSESATPESSGGSSGGSSGGSSGGSSGSETPGTSESATPESSGGSSGGS (SEQ ID NO: 216). In some embodiments, the linker is 92 amino acids in length. In some embodiments, the linker comprises the amino acid sequence PGSPAGSPTSTEEGTSESATPESGPGTSTEPSEGSAPGSPAGSPTSTEEGTSTEPSEGSAPGTST EPSEGSAPGTSESATPESGPGSEPATS (SEQ ID NO: 217).

### V. Other Fusion Protein Domains

In various embodiments, the Evolved DddA-containing base editors or the polypeptides that comprise the Evolved DddA-containing base editors (e.g., the pDNAbps and DddA) may include one or more other functional domains, including uracil glycosylase inhibitors (UGI), NLS domains, and mitochondrial localization domains.

### Uracil Glycosylase Inhibitor (UGI)

In some embodiments, the fusion proteins of the disclosure comprise one or more UGI domains. When the DddA enzyme is employed and deaminates the target nucleotide, it may trigger uracil repair activity in the cell, thereby causing excision of the deaminated nucleotide. This may cause degradation of the nucleic acid or otherwise inhibit the effect of the correction or nucleotide alteration induced by the fusion protein. To inhibit this activity, a UGI may be desired. In some embodiments, the first and/or second fusion protein comprises more than one UGI. In some embodiments, the first and/or second fusion protein comprises two UGIs. In some embodiments, the first and/or second fusion protein contains two UGIs. The UGI or multiple UGIs may be appended or attached to any portion of the fusion protein. In some embodiments, the UGI is attached to the first or second portion of a DddA in the first or second fusion protein. In some embodiments, a second UGI is attached to the first UGI which is attached to the first or second portion of a DddA in the first or second fusion protein.

In other embodiments, the base editors described herein may comprise one or more uracil glycosylase inhibitors. The term “uracil glycosylase inhibitor” or “UGI,” as used herein, refers to a protein that is capable of inhibiting a uracil-DNA glycosylase base-excision repair enzyme. In some embodiments, a UGI domain comprises a wild-type UGI or a UGI as set forth in SEQ ID NO: 377.

In some embodiments, the UGI proteins provided herein include fragments of UGI and proteins homologous to a UGI or a UGI fragment. For example, in some embodiments, a UGI domain comprises a fragment of the amino acid sequence set forth in SEQ ID NO: 377. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 377. In some embodiments, a UGI comprises an amino acid sequence homologous to the amino acid sequence set forth in SEQ ID NO: 377, or an amino acid sequence homologous to a fragment of the amino acid sequence set forth in SEQ ID NO: 377. In some embodiments, proteins comprising UGI or fragments of UGI or homologs of UGI or UGI fragments are referred to as “UGI variants.” A UGI variant shares homology to UGI, or a fragment thereof. For example a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 377. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 377. In some embodiments, the UGI comprises the following amino acid sequence:

Uracil-DNA glycosylase inhibitor
(>sp|P14739|UNGI_BPPB2)
(SEQ ID NO: 377)
MTNLSDIIEKETGKQLVIQESILMLPEEVEEVIGNKPESDILVHTAYDES
TDENVMLLTSDAPEYKPWALVIQDSNGENKIKML.

The base editors described herein may comprise more than one UGI domain, which may be separated by one or more linkers as described herein. It will also be understood that in the context of the herein disclosed base editors, the UGI domain may be linked to a deaminase domain.

In some embodiments, a UGI is absent from a base editor. In some embodiments, where a base editor comprises a ZFP or mitoZFP, UGIs are removed or are absent from the base editor. In some embodiments, the removal and/or absence of UGIs increases the activity of a DddA.

### NLS Domains

In various embodiments, the fusion proteins may comprise one or more nuclear localization sequences (NLS), which help promote translocation of a protein into the cell nucleus. Such sequences are well-known in the art and can include the following examples:

| | | SEQ |
|---|---|---|
| | | ID |
| Description | Sequence | NO: |
| NLS of SV40 | PKKKRKV | 412 |
| large T-Ag | | |
| NLS of | VSRKRPRP | 413 |
| polyoma | | |
| large T-Ag | | |
| NLS of c-MYC | PAAKRVKLD | 414 |
| NLS of TUS- | KLKIKRPVK | 415 |
| protein | | |
| NLS of | EGAPPAKRAR | 416 |
| Hepatitis | | |
| D virus | | |
| antigen | | |
| NLS of murine | PPQPKKKPLDGE | 417 |
| p53 | | |
| NLS | MKRTADGSEFESPKKKRKV | 418 |
| NLS of | AVKRPAATKKAGQAKKKKLD | 419 |
| nucleoplasmin | | |
| NLS of PE1 | SGGSKRTADGSEFEPKKKRKV | 420 |
| and PE2 | | |
| NLS of EGL-13 | MSRRRKANPTKLSENAKKLAKEVEN | 421 |
| NLS | MDSLLMNRRKFLYQFKNVRWAKGRRETYLC | 422 |

The NLS examples above are non-limiting. The PE fusion proteins may comprise any known NLS sequence, including any of those described in Cokol et al., “Finding nuclear localization signals,” EMBO Rep., 2000, 1(5): 411-415 and Freitas et al., “Mechanisms and Signals for the Nuclear Import of Proteins,” Current Genomics, 2009, 10(8): 550-7, each of which are incorporated herein by reference.

### Mitochondrial Targeting Sequence (MTS)

In various embodiments, the Evolved DddA-containing base editors or the polypeptides that comprise the Evolved DddA-containing base editors (e.g., the pDNAbps and DddA) may be engineered to include one or more mitochondrial targeting sequences (MTS) (or mitochondrial localization sequence (MLS)) which facilitate that translocation of a polypeptide into the mitochondria. MTS are known in the art and exemplary sequences are provided herein. In general MTSs are short peptide sequences (about 3-70 amino acids long) that direct a newly synthesized protein to the mitochondria within a cell. It is usually found at the N-terminus and consists of an alternating pattern of hydrophobic and positively charged amino acids to form what is called an amphipathic helix. Mitochondrial localization sequences can contain additional signals that subsequently target the protein to different regions of the mitochondria, such as the mitochondrial matrix. One exemplary mitochondrial localization sequence is the mitochondrial localization sequence derived from Cox8, a mitochondrial cytochrome c oxidase subunit VIII. In embodiments, a mitochondrial localization sequence derived from Cox8 includes the amino acid sequence: MSVLTPLLLRGLTGSARRLPVPRAKIHSL (SEQ ID NO: 14). In the embodiments, the mitochondrial localization sequence derived from Cox8 includes an amino acid sequence that is about 50%, 55%, 60%, 65%, 70%, 75%, 80%, 85%, 90% or 95% identity to SEQ ID NO: 14.

### VI. Delivery

In another aspect, the present disclosure provides for the delivery of fusion proteins in vitro and in vivo using split DddA protein formulations. The presently disclosed methods for delivering fusion proteins via various methods. For example, DddA proteins have exhibited toxic effects in vivo, and so require special solutions. One such solution is formulating the DddA, and fusion protein thereof, split into pairs that are packaged into two separate rAAV particles that, when co-delivered to a cell, reconstitute the functional DddA protein. Several other special considerations to account for the unique features of fusion protein are described, including the optimization of split sites. MitoTALE-DddA and/or mitoZF-DddA and/or Cas9-DddA fusion proteins, mRNA expressing the fusion proteins, or DNA can be packaged into lipid nanoparticles, rAAV, or lentivirus and injected, ingested, or inhaled to alter genomic DNA in vivo and ex vivo, including for the purposes of establishing animal models of human disease, testing therapeutic and scientific hypotheses in animal models of human disease, and treating disease in humans.

In another aspect, the present disclosure provides for the delivery of base editors in vitro and in vivo using various strategies, including on separate vectors using split inteins and as well as direct delivery strategies of the ribonucleoprotein complex (i.e., the base editor complexed to the gRNA and/or the second-site gRNA) using techniques such as electroporation, use of cationic lipid-mediated formulations, and induced endocytosis methods using receptor ligands fused to the ribonucleoprotein complexes. Any such methods are contemplated herein.

In some aspects, the invention provides methods comprising delivering one or more base editor-encoding polynucleotides, such as or one or more vectors as described herein encoding one or more components of the base editing system described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a base editor as described herein in combination with (and optionally complexed with) a guide sequence is delivered to a cell. Conventional viral and non-viral based gene transfer methods can be used to introduce nucleic acids in mammalian cells or target tissues. Such methods can be used to administer nucleic acids encoding components of a base editor to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bihm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a viruses can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

In various embodiments, the base editor constructs (including, the split-constructs) may be engineered for delivery in one or more rAAV vectors. An rAAV as related to any of the methods and compositions provided herein may be of any serotype including any derivative or pseudotype (e.g., 1, 2, 3, 4, 5, 6, 7, 8, 9, 10, 11, 12, 13, 2/1, 2/5, 2/8, 2/9, 3/1, 3/5, 3/8, or 3/9). An rAAV may comprise a genetic load (i.e., a recombinant nucleic acid vector that expresses a gene of interest, such as a whole or split base editor fusion protein that is carried by the rAAV into a cell) that is to be delivered to a cell. An rAAV may be chimeric.

As used herein, the serotype of an rAAV refers to the serotype of the capsid proteins of the recombinant virus. Non-limiting examples of derivatives and pseudotypes include rAAV2/1, rAAV2/5, rAAV2/8, rAAV2/9, AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y->F), AAV8 (Y733F), AAV2.15, AAV2.4, AAVM41, and AAVr3.45. A non-limiting example of derivatives and pseudotypes that have chimeric VP1 proteins is rAAV2/5-1VP1u, which has the genome of AAV2, capsid backbone of AAV5 and VP1u of AAV1. Other non-limiting example of derivatives and pseudotypes that have chimeric VP1 proteins are rAAV2/5-8VP1u, rAAV2/9-1VP1u, and rAAV2/9-8VP1u.

AAV derivatives/pseudotypes, and methods of producing such derivatives/pseudotypes are known in the art (see, e.g., Mol Ther. 2012 April; 20(4):699-708. doi: 10.1038/mt.2011.287. Epub 2012 Jan. 24. The AAV vector toolkit: poised at the clinical crossroads. Asokan A1, Schaffer D V , Samulski R J.). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Methods of making or packaging rAAV particles are known in the art and reagents are commercially available (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). For example, a plasmid comprising a gene of interest may be combined with one or more helper plasmids, e.g., that contain a rep gene (e.g., encoding Rep78, Rep68, Rep52 and Rep40) and a cap gene (encoding VP1, VP2, and VP3, including a modified VP2 region as described herein), and transfected into a recombinant cells such that the rAAV particle can be packaged and subsequently purified.

Recombinant AAV may comprise a nucleic acid vector, which may comprise at a minimum: (a) one or more heterologous nucleic acid regions comprising a sequence encoding a protein or polypeptide of interest or an RNA of interest (e.g., a siRNA or microRNA), and (b) one or more regions comprising inverted terminal repeat (ITR) sequences (e.g., wild-type ITR sequences or engineered ITR sequences) flanking the one or more nucleic acid regions (e.g., heterologous nucleic acid regions). Herein, heterologous nucleic acid regions comprising a sequence encoding a protein of interest or RNA of interest are referred to as genes of interest.

Any one of the rAAV particles provided herein may have capsid proteins that have amino acids of different serotypes outside of the VP1u region. In some embodiments, the serotype of the backbone of the VP1 protein is different from the serotype of the ITRs and/or the Rep gene. In some embodiments, the serotype of the backbone of the VP1 capsid protein of a particle is the same as the serotype of the ITRs. In some embodiments, the serotype of the backbone of the VP1 capsid protein of a particle is the same as the serotype of the Rep gene. In some embodiments, capsid proteins of rAAV particles comprise amino acid mutations that result in improved transduction efficiency.

In some embodiments, the nucleic acid vector comprises one or more regions comprising a sequence that facilitates expression of the nucleic acid (e.g., the heterologous nucleic acid), e.g., expression control sequences operatively linked to the nucleic acid. Numerous such sequences are known in the art. Non-limiting examples of expression control sequences include promoters, insulators, silencers, response elements, introns, enhancers, initiation sites, termination signals, and poly(A) tails. Any combination of such control sequences is contemplated herein (e.g., a promoter and an enhancer).

Final AAV constructs may incorporate a sequence encoding the gRNA. In other embodiments, the AAV constructs may incorporate a sequence encoding the second-site nicking guide RNA. In still other embodiments, the AAV constructs may incorporate a sequence encoding the second-site nicking guide RNA and a sequence encoding the gRNA.

In various embodiments, the gRNAs and the second-site nicking guide RNAs can be expressed from an appropriate promoter, such as a human U6 (hU6) promoter, a mouse U6 (mU6) promoter, or other appropriate promoter. The gRNAs and the second-site nicking guide RNAs can be driven by the same promoters or different promoters.

In some embodiments, a rAAV constructs or the herein compositions are administered to a subject enterally. In some embodiments, a rAAV constructs or the herein compositions are administered to the subject parenterally. In some embodiments, a rAAV particle or the herein compositions are administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs. In some embodiments, a rAAV particle or the herein compositions are administered to the subject by injection into the hepatic artery or portal vein.

In other aspects, the base editors can be divided at a split site and provided as two halves of a whole/complete base editor. The two halves can be delivered to cells (e.g., as expressed proteins or on separate expression vectors) and once in contact inside the cell, the two halves form the complete base editor through the self-splicing action of the inteins on each base editor half. Split intein sequences can be engineered into each of the halves of the encoded base editor to facilitate their transplicing inside the cell and the concomitant restoration of the complete, functioning base editor.

These split intein-based methods overcome several barriers to in vivo delivery. For example, the DNA encoding base editors is larger than the rAAV packaging limit, and so requires special solutions. One such solution is formulating the editor fused to split intein pairs that are packaged into two separate rAAV particles that, when co-delivered to a cell, reconstitute the functional editor protein. Several other special considerations to account for the unique features of prime editing are described, including the optimization of second-site nicking targets and properly packaging base editors into virus vectors, including lentiviruses and rAAV.

In this aspect, the base editors can be divided at a split site and provided as two halves of a whole/complete base editor. The two halves can be delivered to cells (e.g., as expressed proteins or on separate expression vectors) and once in contact inside the cell, the two halves form the complete base editor through the self-splicing action of the inteins on each base editor half. Split intein sequences can be engineered into each of the halves of the encoded base editor to facilitate their transplicing inside the cell and the concomitant restoration of the complete, functioning base editor.

In various embodiments, the base editors may be engineered as two half proteins (i.e., a BE N-terminal half and a BE C-terminal half) by “splitting” the whole base editor as a “split site.” The “split site” refers to the location of insertion of split intein sequences (i.e., the N intein and the C intein) between two adjacent amino acid residues in the base editor. More specifically, the “split site” refers to the location of dividing the whole base editor into two separate halves, wherein in each halve is fused at the split site to either the N intein or the C intein motifs. The split site can be at any suitable location in the base editor fusion protein, but preferably the split site is located at a position that allows for the formation of two half proteins which are appropriately sized for delivery (e.g., by expression vector) and wherein the inteins, which are fused to each half protein at the split site termini, are available to sufficiently interact with one another when one half protein contacts the other half protein inside the cell.

In some embodiments, the split site is located in the napDNAbp domain. In other embodiments, the split site is located in the RT domain. In other embodiments, the split site is located in a linker that joins the napDNAbp domain and the RT domain.

In various embodiments, split site design requires finding sites to split and insert an N- and C-terminal intein that are both structurally permissive for purposes of packaging the two half base editor domains into two different AAV genomes. Additionally, intein residues necessary for trans splicing can be incorporated by mutating residues at the N terminus of the C terminal extein or inserting residues that will leave an intein “scar.”

In various embodiments, using SpCas9 nickase (SEQ ID NO: 59, 1368 amino acids) as an example, the split can between any two amino acids between 1 and 1368. Preferred splits, however, will be located between the central region of the protein, e.g., from amino acids 50-1250, or from 100-1200, or from 150-1150, or from 200-1100, or from 250-1050, or from 300-1000, or from 350-950, or from 400-900, or from 450-850, or from 500-800, or from 550-750, or from 600-700 of SEQ ID NO: 59. In specific exemplary embodiments, the split site may be between 740/741, or 801/802, or 1010/1011, or 1041/1042. In other embodiments the split site may be between 1/2, 2/3, 3/4, 4/5, 5/6, 6/7, 7/8, 8/9, 9/10, 10/11, 12/13, 14/15, 15/16, 17/18, 19/20 . . . 50/51 . . . 100/101 . . . 200/201 . . . 300/301 . . . 400/401 . . . 500/501 . . . 600/601 . . . 700/701 . . . 800/801 . . . 900/901 . . . 1000/1001 . . . 1100/1101 . . . 1200/1201 . . . 1300/1301 . . . and 1367/1368, including all adjacent pairs of amino acid residues.

In various embodiments, the split intein sequences can be engineered from the intein sequences represented by SEQ ID NOs: 17-24

In various embodiments, the split inteins can be used to separately deliver separate portions of a complete Base editor fusion protein to a cell, which upon expression in a cell, become reconstituted as a complete Base editor fusion protein through the trans splicing.

In some embodiments, the disclosure provides a method of delivering a Base editor fusion protein to a cell, comprising: constructing a first expression vector encoding an N-terminal fragment of the Base editor fusion protein fused to a first split intein sequence; constructing a second expression vector encoding a C-terminal fragment of the Base editor fusion protein fused to a second split intein sequence; delivering the first and second expression vectors to a cell, wherein the N-terminal and C-terminal fragment are reconstituted as the Base editor fusion protein in the cell as a result of trans splicing activity causing self-excision of the first and second split intein sequences.

In other embodiments, the split site is in the napDNAbp domain.

In still other embodiments, the split site is in the adenosine deaminase domain.

In yet other embodiments, the split site is in the linker.

In other embodiments, the base editors may be delivered by ribonucleoprotein complexes.

In this aspect, the base editors may be delivered by non-viral delivery strategies involving delivery of a base editor complexed with a gRNA (i.e., a BE ribonucleoprotein complex) by various methods, including electroporation and lipid nanoparticles. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

In some aspects, the invention provides methods comprising delivering one or more fusion proteins or polynucleotides encoding such fusion proteins, such as or one or more vectors as described herein encoding one or more components of the mtDNA editing system provided herein (e.g., deamination of mitochondrial DNA by a fusion protein or multiple fusion proteins) described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a base editor (e.g., deaminating enzyme) as described herein in combination with (and optionally complexed with) a guide domain (e.g., mitoTALE) is delivered to a cell. Conventional viral and non-viral based gene transfer methods can be used to introduce nucleic acids in mammalian cells or target tissues. Such methods can be used to administer nucleic acids encoding components of a base editor to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bihm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386; 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner: WO 91/17424 and WO 91/16024. Delivery can be to cells (e.g., in vitro or ex vivo administration) or target tissues (e.g., in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated, and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a viruses can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US 2003-0087817, incorporated herein by reference.

VII. gRNAs

In certain embodiments, the evolved DddA containing base editors may be fused to a napDNAbp, which are targeted by a corresponding guide RNA (gRNA) to a target deamination site.

Some aspects of the invention relate to guide sequences (“guide RNA” or “gRNA”) that are capable of guiding a napDNAbp or a base editor comprising a napDNAbp to a target site in a DNA molecule. In various embodiments base editors (e.g., base editors provided herein) can be complexed, bound, or otherwise associated with (e.g., via any type of covalent or non-covalent bond) one or more guide sequences, i.e., the sequence which becomes associated or bound to the base editor and directs its localization to a specific target sequence having complementarity to the guide sequence or a portion thereof. The particular design aspects of a guide sequence will depend upon the nucleotide sequence of a genomic target site of interest and the type of napDNA/RNAbp (e.g., type of Cas protein) present in the base editor, among other factors, such as PAM sequence locations, percent G/C content in the target sequence, the degree of microhomology regions, secondary structures, etc.

In embodiments relating Evolved DddA-containing base editors comprising Cas9/gRNA complexes, the Cas9 and gRNA components will need to be localized to the mitochondria. Cas9 can be modified with one or more MTS as discussed herein. In addition, the guide RNA may be localized to the mitochondria using known localization techniques for mRNA localization to mitochondria.

In general, a guide sequence is any polynucleotide sequence having sufficient complementarity with a target polynucleotide sequence to hybridize with the target sequence and direct sequence-specific binding of a napDNAbp (e.g., a Cas9, Cas9 homolog, or Cas9 variant) to the target sequence, such as a sequence within an SMN2 gene that comprises a C840T point mutation.

In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 31, 32, 33, 34, 35, 40, 45, 50, 75, or more nucleotides in length.

In some embodiments, a guide sequence is less than about 75, 50, 45, 40, 35, 30, 25, 20, 15, 12, or fewer nucleotides in length. The ability of a guide sequence to direct sequence-specific binding of a base editor to a target sequence may be assessed by any suitable assay. For example, the components of a base editor, including the guide sequence to be tested, may be provided to a host cell having the corresponding target sequence (e.g., a HGADFN 167 or HGADFN 188 cell line), such as by transfection with vectors encoding the components of a base editor disclosed herein, followed by an assessment of preferential cleavage within the target sequence, such as by Surveyor assay as described herein. Similarly, cleavage of a target polynucleotide sequence may be evaluated in a test tube by providing the target sequence, components of a base editor, including the guide sequence to be tested and a control guide sequence different from the test guide sequence, and comparing binding or rate of cleavage at the target sequence between the test and control guide sequence reactions. Other assays are possible, and will occur to those skilled in the art. The sequences of suitable guide RNAs for targeting Cas9:nucleic acid editing enzyme/domain fusion proteins to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting any of the provided fusion proteins to specific target sequences are provided herein. Additional guide sequences are well known in the art and can be used with the base editors described herein.

Additional exemplary guide sequences are disclosed in, for example, Jinek M., et al., Science 337:816-821(2012); Mali P, Esvelt K M & Church G M (2013) Cas9 as a versatile tool for engineering biology, Nature Methods, 10, 957-963; Li J F et al., (2013) Multiplex and homologous recombination-mediated genome editing in Arabidopsis and Nicotiana benthamiana using guide RNA and Cas9, Nature Biotechnology, 31, 688-691; Hwang, W. Y. et al., Efficient genome editing in zebrafish using a CRISPR-Cas system, Nature Biotechnology 31, 227-229 (2013); Cong L et al., (2013) Multiplex genome engineering using CRISPR/Cas systems, Science, 339, 819-823; Cho S W et al., (2013) Targeted genome engineering in human cells with the Cas9 RNA-guided endonuclease, Nature Biotechnology, 31, 230-232; Jinek, M. et al., RNA-programmed genome editing in human cells, eLife 2, e00471 (2013); Dicarlo, J. E. et al., Genome engineering in Saccharomyces cerevisiae using CRISPR-Cas systems. Nucleic Acid Res. (2013); Briner A E et al., (2014) Guide RNA functional modules direct Cas9 activity and orthogonality, Mol Cell, 56, 333-339, the entire contents of each of which are herein incorporated by reference.

Some aspects of this disclosure provide methods of making the evolved base editors disclosed herein, or base editor complexes comprising one or more napR/DNAbp-programming nucleic acid molecules (e.g., Cas9 guide RNAs) and a nucleobase editor provided herein. In addition, some aspects of the disclosure provide methods of using the evolved base editors for editing a target nucleotide sequence (e.g., a genome).

### Continuous Evolution Methods

Various aspects of the disclosure relate to providing continuous evolution methods and systems (e.g., appropriate vectors, cells, phage, flow vessels, etc.).

The continuous evolution methods provided herein allow for a gene of interest (e.g., a base editor gene) in a viral vector to be evolved over multiple generations of viral life cycles in a flow of host cells to acquire a desired function or activity.

Some aspects of this invention provide a method of continuous evolution of a gene of interest, comprising (a) contacting a population of host cells with a population of viral vectors comprising the gene of interest, wherein (1) the host cell is amenable to infection by the viral vector; (2) the host cell expresses viral genes required for the generation of viral particles; (3) the expression of at least one viral gene required for the production of an infectious viral particle is dependent on a function of the gene of interest; and (4) the viral vector allows for expression of the protein in the host cell, and can be replicated and packaged into a viral particle by the host cell. In some embodiments, the method comprises (b) contacting the host cells with a mutagen. In some embodiments, the method further comprises (c) incubating the population of host cells under conditions allowing for viral replication and the production of viral particles, wherein host cells are removed from the host cell population, and fresh, uninfected host cells are introduced into the population of host cells, thus replenishing the population of host cells and creating a flow of host cells. The cells are incubated in all embodiments under conditions allowing for the gene of interest to acquire a mutation. In some embodiments, the method further comprises (d) isolating a mutated version of the viral vector, encoding an evolved gene product (e.g., protein), from the population of host cells.

In some embodiments, a method of phage-assisted continuous evolution is provided comprising (a) contacting a population of bacterial host cells with a population of phages that comprise a gene of interest to be evolved and that are deficient in a gene required for the generation of infectious phage, wherein (1) the phage allows for expression of the gene of interest in the host cells; (2) the host cells are suitable host cells for phage infection, replication, and packaging; and (3) the host cells comprise an expression construct encoding the gene required for the generation of infectious phage, wherein expression of the gene is dependent on a function of a gene product of the gene of interest. In some embodiments the method further comprises (b) incubating the population of host cells under conditions allowing for the mutation of the gene of interest, the production of infectious phage, and the infection of host cells with phage, wherein infected cells are removed from the population of host cells, and wherein the population of host cells is replenished with fresh host cells that have not been infected by the phage. In some embodiments, the method further comprises (c) isolating a mutated phage replication product encoding an evolved protein from the population of host cells.

In some embodiments, the viral vector or the phage is a filamentous phage, for example, an M13 phage, such as an M13 selection phage as described in more detail elsewhere herein. In some such embodiments, the gene required for the production of infectious viral particles is the M13 gene III (gIII).

In some embodiments, the viral vector infects mammalian cells. In some embodiments, the viral vector is a retroviral vector. In some embodiments, the viral vector is a vesicular stomatitis virus (VSV) vector. As a dsRNA virus, VSV has a high mutation rate, and can carry cargo, including a gene of interest, of up to 4.5 kb in length. The generation of infectious VSV particles requires the envelope protein VSV-G, a viral glycoprotein that mediates phosphatidylserine attachment and cell entry. VSV can infect a broad spectrum of host cells, including mammalian and insect cells. VSV is therefore a highly suitable vector for continuous evolution in human, mouse, or insect host cells. Similarly, other retroviral vectors that can be pseudotyped with VSV-G envelope protein are equally suitable for continuous evolution processes as described herein.

It is known to those of skill in the art that many retroviral vectors, for example, Murine Leukemia Virus vectors, or Lentiviral vectors can efficiently be packaged with VSV-G envelope protein as a substitute for the virus's native envelope protein. In some embodiments, such VSV-G packagable vectors are adapted for use in a continuous evolution system in that the native envelope (env) protein (e.g., VSV-G in VSVS vectors, or env in MLV vectors) is deleted from the viral genome, and a gene of interest is inserted into the viral genome under the control of a promoter that is active in the desired host cells. The host cells, in turn, express the VSV-G protein, another env protein suitable for vector pseudotyping, or the viral vector's native env protein, under the control of a promoter the activity of which is dependent on an activity of a product encoded by the gene of interest, so that a viral vector with a mutation leading to an increased activity of the gene of interest will be packaged with higher efficiency than a vector with baseline or a loss-of-function mutation.

In some embodiments, mammalian host cells are subjected to infection by a continuously evolving population of viral vectors, for example, VSV vectors comprising a gene of interest and lacking the VSV-G encoding gene, wherein the host cells comprise a gene encoding the VSV-G protein under the control of a conditional promoter. Such retrovirus-bases system could be a two-vector system (the viral vector and an expression construct comprising a gene encoding the envelope protein), or, alternatively, a helper virus can be employed, for example, a VSV helper virus. A helper virus typically comprises a truncated viral genome deficient of structural elements required to package the genome into viral particles, but including viral genes encoding proteins required for viral genome processing in the host cell, and for the generation of viral particles. In such embodiments, the viral vector-based system could be a three-vector system (the viral vector, the expression construct comprising the envelope protein driven by a conditional promoter, and the helper virus comprising viral functions required for viral genome propagation but not the envelope protein). In some embodiments, expression of the five genes of the VSV genome from a helper virus or expression construct in the host cells, allows for production of infectious viral particles carrying a gene of interest, indicating that unbalanced gene expression permits viral replication at a reduced rate, suggesting that reduced expression of VSV-G would indeed serve as a limiting step in efficient viral production.

One advantage of using a helper virus is that the viral vector can be deficient in genes encoding proteins or other functions provided by the helper virus, and can, accordingly, carry a longer gene of interest. In some embodiments, the helper virus does not express an envelope protein, because expression of a viral envelope protein is known to reduce the infectability of host cells by some viral vectors via receptor interference. Viral vectors, for example retroviral vectors, suitable for continuous evolution processes, their respective envelope proteins, and helper viruses for such vectors, are well known to those of skill in the art. For an overview of some exemplary viral genomes, helper viruses, host cells, and envelope proteins suitable for continuous evolution procedures as described herein, see Coffin et al., Retroviruses, CSHL Press 1997, ISBN0-87969-571-4, incorporated herein in its entirety.

In some embodiments, the incubating of the host cells is for a time sufficient for at least 10, at least 20, at least 30, at least 40, at least 50, at least 100, at least 200, at least 300, at least 400, at least, 500, at least 600, at least 700, at least 800, at least 900, at least 1000, at least 1250, at least 1500, at least 1750, at least 2000, at least 2500, at least 3000, at least 4000, at least 5000, at least 7500, at least 10000, or more consecutive viral life cycles. In certain embodiments, the viral vector is an M13 phage, and the length of a single viral life cycle is about 10-20 minutes.

In some embodiments, the cells are contacted and/or incubated in suspension culture. For example, in some embodiments, bacterial cells are incubated in suspension culture in liquid culture media. Suitable culture media for bacterial suspension culture will be apparent to those of skill in the art, and the invention is not limited in this regard. See, for example, Molecular Cloning: A Laboratory Manual, 2nd Ed., ed. by Sambrook, Fritsch, and Maniatis (Cold Spring Harbor Laboratory Press: 1989); Elizabeth Kutter and Alexander Sulakvelidze: Bacteriophages: Biology and Applications. CRC Press; 1st edition (December 2004), ISBN: 0849313368; Martha R. J. Clokie and Andrew M. Kropinski: Bacteriophages: Methods and Protocols, Volume 1: Isolation, Characterization, and Interactions (Methods in Molecular Biology) Humana Press; 1st edition (December, 2008), ISBN: 1588296822; Martha R. J. Clokie and Andrew M. Kropinski: Bacteriophages: Methods and Protocols, Volume 2: Molecular and Applied Aspects (Methods in Molecular Biology) Humana Press; 1st edition (December 2008), ISBN: 1603275649; all of which are incorporated herein in their entirety by reference for disclosure of suitable culture media for bacterial host cell culture).Suspension culture typically requires the culture media to be agitated, either continuously or intermittently. This is achieved, in some embodiments, by agitating or stirring the vessel comprising the host cell population. In some embodiments, the outflow of host cells and the inflow of fresh host cells is sufficient to maintain the host cells in suspension. This in particular, if the flow rate of cells into and/or out of the lagoon is high.

In some embodiments, a viral vector/host cell combination is chosen in which the life cycle of the viral vector is significantly shorter than the average time between cell divisions of the host cell. Average cell division times and viral vector life cycle times are well known in the art for many cell types and vectors, allowing those of skill in the art to ascertain such host cell/vector combinations. In certain embodiments, host cells are being removed from the population of host cells contacted with the viral vector at a rate that results in the average time of a host cell remaining in the host cell population before being removed to be shorter than the average time between cell divisions of the host cells, but to be longer than the average life cycle of the viral vector employed. The result of this is that the host cells, on average, do not have sufficient time to proliferate during their time in the host cell population while the viral vectors do have sufficient time to infect a host cell, replicate in the host cell, and generate new viral particles during the time a host cell remains in the cell population. This assures that the only replicating nucleic acid in the host cell population is the viral vector, and that the host cell genome, the accessory plasmid, or any other nucleic acid constructs cannot acquire mutations allowing for escape from the selective pressure imposed.

For example, in some embodiments, the average time a host cell remains in the host cell population is about 10, about 11, about 12, about 13, about 14, about 15, about 16, about 17, about 18, about 19, about 20, about 21, about 22, about 23, about 24, about 25, about 30, about 35, about 40, about 45, about 50, about 55, about 60, about 70, about 80, about 90, about 100, about 120, about 150, or about 180 minutes.

In some embodiments, the average time a host cell remains in the host cell population depends on how fast the host cells divide and how long infection (or conjugation) requires. In general, the flow rate should be faster than the average time required for cell division, but slow enough to allow viral (or conjugative) propagation. The former will vary, for example, with the media type, and can be delayed by adding cell division inhibitor antibiotics (FtsZ inhibitors in E. coli, etc.). Since the limiting step in continuous evolution is production of the protein required for gene transfer from cell to cell, the flow rate at which the vector washes out will depend on the current activity of the gene(s) of interest. In some embodiments, titratable production of the protein required for the generation of infectious particles, as described herein, can mitigate this problem. In some embodiments, an indicator of phage infection allows computer-controlled optimization of the flow rate for the current activity level in real-time.

In some embodiments, the host cell population is continuously replenished with fresh, uninfected host cells. In some embodiments, this is accomplished by a steady stream of fresh host cells into the population of host cells. In other embodiments, however, the inflow of fresh host cells into the lagoon is semi-continuous or intermittent (e.g., batch-fed). In some embodiments, the rate of fresh host cell inflow into the cell population is such that the rate of removal of cells from the host cell population is compensated. In some embodiments, the result of this cell flow compensation is that the number of cells in the cell population is substantially constant over the time of the continuous evolution procedure. In some embodiments, the portion of fresh, uninfected cells in the cell population is substantially constant over the time of the continuous evolution procedure. For example, in some embodiments, about 10%, about 15%, about 20%, about 25%, about 30%, about 40%, about 50%, about 60%, about 70%, about 75%, about 80%, or about 90% of the cells in the host cell population are not infected by virus. In general, the faster the flow rate of host cells is, the smaller the portion of cells in the host cell population that are infected will be. However, faster flow rates allow for more transfer cycles, e.g., viral life cycles, and, thus, for more generations of evolved vectors in a given period of time, while slower flow rates result in a larger portion of infected host cells in the host cell population and therefore a larger library size at the cost of slower evolution. In some embodiments, the range of effective flow rates is invariably bounded by the cell division time on the slow end and vector washout on the high end In some embodiments, the viral load, for example, as measured in infectious viral particles per volume of cell culture media is substantially constant over the time of the continuous evolution procedure.

In some embodiments, the fresh host cells comprise the accessory plasmid required for selection of viral vectors, for example, the accessory plasmid comprising the gene required for the generation of infectious phage particles that is lacking from the phages being evolved. In some embodiments, the host cells are generated by contacting an uninfected host cell with the relevant vectors, for example, the accessory plasmid and, optionally, a mutagenesis plasmid, and growing an amount of host cells sufficient for the replenishment of the host cell population in a continuous evolution experiment. Methods for the introduction of plasmids and other gene constructs into host cells are well known to those of skill in the art and the invention is not limited in this respect. For bacterial host cells, such methods include, but are not limited to electroporation and heat-shock of competent cells. In some embodiments, the accessory plasmid comprises a selection marker, for example, an antibiotic resistance marker, and the fresh host cells are grown in the presence of the respective antibiotic to ensure the presence of the plasmid in the host cells. Where multiple plasmids are present, different markers are typically used. Such selection markers and their use in cell culture are known to those of skill in the art, and the invention is not limited in this respect.

In some embodiments, the host cell population in a continuous evolution experiment is replenished with fresh host cells growing in a parallel, continuous culture. In some embodiments, the cell density of the host cells in the host cell population contacted with the viral vector and the density of the fresh host cell population is substantially the same.

Typically, the cells being removed from the cell population contacted with the viral vector comprise cells that are infected with the viral vector and uninfected cells. In some embodiments, cells are being removed from the cell populations continuously, for example, by effecting a continuous outflow of the cells from the population. In other embodiments, cells are removed semi-continuously or intermittently from the population. In some embodiments, the replenishment of fresh cells will match the mode of removal of cells from the cell population, for example, if cells are continuously removed, fresh cells will be continuously introduced. However, in some embodiments, the modes of replenishment and removal may be mismatched, for example, a cell population may be continuously replenished with fresh cells, and cells may be removed semi-continuously or in batches.

In some embodiments, the rate of fresh host cell replenishment and/or the rate of host cell removal is adjusted based on quantifying the host cells in the cell population. For example, in some embodiments, the turbidity of culture media comprising the host cell population is monitored and, if the turbidity falls below a threshold level, the ratio of host cell inflow to host cell outflow is adjusted to effect an increase in the number of host cells in the population, as manifested by increased cell culture turbidity. In other embodiments, if the turbidity rises above a threshold level, the ratio of host cell inflow to host cell outflow is adjusted to effect a decrease in the number of host cells in the population, as manifested by decreased cell culture turbidity. Maintaining the density of host cells in the host cell population within a specific density range ensures that enough host cells are available as hosts for the evolving viral vector population, and avoids the depletion of nutrients at the cost of viral packaging and the accumulation of cell-originated toxins from overcrowding the culture.

In some embodiments, the cell density in the host cell population and/or the fresh host cell density in the inflow is about 102 cells/ml to about 1012 cells/ml. In some embodiments, the host cell density is about 102 cells/ml, about 103 cells/ml, about 104 cells/ml, about 105 cells/ml, about 5.105 cells/ml, about 106 cells/ml, about 5-106 cells/ml, about 107 cells/ml, about 5-107 cells/ml, about 108 cells/ml, about 5.108 cells/ml, about 109 cells/ml, about 5.109 cells/ml, about 1010 cells/ml, or about 5.1010 cells/ml. In some embodiments, the host cell density is more than about 1010 cells/ml.

In some embodiments, the host cell population is contacted with a mutagen. In some embodiments, the cell population contacted with the viral vector (e.g., the phage), is continuously exposed to the mutagen at a concentration that allows for an increased mutation rate of the gene of interest, but is not significantly toxic for the host cells during their exposure to the mutagen while in the host cell population. In other embodiments, the host cell population is contacted with the mutagen intermittently, creating phases of increased mutagenesis, and accordingly, of increased viral vector diversification. For example, in some embodiments, the host cells are exposed to a concentration of mutagen sufficient to generate an increased rate of mutagenesis in the gene of interest for about 10%, about 20%, about 50%, or about 75% of the time.

In some embodiments, the host cells comprise a mutagenesis expression construct, for example, in the case of bacterial host cells, a mutagenesis plasmid. In some embodiments, the mutagenesis plasmid comprises a gene expression cassette encoding a mutagenesis-promoting gene product, for example, a proofreading-impaired DNA polymerase. In other embodiments, the mutagenesis plasmid, including a gene involved in the SOS stress response, (e.g., UmuC, UmuD′, and/or RecA). In some embodiments, the mutagenesis-promoting gene is under the control of an inducible promoter. Suitable inducible promoters are well known to those of skill in the art and include, for example, arabinose-inducible promoters, tetracycline or doxycyclin-inducible promoters, and tamoxifen-inducible promoters. In some embodiments, the host cell population is contacted with an inducer of the inducible promoter in an amount sufficient to effect an increased rate of mutagenesis. For example, in some embodiments, a bacterial host cell population is provided in which the host cells comprise a mutagenesis plasmid in which a dnaQ926, UmuC, UmuD′, and RecA expression cassette is controlled by an arabinose-inducible promoter. In some such embodiments, the population of host cells is contacted with the inducer, for example, arabinose in an amount sufficient to induce an increased rate of mutation.

The use of an inducible mutagenesis plasmid allows one to generate a population of fresh, uninfected host cells in the absence of the inducer, thus avoiding an increased rate of mutation in the fresh host cells before they are introduced into the population of cells contacted with the viral vector.

Once introduced into this population, however, these cells can then be induced to support an increased rate of mutation, which is particularly useful in some embodiments of continuous evolution. For example, in some embodiments, the host cell comprise a mutagenesis plasmid as described herein, comprising an arabinose-inducible promoter driving expression of dnaQ926, UmuC, UmuD′, and RecA730 from a pBAD promoter (see, e.g., Khlebnikov A, Skaug T, Keasling JD. Modulation of gene expression from the arabinose-inducible araBAD promoter. J Ind Microbiol Biotechnol. 2002 July; 29(1):34-7; incorporated herein by reference for disclosure of a pBAD promoter). In some embodiments, the fresh host cells are not exposed to arabinose, which activates expression of the above identified genes and, thus, increases the rate of mutations in the arabinose-exposed cells, until the host cells reach the lagoon in which the population of selection phage replicates. Accordingly, in some embodiments, the mutation rate in the host cells is normal until they become part of the host cell population in the lagoon, where they are exposed to the inducer (e.g., arabinose) and, thus, to increased mutagenesis. In some embodiments, a method of continuous evolution is provided that includes a phase of diversifying the population of viral vectors by mutagenesis, in which the cells are incubated under conditions suitable for mutagenesis of the viral vector in the absence of stringent selection for the mutated replication product of the viral vector encoding the evolved protein. This is particularly useful in embodiments in which a desired function to be evolved is not merely an increase in an already present function, for example, an increase in the transcriptional activation rate of a transcription factor, but the acquisition of a function not present in the gene of interest at the outset of the evolution procedure. A step of diversifying the pool of mutated versions of the gene of interest within the population of viral vectors, for example, of phage, allows for an increase in the chance to find a mutation that conveys the desired function.

In some embodiments, diversifying the viral vector population is achieved by providing a flow of host cells that does not select for gain-of-function mutations in the gene of interest for replication, mutagenesis, and propagation of the population of viral vectors. In some embodiments, the host cells are host cells that express all genes required for the generation of infectious viral particles, for example, bacterial cells that express a complete helper phage, and, thus, do not impose selective pressure on the gene of interest. In other embodiments, the host cells comprise an accessory plasmid comprising a conditional promoter with a baseline activity sufficient to support viral vector propagation even in the absence of significant gain-of-function mutations of the gene of interest. This can be achieved by using a “leaky” conditional promoter, by using a high-copy number accessory plasmid, thus amplifying baseline leakiness, and/or by using a conditional promoter on which the initial version of the gene of interest effects a low level of activity while a desired gain-of-function mutation effects a significantly higher activity.

For example, as described in more detail in the Example section, in some embodiments, a population of host cells comprising a high-copy accessory plasmid with a gene required for the generation of infectious phage particles is contacted with a selection phage comprising a gene of interest, wherein the accessory plasmid comprises a conditional promoter driving expression of the gene required for the generation from a conditional promoter, the activity of which is dependent on the activity of a gene product encoded by the gene of interest. In some such embodiments, a low stringency selection phase can be achieved by designing the conditional promoter in a way that the initial gene of interest exhibits some activity on that promoter. For example, if a transcriptional activator, such as a T7RNAP or a transcription factor is to be evolved to recognize a non-native target DNA sequence (e.g., a T3RNAP promoter sequence, on which T7RNAP has no activity), a low-stringency accessory plasmid can be designed to comprise a conditional promoter in which the target sequence comprises a desired characteristic, but also retains a feature of the native recognition sequence that allows the transcriptional activator to recognize the target sequence, albeit with less efficiency than its native target sequence. Initial exposure to such a low-stringency accessory plasmid comprising a hybrid target sequence (e.g., a T7/T3 hybrid promoter, with some features of the ultimately desired target sequence and some of the native target sequence) allows the population of phage vectors to diversify by acquiring a plurality of mutations that are not immediately selected against based on the permissive character of the accessory plasmid. Such a diversified population of phage vectors can then be exposed to a stringent selection accessory plasmid, for example, a plasmid comprising in its conditional promoter the ultimately desired target sequence that does not retain a feature of the native target sequence, thus generating a strong negative selective pressure against phage vectors that have not acquired a mutation allowing for recognition of the desired target sequence.

In some embodiments, an initial host cell population contacted with a population of evolving viral vectors is replenished with fresh host cells that are different from the host cells in the initial population. For example, in some embodiments, the initial host cell population is made of host cells comprising a low-stringency accessory plasmid, or no such plasmid at all, or are permissible for viral infection and propagation. In some embodiments, after diversifying the population of viral vectors in the low-stringency or no-selection host cell population, fresh host cells are introduced into the host cell population that impose a more stringent selective pressure for the desired function of the gene of interest. For example, in some embodiments, the secondary fresh host cells are not permissible for viral replication and propagation anymore. In some embodiments, the stringently selective host cells comprise an accessory plasmid in which the conditional promoter exhibits none or only minimal baseline activity, and/or which is only present in low or very low copy numbers in the host cells.

Such methods involving host cells of varying selective stringency allow for harnessing the power of continuous evolution methods as provided herein for the evolution of functions that are completely absent in the initial version of the gene of interest, for example, for the evolution of a transcription factor recognizing a foreign target sequence that a native transcription factor, used as the initial gene of interest, does not recognize at all. Or, for another example, the recognition of a desired target sequence by a DNA-binding protein, a recombinase, a nuclease, a zinc-finger protein, or an RNA-polymerase, that does not bind to or does not exhibit any activity directed towards the desired target sequence.

In some embodiments, negative selection is applied during a continuous evolution method as described herein, by penalizing undesired activities. In some embodiments, this is achieved by causing the undesired activity to interfere with pIII production. For example, expression of an antisense RNA complementary to the gIII RBS and/or start codon is one way of applying negative selection, while expressing a protease (e.g., TEV) and engineering the protease recognition sites into pIII is another.

In some embodiments, negative selection is applied during a continuous evolution method as described herein, by penalizing the undesired activities of evolved products. This is useful, for example, if the desired evolved product is an enzyme with high specificity, for example, a transcription factor or protease with altered, but not broadened, specificity. In some embodiments, negative selection of an undesired activity is achieved by causing the undesired activity to interfere with pIII production, thus inhibiting the propagation of phage genomes encoding gene products with an undesired activity. In some embodiments, expression of a dominant-negative version of pIII or expression of an antisense RNA complementary to the gIII RBS and/or gIII start codon is linked to the presence of an undesired activity. In some embodiments, a nuclease or protease cleavage site, the recognition or cleavage of which is undesired, is inserted into a pIII transcript sequence or a pIII amino acid sequence, respectively. In some embodiments, a transcriptional or translational repressor is used that represses expression of a dominant negative variant of pIII and comprises a protease cleavage site the recognition or cleavage of which is undesired.

In some embodiments, counter-selection against activity on non-target substrates is achieved by linking undesired evolved product activities to the inhibition of phage propagation. For example, in some embodiments, in which a transcription factor is evolved to recognize a specific target sequence, but not an undesired off-target sequence, a negative selection cassette is employed, comprising a nucleic acid sequence encoding a dominant-negative version of pIII (pIII-neg) under the control of a promoter comprising the off-target sequence. If an evolution product recognizes the off-target sequence, the resulting phage particles will incorporate pIII-neg, which results in an inhibition of phage infective potency and phage propagation, thus constituting a selective disadvantage for any phage genomes encoding an evolution product exhibiting the undesired, off-target activity, as compared to evolved products not exhibiting such an activity. In some embodiments, a dual selection strategy is applied during a continuous evolution experiment, in which both positive selection and negative selection constructs are present in the host cells. In some such embodiments, the positive and negative selection constructs are situated on the same plasmid, also referred to as a dual selection accessory plasmid.

For example, in some embodiments, a dual selection accessory plasmid is employed comprising a positive selection cassette, comprising a pIII-encoding sequence under the control of a promoter comprising a target nucleic acid sequence, and a negative selection cassette, comprising a pIII-neg encoding cassette under the control of a promoter comprising an off-target nucleic acid sequence. One advantage of using a simultaneous dual selection strategy is that the selection stringency can be fine-tuned based on the activity or expression level of the negative selection construct as compared to the positive selection construct. Another advantage of a dual selection strategy is the selection is not dependent on the presence or the absence of a desired or an undesired activity, but on the ratio of desired and undesired activities, and, thus, the resulting ratio of pIII and pIII-neg that is incorporated into the respective phage particle.

Some aspects of this invention provide or utilize a dominant negative variant of pIII (pIII-neg). These aspects are based on the surprising discovery that a pIII variant that comprises the two N-terminal domains of pIII and a truncated, termination-incompetent C-terminal domain is not only inactive but is a dominant-negative variant of pIII. A pIII variant comprising the two N-terminal domains of pIII and a truncated, termination-incompetent C-terminal domain was described in Bennett, N. J.; Rakonjac, J., Unlocking of the filamentous bacteriophage virion during infection is mediated by the C domain of pII. Journal of Molecular Biology 2006, 356 (2), 266-73; the entire contents of which are incorporated herein by reference. However, the dominant negative property of such pIII variants has not been previously described. Some aspects of this invention are based on the surprising discovery that a pIII-neg variant as provided herein is efficiently incorporated into phage particles, but it does not catalyze the unlocking of the particle for entry during infection, rendering the respective phage noninfectious even if wild type pIII is present in the same phage particle. Accordingly, such pIII-neg variants are useful for devising a negative selection strategy in the context of PACE, for example, by providing an expression construct comprising a nucleic acid sequence encoding a pIII-neg variant under the control of a promoter comprising a recognition motif, the recognition of which is undesired. In other embodiments, pIII-neg is used in a positive selection strategy, for example, by providing an expression construct in which a pIII-neg encoding sequence is controlled by a promoter comprising a nuclease target site or a repressor recognition site, the recognition of either one is desired.

Positive and negative selection strategies can further be designed to link non-DNA directed activities to phage propagation efficiency. For example, protease activity towards a desired target protease cleavage site can be linked to pIII expression by devising a repressor of gene expression that can be inactivated by a protease recognizing the target site. In some embodiments, pIII expression is driven by a promoter comprising a binding site for such a repressor. Suitable transcriptional repressors are known to those in the art, and one exemplary repressor is the lambda repressor protein, that efficiently represses the lambda promoter pR and can be modified to include a desired protease cleavage site (see, e.g., Sices, H. J.; Kristie, T. M., A genetic screen for the isolation and characterization of site-specific proteases. Proc Natl Acad Sci USA 1998, 95 (6), 2828-33; and Sices, H. J.; Leusink, M. D.; Pacheco, A.; Kristie, T. M., Rapid genetic selection of inhibitor-resistant protease mutants: clinically relevant and novel mutants of the HIV protease. AIDS Res Hum Retroviruses 2001, 17 (13), 1249-55, the entire contents of each of which are incorporated herein by reference). The lambda repressor (cI) contains an N-terminal DNA binding domain and a C-terminal dimerization domain. These two domains are connected by a flexible linker. Efficient transcriptional repression requires the dimerization of cI, and, thus, cleavage of the linker connecting dimerization and binding domains results in abolishing the repressor activity of cI.

Some embodiments provide a pIII expression construct that comprises a pR promoter (containing cI binding sites) driving expression of pIII. When expressed together with a modified cI comprising a desired protease cleavage site in the linker sequence connecting dimerization and binding domains, the cI molecules will repress pIII transcription in the absence of the desired protease activity, and this repression will be abolished in the presence of such activity, thus providing a linkage between protease cleavage activity and an increase in pIII expression that is useful for positive PACE protease selection. Some embodiments provide a negative selection strategy against undesired protease activity in PACE evolution products. In some embodiments, the negative selection is conferred by an expression cassette comprising a pIII-neg encoding nucleic acid under the control of a cI-repressed promoter. When co-expressed with a cI repressor protein comprising an undesired protease cleavage site, expression of pIII-neg will occur in cell harboring phage expressing a protease exhibiting protease activity towards the undesired target site, thus negatively selecting against phage encoding such undesired evolved products. A dual selection for protease target specificity can be achieved by co-expressing cI-repressible pIII and pIII-neg encoding expression constructs with orthogonal cI variants recognizing different DNA target sequences, and thus allowing for simultaneous expression without interfering with each other. Orthogonal cI variants in both dimerization specificity and DNA-binding specificity are known to those of skill in the art (see, e.g., Wharton, R. P.; Ptashne, M., Changing the binding specificity of a repressor by redesigning an alphahelix. Nature 1985, 316 (6029), 601-5; and Wharton, R. P.; Ptashne, M., A new-specificity mutant of 434 repressor that defines an amino acid-base pair contact. Nature 1987, 326 (6116), 888-91, the entire contents of each of which are incorporated herein by reference).

Other selection schemes for gene products having a desired activity are well known to those of skill in the art or will be apparent from the instant disclosure. Selection strategies that can be used in continuous evolution processes and methods as provided herein include, but are not limited to, selection strategies useful in two-hybrid screens. For example, the T7 RNAP selection strategy described in more detail elsewhere herein is an example of a promoter recognition selection strategy. Two-hybrid accessory plasmid setups further permit the evolution of protein-protein interactions, and accessory plasmids requiring site-specific recombinase activity for production of the protein required for the generation of infectious viral particles, for example, pIII, allow recombinases to be evolved to recognize any desired target site. A two-hybrid setup or a related one-hybrid setup can further be used to evolve DNA-binding proteins, while a three-hybrid setup can evolve RNA-protein interactions.

Biosynthetic pathways producing small molecules can also be evolved with a promoter or riboswitch (e.g., controlling gene III expression/translation) that is responsive to the presence of the desired small molecule. For example, a promoter that is transcribed only in the presence of butanol could be placed on the accessory plasmid upstream of gene III to optimize a biosynthetic pathway encoding the enzymes for butanol synthesis. A phage vector carrying a gene of interest that has acquired an activity boosting butanol synthesis would have a selective advantage over other phages in an evolving phage population that have not acquired such a gain-of-function. Alternatively, a chemical complementation system, for example, as described in Baker and Cornish, PNAS, 2002, incorporated herein by reference, can be used to evolve individual proteins or enzymes capable of bond formation reactions ( ). In other embodiments, a trans-splicing intron designed to splice itself into a particular target sequence can be evolved by expressing only the latter half of gene III from the accessory plasmid, preceded by the target sequence, and placing the other half (fused to the trans-splicing intron) on the selection phage. Successful splicing would reconstitute full-length pIII-encoding mRNA. Protease specificity and activity can be evolved by expressing pIII fused to a large protein from the accessory plasmid, separated by a linker containing the desired protease recognition site. Cleavage of the linker by active protease encoded by the selection phage would result in infectious pIII, while uncleaved pIII would be unable to bind due to the blocking protein. Further, As described, for example, by Malmborg and Borrebaeck 1997, a target antigen can be fused to the F pilus of a bacteria, blocking wild-type pIII from binding. Phage displaying antibodies specific to the antigen could bind and infect, yielding enrichments of >1000-fold in phage display. In some embodiments, this system can be adapted for continuous evolution, in that the accessory plasmid is designed to produce wild-type pIII to contact the tolA receptor and perform the actual infection (as the antibody-pIII fusion binds well but infects with low efficiency), while the selection phage encodes the pIII-antibody fusion protein. Progeny phage containing both types of pIII tightly adsorb to the F pilus through the antibody-antigen interaction, with the wild-type pIII contacting tolA and mediating high-efficiency infection. To allow propagation when the initial antibody-antigen interaction is weak, a mixture of host cells could flow into the lagoon: a small fraction expressing wild-type pili and serving as a reservoir of infected cells capable of propagating any selection phage regardless of activity, while the majority of cells requires a successful interaction, serving as the “reward” for any mutants that improve their binding affinity. This last system, in some embodiments, can evolve new antibodies that are effective against a target pathogen faster than the pathogen itself can evolve, since the evolution rates of PACE and other systems described herein are higher than those of human-specific pathogens, for example, those of human viruses.

Methods and strategies to design conditional promoters suitable for carrying out the selections strategies described herein are well known to those of skill in the art. Some exemplary design strategies are summarized in FIG. 3B. For an overview over exemplary suitable selection strategies and methods for designing conditional promoters driving the expression of a gene required for cell-cell gene transfer, e.g. gIII, see Vidal and Legrain, Yeast n-hybrid review, Nucleic Acid Research 27, 919 (1999), incorporated herein in its entirety.

### Apparatus for Continued Evolution

The invention also provides apparatuses for continuous evolution of a nucleic acid. The core element of such an apparatus is a lagoon allowing for the generation of a flow of host cells in which a population of viral vectors can replicate and propagate. In some embodiments, the lagoon comprises a cell culture vessel comprising an actively replicating population of viral vectors, for example, phage vectors comprising a gene of interest, and a population of host cells, for example, bacterial host cells. In some embodiments, the lagoon comprises an inflow for the introduction of fresh host cells into the lagoon and an outflow for the removal of host cells from the lagoon. In some embodiments, the inflow is connected to a turbidostat comprising a culture of fresh host cells. In some embodiments, the outflow is connected to a waste vessel, or a sink. In some embodiments, the lagoon further comprises an inflow for the introduction of a mutagen into the lagoon. In some embodiments that inflow is connected to a vessel holding a solution of the mutagen. In some embodiments, the lagoon comprises an inflow for the introduction of an inducer of gene expression into the lagoon, for example, of an inducer activating an inducible promoter within the host cells that drives expression of a gene promoting mutagenesis (e.g., as part of a mutagenesis plasmid), as described in more detail elsewhere herein. In some embodiments, that inflow is connected to a vessel comprising a solution of the inducer, for example, a solution of arabinose.

In some embodiments, the lagoon comprises a population of viral vectors. In some embodiments, the lagoon comprises a population of viral vectors. In some embodiments, the viral vectors are phage, for example, M13 phages deficient in a gene required for the generation of infectious viral particles as described herein. In some such embodiments, the host cells are prokaryotic cells amenable to phage infection, replication, and propagation of phage, for example, host cells comprising an accessory plasmid comprising a gene required for the generation of infectious viral particles under the control of a conditional promoter as described herein.

In some embodiments, the lagoon comprises a controller for regulation of the inflow and outflow rates of the host cells, the inflow of the mutagen, and/or the inflow of the inducer. In some embodiments, a visual indicator of phage presence, for example, a fluorescent marker, is tracked and used to govern the flow rate, keeping the total infected population constant. In some embodiments, the visual marker is a fluorescent protein encoded by the phage genome, or an enzyme encoded by the phage genome that, once expressed in the host cells, results in a visually detectable change in the host cells. In some embodiments, the visual tracking of infected cells is used to adjust a flow rate to keep the system flowing as fast as possible without risk of vector washout.

In some embodiments, the expression of the gene required for the generation of infectious particles is titratable. In some embodiments, this is accomplished with an accessory plasmid producing pIII proportional to the amount of anhydrotetracycline added to the lagoon. Other In some embodiments, such a titrable expression construct can be combined with another accessory plasmid as described herein, allowing simultaneous selection for activity and titratable control of pIII. This permits the evolution of activities too weak to otherwise survive in the lagoon, as well as allowing neutral drift to escape local fitness peak traps. In some embodiments, negative selection is applied during a continuous evolution method as described herein, by penalizing undesired activities.

In some embodiments, this is achieved by causing the undesired activity to interfere with pIII production. For example, expression of an antisense RNA complementary to the gIII RBS and/or start codon is one way of applying negative selection, while expressing a protease (e.g., TEV) and engineering the protease recognition sites into pIII is another.

In some embodiments, the apparatus comprises a turbidostat. In some embodiments, the turbidostat comprises a cell culture vessel in which the population of fresh host cells is situated, for example, in liquid suspension culture. In some embodiments, the turbidostat comprises an outflow that is connected to an inflow of the lagoon, allowing the introduction of fresh cells from the turbidostat into the lagoon. In some embodiments, the turbidostat comprises an inflow for the introduction of fresh culture media into the turbidostat. In some embodiments, the inflow is connected to a vessel comprising sterile culture media. In some embodiments, the turbidostat further comprises an outflow for the removal of host cells from the turbidostat. In some embodiments, that outflow is connected to a waste vessel or drain.

In some embodiments, the turbidostat comprises a turbidity meter for measuring the turbidity of the culture of fresh host cells in the turbidostat. In some embodiments, the turbidostat comprises a controller that regulated the inflow of sterile liquid media and the outflow into the waste vessel based on the turbidity of the culture liquid in the turbidostat.

In some embodiments, the lagoon and/or the turbidostat comprises a shaker or agitator for constant or intermittent agitation, for example, a shaker, mixer, stirrer, or bubbler, allowing for the population of host cells to be continuously or intermittently agitated and oxygenated.

In some embodiments, the controller regulates the rate of inflow of fresh host cells into the lagoon to be substantially the same (volume/volume) as the rate of outflow from the lagoon. In some embodiments, the rate of inflow of fresh host cells into and/or the rate of outflow of host cells from the lagoon is regulated to be substantially constant over the time of a continuous evolution experiment. In some embodiments, the rate of inflow and/or the rate of outflow is from about 0.1 lagoon volumes per hour to about 25 lagoon volumes per hour. In some embodiments, the rate of inflow and/or the rate of outflow is approximately 0.1 lagoon volumes per hour (lv/h), approximately 0.2 lv/h, approximately 0.25 lv/h, approximately 0.3 lv/h, approximately 0.4 lv/h, approximately 0.5 lv/h, approximately 0.6 lv/h, approximately 0.7 lv/h, approximately 0.75 lv/h, approximately 0.8 lv/h, approximately 0.9 lv/h, approximately 1 lv/h, approximately 2 lv/h, approximately 2.5 lv/h, approximately 3 lv/h, approximately 4 lv/h, approximately 5 lv/h, approximately 7.5 lv/h, approximately 10 lv/h, or more than 10 lv/h.

In some embodiments, the inflow and outflow rates are controlled based on a quantitative assessment of the population of host cells in the lagoon, for example, by measuring the cell number, cell density, wet biomass weight per volume, turbidity, or cell growth rate. In some embodiments, the lagoon inflow and/or outflow rate is controlled to maintain a host cell density of from about 102 cells/ml to about 1012 cells/ml in the lagoon. In some embodiments, the inflow and/or outflow rate is controlled to maintain a host cell density of about 102 cells/ml, about 103 cells/ml, about 104 cells/ml, about 105 cells/ml, about 5×105 cells/ml, about 106 cells/ml, about 5×106 cells/ml, about 107 cells/ml, about 5×107 cells/ml, about 108 cells/ml, about 5×108 cells/ml, about 109 cells/ml, about 5×109 cells/ml, about 1010 cells/ml, about 5×1010 cells/ml, or more than 5×1010 cells/ml, in the lagoon. In some embodiments, the density of fresh host cells in the turbidostat and the density of host cells in the lagoon are substantially identical.

In some embodiments, the lagoon inflow and outflow rates are controlled to maintain a substantially constant number of host cells in the lagoon. In some embodiments, the inflow and outflow rates are controlled to maintain a substantially constant frequency of fresh host cells in the lagoon. In some embodiments, the population of host cells is continuously replenished with fresh host cells that are not infected by the phage. In some embodiments, the replenishment is semi-continuous or by batch-feeding fresh cells into the cell population.

In some embodiments, the lagoon volume is from approximately 1 ml to approximately 100 l, for example, the lagoon volume is approximately 1 ml, approximately 10 ml, approximately 50 ml, approximately 100 ml, approximately 200 ml, approximately 250 ml, approximately 500 ml, approximately 750 ml, approximately 11, approximately 2 ml, approximately 2.51, approximately 31, approximately 4 l, approximately 5 l, approximately 10 l, approximately 1 ml-10 ml, approximately 10m-50 ml, approximately 50 ml-100, approximately 100 ml-250 ml, approximately 250 ml-500 ml, approximately 500 ml-1 l, approximately 1 l-2 l, approximately 2 l-5 l, approximately 51-10 l, approximately 10-50 l, approximately 50-100 l, or more than 100 l.

In some embodiments, the lagoon and/or the turbidostat further comprises a heater and a thermostat controlling the temperature. In some embodiments, the temperature in the lagoon and/or the turbidostat is controlled to be from about 4° C. to about 55° C., preferably from about 25° C. to about 39° C., for example, about 37° C.

In some embodiments, the inflow rate and/or the outflow rate is controlled to allow for the incubation and replenishment of the population of host cells for a time sufficient for at least 10, at least 20, at least 30, at least 40, at least 50, at least 100, at least 200, at least 300, at least 400, at least, 500, at least 600, at least 700, at least 800, at least 900, at least 1000, at least 1250, at least 1500, at least 1750, at least 2000, at least 2500, at least 3000, at least 4000, at least 5000, at least 7500, at least 10000, or more consecutive viral vector or phage life cycles. In some embodiments, the time sufficient for one phage life cycle is about 10 minutes.

Therefore, in some embodiments, the time of the entire evolution procedure is about 12 hours, about 18 hours, about 24 hours, about 36 hours, about 48 hours, about 50 hours, about 3 days, about 4 days, about 5 days, about 6 days, about 7 days, about 10 days, about two weeks, about 3 weeks, about 4 weeks, or about 5 weeks.

For example, in some embodiments, a PACE apparatus is provided, comprising a lagoon of about 100 ml, or about 1 l volume, wherein the lagoon is connected to a turbidostat of about 0.5 l, 1 l, or 3 l volume, and to a vessel comprising an inducer for a mutagenesis plasmid, for example, arabinose, wherein the lagoon and the turbidostat comprise a suspension culture of E. coli cells at a concentration of about 5×108 cells/ml. In some embodiments, the flow of cells through the lagoon is regulated to about 3 lagoon volumes per hour. In some embodiments, cells are removed from the lagoon by continuous pumping, for example, by using a waste needle set at a height of the lagoon vessel that corresponds to a desired volume of fluid (e.g., about 100 ml, in the lagoon. In some embodiments, the host cells are E. coli cells comprising the F′ plasmid, for example, cells of the genotype F′proA+B+Δ(lacIZY) zzf::Tn10(TetR)/endA1 recA1 galE15 galK16 nupG rpsL ΔlacIZYA araD139 Δ(ara,leu)7697 mcrA Δ(mrr-hsdRMS-mcrBC) proBA::pir116 λ−. In some embodiments, the selection phage comprises an M13 genome, in which the pIII-encoding region, or a part thereof, has been replaced with a gene of interest, for example, a coding region that is driven by a wild-type phage promoter. In some embodiments, the host cells comprise an accessory plasmid in which a gene encoding a protein required for the generation of infectious phage particles, for example, M13 pIII, is expressed from a conditional promoter as described in more detail elsewhere herein. In some embodiments, the host cells further comprise a mutagenesis plasmid, for example, a mutagenesis plasmid expressing a mutagenesis-promoting protein from an inducible promoter, such as an arabinose-inducible promoter. In some embodiments the apparatus is set up to provide fresh media to the turbidostat for the generation of a flow of cells of about 2-4 lagoon volumes per hour for about 3-7 days.

### Vectors and Reagents

The invention provides viral vectors for the inventive continuous evolution processes. In some embodiments, phage vectors for phage-assisted continuous evolution are provided. In some embodiments, a selection phage is provided that comprises a phage genome deficient in at least one gene required for the generation of infectious phage particles and a gene of interest to be evolved.

For example, in some embodiments, the selection phage comprises an M13 phage genome deficient in a gene required for the generation of infectious M13 phage particles, for example, a full-length gIII. In some embodiments, the selection phage comprises a phage genome providing all other phage functions required for the phage life cycle except the gene required for generation of infectious phage particles. In some such embodiments, an M13 selection phage is provided that comprises a gI, gII, gIV, gV, gVI, gVII, gVIII, gIX, and a gX gene, but not a full-length gIIL. In some embodiments, the selection phage comprises a 3′-fragment of gIII, but no full-length gIIL. The 3′-end of gIII comprises a promoter (see FIG. 16) and retaining this promoter activity is beneficial, in some embodiments, for an increased expression of gVI, which is immediately downstream of the gIII 3′-promoter, or a more balanced (wild-type phage-like) ratio of expression levels of the phage genes in the host cell, which, in turn, can lead to more efficient phage production. In some embodiments, the 3′-fragment of gIII gene comprises the 3′-gIII promoter sequence. In some embodiments, the 3′-fragment of gIII comprises the last 180 bp, the last 150 bp, the last 125 bp, the last 100 bp, the last 50 bp, or the last 25 bp of gII. In some embodiments, the 3′-fragment of gIII comprises the last 180 bp of gIII.

M13 selection phage is provided that comprises a gene of interest in the phage genome, for example, inserted downstream of the gVIII 3′-terminator and upstream of the gIII-3′-promoter. In some embodiments, an M13 selection phage is provided that comprises a multiple cloning site for cloning a gene of interest into the phage genome, for example, a multiple cloning site (MCS) inserted downstream of the gVIII 3′-terminator and upstream of the gIII-3′-promoter.

Some aspects of this invention provide a vector system for continuous evolution procedures, comprising of a viral vector, for example, a selection phage, and a matching accessory plasmid. In some embodiments, a vector system for phage-based continuous directed evolution is provided that comprises (a) a selection phage comprising a gene of interest to be evolved, wherein the phage genome is deficient in a gene required to generate infectious phage; and (b) an accessory plasmid comprising the gene required to generate infectious phage particle under the control of a conditional promoter, wherein the conditional promoter is activated by a function of a gene product encoded by the gene of interest.

In some embodiments, the selection phage is an M13 phage as described herein. For example, in some embodiments, the selection phage comprises an M13 genome including all genes required for the generation of phage particles, for example, gI, gII, gIV, gV, gVI, gVII, gVIII, gIX, and gX gene, but not a full-length gIII gene. In some embodiments, the selection phage genome comprises an F1 or an M13 origin of replication. In some embodiments, the selection phage genome comprises a 3′-fragment of gIII gene. In some embodiments, the selection phage comprises a multiple cloning site upstream of the gIII 3′-promoter and downstream of the gVIII 3′-terminator.

In some embodiments, the selection phage does not comprise a full length gVI. GVI is similarly required for infection as gIII and, thus, can be used in a similar fashion for selection as described for gIII herein. However, it was found that continuous expression of pIII renders some host cells resistant to infection by M13. Accordingly, it is desirable that pIII is produced only after infection. This can be achieved by providing a gene encoding pIII under the control of an inducible promoter, for example, an arabinose-inducible promoter as described herein, and providing the inducer in the lagoon, where infection takes place, but not in the turbidostat, or otherwise before infection takes place. In some embodiments, multiple genes required for the generation of infectious phage are removed from the selection phage genome, for example, gIII and gVI, and provided by the host cell, for example, in an accessory plasmid as described herein.

The vector system may further comprise a helper phage, wherein the selection phage does not comprise all genes required for the generation of phage particles, and wherein the helper phage complements the genome of the selection phage, so that the helper phage genome and the selection phage genome together comprise at least one functional copy of all genes required for the generation of phage particles, but are deficient in at least one gene required for the generation of infectious phage particles.

In some embodiments, the accessory plasmid of the vector system comprises an expression cassette comprising the gene required for the generation of infectious phage under the control of a conditional promoter. In some embodiments, the accessory plasmid of the vector system comprises a gene encoding pIII under the control of a conditional promoter the activity of which is dependent on a function of a product of the gene of interest.

In some embodiments, the vector system further comprises a mutagenesis plasmid, for example, an arabinose-inducible mutagenesis plasmid as described herein.

In some embodiments, the vector system further comprises a helper plasmid providing expression constructs of any phage gene not comprised in the phage genome of the selection phage or in the accessory plasmid.

In various embodiments of the vectors used herein in the continuous evolution processes may include the following components in any combination:

gRNA backbone
(SEQ ID NO: 199)
gttttagagctagaaatagcaagttaaaataaggctagtccgttatcaac
ttgaaaaagtggcaccgagtcggtgcttttttt
T7 RNA Polymerase
(SEQ ID NO: 346)
MNTINIAKNDFSDIELAAIPFNTLADHYGERLAREQLALEHESYEMGEAR
FRKMFERQLKAGEVADNAAAKPLITTLLPKMIARINDWFEEVKAKRGKRP
TAFQFLQEIKPEAVAYITIKTTLACLTSADNTTVQAVASAIGRAIEDEAR
FGRIRDLKAKHFKKNVEEQLNKRVGHVYKKAFMQVVEADMLSKGLLGGEA
WSSWHKEDSIHVGVRCIEMLIESTGMVSLHRQNAGVVGQDSETIELAPEY
AEAIATRAGALAGISPMFQPCVVPPKPWTGITGGGYWANGRRPLALVRTH
SKKALMRYEDVYMPEVYKAINIAQNTAWKINKKVLAVANVITKWKHCPVE
DIPAIEREELPMKPEDIDMNPEALTAWKRAAAAVYRKDKARKSRRISLEF
MLEQANKFANHKAIWFPYNMDWRGRVYAVSMFNPQGNDMTKGLLTLAKGK
PIGKEGYYWLKIHGANCAGVDKVPFPERIKFIEENHENIMACAKSPLENT
WWAEQDSPFCFLAFCFEYAGVQHHGLSYNCSLPLAFDGSCSGIQHFSAML
RDEVGGRAVNLLPSETVQDIYGIVAKKVNEILQADAINGTDNEVVTVTDE
NTGEISEKVKLGTKALAGQWLAYGVTRSVTKRSVMTLAYGSKEFGFRQQV
LEDTIQPAIDSGKGLMFTQPNQAAGYMAKLIWESVSVTVVAAVEAMNWLK
SAAKLLAAEVKDKKTGEILRKRCAVHWVTPDGFPVWQEYKKPIQTRLNLM
FLGQFRLQPTINTNKDSEIDAHKQESGIAPNFVHSQDGSHLRKTVVWAHE
KYGIESFALIHDSFGTIPADAANLFKAVRETMVDTYESCDVLADFYDQFN
LNLRDIADQLHESQLDKMPALPAKGLESDFAFA*
Degron tag
(SEQ ID NO: 348)
AANDENYNYALAA
Fusion sequence
(SEQ ID NO: 196)
MNTINIAKNDFSDIELAAIPFNTLADHYGERLAREQLALEHESYEMGEAR
FRKMFERQLKAGEVADNAAAKPLITTLLPKMIARINDWFEEVKAKRGKRP
TAFQFLQEIKPEAVAYITIKTTLACLTSADNTTVQAVASAIGRAIEDEAR
FGRIRDLKAKHFKKNVEEQLNKRVGHVYKKAFMQVVEADMLSKGLLGGEA
WSSWHKEDSIHVGVRCIEMLIESTGMVSLHRQNAGVVGQDSETIELAPEY
AEAIATRAGALAGISPMFQPCVVPPKPWTGITGGGYWANGRRPLALVRTH
SKKALMRYEDVYMPEVYKAINIAQNTAWKINKKVLAVANVITKWKHCPVE
DIPAIEREELPMKPEDIDMNPEALTAWKRAAAAVYRKDKARKSRRISLEF
MLEQANKFANHKAIWFPYNMDWRGRVYAVSMFNPQGNDMTKGLLTLAKGK
PIGKEGYYWLKIHGANCAGVDKVPFPERIKFIEENHENIMACAKSPLENT
WWAEQDSPFCFLAFCFEYAGVQHHGLSYNCSLPLAFDGSCSGIQHFSAML
RDEVGGRAVNLLPSETVQDIYGIVAKKVNEILQADAINGTDNEVVTVTDE
NTGEISEKVKLGTKALAGQWLAYGVTRSVTKRSVMTLAYGSKEFGFRQQV
LEDTIQPAIDSGKGLMFTQPNQAAGYMAKLIWESVSVTVVAAVEAMNWLK
SAAKLLAAEVKDKKTGEILRKRCAVHWVTPDGFPVWQEYKKPIQTRLNLM
FLGQFRLQPTINTNKDSEIDAHKQESGIAPNFVHSQDGSHLRKTVVWAHE
KYGIESFALIHDSFGTIPADAANLFKAVRETMVDTYESCDVLADFYDQFA
DQLHESQLDKMPALPAKGNLNLRDILESDFAFAWTRAANDENYNYALAA*
DnaE intein (fusion to deaminases via the XTEN
linker)
(SEQ ID NO: 200)
CLSYETEILTVEYGLLPIGKIVEKRIECTVYSVDNNGNIYTQPVAQWHDR
GEQEVFEYCLEDGSLIRATKDHKFMTVDGQMLPIDEIFERELDLMRVDNL
PN*
Fusion to APOBEC
(SEQ ID NO: 197)
MSSETGPVAVDPTLRRRIEPHEFEVFFDPRELRKETCLLYEINWGGRHSI
WRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESG
YCWRNFVNYSPSNEAHWPRYPHLWVRLYVLELYCIILGLPPCLNILRRKQ
PQLTFFTIALQSCHYQRLPPHILWATGLKSGSETPGTSESATPECLSYET
EILTVEYGLLPIGKIVEKRIECTVYSVDNNGNIYTQPVAQWHDRGEQEVF
EYCLEDGSLIRATKDHKFMTVDGQMLPIDEIFERELDLMRVDNLPN*
C-intein fused to cas9
(SEQ ID NO: 201)
MIKIATRKYLGKQNVYDIGVERDHNFALKNGFIASNC
Fusion to cas9
(SEQ ID NO: 198)
MIKIATRKYLGKQNVYDIGVERDHNFALKNGFIASNCFNKYSIGLAIGTN
SVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKF
RGHFLIEGDLNPDNSDVDKLFIQLVQTYNQLFEENPINASGVDAKAILSA
RLSKSRRLENLIAQLPGEKKNGLFGNLIALSLGLTPNFKSNFDLAEDAKL
QLSKDTYDDDLDNLLAQIGDQYADLFLAAKNLSDAILLSDILRVNTEITK
APLSASMIKRYDEHHQDLTLLKALVRQQLPEKYKEIFFDQSKNGYAGYID
GGASQEEFYKFIKPILEKMDGTEELLVKLNREDLLRKQRTFDNGSIPHQI
HLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLL
YEYFTVYNELTKVKYVTEGMRKPAFLSGEQKKAIVDLLFKTNRKVTVKQL
KEDYFKKIECFDSVEISGVEDRFNASLGTYHDLLKIIKDKDFLDNEENED
ILEDIVLTLTLFEDREMIEERLKTYAHLFDDKVMKQLKRRRYTGWGRLSR
KLINGIRDKQSGKTILDFLKSDGFANRNFMQLIHDDSLTFKEDIQKAQVS
GQGDSLHEHIANLAGSPAIKKGILQTVKVVDELVKVMGRHKPENIVIEMA
RENQTTQKGQKNSRERMKRIEEGIKELGSQILKEHPVENTQLQNEKLYLY
YLQNGRDMYVDQELDINRLSDYDVDAIVPQSFLKDDSIDNKVLTRSDKNR
GKSDNVPSEEVVKKMKNYWRQLLNAKLITQRKFDNLTKAERGGLSELDKA
GFIKRQLVETRQITKHVAQILDSRMNTKYDENDKLIREVKVITLKSKLVS
DFRKDFQFYKVREINNYHHAHDAYLNAVVGTALIKKYPKLESEFVYGDYK
VYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQ
LGGDSGGSMTNLSDIIEKETGKQLVIQESILMLPEEVEEVIGNKPESDIL
VHTAYDESTDENVMLLTSDAPEYKPWALVIQDSNGENKIKML*

### VIII. Method of Continuous Evolution

Various aspects of the disclosure relate to providing directed evolution methods and systems (e.g., appropriate vectors, cells, phage, flow vessels, etc.) for making the evolved DddA variants described herein.

The directed evolution methods provided herein allow for a gene of interest (e.g., gene or sequence encoding a starter DddA protein described herein) in a viral vector to be evolved over multiple generations of viral life cycles in a flow of host cells to acquire a desired function or activity, i.e., an improved DddA variant with higher deaminase activity and/or broader sequence context.

Some aspects of this invention provide a method of continuous evolution of a gene of interest, comprising (a) contacting a population of host cells with a population of viral vectors comprising the gene of interest, wherein (1) the host cell is amenable to infection by the viral vector; (2) the host cell expresses viral genes required for the generation of viral particles; (3) the expression of at least one viral gene required for the production of an infectious viral particle is dependent on a function of the gene of interest; and (4) the viral vector allows for expression of the protein in the host cell, and can be replicated and packaged into a viral particle by the host cell. In some embodiments, the method comprises (b) contacting the host cells with a mutagen. In some embodiments, the method further comprises (c) incubating the population of host cells under conditions allowing for viral replication and the production of viral particles, wherein host cells are removed from the host cell population, and fresh, uninfected host cells are introduced into the population of host cells, thus replenishing the population of host cells and creating a flow of host cells. The cells are incubated in all embodiments under conditions allowing for the gene of interest to acquire a mutation. In some embodiments, the method further comprises (d) isolating a mutated version of the viral vector, encoding an evolved gene product (e.g., protein), from the population of host cells.

In some embodiments, a method of phage-assisted continuous evolution is provided comprising (a) contacting a population of bacterial host cells with a population of phages that comprise a gene of interest to be evolved and that are deficient in a gene required for the generation of infectious phage, wherein (1) the phage allows for expression of the gene of interest in the host cells; (2) the host cells are suitable host cells for phage infection, replication, and packaging; and (3) the host cells comprise an expression construct encoding the gene required for the generation of infectious phage, wherein expression of the gene is dependent on a function of a gene product of the gene of interest. In some embodiments the method further comprises (b) incubating the population of host cells under conditions allowing for the mutation of the gene of interest, the production of infectious phage, and the infection of host cells with phage, wherein infected cells are removed from the population of host cells, and wherein the population of host cells is replenished with fresh host cells that have not been infected by the phage. In some embodiments, the method further comprises (c) isolating a mutated phage replication product encoding an evolved protein from the population of host cells.

In some embodiments, the viral vector or the phage is a filamentous phage, for example, an M13 phage, such as an M13 selection phage as described in more detail elsewhere herein. In some such embodiments, the gene required for the production of infectious viral particles is the M13 gene III (gIII).

In some embodiments, the viral vector infects mammalian cells. In some embodiments, the viral vector is a retroviral vector. In some embodiments, the viral vector is a vesicular stomatitis virus (VSV) vector. As a dsRNA virus, VSV has a high mutation rate, and can carry cargo, including a gene of interest, of up to 4.5 kb in length. The generation of infectious VSV particles requires the envelope protein VSV-G, a viral glycoprotein that mediates phosphatidylserine attachment and cell entry. VSV can infect a broad spectrum of host cells, including mammalian and insect cells. VSV is therefore a highly suitable vector for continuous evolution in human, mouse, or insect host cells. Similarly, other retroviral vectors that can be pseudotyped with VSV-G envelope protein are equally suitable for continuous evolution processes as described herein.

It is known to those of skill in the art that many retroviral vectors, for example, Murine Leukemia Virus vectors, or Lentiviral vectors can efficiently be packaged with VSV-G envelope protein as a substitute for the virus's native envelope protein. In some embodiments, such VSV-G packagable vectors are adapted for use in a continuous evolution system in that the native envelope (env) protein (e.g., VSV-G in VSVS vectors, or env in MLV vectors) is deleted from the viral genome, and a gene of interest is inserted into the viral genome under the control of a promoter that is active in the desired host cells. The host cells, in turn, express the VSV-G protein, another env protein suitable for vector pseudotyping, or the viral vector's native env protein, under the control of a promoter the activity of which is dependent on an activity of a product encoded by the gene of interest, so that a viral vector with a mutation leadin G to T increased activity of the gene of interest will be packaged with higher efficiency than a vector with baseline or a loss-of-function mutation.

In some embodiments, mammalian host cells are subjected to infection by a continuously evolving population of viral vectors, for example, VSV vectors comprising a gene of interest and lacking the VSV-G encoding gene, wherein the host cells comprise a gene encoding the VSV-G protein under the control of a conditional promoter. Such retrovirus-bases system could be a two-vector system (the viral vector and an expression construct comprising a gene encoding the envelope protein), or, alternatively, a helper virus can be employed, for example, a VSV helper virus. A helper virus typically comprises a truncated viral genome deficient of structural elements required to package the genome into viral particles, but including viral genes encoding proteins required for viral genome processing in the host cell, and for the generation of viral particles. In such embodiments, the viral vector-based system could be a three-vector system (the viral vector, the expression construct comprising the envelope protein driven by a conditional promoter, and the helper virus comprising viral functions required for viral genome propagation but not the envelope protein). In some embodiments, expression of the five genes of the VSV genome from a helper virus or expression construct in the host cells, allows for production of infectious viral particles carrying a gene of interest, indicating that unbalanced gene expression permits viral replication at a reduced rate, suggesting that reduced expression of VSV-G would indeed serve as a limiting step in efficient viral production.

One advantage of using a helper virus is that the viral vector can be deficient in genes encoding proteins or other functions provided by the helper virus, and can, accordingly, carry a longer gene of interest. In some embodiments, the helper virus does not express an envelope protein, because expression of a viral envelope protein is known to reduce the infectability of host cells by some viral vectors via receptor interference. Viral vectors, for example retroviral vectors, suitable for continuous evolution processes, their respective envelope proteins, and helper viruses for such vectors, are well known to those of skill in the art. For an overview of some exemplary viral genomes, helper viruses, host cells, and envelope proteins suitable for continuous evolution procedures as described herein, see Coffin et al., Retroviruses, CSHL Press 1997, ISBN0-87969-571-4, incorporated herein in its entirety.

In some embodiments, the incubating of the host cells is for a time sufficient for at least 10, at least 20, at least 30, at least 40, at least 50, at least 100, at least 200, at least 300, at least 400, at least, 500, at least 600, at least 700, at least 800, at least 900, at least 1000, at least 1250, at least 1500, at least 1750, at least 2000, at least 2500, at least 3000, at least 4000, at least 5000, at least 7500, at least 10000, or more consecutive viral life cycles. In certain embodiments, the viral vector is an M13 phage, and the length of a single viral life cycle is about 10-20 minutes.

In some embodiments, the cells are contacted and/or incubated in suspension culture. For example, in some embodiments, bacterial cells are incubated in suspension culture in liquid culture media. Suitable culture media for bacterial suspension culture will be apparent to those of skill in the art, and the invention is not limited in this regard. See, for example, Molecular Cloning: A Laboratory Manual, 2nd Ed., ed. by Sambrook, Fritsch, and Maniatis (Cold Spring Harbor Laboratory Press: 1989); Elizabeth Kutter and Alexander Sulakvelidze: Bacteriophages: Biology and Applications. CRC Press; 1st edition (December 2004), ISBN: 0849313368; Martha R. J. Clokie and Andrew M. Kropinski: Bacteriophages: Methods and Protocols, Volume 1: Isolation, Characterization, and Interactions (Methods in Molecular Biology) Humana Press; 1st edition (December, 2008), ISBN: 1588296822; Martha R. J. Clokie and Andrew M. Kropinski: Bacteriophages: Methods and Protocols, Volume 2: Molecular and Applied Aspects (Methods in Molecular Biology) Humana Press; 1st edition (December 2008), ISBN: 1603275649; all of which are incorporated herein in their entirety by reference for disclosure of suitable culture media for bacterial host cell culture). Suspension culture typically requires the culture media to be agitated, either continuously or intermittently. This is achieved, in some embodiments, by agitating or stirring the vessel comprising the host cell population. In some embodiments, the outflow of host cells and the inflow of fresh host cells is sufficient to maintain the host cells in suspension. This in particular, if the flow rate of cells into and/or out of the lagoon is high.

In some embodiments, a viral vector/host cell combination is chosen in which the life cycle of the viral vector is significantly shorter than the average time between cell divisions of the host cell. Average cell division times and viral vector life cycle times are well known in the art for many cell types and vectors, allowing those of skill in the art to ascertain such host cell/vector combinations. In certain embodiments, host cells are being removed from the population of host cells contacted with the viral vector at a rate that results in the average time of a host cell remaining in the host cell population before being removed to be shorter than the average time between cell divisions of the host cells, but to be longer than the average life cycle of the viral vector employed. The result of this is that the host cells, on average, do not have sufficient time to proliferate during their time in the host cell population while the viral vectors do have sufficient time to infect a host cell, replicate in the host cell, and generate new viral particles during the time a host cell remains in the cell population. This assures that the only replicating nucleic acid in the host cell population is the viral vector, and that the host cell genome, the accessory plasmid, or any other nucleic acid constructs cannot acquire mutations allowing for escape from the selective pressure imposed.

For example, in some embodiments, the average time a host cell remains in the host cell population is about 10, about 11, about 12, about 13, about 14, about 15, about 16, about 17, about 18, about 19, about 20, about 21, about 22, about 23, about 24, about 25, about 30, about 35, about 40, about 45, about 50, about 55, about 60, about 70, about 80, about 90, about 100, about 120, about 150, or about 180 minutes.

In some embodiments, the average time a host cell remains in the host cell population depends on how fast the host cells divide and how long infection (or conjugation) requires. In general, the flow rate should be faster than the average time required for cell division, but slow enough to allow viral (or conjugative) propagation. The former will vary, for example, with the media type, and can be delayed by adding cell division inhibitor antibiotics (FtsZ inhibitors in E. coli, etc.). Since the limiting step in continuous evolution is production of the protein required for gene transfer from cell to cell, the flow rate at which the vector washes out will depend on the current activity of the gene(s) of interest. In some embodiments, titratable production of the protein required for the generation of infectious particles, as described herein, can mitigate this problem. In some embodiments, an indicator of phage infection allows computer-controlled optimization of the flow rate for the current activity level in real-time.

In various embodiments, the continuous evolution process is PACE, which is described in Thuronyi, B. W. et al. Nat Biotechnol 37, 1070-1079 (2019), the contents of which are incorporated herein by reference in their entirety. The general concept of PACE technology has also been described, for example, in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Application, U.S. Pat. No. 9,023,594, issued May 5, 2015, International PCT Application, PCT/US2015/012022, filed Jan. 20, 2015, published as WO 2015/134121 on Sep. 11, 2015, and International PCT Application, PCT/US2016/027795, filed Apr. 15, 2016, published as WO 2016/168631 on Oct. 20, 2016, the entire contents of each of which are incorporated herein by reference. PACE can be used, for instance, to evolve a deaminase (e.g., a cytidine or adenosine deaminase) which uses single strand DNA as a substrate to obtain a deaminase which is capable of using double-strand DNA as a substrate (e.g., DddA).

### IX. Methods of Treatment

The evolved DddA-containing base editors may be used to deaminate a target base in a double stranded DNA substrate.

The instant disclosure provides methods for the treatment of a subject diagnosed with a disease associated with or caused by a point mutation that can be corrected by the mtDNA editing system provided herein (e.g., deamination of mitochondrial DNA by a fusion protein or multiple fusion proteins). For example, in some embodiments, a method is provided that comprises administering to a subject having such a disease (e.g., MELAS/Leigh syndrome and Leber's hereditary optic neuropathy, other disorders associated with a point mutation as described above), an effective amount of the mtDNA editing system provided herein (e.g., deamination of mitochondrial DNA by a fusion protein or multiple fusion proteins) described herein that corrects the point mutation or introduces a point mutation comprising desired genetic change. In some embodiments, a method is provided that comprises administering to a subject having such a disease, (e.g., MELAS/Leigh syndrome and Leber's hereditary optic neuropathy, other disorders associated with a point mutation as described above), an effective amount of the mtDNA editing system provided herein (e.g., deamination of mitochondrial DNA by a fusion protein or multiple fusion proteins) described herein that corrects the point mutation or introduces a deactivating mutation into a disease-associated gene. In some embodiments, the disease is a proliferative disease. In some embodiments, the disease is a genetic disease. In some embodiments, the disease is a mitochondrial disease. In some embodiments, the disease is a metabolic disease. In some embodiments, the disease is a lysosomal storage disease. Other diseases that can be treated by correcting a point mutation or introducing a deactivating mutation into a disease-associated gene will be known to those of skill in the art, and the disclosure is not limited in this respect.

The instant disclosure provides methods for the treatment of additional diseases or disorders (e.g., diseases or disorders that are associated with or caused by a point mutation that can be corrected by the mtDNA editing system provided herein (e.g., deamination of mitochondrial DNA by a fusion protein or multiple fusion proteins) provided herein). Some such diseases are described herein, and additional suitable diseases that can be treated with the strategies and fusion proteins, or nucleic acids thereof, provided herein will be apparent to those of skill in the art based on the instant disclosure. Exemplary suitable diseases and disorders are listed below. It will be understood that the numbering of the specific positions or residues in the respective sequences depends on the particular protein and numbering scheme used. Numbering might be different (e.g., in precursors of a mature protein and the mature protein itself), and differences in sequences from species to species may affect numbering. One of skill in the art will be able to identify the respective residue in any homologous protein and in the respective encoding nucleic acid by methods well known in the art (e.g., by sequence alignment and determination of homologous residues). Exemplary suitable diseases and disorders include, without limitation: MELAS/Leigh syndrome and Leber's hereditary optic neuropathy.

The Evolved DddA-containing base editors described herein may be used to treat any mitochondrial disease or disorder. As used herein, “mitochondrial disorders” related to disorders which are due to abnormal mitochondria such as for example, a mitochondrial genetic mutation, enzyme pathways etc. Examples of disorders include and are not limited to: loss of motor control, muscle weakness and pain, gastro-intestinal disorders and swallowing difficulties, poor growth, cardiac disease, liver disease, diabetes, respiratory complications, seizures, visual/hearing problems, lactic acidosis, developmental delays and susceptibility to infection.

The mitochondrial abnormalities give rise to “mitochondrial diseases” which include, but not limited to: AD: Alzheimer's Disease; ADPD: Alzheimer's Disease and Parkinsons's Disease; AMDF: Ataxia, Myoclonus and Deafness CIPO: Chronic Intestinal Pseudoobstruction with myopathy and Opthalmoplegia; CPEO: Chronic Progressive External Opthalmoplegia; DEAF: Maternally inherited DEAFness or aminoglycoside-induced DEAFness; DEMCHO: Dementia and Chorea; DMDF: Diabetes Mellitus & DeaFness; Exercise Intolerance; ESOC: Epilepsy, Strokes, Optic atrophy, & Cognitive decline; FBSN: Familial Bilateral Striatal Necrosis; FICP: Fatal Infantile Cardiomyopathy Plus, a MELAS-associated cardiomyopathy; GER: Gastrointestinal Reflux; KSS Kearns Sayre Syndrome LDYT: Leber's hereditary optic neuropathy and DYsTonia; LHON: Leber Hereditary Optic Neuropathy; LFMM: Lethal Infantile Mitochondrial Myopathy; MDM: Myopathy and Diabetes Mellitus; MELAS:

Mitochondrial Encephalomyopathy, Lactic Acidosis, and Stroke-like episodes; MEPR: Myoclonic Epilepsy and Psychomotor Regression; MERME: MERRF/MELAS overlap disease; MERRF: Myoclonic Epilepsy and Ragged Red Muscle Fibers; MHCM: Maternally Inherited Hypertrophic CardioMyopathy; MICM: Maternally Inherited Cardiomyopathy; MILS: Maternally Inherited Leigh Syndrome; Mitochondrial Encephalocardiomyopathy; Mitochondrial Encephalomyopathy; MM: Mitochondrial Myopathy; MMC: Maternal Myopathy and Cardiomyopathy; Multisystem Mitochondrial Disorder (myopathy, encephalopathy, blindness, hearing loss, peripheral neuropathy); NARP: Neurogenic muscle weakness, Ataxia, and Retinitis Pigmentosa; alternate phenotype at this locus is reported as Leigh Disease; NIDDM: Non-Insulin Dependent Diabetes Mellitus; PEM: Progressive Encephalopathy; PME: Progressive Myoclonus Epilepsy; RTT: Rett Syndrome; SIDS: Sudden Infant Death Syndrome.

In embodiments, a mitochondrial disorder that may be treatable using the Evolved DddA-containing base editors described herein include Myoclonic Epilepsy with Ragged Red Fibers (MERRF); Mitochondrial Myopathy, Encephalopathy, Lactacidosis, and Stroke (MELAS); Maternally Inherited Diabetes and Deafness (MIDD); Leber's Hereditary Optic Neuropathy (LHON); chronic progressive external ophthalmoplegia (CPEO); Leigh Disease; Kearns-Sayre Syndrome (KSS); Friedreich's Ataxia (FRDA); Co-Enzyme QIO (CoQIO) Deficiency; Complex I Deficiency; Complex II Deficiency; Complex III Deficiency; Complex IV Deficiency; Complex V Deficiency; other myopathies; cardiomyopathy; encephalomyopathy; renal tubular acidosis; neurodegenerative diseases; Parkinson's disease; Alzheimer's disease; amyotrophic lateral sclerosis (ALS); motor neuron diseases; hearing and balance impairments; or other neurological disorders; epilepsy; genetic diseases; Huntington's Disease; mood disorders; nucleoside reverse transcriptase inhibitors (NRTI) treatment; HIV-associated neuropathy; schizophrenia; bipolar disorder; age-associated diseases; cerebral vascular diseases; macular degeneration; diabetes; and cancer.

### X. Pharmaceutical Compositions

Other aspects of the present disclosure relate to pharmaceutical compositions comprising any of the various components of the mtDNA editing system provided herein (e.g., deamination of mitochondrial DNA by a fusion protein or multiple fusion proteins) described herein (e.g., including, but not limited to, the mitoTALE, DddA, or portions thereof, and fusion proteins (e.g., comprising mitoTALE and portion of DddA)).

The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g. for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, the pharmaceutical composition is formulated for delivery to a subject (e.g., for nucleic acid editing). Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition is formulated in accordance with routine procedures as a composition adapted for intravenous or subcutaneous administration to a subject (e.g., a human). In some embodiments, pharmaceutical composition for administration by injection are solutions in sterile isotonic aqueous buffer. Where necessary, the pharmaceutical can also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid (e.g., sterile saline, lactated Ringer's or Hank's solution). In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising: (a) a container containing a compound of the invention in lyophilized form; and (b) a second container containing a pharmaceutically acceptable diluent (e.g., sterile water) for injection. The pharmaceutically acceptable diluent can be used for reconstitution or dilution of the lyophilized compound of the invention. Optionally associated with such container(s) can be a notice in the form prescribed by a governmental agency regulating the manufacture, use, or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### XI. Delivery Methods

In another aspect, the present disclosure provides for the delivery of Evolved DddA-containing base editors in vitro and in vivo using various strategies, including on separate vectors using split inteins and as well as direct delivery strategies of the ribonucleoprotein complex (i.e., the base editor complexed to the gRNA and/or the second-site gRNA) using techniques such as electroporation, use of cationic lipid-mediated formulations, and induced endocytosis methods using receptor ligands fused to the ribonucleoprotein complexes. In addition, mRNA delivery methods may also be employed. Any such methods are contemplated herein. The mtDNA BE fusion proteins, or components thereof, preferably be modified with an MTS or other signal sequence that facilitates entry of the polypeptides and the guide RNAs (in the case where a pDNAbp is Cas9) into the mitochondria.

In some aspects, the invention provides methods comprising delivering one or more base editor-encoding and/or gRNA-encoding polynucleotides, such as or one or more vectors as described herein encoding one or more components described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a base editor as described herein in combination with (and optionally complexed with) a guide sequence is delivered to a cell. Conventional viral and non-viral based gene transfer methods can be used to introduce nucleic acids in mammalian cells or target tissues. Such methods can be used to administer nucleic acids encoding components of a base editor to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bihm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration). The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a viruses can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

In various embodiments, the base editor constructs (including, the split-constructs) may be engineered for delivery in one or more rAAV vectors. An rAAV as related to any of the methods and compositions provided herein may be of any serotype including any derivative or pseudotype (e.g., 1, 2, 3, 4, 5, 6, 7, 8, 9, 10, 11, 12, 13, 2/1, 2/5, 2/8, 2/9, 3/1, 3/5, 3/8, or 3/9). An rAAV may comprise a genetic load (i.e., a recombinant nucleic acid vector that expresses a gene of interest, such as a whole or split base editor fusion protein that is carried by the rAAV into a cell) that is to be delivered to a cell. An rAAV may be chimeric.

As used herein, the serotype of an rAAV refers to the serotype of the capsid proteins of the recombinant virus. Non-limiting examples of derivatives and pseudotypes include rAAV2/1, rAAV2/5, rAAV2/8, rAAV2/9, AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y->F), AAV8 (Y733F), AAV2.15, AAV2.4, AAVM41, and AAVr3.45. A non-limiting example of derivatives and pseudotypes that have chimeric VP1 proteins is rAAV2/5-1VP1u, which has the genome of AAV2, capsid backbone of AAV5 and VP1u of AAV1. Other non-limiting example of derivatives and pseudotypes that have chimeric VP1 proteins are rAAV2/5-8VP1u, rAAV2/9-1VP1u, and rAAV2/9-8VP1u.

AAV derivatives/pseudotypes, and methods of producing such derivatives/pseudotypes are known in the art (see, e.g., Mol Ther. 2012 April; 20(4):699-708. doi: 10.1038/mt.2011.287. Epub 2012 Jan. 24. The AAV vector toolkit: poised at the clinical crossroads. Asokan A1, Schaffer D V, Samulski R J.). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Methods of making or packaging rAAV particles are known in the art and reagents are commercially available (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). For example, a plasmid comprising a gene of interest may be combined with one or more helper plasmids, e.g., that contain a rep gene (e.g., encoding Rep78, Rep68, Rep52 and Rep40) and a cap gene (encoding VP1, VP2, and VP3, including a modified VP2 region as described herein), and transfected into a recombinant cells such that the rAAV particle can be packaged and subsequently purified.

Recombinant AAV may comprise a nucleic acid vector, which may comprise at a minimum: (a) one or more heterologous nucleic acid regions comprising a sequence encoding a protein or polypeptide of interest or an RNA of interest (e.g., a siRNA or microRNA), and (b) one or more regions comprising inverted terminal repeat (ITR) sequences (e.g., wild-type ITR sequences or engineered ITR sequences) flanking the one or more nucleic acid regions (e.g., heterologous nucleic acid regions). Herein, heterologous nucleic acid regions comprising a sequence encoding a protein of interest or RNA of interest are referred to as genes of interest.

Any one of the rAAV particles provided herein may have capsid proteins that have amino acids of different serotypes outside of the VP1u region. In some embodiments, the serotype of the backbone of the VP1 protein is different from the serotype of the ITRs and/or the Rep gene. In some embodiments, the serotype of the backbone of the VP1 capsid protein of a particle is the same as the serotype of the ITRs. In some embodiments, the serotype of the backbone of the VP1 capsid protein of a particle is the same as the serotype of the Rep gene. In some embodiments, capsid proteins of rAAV particles comprise amino acid mutations that result in improved transduction efficiency.

In some embodiments, the nucleic acid vector comprises one or more regions comprising a sequence that facilitates expression of the nucleic acid (e.g., the heterologous nucleic acid), e.g., expression control sequences operatively linked to the nucleic acid. Numerous such sequences are known in the art. Non-limiting examples of expression control sequences include promoters, insulators, silencers, response elements, introns, enhancers, initiation sites, termination signals, and poly(A) tails. Any combination of such control sequences is contemplated herein (e.g., a promoter and an enhancer).

Final AAV constructs may incorporate a sequence encoding the gRNA. In other embodiments, the AAV constructs may incorporate a sequence encoding the second-site nicking guide RNA. In still other embodiments, the AAV constructs may incorporate a sequence encoding the second-site nicking guide RNA and a sequence encoding the gRNA.

In various embodiments, the gRNAs can be expressed from an appropriate promoter, such as a human U6 (hU6) promoter, a mouse U6 (mU6) promoter, or other appropriate promoter. The gRNAs (if multiple) can be driven by the same promoters or different promoters.

In some embodiments, a rAAV constructs or the herein compositions are administered to a subject enterally. In some embodiments, a rAAV constructs or the herein compositions are administered to the subject parenterally. In some embodiments, a rAAV particle or the herein compositions are administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs. In some embodiments, a rAAV particle or the herein compositions are administered to the subject by injection into the hepatic artery or portal vein.

In other aspects, the base editors can be divided at a split site and provided as two halves of a whole/complete base editor. The two halves can be delivered to cells (e.g., as expressed proteins or on separate expression vectors) and once in contact inside the cell, the two halves form the complete base editor through the self-splicing action of the inteins on each base editor half. Split intein sequences can be engineered into each of the halves of the encoded base editor to facilitate their transplicing inside the cell and the concomitant restoration of the complete, functioning base editor.

These split intein-based methods overcome several barriers to in vivo delivery. For example, the DNA encoding base editors is larger than the rAAV packaging limit, and so requires special solutions. One such solution is formulating the editor fused to split intein pairs that are packaged into two separate rAAV particles that, when co-delivered to a cell, reconstitute the functional editor protein.

In this aspect, the base editors can be divided at a split site and provided as two halves of a whole/complete base editor. The two halves can be delivered to cells (e.g., as expressed proteins or on separate expression vectors) and once in contact inside the cell, the two halves form the complete base editor through the self-splicing action of the inteins on each base editor half. Split intein sequences can be engineered into each of the halves of the encoded base editor to facilitate their transplicing inside the cell and the concomitant restoration of the complete, functioning base editor.

In various embodiments, the base editors may be engineered as two half proteins (i.e., a ABE N-terminal half and a CBE C-terminal half) by “splitting” the whole base editor as a “split site.” The “split site” refers to the location of insertion of split intein sequences (i.e., the N intein and the C intein) between two adjacent amino acid residues in the base editor. More specifically, the “split site” refers to the location of dividing the whole base editor into two separate halves, wherein in each halve is fused at the split site to either the N intein or the C intein motifs. The split site can be at any suitable location in the base editor fusion protein, but preferably the split site is located at a position that allows for the formation of two half proteins which are appropriately sized for delivery (e.g., by expression vector) and wherein the inteins, which are fused to each half protein at the split site termini, are available to sufficiently interact with one another when one half protein contacts the other half protein inside the cell.

In some embodiments, the split site is located in the pDNAbp domain. In other embodiments, the split site is located in the double stranded deaminase domain (DddA). In other embodiments, the split site is located in a linker that joins the napDNAbp domain and the deaminase domain. Preferably, the DddA is split so as to inactive the deaminase activity until the split fragments are co-localized in the mitochondria at the target site.

In various embodiments, split site design requires finding sites to split and insert an N- and C-terminal intein that are both structurally permissive for purposes of packaging the two half base editor domains into two different AAV genomes. Additionally, intein residues necessary for trans splicing can be incorporated by mutating residues at the N terminus of the C terminal extein or inserting residues that will leave an intein “scar.”

In various embodiments, using SpCas9 nickase as an example, the split can be between any two amino acids between 1 and 1368 of SEQ ID NO: 59. Preferred splits, however, will be located between the central region of the protein, e.g., from amino acids 50-1250, or from 100-1200, or from 150-1150, or from 200-1100, or from 250-1050, or from 300-1000, or from 350-950, or from 400-900, or from 450-850, or from 500-800, or from 550-750, or from 600-700 of SEQ ID NO: 59. In specific exemplary embodiments, the split site may be between 740/741, or 801/802, or 1010/1011, or 1041/1042. In other embodiments the split site may be between 1/2, 2/3, 3/4, 4/5, 5/6, 6/7, 7/8, 8/9, 9/10, 10/11, 12/13, 14/15, 15/16, 17/18, 19/20 . . . 50/51 . . . 100/101 . . . 200/201 . . . 300/301 . . . 400/401 . . . 500/501 . . . 600/601 . . . 700/701 . . . 800/801 . . . 900/901 . . . 1000/1001 . . . 1100/1101 . . . 1200/1201 . . . 1300/1301 . . . and 1367/1368, including all adjacent pairs of amino acid residues.

In various embodiments, the split inteins can be used to separately deliver separate portions of a complete Base editor fusion protein to a cell, which upon expression in a cell, become reconstituted as a complete Base editor fusion protein through the trans splicing.

In some embodiments, the disclosure provides a method of delivering a Base editor fusion protein to a cell, comprising: constructing a first expression vector encoding an N-terminal fragment of the Base editor fusion protein fused to a first split intein sequence; constructing a second expression vector encoding a C-terminal fragment of the Base editor fusion protein fused to a second split intein sequence; delivering the first and second expression vectors to a cell, wherein the N-terminal and C-terminal fragment are reconstituted as the Base editor fusion protein in the cell as a result of trans splicing activity causing self-excision of the first and second split intein sequences.

In other embodiments, the split site is in the napDNAbp domain.

In still other embodiments, the split site is in the deaminase domain.

In yet other embodiments, the split site is in the linker.

In other embodiments, the base editors may be delivered by ribonucleoprotein complexes.

In this aspect, the base editors may be delivered by non-viral delivery strategies involving delivery of a base editor complexed with a gRNA (i.e., a ABE ribonucleoprotein complex) by various methods, including electroporation and lipid nanoparticles. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

### XII. Kits, Vectors, Cells

Some aspects of this disclosure provide kits comprising a fusion protein or a nucleic acid construct comprising a nucleotide sequence encoding the various components (e.g., fusion protein) of the mtDNA editing system provided herein (e.g., deamination of mitochondrial DNA by a fusion protein or multiple fusion proteins) described herein (e.g., including, but not limited to, the mitoTALE-DddA fusion proteins, vectors or cells comprising the same). In some embodiments, the nucleotide sequence comprises a heterologous promoter that drives expression of the fusion protein editing system components described herein.

Some aspects of this disclosure provide kits comprising one or more fusion proteins or nucleic acid constructs encoding the various components of the mtDNA editing system provided herein (e.g., deamination of mitochondrial DNA by a fusion protein or multiple fusion proteins) described herein, e.g., the comprising a nucleotide sequence encoding the components of the mtDNA editing system provided herein (e.g., deamination of mitochondrial DNA by a fusion protein or multiple fusion proteins) capable of modifying a target DNA sequence. In some embodiments, the nucleotide sequence comprises a heterologous promoter that drives expression of the mtDNA editing system provided herein (e.g., deamination of mitochondrial DNA by a fusion protein or multiple fusion proteins) components.

In some embodiments, a kit further comprises a set of instructions for using the fusion proteins and/or carrying out the methods herein.

Some aspects of this disclosure provides kits comprising a nucleic acid construct, comprising (a) a nucleotide sequence encoding a fusion protein (e.g., a mitoTALE and portion of a DddA) and (b) a heterologous promoter that drives expression of the sequence of (a).

Some aspects of this disclosure provide cells comprising any of the constructs disclosed herein. In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a fusion protein system as described herein (such as by transient transfection of one or more vectors, or transfection with RNA), and modified through the activity of a fusion protein complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

### XIII. Sequences

In addition to the herein described evolved DddA proteins, the following sequences form a part of disclosure. Any of the following DddA proteins may be used as a starting point sequence to apply a continuous evolution process (e.g., described in FIG. 2) to obtain an evolved DddA variant for use in the base editors described herein. In addition, any of the following described fusion proteins having a DddA domain may be modified by evolving the DddA protein using one or more continuous evolutionary processes, such as PACE, described herein. The below sequences are contemplated, as well as fragments or variants thereof. including amino acid sequences having at least 70%, at least 75%, at least 80%, at least 85%, at least 90%, at least 95%, at least 96%, at least 97%, at least 98%, at least 99%, or 100% sequence identity with any of the below sequences.

### Split-DddAₜₒₓ-Cas9 Fusions Sequences

(A)G1397 DddAₜₒₓ₋N
(SEQ ID NO: 329)
GGCAGCTACGCCCTGGGTCCGTATCAGATTAGCGCCCCGCAGCTGCCAGCTTACAATGGTCAGAC
CGTGGGTACCTTCTACTATGTGAACGACGCGGGCGGTCTGGAGAGCAAGGTGTTTAGCAGCGGCG
GTCCAACCCCGTACCCAAACTATGCCAATGCCGGTCATGTGGAGGGTCAGAGCGCCCTGTTCATG
CGTGATAACGGCATCAGCGAGGGTCTGGTGTTCCACAACAACCCGGAAGGCACCTGCGGTTTTTG
CGTGAACATGACCGAGACCCTGCTGCCGGAAAACGCGAAAATGACCGTGGTGCCGCCGGAAGGT
Translated amino acid sequence:
(SEQ ID NO: 340)
GSYALGPYQISAPQLPAYNGQTVGTFYYVNDAGGLESKVFSSGGPTPYPNYANAGHVEGQSALFMRD
NGISEGLVFHNNPEGTCGFCVNMTETLLPENAKMTVVPPEG
(B) G1397 DddAₜₒₓ₋C
(SEQ ID NO: 330)
GCCATTCCAGTGAAGCGCGGCGCTACCGGTGAAACCAAAGTGTTTACCGGTAACAGCAACAGCC
CGAAGAGCCCGACCAAAGGCGGTTGC
Translated amino acid sequence:
(SEQ ID NO: 341)
AIPVKRGATGETKVFTGNSNSPKSPTKGGC

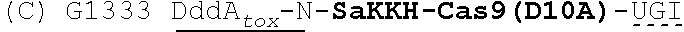

(SEQ ID NO: 331)
GGCAGCTACGCCCTGGGTCCGTATCAGATTAGCGCCCCGCAGCTGCCAGCTTACAATGGTCAGAC
CGTGGGTACCTTCTACTATGTGAACGACGCGGGCGGTCTGGAGAGCAAGGTGTTTAGCAGCGGCG
GTTCTGGTGGTTCTTCTGGTGGTTCTAGCGGCAGCGAGACTCCCGGGACCTCAGAGTCCGCCACA
CCCGAAAGTAGTGGCGGCAGCAGCGGCGGCAGCGGGAAACGGAACTACATCCTGGGGCTTGC
CATTGGGATAACCAGCGTTGGCTACGGAATTATTGATTATGAGACACGCGATGTGATTGAC
GCCGGGGTTAGGCTGTTCAAAGAGGCCAACGTTGAAAACAACGAGGGAAGACGGAGTAAG
CGCGGAGCAAGAAGACTCAAGCGCAGACGGAGACATCGGATTCAGAGGGTGAAAAAGCTG
CTCTTCGATTACAATCTCCTGACCGATCATAGTGAGCTGAGCGGAATCAACCCCTACGAGG
CGCGAGTGAAAGGGCTTTCCCAGAAGCTGTCCGAAGAGGAGTTCTCCGCCGCGTTGCTGCA
CCTGGCCAAACGGAGGGGGGTTCACAATGTAAACGAAGTGGAGGAGGACACGGGCAATGA
ACTTAGTACGAAAGAACAGATCAGTAGGAACTCTAAGGCTCTCGAAGAGAAATACGTCGCT
GAGTTGCAGCTTGAGAGACTGAAAAAAGACGGCGAAGTACGCGGATCTATTAATAGGTTCA
AGACTTCAGATTACGTAAAGGAAGCCAAGCAGCTCCTGAAAGTACAGAAAGCGTACCATCA
GCTCGATCAGAGCTTCATCGATACCTACATAGATTTGCTGGAGACACGGAGGACATACTAC
GAGGGCCCAGGGGAAGGATCTCCTTTTGGGTGGAAGGACATCAAGGAATGGTACGAGATG
CTTATGGGACATTGTACATATTTTCCGGAGGAGCTCAGGAGCGTCAAGTACGCCTACAATG
CCGACCTGTACAATGCCCTCAATGACCTCAATAACCTCGTGATTACCAGGGACGAGAACGA
GAAGCTGGAGTACTATGAAAAGTTCCAGATTATCGAGAATGTGTTTAAGCAGAAGAAGAAG
CCGACACTTAAGCAGATTGCAAAGGAAATCCTCGTGAATGAGGAAGATATCAAGGGATACA
GAGTGACAAGTACAGGCAAGCCCGAGTTCACAAATCTGAAGGTGTACCACGATATTAAGGA
CATAACCGCACGAAAGGAGATAATCGAAAACGCTGAGCTCCTCGATCAGATCGCAAAAATT
CTTACCATCTACCAGTCTAGTGAGGACATTCAGGAGGAACTGACTAATCTGAACAGTGAGC
TCACCCAAGAGGAAATTGAGCAGATTTCAAACCTGAAAGGCTACACCGGGACGCACAATCT
GAGCCTCAAAGCAATCAACCTCATTCTGGATGAACTTTGGCACACAAATGACAACCAAATTG
CCATATTCAACCGCCTGAAACTGGTGCCAAAAAAAGTGGATCTGTCACAGCAAAAGGAAAT
CCCTACAACCTTGGTTGACGATTTTATTCTGTCCCCCGTTGTCAAGCGGAGCTTCATCCAGT
CAATCAAGGTGATCAATGCCATCATTAAAAAATACGGATTGCCAAACGATATAATTATCGAG
CTTGCACGAGAGAAGAACTCAAAGGACGCCCAGAAGATGATTAACGAAATGCAGAAGCGCA
ACCGCCAGACAAACGAACGCATAGAGGAAATTATAAGAACAACCGGCAAAGAGAATGCCAA
GTATCTGATCGAGAAAATCAAGCTGCACGACATGCAAGAAGGCAAGTGCCTGTACTCTCTG
GAAGCTATCCCACTCGAAGATCTGCTGAATAATCCATTCAATTACGAGGTGGACCACATCAT
CCCTAGATCCGTAAGCTTTGACAATTCCTTCAATAACAAAGTTCTGGTTAAACAGGAGGAAA
ATTCTAAAAAAGGGAACCGGACCCCGTTCCAGTACCTGAGCTCCAGTGACAGCAAGATTAG
CTACGAGACTTTTAAGAAACATATTCTGAATCTGGCCAAAGGCAAAGGCAGGATCAGCAAG
ACCAAGAAGGAGTACCTCCTCGAAGAACGCGACATTAACAGATTTAGTGTGCAGAAAGATT
TCATCAACCGAAACCTTGTCGATACTCGGTACGCCACGAGAGGCCTGATGAATCTCCTCAG
GAGCTACTTCCGCGTCAATAATCTGGACGTTAAAGTCAAGAGCATAAATGGGGGATTCACC
AGCTTTCTGAGGAGAAAGTGGAAGTTTAAGAAGGAACGAAACAAAGGATACAAGCACCATG
CTGAGGATGCTTTGATCATCGCTAACGCGGACTTTATCTTTAAGGAATGGAAAAAGCTGGAT
AAGGCAAAGAAAGTGATGGAAAACCAGATGTTCGAGGAGAAGCAGGCAGAGTCAATGCCT
GAGATCGAGACAGAGCAGGAATACAAGGAAATTTTCATCACCCCTCATCAGATTAAACACA
TAAAGGACTTCAAAGACTATAAATACTCTCATAGGGTGGACAAAAAACCCAATCGCAAGCTC
ATTAATGACACCCTGTACTCAACACGGAAGGATGATAAAGGTAATACCTTGATTGTGAATAA
TCTTAATGGATTGTATGACAAAGATAACGACAAGCTCAAGAAGCTGATCAACAAGTCTCCAG
AGAAGCTCCTTATGTATCACCACGACCCACAGACTTATCAGAAATTGAAACTGATCATGGAG
CAATACGGGGATGAGAAGAACCCACTCTACAAATATTATGAGGAAACAGGTAATTACCTGA
CCAAGTACTCCAAGAAGGATAACGGACCAGTGATCAAAAAGATAAAGTACTATGGCAACAA
ACTTAATGCGCATTTGGACATAACTGACGATTACCCCAATTCTCGAAACAAGGTTGTGAAGC
TCTCCCTGAAGCCTTATAGATTTGACGTGTACCTGGATAATGGGGTTTATAAATTCGTCACC
GTGAAAAATCTGGACGTGATCAAAAAGGAGAACTATTATGAAGTAAACTCAAAGTGCTATG
AGGAGGCGAAGAAGCTGAAGAAGATCTCCAATCAGGCCGAGTTCATCGCTTCCTTCTATAA
GAACGATCTCATCAAGATCAATGGAGAGCTTTATCGCGTCATTGGTGTGAACAATGACTTGC
TGAACAGGATCGAAGTCAATATGATAGACATTACCTACCGGGAGTATCTCGAAAACATGAA
TGATAAACGGCCGCCTCACATCATCAAGACAATCGCATCTAAAACTCAGTCAATAAAAAAGT
ACTCTACCGATATCCTGGGGAATCTCTATGAAGTGAAGTCAAAGAAGCACCCACAAATCATT
AAAAAAGGTGGATCCCCCAAGAAGAAGAGGAAAGTCTCGAGCGACTACAAAGACCATGACG
GTGATTATAAAGATCATGACATCGATTACAAGGATGACGATGACAAGTCTGGTGGTTCTACTA

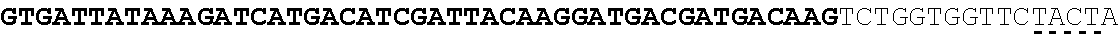

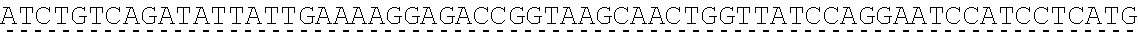

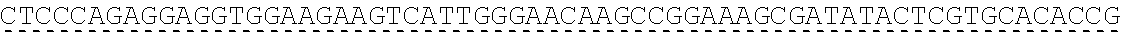

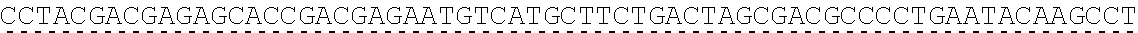

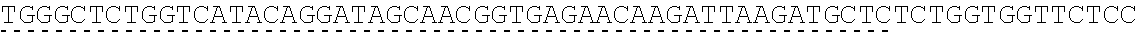

CAAGAAGAAGAGGAAAGTC
Translated amino acid sequence:
(SEQ ID NO: 332)
GSYALGPYQISAPQLPAYNGQTVGTFYYVNDAGGLESKVFSSGGSGGSSGGSSGSETPGTSESATPESS
GGSSGGSGKRNYILGLAIGITSVGYGIIDYETRDVIDAGVRLFKEANVENNEGRRSKRGARRLKRRRR
HRIQRVKKLLFDYNLLTDHSELSGINPYEARVKGLSQKLSEEEFSAALLHLAKRRGVHNVNEVEEDTG
NELSTKEQISRNSKALEEKYVAELQLERLKKDGEVRGSINRFKTSDYVKEAKQLLKVQKAYHQLDQS
FIDTYIDLLETRRTYYEGPGEGSPFGWKDIKEWYEMLMGHCTYFPEELRSVKYAYNADLYNALNDLN
NLVITRDENEKLEYYEKFQIIENVFKQKKKPTLKQIAKEILVNEEDIKGYRVTSTGKPEFTNLKVYHDIK
DITARKEIIENAELLDQIAKILTIYQSSEDIQEELTNLNSELTQEEIEQISNLKGYTGTHNLSLKAINLILDE
LWHTNDNQIAIFNRLKLVPKKVDLSQQKEIPTTLVDDFILSPVVKRSFIQSIKVINAIIKKYGLPNDIIIEL
AREKNSKDAQKMINEMQKRNRQTNERIEEIIRTTGKENAKYLIEKIKLHDMQEGKCLYSLEAIPLEDLL
NNPFNYEVDHIIPRSVSFDNSFNNKVLVKQEENSKKGNRTPFQYLSSSDSKISYETFKKHILNLAKGKG
RISKTKKEYLLEERDINRFSVQKDFINRNLVDTRYATRGLMNLLRSYFRVNNLDVKVKSINGGFTSFLR
RKWKFKKERNKGYKHHAEDALIIANADFIFKEWKKLDKAKKVMENQMFEEKQAESMPEIETEQEYK
EIFITPHQIKHIKDFKDYKYSHRVDKKPNRKLINDTLYSTRKDDKGNTLIVNNLNGLYDKDNDKLKKLI
NKSPEKLLMYHHDPQTYQKLKLIMEQYGDEKNPLYKYYEETGNYLTKYSKKDNGPVIKKIKYYGNK
LNAHLDITDDYPNSRNKVVKLSLKPYRFDVYLDNGVYKFVTVKNLDVIKKENYYEVNSKCYEEAKK
LKKISNQAEFIASFYKNDLIKINGELYRVIGVNNDLLNRIEVNMIDITYREYLENMNDKRPPHIIKTIASK
TQSIKKYSTDILGNLYEVKSKKHPQIIKKGGSPKKKRKVSSDYKDHDGDYKDHDIDYKDDDDKSGGS
TNLSDIIEKETGKQLVIQESILMLPEEVEEVIGNKPESDILVHTAYDESTDENVMLLTSDAPEYKPWALV
IQDSNGENKIKMLSGGSPKKKRKV

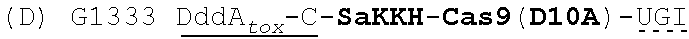

(SEQ ID NO: 333)
CCAACCCCGTACCCAAACTATGCCAATGCCGGTCATGTGGAGGGTCAGAGCGCCCTGTTCATGCG
TGATAACGGCATCAGCGAGGGTCTGGTGTTCCACAACAACCCGGAAGGCACCTGCGGTTTTTGCG
TGAACATGACCGAGACCCTGCTGCCGGAAAACGCGAAAATGACCGTGGTGCCGCCGGAAGGTGC
CATTCCAGTGAAGCGCGGCGCTACCGGTGAAACCAAAGTGTTTACCGGTAACAGCAACAGCCCG
AAGAGCCCGACCAAAGGCGGTTGCTCTGGTGGTTCTTCTGGTGGTTCTAGCGGCAGCGAGACTCC
CGGGACCTCAGAGTCCGCCACACCCGAAAGTAGTGGCGGCAGCAGCGGCGGCAGCGGGAAACG
GAACTACATCCTGGGGCTTGCCATTGGGATAACCAGCGTTGGCTACGGAATTATTGATTAT
GAGACACGCGATGTGATTGACGCCGGGGTTAGGCTGTTCAAAGAGGCCAACGTTGAAAACA
ACGAGGGAAGACGGAGTAAGCGCGGAGCAAGAAGACTCAAGCGCAGACGGAGACATCGGA
TTCAGAGGGTGAAAAAGCTGCTCTTCGATTACAATCTCCTGACCGATCATAGTGAGCTGAG
CGGAATCAACCCCTACGAGGCGCGAGTGAAAGGGCTTTCCCAGAAGCTGTCCGAAGAGGA
GTTCTCCGCCGCGTTGCTGCACCTGGCCAAACGGAGGGGGGTTCACAATGTAAACGAAGTG
GAGGAGGACACGGGCAATGAACTTAGTACGAAAGAACAGATCAGTAGGAACTCTAAGGCTC
TCGAAGAGAAATACGTCGCTGAGTTGCAGCTTGAGAGACTGAAAAAAGACGGCGAAGTACG
CGGATCTATTAATAGGTTCAAGACTTCAGATTACGTAAAGGAAGCCAAGCAGCTCCTGAAA
GTACAGAAAGCGTACCATCAGCTCGATCAGAGCTTCATCGATACCTACATAGATTTGCTGGA
GACACGGAGGACATACTACGAGGGCCCAGGGGAAGGATCTCCTTTTGGGTGGAAGGACAT
CAAGGAATGGTACGAGATGCTTATGGGACATTGTACATATTTTCCGGAGGAGCTCAGGAGC
GTCAAGTACGCCTACAATGCCGACCTGTACAATGCCCTCAATGACCTCAATAACCTCGTGAT
TACCAGGGACGAGAACGAGAAGCTGGAGTACTATGAAAAGTTCCAGATTATCGAGAATGTG
TTTAAGCAGAAGAAGAAGCCGACACTTAAGCAGATTGCAAAGGAAATCCTCGTGAATGAGG
AAGATATCAAGGGATACAGAGTGACAAGTACAGGCAAGCCCGAGTTCACAAATCTGAAGGT
GTACCACGATATTAAGGACATAACCGCACGAAAGGAGATAATCGAAAACGCTGAGCTCCTC
GATCAGATCGCAAAAATTCTTACCATCTACCAGTCTAGTGAGGACATTCAGGAGGAACTGA
CTAATCTGAACAGTGAGCTCACCCAAGAGGAAATTGAGCAGATTTCAAACCTGAAAGGCTA
CACCGGGACGCACAATCTGAGCCTCAAAGCAATCAACCTCATTCTGGATGAACTTTGGCAC
ACAAATGACAACCAAATTGCCATATTCAACCGCCTGAAACTGGTGCCAAAAAAAGTGGATCT
GTCACAGCAAAAGGAAATCCCTACAACCTTGGTTGACGATTTTATTCTGTCCCCCGTTGTCA
AGCGGAGCTTCATCCAGTCAATCAAGGTGATCAATGCCATCATTAAAAAATACGGATTGCCA
AACGATATAATTATCGAGCTTGCACGAGAGAAGAACTCAAAGGACGCCCAGAAGATGATTA
ACGAAATGCAGAAGCGCAACCGCCAGACAAACGAACGCATAGAGGAAATTATAAGAACAAC
CGGCAAAGAGAATGCCAAGTATCTGATCGAGAAAATCAAGCTGCACGACATGCAAGAAGGC
AAGTGCCTGTACTCTCTGGAAGCTATCCCACTCGAAGATCTGCTGAATAATCCATTCAATTA
CGAGGTGGACCACATCATCCCTAGATCCGTAAGCTTTGACAATTCCTTCAATAACAAAGTTC
TGGTTAAACAGGAGGAAAATTCTAAAAAAGGGAACCGGACCCCGTTCCAGTACCTGAGCTC
CAGTGACAGCAAGATTAGCTACGAGACTTTTAAGAAACATATTCTGAATCTGGCCAAAGGC
AAAGGCAGGATCAGCAAGACCAAGAAGGAGTACCTCCTCGAAGAACGCGACATTAACAGAT
TTAGTGTGCAGAAAGATTTCATCAACCGAAACCTTGTCGATACTCGGTACGCCACGAGAGG
CCTGATGAATCTCCTCAGGAGCTACTTCCGCGTCAATAATCTGGACGTTAAAGTCAAGAGCA
TAAATGGGGGATTCACCAGCTTTCTGAGGAGAAAGTGGAAGTTTAAGAAGGAACGAAACAA
AGGATACAAGCACCATGCTGAGGATGCTTTGATCATCGCTAACGCGGACTTTATCTTTAAGG
AATGGAAAAAGCTGGATAAGGCAAAGAAAGTGATGGAAAACCAGATGTTCGAGGAGAAGCA
GGCAGAGTCAATGCCTGAGATCGAGACAGAGCAGGAATACAAGGAAATTTTCATCACCCCT
CATCAGATTAAACACATAAAGGACTTCAAAGACTATAAATACTCTCATAGGGTGGACAAAAA
ACCCAATCGCAAGCTCATTAATGACACCCTGTACTCAACACGGAAGGATGATAAAGGTAAT
ACCTTGATTGTGAATAATCTTAATGGATTGTATGACAAAGATAACGACAAGCTCAAGAAGCT
GATCAACAAGTCTCCAGAGAAGCTCCTTATGTATCACCACGACCCACAGACTTATCAGAAAT
TGAAACTGATCATGGAGCAATACGGGGATGAGAAGAACCCACTCTACAAATATTATGAGGA
AACAGGTAATTACCTGACCAAGTACTCCAAGAAGGATAACGGACCAGTGATCAAAAAGATA
AAGTACTATGGCAACAAACTTAATGCGCATTTGGACATAACTGACGATTACCCCAATTCTCG
AAACAAGGTTGTGAAGCTCTCCCTGAAGCCTTATAGATTTGACGTGTACCTGGATAATGGG
GTTTATAAATTCGTCACCGTGAAAAATCTGGACGTGATCAAAAAGGAGAACTATTATGAAGT
AAACTCAAAGTGCTATGAGGAGGCGAAGAAGCTGAAGAAGATCTCCAATCAGGCCGAGTTC
ATCGCTTCCTTCTATAAGAACGATCTCATCAAGATCAATGGAGAGCTTTATCGCGTCATTGG
TGTGAACAATGACTTGCTGAACAGGATCGAAGTCAATATGATAGACATTACCTACCGGGAG
TATCTCGAAAACATGAATGATAAACGGCCGCCTCACATCATCAAGACAATCGCATCTAAAAC
TCAGTCAATAAAAAAGTACTCTACCGATATCCTGGGGAATCTCTATGAAGTGAAGTCAAAGA
AGCACCCACAAATCATTAAAAAAGGTGGATCCCCCAAGAAGAAGAGGAAAGTCTCGAGCGA
CTACAAAGACCATGACGGTGATTATAAAGATCATGACATCGATTACAAGGATGACGATGAC

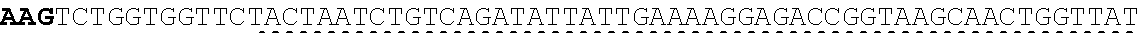

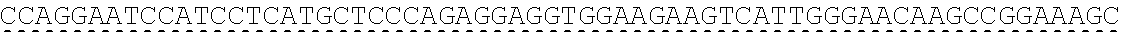

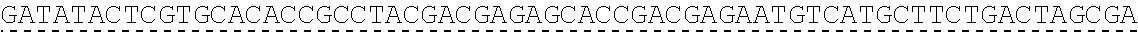

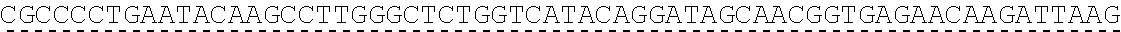

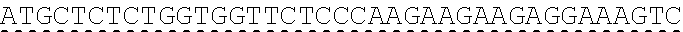

Translated amino acid sequence:
(SEQ ID NO: 334)
PTPYPNYANAGHVEGQSALFMRDNGISEGLVFHNNPEGTCGFCVNMTETLLPENAKMTVVPPEGAIP
VKRGATGETKVFTGNSNSPKSPTKGGCSGGSSGGSSGSETPGTSESATPESSGGSSGGSGKRNYILGLAI
GITSVGYGIIDYETRDVIDAGVRLFKEANVENNEGRRSKRGARRLKRRRRHRIQRVKKLLFDYNLLTD
HSELSGINPYEARVKGLSQKLSEEEFSAALLHLAKRRGVHNVNEVEEDTGNELSTKEQISRNSKALEE
KYVAELQLERLKKDGEVRGSINRFKTSDYVKEAKQLLKVQKAYHQLDQSFIDTYIDLLETRRTYYEGP
GEGSPFGWKDIKEWYEMLMGHCTYFPEELRSVKYAYNADLYNALNDLNNLVITRDENEKLEYYEKF
QIIENVFKQKKKPTLKQIAKEILVNEEDIKGYRVTSTGKPEFTNLKVYHDIKDITARKEIIENAELLDQIA
KILTIYQSSEDIQEELTNLNSELTQEEIEQISNLKGYTGTHNLSLKAINLILDELWHTNDNQIAIFNRLKL
VPKKVDLSQQKEIPTTLVDDFILSPVVKRSFIQSIKVINAIIKKYGLPNDIIIELAREKNSKDAQKMINEM
QKRNRQTNERIEEIIRTTGKENAKYLIEKIKLHDMQEGKCLYSLEAIPLEDLLNNPFNYEVDHIIPRSVSF
DNSFNNKVLVKQEENSKKGNRTPFQYLSSSDSKISYETFKKHILNLAKGKGRISKTKKEYLLEERDINR
FSVQKDFINRNLVDTRYATRGLMNLLRSYFRVNNLDVKVKSINGGFTSFLRRKWKFKKERNKGYKH
HAEDALIIANADFIFKEWKKLDKAKKVMENQMFEEKQAESMPEIETEQEYKEIFITPHQIKHIKDFKDY
KYSHRVDKKPNRKLINDTLYSTRKDDKGNTLIVNNLNGLYDKDNDKLKKLINKSPEKLLMYHHDPQT
YQKLKLIMEQYGDEKNPLYKYYEETGNYLTKYSKKDNGPVIKKIKYYGNKLNAHLDITDDYPNSRNK
VVKLSLKPYRFDVYLDNGVYKFVTVKNLDVIKKENYYEVNSKCYEEAKKLKKISNQAEFIASFYKND
LIKINGELYR VIGVNNDLLNRIEVNMIDITYREYLENMNDKRPPHIIKTIASKTQSIKKYSTDILGNLYEV
KSKKHPQIIKKGGSPKKKRKVSSDYKDHDGDYKDHDIDYKDDDDKSGGSTNLSDIIEKETGKQLVIQE
SILMLPEEVEEVIGNKPESDILVHTAYDESTDENVMLLTSDAPEYKPWALVIQDSNGENKIKMLSGGSP
KKKRKV
(E) G1333 DddAₜₒₓ₋N-dSpCas9-2x-UGI
(SEQ ID NO: 335)
AAACGGACAGCCGACGGAAGCGAGTTCGAGTCACCAAAGAAGAAGCGGAAAGTCGGCAGCTAC
GCCCTGGGTCCGTATCAGATTAGCGCCCCGCAGCTGCCAGCTTACAATGGTCAGACCGTGGGTAC
CTTCTACTATGTGAACGACGCGGGCGGTCTGGAGAGCAAGGTGTTTAGCAGCGGCGGTTCTGGAG
GATCTAGCGGAGGATCCTCTGGCAGCGAGACACCAGGAACAAGCGAGTCAGCAACACCAGAGAG
CAGTGGCGGCAGCAGCGGCGGCAGCGACAAGAAGTACAGCATCGGCCTGGCCATCGGCACCA
ACTCTGTGGGCTGGGCCGTGATCACCGACGAGTACAAGGTGCCCAGCAAGAAATTCAAGGT
GCTGGGCAACACCGACCGGCACAGCATCAAGAAGAACCTGATCGGAGCCCTGCTGTTCGAC
AGCGGCGAAACAGCCGAGGCCACCCGGCTGAAGAGAACCGCCAGAAGAAGATACACCAGA
CGGAAGAACCGGATCTGCTATCTGCAAGAGATCTTCAGCAACGAGATGGCCAAGGTGGACG
ACAGCTTCTTCCACAGACTGGAAGAGTCCTTCCTGGTGGAAGAGGATAAGAAGCACGAGCG
GCACCCCATCTTCGGCAACATCGTGGACGAGGTGGCCTACCACGAGAAGTACCCCACCATC
TACCACCTGAGAAAGAAACTGGTGGACAGCACCGACAAGGCCGACCTGCGGCTGATCTATC
TGGCCCTGGCCCACATGATCAAGTTCCGGGGCCACTTCCTGATCGAGGGCGACCTGAACCC
CGACAACAGCGACGTGGACAAGCTGTTCATCCAGCTGGTGCAGACCTACAACCAGCTGTTC
GAGGAAAACCCCATCAACGCCAGCGGCGTGGACGCCAAGGCCATCCTGTCTGCCAGACTGA
GCAAGAGCAGACGGCTGGAAAATCTGATCGCCCAGCTGCCCGGCGAGAAGAAGAATGGCC
TGTTCGGAAACCTGATTGCCCTGAGCCTGGGCCTGACCCCCAACTTCAAGAGCAACTTCGA
CCTGGCCGAGGATGCCAAACTGCAGCTGAGCAAGGACACCTACGACGACGACCTGGACAAC
CTGCTGGCCCAGATCGGCGACCAGTACGCCGACCTGTTTCTGGCCGCCAAGAACCTGTCCG
ACGCCATCCTGCTGAGCGACATCCTGAGAGTGAACACCGAGATCACCAAGGCCCCCCTGAG
CGCCTCTATGATCAAGAGATACGACGAGCACCACCAGGACCTGACCCTGCTGAAAGCTCTC
GTGCGGCAGCAGCTGCCTGAGAAGTACAAAGAGATTTTCTTCGACCAGAGCAAGAACGGCT
ACGCCGGCTACATTGACGGCGGAGCCAGCCAGGAAGAGTTCTACAAGTTCATCAAGCCCAT
CCTGGAAAAGATGGACGGCACCGAGGAACTGCTCGTGAAGCTGAACAGAGAGGACCTGCT
GCGGAAGCAGCGGACCTTCGACAACGGCAGCATCCCCCACCAGATCCACCTGGGAGAGCT
GCACGCCATTCTGCGGCGGCAGGAAGATTTTTACCCATTCCTGAAGGACAACCGGGAAAAG
ATCGAGAAGATCCTGACCTTCCGCATCCCCTACTACGTGGGCCCTCTGGCCAGGGGAAACA
GCAGATTCGCCTGGATGACCAGAAAGAGCGAGGAAACCATCACCCCCTGGAACTTCGAGGA
AGTGGTGGACAAGGGCGCTTCCGCCCAGAGCTTCATCGAGCGGATGACCAACTTCGATAAG
AACCTGCCCAACGAGAAGGTGCTGCCCAAGCACAGCCTGCTGTACGAGTACTTCACCGTGT
ATAACGAGCTGACCAAAGTGAAATACGTGACCGAGGGAATGAGAAAGCCCGCCTTCCTGAG
CGGCGAGCAGAAAAAGGCCATCGTGGACCTGCTGTTCAAGACCAACCGGAAAGTGACCGTG
AAGCAGCTGAAAGAGGACTACTTCAAGAAAATCGAGTGCTTCGACTCCGTGGAAATCTCCG
GCGTGGAAGATCGGTTCAACGCCTCCCTGGGCACATACCACGATCTGCTGAAAATTATCAA
GGACAAGGACTTCCTGGACAATGAGGAAAACGAGGACATTCTGGAAGATATCGTGCTGACC
CTGACACTGTTTGAGGACAGAGAGATGATCGAGGAACGGCTGAAAACCTATGCCCACCTGT
TCGACGACAAAGTGATGAAGCAGCTGAAGCGGCGGAGATACACCGGCTGGGGCAGGCTGA
GCCGGAAGCTGATCAACGGCATCCGGGACAAGCAGTCCGGCAAGACAATCCTGGATTTCCT
GAAGTCCGACGGCTTCGCCAACAGAAACTTCATGCAGCTGATCCACGACGACAGCCTGACC
TTTAAAGAGGACATCCAGAAAGCCCAGGTGTCCGGCCAGGGCGATAGCCTGCACGAGCACA
TTGCCAATCTGGCCGGCAGCCCCGCCATTAAGAAGGGCATCCTGCAGACAGTGAAGGTGGT
GGACGAGCTCGTGAAAGTGATGGGCCGGCACAAGCCCGAGAACATCGTGATCGAAATGGC
CAGAGAGAACCAGACCACCCAGAAGGGACAGAAGAACAGCCGCGAGAGAATGAAGCGGAT
CGAAGAGGGCATCAAAGAGCTGGGCAGCCAGATCCTGAAAGAACACCCCGTGGAAAACAC
CCAGCTGCAGAACGAGAAGCTGTACCTGTACTACCTGCAGAATGGGCGGGATATGTACGTG
GACCAGGAACTGGACATCAACCGGCTGTCCGACTACGATGTGGACGCTATCGTGCCTCAGA
GCTTTCTGAAGGACGACTCCATCGACAACAAGGTGCTGACCAGAAGCGACAAGAACCGGGG
CAAGAGCGACAACGTGCCCTCCGAAGAGGTCGTGAAGAAGATGAAGAACTACTGGCGGCA
GCTGCTGAACGCCAAGCTGATTACCCAGAGAAAGTTCGACAATCTGACCAAGGCCGAGAGA
GGCGGCCTGAGCGAACTGGATAAGGCCGGCTTCATCAAGAGACAGCTGGTGGAAACCCGG
CAGATCACAAAGCACGTGGCACAGATCCTGGACTCCCGGATGAACACTAAGTACGACGAGA
ATGACAAGCTGATCCGGGAAGTGAAAGTGATCACCCTGAAGTCCAAGCTGGTGTCCGATTT
CCGGAAGGATTTCCAGTTTTACAAAGTGCGCGAGATCAACAACTACCACCACGCCCACGAC
GCCTACCTGAACGCCGTCGTGGGAACCGCCCTGATCAAAAAGTACCCTAAGCTGGAAAGCG
AGTTCGTGTACGGCGACTACAAGGTGTACGACGTGCGGAAGATGATCGCCAAGAGCGAGCA
GGAAATCGGCAAGGCTACCGCCAAGTACTTCTTCTACAGCAACATCATGAACTTTTTCAAGA
CCGAGATTACCCTGGCCAACGGCGAGATCCGGAAGCGGCCTCTGATCGAGACAAACGGCG
AAACCGGGGAGATCGTGTGGGATAAGGGCCGGGATTTTGCCACCGTGCGGAAAGTGCTGA
GCATGCCCCAAGTGAATATCGTGAAAAAGACCGAGGTGCAGACAGGCGGCTTCAGCAAAGA
GTCTATCCTGCCCAAGAGGAACAGCGATAAGCTGATCGCCAGAAAGAAGGACTGGGACCCT
AAGAAGTACGGCGGCTTCGACAGCCCCACCGTGGCCTATTCTGTGCTGGTGGTGGCCAAAG
TGGAAAAGGGCAAGTCCAAGAAACTGAAGAGTGTGAAAGAGCTGCTGGGGATCACCATCAT
GGAAAGAAGCAGCTTCGAGAAGAATCCCATCGACTTTCTGGAAGCCAAGGGCTACAAAGAA
GTGAAAAAGGACCTGATCATCAAGCTGCCTAAGTACTCCCTGTTCGAGCTGGAAAACGGCC
GGAAGAGAATGCTGGCCTCTGCCGGCGAACTGCAGAAGGGAAACGAACTGGCCCTGCCCT
CCAAATATGTGAACTTCCTGTACCTGGCCAGCCACTATGAGAAGCTGAAGGGCTCCCCCGA
GGATAATGAGCAGAAACAGCTGTTTGTGGAACAGCACAAGCACTACCTGGACGAGATCATC
GAGCAGATCAGCGAGTTCTCCAAGAGAGTGATCCTGGCCGACGCTAATCTGGACAAAGTGC
TGTCCGCCTACAACAAGCACCGGGATAAGCCCATCAGAGAGCAGGCCGAGAATATCATCCA
CCTGTTTACCCTGACCAATCTGGGAGCCCCTGCCGCCTTCAAGTACTTTGACACCACCATCG
ACCGGAAGAGGTACACCAGCACCAAAGAGGTGCTGGACGCCACCCTGATCCACCAGAGCAT
CACCGGCCTGTACGAGACACGGATCGACCTGTCTCAGCTGGGAGGTGACAGCGGCGGGAGC

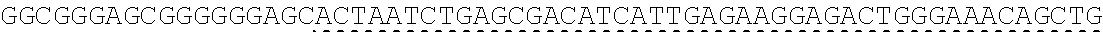

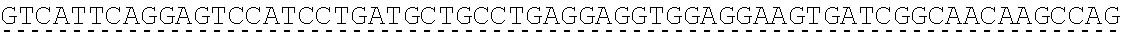

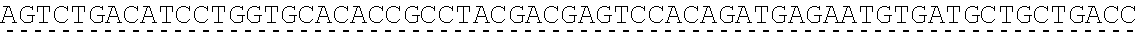

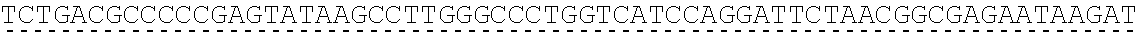

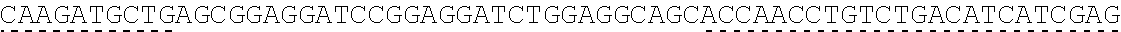

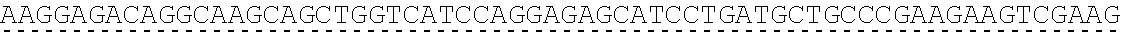

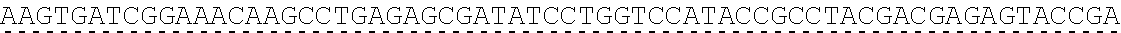

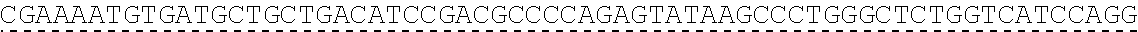

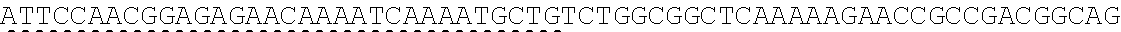

CGAATTCGAGCCCAAGAAGAAGAGGAAAGTC
Translated amino acid sequence:
(SEQ ID NO: 336)
KRTADGSEFESPKKKRKVGSYALGPYQISAPQLPAYNGQTVGTFYYVNDAGGLESKVFSSGGSGGSS
GGSSGSETPGTSESATPESSGGSSGGSDKKYSIGLAIGTNSVGWAVITDEYKVPSKKFKVLGNTDRHSI
KKNLIGALLFDSGETAEATRLKRTARRRYTRRKNRICYLQEIFSNEMAKVDDSFFHRLEESFLVEEDKK
HERHPIFGNIVDEVAYHEKYPTIYHLRKKL VDSTDKADLRLIYLALAHMIKFRGHFLIEGDLNPDNSDV
DKLFIQLVQTYNQLFEENPINASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKFIKPILEKMDGTEE
LLVKLNREDLLRKQRTFDNGSIPHQIHLGELHAILRRQEDFYPFLKDNREKIEKILTFRIPYYVGPLARG
NSRFAWMTRKSEETITPWNFEEVVDKGASAQSFIERMTNFDKNLPNEKVLPKHSLLYEYFTVYNELTK
VKYVTEGMRKPAFLSGEQKKAIVDLLFKTNRKVTVKQLKEDYFKKIECFDSVEISGVEDRFNASLGTY
HDLLKIIKDKDFLDNEENEDILEDIVLTLTLFEDREMIEERLKTYAHLFDDKVMKQLKRRRYTGWGRL
SRKLINGIRDKQSGKTILDFLKSDGFANRNFMQLIHDDSLTFKEDIQKAQVSGQGDSLHEHIANLAGSP
AIKKGILQTVKVVDELVKVMGRHKPENIVIEMARENQTTQKGQKNSRERMKRIEEGIKELGSQILKEH
PVENTQLQNEKLYLYYLQNGRDMYVDQELDINRLSDYDVDAIVPQSFLKDDSIDNKVLTRSDKNRGK
SDNVPSEEVVKKMKNYWRQLLNAKLITQRKFDNLTKAERGGLSELDKAGFIKRQLVETRQITKHVAQ
ILDSRMNTKYDENDKLIREVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEITLANGEIRKRPLIETNGET
GEIVWDKGRDFATVRKVLSMPQVNIVKKTEVQTGGFSKESILPKRNSDKLIARKKDWDPKKYGGFDS
PTVAYSVLVVAKVEKGKSKKLKSVKELLGITIMERSSFEKNPIDFLEAKGYKEVKKDLIIKLPKYSLFE
LENGRKRMLASAGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTIDRKRYTSTKEV
LDATLIHQSITGLYETRIDLSQLGGDSGGSGGSGGSTNLSDIIEKETGKQL VIQESILMLPEEVEEVIGNK
PESDIL VHTAYDESTDENVMLLTSDAPEYKPWALVIQDSNGENKIKMLSGGSGGSGGSTNLSDIIEKET
GKQLVIQESILMLPEEVEEVIGNKPESDILVHTAYDESTDENVMLLTSDAPEYKPWALVIQDSNGENKI
KMLSGGSKRTADGSEFEPKKKRKV

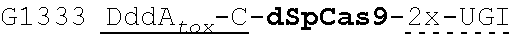

(SEQ ID NO: 337)
AAACGGACAGCCGACGGAAGCGAGTTCGAGTCACCAAAGAAGAAGCGGAAAGTCCCAACCCCGT
ACCCAAACTATGCCAATGCCGGTCATGTGGAGGGTCAGAGCGCCCTGTTCATGCGTGATAACGGC
ATCAGCGAGGGTCTGGTGTTCCACAACAACCCGGAAGGCACCTGCGGTTTTTGCGTGAACATGAC
CGAGACCCTGCTGCCGGAAAACGCGAAAATGACCGTGGTGCCGCCGGAAGGTGCCATTCCAGTG
AAGCGCGGCGCTACCGGTGAAACCAAAGTGTTTACCGGTAACAGCAACAGCCCGAAGAGCCCGA
CCAAAGGCGGTTGCTTCTGGAGGATCTAGCGGAGGATCCTCTGGCAGCGAGACACCAGGAACAA
GCGAGTCAGCAACACCAGAGAGCAGTGGCGGCAGCAGCGGCGGCAGCGACAAGAAGTACAGCA
TCGGCCTGGCCATCGGCACCAACTCTGTGGGCTGGGCCGTGATCACCGACGAGTACAAGGT
GCCCAGCAAGAAATTCAAGGTGCTGGGCAACACCGACCGGCACAGCATCAAGAAGAACCTG
ATCGGAGCCCTGCTGTTCGACAGCGGCGAAACAGCCGAGGCCACCCGGCTGAAGAGAACC
GCCAGAAGAAGATACACCAGACGGAAGAACCGGATCTGCTATCTGCAAGAGATCTTCAGCA
ACGAGATGGCCAAGGTGGACGACAGCTTCTTCCACAGACTGGAAGAGTCCTTCCTGGTGGA
AGAGGATAAGAAGCACGAGCGGCACCCCATCTTCGGCAACATCGTGGACGAGGTGGCCTAC
CACGAGAAGTACCCCACCATCTACCACCTGAGAAAGAAACTGGTGGACAGCACCGACAAGG
CCGACCTGCGGCTGATCTATCTGGCCCTGGCCCACATGATCAAGTTCCGGGGCCACTTCCT
GATCGAGGGCGACCTGAACCCCGACAACAGCGACGTGGACAAGCTGTTCATCCAGCTGGTG
CAGACCTACAACCAGCTGTTCGAGGAAAACCCCATCAACGCCAGCGGCGTGGACGCCAAGG
CCATCCTGTCTGCCAGACTGAGCAAGAGCAGACGGCTGGAAAATCTGATCGCCCAGCTGCC
CGGCGAGAAGAAGAATGGCCTGTTCGGAAACCTGATTGCCCTGAGCCTGGGCCTGACCCCC
AACTTCAAGAGCAACTTCGACCTGGCCGAGGATGCCAAACTGCAGCTGAGCAAGGACACCT
ACGACGACGACCTGGACAACCTGCTGGCCCAGATCGGCGACCAGTACGCCGACCTGTTTCT
GGCCGCCAAGAACCTGTCCGACGCCATCCTGCTGAGCGACATCCTGAGAGTGAACACCGAG
ATCACCAAGGCCCCCCTGAGCGCCTCTATGATCAAGAGATACGACGAGCACCACCAGGACC
TGACCCTGCTGAAAGCTCTCGTGCGGCAGCAGCTGCCTGAGAAGTACAAAGAGATTTTCTT
CGACCAGAGCAAGAACGGCTACGCCGGCTACATTGACGGCGGAGCCAGCCAGGAAGAGTT
CTACAAGTTCATCAAGCCCATCCTGGAAAAGATGGACGGCACCGAGGAACTGCTCGTGAAG
CTGAACAGAGAGGACCTGCTGCGGAAGCAGCGGACCTTCGACAACGGCAGCATCCCCCACC
AGATCCACCTGGGAGAGCTGCACGCCATTCTGCGGCGGCAGGAAGATTTTTACCCATTCCT
GAAGGACAACCGGGAAAAGATCGAGAAGATCCTGACCTTCCGCATCCCCTACTACGTGGGC
CCTCTGGCCAGGGGAAACAGCAGATTCGCCTGGATGACCAGAAAGAGCGAGGAAACCATCA
CCCCCTGGAACTTCGAGGAAGTGGTGGACAAGGGCGCTTCCGCCCAGAGCTTCATCGAGCG
GATGACCAACTTCGATAAGAACCTGCCCAACGAGAAGGTGCTGCCCAAGCACAGCCTGCTG
TACGAGTACTTCACCGTGTATAACGAGCTGACCAAAGTGAAATACGTGACCGAGGGAATGA
GAAAGCCCGCCTTCCTGAGCGGCGAGCAGAAAAAGGCCATCGTGGACCTGCTGTTCAAGAC
CAACCGGAAAGTGACCGTGAAGCAGCTGAAAGAGGACTACTTCAAGAAAATCGAGTGCTTC
GACTCCGTGGAAATCTCCGGCGTGGAAGATCGGTTCAACGCCTCCCTGGGCACATACCACG
ATCTGCTGAAAATTATCAAGGACAAGGACTTCCTGGACAATGAGGAAAACGAGGACATTCT
GGAAGATATCGTGCTGACCCTGACACTGTTTGAGGACAGAGAGATGATCGAGGAACGGCTG
AAAACCTATGCCCACCTGTTCGACGACAAAGTGATGAAGCAGCTGAAGCGGCGGAGATACA
CCGGCTGGGGCAGGCTGAGCCGGAAGCTGATCAACGGCATCCGGGACAAGCAGTCCGGCA
AGACAATCCTGGATTTCCTGAAGTCCGACGGCTTCGCCAACAGAAACTTCATGCAGCTGAT
CCACGACGACAGCCTGACCTTTAAAGAGGACATCCAGAAAGCCCAGGTGTCCGGCCAGGGC
GATAGCCTGCACGAGCACATTGCCAATCTGGCCGGCAGCCCCGCCATTAAGAAGGGCATCC
TGCAGACAGTGAAGGTGGTGGACGAGCTCGTGAAAGTGATGGGCCGGCACAAGCCCGAGA
ACATCGTGATCGAAATGGCCAGAGAGAACCAGACCACCCAGAAGGGACAGAAGAACAGCC
GCGAGAGAATGAAGCGGATCGAAGAGGGCATCAAAGAGCTGGGCAGCCAGATCCTGAAAG
AACACCCCGTGGAAAACACCCAGCTGCAGAACGAGAAGCTGTACCTGTACTACCTGCAGAA
TGGGCGGGATATGTACGTGGACCAGGAACTGGACATCAACCGGCTGTCCGACTACGATGTG
GACGCTATCGTGCCTCAGAGCTTTCTGAAGGACGACTCCATCGACAACAAGGTGCTGACCA
GAAGCGACAAGAACCGGGGCAAGAGCGACAACGTGCCCTCCGAAGAGGTCGTGAAGAAGA
TGAAGAACTACTGGCGGCAGCTGCTGAACGCCAAGCTGATTACCCAGAGAAAGTTCGACAA
TCTGACCAAGGCCGAGAGAGGCGGCCTGAGCGAACTGGATAAGGCCGGCTTCATCAAGAG
ACAGCTGGTGGAAACCCGGCAGATCACAAAGCACGTGGCACAGATCCTGGACTCCCGGATG
AACACTAAGTACGACGAGAATGACAAGCTGATCCGGGAAGTGAAAGTGATCACCCTGAAGT
CCAAGCTGGTGTCCGATTTCCGGAAGGATTTCCAGTTTTACAAAGTGCGCGAGATCAACAA
CTACCACCACGCCCACGACGCCTACCTGAACGCCGTCGTGGGAACCGCCCTGATCAAAAAG
TACCCTAAGCTGGAAAGCGAGTTCGTGTACGGCGACTACAAGGTGTACGACGTGCGGAAGA
TGATCGCCAAGAGCGAGCAGGAAATCGGCAAGGCTACCGCCAAGTACTTCTTCTACAGCAA
CATCATGAACTTTTTCAAGACCGAGATTACCCTGGCCAACGGCGAGATCCGGAAGCGGCCT
CTGATCGAGACAAACGGCGAAACCGGGGAGATCGTGTGGGATAAGGGCCGGGATTTTGCC
ACCGTGCGGAAAGTGCTGAGCATGCCCCAAGTGAATATCGTGAAAAAGACCGAGGTGCAGA
CAGGCGGCTTCAGCAAAGAGTCTATCCTGCCCAAGAGGAACAGCGATAAGCTGATCGCCAG
AAAGAAGGACTGGGACCCTAAGAAGTACGGCGGCTTCGACAGCCCCACCGTGGCCTATTCT
GTGCTGGTGGTGGCCAAAGTGGAAAAGGGCAAGTCCAAGAAACTGAAGAGTGTGAAAGAG
CTGCTGGGGATCACCATCATGGAAAGAAGCAGCTTCGAGAAGAATCCCATCGACTTTCTGG
AAGCCAAGGGCTACAAAGAAGTGAAAAAGGACCTGATCATCAAGCTGCCTAAGTACTCCCT
GTTCGAGCTGGAAAACGGCCGGAAGAGAATGCTGGCCTCTGCCGGCGAACTGCAGAAGGG
AAACGAACTGGCCCTGCCCTCCAAATATGTGAACTTCCTGTACCTGGCCAGCCACTATGAG
AAGCTGAAGGGCTCCCCCGAGGATAATGAGCAGAAACAGCTGTTTGTGGAACAGCACAAGC
ACTACCTGGACGAGATCATCGAGCAGATCAGCGAGTTCTCCAAGAGAGTGATCCTGGCCGA
CGCTAATCTGGACAAAGTGCTGTCCGCCTACAACAAGCACCGGGATAAGCCCATCAGAGAG
CAGGCCGAGAATATCATCCACCTGTTTACCCTGACCAATCTGGGAGCCCCTGCCGCCTTCA
AGTACTTTGACACCACCATCGACCGGAAGAGGTACACCAGCACCAAAGAGGTGCTGGACGC
CACCCTGATCCACCAGAGCATCACCGGCCTGTACGAGACACGGATCGACCTGTCTCAGCTG

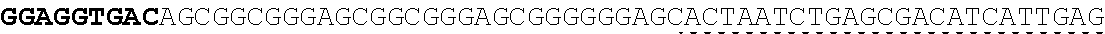

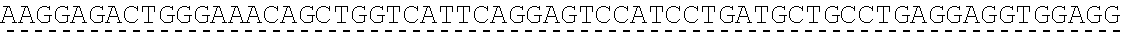

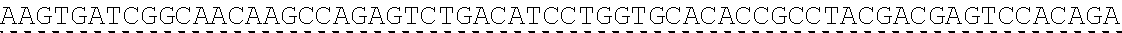

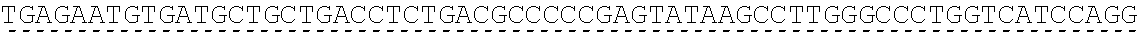

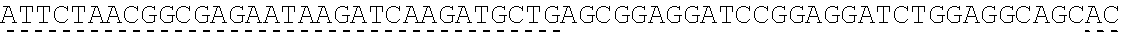

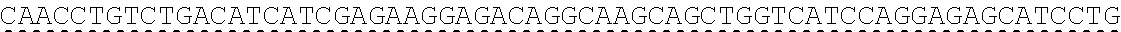

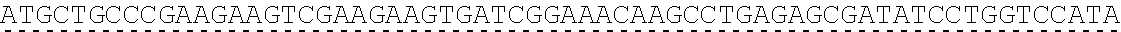

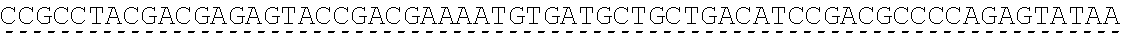

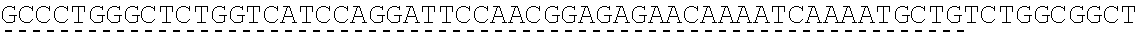

CAAAAAGAACCGCCGACGGCAGCGAATTCGAGCCCAAGAAGAAGAGGAAAGTC

### Split-DddAₜₒₓ-CCR5 TALE Fusions Sequences

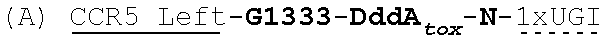

(SEQ ID NO: 342)
CCCAAGAAGAAGAGGAAGGTGGGCATTCACCGCGGGGTACCTATGGTGGACTTGAGGACACTCG
GTTATTCGCAACAGCAACAGGAGAAAATCAAGCCTAAGGTCAGGAGCACCGTCGCGCAACACCA
CGAGGCGCTTGTGGGGCATGGCTTCACTCATGCGCATATTGTCGCGCTTTCACAGCACCCTGCGG
CGCTTGGGACGGTGGCTGTCAAATACCAAGATATGATTGCGGCCCTGCCCGAAGCCACGCACGAG
GCAATTGTAGGGGTCGGTAAACAGTGGTCGGGAGCGCGAGCACTTGAGGCGCTGCTGACTGTGG
CGGGTGAGCTTAGGGGGCCTCCGCTCCAGCTCGACACCGGGCAGCTGCTGAAGATCGCGAAGAG
AGGGGGAGTAACAGCGGTAGAGGCAGTGCACGCCTGGCGCAATGCGCTCACCGGGGCCCCCTTG
AACCTGACCCCAGACCAGGTAGTCGCAATCGCGTCAAACGGAGGGGGAAAGCAAGCCCTGGAAA
CCGTGCAAAGGTTGTTGCCGGTCCTTTGTCAAGACCACGGCCTTACACCGGAGCAAGTCGTGGCC
ATTGCATCCCACGACGGTGGCAAACAGGCTCTTGAGACGGTTCAGAGACTTCTCCCAGTTCTCTG
TCAAGCCCACGGGCTGACTCCCGATCAAGTTGTAGCGATTGCGTCGAACATTGGAGGGAAACAA
GCATTGGAGACTGTCCAACGGCTCCTTCCCGTGTTGTGTCAAGCCCACGGTTTGACGCCTGCACA
AGTGGTCGCCATCGCCTCGAATGGCGGCGGTAAGCAGGCGCTGGAAACAGTACAGCGCCTGCTG
CCTGTACTGTGCCAGGATCATGGACTGACCCCAGACCAGGTAGTCGCAATCGCGTCAAACGGAGG
GGGAAAGCAAGCCCTGGAAACCGTGCAAAGGTTGTTGCCGGTCCTTTGTCAAGACCACGGCCTTA
CACCGGAGCAAGTCGTGGCCATTGCAAGCAACATCGGTGGCAAACAGGCTCTTGAGACGGTTCA
GAGACTTCTCCCAGTTCTCTGTCAAGCCCACGGGCTGACTCCCGATCAAGTTGTAGCGATTGCGTC
GCATGACGGAGGGAAACAAGCATTGGAGACTGTCCAACGGCTCCTTCCCGTGTTGTGTCAAGCCC
ACGGTTTGACGCCTGCACAAGTGGTCGCCATCGCCTCCAATATTGGCGGTAAGCAGGCGCTGGAA
ACAGTACAGCGCCTGCTGCCTGTACTGTGCCAGGATCATGGACTGACCCCAGACCAGGTAGTCGC
AATCGCGTCACATGACGGGGGAAAGCAAGCCCTGGAAACCGTGCAAAGGTTGTTGCCGGTCCTTT
GTCAAGACCACGGCCTTACACCGGAGCAAGTCGTGGCCATTGCATCCCACGACGGTGGCAAACA
GGCTCTTGAGACGGTTCAGAGACTTCTCCCAGTTCTCTGTCAAGCCCACGGGCTGACTCCCGATCA
AGTTGTAGCGATTGCGTCCAACGGTGGAGGGAAACAAGCATTGGAGACTGTCCAACGGCTCCTTC
CCGTGTTGTGTCAAGCCCACGGTTTGACGCCTGCACAAGTGGTCGCCATCGCCAACAACAACGGC
GGTAAGCAGGCGCTGGAAACAGTACAGCGCCTGCTGCCTGTACTGTGCCAGGATCATGGACTGAC
CCCAGACCAGGTAGTCGCAATCGCGTCACATGACGGGGGAAAGCAAGCCCTGGAAACCGTGCAA
AGGTTGTTGCCGGTCCTTTGTCAAGACCACGGCCTTACACCGGAGCAAGTCGTGGCCATTGCAAG
CAACATCGGTGGCAAACAGGCTCTTGAGACGGTTCAGAGACTTCTCCCAGTTCTCTGTCAAGCCC
ACGGGCTGACTCCCGATCAAGTTGTAGCGATTGCGAATAACAATGGAGGGAAACAAGCATTGGA
GACTGTCCAACGGCTCCTTCCCGTGTTGTGTCAAGCCCACGGTTTGACGCCTGCACAAGTGGTCGC
CATCGCCAGCCATGATGGCGGTAAGCAGGCGCTGGAAACAGTACAGCGCCTGCTGCCTGTACTGT
GCCAGGATCATGGACTGACACCCGAACAGGTGGTCGCCATTGCTTCTAATGGGGGAGGACGGCC
AGCCTTGGAGTCCATCGTAGCCCAATTGTCCAGGCCCGATCCCGCGTTGGCTGCGTTAACGAATG
ACCATCTGGTGGCGTTGGCATGTCTTGGTGGACGACCCGCGCTCGATGCAGTCAAAAAGGGTCTG
CCTCATGCTCCCGCATTGATCAAAAGAACCAACCGGCGGATTCCCGAGAGAACTTCCCATCGAGT
CGCGGGATCCGGCAGCTACGCCCTGGGTCCGTATCAGATTAGCGCCCCGCAGCTGCCAGCT
TACAATGGTCAGACCGTGGGTACCTTCTACTATGTGAACGACGCGGGCGGTCTGGAGAGCA

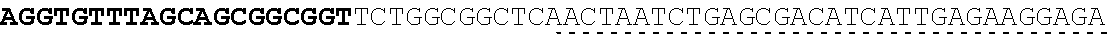

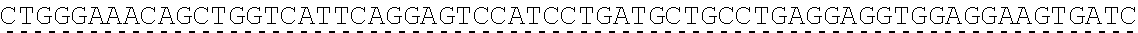

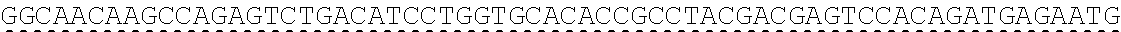

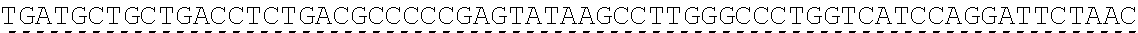

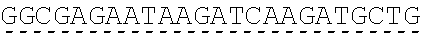

Tranlated amino acid sequence:
(SEQ ID NO: 343)
PKKKRKVGIHRGVPMVDLRTLGYSQQQQEKIKPKVRSTVAQHHEALVGHGFTHAHIVALSQHPAAL
GTVAVKYQDMIAALPEATHEAIVGVGKQWSGARALEALLTVAGELRGPPLQLDTGQLLKIAKRGGV
TAVEAVHAWRNALTGAPLNLTPDQVVAIASNGGGKQALETVQRLLPVLCQDHGLTPEQVVAIASHD
GGKQALETVQRLLPVLCQAHGLTPDQVVAIASNIGGKQALETVQRLLPVLCQAHGLTPAQVVAIASN
GGGKQALETVQRLLPVLCQDHGLTPDQVVAIASNGGGKQALETVQRLLPVLCQDHGLTPEQVVAIAS
NIGGKQALETVQRLLPVLCQAHGLTPDQVVAIASHDGGKQALETVQRLLPVLCQAHGLTPAQVVAIA
SNIGGKQALETVQRLLPVLCQDHGLTPDQVVAIASHDGGKQALETVQRLLPVLCQDHGLTPEQVVAI
ASHDGGKQALETVQRLLPVLCQAHGLTPDQVVAIASNGGGKQALETVQRLLPVLCQAHGLTPAQVV
AIANNNGGKQALETVQRLLPVLCQDHGLTPDQVVAIASHDGGKQALETVQRLLPVLCQDHGLTPEQV
VAIASNIGGKQALETVQRLLPVLCQAHGLTPDQVVAIANNNGGKQALETVQRLLPVLCQAHGLTPAQ
VVAIASHDGGKQALETVQRLLPVLCQDHGLTPEQVVAIASNGGGRPALESIVAQLSRPDPALAALTND
HLVALACLGGRPALDAVKKGLPHAPALIKRTNRRIPERTSHRVAGSGSYALGPYQISAPOLPAYNGQT
VGTFYYVNDAGGLESKVFSSGGSGGSTNLSDIIEKETGKQLVIQESILMLPEEVEEVIGNKPESDILVHT
AYDESTDENVMLLTSDAPEYKPWAL VIQDSNGENKIKML

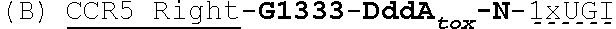

(SEQ ID NO: 344)
CCCAAGAAGAAGAGGAAGGTGGGCATTCACCGCGGGGTACCTATGGTGGACTTGAGGACACTCG
GTTATTCGCAACAGCAACAGGAGAAAATCAAGCCTAAGGTCAGGAGCACCGTCGCGCAACACCA
CGAGGCGCTTGTGGGGCATGGCTTCACTCATGCGCATATTGTCGCGCTTTCACAGCACCCTGCGG
CGCTTGGGACGGTGGCTGTCAAATACCAAGATATGATTGCGGCCCTGCCCGAAGCCACGCACGAG
GCAATTGTAGGGGTCGGTAAACAGTGGTCGGGAGCGCGAGCACTTGAGGCGCTGCTGACTGTGG
CGGGTGAGCTTAGGGGGCCTCCGCTCCAGCTCGACACCGGGCAGCTGCTGAAGATCGCGAAGAG
AGGGGGAGTAACAGCGGTAGAGGCAGTGCACGCCTGGCGCAATGCGCTCACCGGGGCCCCCTTG
AACCTGACCCCAGACCAGGTAGTCGCAATCGCGTCACATGACGGGGGAAAGCAAGCCCTGGAAA
CCGTGCAAAGGTTGTTGCCGGTCCTTTGTCAAGACCACGGCCTTACACCGGAGCAAGTCGTGGCC
ATTGCAAGCAATGGGGGTGGCAAACAGGCTCTTGAGACGGTTCAGAGACTTCTCCCAGTTCTCTG
TCAAGCCCACGGGCTGACTCCCGATCAAGTTGTAGCGATTGCGTCCAACGGTGGAGGGAAACAA
GCATTGGAGACTGTCCAACGGCTCCTTCCCGTGTTGTGTCAAGCCCACGGTTTGACGCCTGCACA
AGTGGTCGCCATCGCCAGCCATGATGGCGGTAAGCAGGCGCTGGAAACAGTACAGCGCCTGCTG
CCTGTACTGTGCCAGGATCATGGACTGACCCCAGACCAGGTAGTCGCAATCGCGTCACATGACGG
GGGAAAGCAAGCCCTGGAAACCGTGCAAAGGTTGTTGCCGGTCCTTTGTCAAGACCACGGCCTTA
CACCGGAGCAAGTCGTGGCCATTGCAAGCAACATCGGTGGCAAACAGGCTCTTGAGACGGTTCA
GAGACTTCTCCCAGTTCTCTGTCAAGCCCACGGGCTGACTCCCGATCAAGTTGTAGCGATTGCGA
ATAACAATGGAGGGAAACAAGCATTGGAGACTGTCCAACGGCTCCTTCCCGTGTTGTGTCAAGCC
CACGGTTTGACGCCTGCACAAGTGGTCGCCATCGCCTCCAATATTGGCGGTAAGCAGGCGCTGGA
AACAGTACAGCGCCTGCTGCCTGTACTGTGCCAGGATCATGGACTGACCCCAGACCAGGTAGTCG
CAATCGCGTCGAACATTGGGGGAAAGCAAGCCCTGGAAACCGTGCAAAGGTTGTTGCCGGTCCTT
TGTCAAGACCACGGCCTTACACCGGAGCAAGTCGTGGCCATTGCAAGCAATGGGGGTGGCAAAC
AGGCTCTTGAGACGGTTCAGAGACTTCTCCCAGTTCTCTGTCAAGCCCACGGGCTGACTCCCGATC
AAGTTGTAGCGATTGCGTCCAACGGTGGAGGGAAACAAGCATTGGAGACTGTCCAACGGCTCCTT
CCCGTGTTGTGTCAAGCCCACGGTTTGACGCCTGCACAAGTGGTCGCCATCGCCAACAACAACGG
CGGTAAGCAGGCGCTGGAAACAGTACAGCGCCTGCTGCCTGTACTGTGCCAGGATCATGGACTGA
CCCCAGACCAGGTAGTCGCAATCGCGTCGAACATTGGGGGAAAGCAAGCCCTGGAAACCGTGCA
AAGGTTGTTGCCGGTCCTTTGTCAAGACCACGGCCTTACACCGGAGCAAGTCGTGGCCATTGCAA
GCAATGGGGGTGGCAAACAGGCTCTTGAGACGGTTCAGAGACTTCTCCCAGTTCTCTGTCAAGCC
CACGGGCTGACTCCCGATCAAGTTGTAGCGATTGCGTCGAACATTGGAGGGAAACAAGCATTGG
AGACTGTCCAACGGCTCCTTCCCGTGTTGTGTCAAGCCCACGGTTTGACGCCTGCACAAGTGGTC
GCCATCGCCAGCCATGATGGCGGTAAGCAGGCGCTGGAAACAGTACAGCGCCTGCTGCCTGTACT
GTGCCAGGATCATGGACTGACACCCGAACAGGTGGTCGCCATTGCTTCTAATGGGGGAGGACGG
CCAGCCTTGGAGTCCATCGTAGCCCAATTGTCCAGGCCCGATCCCGCGTTGGCTGCGTTAACGAA
TGACCATCTGGTGGCGTTGGCATGTCTTGGTGGACGACCCGCGCTCGATGCAGTCAAAAAGGGTC
TGCCTCATGCTCCCGCATTGATCAAAAGAACCAACCGGCGGATTCCCGAGAGAACTTCCCATCGA
GTCGCGGGATCCCCAACCCCGTACCCAAACTATGCCAATGCCGGTCATGTGGAGGGTCAGAG
CGCCCTGTTCATGCGTGATAACGGCATCAGCGAGGGTCTGGTGTTCCACAACAACCCGGAA
GGCACCTGCGGTTTTTGCGTGAACATGACCGAGACCCTGCTGCCGGAAAACGCGAAAATGA
CCGTGGTGCCGCCGGAAGGTGCCATTCCAGTGAAGCGCGGCGCTACCGGTGAAACCAAAG

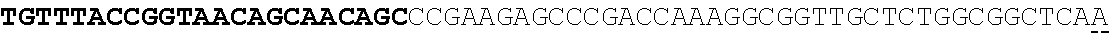

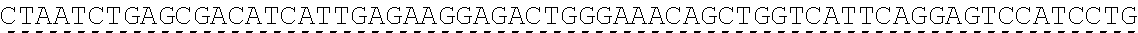

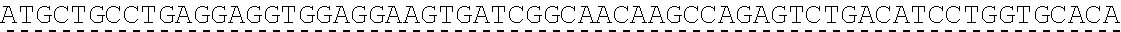

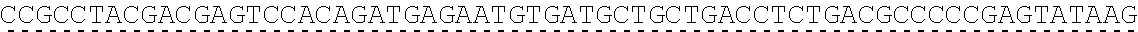

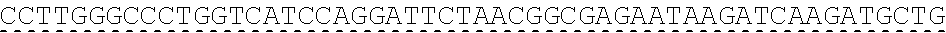

Translated amino acid sequence:
(SEQ ID NO: 345)
PKKKRKVGIHRGVPMVDLRTLGYSQQQQEKIKPKVRSTVAQHHEALVGHGFTHAHIVALSQHPAAL
GTVAVKYQDMIAALPEATHEAIVGVGKQWSGARALEALLTVAGELRGPPLQLDTGQLLKIAKRGGV
TAVEAVHAWRNALTGAPLNLTPDQVVAIASHDGGKQALETVQRLLPVLCQDHGLTPEQVVAIASNG
GGKQALETVQRLLPVLCQAHGLTPDQVVAIASNGGGKQALETVQRLLPVLCQAHGLTPAQVVAIASH
DGGKQALETVQRLLPVLCQDHGLTPDQVVAIASHDGGKQALETVQRLLPVLCQDHGLTPEQVVAIAS
NIGGKQALETVQRLLPVLCQAHGLTPDQVVAIANNNGGKQALETVQRLLPVLCQAHGLTPAQVVAIA
SNIGGKQALETVQRLLPVLCQDHGLTPDQVVAIASNIGGKQALETVQRLLPVLCQDHGLTPEQVVAIA
SNGGGKQALETVQRLLPVLCQAHGLTPDQVVAIASNGGGKQALETVQRLLPVLCQAHGLTPAQVVAI
ANNNGGKQALETVQRLLPVLCQDHGLTPDQVVAIASNIGGKQALETVQRLLPVLCQDHGLTPEQVVA
IASNGGGKQALETVQRLLPVLCQAHGLTPDQVVAIASNIGGKQALETVQRLLPVLCQAHGLTPAQVV
AIASHDGGKQALETVQRLLPVLCQDHGLTPEQVVAIASNGGGRPALESIVAQLSRPDPALAALTNDHL
VALACLGGRPALDAVKKGLPHAPALIKRTNRRIPERTSHRVAGSPTPYPNYANAGHVEGQSALFMRD
NGISEGLVFHNNPEGTCGFCVNMTETLLPENAKMTVVPPEGAIPVKRGATGETKVFTGNSNSPKSPTK
GGCSGGSTNLSDIIEKETGKQLVIQESILMLPEEVEEVIGNKPESDILVHTAYDESTDENVMLLTSDAPE
YKPWALVIQDSNGENKIKML

General DdCBE Architecture and mitoTALE Sequences

### Legend:

| | |
|---|---|
| COX8A MTS | COX8A MTS (Right)-Underlined |
| SOD2 MTS | SOD2 MTS (Left)-Bolded |
| 3xFLAG | [symbol] -Dashed Underlined |
| 3xHA | 3xHA (Left)-Italicized and Underlined |
| mitoTALE | mitoTALE-Bolded and Heavy Underlined |
| DddAₜₒₓ half | [symbol] -Bolded, Italicized, and Underlined |
| 1x-UGI1x-UGI | [symbol] -Thick Dashed Underlined |
| ATP5B 3′UTR | [symbol] -Dotted Underlined |
| SOD2 3′UTR | SOD2 3UTR (Left)-Double Underlined |

All right-side halves of DdCBEs have the general architecture of (from N- to C-terminus): COX8A MTS-3×FLAG-mitoTALE-2aa linker-DddAₜₒₓ half-4aa linker-1×-UGI-ATP5B 3′UTR

All left-side halves of DdCBEs have the general architecture of (from N- to C-terminus): SOD2 MTS-3×HA-mitoTALE-2aa linker-DddAₜₒₓ half-4aa linker-1×-UGI-SOD2 3′UTR

(A) SOD2 MTS
(SEQ ID NO: 13)
MLSRAVCGTSRQLAPVLGYLGSRQKHSLPD
(B) COX8A MTS
(SEQ ID NO: 14)
MSVLTPLLLRGLTGSARRLPVPRAKIHSL
(C) SOD2 3′UTR
(SEQ ID NO: 15)
ACCACGATCGTTATGCTGATCATACCCTAATGATCCCAGCAAGATAATGTCCTGTCTTCTAAGATG
TGCATCAAGCCTGGTACATACTGAAAACCCTATAAGGTCCTGGATAATTTTTGTTTGATTATTCAT
TGAAGAAACATTTATTTTCCAATTGTGTGAAGTTTTTGACTGTTAATAAAAGAATCTGTCAACCAT
CAAAAAAAAAAAAAAA
(D) ATP5B 3′UTR
(SEQ ID NO: 15)
ACCACGATCGTTATGCTGATCATACCCTAATGATCCCAGCAAGATAATGTCCTGTCTTCTAAGATG
TGCATCAAGCCTGGTACATACTGAAAACCCTATAAGGTCCTGGATAATTTTTGTTTGATTATTCAT
TGAAGAAACATTTATTTTCCAATTGTGTGAAGTTTTTGACTGTTAATAAAAGAATCTGTCAACCAT
CAAAAAAAAAAAAAAA
(E) ND6-DdCBE: Left mitoTALE-G1397-DddAₜₒₓ₋N-1x-UGI
(SEQ ID NO: 144)
ATGGCCCTGTCCCGTGCGGTTTGTGGCACCTCCCGTCAACTGGCTCCGGTTCTGGGTTATC
TGGGTTCCCGTCAAAAACACTCCCTGCCGGACTACCCGTATGATGTTCCGGATTACGCTGGCTAC
CCATACGACGTCCCAGACTACGCTGGCTACCCATACGACGTCCCAGACTACGCTATGGACATCGCGG
ATCTGCGTACCCTGGGTTACAGCCAGCAGCAGCAGGAGAAGATCAAGCCGAAGGTGCGCA
GCACCGTGGCTCAGCACCACGAAGCCCTGGTGGGCCACGGTTTCACCCACGCTCACATTGT
GGCCCTGAGCCAGCACCCAGCCGCGCTGGGCACCGTGGCCGTGAAATATCAGGATATGATT
GCTGCCCTGCCAGAGGCCACCCATGAAGCTATTGTGGGCGTGGGCAAGCAGTGGAGCGGT
GCTCGTGCGCTGGAGGCGCTGCTGACCGTGGCTGGTGAACTGCGTGGTCCGCCGCTGCAG
CTGGACACCGGTCAGCTGCTGAAAATCGCGAAACGTGGCGGTGTGACCGCGGTGGAAGCC
GTGCATGCTTGGCGTAATGCTCTGACCGGTGCGCCGCTGAACCTGACCCCGCAGCAGGTGG
TGGCTATTGCCAGCAACAACGGCGGTAAACAGGCCCTGGAGACCGTGCAGCGCCTGCTGCC
GGTGCTGTGCCAGGCCCATGGTCTGACCCCGGAGCAGGTGGTGGCGATCGCTAGCAACATC
GGCGGCAAGCAGGCCCTGGAAACCGTGCAGGCGCTGTTACCGGTGCTGTGCCAGGCTCAT
GGCCTGACCCCGGAACAAGTIGTGGCTATTGCCAGCCATGATGGCGGTAAACAGGCTCTGG
AAACCGTGCAGCGTCTGTTGCCGGTGCTGTGCCAAGCCCATGGCCTGACCCCGGAGCAAGT
TGTGGCTATTGCGAGCCATGATGGCGGCAAGCAGGCGCTGGAAACCGTTCAGCGCCTGTTA
CCGGTGCTGTGCCAAGCTCATGGTCTGACCCCGGAACAGGTGGTGGCCATTGCTTCCCATG
ATGGCGGTAAACAGGCCCTGGAAACCGTTCAGCGTCTGCTTCCGGTGCTGTGCCAGGCCCA
TGGGCTGACCCCGGAACAAGTGGTTGCTATTGCCAGCCACGATGGCGGCAAGCAGGCTCTG
GAGACCGTTCAGCGCCTGCTTCCGGTGCTGTGCCAGGCCCATGGCTTAACCCCGGAACAAG
TTGTTGCTATTGCTAGTCATGATGGCGGTAAACAGGCGCTGGAGACCGTTCAGCGTCTGTT
ACCGGTGCTGTGCCAGGCGCATGGCTTAACCCCGGAGCAGGTTGTTGCCATTGCCTCCAAT
ATCGGCGGCAAGCAGGCTCTGGAAACCGTTCAGGCCCTGTTGCCGGTGCTGTGCCAGGCCC
ATGGACTGACCCCGCAGCAAGTTGTTGCCATTGCCAGCAATGGCGGTGGCAAACAGGCGCT
GGAAACTGTTCAGCGCCTGCTCCCGGTGCTGTGCCAAGCGCATGGTCTGACCCCGCAGCAA
GTGGTTGCTATTGCTAGCAATGGTGGCGGTCGTCCGGCGCTGGAAAGCATTGTGGCTCAGC
TGAGCCGTCCAGACCCGGCCCTGGCGGCTCTGACCAACGATCACCTGGTGGCGCTGGCTTG
CCTGGGCGGTCGTCCGGCCCTGGATGCGGTGAAGAAAGGCCTGGGTGGATCCGGCAGCTAC
GCCCTGGGTCCGTATCAGATTAGCGCCCCGCAGCTGCCAGCTTACAATGGTCAGACCGTGGGTACC
TTCTACTATGTGAACGACGCGGGCGGTCTGGAGAGCAAGGTGTTTAGCAGCGGCGGTCCAACCCCG
TACCCAAACTATGCCAATGCCGGTCATGTGGAGGGTCAGAGCGCCCTGTTCATGCGTGATAACGGC
ATCAGCGAGGGTCTGGTGTTCCACAACAACCCGGAAGGCACCTGCGGTTTTTGCGTGAACATGACC

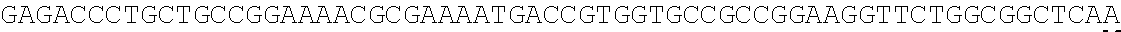

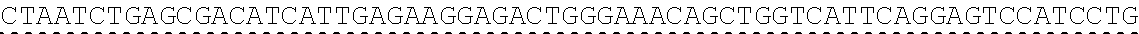

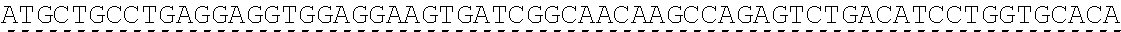

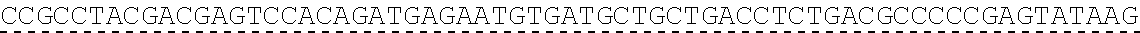

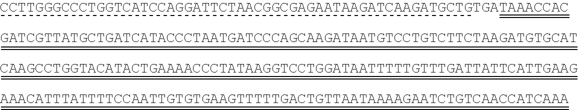

(F) ND6-DdCBE: Right mitoTALE-G1397-DddAₜₒₓ₋N-1x-UGI
TCCGTTCTGACCCCGCTGCTGCTGCGTGGCCTGACCGGCTCCGCTCGTCGTCTGCCAGTTCCGCGT

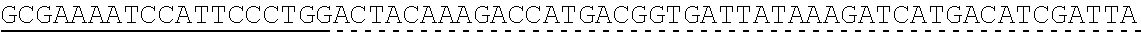

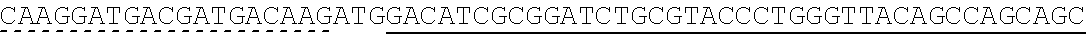

AGCAGGAGAAGATCAAGCCAAAGGTGCGCAGCACCGTGGCCCAGCACCATGAAGCTCTGG
TGGGTCACGGCTTCACCCACGCGCACATCGTGGCTCTGAGCCAGCACCCAGCCGCGCTGGG
ATTGTGGGTGTGGGCAAGAGAGGAGCCGGTGCTCGTCTGCGCTGGAGGCCCTGCTGACCGTG
GCCGGTGAACTGCGTGGCCCGCCGCTGCAGCTGGATACCGGCCAGCTGCTGAAAATCGCG
AAACGTGGCGGTGTGACCGCTGTGGAAGCTGTGCATGCCTGGCGTAATGCTCTGACCGGTG
CCCCGCTGAACCTGACCCCGCAGCAGGTGGTGGCTATTGCCAGCAACAACGGCGGTAAACA
GAGCAGGTGGTGGCGATCGCTAGCAACATCGGCGGCAAGCAGGCTCTGGAGACCGTTCAG
CCAGCAATGGCGGTGGCAAACAGGCGCTGGAGACCGTGCAGCGTCTGTTGCCGGTGCTGT
GCCAAGCCCATGGGCTGACCCCGCAGCAAGTGGTTGCCATCGCCAGCAACAACGGTGGCAA
GCAGGCCCTGGAGACCGTTCAGCGCCTGTTACCGGTGCTGTGCCAGGCCCATGGCTTAACC
CCGCAGCAAGTTGTGGCCATCGCTAGCAACAACGGTGGCAAACAGGCTCTGGAGACTGTTC
AGCGTCTGCTTCCGGTGCTGTGCCAAGCGCATGGCCTGACCCCGGAACAAGTTGTTGCTAT
TGCCAGCCATGATGGTGGCAAGCAGGCGCTGGAAACCGTTCAGCGCCTGCTTCCGGTGCTG
TGCCAGGCGCATGGATTAACCCCGCAGCAAGTGGTGGCCATCGCCAGCAATGGTGGCGGTA
AACAGGCCCTGGAAACCGTTCAGCGTCTGTTACCGGTGCTGTGCCAGGCCCATGGATTAAC
CCCGGAACAAGTTGTGGCTATTGCGTCCAATATCGGCGGCAAGCAGGCGCTGGAAACTGTG
CAGGCTCTGCTCCCGGCCTGTGCCAGGCCCATGGGTTAACCCCGCAGCAGGTTGTTGCCA
TTGCGAGCAACGGCGGTGGCAAACAGGCTCTGGAGACGGTTCAGCGCCTGCTCCCGTGCT
GTGCCAGGCCCATGGTTTAACCCCGCAGCAGGTGGTTGCTATTGCTAGCAATGGCGGCGGC
AAGCAGGCGCTGGAAACGGTGCAGCGTCTGCTACCGGTGCTGTGCCAGGCACATGGCCTTA
CCCCGCAGCAAGTTGTGGCCATTGCTAGCAATGGCGGTGGCCGTCCGGCCCTGGAAAAGCAT
TGTGGCGCAGCTGAGCCGTCCAGACCCGGCCCTGGCGGCTCTGACCAATGATCACCTGGTG
GCCCTGGCCTGCCTGGGTGGCCGTCCGGCTCTGGATGCCGTGAAGAAAGGTCTGGGCGGAT
CCGCCATTCCAGTGAAGCGCGGCGCTACCGGTGAAACCAAAGTGTTTACCGGTAACAGCAACAGCC

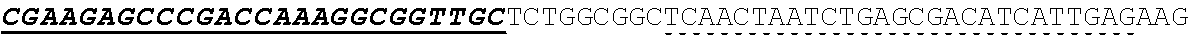

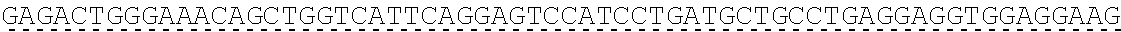

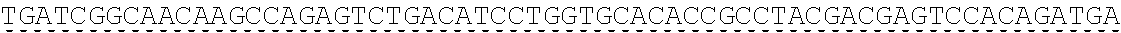

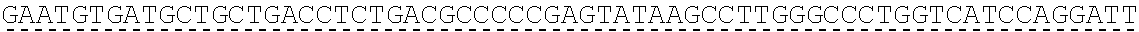

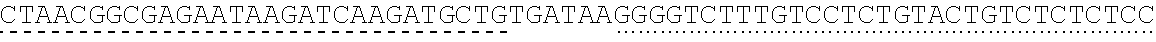

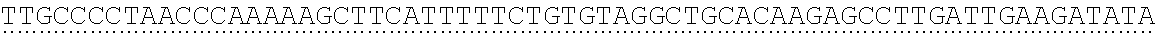

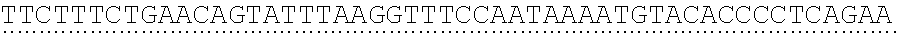

(G) ND1-DdCBE Right mitoTALE repeat
(SEQ ID NO: 146)
GACATCGCGGATCTGCGTACCCTGGGTTACAGCCAGCAGCAGCAGGAGAAGATCAAGCCAAAGG
TGCGCAGCACCGTGGCCCAGCACCATGAAGCTCTGGTGGGTCACGGCTTCACCCACGCGCACATC
GTGGCTCTGAGCCAGCACCCAGCCGCGCTGGGTACCGTGGCCGTGAAATATCAGGACATGATTGC
TGCCCTGCCAGAGGCTACCCATGAAGCGATTGTGGGTGTGGGCAAGCAGTGGAGCGGTGCTCGTG
CGCTGGAGGCCCTGCTGACCGTGGCCGGTGAACTGCGTGGCCCGCCGCTGCAGCTGGATACCGGC
CAGCTGCTGAAAATCGCGAAACGTGGCGGTGTGACCGCTGTGGAAGCTGTGCATGCCTGGCGTAA
TGCTCTGACCGGTGCCCCGCTGAACCTGACCCCGGAACAGGTGGTTGCCATCGCATCCAATAATG
GTGGTAAACAAGCTCTGGAGACCGTTCAAGCCCTGCTGCCAGTGCTGTGCCAGGCTCATGGTCTG
ACCCCGCAGCAAGTTGTGGCTATTGCCAGCAACATCGGCGGCAAGCAGGCCCTGGAGACCGTGC
AGCGTCTGCTGCCGGTGCTGTGCCAGGCCCATGGCCTGACCCCGCAGCAAGTGGTTGCTATCGCC
AGCAACAACGGCGGTAAACAGGCTCTGGAAACCGTGCAGCGCCTGTTACCGGTGCTGTGCCAAG
CCCATGGTCTGACCCCGGAGCAGGTGGTGGCGATTGCTAGCAACGGCGGTGGCAAGCAGGCTCT
GGAGACCGTTCAGGCCCTGCTTCCGGTGCTGTGCCAAGCGCATGGCCTGACCCCGGAACAAGTTG
TTGCCATTGCCAGCAATGGTGGCGGTAAACAGGCGCTGGAAACCGTGCAGGCTCTGTTACCGGTG
CTGTGCCAGGCCCATGGGCTGACCCCGGAGCAAGTGGTGGCTATTGCGAGCAATGGCGGTGGCA
AGCAGGCCCTGGAAACCGTGCAGGCGCTGTTGCCGGTGCTGTGCCAAGCCCATGGATTAACCCCG
GAACAAGTGGTGGCGATCGCTAGCAACAACGGTGGCAAACAGGCGCTGGAGACCGTTCAGCGTC
TGTTACCGGTGCTGTGCCAGGCGCATGGCTTAACCCCGGAACAGGTTGTTGCGATTGCCAGCAAC
ATTGGTGGCAAGCAGGCTCTGGAAACCGTTCAGGCCCTGCTCCCGGTGCTGTGCCAGGCCCATGG
TTTAACCCCGGAACAGGTGGTGGCCATTGCCAGCAACGGTGGCGGTAAACAGGCCCTGGAGACC
GTTCAGCGCCTGCTACCGGTGCTGTGCCAGGCCCATGGACTGACCCCGGAGCAAGTTGTTGCCAT
TGCTAGCAACAACGGCGGCAAGCAGGCGCTGGAGACCGTGCAGGCTCTGCTTCCGGTGCTGTGCC
AGGCCCATGGGTTAACCCCGGAGCAGGTTGTGGCCATCGCCAGCCACGACGGCGGTAAACAGGC
CCTGGAAACCGTTCAGGCGCTGCTACCGGTGCTGTGCCAGGCACATGGCTTAACCCCGGAGCAGG
TGGTTGCCATCGCCTCCAATGGCGGTGGCAAGCAGGCTCTGGAAACGGTGCAGGCCCTGCTGCCG
GTGCTGTGCCAAGCCCATGGGTTGACCCCGGAACAAGTGGTGGCTATTGCTAGCCACGACGGTGG
CAAACAGGCTCTGGAGACTGTTCAGCGTCTGCTTCCGGTGCTGTGCCAGGCTCATGGCTTAACCC
CGCAGCAAGTTGTTGCTATTGCCTCCAATATTGGTGGCAAGCAGGCGCTGGAAACCGTTCAGCGC
CTGCTGCCGGTGCTGTGCCAGGCTCATGGGCTTACCCCGGAACAAGTTGTGGCCATTGCCTCCCAT
GATGGTGGCAAACAGGCGCTGGAAACTGTGCAGGCTCTGCTCCCGGTGCTGTGCCAGGCTCATGG
ATTAACCCCGCAGCAAGTGGTGGCCATTGCTAGCCACGATGGTGGCAAGCAGGCCCTGGAGACG
GTTCAGCGTCTGCTCCCGGTGCTGTGCCAGGCCCATGGGCTAACCCCGCAGCAGGTTGTTGCTATT
GCCAGTCATGATGGTGGCAAACAGGCTCTGGAAACTGTGCAGCGCCTGCTACCGGTGCTGTGCCA
GGCTCACGGTCTGACCCCGCAGCAGGTGGTGGCAATCGCAAGCAACGGTGGTGGTCGTCCGGCA
CTGGAAAGCATTGTGGCGCAGCTGAGCCGTCCAGACCCGGCCCTGGCGGCTCTGACCAATGATCA
CCTGGTGGCCCTGGCCTGCCTGGGTGGCCGTCCGGCTCTGGATGCCGTGAAGAAAGGTCTGGGC
Translated amino acid sequence:
(SEQ ID NO: 147)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASNNGGKQALETVQALLPVLCQAHGLTPQQVVAIASNIGGKQALETVQRLLPVL
CQAHGLTPQQVVAIASNNGGKQALETVQRLLPVLCQAHGLTPEQVVAIASNGGGKQALETVQALLPV
LCQAHGLTPEQVVAIASNGGGKQALETVQALLPVLCQAHGLTPEQVVAIASNGGGKQALETVQALLP
VLCQAHGLTPEQVVAIASNNGGKQALETVQRLLPVLCQAHGLTPEQVVAIASNIGGKQALETVQALL
PVLCQAHGLTPEQVVAIASNGGGKQALETVQRLLPVLCQAHGLTPEQVVAIASNNGGKQALETVQAL
LPVLCQAHGLTPEQVVAIASHDGGKQALETVQALLPVLCQAHGLTPEQVVAIASNGGGKQALETVQA
LLPVLCQAHGLTPEQVVAIASHDGGKQALETVQRLLPVLCQAHGLTPQQVVAIASNIGGKQALETVQ
RLLPVLCQAHGLTPEQVVAIASHDGGKQALETVQALLPVLCQAHGLTPQQVVAIASHDGGKQALETV
QRLLPVLCQAHGLTPQQVVAIASHDGGKQALETVQRLLPVLCQAHGLTPQQVVAIASNGGGRPALESI
VAQLSRPDPALAALTNDHLVALACLGGRPALDAVKKGLG
(H) ND1-DdCBE Left mitoTALE repeat
(SEQ ID NO: 148)
GACATCGCGGATCTGCGTACCCTGGGTTACAGCCAGCAGCAGCAGGAGAAGATCAAGCCGAAGG
TGCGCAGCACCGTGGCTCAGCACCACGAAGCCCTGGTGGGCCACGGTTTCACCCACGCTCACATT
GTGGCCCTGAGCCAGCACCCAGCCGCGCTGGGCACCGTGGCCGTGAAATATCAGGATATGATTGC
TGCCCTGCCAGAGGCCACCCATGAAGCTATTGTGGGCGTGGGCAAGCAGTGGAGCGGTGCTCGTG
CGCTGGAGGCGCTGCTGACCGTGGCTGGTGAACTGCGTGGTCCGCCGCTGCAGCTGGACACCGGT
CAGCTGCTGAAAATCGCGAAACGTGGCGGTGTGACCGCGGTGGAAGCCGTGCATGCTTGGCGTA
ATGCTCTGACCGGTGCGCCGCTGAACCTGACCCCGGAACAAGTGGTTGCTATCGCATCCCATGAC
GGCGGTAAACAAGCCCTGGAGACCGTTCAAGCCCTGCTGCCAGTGCTGTGCCAGGCTCATGGTCT
GACCCCGCAGCAGGTGGTGGCTATTGCCAGCAATGGCGGTGGCAAGCAGGCGCTGGAGACCGTG
CAGCGTCTGCTGCCGGTGCTGTGCCAAGCCCATGGCCTGACCCCGCAGCAAGTTGTGGCTATCGC
CAGCAACATTGGTGGCAAACAGGCCCTGGAAACCGTGCAGCGCCTGTTACCGGTGCTGTGCCAGG
CCCATGGTCTGACCCCGGAGCAGGTGGTGGCGATCGCTAGCAACAACGGTGGCAAGCAGGCTCT
GGAAACCGTGCAGGCCCTGCTTCCGGTGCTGTGCCAGGCCCATGGGCTGACCCCGGAACAAGTTG
TGGCTATTGCCAGCCACGACGGTGGCAAACAGGCGCTGGAAACCGTGCAGGCTCTGTTACCGGTG
CTGTGCCAAGCGCATGGCCTGACCCCGGAACAGGTGGTGGCTATTGCTAGCCACGATGGTGGCAA
GCAGGCCCTGGAGACCGTTCAGGCGCTGTTGCCGGTGCTGTGCCAGGCGCATGGCTTAACCCCGG
AACAAGTTGTTGCGATTGCTAGCAACGGTGGCGGTAAACAGGCTCTGGAGACCGTTCAGCGTCTG
TTACCGGTGCTGTGCCAGGCACATGGCCTGACCCCGGAGCAAGTTGTTGCCATTGCCAGCAACAT
CGGCGGCAAGCAGGCTCTGGAGACCGTGCAGGCCCTGCTCCCGGTGCTGTGCCAGGCCCATGGCT
TAACCCCGGAGCAAGTTGTGGCCATTGCCAGCAACAACGGCGGTAAACAGGCGCTGGAGACCGT
TCAGCGCCTGCTACCGGTGCTGTGCCAGGCTCATGGCTTGACCCCGGAACAGGTTGTTGCGATTG
CGAGCCATGATGGCGGCAAGCAGGCGCTGGAAACCGTTCAGGCTCTGCTTCCGGTGCTGTGCCAG
GCCCATGGATTAACCCCGGAGCAGGTTGTTGCTATTGCCAGCCATGATGGCGGTAAACAGGCCCT
GGAGACCGTGCAGGCGCTGCTACCGGTGCTGTGCCAGGCTCATGGGCTGACCCCGGAGCAAGTG
GTTGCTATCGCGAGCAACAATGGCGGCAAGCAGGCTCTGGAAACGGTGCAGGCCCTGCTGCCGG
TGCTGTGCCAGGCCCATGGGTTAACCCCGGAACAAGTGGTGGCCATCGCTAGCAACGGCGGTGGC
AAACAGGCCCTGGAGACTGTTCAGCGTCTGCTTCCGGTGCTGTGCCAGGCCCATGGGCTAACCCC
GCAGCAAGTGGTTGCCATTGCCAGCAATGGCGGCGGCAAGCAGGCTCTGGAAACTGTGCAGCGC
CTGCTGCCGGTGCTGTGCCAGGCTCACGGTCTGACCCCGCAACAGGTGGTGGCAATCGCAAGCAA
TGGTGGTGGTCGTCCGGCACTGGAGAGCATTGTGGCTCAGCTGAGCCGTCCAGACCCGGCCCTGG
CGGCTCTGACCAACGATCACCTGGTGGCGCTGGCTTGCCTGGGCGGTCGTCCGGCCCTGGATGCG
GTGAAGAAAGGCCTGGGT
Translated amino acid sequence:
(SEQ ID NO: 149)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASHDGGKQALETVQALLPVLCQAHGLTPQQVVAIASNGGGKQALETVQRLLPV
LCQAHGLTPQQVVAIASNIGGKQALETVQRLLPVLCQAHGLTPEQVVAIASNNGGKQALETVQALLP
VLCQAHGLTPEQVVAIASHDGGKQALETVQALLPVLCQAHGLTPEQVVAIASHDGGKQALETVQALL
PVLCQAHGLTPEQVVAIASNGGGKQALETVQRLLPVLCQAHGLTPEQVVAIASNIGGKQALETVQAL
LPVLCQAHGLTPEQVVAIASNNGGKQALETVQRLLPVLCQAHGLTPEQVVAIASHDGGKQALETVQA
LLPVLCQAHGLTPEQVVAIASHDGGKQALETVQALLPVLCQAHGLTPEQVVAIASNNGGKQALETVQ
ALLPVLCQAHGLTPEQVVAIASNGGGKQALETVQRLLPVLCQAHGLTPQQVVAIASNGGGKQALETV
QRLLPVLCQAHGLTPQQVVAIASNGGGRPALESIVAQLSRPDPALAALTNDHLVALACLGGRPALDA
VKKGLG
(I) ND2-DdCBE Right mitoTALE repeat
(SEQ ID NO: 150)
GACATCGCGGATCTGCGTACCCTGGGTTACAGCCAGCAGCAGCAGGAGAAGATCAAGCCAAAGG
TGCGCAGCACCGTGGCCCAGCACCATGAAGCTCTGGTGGGTCACGGCTTCACCCACGCGCACATC
GTGGCTCTGAGCCAGCACCCAGCCGCGCTGGGTACCGTGGCCGTGAAATATCAGGACATGATTGC
TGCCCTGCCAGAGGCTACCCATGAAGCGATTGTGGGTGTGGGCAAGCAGTGGAGCGGTGCTCGTG
CGCTGGAGGCCCTGCTGACCGTGGCCGGTGAACTGCGTGGCCCGCCGCTGCAGCTGGATACCGGC
CAGCTGCTGAAAATCGCGAAACGTGGCGGTGTGACCGCTGTGGAAGCTGTGCATGCCTGGCGTAA
TGCTCTGACCGGTGCCCCGCTGAACCTGACCCCGGAACAAGTGGTTGCCATCGCATCCAATATCG
GTGGTAAACAAGCCCTGGAGACCGTTCAAGCCCTGCTGCCAGTGCTGTGCCAGGCTCATGGTCTG
ACCCCGCAGCAGGTGGTGGCCATTGCGAGCAACAATGGCGGCAAGCAGGCGCTGGAGACCGTGC
AGCGTCTGCTGCCGGTGCTGTGCCAAGCCCATGGCCTGACCCCGCAGCAAGTGGTTGCTATCGCC
AGCAACATTGGCGGTAAACAGGCCCTGGAAACCGTGCAGCGCCTGTTACCGGTGCTGTGCCAGGC
CCATGGTCTGACCCCGGAGCAGGTGGTGGCGATCGCTAGCAACATCGGCGGCAAGCAGGCTCTG
GAAACCGTGCAGGCCCTGCTTCCGGTGCTGTGCCAGGCCCATGGGCTGACCCCGGAACAAGTTGT
GGCTATTGCCAGCCATGATGGCGGTAAACAGGCGCTGGAAACCGTGCAGGCTCTGTTACCGGTGC
TGTGCCAAGCGCATGGCCTGACCCCGGAACAGGTGGTGGCTATTGCGAGCAATGGCGGTGGCAA
GCAGGCCCTGGAGACCGTTCAGGCGCTGTTGCCGGTGCTGTGCCAGGCGCATGGCTTAACCCCGG
AACAAGTTGTTGCGATCGCTAGCAACAACGGTGGCAAACAGGCTCTGGAGACCGTTCAGCGTCTG
TTACCGGTGCTGTGCCAGGCACATGGCCTGACCCCGGAGCAAGTTGTTGCCATTGCCAGCCACGA
TGGTGGCAAGCAGGCTCTGGAGACCGTGCAGGCCCTGCTCCCGGTGCTGTGCCAGGCCCATGGCT
TAACCCCGGAGCAAGTIGTGGCTATCGCCAGCAACGGTGGCGGTAAACAGGCGCTGGAGACCGT
TCAGCGCCTGCTACCGGTGCTGTGCCAGGCTCATGGCTTGACCCCGGAACAGGTTGTTGCCATTG
CGTCCAATATCGGCGGCAAGCAGGCGCTGGAAACCGTTCAGGCTCTGCTTCCGGTGCTGTGCCAG
GCCCATGGATTAACCCCGGAGCAGGTTGTGGCGATTGCGAGCAACGGCGGTGGCAAACAGGCCC
TGGAGACCGTGCAGGCGCTGCTACCGGTGCTGTGCCAGGCTCATGGGCTGACCCCGGAGCAAGTG
GTTGCTATTGCTAGCAATGGCGGCGGCAAGCAGGCTCTGGAAACGGTGCAGGCCCTGCTGCCGGT
GCTGTGCCAGGCCCATGGGTTAACCCCGGAACAAGTGGTGGCCATCGCTTCCAATATTGGCGGTA
AACAGGCCCTGGAGACTGTTCAGCGTCTGCTTCCGGTGCTGTGCCAGGCCCATGGGCTAACCCCG
CAGCAAGTTGTTGCTATTGCCTCCAATGGCGGTGGCAAGCAGGCTCTGGAAACTGTGCAGCGCCT
GCTGCCGGTGCTGTGCCAGGCTCACGGCCTGACCCCGCAGCAAGTTGTGGCAATCGCAAGCAATG
GTGGTGGTCGTCCGGCTCTGGAGAGCATTGTGGCGCAGCTGAGCCGTCCAGACCCGGCCCTGGCG
GCTCTGACCAATGATCACCTGGTGGCCCTGGCCTGCCTGGGTGGCCGTCCGGCTCTGGATGCCGT
GAAGAAAGGTCTGGGC
Translated amino acid sequence:
(SEQ ID NO: 151)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASNIGGKQALETVQALLPVLCQAHGLTPQQVVAIASNNGGKQALETVQRLLPVL
CQAHGLTPQQVVAIASNIGGKQALETVQRLLPVLCQAHGLTPEQVVAIASNIGGKQALETVQALLPVL
CQAHGLTPEQVVAIASHDGGKQALETVQALLPVLCQAHGLTPEQVVAIASNGGGKQALETVQALLPV
LCQAHGLTPEQVVAIASNNGGKQALETVQRLLPVLCQAHGLTPEQVVAIASHDGGKQALETVQALLP
VLCQAHGLTPEQVVAIASNGGGKQALETVQRLLPVLCQAHGLTPEQVVAIASNIGGKQALETVQALL
PVLCQAHGLTPEQVVAIASNGGGKQALETVQALLPVLCQAHGLTPEQVVAIASNGGGKQALETVQAL
LPVLCQAHGLTPEQVVAIASNIGGKQALETVQRLLPVLCQAHGLTPQQVVAIASNGGGKQALETVQR
LLPVLCQAHGLTPQQVVAIASNGGGRPALESIVAQLSRPDPALAALTNDHLVALACLGGRPALDAVK
KGLG
(J) ND2-DdCBE Left mitoTALE repeat
(SEQ ID NO: 152)
GACATCGCGGATCTGCGTACCCTGGGTTACAGCCAGCAGCAGCAGGAGAAGATCAAGCCAAAGG
TGCGCAGCACCGTGGCCCAGCACCATGAAGCTCTGGTGGGTCACGGCTTCACCCACGCGCACATC
GTGGCTCTGAGCCAGCACCCAGCCGCGCTGGGTACCGTGGCCGTGAAATATCAGGACATGATTGC
TGCCCTGCCAGAGGCTACCCATGAAGCGATTGTGGGTGTGGGCAAGCAGTGGAGCGGTGCTCGTG
CGCTGGAGGCCCTGCTGACCGTGGCCGGTGAACTGCGTGGCCCGCCGCTGCAGCTGGATACCGGC
CAGCTGCTGAAAATCGCGAAACGTGGCGGTGTGACCGCTGTGGAAGCTGTGCATGCCTGGCGTAA
TGCTCTGACCGGTGCCCCGCTGAACCTGACCCCGGAACAAGTGGTGGCTATCGCGTCCCATGATG
GTGGTAAACAGGCTCTGGAGACCGTGCAAGCTCTGCTGCCAGTGCTGTGCCAGGCCCATGGTCTG
ACCCCGCAGCAGGTGGTGGCTATTGCCAGCAATGGCGGTGGCAAGCAGGCGCTGGAGACCGTGC
AGCGTCTGCTGCCGGTGCTGTGCCAGGCTCATGGCCTGACCCCGCAGCAAGTTGTGGCTATTGCC
AGCAACGGTGGCGGTAAACAGGCCCTGGAGACCGTGCAGCGCCTGTTACCGGTGCTGTGCCAAG
CCCATGGCCTGACCCCGGAGCAGGTGGTGGCGATCGCTAGCAACATCGGCGGCAAGCAGGCTCT
GGAAACCGTGCAGGCCCTGCTTCCGGTGCTGTGCCAGGCCCATGGCTTAACCCCGGAACAGGTTG
TTGCTATTGCCAGCAACAACGGCGGTAAACAGGCGCTGGAAACCGTGCAGGCTCTGTTACCGGTG
CTGTGCCAGGCCCATGGGCTGACCCCGGAACAAGTTGTGGCTATTGCGAGCCATGATGGCGGCAA
GCAGGCCCTGGAAACCGTGCAGGCGCTGTTGCCGGTGCTGTGCCAAGCCCATGGATTAACCCCGG
AACAAGTTGTTGCGATCGCTAGCAACATTGGCGGTAAACAGGCTCTGGAAACCGTTCAGCGTCTG
TTACCGGTGCTGTGCCAGGCGCATGGTCTGACCCCGGAACAGGTTGTGGCCATTGCCTCCAATGG
CGGTGGCAAGCAGGCTCTGGAGACCGTTCAGGCCCTGCTCCCGGTGCTGTGCCAAGCGCATGGCC
TGACCCCGGAACAGGTGGTGGCTATCGCCAGCAACATTGGTGGCAAACAGGCGCTGGAGACCGT
TCAGCGCCTGCTACCGGTGCTGTGCCAGGCACATGGTCTGACCCCGGAGCAAGTTGTGGCCATTG
CTAGCCACGATGGTGGCAAGCAGGCGCTGGAAACCGTTCAGGCTCTGCTTCCGGTGCTGTGCCAG
GCCCATGGTTTAACCCCGGAACAAGTGGTTGCCATTGCGTCCAATGGTGGCGGTAAACAGGCCCT
GGAAACCGTTCAGGCGCTGCTACCGGTGCTGTGCCAGGCTCATGGGCTGACCCCGGAGCAAGTGG
TTGCTATTGCTTCCCATGATGGCGGCAAGCAGGCTCTGGAAACGGTGCAGGCCCTGCTGCCGGTG
CTGTGCCAAGCCCATGGGTTAACCCCGGAACAGGTGGTTGCGATTGCTAGCCACGACGGCGGTAA
ACAGGCCCTGGAAACGGTTCAGCGTCTGCTTCCGGTGCTGTGCCAGGCCCATGGACTTACCCCGC
AGCAGGTTGTGGCGATTGCCTCCAATGGCGGTGGCAAGCAGGCTCTGGAAACTGTGCAGCGCCTG
CTGCCGGTGCTGTGCCAGGCTCATGGTTTAACCCCGGAGCAGGTTGTTGCCATCGCCAGCCACGA
CGGTGGCAAACAGGCGCTGGAAACTGTGCAGGCTCTGCTCCCGGTGCTGTGCCAGGCTCATGGAC
TTACCCCGGAGCAGGTGGTTGCCATTGCTAGCAACATTGGTGGCAAGCAGGCCCTGGAGACTGTT
CAGGCGCTGTTACCGGTGCTGTGCCAGGCCCATGGGTTAACCCCGGAGCAAGTTGTTGCCATTGC
CTCCAATATTGGTGGCAAACAGGCTCTGGAGACTGTTCAGGCCCTGCTGCCGGTGCTGTGCCAGG
CTCACGGTCTGACCCCGCAGCAAGTGGTGGCAATCGCAAGCAATGGTGGTGGTCGTCCGGCTCTG
GAGAGCATTGTGGCGCAGCTGAGCCGTCCAGACCCGGCCCTGGCGGCTCTGACCAATGATCACCT
GGTGGCCCTGGCCTGCCTGGGTGGCCGTCCGGCTCTGGATGCCGTGAAGAAAGGTCTGGGC
Translated amino acid sequence:
(SEQ ID NO: 151)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASNIGGKQALETVQALLPVLCQAHGLTPQQVVAIASNNGGKQALETVQRLLPVL
CQAHGLTPQQVVAIASNIGGKQALETVQRLLPVLCQAHGLTPEQVVAIASNIGGKQALETVQALLPVL
CQAHGLTPEQVVAIASHDGGKQALETVQALLPVLCQAHGLTPEQVVAIASNGGGKQALETVQALLPV
LCQAHGLTPEQVVAIASNNGGKQALETVQRLLPVLCQAHGLTPEQVVAIASHDGGKQALETVQALLP
VLCQAHGLTPEQVVAIASNGGGKQALETVQRLLPVLCQAHGLTPEQVVAIASNIGGKQALETVQALL
PVLCQAHGLTPEQVVAIASNGGGKQALETVQALLPVLCQAHGLTPEQVVAIASNGGGKQALETVQAL
LPVLCQAHGLTPEQVVAIASNIGGKQALETVQRLLPVLCQAHGLTPQQVVAIASNGGGKQALETVQR
LLPVLCQAHGLTPQQVVAIASNGGGRPALESIVAQLSRPDPALAALTNDHLVALACLGGRPALDAVK
KGLG
(K) ND4-DdCBE Right mitoTALE repeat
(SEQ ID NO: 153)
GACATCGCGGATCTGCGTACCCTGGGTTACAGCCAGCAGCAGCAGGAGAAGATCAAGCCAAAGG
TGCGCAGCACCGTGGCCCAGCACCATGAAGCTCTGGTGGGTCACGGCTTCACCCACGCGCACATC
GTGGCTCTGAGCCAGCACCCAGCCGCGCTGGGTACCGTGGCCGTGAAATATCAGGACATGATTGC
TGCCCTGCCAGAGGCTACCCATGAAGCGATTGTGGGTGTGGGCAAGCAGTGGAGCGGTGCTCGTG
CGCTGGAGGCCCTGCTGACCGTGGCCGGTGAACTGCGTGGCCCGCCGCTGCAGCTGGATACCGGC
CAGCTGCTGAAAATCGCGAAACGTGGCGGTGTGACCGCTGTGGAAGCTGTGCATGCCTGGCGTAA
TGCTCTGACCGGTGCCCCGCTGAACCTGACCCCGGAACAAGTGGTTGCGATTGCGTCCCATGATG
GTGGTAAACAAGCCCTGGAGACCGTTCAAGCTCTGCTGCCAGTGCTGTGCCAGGCTCATGGTCTG
ACCCCGCAGCAGGTGGTGGCTATTGCCAGCCATGATGGCGGCAAGCAGGCTCTGGAGACCGTGC
AGCGTCTGCTGCCGGTGCTGTGCCAAGCCCATGGCCTGACCCCGCAGCAAGTTGTGGCTATTGCC
AGCAACGGCGGTGGCAAACAGGCGCTGGAAACCGTGCAGCGCCTGTTACCGGTGCTGTGCCAAG
CGCATGGTCTGACCCCGGAGCAGGTGGTGGCCATCGCTAGCAACAACGGTGGCAAGCAGGCCCT
GGAAACCGTGCAGGCGCTGTTGCCGGTGCTGTGCCAGGCCCATGGCTTAACCCCGGAACAAGTGG
TGGCCATTGCGAGCAATGGTGGCGGTAAACAGGCTCTGGAAACCGTGCAGGCCCTGCTTCCGGTG
CTGTGCCAGGCCCATGGGCTGACCCCGGAACAAGTTGTGGCTATCGCCAGCAACATCGGCGGCAA
GCAGGCGCTGGAGACCGTTCAGGCTCTGTTACCGGTGCTGTGCCAGGCGCATGGCCTGACCCCGG
AACAGGTGGTGGCGATCGCTAGCAACATTGGCGGTAAACAGGCCCTGGAGACCGTTCAGCGCCT
GCTCCCGGTGCTGTGCCAGGCCCATGGTCTGACCCCGGAACAGGTTGTTGCTATTGCCAGCAACA
ACGGCGGCAAGCAGGCCCTGGAGACCGTGCAGGCGCTGCTACCGGTGCTGTGCCAGGCCCATGG
ACTGACCCCGGAGCAGGTTGTGGCCATCGCGTCCAATGGCGGTGGCAAACAGGCTCTGGAGACC
GTTCAGCGTCTGTTACCGGTGCTGTGCCAGGCACATGGCCTGACCCCGGAGCAAGTTGTTGCCAT
CGCTAGCAACATTGGTGGCAAGCAGGCGCTGGAAACCGTTCAGCGCCTGCTACCGGTGCTGTGCC
AGGCTCATGGCTTAACCCCGGAGCAGGTTGTCGCCATTGCCAGCAACAATGGTGGCAAACAGGCT
CTGGAAACTGTGCAGGCCCTGCTACCGGTGCTGTGCCAGGCCCATGGGTTAACCCCGGAACAGGT
TGTGGCCATTGCCTCCAATAACGGTGGCAAGCAGGCGCTGGAAACGGTGCAGGCTCTGCTTCCGG
TGCTGTGCCAGGCTCATGGGCTGACCCCGGAGCAAGTGGTTGCTATTGCGTCCAACATTGGTGGC
AAACAGGCCCTGGAAACCGTTCAGGCGCTGCTCCCGGTGCTGTGCCAGGCCCATGGGCTAACCCC
GGAACAGGTGGTTGCCATTGCCTCCAACAATGGTGGCAAGCAGGCCCTGGAAACGGTTCAGCGTC
TGCTTCCGGTGCTGTGCCAGGCCCATGGGCTTACCCCGCAGCAAGTTGTTGCTATCGCCAGCAAT
ATTGGTGGCAAACAGGCTCTGGAAACGGTGCAGCGCCTGCTACCGGTGCTGTGCCAGGCTCATGG
TTTAACCCCGCAGCAGGTGGTTGCGATTGCCTCCAACAACGGTGGCAAGCAGGCGCTGGAAACTG
TTCAGCGTCTGCTCCCGGTGCTGTGCCAGGCTCACGGCCTGACCCCGCAGCAAGTGGTGGCTATC
GCCTCCAACGGTGGTGGTCGCCCGGCTCTGGAAAGCATTGTGGCGCAGCTGAGCCGTCCAGACCC
GGCCCTGGCGGCTCTGACCAATGATCACCTGGTGGCCCTGGCCTGCCTGGGTGGCCGTCCGGCTC
TGGATGCCGTGAAGAAAGGTCTGGGC
Translated amino acid sequence:
(SEQ ID NO: 154)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASHDGGKQALETVQALLPVLCQAHGLTPQQVVAIASHDGGKQALETVQRLLPV
LCQAHGLTPQQVVAIASNGGGKQALETVQRLLPVLCQAHGLTPEQVVAIASNNGGKQALETVQALLP
VLCQAHGLTPEQVVAIASNGGGKQALETVQALLPVLCQAHGLTPEQVVAIASNIGGKQALETVQALL
PVLCQAHGLTPEQVVAIASNIGGKQALETVQRLLPVLCQAHGLTPEQVVAIASNNGGKQALETVQAL
LPVLCQAHGLTPEQVVAIASNGGGKQALETVQRLLPVLCQAHGLTPEQVVAIASNIGGKQALETVQR
LLPVLCQAHGLTPEQVVAIASNNGGKQALETVQALLPVLCQAHGLTPEQVVAIASNNGGKQALETVQ
ALLPVLCQAHGLTPEQVVAIASNIGGKQALETVQALLPVLCQAHGLTPEQVVAIASNNGGKQALETV
QRLLPVLCQAHGLTPQQVVAIASNIGGKQALETVQRLLPVLCQAHGLTPQQVVAIASNNGGKQALET
VQRLLPVLCQAHGLTPQQVVAIASNGGGRPALESIVAQLSRPDPALAALTNDHLVALACLGGRPALD
AVKKGLG
(L) ND4-DdCBE Left mitoTALE repeat
(SEQ ID NO: 155)
GACATCGCGGATCTGCGTACCCTGGGTTACAGCCAGCAGCAGCAGGAGAAGATCAAGCCAAAGG
TGCGCAGCACCGTGGCCCAGCACCATGAAGCTCTGGTGGGTCACGGCTTCACCCACGCGCACATC
GTGGCTCTGAGCCAGCACCCAGCCGCGCTGGGTACCGTGGCCGTGAAATATCAGGACATGATTGC
TGCCCTGCCAGAGGCTACCCATGAAGCGATTGTGGGTGTGGGCAAGCAGTGGAGCGGTGCTCGTG
CGCTGGAGGCCCTGCTGACCGTGGCCGGTGAACTGCGTGGCCCGCCGCTGCAGCTGGATACCGGC
CAGCTGCTGAAAATCGCGAAACGTGGCGGTGTGACCGCTGTGGAAGCTGTGCATGCCTGGCGTAA
TGCTCTGACCGGTGCCCCGCTGAACCTGACCCCGGAACAGGTGGTGGCAATCGCAAGCAATAATG
GTGGTAAACAGGCTCTGGAAACCGTGCAAGCTCTGCTGCCAGTTCTGTGCCAGGCTCATGGTCTG
ACCCCGCAGCAGGTGGTGGCTATTGCCAGCCATGATGGCGGCAAGCAGGCCCTGGAGACCGTGC
AGCGTCTGCTGCCGGTGCTGTGCCAGGCCCATGGCCTGACCCCGCAGCAAGTTGTGGCTATTGCC
AGCAACGGCGGTGGCAAACAGGCTCTGGAGACCGTGCAGCGCCTGTTACCGGTGCTGTGCCAAG
CCCATGGTCTGACCCCGGAGCAGGTGGTGGCGATCGCTAGCAACATTGGTGGCAAGCAGGCCCTG
GAAACCGTGCAGGCGCTGTTGCCGGTGCTGTGCCAAGCCCATGGGCTGACCCCGGAACAAGTTGT
TGCCATTGCCAGCAACAATGGTGGCAAACAGGCTCTGGAAACTGTGCAGGCCCTGCTTCCGGTGC
TGTGCCAGGCCCATGGATTAACCCCGGAACAAGTTGTGGCTATTGCGAGCAATGGCGGCGGCAA
GCAGGCGCTGGAAACCGTGCAGGCTCTGTTACCGGTGCTGTGCCAGGCGCATGGCCTGACCCCGG
AGCAAGTGGTGGCCATCGCTAGCAACATTGGCGGTAAACAGGCGCTGGAGACCGTTCAGCGTCT
GTTACCGGTGCTGTGCCAGGCACATGGCCTTACCCCGGAACAAGTTGTGGCCATTGCCAGCAACA
TCGGCGGCAAGCAGGCCCTGGAAACGGTGCAGGCGCTGCTCCCGGTGCTGTGCCAGGCCCATGG
GTTAACCCCGGAACAAGTGGTTGCTATTGCTAGCCATGATGGCGGTAAACAGGCCCTGGAGACCG
TTCAGCGCCTGCTACCGGTGCTGTGCCAGGCCCATGGTTTAACCCCGGAACAGGTTGTTGCGATT
GCTAGCCACGATGGCGGCAAGCAGGCTCTGGAGACCGTTCAGGCCCTGCTCCCGGTGCTGTGCCA
GGCCCATGGGCTTACCCCGGAGCAAGTTGTTGCTATTGCCTCCAATATTGGCGGTAAACAGGCGC
TGGAAACCGTTCAGGCTCTGCTTCCGGTGCTGTGCCAGGCTCATGGCCTCACCCCGGAACAAGTT
GTGGCGATTGCGTCCCATGATGGCGGCAAGCAGGCCCTGGAAACTGTGCAGGCGCTGCTACCGGT
GCTGTGCCAGGCCCATGGGCTAACCCCGGAACAGGTGGTTGCGATTGCTAGCAACAACGGCGGT
AAACAGGCTCTGGAGACTGTTCAGCGTCTGCTTCCGGTGCTGTGCCAGGCTCATGGGCTGACCCC
GCAGCAAGTGGTTGCTATTGCCAGCAATGGCGGTGGCAAGCAGGCGCTGGAGACTGTTCAGCGC
CTGCTCCCGGTGCTGTGCCAGGCTCATGGTTTAACCCCGGAGCAGGTTGTGGCGATCGCCAGCAA
TGGTGGCGGTAAACAGGCTCTGGAAACGGTGCAGGCCCTGCTCCCGGTGCTGTGCCAGGCTCATG
GACTGACCCCGGAGCAAGTTGTTGCCATTGCGTCCCACGACGGCGGCAAGCAGGCGCTGGAGAC
GGTGCAGGCTCTGCTCCCGGTGCTGTGCCAGGCTCACGGTCTGACCCCGCAACAGGTGGTGGCAA
TCGCAAGCAACGGTGGTGGTCGTCCGGCACTGGAGAGCATTGTGGCGCAGCTGAGCCGTCCAGA
CCCGGCCCTGGCGGCTCTGACCAATGATCACCTGGTGGCCCTGGCCTGCCTGGGTGGCCGTCCGG
CTCTGGATGCCGTGAAGAAAGGTCTGGGC
Translated amino acid sequence:
(SEQ ID NO: 156)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASNNGGKQALETVQALLPVLCQAHGLTPQQVVAIASHDGGKQALETVQRLLPV
LCQAHGLTPQQVVAIASNGGGKQALETVQRLLPVLCQAHGLTPEQVVAIASNIGGKQALETVQALLP
VLCQAHGLTPEQVVAIASNNGGKQALETVQALLPVLCQAHGLTPEQVVAIASNGGGKQALETVQALL
PVLCQAHGLTPEQVVAIASNIGGKQALETVQRLLPVLCQAHGLTPEQVVAIASNIGGKQALETVQALL
PVLCQAHGLTPEQVVAIASHDGGKQALETVQRLLPVLCQAHGLTPEQVVAIASHDGGKQALETVQAL
LPVLCQAHGLTPEQVVAIASNIGGKQALETVQALLPVLCQAHGLTPEQVVAIASHDGGKQALETVQA
LLPVLCQAHGLTPEQVVAIASNNGGKQALETVQRLLPVLCQAHGLTPQQVVAIASNGGGKQALETVQ
RLLPVLCQAHGLTPEQVVAIASNGGGKQALETVQALLPVLCQAHGLTPEQVVAIASHDGGKQALETV
QALLPVLCQAHGLTPQQVVAIASNGGGRPALESIVAQLSRPDPALAALTNDHLVALACLGGRPALDA
VKKGLG
(M) ND5.1-DdCBE Right mitoTALE repeat
(SEQ ID NO: 157)
GACATCGCGGATCTGCGTACCCTGGGTTACAGCCAGCAGCAGCAGGAGAAGATCAAGCCAAAGG
TGCGCAGCACCGTGGCCCAGCACCATGAAGCTCTGGTGGGTCACGGCTTCACCCACGCGCACATC
GTGGCTCTGAGCCAGCACCCAGCCGCGCTGGGTACCGTGGCCGTGAAATATCAGGACATGATTGC
TGCCCTGCCAGAGGCTACCCATGAAGCGATTGTGGGTGTGGGCAAGCAGTGGAGCGGTGCTCGTG
CGCTGGAGGCCCTGCTGACCGTGGCCGGTGAACTGCGTGGCCCGCCGCTGCAGCTGGATACCGGC
CAGCTGCTGAAAATCGCGAAACGTGGCGGTGTGACCGCTGTGGAAGCTGTGCATGCCTGGCGTAA
TGCTCTGACCGGTGCCCCGCTGAACCTGACCCCACAGCAGGTGGTGGCAATCGCAAGCCACGACG
GAGGCAAGCAGGCCCTGGAGACCGTGCAGAGGCTGCTGCCCGTGCTGTGCCAGGCACACGGACT
GACACCTGAACAGGTCGTGGCAATCGCATCCAACGGAGGCGGCAAGCAGGCCCTGGAAACCGTG
CAGCGCCTGTTACCCGTGCTGTGCCAGGCCCACGGCCTGACACCCCAGCAGGTGGTGGCCATCGC
CTCTAATGGAGGGGGCAAGCAGGCCCTGGAGACGGTGCAGCGGCTGCTGCCTGTGCTGTGCCAG
GCTCATGGACTGACACCAGAACAGGTGGTCGCAATCGCAAGCAACGGAGGTGGCAAGCAGGCCC
TGGAGACTGTGCAGGCCCTGCTTCCCGTGCTGTGCCAGGCTCACGGACTGACACCTCAGCAGGTC
GTCGCCATCGCCTCCAACAATGGTGGCAAGCAGGCCCTGGAGACAGTGCAGAGACTGCTGCCAG
TGCTGTGCCAAGCCCATGGACTGACACCACAGCAGGTCGTCGCTATCGCCTCTAATAACGGCGGC
AAGCAGGCCCTGGAGACGGTACAGAGGCTGTTACCCGTGCTGTGCCAAGCACACGGACTGACAC
CAGAGCAGGTCGTCGCAATCGCCAGCAATATCGGTGGCAAGCAGGCCCTGGAGACGGTCCAGCG
CCTGCTCCCCGTGCTGTGCCAAGCCCACGGCCTGACCCCTCAGCAGGTCGTGGCTATTGCTAGCA
ATAACGGGGGCAAGCAGGCCCTGGAGACGGTTCAGCGGCTGTTGCCCGTGCTGTGCCAAGCCCA
CGGTCTGACCCCTCAGCAGGTGGTCGCTATTGCTTCTAATGGAGGAGGCAAGCAGGCCCTGGAGA
CGGTACAGAGACTGTTACCTGTGCTGTGCCAGGCACATGGCCTGACACCAGAGCAGGTGGTCGCT
ATCGCCAGCAACATAGGTGGCAAGCAGGCCCTGGAGACGGTACAGAGGCTGCTTCCCGTGCTGT
GCCAAGCTCATGGCCTGACACCTGAACAGGTGGTCGCCATTGCTAGCAATAACGGTGGCAAGCA
GGCCCTGGAGACGGTACAGCGGCTGTTACCAGTGCTGTGCCAAGCACATGGCTTAACCCCTCAAC
AGGTCGTCGCAATTGCCTCTAATATCGGAGGCAAGCAGGCCCTGGAGACGGTACAGCGGCTGCTC
CCCGTGCTGTGCCAGGCGCACGGCCTGACTCCTCAGCAGGTCGTGGCAATCGCCAGCAACATCGG
CGGCAGACCTGCCCTGGAGAGCATTGTGGCGCAGCTGAGCCGTCCAGACCCGGCCCTGGCGGCTC
TGACCAATGATCACCTGGTGGCCCTGGCCTGCCTGGGTGGCCGTCCGGCTCTGGATGCCGTGAAG
AAAGGTCTGGGC
Translated amino acid sequence:
(SEQ ID NO: 11)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPQQVVAIASHDGGKQALETVQRLLPVLCQAHGLTPEQVVAIASNGGGKQALETVQRLLPV
LCQAHGLTPQQVVAIASNGGGKQALETVQRLLPVLCQAHGLTPEQVVAIASNGGGKQALETVQALLP
VLCQAHGLTPQQVVAIASNNGGKQALETVQRLLPVLCQAHGLTPQQVVAIASNNGGKQALETVQRL
LPVLCQAHGLTPEQVVAIASNIGGKQALETVQRLLPVLCQAHGLTPQQVVAIASNNGGKQALETVQR
LLPVLCQAHGLTPQQVVAIASNGGGKQALETVQRLLPVLCQAHGLTPEQVVAIASNIGGKQALETVQ
RLLPVLCQAHGLTPEQVVAIASNNGGKQALETVQRLLPVLCQAHGLTPQQVVAIASNIGGKQALETV
QRLLPVLCQAHGLTPQQVVAIASNIGGRPALESIVAQLSRPDPALAALTNDHLVALACLGGRPALDAV
KKGLG
(N) ND5.1-DdCBE Left mitoTALE repeat
(SEQ ID NO: 158)
CTGACCCCTGAGCAGGTGGTGGCCATCGCCAGCAATATCGGAGGCAAGCAGGCCCTGGAGACCG
TGCAGGCCCTGCTGCCCGTGCTGTGCCAGGCACACGGACTGACACCTCAGCAGGTCGTCGCCATC
GCCTCCAACAATGGCGGCAAGCAGGCCCTGGAAACCGTGCAGAGGCTGTTACCCGTGCTGTGCCA
GGCCCACGGCCTGACACCCCAGCAGGTGGTGGCAATCGCATCTCACGATGGGGGCAAGCAGGCC
CTGGAGACGGTGCAGCGCCTGCTGCCTGTGCTGTGCCAGGCTCATGGACTGACACCAGAACAGGT
CGTGGCCATCGCCAGCAACATTGGCGGCAAGCAGGCCCTGGAGACTGTCCAGGCCCTGTTACCCG
TGCTGTGCCAAGCCCATGGACTGACACCTGAACAGGTCGTGGCAATCGCATCCAATGGAGGTGGC
AAGCAGGCCCTGGAGACAGTGCAGGCCCTGCTGCCAGTGCTGTGCCAGGCTCACGGCCTGACACC
AGAACAGGTGGTCGCAATCGCATCTAATGGAGGAGGCAAGCAGGCCCTGGAGACGGTACAGGCC
CTGTTGCCCGTGCTGTGCCAAGCCCACGGACTGACACCAGAGCAGGTCGTCGCTATTGCTTCCAA
CATTGGAGGCAAGCAGGCCCTGGAGACGGTCCAGCGGCTGCTTCCCGTGCTGTGCCAAGCTCATG
GCCTGACACCAGAGCAGGTGGTCGCTATTGCCTCCAACAATGGAGGCAAGCAGGCCCTGGAGAC
GGTTCAGGCCCTGCTTCCCGTGCTGTGCCAGGCTCATGGTCTGACACCCGAACAGGTGGTCGCTA
TCGCCTCTCACGATGGAGGCAAGCAGGCCCTGGAGACGGTACAGAGGCTGTTACCTGTGCTGTGC
CAGGCCCATGGGCTGACCCCAGAACAGGTGGTCGCCATCGCCAGCAACATCGGCGGCAAGCAGG
CCCTGGAGACGGTACAGGCCCTGCTCCCCGTGCTGTGCCAAGCACATGGCCTGACACCCGAGCAG
GTCGTGGCTATTGCTAGCAACAACGGGGGCAAGCAGGCCCTGGAGACGGTACAGGCCCTGCTAC
CAGTGCTGTGCCAAGCGCACGGGCTGACCCCAGAGCAGGTCGTCGCAATCGCCTCTAACAACGGT
GGCAAGCAGGCCCTGGAGACGGTACAGGCCCTGCTGCCCGTGCTGTGCCAAGCGCATGGGCTGA
CTCCAGAACAGGTGGTGGCTATCGCCAGCAACATTGGAGGCAAGCAGGCCCTGGAGACGGTACA
GCGGCTGCTACCCGTGCTGTGCCAAGCGCACGGTCTGACACCTCAGCAGGTGGTCGCTATCGCTT
CTAACATAGGGGGCAAGCAGGCCCTGGAGACGGTACAGCGGCTGCTGCCCGTGCTGTGCCAAGC
GCACGGACTGACCCCACAGCAGGTCGTCGCTATCGCCTCTAACGGAGGAGGCAGACCCGCCCTG
GAG
Translated amino acid sequence:
(SEQ ID NO: 159)
LTPEQVVAIASNIGGKQALETVQALLPVLCQAHGLTPQQVVAIASNNGGKQALETVQRLLPVLCQAH
GLTPQQVVAIASHDGGKQALETVQRLLPVLCQAHGLTPEQVVAIASNIGGKQALETVQALLPVLCQA
HGLTPEQVVAIASNGGGKQALETVQALLPVLCQAHGLTPEQVVAIASNGGGKQALETVQALLPVLCQ
AHGLTPEQVVAIASNIGGKQALETVQRLLPVLCQAHGLTPEQVVAIASNNGGKQALETVQALLPVLC
QAHGLTPEQVVAIASHDGGKQALETVQRLLPVLCQAHGLTPEQVVAIASNIGGKQALETVQALLPVL
CQAHGLTPEQVVAIASNNGGKQALETVQALLPVLCQAHGLTPEQVVAIASNNGGKQALETVQALLPV
LCQAHGLTPEQVVAIASNIGGKQALETVQRLLPVLCQAHGLTPQQVVAIASNIGGKQALETVQRLLPV
LCQAHGLTPQQVVAIASNGGGRPALE
(O) ND5.2-DdCBE Right mitoTALE repeat
(SEQ ID NO: 160)
GACATCGCGGATCTGCGTACCCTGGGTTACAGCCAGCAGCAGCAGGAGAAGATCAAGCCGAAGG
TGCGCAGCACCGTGGCTCAGCACCACGAAGCCCTGGTGGGCCACGGTTTCACCCACGCTCACATC
GTGGCCCTGAGCCAGCACCCAGCCGCGCTGGGCACCGTGGCCGTGAAATATCAGGACATGATTGC
TGCCCTGCCAGAGGCCACCCATGAAGCTATTGTGGGCGTGGGCAAGCAGTGGAGCGGTGCTCGTG
CGCTGGAGGCGCTGCTGACCGTGGCTGGTGAACTGCGTGGTCCGCCGCTGCAGCTGGATACCGGT
CAGCTGCTGAAAATTGCCAAACGTGGCGGTGTGACCGCGGTGGAAGCCGTGCATGCTTGGCGTAA
TGCTCTGACCGGTGCGCCGCTGAACCTGACCCCGCAGCAGGTGGTGGCTATTGCCAGCAACAACG
GCGGTAAACAGGCTCTGGAGACCGTGCAGCGTCTGCTGCCGGTGCTGTGCCAGGCTCATGGTCTG
ACCCCGGAGCAGGTGGTGGCCATTGCTAGCCATGATGGCGGCAAGCAGGCGCTGGAAACCGTGC
AGCGCCTGTTACCGGTGCTGTGCCAAGCCCATGGTCTGACCCCGCAGCAAGTTGTGGCTATTGCG
AGCAACGGCGGTGGCAAACAGGCCCTGGAAACCGTTCAGCGTCTGTTACCGGTGCTGTGCCAGGC
CCATGGCCTGACCCCGGAACAAGTGGTGGCTATCGCCAGCAACATTGGTGGCAAGCAGGCCCTG
GAAACCGTGCAGGCGCTGTTGCCGGTGCTGTGCCAAGCCCATGGGCTGACCCCGCAGCAAGTGGT
TGCGATCGCTAGCAACAACGGTGGCAAACAGGCTCTGGAAACCGTTCAGCGCCTGCTTCCGGTGC
TGTGCCAAGCGCATGGCTTAACCCCGCAGCAAGTTGTGGCCATTGCGAGCAACAACGGTGGCAA
GCAGGCGCTGGAGACCGTTCAGCGTCTGCTTCCGGTGCTGTGCCAGGCGCATGGCCTGACCCCGG
AGCAAGTGGTGGCTATTGCTAGCCACGATGGTGGCAAACAGGCCCTGGAGACCGTGCAGCGCCT
GCTCCCGGTGCTGTGCCAGGCCCATGGATTAACCCCGCAGCAAGTGGTGGCCATCGCCAGCAATG
GCGGCGGCAAGCAGGCTCTGGAAACTGTGCAGCGTCTGTTACCGGTGCTGTGCCAGGCCCATGGG
TTAACCCCGCAGCAGGTTGTTGCCATTGCCTCCAATAATGGCGGTAAACAGGCGCTGGAGACTGT
GCAGCGCCTGCTACCGGTGCTGTGCCAGGCACATGGTCTGACCCCGGAACAAGTTGTTGCCATTG
CGTCCCATGATGGCGGCAAGCAGGCCCTGGAGACTGTTCAGCGTCTGCTCCCGGTGCTGTGCCAG
GCCCATGGTTTAACCCCGGAACAAGTTGTGGCCATTGCTAGCCACGATGGCGGTAAACAGGCTCT
GGAAACTGTTCAGCGCCTGCTGCCGGTGCTGTGCCAAGCACATGGCTTAACCCCGGAACAGGTTG
TTGCTATTGCCAGCAACATCGGCGGCAAGCAGGCTCTGGAGACCGTTCAGGCCCTGTTGCCGGTG
CTGTGCCAGGCCCATGGGCTTACCCCGGAACAAGTGGTTGCCATCGCCAGCAACATTGGCGGTAA
ACAGGCGCTGGAAACCGTTCAGGCTCTGTTGCCGGTGCTGTGCCAGGCTCATGGCCTTACCCCGC
AGCAAGTTGTGGCGATTGCTAGCAATGGCGGTGGCAAGCAGGCGCTGGAGACGGTTCAGCGTCT
GCTACCGGTGCTGTGCCAGGCTCATGGATTGACCCCGCAGCAGGTCGTGGCCATTGCCTCCAATA
ACGGTGGCAAACAGGCGCTGGAGACAGTTCAGCGCCTGCTGCCGGTGCTGTGCCAGGCTCATGG
GTTGACCCCGCAGCAGGTAGTTGCTATTGCTAGCAATGGTGGCGGTCGTCCGGCCCTGGAGAGCA
TTGTGGCGCAGCTGAGCCGTCCAGACCCGGCGCTGGCGGCTCTGACCAACGATCACCTGGTGGCG
CTGGCTTGCCTGGGCGGTCGTCCGGCCCTGGATGCCGTGAAGAAAGGCCTGGGT
Translated amino acid sequence:
(SEQ ID NO: 161)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPQQVVAIASNNGGKQALETVQRLLPVLCQAHGLTPEQVVAIASHDGGKQALETVQRLLPV
LCQAHGLTPQQVVAIASNGGGKQALETVQRLLPVLCQAHGLTPEQVVAIASNIGGKQALETVQALLP
VLCQAHGLTPQQVVAIASNNGGKQALETVQRLLPVLCQAHGLTPQQVVAIASNNGGKQALETVQRL
LPVLCQAHGLTPEQVVAIASHDGGKQALETVQRLLPVLCQAHGLTPQQVVAIASNGGGKQALETVQR
LLPVLCQAHGLTPQQVVAIASNNGGKQALETVQRLLPVLCQAHGLTPEQVVAIASHDGGKQALETVQ
RLLPVLCQAHGLTPEQVVAIASHDGGKQALETVQRLLPVLCQAHGLTPEQVVAIASNIGGKQALETV
QALLPVLCQAHGLTPEQVVAIASNIGGKQALETVQALLPVLCQAHGLTPQQVVAIASNGGGKQALET
VQRLLPVLCQAHGLTPQQVVAIASNNGGKQALETVQRLLPVLCQAHGLTPQQVVAIASNGGGRPALE
SIVAQLSRPDPALAALTNDHLVALACLGGRPALDAVKKGLG
(P) ND5.2-DdCBE Left mitoTALE repeat
(SEQ ID NO: 162)
GACATCGCGGATCTGCGTACCCTGGGTTACAGCCAGCAGCAGCAGGAGAAGATCAAGCCGAAGG
TGCGCAGCACCGTGGCTCAGCACCACGAAGCCCTGGTGGGCCACGGTTTCACCCACGCTCACATT
GTGGCCCTGAGCCAGCACCCAGCCGCGCTGGGCACCGTGGCCGTGAAATATCAGGATATGATTGC
TGCCCTGCCAGAGGCCACCCATGAAGCTATTGTGGGCGTGGGCAAGCAGTGGAGCGGTGCTCGTG
CGCTGGAGGCGCTGCTGACCGTGGCTGGTGAACTGCGTGGTCCGCCGCTGCAGCTGGACACCGGT
CAGCTGCTGAAAATCGCGAAACGTGGCGGTGTGACCGCGGTGGAAGCCGTGCATGCTTGGCGTA
ATGCTCTGACCGGTGCGCCGCTGAACCTGACCCCTGAGCAGGTGGTGGCAATCGCAAGCCACGAC
GGAGGCAAGCAGGCCCTGGAGACAGTGCAGGCCCTGCTGCCCGTGCTGTGCCAGGCACACGGCC
TGACACCTGAGCAGGTGGTGGCCATCGCCTCCAACATCGGCGGCAAGCAGGCCCTGGAGACAGT
ACAGAGGCTGTTACCCGTGCTGTGCCAGGCCCACGGCCTGACACCCCAGCAGGTCGTCGCCATCG
CCTCTAATATTGGAGGCAAGCAGGCCCTGGAGACAGTCCAGCGCCTGCTGCCTGTGCTGTGCCAG
GCTCATGGCCTGACACCAGAACAGGTCGTGGCCATCGCCAGTAATATTGGGGGCAAGCAGGCCCT
GGAGACAGTTCAGGCCCTGTTACCCGTGCTGTGCCAAGCCCATGGCCTGACACCTGAACAGGTGG
TCGCCATCGCCTCCAATATTGGTGGCAAGCAGGCCCTGGAGACAGTACAGGCCCTGCTGCCAGTG
CTGTGCCAGGCTCACGGCCTGACACCAGAGCAGGTCGTCGCAATCGCATCTCATGATGGCGGCAA
GCAGGCCCTGGAGACAGTACAGGCCCTGTTACCCGTGCTGTGCCAAGCGCACGGCCTGACCCCTG
AACAGGTCGTGGCTATTGCAAGCCACGATGGTGGCAAGCAGGCCCTGGAGACAGTACAGCGGCT
GCTTCCCGTGCTGTGCCAAGCTCATGGCCTGACACCTGAGCAGGTCGTCGCTATTGCTAGCAATAT
TGGCGGCAAGCAGGCCCTGGAGACAGTACAGGCCCTGCTCCCCGTGCTGTGCCAAGCACACGGC
CTGACACCCGAACAGGTGGTGGCTATCGCCTCTAATGGAGGTGGCAAGCAGGCCCTGGAGACAG
TACAGAGGCTGCTTCCTGTGCTGTGCCAGGCCCATGGCCTGACCCCTGAGCAGGTCGTGGCTATT
GCCAGTAATATAGGAGGCAAGCAGGCCCTGGAGACAGTACAGGCCCTGCTACCCGTGCTGTGCC
AAGCGCATGGCCTGACCCCAGAACAGGTCGTGGCAATCGCATCTCATGACGGCGGCAAGCAGGC
CCTGGAGACAGTACAGGCCCTGCTACCAGTGCTGTGCCAAGCACATGGCCTGACCCCCGAACAGG
TGGTGGCAATCGCCTCTCACGACGGGGGCAAGCAGGCCCTGGAGACAGTACAGGCCCTGCTACC
CGTGCTGTGCCAAGCGCACGGCCTGACGCCAGAACAGGTGGTCGCTATCGCAAGCAACGGCGGT
GGCAAGCAGGCCCTGGAGACAGTACAGCGGCTGCTACCCGTGCTGTGCCAAGCGCACGGCCTGA
CTCCTCAGCAGGTCGTCGCTATCGCATCTCATGATGGTGGCAAGCAGGCCCTGGAGACAGTACAG
CGGCTGCTACCCGTGCTGTGCCAAGCGCACGGCCTGACACCACAGCAGGTCGTCGCAATTGCATC
TAACGGAGGAGGCAGACCCGCCCTGGAGAGCATTGTGGCTCAGCTGAGCCGTCCAGACCCGGCC
CTGGCGGCTCTGACCAACGATCACCTGGTGGCGCTGGCTTGCCTGGGCGGTCGTCCGGCCCTGGA
TGCGGTGAAGAAAGGCCTGGGT
Translated amino acid sequence:
(SEQ ID NO: 163)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASHDGGKQALETVQALLPVLCQAHGLTPEQVVAIASNIGGKQALETVQRLLPVL
CQAHGLTPQQVVAIASNIGGKQALETVQRLLPVLCQAHGLTPEQVVAIASNIGGKQALETVQALLPVL
CQAHGLTPEQVVAIASNIGGKQALETVQALLPVLCQAHGLTPEQVVAIASHDGGKQALETVQALLPV
LCQAHGLTPEQVVAIASHDGGKQALETVQRLLPVLCQAHGLTPEQVVAIASNIGGKQALETVQALLP
VLCQAHGLTPEQVVAIASNGGGKQALETVQRLLPVLCQAHGLTPEQVVAIASNIGGKQALETVQALL
PVLCQAHGLTPEQVVAIASHDGGKQALETVQALLPVLCQAHGLTPEQVVAIASHDGGKQALETVQAL
LPVLCQAHGLTPEQVVAIASNGGGKQALETVQRLLPVLCQAHGLTPQQVVAIASHDGGKQALETVQR
LLPVLCQAHGLTPQQVVAIASNGGGRPALESIVAQLSRPDPALAALTNDHLVALACLGGRPALDAVK
KGLG
(Q) ND5.3-DdCBE Right mitoTALE repeat
(SEQ ID NO: 164)
GACATCGCGGATCTGCGTACCCTGGGTTACAGCCAGCAGCAGCAGGAGAAGATCAAGCCAAAGG
TGCGCAGCACCGTGGCCCAGCACCATGAAGCTCTGGTGGGTCACGGCTTCACCCACGCGCACATC
GTGGCTCTGAGCCAGCACCCAGCCGCGCTGGGTACCGTGGCCGTGAAATATCAGGACATGATTGC
TGCCCTGCCAGAGGCTACCCATGAAGCGATTGTGGGTGTGGGCAAGCAGTGGAGCGGTGCTCGTG
CGCTGGAGGCCCTGCTGACCGTGGCCGGTGAACTGCGTGGCCCGCCGCTGCAGCTGGATACCGGC
CAGCTGCTGAAAATCGCGAAACGTGGCGGTGTGACCGCTGTGGAAGCTGTGCATGCCTGGCGTAA
TGCTCTGACCGGTGCCCCGCTGAACCTGACACCACAGCAGGTCGTCGCTATCGCTTCAAACATTG
GGGGGAAACAGGCACTGGAAACCGTCCAGAGACTGCTGCCCGTCCTGTGCCAGGCCCACGGCCT
GACCCCTGAGCAGGTGGTGGCCATCGCCAGCAATATCGGAGGCAAGCAGGCCCTGGAGACCGTG
CAGCGGCTGCTGCCCGTGCTGTGCCAAGCCCACGGCTTAACACCTCAGCAGGTCGTGGCTATCGC
CTCCAACAATGGCGGCAAGCAGGCCCTGGAGACGGTGCAGAGACTGCTGCCAGTGCTGTGCCAG
GCCCACGGCTTAACACCAGAACAGGTCGTGGCCATCGCCTCTAACATTGGCGGCAAGCAGGCCCT
GGAGACTGTGCAGGCCCTGCTGCCCGTGCTGTGCCAGGCCCACGGCCTTACACCACAGCAGGTGG
TGGCAATCGCCAGCAATGGAGGGGGCAAGCAGGCCCTGGAGACAGTGCAGAGGCTGCTGCCCGT
GCTGTGCCAAGCCCACGGCCTGACACCTCAGCAGGTGGTCGCCATCGCCTCCAACGGAGGTGGCA
AGCAGGCCCTGGAGACGGTACAGCGCCTGCTGCCCGTGCTGTGCCAAGCCCACGGCCTAACACCC
GAACAGGTCGTCGCCATCGCCTCTAACATCGGCGGCAAGCAGGCCCTGGAGACGGTCCAGCGGC
TGCTGCCTGTGCTGTGCCAAGCCCACGGCCTTACCCCTCAGCAGGTCGTGGCAATCGCCAGCAAC
AATGGTGGCAAGCAGGCCCTGGAGACGGTTCAGAGACTGCTGCCCGTGCTGTGCCAAGCCCACG
GCCTCACACCTCAGCAGGTGGTGGCCATTGCCTCCAACGGAGGAGGCAAGCAGGCCCTGGAGAC
GGTACAGAGGCTGCTGCCAGTGCTGTGCCAGGCCCACGGCCTAACACCAGAACAGGTGGTCGCT
ATTGCCTCTAACATTGGTGGCAAGCAGGCCCTGGAGACGGTACAGCGCCTGCTGCCCGTGCTGTG
CCAAGCCCACGGCCTAACGCCAGAACAGGTCGTCGCTATCGCCAGCAACGGAGGAGGCAAGCAG
GCCCTGGAGACGGTACAGCGGCTGCTGCCCGTGCTGTGCCAAGCCCACGGCCTAACCCCACAGCA
GGTCGTGGCCATTGCCTCCAATAACGGCGGCAAGCAGGCCCTGGAGACGGTACAGCGGCTGCTG
CCCGTGCTGTGCCAAGCCCACGGCCTAACTCCCCAGCAAGTCGTCGCTATTGCCTCTAATAACGG
GGGCAAGCAGGCCCTGGAGACGGTACAGAGACTGCTGCCCGTGCTGTGCCAAGCCCACGGCCTG
ACACCACAGCAGGTCGTCGCCATCGCAAGCAACGGAGGAGGGAGGCCCGCACTGGAGAGCATTG
TGGCGCAGCTGAGCCGTCCAGACCCGGCCCTGGCGGCTCTGACCAATGATCACCTGGTGGCCCTG
GCCTGCCTGGGTGGCCGTCCGGCTCTGGATGCCGTGAAGAAAGGTCTGGGC
Translated amino acid sequence:
(SEQ ID NO: 165)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPQQVVAIASNIGGKQALETVQRLLPVLCQAHGLTPEQVVAIASNIGGKQALETVQRLLPVL
CQAHGLTPQQVVAIASNNGGKQALETVQRLLPVLCQAHGLTPEQVVAIASNIGGKQALETVQALLPV
LCQAHGLTPQQVVAIASNGGGKQALETVQRLLPVLCQAHGLTPQQVVAIASNGGGKQALETVQRLLP
VLCQAHGLTPEQVVAIASNIGGKQALETVQRLLPVLCQAHGLTPQQVVAIASNNGGKQALETVQRLL
PVLCQAHGLTPQQVVAIASNGGGKQALETVQRLLPVLCQAHGLTPEQVVAIASNIGGKQALETVQRL
LPVLCQAHGLTPEQVVAIASNGGGKQALETVQRLLPVLCQAHGLTPQQVVAIASNNGGKQALETVQR
LLPVLCQAHGLTPQQVVAIASNNGGKQALETVQRLLPVLCQAHGLTPQQVVAIASNGGGRPALESIV
AQLSRPDPALAALTNDHLVALACLGGRPALDAVKKGLG
(R) ND5.3-DdCBE Left mitoTALE repeat
(SEQ ID NO: 166)
GACATCGCGGATCTGCGTACCCTGGGTTACAGCCAGCAGCAGCAGGAGAAGATCAAGCCGAAGG
TGCGCAGCACCGTGGCTCAGCACCACGAAGCCCTGGTGGGCCACGGTTTCACCCACGCTCACATT
GTGGCCCTGAGCCAGCACCCAGCCGCGCTGGGCACCGTGGCCGTGAAATATCAGGATATGATTGC
TGCCCTGCCAGAGGCCACCCATGAAGCTATTGTGGGCGTGGGCAAGCAGTGGAGCGGTGCTCGTG
CGCTGGAGGCGCTGCTGACCGTGGCTGGTGAACTGCGTGGTCCGCCGCTGCAGCTGGACACCGGT
CAGCTGCTGAAAATCGCGAAACGTGGCGGTGTGACCGCGGTGGAAGCCGTGCATGCTTGGCGTA
ATGCTCTGACCGGTGCGCCGCTGAACCTGACTCCCGAACAGGTGGTCGCTATCGCTTCTCATGAT
GGCGGAAAACAGGCTCTGGAAACCGTCCAGGCTCTGCTGCCCGTGCTGTGCCAGGCCCACGGCCT
GACCCCACAGCAGGTCGTCGCAATCGCCAGCAATATCGGAGGCAAGCAGGCCCTGGAGACCGTG
CAGCGGCTGCTGCCCGTGCTGTGCCAAGCCCACGGCTTAACACCTCAGCAGGTGGTGGCCATCGC
CTCCAACAATGGCGGCAAGCAGGCCCTGGAGACGGTGCAGAGACTGCTGCCAGTGCTGTGCCAG
GCCCACGGCTTAACACCAGAACAGGTCGTGGCAATCGCCTCTAACGGAGGGGGCAAGCAGGCCC
TGGAGACTGTGCAGGCCCTGCTGCCCGTGCTGTGCCAGGCCCACGGCCTTACACCAGAACAGGTG
GTCGCCATTGCCAGCAATGGAGGTGGCAAGCAGGCCCTGGAGACAGTCCAGGCCCTGCTGCCCGT
GCTGTGCCAAGCCCACGGCCTGACACCTGAACAGGTGGTCGCAATCGCCTCCCACGATGGGGGCA
AGCAGGCCCTGGAGACGGTACAGGCCCTGCTGCCCGTGCTGTGCCAAGCCCACGGCCTAACACCC
GAACAGGTGGTGGCCATTGCCTCTAACGGAGGAGGCAAGCAGGCCCTGGAGACGGTCCAGCGGC
TGCTGCCTGTGCTGTGCCAAGCCCACGGCCTTACCCCTGAACAAGTCGTGGCCATCGCCAGCAAT
GGAGGAGGCAAGCAGGCCCTGGAGACGGTTCAGGCCCTGCTGCCCGTGCTGTGCCAAGCCCACG
GCCTCACACCTGAACAAGTIGTGGCCATCGCCTCCCACGATGGTGGCAAGCAGGCCCTGGAGACG
GTACAGAGGCTGCTGCCAGTGCTGTGCCAGGCCCACGGCCTAACACCAGAACAGGTGGTGGCTAT
CGCCTCTAACATTGGCGGCAAGCAGGCCCTGGAGACGGTACAGGCCCTGCTGCCCGTGCTGTGCC
AAGCCCACGGCCTAACGCCAGAACAGGTCGTCGCTATTGCCAGCAACATTGGGGGCAAGCAGGC
CCTGGAGACGGTACAGGCCCTGCTGCCCGTGCTGTGCCAAGCCCACGGCCTAACCCCTGAACAGG
TGGTGGCAATCGCCTCCAACATTGGTGGCAAGCAGGCCCTGGAGACGGTACAGGCCCTGCTGCCC
GTGCTGTGCCAAGCCCACGGCCTAACTCCCGAGCAGGTCGTCGCCATCGCCTCTAATGGCGGCGG
CAAGCAGGCCCTGGAGACGGTACAGAGGCTGCTGCCTGTGCTGTGCCAAGCCCACGGCCTAACG
CCGCAGCAAGTCGTCGCTATTGCCAGCAATATTGGCGGCAAGCAGGCCCTGGAGACGGTACAGC
GCCTGCTGCCCGTGCTGTGCCAAGCCCACGGCCTGACCCCCCAGCAGGTGGTGGCAATCGCTTCA
AACGGAGGAGGGAGACCCGCTCTGGAAAGCATTGTGGCTCAGCTGAGCCGTCCAGACCCGGCCC
TGGCGGCTCTGACCAACGATCACCTGGTGGCGCTGGCTTGCCTGGGCGGTCGTCCGGCCCTGGAT
GCGGTGAAGAAAGGCCTGGGT
Translated amino acid sequence:
(SEQ ID NO: 167)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASHDGGKQALETVQALLPVLCQAHGLTPQQVVAIASNIGGKQALETVQRLLPVL
CQAHGLTPQQVVAIASNNGGKQALETVQRLLPVLCQAHGLTPEQVVAIASNGGGKQALETVQALLPV
LCQAHGLTPEQVVAIASNGGGKQALETVQALLPVLCQAHGLTPEQVVAIASHDGGKQALETVQALLP
VLCQAHGLTPEQVVAIASNGGGKQALETVQRLLPVLCQAHGLTPEQVVAIASNGGGKQALETVQALL
PVLCQAHGLTPEQVVAIASHDGGKQALETVQRLLPVLCQAHGLTPEQVVAIASNIGGKQALETVQAL
LPVLCQAHGLTPEQVVAIASNIGGKQALETVQALLPVLCQAHGLTPEQVVAIASNIGGKQALETVQAL
LPVLCQAHGLTPEQVVAIASNGGGKQALETVQRLLPVLCQAHGLTPQQVVAIASNIGGKQALETVQR
LLPVLCQAHGLTPQQVVAIASNGGGRPALESIVAQLSRPDPALAALTNDHLVALACLGGRPALDAVK
KGLG
(S) ATP8-DdCBE Right mitoTALE repeat
(SEQ ID NO: 168)
GACATCGCGGATCTGCGTACCCTGGGTTACAGCCAGCAGCAGCAGGAGAAGATCAAGCCAAAGG
TGCGCAGCACCGTGGCCCAGCACCATGAAGCTCTGGTGGGTCACGGCTTCACCCACGCGCACATC
GTGGCTCTGAGCCAGCACCCAGCCGCGCTGGGTACCGTGGCCGTGAAATATCAGGACATGATTGC
TGCCCTGCCAGAGGCTACCCATGAAGCGATTGTGGGTGTGGGCAAGCAGTGGAGCGGTGCTCGTG
CGCTGGAGGCCCTGCTGACCGTGGCCGGTGAACTGCGTGGCCCGCCGCTGCAGCTGGATACCGGC
CAGCTGCTGAAAATCGCGAAACGTGGCGGTGTGACCGCTGTGGAAGCTGTGCATGCCTGGCGTAA
TGCTCTGACCGGTGCCCCGCTGAACCTGACCCCTCAGCAGGTGGTGGCCATCGCCAGCAACATCG
GCGGCAAGCAGGCCCTGGAGACAGTGCAGAGGCTGCTGCCCGTGCTGTGCCAGGCACACGGCCT
GACACCTGAGCAGGTGGTGGCAATCGCATCCAATGGAGGAGGCAAGCAGGCCCTGGAGACAGTA
CAGCGCCTGTTACCCGTGCTGTGCCAGGCCCACGGCCTGACACCCCAGCAGGTCGTCGCCATCGC
CTCTAACAATGGGGGCAAGCAGGCCCTGGAGACAGTCCAGCGGCTGCTGCCTGTGCTGTGCCAGG
CTCATGGCCTGACACCAGAACAGGTCGTGGCTATTGCCAGCAACAATGGTGGCAAGCAGGCCCTG
GAGACAGTTCAGGCCCTGCTTCCCGTGCTGTGCCAGGCTCACGGCCTGACACCACAGCAGGTCGT
GGCCATCGCCTCCAACAATGGCGGCAAGCAGGCCCTGGAGACAGTACAGAGACTGCTGCCAGTG
CTGTGCCAAGCCCATGGCCTGACCCCTCAGCAGGTCGTGGCAATCGCATCTCACGACGGTGGCAA
GCAGGCCCTGGAGACAGTACAGAGGCTGTTACCCGTGCTGTGCCAAGCACACGGCCTGACACCA
GAGCAGGTCGTCGCAATCGCAAGCAACGGCGGCGGCAAGCAGGCCCTGGAGACAGTACAGCGCC
TGCTCCCCGTGCTGTGCCAAGCCCACGGCCTGACACCTCAGCAGGTGGTCGCCATTGCCAGCAAC
GGCGGGGGCAAGCAGGCCCTGGAGACAGTACAGCGGCTGTTGCCCGTGCTGTGCCAAGCCCACG
GCCTGACGCCCCAGCAGGTGGTCGCCATCGCATCTAACGGCGGTGGCAAGCAGGCCCTGGAGAC
AGTACAGCGGCTGCTTCCTGTGCTGTGCCAGGCCCATGGCCTGACCCCCGAACAGGTCGTGGCTA
TCGCTAGCAACAATGGCGGCAAGCAGGCCCTGGAGACAGTACAGAGACTGTTACCCGTGCTGTG
CCAAGCGCATGGCCTGACCCCTGAACAGGTCGTGGCAATTGCCTCCAATAACGGTGGCAAGCAG
GCCCTGGAGACAGTACAGCGGCTGCTACCAGTGCTGTGCCAAGCACATGGCCTGACCCCCCAGCA
GGTCGTGGCTATTGCATCTAATGGAGGAGGCAGACCCGCCCTGGAGAGCATTGTGGCGCAGCTGA
GCCGTCCAGACCCGGCCCTGGCGGCTCTGACCAATGATCACCTGGTGGCCCTGGCCTGCCTGGGT
GGCCGTCCGGCTCTGGATGCCGTGAAGAAAGGTCTGGGC
Translated amino acid sequence:
(SEQ ID NO: 12)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPQQVVAIASNIGGKQALETVQRLLPVLCQAHGLTPEQVVAIASNGGGKQALETVQRLLPVL
CQAHGLTPQQVVAIASNNGGKQALETVQRLLPVLCQAHGLTPEQVVAIASNNGGKQALETVQALLPV
LCQAHGLTPQQVVAIASNNGGKQALETVQRLLPVLCQAHGLTPQQVVAIASHDGGKQALETVQRLLP
VLCQAHGLTPEQVVAIASNGGGKQALETVQRLLPVLCQAHGLTPQQVVAIASNGGGKQALETVQRLL
PVLCQAHGLTPQQVVAIASNGGGKQALETVQRLLPVLCQAHGLTPEQVVAIASNNGGKQALETVQRL
LPVLCQAHGLTPEQVVAIASNNGGKQALETVQRLLPVLCQAHGLTPQQVVAIASNGGGRPALESIVA
QLSRPDPALAALTNDHLVALACLGGRPALDAVKKGLG
(T) ATP8-DdCBE Left mitoTALE repeat
(SEQ ID NO: 169)
GACATCGCGGATCTGCGTACCCTGGGTTACAGCCAGCAGCAGCAGGAGAAGATCAAGCCGAAGG
TGCGCAGCACCGTGGCTCAGCACCACGAAGCCCTGGTGGGCCACGGTTTCACCCACGCTCACATC
GTGGCCCTGAGCCAGCACCCAGCCGCGCTGGGCACCGTGGCCGTGAAATATCAGGACATGATTGC
TGCCCTGCCAGAGGCCACCCATGAAGCTATTGTGGGCGTGGGCAAGCAGTGGAGCGGTGCTCGTG
CGCTGGAGGCGCTGCTGACCGTGGCTGGTGAACTGCGTGGTCCGCCGCTGCAGCTGGATACCGGT
CAGCTGCTGAAAATCGCGAAACGTGGCGGTGTGACCGCGGTGGAAGCCGTGCATGCTTGGCGTA
ATGCTCTGACCGGTGCGCCGCTGAACCTGACCCCGGAGCAGGTGGTGGCTATCGCCAGCAACATT
GGCGGTAAACAGGCCCTGGAAACCGTGCAGGCGCTGCTGCCGGTGCTGTGCCAGGCTCATGGTCT
GACCCCGCAGCAGGTGGTGGCGATCGCTAGCAACGGCGGTGGCAAGCAGGCTCTGGAGACCGTG
CAGCGTCTGTTACCGGTGCTGTGCCAAGCCCATGGCCTGACCCCGCAGCAAGTTGTGGCCATTGC
GAGCAATGGTGGCGGTAAACAGGCGCTGGAAACCGTGCAGCGCCTGTTGCCGGTGCTGTGCCAA
GCCCATGGGCTGACCCCGGAACAAGTTGTTGCTATCGCCAGCAACATCGGCGGCAAGCAGGCTCT
GGAAACCGTGCAGGCCCTGCTTCCGGTGCTGTGCCAAGCGCATGGTCTGACCCCGGAACAAGTGG
TGGCCATCGCTTCCAATATTGGCGGTAAACAGGCGCTGGAGACCGTGCAGGCTCTGCTCCCGGTG
CTGTGCCAAGCACATGGTCTGACCCCGGAGCAAGTTGTGGCTATTGCCTCCAATATCGGCGGCAA
GCAGGCCCTGGAGACCGTTCAGGCGCTGTTACCGGTGCTGTGCCAGGCCCATGGATTAACCCCGG
AGCAAGTGGTGGCTATTGCTAGCCATGATGGCGGTAAACAGGCCCTGGAGACTGTTCAGCGTCTG
CTACCGGTGCTGTGCCAGGCCCATGGTTTAACCCCGGAACAGGTTGTTGCCATCGCTTCCAACATC
GGCGGCAAGCAGGCTCTGGAAACGGTGCAGGCCCTGTTACCGGTGCTGTGCCAGGCCCATGGGTT
AACCCCGGAACAAGTTGTGGCCATTGCCTCCCATGACGGCGGTAAACAGGCTCTGGAGACCGTTC
AGCGCCTGCTACCGGTGCTGTGCCAGGCGCATGGCTTAACCCCGGAACAAGTGGTTGCCATTGCG
TCCAATATCGGCGGCAAGCAGGCGCTGGAGACCGTTCAGGCTCTGCTTCCGGTGCTGTGCCAGGC
ACATGGCCTTACCCCGGAACAAGTGGTCGCGATCGCTTCCAACATTGGCGGTAAACAGGCCCTGG
AAACGGTTCAGGCGCTGCTTCCGGTGCTGTGCCAGGCCCATGGGCTTACCCCGGAACAGGTTGTG
GCTATTGCCAGTAATATCGGCGGCAAGCAGGCTCTGGAAACTGTGCAGGCCCTGCTACCGGTGCT
GTGCCAGGCTCATGGGCTGACCCCGGAGCAAGTGGTTGCCATTGCCTCCCATGATGGCGGTAAAC
AGGCGCTGGAAACGGTGCAGCGTCTGCTTCCGGTGCTGTGCCAGGCTCATGGCTTAACCCCGCAG
CAAGTTGTTGCGATTGCTAGCAATGGCGGTGGCAAGCAGGCCCTGGAAACTGTTCAGCGCCTGTT
ACCGGTGCTGTGCCAGGCCCATGGGCTAACCCCGGAACAGGTGGTTGCTATTGCCAGCAACATTG
GTGGCAAACAGGCGCTGGAAACTGTGCAGGCTCTGCTTCCGGTGCTGTGCCAGGCCCATGGGCTG
ACCCCGCAGCAAGTGGTTGCTATTGCTAGCAATGGTGGCGGTCGTCCGGCCCTGGAGAGCATTGT
GGCGCAGCTGAGCCGTCCAGACCCGGCCCTGGCGGCTCTGACCAACGATCACCTGGTGGCGCTGG
CTTGCCTGGGCGGTCGTCCGGCCCTGGATGCGGTGAAGAAAGGCCTGGGT
Translated amino acid sequence:
(SEQ ID NO: 170)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASNIGGKQALETVQALLPVLCQAHGLTPQQVVAIASNGGGKQALETVQRLLPVL
CQAHGLTPQQVVAIASNGGGKQALETVQRLLPVLCQAHGLTPEQVVAIASNIGGKQALETVQALLPV
LCQAHGLTPEQVVAIASNIGGKQALETVQALLPVLCQAHGLTPEQVVAIASNIGGKQALETVQALLPV
LCQAHGLTPEQVVAIASHDGGKQALETVQRLLPVLCQAHGLTPEQVVAIASNIGGKQALETVQALLP
VLCQAHGLTPEQVVAIASHDGGKQALETVQRLLPVLCQAHGLTPEQVVAIASNIGGKQALETVQALL
PVLCQAHGLTPEQVVAIASNIGGKQALETVQALLPVLCQAHGLTPEQVVAIASNIGGKQALETVQALL
PVLCQAHGLTPEQVVAIASHDGGKQALETVQRLLPVLCQAHGLTPQQVVAIASNGGGKQALETVQRL
LPVLCQAHGLTPEQVVAIASNIGGKQALETVQALLPVLCQAHGLTPQQVVAIASNGGGRPALESIVAQ
LSRPDPALAALTNDHLVALACLGGRPALDAVKKGLG

### Intact DddAtox-Cas9 Fusions Sequences

All intact DddAtox-Cas9 have the general architecture of (from N- to C-terminus): bpNLS-DddAtox-linker 1-dSpCas9 or SpCas9(D10A)-10aa-linker-10aa linker-UGI-4aa linker-bpNLS

(A) DddAtox
(SEQ IN NO: 25)
GSYALGPYQISAPQLPAYNGQTVGTFYYVNDAGGLESKVFSSGGPTPYPNYANAGHVEGQSALFMRD
NGISEGLVFHNNPEGTCGFCVNMTETLLPENAKMTVVPPEGAIPVKRGATGETKVFTGNSNSPKSPTK
GGC
(B) dSpCas9
(SEQ ID NO: 127)
DKKYSIGLAIGTNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNFDLAEDAKLQLSKDTYDDDL
DNLLAQIGDQYADLFLAAKNLSDAILLSDILRVNTEITKAPLSASMIKRYDEHHQDLTLLKALVRQQLP
EKYKEIFFDQSKNGYAGYIDGGASQEEFYKFIKPILEKMDGTEELLVKLNREDLLRKQRTFDNGSIPHQ
IHLGELHAILRRQEDFYPFLKDNREKIEKILTFRIPYYVGPLARGNSRFAWMTRKSEETITPWNFEEVVD
KGASAQSFIERMTNFDKNLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKIIKDKDFLDNEENEDILEDIV
LTLTLFEDREMIEERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKVVDELVKVMGRHK
PENIVIEMARENQTTQKGQKNSRERMKRIEEGIKELGSQILKEHPVENTQLQNEKLYLYYLQNGRDMY
VDQELDINRLSDYDVDAIVPQSFLKDDSIDNKVLTRSDKNRGKSDNVPSEEVVKKMKNYWRQLLNA
KLITQRKFDNLTKAERGGLSELDKAGFIKRQLVETRQITKHVAQILDSRMNTKYDENDKLIREVKVITL
KSKLVSDFRKDFQFYKVREINNYHHAHDAYLNAVVGTALIKKYPKLESEFVYGDYKVYDVRKMIAK
SEQEIGKATAKYFFYSNIMNFFKTEITLANGEIRKRPLIETNGETGEIVWDKGRDFATVRKVLSMPQVN
IVKKTEVQTGGFSKESILPKRNSDKLIARKKDWDPKKYGGFDSPTVAYSVLVVAKVEKGKSKKLKSV
KELLGITIMERSSFEKNPIDFLEAKGYKEVKKDLIIKLPKYSLFELENGRKRMLASAGELQKGNELALPS
KYVNFLYLASHYEKLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGLYETRIDLSQLGGD
(C) SpCas9 (D10A)
(SEQ ID NO: 128)
DKKYSIGLAIGTNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNFDLAEDAKLQLSKDTYDDDL
DNLLAQIGDQYADLFLAAKNLSDAILLSDILRVNTEITKAPLSASMIKRYDEHHQDLTLLKALVRQQLP
EKYKEIFFDQSKNGYAGYIDGGASQEEFYKFIKPILEKMDGTEELLVKLNREDLLRKQRTFDNGSIPHQ
IHLGELHAILRRQEDFYPFLKDNREKIEKILTFRIPYYVGPLARGNSRFAWMTRKSEETITPWNFEEVVD
KGASAQSFIERMTNFDKNLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKIIKDKDFLDNEENEDILEDIV
LTLTLFEDREMIEERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKVVDELVKVMGRHK
PENIVIEMARENQTTQKGQKNSRERMKRIEEGIKELGSQILKEHPVENTQLQNEKLYLYYLQNGRDMY
VDQELDINRLSDYDVDHIVPQSFLKDDSIDNKVLTRSDKNRGKSDNVPSEEVVKKMKNYWRQLLNA
KLITQRKFDNLTKAERGGLSELDKAGFIKRQLVETRQITKHVAQILDSRMNTKYDENDKLIREVKVITL
KSKLVSDFRKDFQFYKVREINNYHHAHDAYLNAVVGTALIKKYPKLESEFVYGDYKVYDVRKMIAK
SEQEIGKATAKYFFYSNIMNFFKTEITLANGEIRKRPLIETNGETGEIVWDKGRDFATVRKVLSMPQVN
IVKKTEVQTGGFSKESILPKRNSDKLIARKKDWDPKKYGGFDSPTVAYSVLVVAKVEKGKSKKLKSV
KELLGITIMERSSFEKNPIDFLEAKGYKEVKKDLIIKLPKYSLFELENGRKRMLASAGELQKGNELALPS
KYVNFLYLASHYEKLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGLYETRIDLSQLGGD
(D) UGI
(SEQ ID NO: 378)
TNLSDIIEKETGKQLVIQESILMLPEEVEEVIGNKPESDILVHTAYDESTDENVMLLTSDAPEYKPWALV
IQDSNGENKIKML
(E) bpNLS
(SEQ ID NO: 234)
KRTADGSEFEPKKKRKV
(F) Linker 1: 32 aa linker
(SEQ ID NO: 203)
SGGSSGGSSGSETPGTSESATPESSGGSSGGS
(G) Linker 1: 10 aa flexible
(SEQ ID NO: 218)
GGGGSGGGGS
(H) Linker 1: 10 aa rigid
(SEQ ID NO: 208)
EAAAKEAAAK
(I) Linker 1: 5 aa rigid
(SEQ ID NO: 219)
EAAAK
(J) 10 aa linker
(SEQ ID NO: 220)
SGGSGGSGGS
(K) aa linker
(SEQ ID NO: 221)
SGGS

### Split-DddAtox-Cas9 Fusions Sequences

All split-DddAtox-Cas9 have the general architecture of (from N- to C-terminus): bpNLS-DddAtox half-32aa linker-dSpCas9 or SaKKH-Cas9(D10A)-10aa linker-UGI-10aa linker-UGI-4aa linker-bpNLS

(A) G1333 DddAtox-N¬
(SEQ ID NO: 338)
GSYALGPYQISAPQLPAYNGQTVGTFYYVNDAGGLESKVFSSGG
(B) G1333 DddAtox-C
(SEQ ID NO: 339)
PTPYPNYANAGHVEGQSALFMRDNGISEGLVFHNNPEGTCGFCVNMTETLLPENAKMTVVPPEGAIP
VKRGATGETKVFTGNSNSPKSPTKGGC
(C) G1397 DddAtox-N¬
(SEQ ID NO: 340)
GSYALGPYQISAPQLPAYNGQTVGTFYYVNDAGGLESKVFSSGGPTPYPNYANAGHVEGQSALFMRD
NGISEGLVFHNNPEGTCGFCVNMTETLLPENAKMTVVPPEG
(D) G1397 DddAtox-C
(SEQ ID NO: 341)
AIPVKRGATGETKVFTGNSNSPKSPTKGGC
(E) dSpCas9
(SEQ ID NO: 127)
DKKYSIGLAIGTNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNFDLAEDAKLQLSKDTYDDDL
DNLLAQIGDQYADLFLAAKNLSDAILLSDILRVNTEITKAPLSASMIKRYDEHHQDLTLLKALVRQQLP
EKYKEIFFDQSKNGYAGYIDGGASQEEFYKFIKPILEKMDGTEELLVKLNREDLLRKQRTFDNGSIPHQ
IHLGELHAILRRQEDFYPFLKDNREKIEKILTFRIPYYVGPLARGNSRFAWMTRKSEETITPWNFEEVVD
KGASAQSFIERMTNFDKNLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKIIKDKDFLDNEENEDILEDIV
LTLTLFEDREMIEERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKVVDELVKVMGRHK
PENIVIEMARENQTTQKGQKNSRERMKRIEEGIKELGSQILKEHPVENTQLQNEKLYLYYLQNGRDMY
VDQELDINRLSDYDVDAIVPQSFLKDDSIDNKVLTRSDKNRGKSDNVPSEEVVKKMKNYWRQLLNA
KLITQRKFDNLTKAERGGLSELDKAGFIKRQLVETRQITKHVAQILDSRMNTKYDENDKLIREVKVITL
KSKLVSDFRKDFQFYKVREINNYHHAHDAYLNAVVGTALIKKYPKLESEFVYGDYKVYDVRKMIAK
SEQEIGKATAKYFFYSNIMNFFKTEITLANGEIRKRPLIETNGETGEIVWDKGRDFATVRKVLSMPQVN
IVKKTEVQTGGFSKESILPKRNSDKLIARKKDWDPKKYGGFDSPTVAYSVLVVAKVEKGKSKKLKSV
KELLGITIMERSSFEKNPIDFLEAKGYKEVKKDLIIKLPKYSLFELENGRKRMLASAGELQKGNELALPS
KYVNFLYLASHYEKLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGLYETRIDLSQLGGD
(F)
SaKKH-Cas9(D10A)
(SEQ ID NO: 129)
GKRNYILGLAIGITSVGYGIIDYETRDVIDAGVRLFKEANVENNEGRRSKRGARRLKRRRRHRIQRVK
KLLFDYNLLTDHSELSGINPYEARVKGLSQKLSEEEFSAALLHLAKRRGVHNVNEVEEDTGNELSTKE
QISRNSKALEEKYVAELQLERLKKDGEVRGSINRFKTSDYVKEAKQLLKVQKAYHQLDQSFIDTYIDL
LETRRTYYEGPGEGSPFGWKDIKEWYEMLMGHCTYFPEELRSVKYAYNADLYNALNDLNNLVITRD
ENEKLEYYEKFQIIENVFKQKKKPTLKQIAKEILVNEEDIKGYRVTSTGKPEFTNLKVYHDIKDITARKE
IIENAELLDQIAKILTIYQSSEDIQEELTNLNSELTQEEIEQISNLKGYTGTHNLSLKAINLILDELWHTND
NQIAIFNRLKLVPKKVDLSQQKEIPTTLVDDFILSPVVKRSFIQSIKVINAIIKKYGLPNDIIIELAREKNSK
DAQKMINEMQKRNRQTNERIEEIIRTTGKENAKYLIEKIKLHDMQEGKCLYSLEAIPLEDLLNNPFNYE
VDHIIPRSVSFDNSFNNKVLVKQEENSKKGNRTPFQYLSSSDSKISYETFKKHILNLAKGKGRISKTKKE
YLLEERDINRFSVQKDFINRNLVDTRYATRGLMNLLRSYFRVNNLDVKVKSINGGFTSFLRRKWKFK
KERNKGYKHHAEDALIIANADFIFKEWKKLDKAKKVMENQMFEEKQAESMPEIETEQEYKEIFITPHQ
IKHIKDFKDYKYSHRVDKKPNRKLINDTLYSTRKDDKGNTLIVNNLNGLYDKDNDKLKKLINKSPEKL
LMYHHDPQTYQKLKLIMEQYGDEKNPLYKYYEETGNYLTKYSKKDNGPVIKKIKYYGNKLNAHLDI
TDDYPNSRNKVVKLSLKPYRFDVYLDNGVYKFVTVKNLDVIKKENYYEVNSKCYEEAKKLKKISNQ
AEFIASFYKNDLIKINGELYRVIGVNNDLLNRIEVNMIDITYREYLENMNDKRPPHIIKTIASKTQSIKKY
STDILGNLYEVKSKKHPQIIKKGGSPKKKRKVSSDYKDHDGDYKDHDIDYKDDDDK
(G) UGI
(SEQ ID NO: 378)
TNLSDIIEKETGKQLVIQESILMLPEEVEEVIGNKPESDILVHTAYDESTDENVMLLTSDAPEYKPWALV
IQDSNGENKIKML
(H) bpNLS
(SEQ ID NO: 234)
KRTADGSEFEPKKKRKV
(I) 32 aa linker
(SEQ ID NO: 203)
SGGSSGGSSGSETPGTSESATPESSGGSSGGS
(J) 10 aa linker
(SEQ ID NO: 220)
SGGSGGSGGS
(K) 4 aa linker
(SEQ ID NO: 221)
SGGS

General DdCBE Architecture and mitoTALE Amino Acid Sequences

All right-side halves of DdCBEs have the general architecture of (from N- to C-terminus): COX8A MTS-3×FLAG-mitoTALE-2aa linker-DddAtox half-4aa linker-1×-UGI-ATP5B 3′UTR.

All left-side halves of DdCBEs have the general architecture of (from N- to C-terminus): SOD2 MTS-3×HA-mitoTALE-2aa linker-DddAtox half-4aa linker-1×-UGI-SOD2 3′UTR

mitoTALE domains are annotated as: bold for N-terminal domain, underlined for RVD and bolded italics for C-terminal domain.

ND6-DdCBE: Left mitoTALE-G1397-DddAtox-N-1x-UGI (Note: Terminal
NG RVD recognizes a mismatched T instead of a G in the reference
genome)
(SEQ ID NO: 5)
MALSRAVCGTSRQLAPVLGYLGSRQKHSLPDYPYDVPDYAGYPYDVPDYAGYPYDVPDYAMDIADL
RTLGYSQQQQEKIKPKVRSTVAQHHEALVGHGFTHAHIVALSQHPAALGTVAVKYQDMIAALPEATH
EAIVGVGKQWSGARALEALLTVAGELRGPPLQLDTGQLLKIAKRGGVTAVEAVHAWRNALTGAPLN
LTPQQVVAIASNNGGKQALETVQRLLPVLCQAHGLTPEQVVAIASNIGGKQALETVQALLPVLCQAH
GLTPEQVVAIASHDGGKQALETVQRLLPVLCQAHGLTPEQVVAIASHDGGKQALETVQRLLPVLCQA
HGLTPEQVVAIASHDGGKQALETVQRLLPVLCQAHGLTPEQVVAIASHDGGKQALETVQRLLPVLCQ
AHGLTPEQVVAIASHDGGKQALETVQRLLPVLCQAHGLTPEQVVAIASNIGGKQALETVQALLPVLC
QAHGLTPQQVVAIASNGGGKQALETVQRLLPVLCQAHGLTPQQVVAIASNGGGRPALESIVAQLSRP
DPALAALTNDHLVALACLGGRPALDAVKKGLGGSGSYALGPYQISAPQLPAYNGQTVGTFYYVNDA
GGLESKVFSSGGPTPYPNYANAGHVEGQSALFMRDNGISEGLVFHNNPEGTCGFCVNMTETLLPENA
KMTVVPPEGSGGSTNLSDIIEKETGKQLVIQESILMLPEEVEEVIGNKPESDILVHTAYDESTDENVMLL
TSDAPEYKPWALVIQDSNGENKIKML**
ND6-DdCBE: Right mitoTALE-G1397-DddAtox-N-1x-UGI (Note: Terminal
NG RVD recognizes a mismatched T instead of a G in the reference
genome. The NTD was also engineered to be permissive for A, T,
C and G nucleotides at the N0 position)
(SEQ ID NO: 10)
MASVLTPLLLRGLTGSARRLPVPRAKIHSLDYKDHDGDYKDHDIDYKDDDDKMDIADLRTLGYSQQ
QQEKIKPKVRSTVAQHHEALVGHGFTHAHIVALSQHPAALGTVAVKYQDMIAALPEATHEAIVGVGK
RGAGARALEALLTVAGELRGPPLQLDTGQLLKIAKRGGVTAVEAVHAWRNALTGAPLNLTPQQVVA
IASNNGGKQALETVQRLLPVLCQAHGLTPEQVVAIASNIGGKQALETVQALLPVLCQAHGLTPQQVV
AIASNGGGKQALETVQRLLPVLCQAHGLTPQQVVAIASNNGGKQALETVQRLLPVLCQAHGLTPQQV
VAIASNNGGKQALETVQRLLPVLCQAHGLTPEQVVAIASHDGGKQALETVQRLLPVLCQAHGLTPQQ
VVAIASNGGGKQALETVQRLLPVLCQAHGLTPEQVVAIASNIGGKQALETVQALLPVLCQAHGLTPQ
QVVAIASNGGGKQALETVQRLLPVLCQAHGLTPQQVVAIASNGGGKQALETVQRLLPVLCQAHGLTP
QQVVAIASNGGGRPALESIVAQLSRPDPALAALTNDHLVALACLGGRPALDAVKKGLGGSAIPVKRG
ATGETKVFTGNSNSPKSPTKGGCSGGSTNLSDIIEKETGKQLVIQESILMLPEEVEEVIGNKPESDILVHT
AYDESTDENVMLLTSDAPEYKPWALVIQDSNGENKIKML**
ND1-DdCBE Right mitoTALE repeat
(SEQ ID NO: 147)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASNNGGKQALETVQALLPVLCQAHGLTPQQVVAIASNIGGKQALETVQRLLPVL
CQAHGLTPQQVVAIASNNGGKQALETVQRLLPVLCQAHGLTPEQVVAIASNGGGKQALETVQALLPV
LCQAHGLTPEQVVAIASNGGGKQALETVQALLPVLCQAHGLTPEQVVAIASNGGGKQALETVQALLP
VLCQAHGLTPEQVVAIASNNGGKQALETVQRLLPVLCQAHGLTPEQVVAIASNIGGKQALETVQALL
PVLCQAHGLTPEQVVAIASNGGGKQALETVQRLLPVLCQAHGLTPEQVVAIASNNGGKQALETVQAL
LPVLCQAHGLTPEQVVAIASHDGGKQALETVQALLPVLCQAHGLTPEQVVAIASNGGGKQALETVQA
LLPVLCQAHGLTPEQVVAIASHDGGKQALETVQRLLPVLCQAHGLTPQQVVAIASNIGGKQALETVQ
RLLPVLCQAHGLTPEQVVAIASHDGGKQALETVQALLPVLCQAHGLTPQQVVAIASHDGGKQALETV
QRLLPVLCQAHGLTPQQVVAIASHDGGKQALETVQRLLPVLCQAHGLTPQQVVAIASNGGGRPALESI
VAQLSRPDPALAALTNDHLVALACLGGRPALDAVKKGLG
ND1-DdCBE Left mitoTALE repeat
(SEQ ID NO: 149)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASHDGGKQALETVQALLPVLCQAHGLTPQQVVAIASNGGGKQALETVQRLLPV
LCQAHGLTPQQVVAIASNIGGKQALETVQRLLPVLCQAHGLTPEQVVAIASNNGGKQALETVQALLP
VLCQAHGLTPEQVVAIASHDGGKQALETVQALLPVLCQAHGLTPEQVVAIASHDGGKQALETVQALL
PVLCQAHGLTPEQVVAIASNGGGKQALETVQRLLPVLCQAHGLTPEQVVAIASNIGGKQALETVQAL
LPVLCQAHGLTPEQVVAIASNNGGKQALETVQRLLPVLCQAHGLTPEQVVAIASHDGGKQALETVQA
LLPVLCQAHGLTPEQVVAIASHDGGKQALETVQALLPVLCQAHGLTPEQVVAIASNNGGKQALETVQ
ALLPVLCQAHGLTPEQVVAIASNGGGKQALETVQRLLPVLCQAHGLTPQQVVAIASNGGGKQALETV
QRLLPVLCQAHGLTPQQVVAIASNGGGRPALESIVAQLSRPDPALAALTNDHLVALACLGGRPALDA
VKKGLG
ND2-DdCBE Right mitoTALE repeat
(SEQ ID NO: 151)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASNIGGKQALETVQALLPVLCQAHGLTPQQVVAIASNNGGKQALETVQRLLPVL
CQAHGLTPQQVVAIASNIGGKQALETVQRLLPVLCQAHGLTPEQVVAIASNIGGKQALETVQALLPVL
CQAHGLTPEQVVAIASHDGGKQALETVQALLPVLCQAHGLTPEQVVAIASNGGGKQALETVQALLPV
LCQAHGLTPEQVVAIASNNGGKQALETVQRLLPVLCQAHGLTPEQVVAIASHDGGKQALETVQALLP
VLCQAHGLTPEQVVAIASNGGGKQALETVQRLLPVLCQAHGLTPEQVVAIASNIGGKQALETVQALL
PVLCQAHGLTPEQVVAIASNGGGKQALETVQALLPVLCQAHGLTPEQVVAIASNGGGKQALETVQAL
LPVLCQAHGLTPEQVVAIASNIGGKQALETVQRLLPVLCQAHGLTPQQVVAIASNGGGKQALETVQR
LLPVLCQAHGLTPQQVVAIASNGGGRPALESIVAQLSRPDPALAALTNDHLVALACLGGRPALDAVK
KGLG
ND2-DdCBE Left mitoTALE repeat
(SEQ ID NO: 171)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASHDGGKQALETVQALLPVLCQAHGLTPQQVVAIASNGGGKQALETVQRLLPV
LCQAHGLTPQQVVAIASNGGGKQALETVQRLLPVLCQAHGLTPEQVVAIASNIGGKQALETVQALLP
VLCQAHGLTPEQVVAIASNNGGKQALETVQALLPVLCQAHGLTPEQVVAIASHDGGKQALETVQALL
PVLCQAHGLTPEQVVAIASNIGGKQALETVQRLLPVLCQAHGLTPEQVVAIASNGGGKQALETVQAL
LPVLCQAHGLTPEQVVAIASNIGGKQALETVQRLLPVLCQAHGLTPEQVVAIASHDGGKQALETVQA
LLPVLCQAHGLTPEQVVAIASNGGGKQALETVQALLPVLCQAHGLTPEQVVAIASHDGGKQALETVQ
ALLPVLCQAHGLTPEQVVAIASHDGGKQALETVQRLLPVLCQAHGLTPQQVVAIASNGGGKQALETV
QRLLPVLCQAHGLTPEQVVAIASHDGGKQALETVQALLPVLCQAHGLTPEQVVAIASNIGGKQALET
VQALLPVLCQAHGLTPEQVVAIASNIGGKQALETVQALLPVLCQAHGLTPQQVVAIASNGGGRPALES
IVAQLSRPDPALAALTNDHLVALACLGGRPALDAVKKGLG
ND4-DdCBE Right mitoTALE repeat
(SEQ ID NO: 154)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASHDGGKQALETVQALLPVLCQAHGLTPQQVVAIASHDGGKQALETVQRLLPV
LCQAHGLTPQQVVAIASNGGGKQALETVQRLLPVLCQAHGLTPEQVVAIASNNGGKQALETVQALLP
VLCQAHGLTPEQVVAIASNGGGKQALETVQALLPVLCQAHGLTPEQVVAIASNIGGKQALETVQALL
PVLCQAHGLTPEQVVAIASNIGGKQALETVQRLLPVLCQAHGLTPEQVVAIASNNGGKQALETVQAL
LPVLCQAHGLTPEQVVAIASNGGGKQALETVQRLLPVLCQAHGLTPEQVVAIASNIGGKQALETVQR
LLPVLCQAHGLTPEQVVAIASNNGGKQALETVQALLPVLCQAHGLTPEQVVAIASNNGGKQALETVQ
ALLPVLCQAHGLTPEQVVAIASNIGGKQALETVQALLPVLCQAHGLTPEQVVAIASNNGGKQALETV
QRLLPVLCQAHGLTPQQVVAIASNIGGKQALETVQRLLPVLCQAHGLTPQQVVAIASNNGGKQALET
VQRLLPVLCQAHGLTPQQVVAIASNGGGRPALESIVAQLSRPDPALAALTNDHLVALACLGGRPALD
AVKKGLG
ND4-DdCBE Left mitoTALE repeat
(SEQ ID NO: 156)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASNNGGKQALETVQALLPVLCQAHGLTPQQVVAIASHDGGKQALETVQRLLPV
LCQAHGLTPQQVVAIASNGGGKQALETVQRLLPVLCQAHGLTPEQVVAIASNIGGKQALETVQALLP
VLCQAHGLTPEQVVAIASNNGGKQALETVQALLPVLCQAHGLTPEQVVAIASNGGGKQALETVQALL
PVLCQAHGLTPEQVVAIASNIGGKQALETVQRLLPVLCQAHGLTPEQVVAIASNIGGKQALETVQALL
PVLCQAHGLTPEQVVAIASHDGGKQALETVQRLLPVLCQAHGLTPEQVVAIASHDGGKQALETVQAL
LPVLCQAHGLTPEQVVAIASNIGGKQALETVQALLPVLCQAHGLTPEQVVAIASHDGGKQALETVQA
LLPVLCQAHGLTPEQVVAIASNNGGKQALETVQRLLPVLCQAHGLTPQQVVAIASNGGGKQALETVQ
RLLPVLCQAHGLTPEQVVAIASNGGGKQALETVQALLPVLCQAHGLTPEQVVAIASHDGGKQALETV
QALLPVLCQAHGLTPQQVVAIASNGGGRPALESIVAQLSRPDPALAALTNDHLVALACLGGRPALDA
VKKGLG
ND5.1-DdCBE Right mitoTALE repeat
(SEQ ID NO: 11)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPQQVVAIASHDGGKQALETVQRLLPVLCQAHGLTPEQVVAIASNGGGKQALETVQRLLPV
LCQAHGLTPQQVVAIASNGGGKQALETVQRLLPVLCQAHGLTPEQVVAIASNGGGKQALETVQALLP
VLCQAHGLTPQQVVAIASNNGGKQALETVQRLLPVLCQAHGLTPQQVVAIASNNGGKQALETVQRL
LPVLCQAHGLTPEQVVAIASNIGGKQALETVQRLLPVLCQAHGLTPQQVVAIASNNGGKQALETVQR
LLPVLCQAHGLTPQQVVAIASNGGGKQALETVQRLLPVLCQAHGLTPEQVVAIASNIGGKQALETVQ
RLLPVLCQAHGLTPEQVVAIASNNGGKQALETVQRLLPVLCQAHGLTPQQVVAIASNIGGKQALETV
QRLLPVLCQAHGLTPQQVVAIASNIGGRPALESIVAQLSRPDPALAALTNDHLVALACLGGRPALDAV
KKGLG
ND5.1-DdCBE Left mitoTALE repeat
(SEQ ID NO: 172)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASNIGGKQALETVQALLPVLCQAHGLTPQQVVAIASNNGGKQALETVQRLLPVL
CQAHGLTPQQVVAIASHDGGKQALETVQRLLPVLCQAHGLTPEQVVAIASNIGGKQALETVQALLPV
LCQAHGLTPEQVVAIASNGGGKQALETVQALLPVLCQAHGLTPEQVVAIASNGGGKQALETVQALLP
VLCQAHGLTPEQVVAIASNIGGKQALETVQRLLPVLCQAHGLTPEQVVAIASNNGGKQALETVQALL
PVLCQAHGLTPEQVVAIASHDGGKQALETVQRLLPVLCQAHGLTPEQVVAIASNIGGKQALETVQAL
LPVLCQAHGLTPEQVVAIASNNGGKQALETVQALLPVLCQAHGLTPEQVVAIASNNGGKQALETVQA
LLPVLCQAHGLTPEQVVAIASNIGGKQALETVQRLLPVLCQAHGLTPQQVVAIASNIGGKQALETVQR
LLPVLCQAHGLTPQQVVAIASNGGGRPALESIVAQLSRPDPALAALTNDHLVALACLGGRPALDAVK
KGLG
ND5.2-DdCBE Right mitoTALE repeat (Note: Terminal NG RVD recognizes
a mismatched T instead of a G in the reference genome)
(SEQ ID NO: 161)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPQQVVAIASNNGGKQALETVQRLLPVLCQAHGLTPEQVVAIASHDGGKQALETVQRLLPV
LCQAHGLTPQQVVAIASNGGGKQALETVQRLLPVLCQAHGLTPEQVVAIASNIGGKQALETVQALLP
VLCQAHGLTPQQVVAIASNNGGKQALETVQRLLPVLCQAHGLTPQQVVAIASNNGGKQALETVQRL
LPVLCQAHGLTPEQVVAIASHDGGKQALETVQRLLPVLCQAHGLTPQQVVAIASNGGGKQALETVQR
LLPVLCQAHGLTPQQVVAIASNNGGKQALETVQRLLPVLCQAHGLTPEQVVAIASHDGGKQALETVQ
RLLPVLCQAHGLTPEQVVAIASHDGGKQALETVQRLLPVLCQAHGLTPEQVVAIASNIGGKQALETV
QALLPVLCQAHGLTPEQVVAIASNIGGKQALETVQALLPVLCQAHGLTPQQVVAIASNGGGKQALET
VQRLLPVLCQAHGLTPQQVVAIASNNGGKQALETVQRLLPVLCQAHGLTPQQVVAIASNGGGRPALE
SIVAQLSRPDPALAALTNDHLVALACLGGRPALDAVKKGLG
ND5.2-DdCBE Left mitoTALE repeat
(SEQ ID NO: 173)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASHDGGKQALETVQALLPVLCQAHGLTPQQVVAIASNIGGKQALETVQRLLPVL
CQAHGLTPQQVVAIASNIGGKQALETVQRLLPVLCQAHGLTPEQVVAIASNIGGKQALETVQALLPVL
CQAHGLTPEQVVAIASNIGGKQALETVQALLPVLCQAHGLTPEQVVAIASHDGGKQALETVQALLPV
LCQAHGLTPEQVVAIASHDGGKQALETVQRLLPVLCQAHGLTPEQVVAIASNIGGKQALETVQALLP
VLCQAHGLTPEQVVAIASNGGGKQALETVQRLLPVLCQAHGLTPEQVVAIASNIGGKQALETVQALL
PVLCQAHGLTPEQVVAIASHDGGKQALETVQALLPVLCQAHGLTPEQVVAIASHDGGKQALETVQAL
LPVLCQAHGLTPEQVVAIASNGGGKQALETVQRLLPVLCQAHGLTPQQVVAIASHDGGKQALETVQR
LLPVLCQAHGLTPQQVVAIASNGGGRPALESIVAQLSRPDPALAALTNDHLVALACLGGRPALDAVK
KGLG
ND5.3-DdCBE Right mitoTALE repeat
(SEQ ID NO: 165)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPQQVVAIASNIGGKQALETVQRLLPVLCQAHGLTPEQVVAIASNIGGKQALETVQRLLPVL
CQAHGLTPQQVVAIASNNGGKQALETVQRLLPVLCQAHGLTPEQVVAIASNIGGKQALETVQALLPV
LCQAHGLTPQQVVAIASNGGGKQALETVQRLLPVLCQAHGLTPQQVVAIASNGGGKQALETVQRLLP
VLCQAHGLTPEQVVAIASNIGGKQALETVQRLLPVLCQAHGLTPQQVVAIASNNGGKQALETVQRLL
PVLCQAHGLTPQQVVAIASNGGGKQALETVQRLLPVLCQAHGLTPEQVVAIASNIGGKQALETVQRL
LPVLCQAHGLTPEQVVAIASNGGGKQALETVQRLLPVLCQAHGLTPQQVVAIASNNGGKQALETVQR
LLPVLCQAHGLTPQQVVAIASNNGGKQALETVQRLLPVLCQAHGLTPQQVVAIASNGGGRPALESIV
AQLSRPDPALAALTNDHLVALACLGGRPALDAVKKGLG
ND5.3-DdCBE Left mitoTALE repeat
(SEQ ID NO: 167)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASHDGGKQALETVQALLPVLCQAHGLTPQQVVAIASNIGGKQALETVQRLLPVL
CQAHGLTPQQVVAIASNNGGKQALETVQRLLPVLCQAHGLTPEQVVAIASNGGGKQALETVQALLPV
LCQAHGLTPEQVVAIASNGGGKQALETVQALLPVLCQAHGLTPEQVVAIASHDGGKQALETVQALLP
VLCQAHGLTPEQVVAIASNGGGKQALETVQRLLPVLCQAHGLTPEQVVAIASNGGGKQALETVQALL
PVLCQAHGLTPEQVVAIASHDGGKQALETVQRLLPVLCQAHGLTPEQVVAIASNIGGKQALETVQAL
LPVLCQAHGLTPEQVVAIASNIGGKQALETVQALLPVLCQAHGLTPEQVVAIASNIGGKQALETVQAL
LPVLCQAHGLTPEQVVAIASNGGGKQALETVQRLLPVLCQAHGLTPQQVVAIASNIGGKQALETVQR
LLPVLCQAHGLTPQQVVAIASNGGGRPALESIVAQLSRPDPALAALTNDHLVALACLGGRPALDAVK
KGLG
ATP8-DdCBE Right mitoTALE repeat (Note: Terminal NG RVD recognizes
a mismatched T instead of a C in the reference genome)
(SEQ ID NO: 12)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPQQVVAIASNIGGKQALETVQRLLPVLCQAHGLTPEQVVAIASNGGGKQALETVQRLLPVL
CQAHGLTPQQVVAIASNNGGKQALETVQRLLPVLCQAHGLTPEQVVAIASNNGGKQALETVQALLPV
LCQAHGLTPQQVVAIASNNGGKQALETVQRLLPVLCQAHGLTPQQVVAIASHDGGKQALETVQRLLP
VLCQAHGLTPEQVVAIASNGGGKQALETVQRLLPVLCQAHGLTPQQVVAIASNGGGKQALETVQRLL
PVLCQAHGLTPQQVVAIASNGGGKQALETVQRLLPVLCQAHGLTPEQVVAIASNNGGKQALETVQRL
LPVLCQAHGLTPEQVVAIASNNGGKQALETVQRLLPVLCQAHGLTPQQVVAIASNGGGRPALESIVA
QLSRPDPALAALTNDHLVALACLGGRPALDAVKKGLG
ATP8-DdCBE Left mitoTALE repeat
(SEQ ID NO: 170)
DIADLRTLGYSQQQQEKIKPKVRSTVAQHHEALVGHGFTHAHIVALSQHPAALGTVAVKYQDMIAAL
PEATHEAIVGVGKQWSGARALEALLTVAGELRGPPLQLDTGQLLKIAKRGGVTAVEAVHAWRNALT
GAPLNLTPEQVVAIASNIGGKQALETVQALLPVLCQAHGLTPQQVVAIASNGGGKQALETVQRLLPVL
CQAHGLTPQQVVAIASNGGGKQALETVQRLLPVLCQAHGLTPEQVVAIASNIGGKQALETVQALLPV
LCQAHGLTPEQVVAIASNIGGKQALETVQALLPVLCQAHGLTPEQVVAIASNIGGKQALETVQALLPV
LCQAHGLTPEQVVAIASHDGGKQALETVQRLLPVLCQAHGLTPEQVVAIASNIGGKQALETVQALLP
VLCQAHGLTPEQVVAIASHDGGKQALETVQRLLPVLCQAHGLTPEQVVAIASNIGGKQALETVQALL
PVLCQAHGLTPEQVVAIASNIGGKQALETVQALLPVLCQAHGLTPEQVVAIASNIGGKQALETVQALL
PVLCQAHGLTPEQVVAIASHDGGKQALETVQRLLPVLCQAHGLTPQQVVAIASNGGGKQALETVQRL
LPVLCQAHGLTPEQVVAIASNIGGKQALETVQALLPVLCQAHGLTPQQVVAIASNGGGRPALESIVAQ
LSRPDPALAALTNDHLVALACLGGRPALDAVKKGLG

### EXAMPLES

### Example 1. Making Evolved DddA Mutants with Improved Editing Activity at TC Context

Phage-assisted non-continuous and continuous evolution (PANCE and PACE) was applied to evolve split-DddA (Thuronyi, B. W. et al. Nat Biotechnol 37, 1070-1079(2019)) domains (FIG. 2), resulting in variants that contained mutations in the N-terminal and C-terminal halves of split DddA (FIGS. 3A-3D). Exemplary (a) starter sequences and (b) resulting PACE-evolved variant DddA sequences are represented by SEQ ID NOs: 25-31.

These DddA mutations were cloned into the DdCBE architecture (MTS-right TALE-G1397 split DddA-(N/C)-UGI) and plasmid transfected into human HEK293T cells (FIG. 3A). At sites that were weakly editing by the wildtype DdCBE, editor variant T1380I improved editing efficiencies 1.2-2.3 fold (FIG. 3B), and variants Q1310R/S1330I/T1380I and T1380I/T1413I improved editing efficiencies by 4.8-4.9-fold up to 40%. (FIG. 3C). The indels associated with the highly active variants remained below the 0.1% detection limit (FIG. 3D). Using directed protein evolution, split G1397-DddA variants that show greatly improved editing efficiencies over the wildtype DddA were successfully evolved.

Additional PACE-evolved DddA halves are provided in FIG. 4, with corresponding sequences provided below. Each of the variants a PACE-evolved variant of DddA of SEQ ID NO: 25 (corresponding to residues 1290-1427 of canonical DddA):

Exemplary variant DddA fragments derived (e.g., using continuous evolution, such as PACE) from SEQ ID NO: 25 can include, for example SEQ ID NOs: 25, 32-54.

### Example 2. Continuous Evolution of CRISPR-Free Mitochondrial and Nuclear Base Editors with Enhanced Activity and Expanded Targeting Scope

Each human cell can contain hundreds of mitochondria and several hundred copies of circular mtDNA¹⁻³. The human mitochondrial genome contains tRNAs and rRNAs that enable mitochondrial translation of mtDNA genes encoding protein subunits of the electron transport chain. Due to the essential role of the mitochondria in energy homeostasis and other biological processes, single-nucleotide mutations in the mtDNA could contribute to developmental disorders, neuromuscular disease and cancer progression⁴⁻⁶. Whole genome analyses from large patient cohorts continue to reveal a growing number of mtDNA somatic substitutions that could contribute to human diseases⁷. To elucidate the role of these mutations in pathogenesis, there is an urgent need to develop technologies that enable the precise installation of point mutations within mtDNA. Such tools could also have the potential to correct deleterious mutations present in the mtDNA.

Traditional nuclease-based strategies to manipulate mtDNA make targeted double-strand breaks (DSBs) within mtDNA copies that contain specific mutations^{8,9}. Since DSBs in mtDNA lead to the destruction of that copy of mtDNA¹⁰⁻¹¹, this approach is useful for eliminating diseased copies of mtDNA¹². Nucleases, however, cannot introduce specific sequence changes in mtDNA. Genome editing agents including base editors^{13,14} and prime editors¹⁵ are capable of directly installing precise changes in a target DNA sequence, but typically rely on a guide RNA sequence to direct CRISPR-Cas proteins for binding to its target DNA. Due to the challenge of importing guide RNAs into the mitochondria, CRISPR-based systems have thus far not been reliably used for mtDNA engineering¹⁶

To begin to address this challenge, DdCBE was recently developed to enable targeted C⋅G-to-T⋅A conversions within mtDNA¹⁷. DdCBE uses two mitochondrial-localized TALE proteins to specify the double-stranded DNA (dsDNA) region for editing. Each TALE is fused to a non-toxic half of DddA cytidine deaminase and one copy of uracil glycosylase inhibitor protein to suppress uracil base excision repair. Binding of two TALE-split DddA-UGI fusions to adjacent sites promotes reassembly of functional DddA for deamination of target cytosines within the dsDNA spacing region. DdCBEs have since been applied for mitochondrial base editing in mice, zebrafish and plantsis¹⁸⁻²⁰.

In initial studies, a range of mtDNA editing efficiencies (4.6% to 49%) were observed depending on the position of the target C within the spacing region between the DNA-bound DdCBE halves¹⁷. It was hypothesized that enhancing the activity of split DddA could increase mtDNA editing efficiencies at putative 5′-TC contexts by improving the compatibility of DddA with different TALE designs and deaminase orientations.

Given the strict sequence preference of DddA, DdCBEs are currently limited predominantly to TC targets. As presented herein, an increase in DdCBE activity was sought at both TC and non-TC targets by applying rapid phage-assisted continuous evolution (PACE) and related non-continuous PANCE methods^{21,22}. Development of a selection circuit for DdCBE activity followed by PANCE and PACE resulted in several families of DddA variants with conserved mutations enriched during evolution. Evolved variants DddA6 and DddA11 mediated ˜4.3-fold average improvement in mtDNA base editing efficiency at TC targets compared to wild-type DddA. Importantly, DddA11 increased average bulk editing levels at AC and CC targets in the mtDNA and nucleus from <10% with canonical DdCBE to ˜15-40%. These variants collectively enable the installation or correction of C⋅G-to-T⋅A point mutations at both TC and non-TC targets, thus substantially expanding the overall utility of DdCBEs.

### Results

The all-protein cytosine base editor DdCBE uses TALE proteins and a double-stranded DNA-specific cytidine deaminase (DddA) to mediate targeted C⋅G-to-T⋅A editing. To improve editing efficiency and overcome the strict TC sequence-context constraint of DddA, the experiment used phage-assisted non-continuous and continuous evolution to evolve DddA variants with improved activity and expanded targeting scope. Compared to canonical DdCBEs, base editors with evolved DddA6 improved mitochondrial DNA (mtDNA) editing efficiencies at TC by 3.3-fold, on average. DdCBEs containing evolved DddA11 offered a broadened HC (H=A, C, or T) sequence compatibility for both mitochondrial and nuclear base editing, increasing average editing efficiencies at AC and CC targets from <10% for canonical DdCBE to 15-30%, and up to 50% in cell populations sorted to express both halves of DdCBE. We used these evolved DdCBEs to efficiently install disease-associated mtDNA mutations in human cells at non-TC target sites. DddA6 and DddA11 substantially increase the effectiveness and applicability of all-protein base editing.

### Adapting BE-PACE to Evolve TALE-Based DdCBEs

PACE uses an M13 phage that is modified to contain an evolving gene in place of gene III (gIII)²³. gIII encodes a capsid protein pIII that is essential for producing infectious phage progeny. To establish a selection circuit, gIII is encoded in an accessory plasmid (AP) within the E. coli host cell such that gIII expression is dependent on the evolving activity. Previously, a BE-PACE system to evolve CRISPR cytosine base editors was reported²¹. In this system, the AP encodes gIII under the control of a T7 promoter. A complementary plasmid (CP) encodes T7 RNA polymerase (T7 RNAP) fused to a degron through a 2-amino-acid linker (FIG. 19A). In the absence of C⋅G-to-T⋅A editing of the linker sequence, the degron triggers constitutive proteolysis of T7, preventing gIII expression (FIG. 19B). The target cytosines for DdCBE-mediated editing were defined as C₆ and C₇, where the subscripted numbers refer to their positions in the spacing region, counting the DNA nucleotide immediately after the binding site of the left-side TALE (TALE3) as position 1. Successful C⋅G-to-T⋅A editing of either one or both C₆ and C₇ targets introduces a stop codon within the linker to prevent translation of the degron tag with T7 RNAP. Active T7 RNAP then initiates gIII expression (FIG. 19B). The nucleotide at position 8 may be modified to either A, T, C or G without changing the protein sequence of T7 RNAP or the degron, thus enabling selection against TC and non-TC contexts (FIG. 19B).

It was previously observed that splitting DddA at G1397 resulted in higher editing efficiencies of target Cs positioned within the transcription template strand compared to splitting DddA at G1333¹⁷. Given that C₆ and C₇ targets were located in the transcription template strand, a DdCBE that consisted of a left-side TALE (TALE3) and a right-side TALE (TALE4) flanking a 15-bp spacing region, with the target C₇ positioned 7-bp from the 3′ end of the transcription template strand was designed (FIG. 19C). Since E. coli lack mitochondria and the selection circuit relies on editing plasmid DNA in the cytoplasm, the mitochondria-targeting signal sequences were removed. One copy of UGI was also fused to the N-terminus of the TALE protein, which was previously shown to result in higher editing of nuclear DNA compared to C-terminal UGI fusions¹⁷ (FIG. 19C). Given that DddA has virtually no activity against non-TC sequences, it was hypothesized that a DdCBE architecture with maximal editing efficiency would provide a favorable starting point for subsequent evolution for activity against non-TC contexts. UGI-TALE3-DddA-G1397-N and UGI-TALE4-DddA-G1397-C, the DdCBE referred to hereafter as T7-DdCBE, were encoded in the selection phage (SP) to co-evolve both halves of DdCBE (FIG. 19A). The phage genome is continuously mutagenized by an arabinose-inducible mutagenesis plasmid (MP6)²⁴ (FIG. 19A).

To modulate selection stringency, host strains 1 to 4 were generated. Each host strain contained combinations of AP and CP with different ribosome binding site strengths, such that strain 1 resulted in the lowest selection stringency and strain 4 provided the highest stringency. All tested CPs encoded the TCC linker sequence (FIG. 23A). Then, propagation of the SP in these host strains was tested overnight. At the highest stringency, ˜100-fold overnight phage propagation of an SP containing an active T7-DdCBE was observed, consistent with DdCBE's ability to edit 5′-TC targets. Importantly, phage containing an inactivating E1347A mutation within DddA of T7-DdCBE (dead T7-DdCBE phage) did not propagate (FIG. 23B). These results establish the dependence of phage propagation on DdCBE activity, and that BE-PACE can be successfully adapted to select TALE-based DdCBEs.

### Phage-Assisted Evolution of DdCBE Towards Higher Editing Efficiency at 5′-TC

It was reasoned that beginning evolution with PANCE may be useful to increase activity and phage propagation before moving into PACE²². PANCE is less stringent because fresh host cells are manually infected with SP from a preceding passage, so no phage is lost to continuous dilution.

To evolve DdCBEs for higher C⋅G-to-T⋅A editing activity at TC targets, PANCE of canonical T7-DdCBE was initiated by infecting SP into high-stringency strain 4 transformed with MP6 (FIG. 23A). After seven passages, phage populations from all four replicates propagated approximately 10,000-fold overnight (FIG. 23C). Isolated clonal phage from two or more independent replicates were enriched for the mutations T1372I, M1379I, and T1380I within the DddA gene (FIG. 36).

To validate the editing activity associated with these DddA genotypes, we incorporated each of these mutations into our previously published G1397-split DdCBEs that targeted human MT-ATP8, MT-ND4 and MT-ND5 (FIGS. 37A-37B)¹⁷. HEK293T cells were plasmid-transfected with canonical versions of ATP8-DdCBE, ND4-DdCBE and ND5.2-DdCBE and compared their editing efficiencies to those produced from the corresponding DdCBEs containing the DddA mutants. While T1372I and M1379I impaired editing, T1380I increased C⋅G-to-T⋅A conversions by an average of 1.2- to 2.0-fold across the three mtDNA sites (FIG. 23D). It is possible that the benefit of T1372I and M1379I may require additional mutations evolved during PANCE but not tested in mammalian cells. These results indicate that PANCE of canonical T7-DdCBE was able to yield a DddA variant that resulted in modest improvements in TC editing. The DddA (T1380I) mutant is referred to as DddA1 hereinafter (FIG. 20A).

To further increase selection stringency, PACE was conducted using an SP encoding the DddA1 variant of T7-DdCBE (T7-DdCBE-DddA1). After 140 h of continuous propagation at a flow rate of 1.5 to 3.0 lagoon vol/h, enrichment of mutations that were distinct across the four replicates was observed, with the T1380I mutation maintained across all lagoons (FIG. 38). The most enriched genotype was selected in each of the four replicates (DddA2, DddA3, DddA4 and DddA5) and tested their mtDNA editing efficiencies (FIGS. 20A-20B). DddA2, DddA3, DddA4 and DddA5 improved average editing efficiencies at target TCs within MT-ND5 and MT-ATP8 from 7.6±2.4% with starting DddA to 14±5.8%, 22±6.1%, 21±7.9% and 24±4.4%, respectively (FIGS. 20C-20D).

The T1413I mutation in DddA4, which is in the C-terminal half of split-DddA, improved base editing activity of DddA4 by an average of 1.6-fold compared to DddA1. Given that T1413I is positioned along the interface between the two split DddA halves (FIG. 20B), it was hypothesized that this mutation could be promoting the reconstitution of split DddA halves. Incorporating T1413I into DddA5 to form DddA6 (Q1310R+S1330I+T1380I+T1413I) resulted in a modest editing improvement to 26±3.7%, representing a 3.4-fold average improvement in TC editing activity compared to wild-type DddA (FIGS. 20C-20D). Close to 90% of the edited alleles produced from DddA6 contained a TCC-to-TTT conversion, suggesting that consecutive cytosines are likely targets for processive base editing (FIGS. 20E-20F). These results establish DddA6 as a dsDNA cytidine deaminase variant with enhanced editing activity at TC sequences.

DddA6 was evolved from T7-DdCBE containing DddA split at G1397. To check if DddA6 is compatible with the G1333 split, DddA6 was tested at three mtDNA sites using DdCBEs split at G1333 and observed a 1.3- to 3.6-fold improvement in editing efficiencies compared to wild-type DddA (FIGS. 24A-24C). These data indicate that mutations in DddA6 can enhance mtDNA editing efficiencies of the G1333 split variant, but the extent of this improvement is lower than with the G1397 split. It was noted that increases in editing efficiencies with DddA6 compared to wild-type DddA were modest at sites that exhibit efficient editing even with wild-type DddA, such as MT-ND1 and MT-ND4 (FIGS. 24A-24D). For such sites already efficiently edited with canonical DdCBEs, other deaminase-independent factors, such as mtDNA repair, could limit editing efficiency more than deaminase activity.

Evolving DddA Variants with Expanded Sequence Context Compatibility

Next, the enhanced activity of DddA6 was assessed to determine if it would enable base editing at target cytosines not in the native TC sequence context. To measure the activity of DddA6 and subsequent evolved variants at TC and non-TC targets, a bacterial plasmid assay was designed to measure C⋅G-to-T⋅A conversion of a target C within an NC₇N context, where N=A, T, C or G. A plasmid-encoded NC₇N target library was transformed into bacteria expressing T7-DdCBE that contained a given DddA variant. After overnight incubation, the plasmid library was isolated and subjected to high-throughput sequencing to measure the C⋅G-to-T⋅A conversion at each of the 16 NC₇N targets (FIG. 21A).

Consistent with earlier human mtDNA editing results, DddA6 improved the average editing efficiencies of bacterial plasmids containing TC₇N substrates by approximately 1.3-fold. DddA6-mediated editing at non-TC sequences, however, remained negligible (<0.20%) (FIG. 21B), suggesting the possibility of further evolving DddA to deaminate non-TC targets.

The linker sequence was modified in the CP to contain ACC, CCC or GCC. Next, three high-stringency E. coli hosts (strains 5, 6, and 7) were generated by co-transforming cells with APi and one of three possible CP plasmids (CP2-ACC, CP2-CCC or CP2-GCC) (FIG. 19B and FIG. 25A). The host strains were infected with SP encoding T7-DdCBE-DddA1. A large drop in overnight phage titers across strains 5, 6, and 7 suggested that the starting T7-DdCBE-DddA1 activity against non-TC sequences was too low to support PACE, so evolution was initiated with PANCE (FIG. 25B). A round of mutagenic drift was first conducted to diversify the phage genome in the absence of selection pressure²⁵. Next, three parallel PANCE campaigns of T7-DdCBE-DddA1 (PANCE-ACC, PANCE-CCC and PANCE-GCC) were initiated. Each campaign was challenged with a non-TC linker and was conducted in four replicates (FIGS. 25C-25E).

Phage that propagated >10,000-fold overnight after nine passages of PANCE was isolated. The DddA genotypes surviving PANCE were strongly enriched for N1342S and E1370K mutations across all PANCE campaigns. Positions A1341 and G1344 were hotspots for substitutions to different amino acids depending on the target linker sequence (FIG. 39).

Given the substantial increase in phage propagation strength after nine PANCE passages (surviving in total ˜10¹⁶⁻¹⁹-fold dilution), selection stringency was increased by challenging three surviving phage populations (PANCE-CCC-B, PANCE-GCC-A and PANCE-GCC-D) to 138 hours of PACE at a flow rate of 1.5 to 3.5 lagoon vol/h. For PACE, the same MP6-transformed strains 6 and 7 were used that had been applied in earlier PANCE campaigns (FIG. 25A). The resulting PACE-evolved DddA variants converged on the additional mutations T1314A, E1396K, and T1413I (FIG. 40). Given that earlier PACE variant DddA4, which contained T1380I and T1413I mutations, showed ˜1.6-fold improved TC editing relative to DddA1, T1413I could be broadly beneficial for enhancing DddA activity at both TC and non-TC contexts.

### Characterizing Sequence Context Preferences of DddA Variants

From the phage populations that survived PACE against a CCC- or GCC linker target, six to eight clones and isolated five DddA variants (DddA7, DddA8, DddA9, DddA10 and DddA11) were sequenced based on the consensus mutations within DddA (FIG. 21C). Then, their sequence context preferences were profiled using the same bacterial NC₇N plasmid assay used to characterize DddA6 (FIG. 21A).

All variants, except DddA8, maintained or improved editing efficiencies at TC, averaging 22-50% (FIG. 21B). DddA9 and DddA10 resulted in approximately 2.0-fold higher TC editing than canonical T7-DdCBE but very low CC editing (<3.0%) (FIG. 21B). While the average AC and CC editing levels by canonical T7-DdCBE were negligible (<0.66%), DddA7, DddA8 and DddA11 yielded an average of 3.4-5.1% editing at these contexts within bacterial plasmids (FIG. 21B). These results demonstrate that PACE can be successfully applied to evolve for DddA variants that show expanded targeting activity beyond TC.

To validate the activity of these DddA variants in human mtDNA, we replaced wild-type DddA in ND5.2-DdCBE and ATP8-DdCBE with DddA7, DddA8, Ddd9, DddA10, or DddA11 (FIGS. 37A-37B). Consistent with bacterial plasmid editing results, DddA9 and DddA10 resulted in similar improvements in TC editing as DddA6, but did not exhibit consistent non-TC editing across multiple mtDNA sites (FIGS. 21D-21E).

Among variants DddA7, DddA8 and DddA11, DddA11 supported the highest mtDNA editing efficiencies at TC (18-25%), AC (4.3-5.0%), and CC (7.6-16%) (FIGS. 21D-21E). Processive editing of consecutive cytosines in the spacing region could edit a preceding cytosine to a thymine, thus changing the starting ACC target into ATC₁₀ in MT-ND5 and ATC₉ in MT-ATP8 (FIGS. 21D-21E). To clarify if C₁₀ and C₉ are edited as ACC or ATC targets, the percentage of edited alleles that contained an ACT or ATT product were compared. It was noted that majority of the edited alleles retained the preceding 5′-C(48% for ND5.2-DdCBE and 27% for ATP8-DdCBE) (FIGS. 26A-26B). Thus, C₁₀-to-T₁₀ and C₉-to-T₉ conversions mediated by ND5.2-DdCBE and ATP8-DdCBE, respectively, were classified as editing of CC sequences (FIGS. 21D-21E).

Given that Ddd8 and DddA11 resulted in higher AC and CC editing than DddA7 in human mtDNA, it was hypothesized that DddA8 and DddA11 might be more toxic than DddA7 when expressed in bacteria. This could explain the lower editing activity of these variants relative to DddA7 in the context profiling assay performed in bacteria (FIG. 21B).

Next, DddA6 and DddA11 were tested in three other human cell lines for mitochondrial base editing using ND5.2-DdCBE. The right and left halves of ND5.2-DdCBE were fused to fluorescent markers eGFP and mCherry, respectively, by a self-cleaving P2A sequence²⁶ to enable fluorescence-activated cell sorting of nucleofected cells that express both halves of the DdCBE. For poorly transfected cells such as HeLa, enriching cells expressing both eGFP and mCherry (eGFP⁺/mCherry⁺) substantially boosted TC and non-TC editing levels from <1% to 4-31% (FIGS. 27A and FIGS. 41A-41C). In K562 cells and U2OS cells, this enrichment strategy improved average editing by 11-fold and 1.5-fold, respectively, to efficiencies ranging from 20-60% (FIGS. 27B-27C). These results indicate that cell lines other than HEK293T support improved mitochondrial base editing by evolved DddA variants.

Given that DddA11 resulted in the highest mtDNA editing efficiencies at AC, CC, and TC targets, a reversion analysis was conducted on DddA11 to identify the individual contributions of the mutations. Eight different reversion mutants of DddA11 (11a-h) were generated. Compared to DddA11a, DddA11e had detectable AC and CC editing at MT-ATP8 (0.48-0.76%), indicating that acquisition of N1342S alone was sufficient for modest editing activity at non-TC sequences (FIG. 28). The additive effect of N1342S and E1370K in 11 g further increased AC and CC editing efficiencies, up to 3.4-5.4%. S13330I and A1341V exerted their positive epistatic effect only in the presence of N1342S and E1370K (compare 11b to 11) (FIG. 28). These results collectively suggest that the combination of the six mutations S1330I, A1341V, N1342S, E1370K, T1380I and T1413I enable DddA11 to catalyze efficient base editing at broadened AC, CC, and TC contexts that are poorly edited by canonical DdCBE.

To determine if improved activity of the evolved DddA6 and DddA11 variants might influence the editing window for DdCBE editing, a separate library of 14 target plasmids was generated for editing by canonical and evolved T7-DdCBEs containing the G1397 split, using the bacterial plasmid assay for context profiling. The spacing region in each target plasmid contained repeats of TC sequences ranging from 12-18-bp. These repeats were positioned on the top or bottom DNA strand (FIG. 29A).

High-throughput sequencing of the target plasmids revealed the highest editing efficiencies following treatment with DddA11 (FIGS. 29B-29H). Within 12 to 15-bp spacing regions, the canonical and evolved DdCBEs preferentially edited cytosines positioned 4-6 nucleotides and 6-8 nucleotides upstream of the 3′ end of the bottom strand and top strand, respectively (FIG. 21F and FIGS. 29B-29E). At 16 to 18-bp spacing regions, canonical and DddA6-containing DdCBEs maintained modest editing of target cytosine positioned 6 nucleotides upstream of the 3′ end of the bottom strand, but failed to efficiently edit cytosines in the top strand (FIG. 21F and FIGS. 29F-29H). In contrast, DddA11 retained activity for top-strand cytosines positioned 7-9 nucleotides upstream of the 3′ end, but efficiencies were substantially lower compared to shorter spacing lengths (FIG. 21F, compare FIGS. 29B-29E to FIGS. 29F-29H). These results indicate that the editing windows of evolved variants split at G1397 were generally similar to that of canonical DdCBE, with DddA11 exhibiting a larger editing window for spacing lengths >15-bp.

### Evolved DdCBE-Mediated Editing of Nuclear DNA

It was previously demonstrated that nuclear-localized DdCBE containing two copies of UGI fused to the N-terminus can mediate base editing at nuclear TC targets, which may provide useful alternatives to CRISPR CBEs when guide RNA or PAM requirements are limiting¹⁷ (FIGS. 42A-42B). To test if DddA11 also expands the targeting scope of nuclear DNA base editing, HEK293T cells were transfected with DdCBEs that targeted nuclear SIRT6 or JAK2 loci²⁷. When localized to the nucleus in the G1397-split orientation, DddA11 substantially improved AC, CC and GC nuclear base editing from a typical range of 0-14% to 17-35% (FIGS. 21G-21H; see FIGS. 26C-26D for frequencies of edited alleles). These results collectively show that DddA11 substantially enhances non-TC editing efficiencies for all-protein base editing of both mitochondrial and nuclear DNA.

To assess for potential nuclear off-target editing, the off-target prediction tool PROGNOS²⁸ was used to rank human nuclear DNA sequences that were predicted to be targeted by the TALE repeats in SIRT6- and JAK2-DdCBE. HEK293T cells were treated with the canonical or evolved DdCBEs and performed amplicon sequencing of the top 9-10 predicted off-target sites for each base editor (Table 9). The average frequencies in which C⋅G base pairs within the predicted off-target spacing region were converted to T⋅A base pairs were very similar between the canonical and evolved DdCBEs (FIGS. 30A-30B). These results suggest that DddA6 and DddA11 did not increase nuclear off-target editing within a subset of computationally predicted off-target sites for a given pair of TALE repeats.

### Attempts to Further Increase Activity at GC Sequences

It was noted that DddA11 was active mostly at GC₇C₆ and not GC₇C₆(FIG. 21B). Given that a single C₆-to-T₆ conversion in a CC context is sufficient to generate a stop codon (FIG. 19B), the selection pressure to evolve acceptance of GC substrates was likely attenuated. To increase selection stringency, the linker was modified to encode either GCA or GCG such that only DddA variants that show activity at GC were able to restore active T7 RNAP (FIG. 31A). To test for overnight phage propagation when challenged with these modified linkers, host strains 9 and 10 were generated. Strain 9 contains a CP encoding the GCA linker and strain 10 contains a CP encoding the GCG linker (FIG. 31B). Consistent with the weak GC activity of DddA11 (FIG. 21B), a drop in overnight phage titers in strains 9 and 10 was observed, suggesting that the activity of T7-DdCBE-DddA11 against GCA and GCG was too low to support PACE (FIG. 31C and Supplementary Discussion, below).

PANCE of T7-DdCBE-DddA11 was initiated in MP6-transformed host strains 9 and 10. After 12 passages, overnight phage propagation increased to approximately 100- to 1,000-fold (FIG. 31D and Supplementary Discussion, below). The surviving phage isolates from round 9 and round 12 were sequenced to derive four consensus DddA genotypes (FIG. 32A and FIG. 43). When tested as DdCBEs targeting four mtDNA sites, the evolved variants did not show consistently improved editing efficiencies or targeting scope compared with DddA11, although it was noted that variant 7.9.1 showed higher editing efficiencies at TC targets compared to DddA6 and DddA11 (FIGS. 32B-32E and Supplementary Discussion, below). These results suggest that variants of DddA11 that are able to process GC substrates with improved efficiency are very rare.

### Mitochondrial Off-Target Activity of Evolved DddA Variants

To profile off-target editing activities of DdCBEs containing DddA6 and DddA11, ATAC-seq was performed of whole mitochondrial genomes from HEK293T cells transfected with plasmids encoding canonical or evolved variants of ND5.2-DdCBE or ATP8-DdCBE. A sequencing depth of approximately 3,000-8,000× was obtained per sample.

Consistent with previous results¹⁷, the average frequencies of mtDNA-wide off-target editing arising from canonical DdCBEs (0.033±0.002%) were comparable to the untreated control or DdCBEs containing dead DddA6 (0.028±0.001%) (FIG. 21I). Off-target frequencies associated with ND5.2-DdCBE were 1.5-fold higher for DddA6 and DddA11 compared to wild-type DddA, and 3.0- to 4.8-fold for ATP8-DdCBE (FIG. 21I). The average frequencies of all unique single-nucleotide variants (SNVs) containing a C⋅G-to-T⋅A conversion in cells treated with ATP8-DdCBE or ND5.2-DdCBE variants was analyzed (FIGS. 33A-33F). While all 28 SNVs in cells treated with canonical ATP8-DdCBE were <1.0% (FIG. 33A), 76 and 159 SNVs with >1% editing were detected for cells treated with DddA6 and DddA11, respectively (FIGS. 33B-33C). As expected, these results suggest that deaminase-dependent off-target editing increases in the presence of DddA variants with higher activity and expanded targeting scope.

In addition to deaminase-dependent off-target editing, the TALE repeats also contribute to overall off-target activity. For ND5.2-DdCBEs containing DddA6 or DddA11, fewer than 4 SNVs with >1% frequency were observed—far lower than those observed in ATP8-DdCBE containing the same DddA6 or DddA11 (compare FIGS. 33B-33C to FIGS. 33E-33F). It is hypothesized that TALE repeats that bind promiscuously to multiple DNA bases are more likely to result in higher off-target editing when fused to the evolved DddA variants^{29,30}.

These results collectively indicate that mtDNA off-target editing increases slightly for DdCBEs that use DddA6 and DddA11, as expected given their higher activity and widened targeting scope, but that the frequency of off-target editing per on-target editing event remains similar to that of canonical DdCBE (FIG. 33G).

Installing Previously Inaccessible Pathogenic Mutations in mtDNA

To demonstrate the utility of evolved DddA variants with broadened sequence context compatibility, three DdCBEs were designed to install disease-associated C⋅G-to-T⋅A mutations at non-TC positions in human mtDNA. ND4.2-DdCBE installs the m.11696G>A mutation in an ACT context. This mutation is associated with Leber's hereditary optic neuropathy (LHON)³¹. ND4.3-DdCBE installs the m.11642G>A mutation in a GCT context and ND5.4-DdCBE installs the m.13297G>A mutation in a CCA context. Both of these mutations were previously implicated in renal oncocytoma³². These three mutations occur in coding mitochondrial genes and result in either a premature stop codon or a change in amino acid sequence (FIG. 22A).

The editing efficiencies among DdCBEs containing wild-type DddA, DddA6 or DddA11 split at G1397 were compared. While canonical DdCBEs resulted in negligible editing at non-TC sites, DddA11 edited the on-target cytosines at average efficiencies ranging from 7.1% to 29% in bulk HEK293T cell populations (FIGS. 22B-22D). Despite the very low levels of GC editing mediated by DddA11 in the bacteria plasmid assay (FIG. 21B), DddA11 yielded 7.1±0.69% on-target GC₆ editing when tested in ND4.3-DdCBE (FIG. 22B). The majority of the alleles containing the on-target edit, however, also harbored bystander edits that would result in unintended changes to the protein coding sequence (FIG. 34A).

Unlike ND4.3-DdCBE, ND4.2- and ND5.4-DdCBE resulted in higher bulk editing efficiencies ranging from 17-29% (FIGS. 22C-22D). More than 57% of the edited alleles contained the desired C⋅G-to-T⋅A on-target edit and a silent bystander edit (FIGS. 34B-34C). DddA11 tested in the G1333 orientation resulted in lower on-target editing compared to the G1397 orientation (FIG. 22C). No on-target editing was detected with DddA11 when the target cytosine falls outside the preferred editing window of the G1333 split¹⁷ (FIG. 22D). These results collectively indicate that DddA11 tested in the G1397 split orientation enables much higher levels of base editing at AC, CC and GC targets than wild-type DddA, even though absolute editing levels at GC targets are modest.

Next, the phenotypic consequences of installing the missense m.11696G>A mutation and the nonsense m.13297G>A mutation were assessed in human mtDNA. eGFP⁺/mCherry⁺ cells were sorted for to enrich those that expressed both halves of DdCBE. This enrichment increased average on-target editing from 17-29% in unsorted cells to 35-43% in sorted cells (FIGS. 22C-22D). Compared to sorted cells containing dead DddA, sorted cells treated with DddA11-containing ND4.2- and ND5.4-DdCBEs exhibited reduced rates of basal and uncoupled respiration (FIG. 22E and FIG. 22G). These results establish that DddA11 can install candidate pathogenic mutations that canonical DdCBEs are unable to access, and that these edits can occur at levels sufficient to result in altered mitochondrial function. These capabilities could broaden disease-modelling efforts using mitochondrial base editing.

### Discussion

As recently reported, DdCBEs enable installation of precise mutations within mtDNA for the first time, but target cytosines are primarily limited to 5′-TC contexts, and some target sites are edited with low efficiencies (≤5%)¹⁷. To address these challenges, PACE was applied to rapidly evolve DdCBEs towards improved activity and expanded targeting scope. This work resulted in two DddA variants, DddA6 and DddA11, that function in DdCBEs to mediate mitochondrial and nuclear base editing. These variants improve editing activity both at TC sequences and at previously inaccessible non-TC targets, while maintaining comparably high on-target to off-target editing ratios.

To edit target cytosines in TC contexts, starting with canonical DdCBE is recommended. If editing efficiency is low, DddA6 can be used to increase editing at TC while minimizing bystander editing of any cytosines in the editing window that are not preceded by a T.

For target cytosines in non-TC contexts, DddA11 enables editing of AC and CC targets much more efficiently than canonical DdCBE. DddA11 typically supports higher C⋅G-to-T⋅A conversion in the G1397 split orientation compared to the G1333 split orientation. It is possible that initiating PACE with an SP encoding a G1333 split may result in mutations distinct from those found in DddA11. In addition, encoding the UGI on the C-terminus of T7-DdCBE during PACE may enrich additional mutations that favor the reassembly of mitochondrial DdCBEs containing this architecture.

Given that DddA11 is active at TC, AC and CC contexts, bystander editing is more likely with this variant than with canonical DdCBE. To minimize bystander editing, users may design different TALE binding sites that reduces the number of non-target cytosines within the editing window (FIG. 21F and FIGS. 29A-29H).

Additional protein evolution or engineering could further improve the editing efficiency of DddA variants, especially at GC targets. While the structure of DddA bound to its dsDNA target is currently unavailable, structural alignment of DddA to existing deaminases with altered sequence specificities could offer insights for further DddA engineering efforts (FIGS. 35A-35B and Supplementary Discussion).

### Supplementary Discussion

### Further Evolution of T7-DdCBE-DddA11 for Improved GC Activity

Previously, a PANCE of canonical T7-DdCBE was performed using a strains 9 and 10 transformed with MP6. These strains expressed the GCA or GCG linker, respectively (FIG. 31A). After fifteen passages, the overnight fold phage propagation increased to 103, representing >1,000-fold improvement. Clonal sequencing of phage isolated at the end of PANCE, however, revealed a stochastic frameshift mutation within the open reading frame encoding either the left- or right-half of T7-DdCBE (data not shown). It was thought that the premature stop codons helped improve phage fitness by reducing the translational burden on the phage, thus increasing phage propagation in a manner that was independent of the deamination activity of DddA. Given that DddA11 already exhibited a broadened targeting scope and non-zero GC activity (FIG. 22B), it was hypothesized that DddA11 could be a promising evolutionary stepping-stone to serve as a starting target for evolving DddA variants towards higher GC activity.

PANCE of T7-DdCBE containing DddA11 in duplicates was initiated using the same MP6-transformed strains 9 and 10. One replicate in PANCE-GCA and one replicate PANCE-GCG evolved ‘cheaters’ in which gIII was recombined into the phage genome. The PANCE schedules shown in FIG. 31D are for the other replicates that do not contain gIII within the SP genome. Six to eight plaques were isolated from each replicate after round 9 and round 12 for clonal sequencing. The mutation N1378S was strongly enriched in PANCE-GCA and PANCE-GCG. PANCE-GCA also showed strong consensus for the additional mutations A1341I and P1394S (FIG. 43).

Two strongly enriched genotypes (7.9.1 and 7.12.1) and two moderately enriched genotypes (7.12.2 and 7.12.3) were selected for validation of mtDNA base editing activity in human cells (FIG. 32A). Variant 7.12.1 generally resulted in a 2.2- to 27-fold decline in AC and CC editing compared to DddA11 (FIGS. 32B-32E). Variants 7.9.1 (SEQ ID NO: 55), 7.12.2 (SEQ ID NO: 57), and 7.12.3 (SEQ ID NO: 58) improved TC and non-TC editing by 1.4- to 1.6-fold when tested as ND4.3-DdCBE, but did not enhance GC editing compared to DddA11 (FIG. 32B). ND5.4-DdCBE containing variant 7.9.1 resulted in comparable editing to DddA11 at AC and CC targets (FIG. 32C). When tested at other sites, variants 7.9.1, 7.12.2, and 7.12.3 improved TC editing by an average of 1.2-fold compared to DddA11. These variants, however, generally resulted in lower non-TC compared to DddA11 when tested as ND5.2-DdCBE and ATP8-DdCBE (FIGS. 32D-32E).

### Structure Alignment of DddA to APOBEC3G

Previously, ssDNA-specific APOBEC3G cytidine deaminase was identified, which has an intrinsic 5′-CC preference¹, as the closest structural relative to DddA. The catalytic domain of human APOBEC3G complexed with its ssDNA 5′-CCA substrate² was aligned with DNA-free DddA. The PACE-derived DddA variants DddA8 and DddA11 expanded the putative TC sequence preference to include AC and CC (FIG. 21B). These variants contained mutations A1341V, N1342S, G1344R and G1344S that are positioned within a loop that aligns most closely to loop 3 of APOBEC3G (FIGS. 35A-35B). Previous studies identified DNA-binding loop 3 to be critical for enhancing the catalytic activity of APOBEC3G at 5′CC³. In this Example, the N1342S nucleotide substitution in DddA11e increased TC editing by 1.3-fold and yielded low but detectable AC and CC editing (FIG. 28). These results suggest that the DddA loop containing N1342 could be engineered to improve the catalytic activity of DddA and support deamination at non-TC contexts.

In APOBEC3G, residue D317 in loop 7 is critical for selectivity towards C₋₁³ (FIG. 35B). Context-specific PANCE of DddA strongly enriched for E1370K across all tested linkers of ACC, CCC and GCC (FIG. 39). Given that loop 7 of APOBEC3G spatially aligns with the DddA loop containing E1370K, E1370K could also be involved in altering the substrate selectivity of DddA (FIG. 35B).

### Methods

### General Methods and Molecular Cloning.

Antibiotics (Gold Biotechnology) were used at the following working concentrations: carbenicillin 100 μg/mL, spectinomycin 50 μg/mL, chloramphenicol 25 μg/mL, kanamycin 50 g/mL, tetracycline 10 μg/mL, streptomycin 50 μg/mL. Nuclease-free water (Qiagen) was used for PCR reactions and cloning. For all other experiments, water was purified using a MilliQ purification system (Millipore). PCR was performed using Phusion U Green Multiplex PCR Master Mix (ThermoFisher Scientific), Phusion U Green Hot Start DNA Polymerase (ThermoFisher Scientific) or Phusion Hot Start II DNA polymerase (ThermoFisher Scientific). All plasmids were constructed using USER cloning (New England Biolabs) and cloned into Machi chemically competent E. coli cells (ThermoFisher Scientific). Unless otherwise noted, plasmid or SP DNA was amplified using the Illustra Templiphi 100 Amplification Kit (GE Healthcare Life Sciences) prior to Sanger sequencing. Plasmids for bacterial transformation were purified using Qiagen Miniprep Kit according to manufacturer's instructions. Plasmids for mammalian transfection were purified using Qiagen Midiprep Kit according to manufacturer's instructions, but with 1.5 mL of RNAse A (1,000 μg/mL) added to Resuspension buffer. Codon-optimized sequences for human cell expression were obtained from GenScript. The amino acid sequences of all DdCBEs and DddA variants are provided in Sequences, below. A full list of bacterial plasmids used in this work is given in Table 4.

TABLE 4
List of bacterial plasmids used in this Example

| | | | | | ORF2 | |
|---|---|---|---|---|---|---|
| | | | ORF1 | | | [RBS] |
| Name | Class (res) | Origin | Promoter | [RBS] Genes | Promoter | Genes |
| pJC175e- | AP (carb^{R}) | SC101 | Pₚₛₚ | [SD8] gIII | P_{ProD} | [SD8] |
| DddI | | | | | | DddI-VSV- |
| | | | | | | G |
| MP6 | MP (chlor^{R}) | cloDF13 | P_{BAD} | dnaQ926, dam, seqA, | P_{c} | araC |
| | | | | emrR, ugi, cda1 | | |
| AP1 | AP (carb^{R}) | SC101 | P_{T7} | [SD8] gIII | | |
| AP2 | AP (carb^{R}) | SC101 | P_{T7} | [sd8] gIII | | |
| CP1-TCC | CP (spec^{R}) | ColE1 | P_{Pro1} | [sd2] T7 RNAP-TCC | | |
| | | | | linker-degron | | |
| CP2-TCC | CP (spec^{R}) | ColE1 | P_{Pro1} | [sd4U] T7 RNAP-TCC | | |
| | | | | linker-degron | | |
| CP2-ACC | CP (spec^{R}) | ColE1 | P_{Pro1} | [sd4U] T7 RNAP-ACC | | |
| | | | | linker-degron | | |
| CP2-CCC | CP (spec^{R}) | ColE1 | P_{Pro1} | [sd4U] T7 RNAP-CCC | | |
| | | | | linker-degron | | |
| CP2-GCC | CP (spec^{R}) | ColE1 | P_{Pro1} | [sd4U] T7 RNAP-GCC | | |
| | | | | linker-degron | | |
| CP3-GCA | CP (spec^{R}) | ColE1 | P_{Pro1} | [sd5] T7 RNAP-GCA | | |
| | | | | linker-degron | | |
| CP3-GCG | CP (spec^{R}) | ColE1 | P_{Pro1} | [sd5] T7 RNAP-GCG | | |
| | | | | linker-degron | | |
| pBM10a | NCN target (carb^{R}) | SC101 | N.A. | ACA target | | |
| pBM10b | NCN target (carb^{R}) | SC101 | N.A. | ACT target | | |
| pBM10c | NCN target (carb^{R}) | SC101 | N.A. | ACC target | | |
| pBM10d | NCN target (carb^{R}) | SC101 | N.A. | ACG target | | |
| pBM10e | NCN target (carb^{R}) | SC101 | N.A. | TCA target | | |
| pBM10f | NCN target (carb^{R}) | SC101 | N.A. | TCT target | | |
| pBM10g | NCN target (carb^{R}) | SC101 | N.A. | TCC target | | |
| pBM10h | NCN target (carb^{R}) | SC101 | N.A. | TCG target | | |
| pBM10i | NCN target (carb^{R}) | SC101 | N.A. | CCA target | | |
| pBM10j | NCN target (carb^{R}) | SC101 | N.A. | CCT target | | |
| pBM10k | NCN target (carb^{R}) | SC101 | N.A. | CCC target | | |
| pBM101 | NCN target (carb^{R}) | SC101 | N.A. | CCG target | | |
| pBM10m | NCN target (carb^{R}) | SC101 | N.A. | GCA target | | |
| pBM10n | NCN target (carb^{R}) | SC101 | N.A. | GCT target | | |
| pBM10o | NCN target (carb^{R}) | SC101 | N.A. | GCC target | | |
| pBM10p | NCN target (carb^{R}) | SC101 | N.A. | GCG target | | |
| pBM22a | Window profiling | SC101 | N.A. | (TC)₁₂ target, top strand | | |
| | (carb^{R}) | | | | | |
| pBM22b | Window profiling | SC101 | N.A. | (TC)₁₃ target, top strand | | |
| | (carb^{R}) | | | | | |
| pBM22c | Window profiling | SC101 | N.A. | (TC)₁₄ target, top strand | | |
| | (carb^{R}) | | | | | |
| pBM22d | Window profiling | SC101 | N.A. | (TC)₁₅ target, top strand | | |
| | (carb^{R}) | | | | | |
| pBM22e | Window profiling | SC101 | N.A. | (TC)₁₆ target, top strand | | |
| | (carb^{R}) | | | | | |
| pBM22f | Window profiling | SC101 | N.A. | (TC)₁₇ target, top strand | | |
| | (carb^{R}) | | | | | |
| pBM22g | Window profiling | SC101 | N.A. | (TC)₁₈ target, top strand | | |
| | (carb^{R}) | | | | | |
| pBM23a | Window profiling | SC101 | N.A. | (TC)₁₂ target, bottom | | |
| | (carb^{R}) | | | strand | | |
| pBM23b | Window profiling | SC101 | N.A. | (TC)₁₃ target, bottom | | |
| | (carb^{R}) | | | strand | | |
| pBM23c | Window profiling | SC101 | N.A. | (TC)₁₄ target, bottom | | |
| | (carb^{R}) | | | strand | | |
| pBM23d | Window profiling | SC101 | N.A. | (TC)₁₅ target, bottom | | |
| | (carb^{R}) | | | strand | | |
| pBM23e | Window profiling | SC101 | N.A. | (TC)₁₆ target, botttom | | |
| | (carb^{R}) | | | strand | | |
| pBM23f | Window profiling | SC101 | N.A. | (TC)₁₇ target, bottom | | |
| | (carb^{R}) | | | strand | | |
| pBM23g | Window profiling | SC101 | N.A. | (TC)₁₈ target, bottom | | |
| | (carb^{R}) | | | strand | | |
| pBM13h | NCN assay (spec^{R}) | ColE1 | P_{BAD} | [sd2] UGI-TALE3- | P_{c} | araC |
| | | | | DddA-G1397-N wildtype | | |
| pBM13h- | window profiling (spec^{R}) | ColE1 | P_{BAD} | [sd4U] UGI-TALE3- | | |
| sd4U | | | | DddA-G1397-N wildtype | | |
| pBM13 | NCN assay (spec^{R}) | ColE1 | P_{BAD} | [sd2] UGI-TALE3- | P_{c} | araC |
| | | | | DddA-G1397-N (Q1310R + | | |
| | | | | S1330I + T1380I) | | |
| pBM13- | window profiling (spec^{R}) | ColE1 | P_{BAD} | [sd4U] UGI-TALE3- | P_{c} | araC |
| sd4U | | | | DddA-G1397-N (Q1310R + | | |
| | | | | S1330I + T1380I) | | |
| pBM13a | NCN assay (spec^{R}) | ColE1 | P_{BAD} | [sd2] UGI-TALE3- | P_{c} | araC |
| | | | | DddA-G1397-N (T1314A + | | |
| | | | | G1344R + V1364M + | | |
| | | | | E1370K + T1380I) | | |
| pBM13b | NCN assay (spec^{R}) | ColE1 | P_{BAD} | [sd2] UGI-TALE3- | P_{c} | araC |
| | | | | DddA-G1397-N (N1342S + | | |
| | | | | G1344R + V1364M + | | |
| | | | | E1370K + T1380I) | | |
| pBM13c | NCN assay (spec^{R}) | ColE1 | P_{BAD} | [sd2] UGI-TALE3- | P_{c} | araC |
| | | | | DddA-G1397-N (A1341T + | | |
| | | | | N1342S + E1370K + | | |
| | | | | T1380I) | | |
| pBM13e | NCN assay (spec^{R}) | ColE1 | P_{BAD} | [sd2] UGI-TALE3- | P_{c} | araC |
| | | | | DddA-G1397-N (T1314A + | | |
| | | | | G1344S + E1370K + | | |
| | | | | T1380I) | | |
| pBM13f | NCN assay (spec^{R}) | ColE1 | P_{BAD} | [sd2] UGI-TALE3- | P_{c} | araC |
| | | | | DddA-G1397-N (T1314A + | | |
| | | | | G1344S + E1370K + | | |
| | | | | T1380I + E1396K) | | |
| pBM13g | NCN assay (spec^{R}) | ColE1 | P_{BAD} | [sd2] UGI-TALE3- | P_{c} | araC |
| | | | | DddA-G1397-N (S1330I + | | |
| | | | | A1341V + N1342S + | | |
| | | | | E1370K + T1380I) | | |
| pBM13g- | window profiling (spec^{R}) | ColE1 | P_{BAD} | [sd4U] UGI-TALE3- | P_{c} | araC |
| sd4U | | | | DddA-G1397-N (S1330I + | | |
| | | | | A1341V + N1342S + | | |
| | | | | E1370K + T1380I) | | |
| pBM14d | NCN assay (kan^{R}) | p15A | P_{BAD} | [sd2] UGI-TALE4- | P_{c} | araC |
| | | | | DddA-G1397-C wildtype | | |
| pBM14 | NCN assay (kan^{R}) | p15A | P_{BAD} | [sd2] UGI-TALE4- | P_{c} | araC |
| | | | | DddA-G1397-C (T1413I) | | |
| pBM14c | NCN assay (kan^{R}) | p15A | P_{BAD} | [sd2] UGI-TALE4- | P_{c} | araC |
| | | | | DddA-G1397-C (A1398T + | | |
| | | | | T1413I) | | |
| pBM19 | window profiling (spec^{R}) | p15A | P_{BAD} | [sd4U] UGI-TALE4- | P_{c} | araC |
| | | | | DddA-G1397-C (T1413I) | | |
| SPBM13 | SP (none) | M13 f1 | P_{gIII} | [sd8] UGI-TALE3- | P_{BBa}_J23101 | [sd8] UGI- |
| | | | | DddA-G1397-N wildtype | | TALE3- |
| | | | | | | DddA- |
| | | | | | | G1397-C |
| | | | | | | wildtype |
| SPBM13a | SP (none) | M13 f1 | P_{gIII} | [sd8] UGI-TALE3- | P_{BBa}_J23101 | [sd8] UGI- |
| | | | | DddA-G1397-N (T1380I) | | TALE3- |
| | | | | | | DddA- |
| | | | | | | G1397-C |
| | | | | | | wildtype |
| SPBM14 | SP (none) | M13 f1 | P_{gIII} | [sd8] UGI-TALE3- | P_{BBa}_J23101 | [sd8] UGI- |
| | | | | DddA-G1397-N (E1347A) | | TALE3- |
| | | | | | | DddA- |
| | | | | | | G1397-C |
| | | | | | | wildtype |
| SPBM29 | SP (none) | M13 f1 | P_{gIII} | [sd8] UGI-TALE3- | P_{BBa}_J23101 | [sd8] UGI- |
| | | | | DddA-G1397-N (S1330I + | | TALE3- |
| | | | | A1341V + N1342S + | | DddA- |
| | | | | E1370K + T1380I) | | G1397-C |
| | | | | | | (T1413I) |

### Preparation and Transformation of Chemically Competent Cells

Strain S206034 was used in all phage propagation, plaque assays and PACE experiments. To prepare competent cells, an overnight culture was diluted 100-fold into 50 mL of 2×YT media (United States Biologicals) supplemented with tetracycline and streptomycin and grown at 37° C. with shaking at 230 RPM to OD₆₀₀˜0.4-0.6. Cells were pelleted by centrifugation at 4,000 g for 10 minutes at 4° C. The cell pellet was then resuspended by gentle stirring in 2.5 mL of ice-cold LB media (United States Biologicals) 2.5 mL of 2×TSS (LB media supplemented with 10% v/v DMSO, 20% w/v PEG 3350, and 40 mM MgCl₂) was added. The cell suspension was stirred to mix completely, aliquoted into 100-μL volumes and frozen on dry ice, and stored at −80° C. until use.

To transform cells, 100 μL of competent cells thawed on ice was added to a pre-chilled mixture of plasmid (1-2 μL each; up to 3 plasmids per transformation) in 20 μL 5×KCM solution (500 mM KCl, 150 mM CaCl₂, and 250 mM MgCl₂ in H₂O) and 80 μL H₂O, and stirred gently with a pipette tip. The mixture was incubated on ice for 20 min and heat shocked at 42° C. for 75 s before 600 μL of SOC media (New England BioLabs) was added. Cells were allowed to recover at 37° C. with shaking at 230 RPM for 1.5 h, streaked on 2×YT media+1.5% agar (United States Biologicals) plates containing the appropriate antibiotics, and incubated at 37° C. for 16-18 h.

### Bacteriophage Cloning

For USER assembly of phage, 0.25 pmol of each PCR fragment was added to a make up a final volume of 25 μL. Following USER assembly, the 25 μL USER reaction was transformed into 100 μL chemicompetent S2060 E. coli host cells containing plasmid pJC175e²³ that was modified to include constitutive DddI expression to minimize potential toxicity arising from split DddA expression in bacteria. This plasmid is referred to as pJC175e-DddI. Cells transformed with pJC175e-DddI enables activity-independent phage propagation and were grown overnight at 37° C. with shaking in antibiotic-free 2×YT media. Bacteria were then centrifuged for 2 min at 9,000 g and were plaqued as described below. Individual phage plaques were grown in DRM media (prepared from US Biological CS050H-001/CS050H-003) until the bacteria reached the late growth phase (˜8 hours). Bacteria were centrifuged for 2 min at 9,000 g and the supernatants containing phage were filtered through 0.22 m PVDF Ultrafree centrifugal filter (Millipore) to remove residual bacteria and stored at 4° C.

### Plaque Assays for Phage Titer Quantification and Phage Cloning

Phage were plaqued on S2060³⁴ E. coli host cells containing plasmid pJC175e-DddI (for activity-independent propagation)²³ or host cells transformed with AP and CP for activity dependent propagation (see Table 4 for list of plasmids used in this Example). To prepare a cell stock for plaquing, an overnight culture of host cells (fresh or stored at 4° C. for up to ˜1 week) was diluted 50-fold in 2×YT medium containing appropriate antibiotics and cells were grown at 37° C. to an OD₆₀₀ of 0.8-1.0. Serial dilutions of phage (ten-fold) were made in 2×YT media. To prepare plates, molten 2×YT medium agar (1.5% agar, 55° C.) was mixed with Bluo-gal (4% w/v in DMF) to a final concentration of 0.08% Bluo-gal. The molten agar mixture was pipetted into quadrants of quartered Petri dishes (1.5 mL per quadrant) or wells of a 12-well plate (˜1 mL per well) and was allowed to set. To prepare top agar, a 3:2 mixture of 2×YT medium and molten 2×YT medium agar (1.5%, resulting in a 0.6% agar final concentration) was prepared. To plaque, cell stock (100 or 150 μL for a 12-well plate or Petri dish, respectively) and phage (10 μL) were mixed in 2 mL library tubes (VWR International), and 55° C. top agar was added (400 or 1,000 μL for a 12-well plate or Petri dish, respectively) and mixed one time by pipetting up and down, and then the mixture was immediately pipetted onto the solid agar medium in one well of a 12-well plate or one quadrant of a quartered Petri dish. Top agar was allowed to set undisturbed for 2 min at 25° C., then plates or dishes were incubated, without inverting, at 37° C. overnight. Phage titers were determined by quantifying blue plaques.

### Phage Propagation Assays

S2060 cells transformed with AP and CP plasmids of interest were prepared as described above and were inoculated in DRM. Host cells from an overnight culture in DRM were diluted 50-fold into fresh DRM and were grown at 37° C. to an OD₆₀₀ of 0.3-0.4. Previously titered phage stocks were added to 1 mL of bacterial culture at a final concentration of ˜105 p.f.u. mL⁻¹. The cultures were grown overnight with shaking at 37° C. and were then centrifuged at 4,000 g for 10 min to remove cells. The supernatants were titered by plaquing as described above. Fold enrichment was calculated by dividing the titer of phage propagated on host cells by the titer of phage at the same input concentration shaken overnight in DRM without host cells.

### Phage-Assisted Non-Continuous Evolution Experiments

Host cells transformed with AP and CP were made chemically competent as described above. Chemically competent host cells were transformed with mutagenesis plasmid MP6²⁴ and plated on 2×YT agar containing 10 mM glucose along with appropriate concentrations of antibiotics. Four colonies were picked into 1 mL DRM each in a 96-well deep well plate, and this was diluted 5-fold eight times serially into DRM. The plate was sealed with a porous sealing film and grown at 37° C. with shaking at 230 RPM for 16-18 h. Dilutions with OD600 ˜0.3-0.4 were then treated with 10 mM arabinose to induce mutagenesis. Treated cultures were split into the desired number of 1 mL cultures in a 96-well plate, and inoculated with selection phage at the indicated dilution (see FIG. 23C, FIGS. 25C-25E and FIG. 31D). Infected cultures were grown for 16-18 h at 37° C. and harvested the next day via centrifugation at 4,000×g for 10 min. Supernatant containing evolved phage was isolated and stored at 4° C. Isolated phage were then used to infect the next passage and the process repeated for the duration of the selection. Phage titers were determined by plaque assay.

### Phage-Assisted Continuous Evolution

Unless otherwise noted, PACE apparatus, including host cell strains, lagoons, chemostats, and media, were all used as previously described³⁵. Host cells were prepared as described for PANCE above. Four colonies were picked into 1 mL DRM each in a 96-well deep well plate, and this was diluted 5-fold eight times serially into DRM. The plate was sealed with a porous sealing film and grown at 37° C. with shaking at 230 RPM for 16-18 h. Dilutions with OD₆₀₀ ˜0.4-0.8 were then used to inoculate a chemostat containing 80 mL DRM. The chemostat was grown to OD₆₀₀ ˜0.6-0.8, then continuously diluted with fresh DRM at a rate of 1-1.5 chemostat volumes/h to keep the cell density roughly constant. The chemostat was maintained at a volume of 60-80 mL.

Prior to SP infection, lagoons were continuously diluted with culture from the chemostat at 1 lagoon volume/h and pre-induced with 10 mM arabinose for at least 2 h. Lagoons were infected with SP at a starting titer of 10⁷ pfu/mL and maintained at a volume of 15 mL. Samples (500 μL) of the SP population were taken at indicated times from lagoon waste lines. These were centrifuged at 9,000 g for 2 min, and the supernatant was stored at 4° C. Lagoon titers were determined by plaque assays using S2060 cells transformed with pJC175e-DddI. For Sanger sequencing of lagoons, single plaques were PCR amplified using primers AB1793 (5′-TAATGGAAACTTCCTCATGAAAAAGTCTTTAG (SEQ ID NO: 235)) and AB1396 (5′-ACAGAGAGAATAACATAAAAACAGGGAAGC (SEQ ID NO: 236)) to amplify UGI-TALE3-DddA-G1397-N; Primers AR163 (5′-CCAGCAAGGCCGATAGTTTG (SEQ ID NO: 237)) and AR611 (5′-CTAGCTGATAAATTCATGCCAG (SEQ ID NO: 238)) amplified UGI-TALE3-DddA-G1397-C. Both sets of primers anneal to regions of the phage backbone flanking the evolving gene of interest. Generally, eight plaques were picked and sequenced per lagoon. Mutation analyses were performed using Mutato. Mutato is available as a Docker image at hub.docker.com/r/araguram/mutato

See Sequences, below for sequences of all evolved DddA variants.

### Evolution of Canonical T7-DdCBE for Improved TC Activity

Host cells transformed with AP2, CP2-TCC and MP6 were maintained in an 80 mL chemostat. Four lagoons were each infected with SPBM13a (see Table 4). Upon infection, lagoon dilution rates were increased to 1.5 volume/h. Lagoon dilution rates were increased to 2 vol/h at 20 h and 3 vol/h at 67 h. The experiment ended at 139 h.

### Evolution of T7-DdCBE-CCC-B for Broadened Targeting Scope

Host cells transformed with APi, CP2-CCC and MP6 were maintained in a 50 mL chemostat. Two lagoons were each infected with phage pool CCC-B derived from PANCE. Upon infection, lagoon dilution rates were increased to 1.5 volume/h. Lagoon dilution rates were increased to 2.5 vol/h at 19 h, 3 vol/h at 66 h and 3.5 vol/h at 114h. The experiment ended at 138 h.

### Evolution of T7-DdCBE-GCC-A and T7-DdCBE-GCC-D for Broadened Targeting Scope

Host cells transformed with APi, CP2-GCC and MP6 were maintained in a 50 mL chemostat. One lagoon was infected with phage pool GCC-A and a separate lagoon was infected with phage pool GCC-D. Both phage pools were derived from PANCE. Upon infection, lagoon dilution rates were increased to 1.5 volume/h. Lagoon dilution rates were increased to 2 vol/h at 66 h, 2.5 vol/h at 90 h and 3 vol/h at 114h. The experiment ended at 138 h.

### Bacterial Plasmid Profiling Assay for Context Preference and Editing Window Profiling

To generate NCN target library, 16 μL of plasmids pBM10a to pBM10p were pooled (˜100-200 ng/μL, 1 μL each) and added to 100 μl NEB 10-beta electrocompetent E. coli. To generate TC repeat library for profiling the editing window, 14 μl of plasmids pBM22a-g and pBM23a-g were pooled (˜100-200 ng/μL, 1 μL each) and added to 100 μl NEB 10-beta electrocompetent E. coli. The resulting mixture was incubated on ice for 15 min before transferring into 4×25 μL aliquots in a pre-chilled 16-well Nucleocuvette strip. E. coli cells were electroporated with a Lonza 4D-Nucleofector System using bacterial program X-13. Freshly electroporated E. coli was immediately recovered in 1.4 mL pre-warmed NEB Outgrowth media and incubated with shaking at 200 rpm for 1 h. After recovery, the 1.5 mL culture was divided into two 750 μL aliquots for plating on two 245 mm square dishes (Corning) containing 2×YT medium agar (1.5% agar) mixed with 100 μg/mL of carbenicillin for plasmid maintenance. The dishes were incubated, without inverting, at 37° C. overnight. Colonies were scrapped from the plate the following day and resuspended in 50 mL of 2×YT media. The plasmid library was isolated with a Qiagen Midiprep Kit according to manufacturer's instructions and was eluted in 100 μL H₂O.

To generate the T7-DdCBE-expressing host cells, ˜20-50 μL of NEB 10-beta chemically competent E. coli was transformed with a plasmid from the pBM13 series to express the left-side TALE and a plasmid from the pBM14 series to express the right-side TALE (see Table 4), according to manufacturer's instructions. Cells were plated on 2×YT medium agar (1.5% agar) mixed with 50 μg/mL of spectinomycin, 50 μg/mL of kanamycin and 25 mM glucose. Glucose was added to minimize leaky expression of DdCBE. To make electrocompetent host cells, a single colony of DdCBE-expressing host cells was inoculated in 5-10 mL DRM media and grown at 37° C. with shaking at 200 rpm Cells were grown to OD₆₀₀ ˜0.4, chilled on ice for ˜10 min before centrifuging at 4,000 g for 10 min. Supernatant was discarded and the cell pellet was resuspended with 500-1000 μL of ice-cold 10% glycerol. The process was repeated for four glycerol washes. On the last wash, cells were resuspended in 50 μL of 10% glycerol, mixed with 2 μL of NCN target library (20 ng total) and incubated on ice for 5 min. Cells were transferred into a pre-chilled 16-well Nucleocuvette strip (50 μL per well) and electroporated with a Lonza 4D-Nucleofector System using bacterial program X-5. Freshly electroporated E. coli was immediately recovered in 750 μL of pre-warmed NEB Outgrowth media and recovered by shaking at 200 rpm for 10 min. After recovery, 20-40 mL of DRM was added with 100 μg/mL of carbenicillin, 50 μg/mL of spectinomycin, 50 μg/mL of kanamycin and 10 mM arabinose (to induce DdCBE expression). Cells were incubated with shaking at 200 rpm overnight. Library and base editor plasmids were isolated with a Qiagen Midiprep Kit according to manufacturer's instructions and was eluted in 100 μL H₂O.

### General Mammalian Cell Culture Condition

HEK293T (ATCC CRL-3216), U20S (ATTC HTB-96), K562 (CCL-243) and HeLa (CCL-2) cells were purchased from ATCC and cultured and passaged in Dulbecco's modified Eagle's medium (DMEM) plus GlutaMAX (ThermoFisher Scientific), McCoy's 5A medium (Gibco), RPMI medium 1640 plus GlutaMAX (Gibco), or DMEM plus GlutaMAX (ThermoFisher Scientific), respectively, each supplemented with 10% (v/v) fetal bovine serum (Gibco, qualified). Cells were incubated, maintained, and cultured at 37° C. with 5% CO₂. Cell lines were authenticated by their respective suppliers and tested negative for mycoplasma.

### HEK293T Human Cell Lipofection

Cells were seeded on 48-well collagen-coated plates (Corning) at a density of 1.6- to 2×10⁵ cells/mL 18-24 hours before lipofection in a volume of 250 μl per well. Lipofection was performed at a cell density of approximately 70%. For DdCBE experiments, cells were transfected with 500 ng of each mitoTALE monomer to make up 1000 ng of total plasmid DNA. Lipofectamine 2000 (1.2 μL; ThermoFisher Scientific) was used per well. Cells were harvested 72 h after lipofection for genomic DNA extraction. Unless stated otherwise, the architecture of each DdCBE half is MTS-TALE-[DddA half]-2-amino-acid linker-UGI (see Table 5 for list of TALE binding sites and Sequences, below, for DdCBE sequences).

TABLE 5
List of DNA sequences recognized by TALE proteins

| | | Left | | Right |
|---|---|---|---|---|
| | | SEQ ID | | SEQ ID |
| DdCBE | Left-TALE target sequence | NO: | Right-TALE target sequence | NO: |
| T7 | 5′-T CCGACTTCGCGTT | 351 | 5′-T CGTCGTTTGCT | 364 |
| ND1.1 | 5′-T CTAGCCTAGCCGTTT-3′ | 352 | 5′-T GAGTTTGATGCTCACCCT-3′ | 365 |
| ND1.2 | 5′-T CCTAATGCTT-3′ | 353 | 5′-T ATATAGCCTAGA-3′ | 366 |
| ND2 | 5′-T CTTAGCATACTCCTCAAT-3′ | 354 | 5′-T AGAACTGCTATTATT-3′ | 367 |
| ND4 | 5′-T GCTAGTAACCACGTTCT-3′ | 355 | 5′-T CCTGTAAGTAGGAGAGT-3′ | 368 |
| ND4.2 | 5′-T GAAGCTTCACC-3′ | 356 | 5′-T GGGCGATTATGA-3′ | 369 |
| ND4.3 | 5′-T CTTCAATCAGCC-3′ | 357 | 5′-T GGCTGTTACT-3′ | 370 |
| ND5.2 | 5′-T CAAAACCATACCTCT-3′ | 358 | 5′-T GCTAGGCTGCCAATGT-3′ | 371 |
| (mismatched) | | | | |
| ND5.2 (no | 5′-T CAAAACCATACCTCT-3′ | 358 | 5′-T GCTAGGCTGCCAATGG-3′ | 372 |
| mismatch) | | | | |
| ND5.4 | 5′-T CATAATAGTTACAA-3′ | 359 | 5′-T GCTAGGTGT-3′ | 373 |
| ATP8 | 5′-T ATTAAACACAAACTAT-3′ | 360 | 5′-T ATGGGCTTTGGT-3′ | 374 |
| (mismatched) | | | | |
| ATP8 (no | 5′-T ATTAAACACAAACTAC-3′ | 361 | 5′-T ATGGGCTTTGGT-3′ | 374 |
| mismatch) | | | | |
| ND5.4 | 5′-T CATAATAGTTACAA-3′ | 359 | 5′-T GCTAGGTGT-3′ | 373 |
| SIRT6 | 5′-T TACGCGGCGGGGCTGTC-3′ | 362 | 5′-T CCGGGAGGCCGCACTTG-3′ | 375 |
| JAK2 | 5′-T CTGAAAAAGACTCTGCA-3′ | 363 | 5′-T CCATTTCTGTCATCGTA-3′ | 376 |

### Fluorescence-Activated Cell Sorting (FACS)

Cells treated with the DddA11 variant of ND4.2-DdCBE or ND5.4-DdCBE were seeded on six-well plates (Corning) at a density of 1.8×10⁵ cells/mL 18-24 hours before lipofection in a volume of 2 mL per well. Cells were transfected with 1.25 μg of each mitoTALE monomer to make up 2.5 μg of total plasmid DNA. Lipofectamine 2000 (10 μL; ThermoFisher Scientific) was used per well. Medium was removed 72 h after lipofection, and cells were washed once with 1×Dulbecco's phosphate-buffered saline (ThermoFisher Scientific). Cells were trypsinzed (400 μL; Gibco) and quenched with DMEM media (2 mL). Media was aspirated and cells were resuspended in 1×PBS, filtered through a cell strainer (BD Biosciences), and sorted using a LE-MA900 cell sorter (Sony). See FIGS. 41A-41D for representative FACS sorting examples and Sequences, below, for sequences of P2A linker, eGFP and mCherry.

### U2OS, K562 and HeLa Human Cell Nucleofection

Nucleofection was used for transfection in all experiments using K562, HeLa, and U20S cells. 125 ng of each DdCBE expression plasmid (total 250 ng plasmid) was nucleofected in a final volume of 20 μl in a 16-well nucleocuvette strip (Lonza). K562 cells were nucleofected using the SF Cell Line 4D-Nucleofector X Kit (Lonza) with 5×10⁵ cells per sample (program FF-120), according to the manufacturer's protocol. U20S cells were nucleofected using the SE Cell Line 4D-Nucleofector X Kit (Lonza) with 4×10⁵ cells per sample (program DN-100), according to the manufacturer's protocol. HeLa cells were nucleofected using the SE Cell Line 4D-Nucleofector X Kit (Lonza) with 2×10⁵ cells per sample (program CN-114), according to the manufacturer's protocol. Cells were harvested 72 h after nucleofection for genomic DNA extraction.

Genomic DNA Isolation from Mammalian Cell Culture

Medium was removed, and cells were washed once with 1×Dulbecco's phosphate-buffered saline (ThermoFisher Scientific). Genomic DNA extraction was performed by addition of 40-50 μL freshly prepared lysis buffer (10 mM Tris-HCl (pH 8.0), 0.05% SDS, and proteinase K (20 μg/mL; ThermoFisher Scientific)) directly into the 48-well culture well. The extraction solution was incubated at 37° C. for 60 min and then 80° C. for 20 min. Resulting genomic DNA was subjected to bead cleanup with AMPure DNAdvance beads according to manufacturer's instructions (Beckman Coulter A48705).

### High-Throughput DNA Sequencing of Genomic DNA Samples

Genomic sites of interest were amplified from genomic DNA samples and sequenced on an Illumina MiSeq as previously described¹⁷. Amplification primers containing Illumina forward and reverse adapters (Table 6) were used for a first round of PCR (PCR 1) to amplify the genomic region of interest. Briefly, 1 μL of purified genomic DNA was used as input into the first round of PCR (PCR1). For PCR1, DNA was amplified to the top of the linear range using Phusion Hot Start II High-Fidelity DNA Polymerase (ThermoFisher Scientific), according to the manufacturer's instructions but with the addition of 0.5×SYBR Green Nucleic Acid Gel Stain (Lonza) in each 25 μL reaction. For all amplicons, the PCR1 protocol used was an initial heating step of 2 min at 98° C. followed by an optimized number of amplification cycles (10 s at 98° C., 20 s at 62° C., 30 s at 72° C.). Quantitative PCR was performed to determine the optimal cycle number for each amplicon. The number of cycles needed to reach the top of the linear range of amplification are ˜17-19 cycles for mtDNA amplicons and ˜27-28 cycles for nuclear DNA amplicons. Barcoding PCR2 reactions (25 μL) were performed with 1 μL of unpurified PCR1 product and amplified with Phusion Hot Start II High-Fidelity DNA Polymerase (ThermoFisher Scientific) using the following protocol 98° C. for 2 min, then 10 cycles of [98° C. for 10 s, 61° C. for 20 s, and 72° C. for 30 s], followed by a final 72° C. extension for 2 min. PCR products were evaluated analytically by electrophoresis in a 1.5% agarose gel. After PCR2, up to 300 samples with different barcode combinations were combined and purified by gel extraction using the QIAquick Gel Extraction Kit (QIAGEN). DNA concentration was quantified using the Qubit ssDNA HS Assay Kit (Thermo Fisher Scientific) to make up a 4 nM library. The library concentration was further verified by qPCR (KAPA Library Quantification Kit-Illumina, KAPA Biosystems) and sequenced using an Illumina MiSeq with 210- to 300-bp single-end reads. Sequencing results were computed with a minimum sequencing depth of approximately 10,000 reads per sample.

TABLE 6
Primers used for mammalian and bacteria cell genomic DNA amplification at sites targeted
by DdCBEs

| | | Forward | | Reverse |
|---|---|---|---|---|
| | | SEQ ID | | SEQ ID |
| Site | HTS forward primer | NO: | HTS reverse primer | NO: |
| ND1.1 | ACACTCTTTCCCTACACGACGCTCT | 239 | TGGAGTTCAGACGTGTGCTCTT | 269 |
| | TCCGATCTNNNNCTCACCATCGCT | | CCGATCTGGCTAGGGTGACTTC | |
| | CTTCTACTATG | | ATATGAG | |
| ND1.2 | ACACTCTTTCCCTACACGACGCTCT | 240 | TGGAGTTCAGACGTGTGCTCTT | 270 |
| | TCCGATCTNNNNGCCAACCTCCTA | | CCGATCTGGGCGGTGATGTAGA | |
| | CTCCTCAT | | GGG | |
| ND2 | ACACTCTTTCCCTACACGACGCTCT | 241 | TGGAGTTCAGACGTGTGCTCTT | 271 |
| | TCCGATCTNNNNCGTAAGCCTTCT | | CCGATCTGTTGAGTAGTAGGAA | |
| | CCTCACT | | TGCGGTAG | |
| ND4 | ACACTCTTTCCCTACACGACGCTCT | 242 | TGGAGTTCAGACGTGTGCTCTT | 272 |
| | TCCGATCTNNNNGACTTCAAACTC | | CCGATCTGTTGTGGTAAATATG | |
| | TACTCCCACTAATAG | | TAGAGGGAG | |
| ND4.2 | ACACTCTTTCCCTACACGACGCTCT | 243 | TGGAGTTCAGACGTGTGCTCTT | 273 |
| ND4.3 | TCCGATCTNNNNCTGCCTACGACA | | CCGATCTTGGGAGTAGAGTTTG | |
| | AACAGACC | | AAGTCC | |
| ND5.2 | ACACTCTTTCCCTACACGACGCTCT | 244 | TGGAGTTCAGACGTGTGCTCTT | 274 |
| | TCCGATCTNNNNCGGGTCCATCAT | | CCGATCTAGAGTAATAGATAGG | |
| | CCACAAC | | GCTCAGGC | |
| ND5.4 | ACACTCTTTCCCTACACGACGCTCT | 245 | TGGAGTTCAGACGTGTGCTCTT | 275 |
| | TCCGATCTNNNNGCAGTCTGCGCC | | CCGATCTCGAATATCTTGTTCAT | |
| | CTTAC | | TGTTAAGGTTG | |
| ATP8 | ACACTCTTTCCCTACACGACGCTCT | 246 | TGGAGTTCAGACGTGTGCTCTT | 276 |
| | TCCGATCTNNNNCTTTACAGTGAA | | CCGATCTGGGGGCAATGAATGA | |
| | ATGCCCCAAC | | AGCG | |
| SIRT6 (on- | ACACTCTTTCCCTACACGACGCTCT | 247 | TGGAGTTCAGACGTGTGCTCTT | 277 |
| target) | TCCGATCTNNNNGGAAGCGGCCTC | | CCGATCTGCCCCCTGATATTCC | |
| | AACAAG | | CAC | |
| JAK2 (on- | ACACTCTTTCCCTACACGACGCTCT | 248 | TGGAGTTCAGACGTGTGCTCTT | 278 |
| target) | TCCGATCTNNNNGCTAGGATTACA | | CCGATCTCACCTGAAGAACTGG | |
| | GGTGTGAGAC | | ATCTATTTG | |
| T7-DdCBE (for | ACACTCTTTCCCTACACGACGCTCT | 249 | TGGAGTTCAGACGTGTGCTCTT | 279 |
| targeting NCN | TCCGATCTNNNNCGTTTTAGACTG | | CCGATCTCGGATGCCGGGAGC | |
| library plasmid | AGCACGTCAATAC | | | |
| SIRT6-CT1 | ACACTCTTTCCCTACACGACGCTCT | 250 | TGGAGTTCAGACGTGTGCTCTT | 280 |
| | TCCGATCTNNNNTGGCCAAGCAGG | | CCGATCTCTGTTGCCTCCTTGGG | |
| | GATC | | TAC | |
| SIRT6-CT2 | ACACTCTTTCCCTACACGACGCTCT | 251 | TGGAGTTCAGACGTGTGCTCTT | 281 |
| | TCCGATCTNNNNGGGGGAGTCGGA | | CCGATCTCTTGGCACACAGCAG | |
| | CTTAGAAGG | | GTGCTC | |
| SIRT6-CT3 | ACACTCTTTCCCTACACGACGCTCT | 252 | TGGAGTTCAGACGTGTGCTCTT | 282 |
| | TCCGATCTNNNNTGCTAGGCCCCG | | CCGATCTCCCCACTTCCTTCCTC | |
| | ATTTGTGG | | ACCCT | |
| SIRT6-CT4 | ACACTCTTTCCCTACACGACGCTCT | 253 | TGGAGTTCAGACGTGTGCTCTT | 283 |
| | TCCGATCTNNNNGACCCTTCCATT | | CCGATCTGCTCTGGCTGGAGCA | |
| | AGATCTGAGGGC | | CAGGAA | |
| SIRT6-CT5 | ACACTCTTTCCCTACACGACGCTCT | 254 | TGGAGTTCAGACGTGTGCTCTT | 284 |
| | TCCGATCTNNNNGGATCCAAGGTG | | CCGATCTACCACAGAGCCTCAC | |
| | CCTTCACCC | | CCACCA | |
| SIRT6-CT6 | ACACTCTTTCCCTACACGACGCTCT | 255 | TGGAGTTCAGACGTGTGCTCTT | 285 |
| | TCCGATCTNNNNGCTGGATTCGAT | | CCGATCTCAGGCCCTACTGGGA | |
| | CTGAGGTCAGCT | | CGAACA | |
| SIRT6-CT7 | ACACTCTTTCCCTACACGACGCTCT | 256 | TGGAGTTCAGACGTGTGCTCTT | 286 |
| | TCCGATCTNNNNCCCAGGACAAAG | | CCGATCTCTCTTGCACAAGGTT | |
| | TTGCTTTGGGGC | | GCCACCTC | |
| SIRT6-CT8 | ACACTCTTTCCCTACACGACGCTCT | 257 | TGGAGTTCAGACGTGTGCTCTT | 287 |
| | TCCGATCTNNNNGCCTCGCCAAGA | | CCGATCTATTCCGGGTAGGCGA | |
| | AACGCCCAT | | GGAGGT | |
| SIRT6-CT9 | ACACTCTTTCCCTACACGACGCTCT | 258 | TGGAGTTCAGACGTGTGCTCTT | 288 |
| | TCCGATCTNNNNCCAGCACACCCT | | CCGATCTAGGGCCCCAGTGAGT | |
| | GATAAGC | | G | |
| SIRT6-CT10 | ACACTCTTTCCCTACACGACGCTCT | 259 | TGGAGTTCAGACGTGTGCTCTT | 289 |
| | TCCGATCTNNNNCCAGGGCAACAG | | CCGATCTGCCCACATCAGCTCT | |
| | GTTTAAG | | GC | |
| JAK2-CT1 | ACACTCTTTCCCTACACGACGCTCT | 260 | TGGAGTTCAGACGTGTGCTCTT | 290 |
| | TCCGATCTNNNNGTGTGGTCAGGA | | CCGATCTCTCCAGCCTGGGCGA | |
| | CAAACTGTTCCC | | CTGAAT | |
| JAK2-CT2 | ACACTCTTTCCCTACACGACGCTCT | 261 | TGGAGTTCAGACGTGTGCTCTT | 291 |
| | TCCGATCTNNNNGGGGCTGGGAGA | | CCGATCTCCACTGATAACCTAT | |
| | AGAGAGGAA | | GCCCAGGA | |
| JAK2-CT3 | ACACTCTTTCCCTACACGACGCTCT | 262 | TGGAGTTCAGACGTGTGCTCTT | 292 |
| | TCCGATCTNNNNGTCCACTCTGTG | | CCGATCTCTATGGAGACTCCTT | |
| | TTACCCAGATC | | CAGGTCTTTTTTCCC | |
| JAK2-CT4 | ACACTCTTTCCCTACACGACGCTCT | 263 | TGGAGTTCAGACGTGTGCTCTT | 293 |
| | TCCGATCTNNNNGCCGGTTTAGGT | | CCGATCTGAGTGAGACCCTGTC | |
| | ATGGGAACCAC | | TTGGGGA | |
| JAK2-CT5 | ACACTCTTTCCCTACACGACGCTCT | 264 | TGGAGTTCAGACGTGTGCTCTT | 294 |
| | TCCGATCTNNNNGGAACAGTGATC | | CCGATCTGGCTCTGAAGAAAGT | |
| | AGTTCTGTTACCTGAC | | ACGTAAAAGACCAAG | |
| JAK2-CT6 | ACACTCTTTCCCTACACGACGCTCT | 265 | TGGAGTTCAGACGTGTGCTCTT | 295 |
| | TCCGATCTNNNNTCCGGATTCTCC | | CCGATCTCCCAGGACAGCTTTG | |
| | TGCTGAGGC | | AATGTGGC | |
| JAK2-CT7 | ACACTCTTTCCCTACACGACGCTCT | 266 | TGGAGTTCAGACGTGTGCTCTT | 296 |
| | TCCGATCTNNNNCCGACTGCTCTG | | CCGATCTGCCCATGTATTGGGG | |
| | CCTTCTGA | | CATAACC | |
| JAK2-CT8 | ACACTCTTTCCCTACACGACGCTCT | 267 | TGGAGTTCAGACGTGTGCTCTT | 297 |
| | TCCGATCTNNNNGTCCATGAATGA | | CCGATCTGCTCTGGCTCTTGCA | |
| | CAGAGCAC | | GAC | |
| JAK2-CT9 | ACACTCTTTCCCTACACGACGCTCT | 268 | TGGAGTTCAGACGTGTGCTCTT | 298 |
| | TCCGATCTNNNNGTAAGGGTGAAA | | CCGATCTCAAGTTCCTGGAATG | |
| | ATTCATTTGATGG | | CTGCATG | |

### High-Throughput Sequencing of NCN and TC Repeat Library Plasmids

Primers T7-DdCBE Fwd and T7-DdCBE Rev were used to amplify the region containing the NCN target spacing region (see Table 6). Briefly, 100 ng of purified plasmids were used as input into the first round of PCR (PCR1) in a total reaction volume of 50 μL. For PCR1, quantitative PCR was used to amplify DNA to the top of the linear range as described above (˜14 cycles). PCR1 products were purified with QIAquick PCR Purification kit according to manufacturer's instructions and eluted in 20 μL. Barcoding PCR2 reactions (50 μL) were performed with 10 μL of purified PCR1 product and amplified for 8 cycles. Subsequent steps after PCR2 are as described above.

### Analysis of HTS Data for DNA Sequencing and Targeted Amplicon Sequencing

Sequencing reads were demultiplexed using MiSeq Reporter (Illumina). Batch analysis with CRISPResso2³⁶ was used for targeted amplicon and DNA sequencing analysis (see Table 7 for list of amplicon sequences used for alignment. A 10-bp window was used to quantify indels centered around the middle of the dsDNA spacing. To set the cleavage offset, a hypothetical 15- or 16-bp spacing region has a cleavage offset of −8. Otherwise, the default parameters were used for analysis. The output file “Reference.NUCLEOTIDE_PERCENTAGE_SUMMARY.txt” was imported into Microsoft Excel for quantification of editing frequencies. Reads containing indels within the 10-bp window are excluded for calculation of editing frequencies. The output file “CRISPRessoBatch_quantification_of_editing_frequency.txt” was imported into Microsoft Excel for quantification of indel frequencies. Indel frequencies were computed by dividing the sum of Insertions and Deletions over the total number of aligned reads.

TABLE 7
Amplicons for high-throughput sequencing analyses

| | | SEQ ID |
|---|---|---|
| Site | Amplicon | NO: |
| ND1 | CTCACCATCGCTCTTCTACTATGAACCCCCCTCCCCATACCCAACCCCCTGGT | 299 |
| | CAACCTCAACCTAGGCCTCCTATTTATTCTAGCCACCTCTAGCCTAGCCGTTT | |
| | ACTCAATCCTCTGATCAGGGTGAGCATCAAACTCAAACTACGCCCTGATCGG | |
| | CGCACTGCGAGCAGTAGCCCAAACAATCTCATATGAAGTCACCCTAGCC | |
| ND1.2 | GCCAACCTCCTACTCCTCATTGTACCCATTCTAATCGCAATGGCATTCCTAAT | 300 |
| | GCTTACCGAACGAAAAATTCTAGGCTATATACAACTACGCAAAGGCCCCAAC | |
| | GTTGTAGGCCCCTACGGGCTACTACAACCCTTCGCTGACGCCATAAAACTCTT | |
| | CACCAAAGAGCCCCTAAAACCCGCCACATCTACCATCACCCTCTACATCACC | |
| | GCCC | |
| ND2 | CGTAAGCCTTCTCCTCACTCTCTCAATCTTATCCATCATAGCAGGCAGTTGAG | 301 |
| | GTGGATTAAACCAAACCCAGCTACGCAAAATCTTAGCATACTCCTCAATTAC | |
| | CCACATAGGATGAATAATAGCAGTTCTACCGTACAACCCTAACATAACCATT | |
| | CTTAATTTAACTATTTATATTATCCTAACTACTACCGCATTCCTACTACTCAAC | |
| ND4 | GACTTCAAACTCTACTCCCACTAATAGCTTTTTGATGACTTCTAGCAAGCCTC | 302 |
| | GCTAACCTCGCCTTACCCCCCACTATTAACCTACTGGGAGAACTCTCTGTGCT | |
| | AGTAACCACGTTCTCCTGATCAAATATCACTCTCCTACTTACAGGACTCAACA | |
| | TACTAGTCACAGCCCTATACTCCCTCTACATATTTACCACAAC | |
| ND4.2 | CTGCCTACGACAAACAGACCTAAAATCGCTCATTGCATACTCTTCAATCAGCC | 303 |
| ND4.3 | ACATAGCCCTCGTAGTAACAGCCATTCTCATCCAAACCCCCTGAAGCTTCACC | |
| | GGCGCAGTCATTCTCATAATCGCCCACGGACTTACATCCTCATTACTATTCTG | |
| | CCTAGCAAACTCAAACTACGAACGCACTCACAGTCGCATCATAATCCTCTCTC | |
| | AAGGACTTCAAACTCTACTCCCA | |
| ND5.2 | CGGGTCCATCATCCACAACCTTAACAATGAACAAGATATTCGAAAAATAGGA | 304 |
| | GGACTACTCAAAACCATACCTCTCACTTCAACCTCCCTCACCATTGGCAGCCT | |
| | AGCATTAGCAGGAATACCTTTCCTCACAGGTTTCTACTCCAAAGACCACATCA | |
| | TCGAAACCGCAAACATATCATACACAAACGCCTGAGCCCTATCTATTACTCT | |
| ND5.4 | GCAGTCTGCGCCCTTACACAAAATGACATCAAAAAAATCGTAGCCTTCTCCA | 305 |
| | CTTCAAGTCAACTAGGACTCATAATAGTTACAATCGGCATCAACCAACCACA | |
| | CCTAGCATTCCTGCACATCTGTACCCACGCCTTCTTCAAAGCCATACTATTTA | |
| | TGTGCTCCGGGTCCATCATCCACAACCTTAACAATGAACAAGATATTCG | |
| ATP8 | CTTTACAGTGAAATGCCCCAACTAAATACTACCGTATGGCCCACCATAATTAC | 306 |
| | CCCCATACTCCTTACACTATTCCTCATCACCCAACTAAAAATATTAAACACAA | |
| | ACTACCACCTACCTCCCTCACCAAAGCCCATAAAAATAAAAAATTATAACAA | |
| | ACCCTGAGAACCAAAATGAACGAAAATCTGTTCGCTTCATTCATTGCCCCC | |
| SIRT6 | GGAAGCGGCCTCAACAAGGGAAACTTTATTGTTCCCGTGGGGCAGTCGAGGA | 307 |
| (on-target) | TGTCGGTGAATTACGCGGCGGGGCTGTCGCCGTACGCGGACAAGGGCAAGTG | |
| | CGGCCTCCCGGAGGTGAGCGCGTCTGAGGGTCCCGAGCATCGCGCCCCAGGG | |
| | CCGCGGGCTGGGAGCGGCCGGATGGCAGGGGGGCATTGTGGGAATATCAGG | |
| | GGGC | |
| JAK2 (on- | GCTAGGATTACAGGTGTGAGACACTGCGCCCAGCCCATTTGTAACTTTATTGT | 308 |
| target) | TTTCTCTTACAGGCAAATGTTCTGAAAAAGACTCTGCATGGGAATGGCCTGCC | |
| | TTACGATGACAGAAATGGAGGGAACATCCACCTCTTCTATATATCAGAATGG | |
| | TGATATTTCTGGAAATGCCAATTCTATGAAGCAAATAGATCCAGTTCTTCAGG | |
| | TG | |
| pBM10 | CGTTTTAGACTGAGCACGTCAATACGCAAACCGCCTCTCCCCGCGCGTTGGCC | 309 |
| plasmid | GATTCATTAATGCAGCTGGCACGACAGGTTTCCCGACTGGAGTTTGTAGTCTA | |
| series for | GTCTGTCCGACTTCGCGTTTTTTTTGTTTATTTCAGCAAACGACGAACCCGC | |
| NCN | CAAACGCGCCCTGACGGGATTGTCTGCTCCCGGCATCCG | |
| library | Blue = UMI; Bold = TALE binding sites; Underlined = NCN target | |
| SIRT6- | TGGCCAAGCAGGGATCACGTCCCTCTGCCTTGGCCACTCCAGGCCACCTGCA | 310 |
| CT1 | GAGGGGGCAGCCCTGTCAGTGATGGAGGGGGAGACAGCCTGTGTTATTCACG | |
| | CCGGAAGTCCATGGCATTCTCAGAGAGCTCTTCCCCGTACCTCCCCAGAACAC | |
| | TGAGAATGGATGAATCAGTCCCGGCTCATCAGAGCCTTTCATGCTAAACCAG | |
| | CCCAAATGACTGATTTAGAAACTGCGTGGTCTTGTTCTTTGAGTGGATTTCTG | |
| | AGGCACACTCCAGAGCAGGCCCAGGTCTTCATTATCTCCATGCACACCCTTTG | |
| | ATTAAGCATCTGTGACCACACAGCACTGTGTGCTGTACCCAAGGAGGCAACA | |
| | G | |
| SIRT6- | GGGGGAGTCGGACTTAGAAGGTTGCCTCTGCTGCTCTCCCACTAAGAACAAT | 311 |
| CT2 | GCAGCTGCCCAGGAAACACACCCGAGCTATGAATAGGCTCAAGCACACAGG | |
| | GCGAAGCTGTCGCATTTCTGCCGTTTAGAAAGGATCTCTAGTGTGAAGTTGCT | |
| | TGGAGGCTCTGATTTTGCAAACCCAATTAAATATAGATTTCTTTGGGGTGACG | |
| | GTTGGGAAGGGTGTGGGTGGAATGTATGTCTGTGCTGCAATGCAATTTAATC | |
| | CTGAAAGTTTCAGCAAACCTATTCACACACTCCATAAACATTTCTGAGCACCT | |
| | GCTGTGTGCCAAG | |
| SIRT6- | TGCTAGGCCCCGATTTGTGGACACTGAGCTAGGCAAAAGGATATATAAAGAG | 312 |
| CT3 | ATGCAAGCTAGAGGAGGGGAAAGTTCTGGATGGGGACCTCATTGGCCTCCTC | |
| | ACCTCTGTAGACCCACGTCTTGGGTTGACCCAGGGTAGAACCCTATAAGGAT | |
| | TTGTTCAATAAACGAATGCATAGAGACTGATCAACAACTGACAAAGTGGTAT | |
| | TTTACAGATGCCGTGAAAGCAGCATACAAGCCGGAGAGCCTACTGGAAGCAA | |
| | GAGGGGAAAACCCAGCCGGGTATGGGGGCTGCGGGGAGGATGCCAGGTGGC | |
| | GGAGACTGGATTCAGGATTTGATGCTTGAACTGAACCTTCTTTTAAGAGGTGA | |
| | GTGGGCTGGGAGGTTGTGATGCCCAGAGGGTGAGGAAGGAAGTGGGG | |
| SIRT6- | GACCCTTCCATTAGATCTGAGGGCGGGGTGGGAGGGCAGAGGAGGGGAGGA | 313 |
| CT4 | CAGAGAAGTGGGAGGAAGCAGGAGAGGGGGTGGAGGGGTAGGGGCACAGT | |
| | GTCATGCCTCTCGGGCTTCCCCCACAGGGTGAATGACTGTGTGCTGCGGGTGA | |
| | ATGAGGTGGACGTGTCGGAGGTGGTACACAGCCGGGCGGTGGAGGCGCTGA | |
| | AGGAGGCAGGCCCTGTGGTGCGATTGGTGGTGCGGAGGCGACAGCCTCCACC | |
| | CGAGACCATCATGGAGGTCAACCTGCTCAAAGGGCCCAAAGGTGCGGCCCTC | |
| | CAGGTTCCTGTGCTCCAGCCAGAGC | |
| SIRT6- | GGATCCAAGGTGCCTTCACCCTGCGGCCTGCTCTATGAGGGCCTGCGTCGCAC | 314 |
| CT5 | ATCAGCTGTTGGTATCCTCACCTCGCTGAGCCGTTCCTCCGAGTGTGCTCTGT | |
| | GGCTTGCACCAGGCCTAGCTGGGCCTTGTGTCTGGTCTCATCCAGCGTCTTCT | |
| | GATTGTGGGGAATGAGGCAGAGGTCTCCCCACCTGGGCTGTCCGTGTGGCGA | |
| | AAGCCTTCCGGGGGGAGGGAGAGGAAGTGCCTCTACAAGGGCCTAGAGTCG | |
| | TCCTGTGGCACTGAGGGCTCTGGCAGCTGCACACAGGTTGATAATGTCACTG | |
| | GTGGGTGAGCTCTGTGGT | |
| SIRT6- | GCTGGATTCGATCTGAGGTCAGCTTGACTTTTCCTTCAGGATGATGCGCTGCT | 315 |
| CT6 | TCCTCTGATGAAGGATCAGGGATGCAGAATGGGACTGATGTATCTGTCCAAT | |
| | CATCCCACTCATTCTCCTCACTTTGCATGCTCTTCTCGGGGCCATCTACAGAC | |
| | AGGTGAGGGGCCCCAGGCAGACAGCTCCTTGGGACACTGAGCTCCACATTGA | |
| | AATGTTACTCAAATGAATGCGCAGAGGAGCTGTGAGTGAGAAAGTAAGCCCT | |
| | CCAGCGTGAAGAGCCCCGGAAAAGCCACCCTTGCTCTGGCACCTGTCATGCA | |
| | GCCCTCACAGCAGTACTCCCATGGGGTATGGGAGGACAGGGGCACTTCTGTT | |
| | GGTCTTTCCTTCCTCATGTTCGTCCCAGTAGGGCCTG | |
| SIRT6- | CCCAGGACAAAGTTGCTTTGGGGCGCTTACAGGTTGAATCACCCATAGCTAT | 316 |
| CT7 | GTTTTGTTTGGCTTGTACTGTGTTGTTTACATTATTTTATTAGTTGGCAATAGC | |
| | TTTAAATCAAAATTTACATTAAGGTCCAGATTTGGGGCTCCTCTTAAAAAAAT | |
| | AAATCAGCACATCTACAACAGTAGGATTTTCACGCCCAATGGCAAAATCTGT | |
| | TAAAGCTGTGTGACAGCTTCTCCTTTTAATGGAGATGACCTCTTCAGACCCTG | |
| | TACTCCAGGTGCCACAGGAACCAGCTGCCCGGTTCATCCTTGTGTGTTACCTG | |
| | CATGAGGTGGCAACCTTGTGCAAGAG | |
| SIRT6- | GCCTCGCCAAGAAACGCCCATTTATTTCCTTCCTATTTATTTATTTTGCAAAAG | 317 |
| CT8 | CCGCCTTTTGAAAGCCGGAGTGCAGTGCGGACTGGCTGAGGCCGGGGCCCAG | |
| | GCGCGCGCCTCCTTCCTGCAGGCGGCCCGGGGCATCGATCGCGCGGGGCGTA | |
| | ATGAACCCTAATAACAGCTCTATCACCGCCCGCCCGCCAATTGGCCCGGGTG | |
| | CCCTCCAAATTAGCCACAAAGAAGCCAGTCTGTCAATATTAATTCCGCCGCG | |
| | GAGATTACTTGTTGTGGGAGAACAAAGGTGTTCTGGCTTGAGCTGAACAACT | |
| | TAACTCCTTGTCTCATAAAATTTAAGCTGCTATTGATCGCCTCCTTGCATTGTG | |
| | TGAGGATTAAACACGTGCGCGTGCACATCCAGGCACACGCGCGCACACACTC | |
| | GGGGCGCCCGGCCCCGCTCAGGCAGCCGCCCCACCTCCTCGCCTACCCGGAA | |
| | T | |
| SIRT6- | CCAGCACACCCTGATAAGCAGGATTCAGATTGGGCATGGGACAGGACAAAG | 318 |
| CT9 | GCTCTGAGGAGGCATGAGTGGAGTAGTAGAGAGAACACAGGACTCTGGGTTC | |
| | TGAGCCAAGCTCTGCCATCCTGCAGCCATCTGACCCCTGGCCAGGCCTTCCCC | |
| | CAACTTCCTCAGCTGTGAAACGCGGCAGGGCTGCAGATGACGTAGGTGGGCA | |
| | GAGGCCCTCTGGTGCCTACTGCTAGACACCCTTCTACTCTGGTGGGAGACCTT | |
| | GTGCTCAAACGCTCTCAGAAGTCAGGGCAGTTGGGAGTCCAGGTCACACACA | |
| | CTCACTGGGGCCCT | |
| SIRT6- | CCAGGGCAACAGGTTTAAGACCCATTAGGAGACAATATTGTCGTCATGAATA | 319 |
| CT10 | TAAATGAGGCCTAGGCAGGTTGGGAGTAGCTAGTGTCTTGGCTCTGGCACAA | |
| | CCAGGCCCTGTTCCTCCACAGGGCTGGCCAGGGCAGAGGTGAAAGGGGCTGG | |
| | GGTGTGACCGAGTGAGCTGGGGTAGAGGTAGAAAAAGGGACACAGAACATC | |
| | CCAGGCCTAAGCTACATGGAGTATGAGAGAACAGCTGGAATGTGCTCTAGAA | |
| | GCCCTGGGAGAAGGCCTCAGTTGAGGCTGAATTCAGATATGCCTGGGACCCG | |
| | GTCTCTGTTAAGAGACCCCGGAGAGCTGGTGCAGAGCTGATGTGGGC | |
| JAK2- | GTGTGGTCAGGACAAACTGTTCCCTGAACTTAAAAGGTGAAGGACAAGACCC | 320 |
| CT1 | CATATTATTATCCTGTATTAAAAAAGGAAATATACATATATGTACACAGACA | |
| | CCCCATATCACAGACAAGAAACTTCCCATAATTCAAAGGGAGACCATTTCCT | |
| | TATTAGCAAAGGTGCGCATTACAGTATTTCATGACAGTTAAAAATTACACAC | |
| | CTACCACCTGTTTTGGTCAATCTTGCTAAAAAAGACACTGAAATAGACAATTT | |
| | TCTTCTTTAAGGTAAAGACAATGTCTAATTTAGAAAATCTTCCTCTTGAGATT | |
| | GCACAGTGAAAGGTATCAGTTAATTAAATATAAAAGCTTACCGTTTTTGTTTT | |
| | TTTTTGAGACAGAGTTTTATTCAGTCGCCCAGGCTGGAG | |
| JAK2- | GGGGCTGGGAGAAGAGAGGAATGGGAGTTATTGATAATGGGTACAGTTTCA | 321 |
| CT2 | GTTTGAAAAGATGAAAAATTCTGAAGATGGATGGTGGTAGTGATTATACAAC | |
| | AATGTGAATATACTTAATGCTGCTGAACTATACAGTTTAAAATGGTTAAATGG | |
| | TACATTTTATGTATACGTTACCATAAAAAAAAAAGTCTGGACTATAATGTTAG | |
| | AAGTAAAGATAGTAGTAATCTTATAGGAGAAAGAAGAGGATGGTGTTCAGA | |
| | AAGAGCGTGGTGAGGGTGGGGAACTTCAAGGGTGCTGGCAATATTCTATTCC | |
| | TGGGCATAGGTTATCAGTGG | |
| JAK2- | GTCCACTCTGTGTTACCCAGATCTTGTACAGGAAATGGAGTGTATTTAATATT | 322 |
| CT3 | GATGACTCTGTATGTGTCATTTAATTGTCTCTATTGATTGGATTTCATCAGAGT | |
| | GTGGGGCTTGGACAGAGCTTTGATTAGACTGTAAGGATTCTTTGCCGTTTTCT | |
| | TTTTTCCGACACCATATCAATGTACCTTCTGGTGTGATATCACTTTCACAATCA | |
| | ATATCTGAAAAAAGCTCTGCACACAGGCCTGCAGGAAAAGAGAGAAGGCAC | |
| | TGCCTCTAAGTCCCCTTATACACATAGATTTAACCTCTCCAATTGAAAAAGGT | |
| | TTATTGCATGTTTTAGAGCAGGATGGAGTGGGAAAAAAGACCTGAAGGAGTC | |
| | TCCATAG | |
| JAK2- | GCCGGTTTAGGTATGGGAACCACACAACCTCTAAAGTGAAGGAGGCTTCTCC | 323 |
| CT4 | AGCAAAACCGAACGGCATATTCAGATGCTGGGTGAGGATTCATAATATTTAG | |
| | CTATTTTGTTAATTAATATAACAGTAATTTCTTTATTCAGTCACAGAAACATC | |
| | AATGTATCTATGCTGAAGCACCATAATTTTATAAGCACAGTTATTGGGGGAA | |
| | AATGTTACCTTTTTGTCCTAAAAAGACTCTCCACGCATGTAGCTAGGGAAAGC | |
| | TAAGGTCAGTGGCAGAGTTATATGCACACCTCCACCACCAACATCACCACCA | |
| | CCACCTCCTCCTTCATCTCTCATCCAGCTCTGCCTTAGGCCCTTTTTTTTCTTTT | |
| | CTTTTCTTTTCTTTTTTTTTTCCCCAAGACAGGGTCTCACTC | |
| JAK2- | GGAACAGTGATCAGTTCTGTTACCTGACCATAATGGCTAAACAAAACAACCA | 324 |
| CT5 | AATTATAAATTAAAAGGGGTTTGAATATATAGAACATTCATTTTTCAGCTACT | |
| | AGCCAAAAAATACTATCTCTACCTGCTATCCCATGTGTTGTGTTCTTTTTAGA | |
| | CTGTCTAATTTTGAGCTGAACTGTTTCTTAGACTGACAGACAAGTCATAAATC | |
| | AAGTGATTGCTATAAATGCTTTTTATTTATATAGAGAGTGTCTCAGATTTTTA | |
| | CCTTTCTTTCATAAACTTGTCATAAATTTCTAATTCACCAATAGATGGTTGGTG | |
| | TCCATTTTTCTTGAGTCTAAGACACTTTTAAAAATTTACTTGACTTGGTCTTTT | |
| | ACGTACTTTCTTCAGAGCC | |
| JAK2- | TCCGGATTCTCCTGCTGAGGCAGGAGAATCGCTTGAAGCTGGGAGGCAGAGA | 325 |
| CT6 | TTGCAGTGAGCTAAGATCAGGCCATTGCACTCCAGCCTGGGTGACAGAGCGA | |
| | GACTCTGTCTCAAAAAAACAAAACAAAACAAAAAAAAAGAGAAAGTCAGTA | |
| | GCATGTAGATCAGGAGTGTCCAATCTTTTGGCTCCTCTGGAAGAAGAATTGTC | |
| | TTGGGCCACACATAAAATACACCATCACCTAACGATAGCTGATGAGCTTAAA | |
| | AAAAATCACAGAAAAATATCATAATGTTTTAAGAATGTTTATGAATTTTTTAC | |
| | AAATTTCTGTTGGGCCACATTCAAAGCTGTCCTGGG | |
| JAK2- | CCGACTGCTCTGCCTTCTGAATCATATGTAACCAAATCAAGTCAAACAGGTTA | 326 |
| CT7 | GAAGACAACTCACACTTCAGTGTCATCTGTACTCTTATCTTCATGAGTGTGGG | |
| | AATGTACAATCCACTTTCGCTCACTATATTAATTCATTCTGGTTCCTCATGTAC | |
| | TTAACCTATTTTTATTTTTTCAGTTTGGATACAACCCAAATCCTCTCAAGCCTT | |
| | TTAAATGCAAAAAAAAAAAATAAATTTAAAGTATATGTAGTTAAAAATACTC | |
| | ATGTCTTTACCCATTCCTTTGAGAATTTCTGTAGAGGCTTTCTCAAAATGCAG | |
| | AGAGTGGAGGCAGTCATAACATATGATGCCTGCAGATTGGGGTATCTGTCAT | |
| | TTAATCAAGAGAAGAAATAAACATTTTATGTCATTGATTTATCCATTTTAGTT | |
| | GCTATCATCTTTATAGGTTATGCCCCAATACATGGGC | |
| JAK2- | GTCCATGAATGACAGAGCACATTCACACTCATTCATTTACATATTTTCTATGC | 327 |
| CT8 | CTGTTTTTTATGCTAAATCTGTAGAGTTTAGTAATGGCAAAAGATACCGTATG | |
| | GTCCACAAGGCCTAAAATAGCTACTATCTGTACCTTACAGAAAAAAAAAAAG | |
| | AGCTGACTCTTTTTCTGACCTCTGGTCTAAGTCAAGTCTGTCCCACAAGACAC | |
| | AAGGTACAATGCTTTTTCCACATGAGGTCAGGAAGAATCTCAAAACTACAGA | |
| | GTCAGGCCTCACATTATCAATAGGACCATCCTAGCCTTCTGCAAGCTTTCAGG | |
| | CTTTAGTCAAAGCTGGCACATTTTCAGTGAATCCTTAGTCCACTGGTGGTCTG | |
| | CAAGAGCCAGAGC | |
| JAK2- | GTAAGGGTGAAAATTCATTTGATGGAAATACTTGTGTATATTTAAAGACCCA | 328 |
| CT9 | ATTGCTCCTCTGGAGCTTGTACTTTCAAGAATGATTAATCTGTGTAATAAACT | |
| | GGTTACTACAGTCATTACATATAATTTTGTGTGAATAGGCTTTTTCATTTTTAA | |
| | GAAGTTTGTCTAGCTGAGATTAGTGGTGGATTTTCTCCCACTTCTGAAATGTT | |
| | CATTTATACTGGTTGCATTTTAAGATCATGAAACAATTCCAGTTACATTGTAA | |
| | AAAGGATATCTTACGAGTAATTTTATTGAACAAGTTAGAGGCATAAGCTTAA | |
| | GAGCATTTCCATGAAACAACACATGCAGCATTCCAGGAACTTG | |

Analysis of Demultiplexed Reads Obtained from High-Throughput Sequencing of NCN and TC Repeat Target Plasmids

A unique molecular identifier (UMI) was included within each target plasmid. The UMI served to distinguish reads that contained the unedited target sequence in the starting library from edited reads produced as a result of base editing (see Table 8). Seqkit package (grep)³⁷ was used to assign fastq files containing a given UMI to its starting NCN target plasmid. Batch analysis with CRISPResso2 was performed as described above for quantification of editing frequencies.

TABLE 8
Sequences of unique molecular identifiers associ-
ated with each target plasmid for NCN context
profiling and editing window profiling. For
sequence context profiling, each target plasmid
contains a target cytosine flanked by two
nucleotides of either A, T, C or G.

| | | SEQ | | No. of TC |
|---|---|---|---|---|
| | | ID | Sequence | repeats (top or |
| Plasmid | UMI | NO: | context | bottom) |
| pBM10a | TTTGTAGTCTAGTCT | 384 | ACA | |
| pBM10b | CATTATGATCGTACG | 385 | ACT | |
| pBM10c | CTATTCAGGGATTGA | 386 | ACC | |
| pBM10d | CTGATACCGGAAGAC | 387 | ACG | |
| pBM10e | ATCTCAGTTGAAGTG | 388 | TCA | |
| pBM10f | GTGTATACGACAGAG | 389 | TCT | |
| pBM10g | ACCGTGCACCTACCA | 390 | TCC | |
| pBM10h | AACCTCCTTAGTCTA | 391 | TCG | |
| pBM10i | AGTTCAGACCAATTG | 392 | CCA | |
| pBM10j | GTAGTTTGTCCAGAA | 393 | CCT | |
| pBM10k | CTCAGATTTTATCAC | 394 | CCC | |
| pBM10l | CAGAGGACGCACGCT | 395 | CCG | |
| pBM10m | CTACCTTTATGATCC | 396 | GCA | |
| pBM10n | TCCTTGGTCCTCGAG | 397 | GCT | |
| pBM10o | AAGAGGAGACGTCAG | 398 | GCC | |
| pBM10p | TCCAGATATCTTTAA | 399 | GCG | |
| pBM22a | TTTGTAGTCTAGTCT | 384 | | 12, top |
| pBM22b | CATTATGATCGTACG | 385 | | 13, top |
| pBM22c | CTATTCAGGGATTGA | 386 | | 14, top |
| pBM22d | CTGATACCGGAAGAC | 387 | | 15, top |
| pBM22e | ATCTCAGTTGAAGTG | 388 | | 16, top |
| pBM22f | GTGTATACGACAGAG | 389 | | 17, top |
| pBM22g | ACCGTGCACCTACCA | 390 | | 18, top |
| pBM23a | AACCTCCTTAGTCTA | 391 | | 12, bottom |
| pBM23b | AGTTCAGACCAATTG | 392 | | 13, bottom |
| pBM23c | GTAGTTTGTCCAGAA | 393 | | 14, bottom |
| pBM23d | CTCAGATTTTATCAC | 394 | | 15, bottom |
| pBM23e | CAGAGGACGCACGCT | 395 | | 16, bottom |
| pBM23f | CTACCTTTATGATCC | 396 | | 17, bottom |
| pBM23g | TCCTTGGTCCTCGAG | 397 | | 18, bottom |

### Bulk ATAC-Seq for Whole Mitochondrial Genome Sequencing

ATAC-seq was performed as previously described¹⁷. In brief, 5,000-10,000 cells were trypsinzed, washed with PBS, pelleted by centrifugation and lysed in 50 μL of lysis buffer (0.1% Igepal CA-360 (v/v %), 10 mM Tris-HCl, 10 mM NaCl and 3 mM MgCl₂ in nuclease-free water). Lysates were incubated on ice for 3 minutes, pelleted at 500 rcf for 10 minutes at 4° C. and tagmented with 2.5 μL of Tn5 transposase (Illumina #15027865) in a total volume of 10 μL containing 1×TD buffer (Illumina #15027866), 0.1% NP-40 (Sigma), and 0.3×PBS. Samples were incubated at 37° C. for 30 minutes on a thermomixer at 300 rpm. DNA was purified using the MinElute PCR Kit (Qiagen) and eluted in 10 μL elution buffer. All 10 μL of the eluate was amplified using indexed primers (1.25 μM each, sequences available as previously reported¹⁷) and NEBNext High-Fidelity 2×PCR Master Mix (New England Biolabs) in a total volume of 50 μL using the following protocol 72° C. for 5 min, 98° C. for 30 s, then 5 cycles of [98° C. for 10 s, 63° C. for 30 s, and 72° C. for 60 s], followed by a final 72° C. extension for 1 min. After the initial 5 cycles of pre-amplification, 5 μL of partially amplified library was used as input DNA in a total volume of 15 μL for quantitative PCR using SYBR Green to determine the number of additional cycles needed to reach ⅓ of the maximum fluorescence intensity. Typically, 3-8 cycles were conducted on the remaining 45 μL of partially amplified library. The final library was purified using a MinElute PCR kit (Qiagen) and quantified using a Qubit dsDNA HS Assay kit (Invitrogen) and a High Sensitivity DNA chip run on a Bioanalyzer 2100 system (Agilent). All libraries were sequenced using Nextseq High Output Cartridge kits on an Illumina Nextseq 500 sequencer. Libraries were sequenced using paired-end 2×75 cycles and demultiplexed using the bcl2fastq program. A sequencing depth of ˜3,000-8,000× was obtained per sample.

### Genome Sequencing and SNP Identification in Mitochondria

Analysis was performed as previously described¹⁷. Genome mapping was performed with BWA (v0.7.17) using NC_012920 genome as a reference. Duplicates were marked using Picard tools (v2.20.7). Pileup data from alignments were generated with SAMtools (v1.9) and variant calling was performed with VarScan2 (v2.4.3). Variants that were present at a frequency greater than 0.1% and a p-value less than 0.05 (Fisher's Exact Test) were called as high-confidence SNPs independently in each biological replicate. Only reads with Q>30 at a given position were taken into account when calling SNPs at that particular position. For FIGS. 33A-33F, all SNPs that were called in untreated samples were excluded from the analyses of treated samples. Each SNP was called in treated samples if it appeared in at least one biological replicate, and the average frequency was calculated by taking the average of all replicate(s) in which the SNP was present.

### Calculation of Average Off-Target C⋅G-to-T⋅a Editing Frequency

Analysis was performed as previously described^{17,38}. To calculate the mitochondrial genome-wide average off-target editing frequency for each DdCBE in FIG. 21I, REDItools was used (v1.2.1)³⁹. All nucleobases except cytosines and guanines were removed and the number of reads covering each C⋅G base pair with a PHRED quality score greater than 30 (Q>30) was calculated. The on-target C⋅G base pairs (depending on the DdCBE used in each treatment) were excluded in order to only consider off-target effects. C⋅G-to-T⋅A SNVs present at high frequencies (>50%) in both treated and untreated samples (that therefore did not arise from DdCBE treatment) were also excluded. The average off-target editing frequency was then calculated independently for each biological replicate of each treatment condition as: (number of reads in which a given C⋅G base pair was called as a T⋅A base pair, summed over all non-target C⋅G base pairs)+(total number of reads that covered all non-target C⋅G base pair).

### Oxygen Consumption Rate Analyses by Seahorse XF Analyzer

Seahorse plate was coated with 0.01% (w/v) poly-L-lysine (Sigma). 1.6×10⁴ cells were seeded on the coated Seahorse plate 16 hours prior to the analysis in the Seahorse XFe96 Analyzer (Agilent). Analysis was performed in the Seahorse XF DMEM Medium pH 7.4 (Agilent) supplemented with 10 mM glucose (Agilent), 2 mM L-glutamine (Gibco) and 1 mM sodium pyruvate (Gibco). Mito stress protocol was applied with the use of 1.5 mM oligomycin, 1 mM FCCP, and 1 mM piericidin+1 mM antimycin.

### Sequences

### TALE Sequences Used in DdCBEs

All right-side halves of DdCBEs have the general architecture of (from N- to C-terminus): COX8A MTS-3×FLAG-mitoTALE-2aa linker-DddA^{tox} half-4aa linker-1×-UGI-ATP5B 3′UTR.

All left-side halves of DdCBEs have the general architecture of (from N- to C-terminus): SOD2 MTS-3×HA-mitoTALE-2aa linker-DddAₜₒₓ half-4aa linker-1×-UGI-SOD2 3′UTR.

TALE sequences for SIRT6-DdCBE and JAK2-DdCBE are from Addgene plasmids #TAL2406, TAL2407, TAL2454 and TAL2455.

mitoTALE domains are annotated as: bold for N-terminal domain, underlined for RVD and bolded italics for C-terminal domain.

ND1-DdCBE Right mitoTALE repeat
(SEQ ID NO: 147)
DIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVH
AWRNALTGAPLNLTPEQVVAIASNNGGKQALETVQALLPVLCQAHGLTPQQVVAIASNIGGKQALE
TVQRLLPVLCQAHGLTPQQVVAIASNNGGKQALETVQRLLPVLCQAHGLTPEQVVAIASNGGGKQAL
ETVQALLPVLCQAHGLTPEQVVAIASNGGGKQALETVQALLPVLCQAHGLTPEQVVAIASNGGGKQA
LETVQALLPVLCQAHGLTPEQVVAIASNNGGKQALETVQRLLPVLCQAHGLTPEQVVAIASNIGGKQ
ALETVQALLPVLCQAHGLTPEQVVAIASNGGGKQALETVQRLLPVLCQAHGLTPEQVVAIASNNGGK
QALETVQALLPVLCQAHGLTPEQVVAIASHDGGKQALETVQALLPVLCQAHGLTPEQVVAIASNGGG
KQALETVQALLPVLCQAHGLTPEQVVAIASHDGGKQALETVQRLLPVLCQAHGLTPQQVVAIASNIG
GKQALETVQRLLPVLCQAHGLTPEQVVAIASHDGGKQALETVQALLPVLCQAHGLTPQQVVAIASHD
GGKQALETVQRLLPVLCQAHGLTPQQVVAIASHDGGKQALETVQRLLPVLCQAHGLTPQQVVAIASN
GGGRPALESIVAQLSRPDPALAALTNDHLVALACLGGRPALDAVKKGLG
ND1-DdCBE Left mitoTALE repeat
(SEQ ID NO: 149)
DIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVH
AWRNALTGAPLNLTPEQVVAIASHDGGKQALETVQALLPVLCQAHGLTPQQVVAIASNGGGKQALE
TVQRLLPVLCQAHGLTPQQVVAIASNIGGKQALETVQRLLPVLCQAHGLTPEQVVAIASNNGGKQAL
ETVQALLPVLCQAHGLTPEQVVAIASHDGGKQALETVQALLPVLCQAHGLTPEQVVAIASHDGGKQA
LETVQALLPVLCQAHGLTPEQVVAIASNGGGKQALETVQRLLPVLCQAHGLTPEQVVAIASNIGGKQ
ALETVQALLPVLCQAHGLTPEQVVAIASNNGGKQALETVQRLLPVLCQAHGLTPEQVVAIASHDGGK
QALETVQALLPVLCQAHGLTPEQVVAIASHDGGKQALETVQALLPVLCQAHGLTPEQVVAIASNNGG
KQALETVQALLPVLCQAHGLTPEQVVAIASNGGGKQALETVQRLLPVLCQAHGLTPQQVVAIASNGG
GKQALETVQRLLPVLCQAHGLTPQQVVAIASNGGGRPALESIVAQLSRPDPALAALTNDHLVALACLG
GRPALDAVKKGLG
ND1.2-DdCBE Left mitoTALE repeat
(SEQ ID NO: 186)
DIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVH
AWRNALTGAPLNLTPEQVVAIASNIGGKQALETVQALLPVLCQAHGLTPQQVVAIASNGGGKQALE
TVQRLLPVLCQAHGLTPQQVVAIASNIGGKQALETVQRLLPVLCQAHGLTPEQVVAIASNGGGKQAL
ETVQALLPVLCQAHGLTPEQVVAIASNIGGKQALETVQALLPVLCQAHGLTPEQVVAIASNNGGKQA
LETVQALLPVLCQAHGLTPEQVVAIASHDGGKQALETVQRLLPVLCQAHGLTPQQVVAIASHDGGKQ
ALETVQALLPVLCQAHGLTPEQVVAIASNGGGKQALETVQRLLPVLCQAHGLTPEQVVAIASNIGGK
QALETVQRLLPVLCQAHGLTPEQVVAIASNNGGKQALETVQALLPVLCQAHGLTPQQVVAIASNIGG
RPALESIVAQLSRPDPALAALTNDHLVALACLGGRPALDAVKKGLG
ND1.2-DdCBE Left mitoTALE repeat
(SEQ ID NO: 187)
DIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVH
AWRNALTGAPLNLTPEQVVAIASHDGGKQALETVQALLPVLCQAHGLTPQQVVAIASHDGGKQALE
TVQRLLPVLCQAHGLTPQQVVAIASNGGGKQALETVQRLLPVLCQAHGLTPEQVVAIASNIGGKQAL
ETVQALLPVLCQAHGLTPEQVVAIASNIGGKQALETVQALLPVLCQAHGLTPEQVVAIASNGGGKQA
LETVQALLPVLCQAHGLTPEQVVAIASNNGGKQALETVQRLLPVLCQAHGLTPQQVVAIASHDGGKQ
ALETVQALLPVLCQAHGLTPEQVVAIASNGGGKQALETVQRLLPVLCQAHGLTPQQVVAIASNGGGR
PALESIVAQLSRPDPALAALTNDHLVALACLGGRPALDAVKKGLG
ND2-DdCBE Right mitoTALE repeat
(SEQ ID NO: 151)
DIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVH
AWRNALTGAPLNLTPEQVVAIASNIGGKQALETVQALLPVLCQAHGLTPQQVVAIASNNGGKQALE
TVQRLLPVLCQAHGLTPQQVVAIASNIGGKQALETVQRLLPVLCQAHGLTPEQVVAIASNIGGKQALE
TVQALLPVLCQAHGLTPEQVVAIASHDGGKQALETVQALLPVLCQAHGLTPEQVVAIASNGGGKQAL
ETVQALLPVLCQAHGLTPEQVVAIASNNGGKQALETVQRLLPVLCQAHGLTPEQVVAIASHDGGKQA
LETVQALLPVLCQAHGLTPEQVVAIASNGGGKQALETVQRLLPVLCQAHGLTPEQVVAIASNIGGKQ
ALETVQALLPVLCQAHGLTPEQVVAIASNGGGKQALETVQALLPVLCQAHGLTPEQVVAIASNGGGK
QALETVQALLPVLCQAHGLTPEQVVAIASNIGGKQALETVQRLLPVLCQAHGLTPQQVVAIASNGGG
KQALETVQRLLPVLCQAHGLTPQQVVAIASNGGGRPALESIVAQLSRPDPALAALTNDHLVALACLGG
RPALDAVKKGLG
ND2-DdCBE Left mitoTALE repeat
(SEQ ID NO: 171)
DIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVH
AWRNALTGAPLNLTPEQVVAIASHDGGKQALETVQALLPVLCQAHGLTPQQVVAIASNGGGKQALE
TVQRLLPVLCQAHGLTPQQVVAIASNGGGKQALETVQRLLPVLCQAHGLTPEQVVAIASNIGGKQAL
ETVQALLPVLCQAHGLTPEQVVAIASNNGGKQALETVQALLPVLCQAHGLTPEQVVAIASHDGGKQA
LETVQALLPVLCQAHGLTPEQVVAIASNIGGKQALETVQRLLPVLCQAHGLTPEQVVAIASNGGGKQ
ALETVQALLPVLCQAHGLTPEQVVAIASNIGGKQALETVQRLLPVLCQAHGLTPEQVVAIASHDGGK
QALETVQALLPVLCQAHGLTPEQVVAIASNGGGKQALETVQALLPVLCQAHGLTPEQVVAIASHDGG
KQALETVQALLPVLCQAHGLTPEQVVAIASHDGGKQALETVQRLLPVLCQAHGLTPQQVVAIASNGG
GKQALETVQRLLPVLCQAHGLTPEQVVAIASHDGGKQALETVQALLPVLCQAHGLTPEQVVAIASNI
GGKQALETVQALLPVLCQAHGLTPEQVVAIASNIGGKQALETVQALLPVLCQAHGLTPQQVVAIASN
GGGRPALESIVAQLSRPDPALAALTNDHLVALACLGGRPALDAVKKGLG
ND4-DdCBE Right mitoTALE repeat
(SEQ ID NO: 154)
DIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVH
AWRNALTGAPLNLTPEQVVAIASHDGGKQALETVQALLPVLCQAHGLTPQQVVAIASHDGGKQALE
TVQRLLPVLCQAHGLTPQQVVAIASNGGGKQALETVQRLLPVLCQAHGLTPEQVVAIASNNGGKQAL
ETVQALLPVLCQAHGLTPEQVVAIASNGGGKQALETVQALLPVLCQAHGLTPEQVVAIASNIGGKQA
LETVQALLPVLCQAHGLTPEQVVAIASNIGGKQALETVQRLLPVLCQAHGLTPEQVVAIASNNGGKQ
ALETVQALLPVLCQAHGLTPEQVVAIASNGGGKQALETVQRLLPVLCQAHGLTPEQVVAIASNIGGK
QALETVQRLLPVLCQAHGLTPEQVVAIASNNGGKQALETVQALLPVLCQAHGLTPEQVVAIASNNGG
KQALETVQALLPVLCQAHGLTPEQVVAIASNIGGKQALETVQALLPVLCQAHGLTPEQVVAIASNNG
GKQALETVQRLLPVLCQAHGLTPQQVVAIASNIGGKQALETVQRLLPVLCQAHGLTPQQVVAIASNN
GGKQALETVQRLLPVLCQAHGLTPQQVVAIASNGGGRPALESIVAQLSRPDPALAALTNDHLVALACL
GGRPALDAVKKGLG
ND4-DdCBE Left mitoTALE repeat
(SEQ ID NO: 156)
DIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVH
AWRNALTGAPLNLTPEQVVAIASNNGGKQALETVQALLPVLCQAHGLTPQQVVAIASHDGGKQALE
TVQRLLPVLCQAHGLTPQQVVAIASNGGGKQALETVQRLLPVLCQAHGLTPEQVVAIASNIGGKQAL
ETVQALLPVLCQAHGLTPEQVVAIASNNGGKQALETVQALLPVLCQAHGLTPEQVVAIASNGGGKQA
LETVQALLPVLCQAHGLTPEQVVAIASNIGGKQALETVQRLLPVLCQAHGLTPEQVVAIASNIGGKQA
LETVQALLPVLCQAHGLTPEQVVAIASHDGGKQALETVQRLLPVLCQAHGLTPEQVVAIASHDGGKQ
ALETVQALLPVLCQAHGLTPEQVVAIASNIGGKQALETVQALLPVLCQAHGLTPEQVVAIASHDGGK
QALETVQALLPVLCQAHGLTPEQVVAIASNNGGKQALETVQRLLPVLCQAHGLTPQQVVAIASNGGG
KQALETVQRLLPVLCQAHGLTPEQVVAIASNGGGKQALETVQALLPVLCQAHGLTPEQVVAIASHDG
GKQALETVQALLPVLCQAHGLTPQQVVAIASNGGGRPALESIVAQLSRPDPALAALTNDHLVALACLG
GRPALDAVKKGLG
ND4.2-DdCBE Right mitoTALE repeat
(SEQ ID NO: 188)
DIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVH
AWRNALTGAPLNLTPEQVVAIASNNGGKQALETVQALLPVLCQAHGLTPQQVVAIASNNGGKQALE
TVQRLLPVLCQAHGLTPQQVVAIASNNGGKQALETVQRLLPVLCQAHGLTPEQVVAIASHDGGKQAL
ETVQALLPVLCQAHGLTPEQVVAIASNNGGKQALETVQALLPVLCQAHGLTPEQVVAIASNIGGKQA
LETVQALLPVLCQAHGLTPEQVVAIASNGGGKQALETVQRLLPVLCQAHGLTPEQVVAIASNGGGKQ
ALETVQALLPVLCQAHGLTPQQVVAIASNIGGKQALETVQRLLPVLCQAHGLTPEQVVAIASNGGGK
QALETVQRLLPVLCQAHGLTPQQVVAIASNNGGKQALETVQRLLPVLCQAHGLTPQQVVAIASNIGG
RPALESIVAQLSRPDPALAALTNDHLVALACLGGRPALDAVKKGLG
ND4.2-DdCBE Left mitoTALE repeat
(SEQ ID NO: 189)
DIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVH
AWRNALTGAPLNLTPEQVVAIASNNGGKQALETVQALLPVLCQAHGLTPQQVVAIASNIGGKQALE
TVQRLLPVLCQAHGLTPQQVVAIASNIGGKQALETVQRLLPVLCQAHGLTPEQVVAIASNNGGKQAL
ETVQALLPVLCQAHGLTPEQVVAIASHDGGKQALETVQALLPVLCQAHGLTPEQVVAIASNGGGKQA
LETVQALLPVLCQAHGLTPEQVVAIASNGGGKQALETVQRLLPVLCQAHGLTPEQVVAIASHDGGKQ
ALETVQALLPVLCQAHGLTPQQVVAIASNIGGKQALETVQRLLPVLCQAHGLTPQQVVAIASHDGGK
QALETVQRLLPVLCQAHGLTPQQVVAIASHDGGRPALESIVAQLSRPDPALAALTNDHLVALACLGGR
PALDAVKKGLG
ND4.3-DdCBE Right mitoTALE repeat
(SEQ ID NO: 190)
DIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVH
AWRNALTGAPLNLTPEQVVAIASNNGGKQALETVQALLPVLCQAHGLTPQQVVAIASNNGGKQALE
TVQRLLPVLCQAHGLTPQQVVAIASHDGGKQALETVQRLLPVLCQAHGLTPEQVVAIASNGGGKQAL
ETVQALLPVLCQAHGLTPEQVVAIASNNGGKQALETVQALLPVLCQAHGLTPEQVVAIASNGGGKQA
LETVQALLPVLCQAHGLTPEQVVAIASNGGGKQALETVQRLLPVLCQAHGLTPEQVVAIASNIGGKQ
ALETVQALLPVLCQAHGLTPEQVVAIASHDGGKQALETVQRLLPVLCQAHGLTPQQVVAIASNGGGR
PALESIVAQLSRPDPALAALTNDHLVALACLGGRPALDAVKKGLG
ND4.3-DdCBE Left mitoTALE repeat
(SEQ ID NO: 191)
DIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVH
AWRNALTGAPLNLTPEQVVAIASHDGGKQALETVQALLPVLCQAHGLTPQQVVAIASNGGGKQALE
TVQRLLPVLCQAHGLTPQQVVAIASNGGGKQALETVQRLLPVLCQAHGLTPEQVVAIASHDGGKQAL
ETVQALLPVLCQAHGLTPEQVVAIASNIGGKQALETVQALLPVLCQAHGLTPEQVVAIASNIGGKQAL
ETVQALLPVLCQAHGLTPEQVVAIASNGGGKQALETVQRLLPVLCQAHGLTPEQVVAIASHDGGKQA
LETVQALLPVLCQAHGLTPQQVVAIASNIGGKQALETVQRLLPVLCQAHGLTPQQVVAIASNNGGKQ
ALETVQRLLPVLCQAHGLTPEQVVAIASHDGGKQALETVQRLLPVLCQAHGLTPQQVVAIASHDGGR
PALESIVAQLSRPDPALAALTNDHLVALACLGGRPALDAVKKGLG
ND5.2-DdCBE Right mitoTALE repeat (Note: Terminal NG RVD recognizes a mismatched
T instead of a G in the reference genome)
(SEQ ID NO: 161)
DIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVH
AWRNALTGAPLNLTPQQVVAIASNNGGKQALETVQRLLPVLCQAHGLTPEQVVAIASHDGGKQALE
TVQRLLPVLCQAHGLTPQQVVAIASNGGGKQALETVQRLLPVLCQAHGLTPEQVVAIASNIGGKQAL
ETVQALLPVLCQAHGLTPQQVVAIASNNGGKQALETVQRLLPVLCQAHGLTPQQVVAIASNNGGKQ
ALETVQRLLPVLCQAHGLTPEQVVAIASHDGGKQALETVQRLLPVLCQAHGLTPQQVVAIASNGGGK
QALETVQRLLPVLCQAHGLTPQQVVAIASNNGGKQALETVQRLLPVLCQAHGLTPEQVVAIASHDGG
KQALETVQRLLPVLCQAHGLTPEQVVAIASHDGGKQALETVQRLLPVLCQAHGLTPEQVVAIASNIG
GKQALETVQALLPVLCQAHGLTPEQVVAIASNIGGKQALETVQALLPVLCQAHGLTPQQVVAIASNG
GGKQALETVQRLLPVLCQAHGLTPQQVVAIASNNGGKQALETVQRLLPVLCQAHGLTPQQVVAIASN
GGGRPALESIVAQLSRPDPALAALTNDHLVALACLGGRPALDAVKKGLG
ND5.2-DdCBE Right mitoTALE repeat (non-mismatched)
(SEQ ID NO: 192)
DIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVH
AWRNALTGAPLNLTPQQVVAIASNNGGKQALETVQRLLPVLCQAHGLTPEQVVAIASHDGGKQALE
TVQRLLPVLCQAHGLTPQQVVAIASNGGGKQALETVQRLLPVLCQAHGLTPEQVVAIASNIGGKQAL
ETVQALLPVLCQAHGLTPQQVVAIASNNGGKQALETVQRLLPVLCQAHGLTPQQVVAIASNNGGKQ
ALETVQRLLPVLCQAHGLTPEQVVAIASHDGGKQALETVQRLLPVLCQAHGLTPQQVVAIASNGGGK
QALETVQRLLPVLCQAHGLTPQQVVAIASNNGGKQALETVQRLLPVLCQAHGLTPEQVVAIASHDGG
KQALETVQRLLPVLCQAHGLTPEQVVAIASHDGGKQALETVQRLLPVLCQAHGLTPEQVVAIASNIG
GKQALETVQALLPVLCQAHGLTPEQVVAIASNIGGKQALETVQALLPVLCQAHGLTPQQVVAIASNG
GGKQALETVQRLLPVLCQAHGLTPQQVVAIASNNGGKQALETVQRLLPVLCQAHGLTPQQVVAIASN
NGGRPALESIVAQLSRPDPALAALTNDHLVALACLGGRPALDAVKKGLG
ND5.2-DdCBE Left mitoTALE repeat
(SEQ ID NO: 173)
DIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVH
AWRNALTGAPLNLTPEQVVAIASHDGGKQALETVQALLPVLCQAHGLTPQQVVAIASNIGGKQALE
TVQRLLPVLCQAHGLTPQQVVAIASNIGGKQALETVQRLLPVLCQAHGLTPEQVVAIASNIGGKQALE
TVQALLPVLCQAHGLTPEQVVAIASNIGGKQALETVQALLPVLCQAHGLTPEQVVAIASHDGGKQAL
ETVQALLPVLCQAHGLTPEQVVAIASHDGGKQALETVQRLLPVLCQAHGLTPEQVVAIASNIGGKQA
LETVQALLPVLCQAHGLTPEQVVAIASNGGGKQALETVQRLLPVLCQAHGLTPEQVVAIASNIGGKQ
ALETVQALLPVLCQAHGLTPEQVVAIASHDGGKQALETVQALLPVLCQAHGLTPEQVVAIASHDGGK
QALETVQALLPVLCQAHGLTPEQVVAIASNGGGKQALETVQRLLPVLCQAHGLTPQQVVAIASHDGG
KQALETVQRLLPVLCQAHGLTPQQVVAIASNGGGRPALESIVAQLSRPDPALAALTNDHLVALACLGG
RPALDAVKKGLG
ND5.4-DdCBE Right mitoTALE repeat
(SEQ ID NO: 193)
DIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVH
AWRNALTGAPLNLTPEQVVAIASNNGGKQALETVQALLPVLCQAHGLTPQQVVAIASHDGGKQALE
TVQRLLPVLCQAHGLTPQQVVAIASNGGGKQALETVQRLLPVLCQAHGLTPEQVVAIASNIGGKQAL
ETVQALLPVLCQAHGLTPEQVVAIASNNGGKQALETVQALLPVLCQAHGLTPEQVVAIASNNGGKQA
LETVQALLPVLCQAHGLTPEQVVAIASNGGGKQALETVQRLLPVLCQAHGLTPEQVVAIASNNGGKQ
ALETVQALLPVLCQAHGLTPQQVVAIASNGGGRPALESIVAQLSRPDPALAALTNDHLVALACLGGRP
ALDAVKKGLG
ND5.4-DdCBE Left mitoTALE repeat
(SEQ ID NO: 194)
DIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVH
AWRNALTGAPLNLTPEQVVAIASHDGGKQALETVQALLPVLCQAHGLTPQQVVAIASNIGGKQALE
TVQRLLPVLCQAHGLTPQQVVAIASNGGGKQALETVQRLLPVLCQAHGLTPEQVVAIASNIGGKQAL
ETVQALLPVLCQAHGLTPEQVVAIASNIGGKQALETVQALLPVLCQAHGLTPEQVVAIASNGGGKQA
LETVQALLPVLCQAHGLTPEQVVAIASNIGGKQALETVQRLLPVLCQAHGLTPEQVVAIASNNGGKQ
ALETVQALLPVLCQAHGLTPEQVVAIASNGGGKQALETVQRLLPVLCQAHGLTPEQVVAIASNGGGK
QALETVQALLPVLCQAHGLTPEQVVAIASNIGGKQALETVQALLPVLCQAHGLTPEQVVAIASHDGG
KQALETVQRLLPVLCQAHGLTPQQVVAIASNIGGKQALETVQRLLPVLCQAHGLTPQQVVAIASNIGG
RPALESIVAQLSRPDPALAALTNDHLVALACLGGRPALDAVKKGLG
ATP8-DdCBE Right mitoTALE repeat
(SEQ ID NO: 12)
DIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVH
AWRNALTGAPLNLTPQQVVAIASNIGGKQALETVQRLLPVLCQAHGLTPEQVVAIASNGGGKQALE
TVQRLLPVLCQAHGLTPQQVVAIASNNGGKQALETVQRLLPVLCQAHGLTPEQVVAIASNNGGKQAL
ETVQALLPVLCQAHGLTPQQVVAIASNNGGKQALETVQRLLPVLCQAHGLTPQQVVAIASHDGGKQ
ALETVQRLLPVLCQAHGLTPEQVVAIASNGGGKQALETVQRLLPVLCQAHGLTPQQVVAIASNGGGK
QALETVQRLLPVLCQAHGLTPQQVVAIASNGGGKQALETVQRLLPVLCQAHGLTPEQVVAIASNNGG
KQALETVQRLLPVLCQAHGLTPEQVVAIASNNGGKQALETVQRLLPVLCQAHGLTPQQVVAIASNGG
GRPALESIVAQLSRPDPALAALTNDHLVALACLGGRPALDAVKKGLG
ATP8-DdCBE Left mitoTALE repeat (Note: Terminal NG RVD recognizes a mismatched T
instead of a C in the reference genome)
(SEQ ID NO: 170)
DIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVH
AWRNALTGAPLNLTPEQVVAIASNIGGKQALETVQALLPVLCQAHGLTPQQVVAIASNGGGKQALE
TVQRLLPVLCQAHGLTPQQVVAIASNGGGKQALETVQRLLPVLCQAHGLTPEQVVAIASNIGGKQAL
ETVQALLPVLCQAHGLTPEQVVAIASNIGGKQALETVQALLPVLCQAHGLTPEQVVAIASNIGGKQAL
ETVQALLPVLCQAHGLTPEQVVAIASHDGGKQALETVQRLLPVLCQAHGLTPEQVVAIASNIGGKQA
LETVQALLPVLCQAHGLTPEQVVAIASHDGGKQALETVQRLLPVLCQAHGLTPEQVVAIASNIGGKQ
ALETVQALLPVLCQAHGLTPEQVVAIASNIGGKQALETVQALLPVLCQAHGLTPEQVVAIASNIGGKQ
ALETVQALLPVLCQAHGLTPEQVVAIASHDGGKQALETVQRLLPVLCQAHGLTPQQVVAIASNGGGK
QALETVQRLLPVLCQAHGLTPEQVVAIASNIGGKQALETVQALLPVLCQAHGLTPQQVVAIASNGGG
RPALESIVAQLSRPDPALAALTNDHLVALACLGGRPALDAVKKGLG
ATP8-DdCBE Left mitoTALE repeat (non-mismatched)
(SEQ ID NO: 195)
DIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVH
AWRNALTGAPLNLTPEQVVAIASNIGGKQALETVQALLPVLCQAHGLTPQQVVAIASNGGGKQALE
TVQRLLPVLCQAHGLTPQQVVAIASNGGGKQALETVQRLLPVLCQAHGLTPEQVVAIASNIGGKQAL
ETVQALLPVLCQAHGLTPEQVVAIASNIGGKQALETVQALLPVLCQAHGLTPEQVVAIASNIGGKQAL
ETVQALLPVLCQAHGLTPEQVVAIASHDGGKQALETVQRLLPVLCQAHGLTPEQVVAIASNIGGKQA
LETVQALLPVLCQAHGLTPEQVVAIASHDGGKQALETVQRLLPVLCQAHGLTPEQVVAIASNIGGKQ
ALETVQALLPVLCQAHGLTPEQVVAIASNIGGKQALETVQALLPVLCQAHGLTPEQVVAIASNIGGKQ
ALETVQALLPVLCQAHGLTPEQVVAIASHDGGKQALETVQRLLPVLCQAHGLTPQQVVAIASNGGGK
QALETVQRLLPVLCQAHGLTPEQVVAIASNIGGKQALETVQALLPVLCQAHGLTPQQVVAIASHDGG
RPALESIVAQLSRPDPALAALTNDHLVALACLGGRPALDAVKKGLG

### Sequences of Full-Length DddA Variants

DddA1 (T1380I)
(SEQ ID NO: 27)
GSYALGPYQISAPQLPAYNGQTVGTFYYVNDAGGLESKVFSSGGPTPYPNYANAGHVEGQSALFMRD
NGISEGLVFHNNPEGTCGFCVNMIETLLPENAKMTVVPPEGAIPVKRGATGETKVFTGNSNSPKSPTK
GGC
DddA2 (T1314A + T1380I)
(SEQ ID NO: 28)
GSYALGPYQISAPQLPAYNGQTVGAFYYVNDAGGLESKVFSSGGPTPYPNYANAGHVEGQSALFMR
DNGISEGLVFHNNPEGTCGFCVNMIETLLPENAKMTVVPPEGAIPVKRGATGETKVFTGNSNSPKSPT
KGGC
DddA3 (T1314A + T1380I + E1396K)
(SEQ ID NO: 31)
GSYALGPYQISAPQLPAYNGQTVGAFYYVNDAGGLESKVFSSGGPTPYPNYANAGHVEGQSALFMR
DNGISEGLVFHNNPEGTCGFCVNMIETLLPENAKMTVVPPKGAIPVKRGATGETKVFTGNSNSPKSPT
KGGC
DddA4 (T1380I + T1413I)
(SEQ ID NO: 30)
GSYALGPYQISAPQLPAYNGQTVGTFYYVNDAGGLESKVFSSGGPTPYPNYANAGHVEGQSALFMRD
NGISEGLVFHNNPEGTCGFCVNMIETLLPENAKMTVVPPEGAIPVKRGATGETKVFIGNSNSPKSPTKG
GC
DddA5 (Q1310R + S1330I + T1380I)
(SEQ ID NO: 29)
GSYALGPYQISAPQLPAYNGRTVGTFYYVNDAGGLESKVFISGGPTPYPNYANAGHVEGQSALFMRD
NGISEGLVFHNNPEGTCGFCVNMIETLLPENAKMTVVPPEGAIPVKRGATGETKVFTGNSNSPKSPTK
GGC
DddA6 (Q1310R + S1330I + T1380I + T1413I)
(SEQ ID NO: 45)
GSYALGPYQISAPQLPAYNGRTVGTFYYVNDAGGLESKVFISGGPTPYPNYANAGHVEGQSALFMRD
NGISEGLVFHNNPEGTCGFCVNMIETLLPENAKMTVVPPEGAIPVKRGATGETKVFIGNSNSPKSPTKG
GC
DddA7 (T1314A + G1344R + V1364M + E1370K + T1380I + T1413I)
(SEQ ID NO: 47)
GSYALGPYQISAPQLPAYNGQTVGAFYYVNDAGGLESKVFSSGGPTPYPNYANARHVEGQSALFMR
DNGISEGLMFHNNPKGTCGFCVNMIETLLPENAKMTVVPPEGAIPVKRGATGETKVFIGNSNSPKSPT
KGGC
DddA8 (N1342S + G1344R + V1364M + E1370K + T1380I + T1413I)
(SEQ ID NO: 48)
GSYALGPYQISAPQLPAYNGQTVGTFYYVNDAGGLESKVFSSGGPTPYPNYASARHVEGQSALFMRD
NGISEGLMFHNNPKGTCGFCVNMIETLLPENAKMTVVPPEGAIPVKRGATGETKVFIGNSNSPKSPTK
GGC
DddA9 (T1314A + G1344S + E1370K + T1380I + A1398T + T1413I)
(SEQ ID NO: 52)
GSYALGPYQISAPQLPAYNGQTVGAFYYVNDAGGLESKVFSSGGPTPYPNYANASHVEGQSALFMRD
NGISEGLVFHNNPKGTCGFCVNMIETLLPENAKMTVVPPEGTIPVKRGATGETKVFIGNSNSPKSPTKG
GC
DddA10 (T1314A + G1344S + E1370K + T1380I + E1396K + A1398T + T1413I)
(SEQ ID NO: 53)
GSYALGPYQISAPQLPAYNGQTVGAFYYVNDAGGLESKVFSSGGPTPYPNYANASHVEGQSALFMRD
NGISEGLVFHNNPKGTCGFCVNMIETLLPENAKMTVVPPKGTIPVKRGATGETKVFIGNSNSPKSPTK
GGC
DddA11 (S1330I + A1341V + N1342S + E1370K + T1380I + T1413I)
(SEQ ID NO: 54)
GSYALGPYQISAPQLPAYNGQTVGTFYYVNDAGGLESKVFISGGPTPYPNYVSAGHVEGQSALFMRD
NGISEGLVFHNNPKGTCGFCVNMIETLLPENAKMTVVPPEGAIPVKRGATGETKVFIGNSNSPKSPTK
GGC
DddA-7.9.1(E1325K + S1330I + A1341V + N1342S + E1370K + N1378S + T1380I +
T1413I)
(SEQ ID NO: 55)
GSYALGPYQISAPQLPAYNGQTVGTFYYVNDAGGLKSKVFISGGPTPYPNYVSAGHVEGQSALFMRD
NGISEGLVFHNNPKGTCGFCVSMIETLLPENAKMTVVPPEGAIPVKRGATGETKVFIGNSNSPKSPTKG
GC
DddA-7.12.1(S1330I + A1341I+ N1342S + E1370K + N1378S + T1380I + P1394S +
T1413I)
(SEQ ID NO: 56)
GSYALGPYQISAPQLPAYNGQTVGTFYYVNDAGGLESKVFISGGPTPYPNYISAGHVEGQSALFMRDN
GISEGLVFHNNPKGTCGFCVSMIETLLPENAKMTVVSPEGAIPVKRGATGETKVFIGNSNSPKSPTKGG
C
DddA-7.12.2 (S1330I + P1334S + A1341V+ N1342S + E1370K + N1378S + T1380I +
T1413I)
(SEQ ID NO: 57)
GSYALGPYQISAPQLPAYNGQTVGTFYYVNDAGGLESKVFISGGSTPYPNYVSAGHVEGQSALFMRD
NGISEGLVFHNNPKGTCGFCVSMIETLLPENAKMTVVPPEGAIPVKRGATGETKVFIGNSNSPKSPTKG
GC
DddA-7.12.3 (S1330I + P1334S + P1336S + A1341V+ N1342S + E1370K + N1378S +
T1380I + T1413I)
(SEQ ID NO: 58)
GSYALGPYQISAPQLPAYNGQTVGTFYYVNDAGGLESKVFISGGSTSYPNYVSAGHVEGQS
ALFMRDNGISEGLVFHNNPKGTCGFCVSMIETLLPENAKMTVVPPEGAIPVKRGATGETKVF
IGNSNSPKSPTKGGC

### Sequences Used in Fluorescence-Activated Cell Sorting of DdCBE Expressing Cells

Right-side halves of DdCBE have the general architecture of (from N- to C-terminus): COX8A MTS-3×FLAG-mitoTALE-GS linker-DddA half-SGGS linker-1×-UGI-GSG linker-P2A-eGFP-ATP5B 3′UTR

Right-side halves of DdCBE have the general architecture of (from N- to C-terminus): SOD2 MTS-3×HA-mitoTALE-GS linker-DddA half-SGGS linker-1×-UGI-GSG linker-P2A-mCherry-SOD2 3′UTR

P2A sequence
(SEQ ID NO: 223)
ATNFSLLKQAGDVEENPGP
eGFP sequence
(SEQ ID NO: 349)
MVSKGEELFTGVVPILVELDGDVNGHKFSVSGEGEGDATYGKLTLKFICT
TGKLPVPWPTLVTTLTYGVQCFSRYPDHMKQHDFFKSAMPEGYVQERTIF
FKDDGNYKTRAEVKFEGDTLVNRIELKGIDFKEDGNILGHKLEYNYNSHN
VYIMADKQKNGIKVNFKIRHNIEDGSVQLADHYQQNTPIGDGPVLLPDNH
YLSTQSALSKDPNEKRDHMVLLEFVTAAGITLGMDELYK
mCherry sequence
(SEQ ID NO: 350)
MVSKGEEDNMAIIKEFMRFKVHMEGSVNGHEFEIEGEGEGRPYEGTQTAK
LKVTKGGPLPFAWDILSPQFMYGSKAYVKHPADIPDYLKLSFPEGFKWER
VMNFEDGGVVTVTQDSSLQDGEFIYKVKLRGTNFPSDGPVMQKKTMGWEA
SSERMYPEDGALKGEIKQRLKLKDGGHYDAEVKTTYKAKKPVQLPGAYNV
NIKLDITSHNEDYTIVEQYERAEGRHSTGGMDELYK

### REFERENCES USED IN EXAMPLE 2

1. Brüser, C., Keller-Findeisen, J. & Jakobs, S. The TFAM-to-mtDNA ratio defines inner-cellular nucleoid populations with distinct activity levels. Cell Rep 37, 110000, doi:10.1016/j.celrep.2021.110000 (2021).
2. Kukat, C. et al. Super-resolution microscopy reveals that mammalian mitochondrial nucleoids have a uniform size and frequently contain a single copy of mtDNA. Proc Natl Acad Sci USA 108, 13534-13539, doi:10.1073/pnas.1109263108 (2011).
3. Robin, E. D. & Wong, R. Mitochondrial DNA molecules and virtual number of mitochondria per cell in mammalian cells. J Cell Physiol 136, 507-513, doi:10.1002/jcp.1041360316 (1988).
4. Yuan, Y. et al. Comprehensive molecular characterization of mitochondrial genomes in human cancers. Nature Genetics 52, 342-352, doi:10.1038/s41588-019-0557-x (2020).
5. Schapira, A. H. Mitochondrial diseases. Lancet 379, 1825-1834, doi:10.1016/s0140-6736(11)61305-6 (2012).
6. Vafai, S. B. & Mootha, V. K. Mitochondrial disorders as windows into an ancient organelle. Nature 491, 374-383, doi:10.1038/nature11707 (2012).
7. Stewart, J. B. & Chinnery, P. F. Extreme heterogeneity of human mitochondrial DNA from organelles to populations. Nature Reviews Genetics 22, 106-118, doi:10.1038/s41576-020-00284-x (2021).
8. Bacman, S. R., Williams, S. L., Pinto, M., Peralta, S. & Moraes, C. T. Specific elimination of mutant mitochondrial genomes in patient-derived cells by mitoTALENs. Nat Med 19, 1111-1113, doi:10.1038/nm.3261 (2013).
9. Gammage, P. A., Rorbach, J., Vincent, A. I., Rebar, E. J. & Minczuk, M. Mitochondrially targeted ZFNs for selective degradation of pathogenic mitochondrial genomes bearing large-scale deletions or point mutations. EMBO Mol Med 6, 458-466, doi:10.1002/emmm.201303672 (2014).
10. Peeva, V. et al. Linear mitochondrial DNA is rapidly degraded by components of the replication machinery. Nat Commun 9, 1727, doi:10.1038/s41467-018-04131-w (2018).
11. Nissanka, N., Bacman, S. R., Plastini, M. J. & Moraes, C. T. The mitochondrial DNA polymerase gamma degrades linear DNA fragments precluding the formation of deletions. Nat Commun 9, 2491, doi:10.1038/s41467-018-04895-1 (2018).
12. Jackson, C. B., Turnbull, D. M., Minczuk, M. & Gammage, P. A. Therapeutic Manipulation of mtDNA Heteroplasmy: A Shifting Perspective. Trends Mol Med 26, 698-709, doi:10.1016/j.molmed.2020.02.006 (2020).
13. Komor, A. C., Kim, Y. B., Packer, M. S., Zuris, J. A. & Liu, D. R. Programmable editing of a target base in genomic DNA without double-stranded DNA cleavage. Nature 533, 420, doi:10.1038/nature17946
14. Gaudelli, N. M. et al. Programmable base editing of A⋅T to G⋅C in genomic DNA without DNA cleavage. Nature 551, 464, doi:10.1038/nature24644 nature.com/articles/nature24644 #supplementary-information (2017).
15. Anzalone, A. V. et al. Search-and-replace genome editing without double-strand breaks or donor DNA. Nature, doi:10.1038/s41586-019-1711-4 (2019).
16. Gammage, P. A., Moraes, C. T. & Minczuk, M. Mitochondrial Genome Engineering: The Revolution May Not Be CRISPR-Ized. Trends Genet 34, 101-110, doi:10.1016/j.tig.2017.11.001 (2018).
17. Mok, B. Y. et al. A bacterial cytidine deaminase toxin enables CRISPR-free mitochondrial base editing. Nature 583, 631-637, doi:10.1038/s41586-020-2477-4 (2020).
18. Lee, H. et al. Mitochondrial DNA editing in mice with DddA-TALE fusion deaminases. Nat Commun 12, 1190, doi:10.1038/s41467-021-21464-1 (2021).
19. Kang, B. C. et al. Chloroplast and mitochondrial DNA editing in plants. Nat Plants 7, 899-905, doi:10.1038/s41477-021-00943-9 (2021).
20. Guo, J. et al. Precision modeling of mitochondrial diseases in zebrafish via DdCBE-mediated mtDNA base editing. Cell Discovery 7, 78, doi:10.1038/s41421-021-00307-9 (2021).
21. Thuronyi, B. W. et al. Continuous evolution of base editors with expanded target compatibility and improved activity. Nat Biotechnol 37, 1070-1079, doi:10.1038/s41587-019-0193-0 (2019).
22. Roth, T. B., Woolston, B. M., Stephanopoulos, G. & Liu, D. R. Phage-Assisted Evolution of Bacillus methanolicus Methanol Dehydrogenase 2. ACS Synthetic Biology 8, 796-806, doi:10.1021/acssynbio.8b00481 (2019).
23. Esvelt, K. M., Carlson, J. C. & Liu, D. R. A system for the continuous directed evolution of biomolecules. Nature 472, 499-503, doi:10.1038/nature09929 (2011).
24. Badran, A. H. & Liu, D. R. Development of potent in vivo mutagenesis plasmids with broad mutational spectra. Nat Commun 6, 8425, doi:10.1038/ncomms9425 (2015).
25. Carlson, J. C., Badran, A. H., Guggiana-Nilo, D. A. & Liu, D. R. Negative selection and stringency modulation in phage-assisted continuous evolution. Nature Chemical Biology 10, 216-222, doi:10.1038/nchembio.1453 (2014).
26. Szymczak, A. L. et al. Correction of multi-gene deficiency in vivo using a single ‘self-cleaving’ 2A peptide-based retroviral vector. Nature Biotechnology 22, 589-594, doi:10.1038/nbt957 (2004).
27. Reyon, D. et al. FLASH assembly of TALENs for high-throughput genome editing. Nature Biotechnology 30, 460-465, doi:10.1038/nbt.2170 (2012).
28. Fine, E. J., Cradick, T. J., Zhao, C. L., Lin, Y. & Bao, G. An online bioinformatics tool predicts zinc finger and TALE nuclease off-target cleavage. Nucleic Acids Research 42, e42-e42, doi:10.1093/nar/gkt1326 (2013).
29. Cuculis, L., Abil, Z., Zhao, H. & Schroeder, C. M. Direct observation of TALE protein dynamics reveals a two-state search mechanism. Nature Communications 6, 7277, doi:10.1038/ncomms8277 (2015).
30. Streubel, J., Blucher, C., Landgraf, A. & Boch, J. TAL effector RVD specificities and efficiencies. Nature Biotechnology 30, 593-595, doi:10.1038/nbt.2304 (2012).
31. Liao, Z. et al. The ND4 G11696A mutation may influence the phenotypic manifestation of the deafness-associated 12S rRNA A1555G mutation in a four-generation Chinese family. Biochem Bioph Res Co 362, 670-676, doi:https://doi.org/10.1016/j.bbrc.2007.08.034 (2007).
32. Gopal, R. K. et al. Early loss of mitochondrial complex I and rewiring of glutathione metabolism in renal oncocytoma. Proc Natl Acad Sci USA 115, E6283-E6290, doi:10.1073/pnas.1711888115 (2018).
33. Gehrke, J. M. et al. An APOBEC3A-Cas9 base editor with minimized bystander and off-target activities. Nat Biotechnol 36, 977-982, doi:10.1038/nbt.4199 (2018).
34. Hubbard, B. P. et al. Continuous directed evolution of DNA-binding proteins to improve TALEN specificity. Nat Methods 12, 939-942, doi:10.1038/nmeth.3515 (2015).
35. Badran, A. H. et al. Continuous evolution of Bacillus thuringiensis toxins overcomes insect resistance. Nature 533, 58-63, doi:10.1038/nature17938 (2016).
36. Clement, K. et al. CRISPResso2 provides accurate and rapid genome editing sequence analysis. Nat Biotechnol 37, 224-226, doi:10.1038/s41587-019-0032-3 (2019).
37. Shen, W., Le, S., Li, Y. & Hu, F. SeqKit: A Cross-Platform and Ultrafast Toolkit for FASTA/Q File Manipulation. PLOS ONE 11, e0163962, doi:10.1371/journal.pone.0163962 (2016).
38. Doman, J. L., Raguram, A., Newby, G. A. & Liu, D. R. Evaluation and minimization of Cas9-independent off-target DNA editing by cytosine base editors. Nature Biotechnology, doi:10.1038/s41587-020-0414-6 (2020).
39. Diroma, M. A., Ciaccia, L., Pesole, G. & Picardi, E. Elucidating the editome: bioinformatics approaches for RNA editing detection. Brief Bioinform 20, 436-447, doi:10.1093/bib/bbx129 (2019).
40. Yu, Q. et al. Single-strand specificity of APOBEC3G accounts for minus-strand deamination of the HIV genome. Nat Struct Mol Biol 11, 435-442, doi:10.1038/nsmb758 (2004).
41. Maiti, A. et al. Crystal structure of the catalytic domain of HIV-1 restriction factor APOBEC3G in complex with ssDNA. Nature Communications 9, 2460, doi:10.1038/s41467-018-04872-8 (2018).
42. Rathore, A. et al. The Local Dinucleotide Preference of APOBEC3G Can Be Altered from 5′-CC to 5′-TC by a Single Amino Acid Substitution. Journal of Molecular Biology 425, 4442-4454, doi:https://doi.org/10.1016/j.jmb.2013.07.040 (2013).
43. Fine, E. J., Cradick, T. J., Zhao, C. L., Lin, Y. & Bao, G. An online bioinformatics tool predicts zinc finger and TALE nuclease off-target cleavage. Nucleic Acids Research 42, e42-e42, doi:10.1093/nar/gkt1326 (2013).
44. Bacman, S. R. et al. MitoTALEN reduces mutant mtDNA load and restores tRNA(Ala) levels in a mouse model of heteroplasmic mtDNA mutation. Nat Med 24, 1696-1700, doi:10.1038/s41591-018-0166-8 (2018).
45. Mok, B. Y. et al. A bacterial cytidine deaminase toxin enables CRISPR-free mitochondrial base editing. Nature 583, 631-637, doi:10.1038/s41586-020-2477-4 (2020).

TABLE 9
List of predicted off-target nuclear DNA sites for SIRT6-DdCBE and JAK2-DdCBE

| | | | | | | | | | | | | | | | |
|---|---|---|---|---|---|---|---|---|---|---|---|---|---|---|---|
| | | | Nuclease | | | Left | | Right | Chromo- | Chromo- | | | | | Forward |
| | Homology | RVD | Orienta- | Spacer | Mis- | Half | Spacer | Half | some | some | Genomic | Closest | Primer | Forward | Melt |
| Ranking | Score | Score | tion | Length | matches | Site | Seq | Site | Name | Location | Region | Gene | Index | Primer | Temp |

JAK2-DdCBE

| | | | | | | | | | | | | | | | |
|---|---|---|---|---|---|---|---|---|---|---|---|---|---|---|---|
| 1 | 13 | 2.818 | L-20-R | 20 | 5_4 | gCTaA | atagaca | AGGTA | chr18 | 61057152 | Exon | VPS4B | 018- | GTGTG | 60.908 |
| | | | | | | AAAAG | attttctt | AAGAC | | | | | 061056920 | GTCAG | |
| | | | | | | ACaCT | cttta | AaTgtCt | | | | | | GACAA | |
| | | | | | | GaA | (SEQ | a (SEQ | | | | | | ACTGT | |
| | | | | | | (SEQ | ID NO: | ID NO: | | | | | | TCCC | |
| | | | | | | ID NO: | 571) | 671) | | | | | | (SEQ ID | |
| | | | | | | 423) | | | | | | | | NO: | |
| | | | | | | | | | | | | | | 722) | |
| 2 | 10 | 2.827 | L-12-R | 12 | 6_4 | TaaaA | ctataatgt | AaGTA | chr5 | 93680784 | Intron | KIAA0825 | 005- | GGGGC | 61.508 |
| | | | | | | AAAAa | tag | AAGAtA | | | | | 093680605 | TGGGA | |
| | | | | | | AgTCT | (SEQ ID | GTAGtA | | | | | | GAAGA | |
| | | | | | | GgA | NO: | a (SEQ | | | | | | GAGGA | |
| | | | | | | (SEQ | 572) | ID NO: | | | | | | A (SEQ | |
| | | | | | | ID NO: | | 522) | | | | | | ID NO: | |
| | | | | | | 424) | | | | | | | | 723) | |
| 3 | 26 | 2.834 | L-11-R | 11 | 6_2 | TCTGA | cacaggc | AGGaA | chr2 | 146083472 | Intergenic | DKFZp686O1327 | 002- | GTCCA | 59.148 |
| | | | | | | gCTCT | ctgc | AAGAg | | | | | 146083255 | CTCTG | |
| | | | | | | AAAAa | (SEQ ID | AGaAG | | | | | | TGTTA | |
| | | | | | | GCA | NO: | gca | | | | | | CCCAG | |
| | | | | | | (SEQ | 573) | (SEQ ID | | | | | | ATC | |
| | | | | | | ID NO: | | NO: | | | | | | (SEQ ID | |
| | | | | | | 425) | | 523) | | | | | | NO: | |
| | | | | | | | | | | | | | | 724) | |
| 4 | 20 | 2.836 | L-17-R | 17 | 5_3 | TCctAA | cgcatgta | AGcTAA | chr9 | 7420649 | Intergenic | TMEM2 | 009- | GCCGG | 61.088 |
| | | | | | | AAAGA | gctaggg | gGtCAG | | | | | 074206275 | TTTAG | |
| | | | | | | CTCTc | aa (SEQ | TgGCAg | | | | | | GTATG | |
| | | | | | | CA | ID NO: | (SEQ ID | | | | | | GGAAC | |
| | | | | | | (SEQ | 574) | NO: 524) | | | | | | CAC | |
| | | | | | | ID NO: | | | | | | | | (SEQ ID | |
| | | | | | | 426) | | | | | | | | NO: 725) | |
| 5 | 5 | 2.848 | R-11-L | 11 | 6_5 | tTaCTt | gatgatac | aGtgGA | chr10 | 119782471 | Intron | FIP2RAB11 | 010- | TTACC | 61.508 |
| | | | | | | aTaTC | att (SEQ | GTgTTT | | | | | 119782198 | CCTGG | |
| | | | | | | TaTAC | ID NO: | TTCAGt | | | | | | ATCCC | |
| | | | | | | CT | 575) | (SEQ ID | | | | | | CTCCC | |
| | | | | | | (SEQ | | NO: 525) | | | | | | T (SEQ | |
| | | | | | | ID NO: | | | | | | | | ID NO: | |
| | | | | | | 427) | | | | | | | | 726) | |
| 6 | 18 | 2.854 | R-17-L | 17 | 4_4 | cTGCTt | tttttttttt | TGCAG | chr11 | 12572661 | Intergenic | PARVA | 011- | GGTGT | 61.508 |
| | | | | | | CTcTC | tttttga | AGTCT | | | | | 012572487 | GAATC | |
| | | | | | | TTTACt | (SEQ ID | TgcTCt | | | | | | CGGGA | |
| | | | | | | T (SEQ | NO: | Gt (SEQ | | | | | | GGCAG | |
| | | | | | | ID NO: | 576) | ID NO: | | | | | | A (SEQ | |
| | | | | | | 428) | | 526) | | | | | | ID NO: | |
| | | | | | | | | | | | | | | 727) | |
| 7 | 5 | 2.86 | R-11-L | 11 | 6_5 | AaaaT | gctatccc | TGttGtG | chr4 | 176196444 | Intergenic | ADAM29 | 004- | GGAAC | 60.298 |
| | | | | | | ACTaT | atg | TtcTTTT | | | | | 176196333 | AGTGA | |
| | | | | | | CTcTA | (SEQ ID | tAGA | | | | | | TCAGT | |
| | | | | | | CCT | NO: | (SEQ ID | | | | | | TCTGT | |
| | | | | | | (SEQ | 577) | NO: | | | | | | TACCT | |
| | | | | | | ID NO: | | 527) | | | | | | GAC | |
| | | | | | | 429) | | | | | | | | (SEQ ID | |
| | | | | | | | | | | | | | | NO: | |
| | | | | | | | | | | | | | | 728) | |
| 8 | 10 | 2.876 | L-10-R | 10 | 6_4 | TCTcA | ttttaaaga | AGGTA | chr6 | 43313083 | Intron | ZNF318 | 006- | GCTAC | 61.508 |
| | | | | | | AAcca | g (SEQ | AAGAa | | | | | 043312871 | TGGGG | |
| | | | | | | ACTaT | ID NO: | AGaAG | | | | | | AGCCT | |
| | | | | | | aCA | 578) | aAa | | | | | | GAAGT | |
| | | | | | | (SEQ | | (SEQ ID | | | | | | G (SEQ | |
| | | | | | | ID NO: | | NO: | | | | | | ID NO: | |
| | | | | | | 430) | | 528) | | | | | | 729) | |
| 9 | 17 | 2.878 | L-12-R | 12 | 6_3 | TCTcA | aaacaaa | AGagAA | chr3 | 8587815 | Intron | LMCD1 | 003- | TCCGG | 61.508 |
| | | | | | | AAAAa | aaaaa | AGtCAG | | | | | 008587704 | ATTCT | |
| | | | | | | ACaaa | (SEQ ID | TAGCA | | | | | | CCTGC | |
| | | | | | | aCA | NO: 579) | T (SEQ | | | | | | TGAGG | |
| | | | | | | (SEQ | | ID NO: | | | | | | C (SEQ | |
| | | | | | | ID NO: | | 529) | | | | | | ID NO: | |
| | | | | | | 431) | | | | | | | | 730) | |
| 10 | 18 | 2.882 | R-17-L | 17 | 4_4 | ATaCT | attcctttg | TGtAGA | chr5 | 66297316 | Intron | MAST4 | 005- | CCGAC | 59.508 |
| | | | | | | caTGT | agaatttc | GgCTTT | | | | | 066297056 | TGCTC | |
| | | | | | | CTTTA | (SEQ ID | cTCAaA | | | | | | TGCCT | |
| | | | | | | CCc (SEQ | NO: 580) | (SEQ ID | | | | | | TCTGA | |
| | | | | | | ID NO: | | NO: 530) | | | | | | (SEQ ID | |
| | | | | | | 432) | | | | | | | | NO: | |
| | | | | | | | | | | | | | | 731) | |
| 11 | 18 | 2.884 | R-17-L | 17 | 4_4 | taGCT | tacagaa | aGCtGA | chr13 | 83643339 | Intergenic | SLITRK1 | 013- | GGCTA | 59.508 |
| | | | | | | ACTaT | aaaaaaa | cTCTTT | | | | | 083643051 | GGGGA | |
| | | | | | | CTgTA | aag | TTCtGA | | | | | | ACTCG | |
| | | | | | | CCT (SEQ | (SEQ ID | (SEQ ID | | | | | | ATGTC | |
| | | | | | | ID NO: | NO: | NO: | | | | | | (SEQ ID | |
| | | | | | | 433) | 581) | 531) | | | | | | NO: | |
| | | | | | | | | | | | | | | 732) | |
| 12 | 8 | 2.888 | R-11-L | 11 | 5_5 | tTaCTA | ataattttgt | TGaAtA | chr5 | 139007256 | Exon | UBE2D2 | 005- | GTGAT | 59.148 |
| | | | | | | CaGTC | g (SEQ | GgCTTT | | | | | 139006977 | GGGCT | |
| | | | | | | aTTAC | ID NO: | TTCAtt | | | | | | TTGTT | |
| | | | | | | aT (SEQ | 582) | (SEQ ID | | | | | | CCCAA | |
| | | | | | | ID NO: | | NO: 532) | | | | | | CTC | |
| | | | | | | 434) | | | | | | | | (SEQ ID | |
| | | | | | | | | | | | | | | NO: | |
| | | | | | | | | | | | | | | 733) | |
| 13 | 5 | 2.895 | R-17-L | 17 | 6_5 | tTcCTc | atcagttat | TttAaAG | chr1 | 235172003 | Intergenic | TOMM20 | 001- | CAAGA | 60.908 |
| | | | | | | CTtTC | aatcattt | TtTTTT | | | | | 235171809 | TAGGG | |
| | | | | | | TTaAC | (SEQ ID | TtAGA | | | | | | CCTTG | |
| | | | | | | aT | NO: 583) | (SEQ ID | | | | | | CTGTG | |
| | | | | | | (SEQ | | NO: 533) | | | | | | TTGC | |
| | | | | | | ID NO: | | | | | | | | (SEQ ID | |
| | | | | | | 435) | | | | | | | | NO: 734) | |
| 14 | 13 | 2.895 | R-18-L | 18 | 5_4 | cTcCac | tgcgcac | gGaAG | chr2 | 5508616 | Intergenic | SOX11 | 002- | CCTGG | 61.088 |
| | | | | | | CTGTC | acactgttt | AtgCTT | | | | | 005508374 | GATGG | |
| | | | | | | TTTAC | gg (SEQ | TTTCtG | | | | | | GAATC | |
| | | | | | | CT (SEQ | ID NO: | A (SEQ | | | | | | AGAAC | |
| | | | | | | ID NO: | 584) | ID NO: | | | | | | TGC | |
| | | | | | | 436) | | 534) | | | | | | (SEQ ID | |
| | | | | | | | | | | | | | | NO: 735) | |
| 15 | 17 | 2.907 | L-10-R | 10 | 6_3 | TCTGA | gttataga | AtGTAg | chrX | 87145708 | Intergenic | KLHL4 | 00X- | CAGGT | 59.038 |
| | | | | | | AAAAG | aa (SEQ | AGACA | | | | | 087145430 | ATTAT | |
| | | | | | | ACatTc | ID NO: | GggGgA | | | | | | GTTAG | |
| | | | | | | CA (SEQ | 585) | a (SEQ | | | | | | GCCAT | |
| | | | | | | ID NO: | | ID NO: | | | | | | GGGG | |
| | | | | | | 437) | | 535) | | | | | | (SEQ ID | |
| | | | | | | | | | | | | | | NO: 736) | |
| 16 | 5 | 2.915 | L-20-R | 20 | 6_5 | gCTaA | agttgcttt | AGGgA | chr15 | 72509552 | Intron | PKM | 015- | CGTTC | 61.288 |
| | | | | | | AAgAG | gtcagga | AgGAgA | | | | | 072509312 | TCATG | |
| | | | | | | ACTCa | caag | GTAGg | | | | | | GACCT | |
| | | | | | | GCc (SEQ | (SEQ ID | ga (SEQ | | | | | | GCCCA | |
| | | | | | | ID NO: | NO: | ID NO: | | | | | | GT | |
| | | | | | | 438) | 586) | 536) | | | | | | (SEQ ID | |
| | | | | | | | | | | | | | | NO: | |
| | | | | | | | | | | | | | | 737) | |
| 17 | 13 | 2.922 | R-10-L | 10 | 5 4 | ATcCTt | caccttca | TGaAG | chr14 | 99037124 | Intergenic | C14orf177 | 014- | CTATC | 59.848 |
| | | | | | | CTGTC | tt (SEQ | AGTaTT | | | | | 099036828 | AGTGA | |
| | | | | | | TTTAat | ID NO: | TTTtAtg | | | | | | AATCTT | |
| | | | | | | T (SEQ | 587) | (SEQ ID | | | | | | TGCCC | |
| | | | | | | ID NO: | | NO: | | | | | | ATTCT | |
| | | | | | | 439) | | 537) | | | | | | GTTTG | |
| | | | | | | | | | | | | | | C (SEQ | |
| | | | | | | | | | | | | | | ID NO: | |
| | | | | | | | | | | | | | | 738) | |
| 18 | 10 | 2.926 | L-18-R | 18 | 6_4 | gCTaA | ttattgaaa | AGGTA | chr12 | 52849222 | Intergenic | KRT6B | 012- | GGGCT | 59.258 |
| | | | | | | AAcAa | atttccaa | AAGAg | | | | | 052848936 | TCCCT | |
| | | | | | | ACTtT | c (SEQ | AacAGt | | | | | | TTCTTC | |
| | | | | | | GtA | ID NO: | AT | | | | | | CACTA | |
| | | | | | | (SEQ | 588) | (SEQ ID | | | | | | G (SEQ | |
| | | | | | | ID NO: | | NO: | | | | | | ID NO: | |
| | | | | | | 440) | | 538) | | | | | | 739) | |
| 19 | 10 | 2.926 | R-18-L | 18 | 6_4 | tTtaTA | cctactgg | TtCtGA | chr2 | 80045175 | Intron | CTNNA2 | 002- | CCCCT | 61.508 |
| | | | | | | CaGTg | gtgtttcttt | GTCTT | | | | | 080045032 | CTCCC | |
| | | | | | | TTTAa | (SEQ ID | TTTtAG | | | | | | AAAAG | |
| | | | | | | CT | NO: 589) | c (SEQ | | | | | | GAGAG | |
| | | | | | | (SEQ | | ID NO: | | | | | | C (SEQ | |
| | | | | | | ID NO: | | 539) | | | | | | ID NO: | |
| | | | | | | 441) | | | | | | | | 740) | |
| 20 | 5 | 2.926 | L-19-R | 19 | 6_5 | aCTctA | gtgctttgc | tGGaAA | chr9 | 87248678 | Intergenic | NTRK2 | 009- | GCAAG | 59.258 |
| | | | | | | AAAaA | agtttatttt | AGAgA | | | | | 408728380 | TGATA | |
| | | | | | | CTCTta | (SEQ ID | GTAGg | | | | | | CAACC | |
| | | | | | | A (SEQ | NO: | Ag | | | | | | CCAAG | |
| | | | | | | ID NO: | 590) | (SEQ ID | | | | | | CC | |
| | | | | | | 442) | | NO: | | | | | | (SEQ ID | |
| | | | | | | | | 540) | | | | | | NO: 741) | |
| 21 | 13 | 2.932 | R-11-L | 11 | 5_4 | AcGCT | gcattatc | TGCAatt | chr5 | 160334359 | Intergenic | LCC285629 | 005- | CCAGC | 61.288 |
| | | | | | | AaacT | cta | TtTTTT | | | | | 160334092 | CTTCA | |
| | | | | | | CTTTA | (SEQ ID | (SEQ ID | | | | | | GTTTG | |
| | | | | | | CCT | NO: 591) | NO: 541) | | | | | | CCAAG | |
| | | | | | | (SEQ | | TgAGA | | | | | | GG | |
| | | | | | | ID NO: | | | | | | | | (SEQ ID | |
| | | | | | | 443) | | | | | | | | NO: 742) | |
| 22 | 10 | 2.934 | R-20-L | 20 | 6_4 | taaCTA | cttagaga | TGtcaA | chr3 | 99365326 | Intron | MIR548G | 003- | GCACA | 61.288 |
| | | | | | | CctTCT | catgtgag | GTaTTT | | | | | 65077 | CCAGA | |
| | | | | | | TTACC | gtcc | TTCAG | | | | | | GGAGA | |
| | | | | | | a (SEQ | (SEQ ID | A (SEQ | | | | | | GCTGC | |
| | | | | | | ID NO: | NO: | ID NO: | | | | | | TT | |
| | | | | | | 444) | 592) | 542) | | | | | | (SEQ ID | |
| | | | | | | | | | | | | | | NO: | |
| | | | | | | | | | | | | | | 743) | |
| 23 | 10 | 2.937 | R-18-L | 18 | 6_4 | cTaCTt | tttcacttat | TcttcAG | chr14 | 38009449 | Intron | MIPQL1 | 014- | CCCAG | 61.508 |
| | | | | | | CTacC | gaattcat | TCTTTT | | | | | 038009333 | TCTTG | |
| | | | | | | TTcAC | (SEQ ID | TCAGA | | | | | | GGCAG | |
| | | | | | | CT | NO: 593) | (SEQ ID | | | | | | CCCTT | |
| | | | | | | (SEQ | | NO: 543) | | | | | | T (SEQ | |
| | | | | | | ID NO: | | | | | | | | ID NO: | |
| | | | | | | 445) | | | | | | | | 744) | |
| 24 | 17 | 2.94 | L-20-R | 20 | 6_3 | aCTaA | atactaga | AGGTA | chr7 | 8656336 | Intron | NXPH1 | 007- | GCATT | 59.148 |
| | | | | | | AAAAa | ggcaaaa | AAGACt | | | | | 008656230 | TAGGA | |
| | | | | | | AgTgT | aaggt | GgAtCA | | | | | | CAGAG | |
| | | | | | | GgA (SEQ | (SEQ ID | T (SEQ | | | | | | AGCGA | |
| | | | | | | ID NO: | NO: | ID NO: | | | | | | AGC | |
| | | | | | | 446) | 594) | 544) | | | | | | (SEQ ID | |
| | | | | | | | | | | | | | | NO: | |
| | | | | | | | | | | | | | | 745) | |
| 25 | 13 | 2.946 | R-20-L | 20 | 5_4 | ATtCT | cttagctc | TGCAGt | chr1 | 187871060 | Intergenic | PLA2G4A | 001- | GGCCC | 61.508 |
| | | | | | | ACTGg | ctgtggtg | aTtTgTT | | | | | 187870884 | TTGCT | |
| | | | | | | CaTTA | ggta | TCAGt | | | | | | ATGCC | |
| | | | | | | CaT (SEQ | (SEQ ID | (SEQ ID | | | | | | AGGGA | |
| | | | | | | ID NO: | NO: | NO: 545) | | | | | | A (SEQ | |
| | | | | | | 447) | 595) | | | | | | | ID NO: | |
| | | | | | | | | | | | | | | 746) | |
| 26 | 5 | 2.947 | R-12-L | 12 | 6_5 | tTGCa | tgttccatc | cGgAG | chr12 | 90462283 | Intergenic | LOC338758 | 012- | CACCA | 61.508 |
| | | | | | | ACTaT | att (SEQ | AtTtTTT | | | | | 090462066 | TTCTC | |
| | | | | | | CcTTtC | ID NO: | TTtAGA | | | | | | CCTGC | |
| | | | | | | Cc (SEQ | 596) | (SEQ ID | | | | | | CTCAG | |
| | | | | | | ID NO: | | NO: 546) | | | | | | C (SEQ | |
| | | | | | | 448) | | | | | | | | ID NO: | |
| | | | | | | | | | | | | | | 747) | |
| 27 | 2 | 2.947 | L-19-R | 19 | 6_6 | TCTcA | aataaaa | gGGTg | chr1 | 117309480 | Intron | CD2 | 001- | CAGGA | 61.508 |
| | | | | | | AAAAa | aacaaaa | AAcAgA | | | | | 117309377 | GGCTG | |
| | | | | | | ACaaa | atagt | GTAGtA | | | | | | AGGTG | |
| | | | | | | aCA (SEQ | (SEQ ID | a (SEQ | | | | | | GGTGA | |
| | | | | | | ID NO: | NO: | ID NO: | | | | | | A (SEQ | |
| | | | | | | 431) | 597) | 547) | | | | | | ID NO: | |
| | | | | | | | | | | | | | | 748) | |
| 28 | 10 | 2.949 | L-11-R | 11 | 6_4 | aaTtAA | tgtccttca | AGGTg | chr3 | 121399914 | Intron | GOLGB1 | 003- | GCTAG | 59.378 |
| | | | | | | AAAcA | ac (SEQ | AAtACA | | | | | 121399809 | CATTT | |
| | | | | | | aTaTG | ID NO: | GTgGtA | | | | | | GCACT | |
| | | | | | | CA (SEQ | 598) | T (SEQ | | | | | | CCTGG | |
| | | | | | | ID NO: | | ID NO: | | | | | | G (SEQ | |
| | | | | | | 449) | | 548) | | | | | | ID NO: | |
| | | | | | | | | | | | | | | 749) | |
| 29 | 8 | 2.95 | R-11-L | 11 | 5_5 | ATcCa | ccgaatttt | TGttGtG | chr1 | 40370608 | Intergenic | MYCL1 | 001- | CACCT | 61.508 |
| | | | | | | cCTGT | tt (SEQ | TtTTTT | | | | | 040370321 | GTCAC | |
| | | | | | | CTTgg | ID NO: | TgAGA | | | | | | CACGC | |
| | | | | | | CCT | 599) | (SEQ ID | | | | | | CTAGC | |
| | | | | | | (SEQ | | NO: | | | | | | T (SEQ | |
| | | | | | | ID NO: | | 549) | | | | | | ID NO: | |
| | | | | | | 450) | | | | | | | | 750) | |
| 30 | 13 | 2.951 | L-15-R | 15 | 5 4 | TCTaA | taattgttta | AGaaAA | chr13 | 19256886 | Intergenic | ANKRD20A9P | 013- | GTACA | 59.578 |
| | | | | | | AAAAG | aaaaa | AGACA | | | | | 019256623 | GAGTA | |
| | | | | | | AaTCa | (SEQ ID | aTAGtA | | | | | | ACCTA | |
| | | | | | | aaA (SEQ | NO: 600) | T (SEQ | | | | | | TAAGT | |
| | | | | | | ID NO: | | ID NO: | | | | | | GTGAC | |
| | | | | | | 451) | | 550) | | | | | | ATACTT | |
| | | | | | | | | | | | | | | TTGC | |
| | | | | | | | | | | | | | | (SEQ ID | |
| | | | | | | | | | | | | | | NO: 751) | |
| 31 | 5 | 2.951 | R-18-L | 18 | 6_5 | AaatTA | ttaaatttgt | TttAGA | chr8 | 104573187 | Intron | RIMS2 | 008- | GGGGA | 59.958 |
| | | | | | | CTaTC | aagcattt | GTtTgT | | | | | 104572889 | TTCAG | |
| | | | | | | TgTAC | (SEQ ID | TTgAtA | | | | | | AGATT | |
| | | | | | | CT | NO: 601) | (SEQ ID | | | | | | GTTAA | |
| | | | | | | (SEQ | | NO: 551) | | | | | | AGAAC | |
| | | | | | | ID NO: | | | | | | | | ACAAG | |
| | | | | | | 452) | | | | | | | | G (SEQ | |
| | | | | | | | | | | | | | | ID NO: | |
| | | | | | | | | | | | | | | 752) | |
| 32 | 20 | 2.955 | R-15-L | 15 | 5_3 | ATGCT | atgtatata | TtCAtAt | chr4 | 116455083 | Intergenic | NDST4 | 004- | GCACC | 59.958 |
| | | | | | | tCTcTa | tacaca | aCTTTT | | | | | 116454862 | TTGTC | |
| | | | | | | TTTAC | (SEQ ID | TCAGt | | | | | | TATTTC | |
| | | | | | | CT (SEQ | NO: 602) | (SEQ ID | | | | | | TTAGA | |
| | | | | | | ID NO: | | NO: 552) | | | | | | GTTGT | |
| | | | | | | 453) | | | | | | | | TGAGC | |
| | | | | | | | | | | | | | | (SEQ ID | |
| | | | | | | | | | | | | | | NO: | |
| | | | | | | | | | | | | | | 753) | |
| 33 | 10 | 2.956 | R-18-L | 18 | 6_4 | ATcCT | agccagc | TGCAGt | chr7 | 98432906 | Intergenic | TMEM130 | 007- | AGTTT | 61.508 |
| | | | | | | AtTtTC | tctttgata | GTgTTT | | | | | 098432808 | GCGTC | |
| | | | | | | TTTAat | ac (SEQ | TTtAGg | | | | | | TGAGC | |
| | | | | | | g (SEQ | ID NO: | (SEQ ID | | | | | | CTGGC | |
| | | | | | | ID NO: | 603) | NO: 553) | | | | | | C (SEQ | |
| | | | | | | 454) | | | | | | | | ID NO: | |
| | | | | | | | | | | | | | | 754) | |
| 34 | 5 | 2.96 | R-18-L | 18 | 6_5 | cTaCa | ttccctga | TGCAtA | chr12 | 107506088 | Intergenic | CRY1 | 012- | CTGAA | 59.378 |
| | | | | | | AaTGa | aagataa | GTtTTg | | | | | 107505826 | TGGTG | |
| | | | | | | CTTTc | caa | TatAGA | | | | | | AGGGC | |
| | | | | | | CCT | (SEQ ID | (SEQ ID | | | | | | TCCCT | |
| | | | | | | (SEQ | NO: | NO: 554) | | | | | | T (SEQ | |
| | | | | | | ID NO: | 604) | | | | | | | ID NO: | |
| | | | | | | 455) | | | | | | | | 755) | |
| 35 | 2 | 2.96 | L-15-R | 15 | 6_6 | gCacA | ggggaat | AGGTtA | chr20 | 4296665 | Intergenic | ADRA1D | 020- | GTCTC | 59.148 |
| | | | | | | AAAAa | acttggta | AaAgAG | | | | | 004296488 | CCAGG | |
| | | | | | | ACTCT | (SEQ ID | TtGaAc | | | | | | GGAAG | |
| | | | | | | aCt (SEQ | NO: 605) | (SEQ ID | | | | | | AATCA | |
| | | | | | | ID NO: | | NO: 555) | | | | | | TAG | |
| | | | | | | 456) | | | | | | | | (SEQ ID | |
| | | | | | | | | | | | | | | NO: | |
| | | | | | | | | | | | | | | 756) | |
| 36 | 5 | 2.965 | R-10-L | 10 | 6_5 | tTGCct | tatttctata | TGtAaA | chr2 | 30843806 | Intron | LCLAT1 | 002- | CAGGT | 61.508 |
| | | | | | | CTGTC | (SEQ ID | GgCTTT | | | | | 030843698 | CCACA | |
| | | | | | | TTcAC | NO: 606) | TTttGg | | | | | | AACCC | |
| | | | | | | Ca | | (SEQ ID | | | | | | CTCCC | |
| | | | | | | (SEQ | | NO: 556) | | | | | | A (SEQ | |
| | | | | | | ID NO: | | | | | | | | ID NO: | |
| | | | | | | 457) | | | | | | | | 757) | |
| 37 | 5 | 2.965 | L-13-R | 13 | 6_5 | aCTcA | gtcacac | AaGTtAt | chr8 | 116589056 | Intron | TRPS1 | 008- | TCACC | 61.508 |
| | | | | | | AAAAc | agcata | TAttAg | | | | | 116588765 | CAGGG | |
| | | | | | | ACTCa | (SEQ ID | GACAG | | | | | | TCACA | |
| | | | | | | GtA | NO: 607) | (SEQ ID | | | | | | CGGCT | |
| | | | | | | (SEQ | | NO: | | | | | | A (SEQ | |
| | | | | | | ID NO: | | 557) | | | | | | ID NO: | |
| | | | | | | 458) | | | | | | | | 758) | |
| 38 | 5 | 2.969 | R-19-L | 19 | 6_5 | cTaaTA | tggagatt | atatGAa | chr4 | 75865784 | Intron | PARM1 | 004- | GGAGG | 61.508 |
| | | | | | | CcaTC | aggatttc | TtTTTT | | | | | 075865654 | GGGCT | |
| | | | | | | TTTAC | aac (SEQ ID | TCAGA | | | | | | GTCTT | |
| | | | | | | CT | NO: 608) | (SEQ ID | | | | | | TCTGG | |
| | | | | | | (SEQ | | NO: 558) | | | | | | T (SEQ | |
| | | | | | | ID NO: | | | | | | | | ID NO: | |
| | | | | | | 459) | | | | | | | | 759) | |
| 39 | 5 | 2.97 | R-17-L | 17 | 6_5 | cTGCa | cctatctg | TGtAGg | chr8 | 110132747 | Intergenic | TRHR | 008- | CTCAG | 60.178 |
| | | | | | | AtTGT | cttgttgtt | tTCTTT | | | | | 110132588 | CCATG | |
| | | | | | | CTTTc | (SEQ ID | TTttGt | | | | | | TCAAG | |
| | | | | | | CtT | NO: | (SEQ ID | | | | | | CAAAT | |
| | | | | | | (SEQ | 609) | NO: | | | | | | CATTC | |
| | | | | | | ID NO: | | 559) | | | | | | AAGC | |
| | | | | | | 460) | | | | | | | | (SEQ ID | |
| | | | | | | | | | | | | | | NO: | |
| | | | | | | | | | | | | | | 760) | |
| 40 | 5 | 2.971 | L-13-R | 13 | 6 5 | TCTtA | gtttcaag | AGGTA | chr13 | 108402040 | Intron | FAM155A | 013- | ATGTT | 59.508 |
| | | | | | | AtcAG | gtcta | AAtAtA | | | | | 108401776 | CCAGG | |
| | | | | | | ACaCT | (SEQ ID | GTcGtA | | | | | | CGCCA | |
| | | | | | | aaA | NO: 610) | g (SEQ | | | | | | CGTAG | |
| | | | | | | (SEQ | | ID NO: | | | | | | (SEQ ID | |
| | | | | | | ID NO: | | 560) | | | | | | NO: | |
| | | | | | | 461) | | | | | | | | 761) | |
| 41 | 10 | 2.972 | R-18-L | 18 | 6_4 | ATGtT | acagtctc | TGgAG | chr5 | 129699260 | Intergenic | CHSY3 | 005- | CAACA | 60.178 |
| | | | | | | AtTaTC | ctcaaga | AtTtTTT | | | | | 129699152 | CAATT | |
| | | | | | | TTTAC | caa | TTCtat | | | | | | CAGCC | |
| | | | | | | Ca (SEQ | (SEQ ID | (SEQ ID | | | | | | ATGTT | |
| | | | | | | ID NO: | NO: | NO: | | | | | | CTTTG | |
| | | | | | | 462) | 611) | 561) | | | | | | TCCC | |
| | | | | | | | | | | | | | | (SEQ ID | |
| | | | | | | | | | | | | | | NO: | |
| | | | | | | | | | | | | | | 762) | |
| 42 | 5 | 2.973 | R-16-L | 16 | 6_5 | tTGacA | ctttctcct | TGCAGt | chr4 | 61094915 | Intergenic | LPHN3 | 004- | TAGGG | 61.508 |
| | | | | | | CccTC | agtagtc | GTCTaT | | | | | 061094798 | GGCAC | |
| | | | | | | TTTcC | (SEQ ID | TTttGc | | | | | | CTGTG | |
| | | | | | | CT (SEQ | NO: 612) | (SEQ ID | | | | | | CCAGT | |
| | | | | | | ID NO: | | NO: 562) | | | | | | T (SEQ | |
| | | | | | | 463) | | | | | | | | ID NO: | |
| | | | | | | | | | | | | | | 763) | |
| 43 | 17 | 2.974 | L-14-R | 14 | 6_3 | TCTcA | ctagctat | AaGTgc | chr10 | 59017381 | Intergenic | MIR3924 | 010- | GGCCA | 61.508 |
| | | | | | | AAAAG | caactc | tGtCAG | | | | | 059017278 | TCCCT | |
| | | | | | | ACTCa | (SEQ ID | TAGtAT | | | | | | CTACT | |
| | | | | | | GCc (SEQ | NO: 613) | (SEQ ID | | | | | | CCTGG | |
| | | | | | | ID NO: | | NO: 563) | | | | | | T (SEQ | |
| | | | | | | 464) | | | | | | | | ID NO: | |
| | | | | | | | | | | | | | | 764) | |
| 44 | 5 | 2.975 | L-18-R | 18 | 6_5 | aaTaA | aggtaag | AGaaAA | chr2 | 162041661 | Intron | TANK | 002- | GAAGA | 60.908 |
| | | | | | | AAActA | aaacaaa | AGAatG | | | | | 162041530 | TCAGT | |
| | | | | | | CTCTa | ttat | TAGtAT | | | | | | CACTT | |
| | | | | | | CA (SEQ | (SEQ ID | (SEQ ID | | | | | | GGCCT | |
| | | | | | | ID NO: | NO: | NO: 564) | | | | | | CTGG | |
| | | | | | | 465) | 614) | | | | | | | (SEQ ID | |
| | | | | | | | | | | | | | | NO: 765) | |
| 45 | 13 | 2.976 | L-10-R | 10 | 5_4 | TCTcA | tttatttcct | AGGTA | chr4 | 76823285 | Intron | PPEF2 | 004- | GGGCA | 61.508 |
| | | | | | | AAAca | (SEQ ID | AAaAC | | | | | 076823183 | GAATG | |
| | | | | | | ACTCT | NO: | AtaAGtg | | | | | | GTCAC | |
| | | | | | | GtA | 615) | T | | | | | | CCAGC | |
| | | | | | | (SEQ | | (SEQ | | | | | | A (SEQ | |
| | | | | | | ID NO: | | ID NO: | | | | | | ID NO: | |
| | | | | | | 466) | | 565) | | | | | | 766) | |
| 46 | 26 | 2.976 | R-19-L | 19 | 6_2 | ATGaa | tcttgataa | TGCAG | chr13 | 76308711 | Intron | LMO7 | 013- | GTGCA | 60.438 |
| | | | | | | AaTGT | gggtgttg | AGTgTT | | | | | 076308608 | TTCAG | |
| | | | | | | CaTTA | gt (SEQ | TcTCAG | | | | | | GAAGC | |
| | | | | | | aaT | ID NO: | A (SEQ | | | | | | TTCTG | |
| | | | | | | (SEQ | 616) | ID NO: | | | | | | TTTGA | |
| | | | | | | ID NO: | | 566) | | | | | | CC | |
| | | | | | | 467) | | | | | | | | (SEQ ID | |
| | | | | | | | | | | | | | | NO: | |
| | | | | | | | | | | | | | | 767) | |
| 47 | 8 | 2.977 | R-11-L | 11 | 5_5 | tTcCTt | tcacccct | TcaAGA | chr8 | 106290875 | Intergenic | ZFPM2 | 008- | GCAGG | 59.378 |
| | | | | | | CTcTC | aca | tTtTgTT | | | | | 106290748 | AAGTG | |
| | | | | | | TTTcC | (SEQ ID | TCAGA | | | | | | CCAGG | |
| | | | | | | CT (SEQ | NO: 617) | (SEQ ID | | | | | | ACATT | |
| | | | | | | ID NO: | | NO: 567) | | | | | | G (SEQ | |
| | | | | | | 468) | | | | | | | | ID NO: | |
| | | | | | | | | | | | | | | 768) | |
| 48 | 8 | 2.977 | R-16-L | 16 | 5_5 | tTaCTA | cctctggt | TGCAG | chr9 | 113113186 | Intergenic | TXNDC8 | 009- | GCTGG | 61.508 |
| | | | | | | aTGcC | cacaaca | AGaCT | | | | | 113113045 | AGGTG | |
| | | | | | | TTTtC | t (SEQ | TgTgC | | | | | | ATGGC | |
| | | | | | | CT | ID NO: | Gt (SEQ | | | | | | CAAGG | |
| | | | | | | (SEQ | 618) | ID NO: | | | | | | T (SEQ | |
| | | | | | | ID NO: | | 568) | | | | | | ID NO: | |
| | | | | | | 469) | | | | | | | | 769) | |
| 49 | 17 | 2.979 | R-20-L | 20 | 6_3 | ATcCT | ctcaaatc | TGCAG | chr13 | 103006085 | Intron | FGF14 | 013- | CCCAG | 61.508 |
| | | | | | | ACacT | tattctcca | AGTgTT | | | | | 103005959 | TACTC | |
| | | | | | | CTcgA | ccc (SEQ ID | TTTCct | | | | | | CCACA | |
| | | | | | | CCc | NO: | A (SEQ | | | | | | CTCTG | |
| | | | | | | (SEQ | 619) | ID NO: | | | | | | C (SEQ | |
| | | | | | | ID NO: | | 569) | | | | | | ID NO: | |
| | | | | | | 470) | | | | | | | | 770) | |
| 50 | 8 | 2.98 | L-11-R | 11 | 5_5 | ctTGcA | ttctgaac | AGGTg | chr9 | 83220345 | Intergenic | TLE4 | 009- | GTCCA | 61.088 |
| | | | | | | tAAaA | aaa | AAGAC | | | | | 083220145 | GGACA | |
| | | | | | | CTCTG | (SEQ ID | AGTttt | | | | | | AAGCT | |
| | | | | | | CA | NO: 620) | tT (SEQ | | | | | | CAGTA | |
| | | | | | | (SEQ | | ID NO: | | | | | | G | |
| | | | | | | ID NO: | | 570) | | | | | | CC | |
| | | | | | | 471) | | | | | | | | (SEQ ID | |
| | | | | | | | | | | | | | | NO: | |
| | | | | | | | | | | | | | | 771) | |

SIRT6-DdCBE

| | | | | | | | | | | | | | | | |
|---|---|---|---|---|---|---|---|---|---|---|---|---|---|---|---|
| 1 | 10 | 2.494 | R-11-L | 11 | 6_4 | aTTCA | tggcattct | GAgAG | chr1 | 112447620 | Intron | KCND3 | 001- | CAGCT | 61.508 |
| | | | | | | CGCC | ca (SEQ | ctCttCC | | | | | 112447519 | GGCCA | |
| | | | | | | GGAa | ID NO: | CCGTAc | | | | | | AGCAG | |
| | | | | | | GtCCa | 621) | (SEQ ID | | | | | | GGATC | |
| | | | | | | (SEQ | | NO: 672) | | | | | | A (SEQ | |
| | | | | | | ID NO: | | | | | | | | ID NO: | |
| | | | | | | 472) | | | | | | | | 772) | |
| 2 | 2 | 2.513 | L-19-R | 19 | 6_6 | acACa | gcatttctg | AGGatC | chr5 | 173100953 | Intergenic | BOD1 | 005- | GGGGG | 61.508 |
| | | | | | | gGGC | ccgtttag | TCtaGt | | | | | 173100857 | AGTCG | |
| | | | | | | GaaGC | aa (SEQ | GTGAA | | | | | | GACTT | |
| | | | | | | TGTC | ID NO: | g (SEQ | | | | | | AGAAG | |
| | | | | | | (SEQ | 622) | ID NO: | | | | | | G (SEQ | |
| | | | | | | ID NO: | | 673) | | | | | | ID NO: | |
| | | | | | | 473) | | | | | | | | 773) | |
| 3 | 13 | 2.546 | R-16-L | 16 | 5_4 | aTaCA | actggaa | GAaAa | chr10 | 6751406 | Intergenic | LOC100507127 | 010- | TGCTA | 59.508 |
| | | | | | | aGCC | gcaagag | CCCaG | | | | | 006751175 | GGCCC | |
| | | | | | | GGAG | gg (SEQ | CCGgG | | | | | | CGATT | |
| | | | | | | aGCCT | ID NO: | TAt | | | | | | TGTGG | |
| | | | | | | (SEQ | 623) | (SEQ ID | | | | | | (SEQ ID | |
| | | | | | | ID NO: | | NO: | | | | | | NO: | |
| | | | | | | 474) | | 674) | | | | | | 774) | |
| 4 | 2 | 2.62 | L-17-R | 17 | 6_6 | gTACa | gaggcgc | AGGCC | chrX | 69669589 | Exon | DLG3 | 00X- | GACCC | 60.908 |
| | | | | | | CaGCc | tgaagga | CTgtGG | | | | | 069669412 | TTCCA | |
| | | | | | | GGGC | ggc | tGcGAtt | | | | | | TTAGA | |
| | | | | | | gGTg | (SEQ ID | (SEQ ID | | | | | | TCTGA | |
| | | | | | | (SEQ | NO: 624) | NO: | | | | | | GGGC | |
| | | | | | | ID NO: | | 675) | | | | | | (SEQ ID | |
| | | | | | | 475) | | | | | | | | NO: 775) | |
| 5 | 10 | 2.627 | R-14-L | 14 | 6_4 | aTTCA | cgtgtgca | GgCAG | chr20 | 61295340 | Intron | LOC100127888 | 020- | GTGTA | 61.508 |
| | | | | | | CcCCC | gggaca | CCCCG | | | | | 061295138 | CCCCA | |
| | | | | | | cAGGG | (SEQ ID | aCGCac | | | | | | GAGAG | |
| | | | | | | CCT | NO: 625) | tg (SEQ | | | | | | AGCCG | |
| | | | | | | (SEQ | | ID NO: | | | | | | A (SEQ | |
| | | | | | | ID NO: | | 676) | | | | | | ID NO: | |
| | | | | | | 476) | | | | | | | | 776) | |
| 6 | 5 | 2.644 | L-19-R | 19 | 6_5 | TacCtC | atagtccc | AGGCt | chr7 | 142566992 | Intron | EPHB6 | 007- | CCTCA | 61.288 |
| | | | | | | aGCcG | tgaaagg | CTCCtG | | | | | 142566895 | GGTGA | |
| | | | | | | GGgTG | aggg | tGTGAt | | | | | | GAGCA | |
| | | | | | | TC (SEQ | (SEQ ID | g (SEQ | | | | | | CAGCC | |
| | | | | | | ID NO: | NO: 626) | ID NO: | | | | | | TT | |
| | | | | | | 477) | | 677) | | | | | | (SEQ ID | |
| | | | | | | | | | | | | | | NO: | |
| | | | | | | | | | | | | | | 777) | |
| 7 | 2 | 2.649 | L-10-R | 10 | 6_6 | cTcCc | cgtgtggc | AaGCCt | chr19 | 8456702 | Intron | RAB11B | 019- | GGATC | 61.508 |
| | | | | | | CacCt | ga (SEQ | TCCGG | | | | | 008456519 | CAAGG | |
| | | | | | | GGGC | ID NO: | gGgGAg | | | | | | TGCCT | |
| | | | | | | TGTC | 627) | g (SEQ | | | | | | TCACC | |
| | | | | | | (SEQ | | ID NO: | | | | | | C (SEQ | |
| | | | | | | ID NO: | | 678) | | | | | | ID NO: | |
| | | | | | | 478) | | | | | | | | 778) | |
| 8 | 17 | 2.65 | L-11-R | 11 | 6_3 | aTgCG | agtgaga | AaGCC | chr6 | 911829 | Intergenic | LOC285768 | 006- | GCTGG | 60.908 |
| | | | | | | CaGaG | aagt | CTCCa | | | | | 000911603 | ATTCG | |
| | | | | | | GaGCT | (SEQ ID | GCGTG | | | | | | ATCTG | |
| | | | | | | GTg | NO: 628) | AAg | | | | | | AGGTC | |
| | | | | | | (SEQ | | (SEQ ID | | | | | | AGCT | |
| | | | | | | ID NO: | | NO: 679) | | | | | | (SEQ ID | |
| | | | | | | 479) | | | | | | | | NO: 779) | |
| 9 | 2 | 2.662 | R-18-L | 18 | 6_6 | tTTCA | aatctgtta | GACAG | chr10 | 88121549 | Intron | GRID1 | 010- | CCCAG | 62.768 |
| | | | | | | CGCCc | aagctgtg | CttCtCC | | | | | 088121363 | GACAA | |
| | | | | | | aAtGG | t (SEQ | tttTAA | | | | | | AGTTG | |
| | | | | | | Caa | ID NO: | (SEQ ID | | | | | | CTTTG | |
| | | | | | | (SEQ | 629) | NO: | | | | | | GGGC | |
| | | | | | | ID NO: | | 680) | | | | | | (SEQ ID | |
| | | | | | | 480) | | | | | | | | NO: | |
| | | | | | | | | | | | | | | 780) | |
| 10 | 2 | 2.671 | L-17-R | 17 | 6_6 | TTCCG | ttgttgtgg | AGGtgt | chr5 | 2112252 | Intergenic | IRX4 | 005- | GCCTC | 61.508 |
| | | | | | | CcGCG | gagaaca | TCtGGC | | | | | 002112000 | GCCAA | |
| | | | | | | GaGaT | a (SEQ | tTGAgC | | | | | | GAAAC | |
| | | | | | | taC | ID NO: | (SEQ ID | | | | | | GCCCA | |
| | | | | | | (SEQ | 630) | NO: 681) | | | | | | T (SEQ | |
| | | | | | | ID NO: | | | | | | | | ID NO: | |
| | | | | | | 481) | | | | | | | | 781) | |
| 11 | 8 | 2.673 | L-18-R | 18 | 5 5 | aaACG | gatgacgt | AGGCC | chr2 | 2737282 | Intron | TCF23 | 002- | CCAGC | 61.088 |
| | | | | | | CGGC | aggtggg | CTCtGG | | | | | 027372656 | ACACC | |
| | | | | | | aGGG | cag | tGcctAC | | | | | | CTGAT | |
| | | | | | | CTGca | (SEQ ID | (SEQ ID | | | | | | AAGCA | |
| | | | | | | (SEQ | NO: | NO: 682) | | | | | | GGA | |
| | | | | | | ID NO: | 631) | | | | | | | (SEQ ID | |
| | | | | | | 482) | | | | | | | | NO: 782) | |
| 12 | 2 | 2.675 | L-15-R | 15 | 6_6 | TTcCtC | cagggca | AGGgg | chr2 | 27421788 | Intergenic | SLC5A6 | 002- | CCAGG | 60.908 |
| | | | | | | caCaG | gaggtga | CTggG | | | | | 027421674 | GCAAC | |
| | | | | | | GGCT | a (SEQ | GtGTGA | | | | | | AGGTT | |
| | | | | | | GgC (SEQ | ID NO: | cC | | | | | | TAAGA | |
| | | | | | | ID NO: | 632) | (SEQ ID | | | | | | CCCA | |
| | | | | | | 483) | | NO: 683) | | | | | | (SEQ ID | |
| | | | | | | | | | | | | | | NO: 783) | |
| 13 | 5 | 2.68 | R-10-L | 10 | 6 5 | GcTCA | cctttgcct | GACAG | chr17 | 18188907 | Intron | TOP3A | 017- | GGTCT | 61.508 |
| | | | | | | CcCCGg | g (SEQ | CCtatgC | | | | | 018188800 | CGATG | |
| | | | | | | GAatcC | ID NO: | agGTAA | | | | | | TGCTC | |
| | | | | | | CT (SEQ | 633) | (SEQ ID | | | | | | CGCAT | |
| | | | | | | ID NO: | | NO: 684) | | | | | | G (SEQ | |
| | | | | | | 484) | | | | | | | | ID NO: | |
| | | | | | | | | | | | | | | 784) | |
| 14 | 5 | 2.68 | R-11-L | 11 | 6_5 | GTTCt | cattctctt | GACAG | chr16 | 13033690 | Intron | SHISA9 | 016- | CCATC | 61.508 |
| | | | | | | CtCCG | ga (SEQ | CaatGCt | | | | | 013033556 | ACCTG | |
| | | | | | | cAGaa | ID NO: | GaGaA | | | | | | CCTCT | |
| | | | | | | CCT (SEQ | 634) | A (SEQ | | | | | | CCCTG | |
| | | | | | | ID NO: | | ID NO: | | | | | | T (SEQ | |
| | | | | | | 485) | | 685) | | | | | | ID NO: | |
| | | | | | | | | | | | | | | 785) | |
| 15 | 5 | 2.684 | L-19-R | 19 | 6 5 | TTAtGa | aggaact | AGGCC | chr5 | 52333431 | Intron | ITGA2 | 005- | CTTGG | 61.088 |
| | | | | | | GGgtG | aggatttc | tTagGGt | | | | | 052333300 | TGTGT | |
| | | | | | | aGtTG | accg | GTGAAt | | | | | | CTCTT | |
| | | | | | | TC (SEQ | (SEQ ID | (SEQ ID | | | | | | CCTGC | |
| | | | | | | ID NO: | NO: | NO: 686) | | | | | | ACG | |
| | | | | | | 486) | 635) | | | | | | | (SEQ ID | |
| | | | | | | | | | | | | | | NO: 786) | |
| 16 | 2 | 2.688 | L-11-R | 11 | 6_6 | TcACc | atgcactt | AGGgag | chr20 | 20629814 | Intron | RALGAPA2 | 020- | GGTAA | 61.288 |
| | | | | | | CaGCc | ctg | TCCtGt | | | | | 020629697 | AAGCA | |
| | | | | | | tGGCT | (SEQ ID | GTGAA | | | | | | CCCTG | |
| | | | | | | GTg | NO: 636) | g (SEQ | | | | | | AGAGG | |
| | | | | | | (SEQ | | ID NO: | | | | | | GC | |
| | | | | | | ID NO: | | 687) | | | | | | (SEQ ID | |
| | | | | | | 487) | | | | | | | | NO: | |
| | | | | | | | | | | | | | | 787) | |
| 17 | 5 | 2.689 | L-13-R | 13 | 6_5 | cTcaG | tttacatag | tGGCtC | chr22 | 50573100 | Intron | MOV10L1 | 022- | CAGTC | 59.508 |
| | | | | | | CGaC | gggg | agCGG | | | | | 050572985 | TCCAC | |
| | | | | | | GtGGC | (SEQ ID | CGTGA | | | | | | AAGTG | |
| | | | | | | TGTC | NO: 637) | ct (SEQ | | | | | | ACGGG | |
| | | | | | | (SEQ | | ID NO: | | | | | | (SEQ ID | |
| | | | | | | ID NO: | | 688) | | | | | | NO: | |
| | | | | | | 488) | | | | | | | | 788) | |
| 18 | 5 | 2.691 | R-13-L | 13 | 6_5 | acTCc | cgttccca | GcCAG | chr20 | 2777030 | Exon | CPXM1 | 020- | AGGGC | 61.508 |
| | | | | | | CGCCc | tgcat | CCaCGt | | | | | 002776931 | AGCAG | |
| | | | | | | cAGGG | (SEQ ID | aGCGc | | | | | | GTGAA | |
| | | | | | | CCT | NO: 638) | Ac (SEQ | | | | | | TGCGC | |
| | | | | | | (SEQ | | ID NO: | | | | | | A (SEQ | |
| | | | | | | ID NO: | | 689) | | | | | | ID NO: | |
| | | | | | | 489) | | | | | | | | 789) | |
| 19 | 5 | 2.692 | L-19-R | 19 | 6_5 | gaAgG | acctgtcc | AGGCt | chr14 | 40674202 | Intergenic | FBXO33 | 014- | GGCCC | 61.508 |
| | | | | | | gGGC | cagtctcc | CTgtGG | | | | | 040674014 | TGGCT | |
| | | | | | | GGGG | tgc (SEQ ID | CaTGA | | | | | | TGGCC | |
| | | | | | | CTtcC | NO: 639) | Aa | | | | | | TTAACT | |
| | | | | | | (SEQ | | (SEQ ID | | | | | | (SEQ ID | |
| | | | | | | ID NO: | | NO: | | | | | | NO: | |
| | | | | | | 490) | | 690) | | | | | | 790) | |
| 20 | 5 | 2.693 | R-16-L | 16 | 6_5 | CTTCc | ctccgtgtt | GACAGt | chr17 | 4273323 | Intergenic | UBE2G1 | 017- | GCCAC | 61.508 |
| | | | | | | CaCCa | cccgtgg | CtCGC | | | | | 004273151 | TCCCC | |
| | | | | | | GctGG | (SEQ ID | CaCccA | | | | | | TTTGG | |
| | | | | | | CCT | NO: 640) | A (SEQ | | | | | | TGCTG | |
| | | | | | | (SEQ | | ID NO: | | | | | | T (SEQ | |
| | | | | | | ID NO: | | 691) | | | | | | ID NO: | |
| | | | | | | 491) | | | | | | | | 791) | |
| 21 | 10 | 2.696 | R-14-L | 14 | 6_4 | GTagg | ggcggca | GACAG | chr6 | 157557363 | Intergenic | ARID1B | 006- | CCAGG | 61.288 |
| | | | | | | CGCC | gggaccc | CtCtGC | | | | | 157557252 | AGAAG | |
| | | | | | | GGcG | (SEQ ID | CGCGg | | | | | | AAGTG | |
| | | | | | | GagCT | NO: | Ac (SEQ | | | | | | GCATC | |
| | | | | | | (SEQ | 641) | ID NO: | | | | | | CG | |
| | | | | | | ID NO: | | 692) | | | | | | (SEQ ID | |
| | | | | | | 492) | | | | | | | | NO: 792) | |
| 22 | 2 | 2.7 | R-14-L | 14 | 6_6 | tTTCA | gtttagag | aACAG | chr6 | 12344255 | Intergenic | EDN1 | 006- | CCTTT | 61.508 |
| | | | | | | CaCaG | caaata | CCCatt | | | | | 012344151 | GCAAT | |
| | | | | | | GctGtC | (SEQ ID | CGtGTA | | | | | | GGAGG | |
| | | | | | | CT | NO: 642) | t (SEQ | | | | | | GCAGG | |
| | | | | | | (SEQ | | ID NO: | | | | | | G (SEQ | |
| | | | | | | ID NO: | | 693) | | | | | | ID NO: | |
| | | | | | | 493) | | | | | | | | 793) | |
| 23 | 5 | 2.701 | R-10-L | 10 | 6_5 | GgTCA | ctgaagg | CACAG | chr5 | 28950449 | Intergenic | LSP1P3 | 005- | CAGGC | 59.148 |
| | | | | | | CGCa | agc | CtCtGC | | | | | 028950211 | CTAAG | |
| | | | | | | GaAG | (SEQ ID | aGtGTA | | | | | | CCTTG | |
| | | | | | | GGtCc | NO: | t (SEQ | | | | | | ATAGA | |
| | | | | | | (SEQ | 643) | ID NO: | | | | | | CAG | |
| | | | | | | ID NO: | | 694) | | | | | | (SEQ ID | |
| | | | | | | 494) | | | | | | | | NO: 794) | |
| 24 | 2 | 2.704 | R-14-L | 14 | 6_6 | cTaCA | ccgcagc | GAgAa | chr15 | 91565756 | Exon | VPS33B | 015- | CAAGA | 60.908 |
| | | | | | | CcCCG | gtacgag | CCCCG | | | | | 091565655 | GAGCT | |
| | | | | | | CAGaG | (SEQ ID | CCttcTA | | | | | | ACTAC | |
| | | | | | | aCT | NO: 644) | c (SEQ | | | | | | CTCGG | |
| | | | | | | (SEQ | | ID NO: | | | | | | AGCA | |
| | | | | | | ID NO: | | 695) | | | | | | (SEQ ID | |
| | | | | | | 495) | | | | | | | | NO: 795) | |
| 25 | 2 | 2.712 | R-17-L | 17 | 6_6 | aTTCct | cgctgctg | GAtAGt | chr14 | 65346678 | Intergenic | CHURC1- | 014- | CTCAC | 61.508 |
| | | | | | | GCCG | ccgggac | CCCGC | | | | FNTB | 065346505 | CTGCC | |
| | | | | | | GgGGa | ca (SEQ | gGCGg | | | | | | CTGGG | |
| | | | | | | CaT | ID NO: | gg (SEQ | | | | | | ACTGA | |
| | | | | | | (SEQ | 645) | ID NO: | | | | | | A (SEQ | |
| | | | | | | ID NO: | | 696) | | | | | | ID NO: | |
| | | | | | | 496) | | | | | | | | 796) | |
| 26 | 5 | 2.712 | R-12-L | 12 | 6_5 | cTTCc | gagcctg | GAtAGC | chr6 | 44022923 | Intergenic | C6orf223 | 006- | ACAAC | 61.508 |
| | | | | | | CGCCa | actca | CCaGC | | | | | 044022749 | TCCTG | |
| | | | | | | GAGta | (SEQ ID | CcCagA | | | | | | GGGGG | |
| | | | | | | CCa | NO: 646) | A (SEQ | | | | | | TCCAG | |
| | | | | | | (SEQ | | ID NO: | | | | | | A (SEQ | |
| | | | | | | ID NO: | | 697) | | | | | | ID NO: | |
| | | | | | | 497) | | | | | | | | 797) | |
| 27 | 2 | 2.714 | R-13-L | 13 | 6_6 | tTTCcC | acacctgt | GcCtGC | chr4 | 110690930 | Intron | CFI | 004- | GCTGG | 61.288 |
| | | | | | | tCCtGA | ctgtt | CCtGCt | | | | | 110690821 | CTGCT | |
| | | | | | | GGaC | (SEQ ID | GCcTAc | | | | | | CATGG | |
| | | | | | | Cc | NO: 647) | (SEQ ID | | | | | | TCCAT | |
| | | | | | | (SEQ | | NO: 698) | | | | | | GT | |
| | | | | | | ID NO: | | | | | | | | (SEQ ID | |
| | | | | | | 498) | | | | | | | | NO: | |
| | | | | | | | | | | | | | | 798) | |
| 28 | 2 | 2.715 | R-18-L | 18 | 6_6 | cTcCA | tgcttgcat | GcCAG | chr8 | 41136137 | Intron | SFRP1 | 008- | CCCTG | 61.508 |
| | | | | | | CcCCc | gtgcccg | CCCtG | | | | | 041136008 | CCTGC | |
| | | | | | | agGGG | ag (SEQ | CaGgGc | | | | | | AATGG | |
| | | | | | | CCT | ID NO: | Ag | | | | | | TGAGC | |
| | | | | | | (SEQ | 648) | (SEQ ID | | | | | | T (SEQ | |
| | | | | | | ID NO: | | NO: 699) | | | | | | ID NO: | |
| | | | | | | 499) | | | | | | | | 799) | |
| 29 | 2 | 2.717 | L-16-R | 16 | 6_6 | TTAtaC | gctggcat | tGcCCC | chr4 | 112085926 | Intergenic | PITX2 | 004- | CTGTG | 61.288 |
| | | | | | | aGCaG | ctggcag | TCtGGg | | | | | 112085748 | GGTGC | |
| | | | | | | aGCTG | g (SEQ | aTGAAg | | | | | | AGCTT | |
| | | | | | | Tg | ID NO: | (SEQ ID | | | | | | CAGCA | |
| | | | | | | (SEQ | 649) | NO: | | | | | | GA | |
| | | | | | | ID NO: | | 700) | | | | | | (SEQ ID | |
| | | | | | | 500) | | | | | | | | NO: 800) | |
| 30 | 2 | 2.718 | R-17-L | 17 | 6_6 | GcaCA | aggcaca | GAgAcg | chr2 | 202897610 | Intergenic | FZD7 | 002- | GCGCC | 61.508 |
| | | | | | | CGCC | cggggcc | CCCGg | | | | | 202897403 | TGGCT | |
| | | | | | | GcAGa | act | CGCGT | | | | | | CAAGA | |
| | | | | | | aCCa | (SEQ ID | cg (SEQ | | | | | | CGAAG | |
| | | | | | | (SEQ | NO: 650) | ID NO: | | | | | | A (SEQ | |
| | | | | | | ID NO: | | 701) | | | | | | ID NO: | |
| | | | | | | 501) | | | | | | | | 801) | |
| 31 | 5 | 2.718 | L-10-R | 10 | 6_5 | TcACG | caggtagt | AGGgC | chr17 | 18864117 | Intron | SLC5A10 | 017- | TCTGT | 61.508 |
| | | | | | | CaGC | cc (SEQ | CcCCG | | | | | 018864014 | GGGCC | |
| | | | | | | GaGtC | ID NO: | GgGTGt | | | | | | CCTCA | |
| | | | | | | TGga | 651) | Ag | | | | | | AGAGG | |
| | | | | | | (SEQ | | (SEQ ID | | | | | | A (SEQ | |
| | | | | | | ID NO: | | NO: 702) | | | | | | ID NO: | |
| | | | | | | 502) | | | | | | | | 802) | |
| 32 | 5 | 2.719 | L-16-R | 16 | 6_5 | cTACc | catgtact | AGGaC | chr9 | 111625199 | Exon | ACTL7A | 009- | CGGTC | 61.508 |
| | | | | | | aGtCG | cctatgga | CTCCG | | | | | 111625099 | TTGGT | |
| | | | | | | cGcCT | (SEQ ID | GCcTG | | | | | | TTCAG | |
| | | | | | | GTC | NO: 652) | gtg | | | | | | ACCCG | |
| | | | | | | (SEQ | | (SEQ ID | | | | | | C (SEQ | |
| | | | | | | ID NO: | | NO: | | | | | | ID NO: | |
| | | | | | | 503) | | 703) | | | | | | 803) | |
| 33 | 2 | 2.72 | R-17-L | 17 | 6_6 | caTCA | actccact | tACAGC | chr17 | 34250712 | Intron | RDM1 | 017- | CCGAG | 61.288 |
| | | | | | | CcCCc | ccattagc | CCtGC | | | | | 034250614 | ATCAC | |
| | | | | | | GgGaGc | (SEQ | CtCtgAc | | | | | | GCCAG | |
| | | | | | | CCT | ID NO: | (SEQ ID | | | | | | TGCAC | |
| | | | | | | (SEQ | 653) | NO: | | | | | | TT | |
| | | | | | | ID NO: | | 704) | | | | | | (SEQ ID | |
| | | | | | | 504) | | | | | | | | NO: 804) | |
| 34 | 2 | 2.722 | L-13-R | 13 | 6_6 | aTcCa | ctcttgac | AGaCtg | chr1 | 177992803 | Intron | LOC730102 | 001- | GGGGA | 61.508 |
| | | | | | | CaGCt | cttgc | gCaGG | | | | | 177992627 | AGGGG | |
| | | | | | | GGGC | (SEQ ID | aGTGA | | | | | | AAAGA | |
| | | | | | | aGTC | NO: 654) | AC | | | | | | GGCAG | |
| | | | | | | (SEQ | | (SEQ ID | | | | | | T (SEQ | |
| | | | | | | ID NO: | | NO: | | | | | | ID NO: | |
| | | | | | | 505) | | 705) | | | | | | 805) | |
| 35 | 2 | 2.726 | L-15-R | 15 | 6_6 | gTACc | ccctccttc | AGGga | chr3 | 185419730 | Intron | IGF2BP2 | 003- | GCTGG | 61.508 |
| | | | | | | CaGga | cccggg | CTCtGG | | | | | 185419507 | ACTGT | |
| | | | | | | GGcCT | (SEQ ID | CaTGtc | | | | | | CGCAC | |
| | | | | | | GTC | NO: 655) | C (SEQ | | | | | | CTCTG | |
| | | | | | | (SEQ | | ID NO: | | | | | | A (SEQ | |
| | | | | | | ID NO: | | 706) | | | | | | ID NO: | |
| | | | | | | 506) | | | | | | | | 806) | |
| 36 | 2 | 2.727 | R-15-L | 15 | 6_6 | tgaCAa | cctgtcga | GtCAG | chr19 | 5712298 | Intron | LONP1 | 019- | GTCCC | 61.508 |
| | | | | | | GCCcc | aaacggt | CCtgGC | | | | | 005712198 | CTCCA | |
| | | | | | | AGGG | (SEQ ID | CGgGc | | | | | | CCCCT | |
| | | | | | | CCT | NO: 656) | Ac (SEQ | | | | | | TAGAC | |
| | | | | | | (SEQ | | ID NO: | | | | | | T (SEQ | |
| | | | | | | ID NO: | | 707) | | | | | | ID NO: | |
| | | | | | | 507) | | | | | | | | 807) | |
| 37 | 5 | 2.727 | L-18-R | 18 | 6_5 | TTACa | attagggt | AGGCa | chr2 | 134742851 | Intergenic | MIR3679 | 002- | CCTTC | 59.148 |
| | | | | | | gGGCa | gaggaga | CTCaG | | | | | 134742567 | CTCTG | |
| | | | | | | GGaCT | gca | GCGca | | | | | | TGGTG | |
| | | | | | | agC | (SEQ ID | AAa | | | | | | TGACT | |
| | | | | | | (SEQ | NO: | (SEQ ID | | | | | | TTC | |
| | | | | | | ID NO: | 657) | NO: 708) | | | | | | (SEQ ID | |
| | | | | | | 508) | | | | | | | | NO: 808) | |
| 38 | 2 | 2.729 | L-15-R | 15 | 6_6 | ccAgG | cgtctgatt | gGaCtC | chr4 | 1299150 | Intron | MAEA | 004- | GGAGC | 61.288 |
| | | | | | | CGGC | gctgga | TtCGGt | | | | | 001299047 | AAGTG | |
| | | | | | | GcGG | (SEQ ID | GTGgA | | | | | | GTCCC | |
| | | | | | | CccTC | NO: 658) | C (SEQ | | | | | | TGTGG | |
| | | | | | | (SEQ | | ID NO: | | | | | | AA | |
| | | | | | | ID NO: | | 709) | | | | | | (SEQ ID | |
| | | | | | | 509) | | | | | | | | NO: 809) | |
| 39 | 2 | 2.734 | L-20-R | 20 | 6_6 | TTAgG | gaatcata | AGGgC | chr5 | 23747047 | Intergenic | PRDM9 | 005- | GGGAA | 59.378 |
| | | | | | | gaaCa | gcagaca | CTCtaG | | | | | 023746858 | TGCTG | |
| | | | | | | GGGC | gcaac | tGTGcA | | | | | | GCACT | |
| | | | | | | TGTt | (SEQ ID | a (SEQ | | | | | | GTCTA | |
| | | | | | | (SEQ | NO: 659) | ID NO: | | | | | | G (SEQ | |
| | | | | | | ID NO: | | 710) | | | | | | ID NO: | |
| | | | | | | 510) | | | | | | | | 810) | |
| 40 | 2 | 2.735 | L-16-R | 16 | 6_6 | aTACG | ggtggcc | AGGaC | chr11 | 67399253 | Exon | TBX10 | 011- | GGGCT | 61.508 |
| | | | | | | tGaCa | ggggcca | CTCaG | | | | | 067399064 | TCTGG | |
| | | | | | | GGcCT | gc | GtGgGg | | | | | | CATCA | |
| | | | | | | GTa | (SEQ | gC | | | | | | CTGGG | |
| | | | | | | (SEQ | ID NO: | (SEQ ID | | | | | | A (SEQ | |
| | | | | | | ID NO: | 660) | NO: 711) | | | | | | ID NO: | |
| | | | | | | 511) | | | | | | | | 811) | |
| 41 | 10 | 2.739 | R-10-L | 10 | 6_4 | GTgCt | gaaaatct | GcCAG | chr20 | 44452703 | Exon | TNNC2 | 020- | CGCTC | 61.508 |
| | | | | | | CcCCG | ca | CtCCtC | | | | | 044452600 | TTCCC | |
| | | | | | | GAGGc | (SEQ | CGgGT | | | | | | AAGCT | |
| | | | | | | CCT | ID NO: | cg (SEQ | | | | | | CCCGT | |
| | | | | | | (SEQ | 661) | ID NO: | | | | | | T (SEQ | |
| | | | | | | ID NO: | | 712) | | | | | | ID NO: | |
| | | | | | | 512) | | | | | | | | 812) | |
| 42 | 10 | 2.743 | L-14-R | 14 | 6_4 | TgACc | gcacctg | AGGCC | chr17 | 8013212 | Exon | ALOXE3 | 017- | GGTGT | 63.028 |
| | | | | | | CGGC | gtccacg | CTCgG | | | | | 008013113 | TCTCA | |
| | | | | | | aGGGa | (SEQ ID | GgtTGA | | | | | | CACCC | |
| | | | | | | gGcC | NO: 662) | gC | | | | | | CAGGT | |
| | | | | | | (SEQ | | (SEQ ID | | | | | | GTC | |
| | | | | | | ID NO: | | NO: 713) | | | | | | (SEQ ID | |
| | | | | | | 513) | | | | | | | | NO: | |
| | | | | | | | | | | | | | | 813) | |
| 43 | 5 | 2.744 | L-13-R | 13 | 6_5 | gggaG | agctccg | gGGCg | chr19 | 4969006 | Promoter | KDM4B | No | No | No |
| | | | | | | gGGC | gccaat | CTCgG | | | | | Primers | Primers | Primers |
| | | | | | | GGaG | (SEQ ID | GCGTG | | | | | | | |
| | | | | | | CTGTC | NO: | gAt | | | | | | | |
| | | | | | | (SEQ | 663) | (SEQ ID | | | | | | | |
| | | | | | | ID NO: | | NO: 714) | | | | | | | |
| | | | | | | 514) | | | | | | | | | |
| 44 | 2 | 2.748 | L-11-R | 11 | 6_6 | TgACa | ggcagca | AGGtaC | chr18 | 71825292 | Exon | TIMM21 | 018- | TGGCC | 61.288 |
| | | | | | | aGGC | tgtc | TgtGGC | | | | | 071825081 | TCCCA | |
| | | | | | | GGGGt | (SEQ ID | tTGAAt | | | | | | GATTG | |
| | | | | | | cGcC | NO: 664) | (SEQ ID | | | | | | CTGGG | |
| | | | | | | (SEQ | | NO: 715) | | | | | | AT | |
| | | | | | | ID NO: | | | | | | | | (SEQ ID | |
| | | | | | | 515) | | | | | | | | NO: | |
| | | | | | | | | | | | | | | 814) | |
| 45 | 5 | 2.749 | R-20-L | 20 | 6_5 | GTcCA | tggggca | GACAG | chr14 | 77161783 | Intergenic | VASH1 | 014- | GCTTG | 61.508 |
| | | | | | | gGCtG | ctggcag | CCgCtC | | | | | 077161687 | GAGCT | |
| | | | | | | cAGaG | cagtga | tGtGTg | | | | | | GGGCC | |
| | | | | | | CCa | (SEQ ID | A (SEQ | | | | | | ATCAG | |
| | | | | | | (SEQ | NO: | ID NO: | | | | | | A (SEQ | |
| | | | | | | ID NO: | 665) | 716) | | | | | | ID NO: | |
| | | | | | | 516) | | | | | | | | 815) | |
| 46 | 2 | 2.749 | R-18-L | 18 | 6_6 | GTTCt | gggcctg | GcCAGt | chr16 | 28022533 | Intron | GSG1L | 016- | CTGTA | 61.288 |
| | | | | | | CagCa | gcctggtc | CCtGC | | | | | 028022390 | CACAC | |
| | | | | | | aAGaG | ctt (SEQ | CtCaTg | | | | | | GCCCA | |
| | | | | | | CCT | ID NO: | A (SEQ | | | | | | GATGG | |
| | | | | | | (SEQ | 666) | ID NO: | | | | | | CT | |
| | | | | | | ID NO: | | 717) | | | | | | (SEQ ID | |
| | | | | | | 517) | | | | | | | | NO: 816) | |
| 47 | 5 | 2.752 | L-14-R | 14 | 6_5 | TcACct | tcatgagc | tGGCCg | chr1 | 40236360 | Exon | OXCT2 | 001- | GCTTC | 60.908 |
| | | | | | | ctCGG | gccagg | TCCGG | | | | | 040236250 | TCCTG | |
| | | | | | | GGCT | (SEQ ID | gGTGtA | | | | | | AAGAC | |
| | | | | | | GgC | NO: 667) | g (SEQ | | | | | | CACGT | |
| | | | | | | (SEQ | | ID NO: | | | | | | TTCC | |
| | | | | | | ID NO: | | 718) | | | | | | (SEQ ID | |
| | | | | | | 518) | | | | | | | | NO: 817) | |
| 48 | 5 | 2.752 | R-14-L | 14 | 6_5 | cTaCA | cctggcg | GcCAG | chr1 | 39981146 | Intron | BMP8A | 001- | TGTTC | 62.768 |
| | | | | | | CcCCG | ctcatga | CCCCG | | | | | 039980964 | CTACG | |
| | | | | | | GAcGG | (SEQ ID | agagGT | | | | | | TGGGC | |
| | | | | | | CCa | NO: 668) | gA | | | | | | GAGAA | |
| | | | | | | (SEQ | | (SEQ ID | | | | | | CACC | |
| | | | | | | ID NO: | | NO: 719) | | | | | | (SEQ ID | |
| | | | | | | 519) | | | | | | | | NO: | |
| | | | | | | | | | | | | | | 818) | |
| 49 | 5 | 2.753 | R-20-L | 20 | 6_5 | GTgCct | ggtgcgg | GACgG | chr20 | 56328503 | Intergenic | PMEPA1 | 020- | CTGTT | 61.088 |
| | | | | | | cCtGcA | acccagt | CtCtGCt | | | | | 056328249 | TTCCA | |
| | | | | | | GGGC | gctcca | GaGTA | | | | | | CAGTG | |
| | | | | | | CT (SEQ | (SEQ ID | A (SEQ | | | | | | GCCGA | |
| | | | | | | ID NO: | NO: 669) | ID NO: | | | | | | ACC | |
| | | | | | | 520) | | 720) | | | | | | (SEQ ID | |
| | | | | | | | | | | | | | | NO: 819) | |
| 50 | 2 | 2.754 | L-15-R | 15 | 6_6 | CTACG | gaggctct | ctGCCa | chr15 | 42706924 | Exon | ZFP106 | 015- | CTGCA | 61.088 |
| | | | | | | CcGCa | cagcaca | gCCaG | | | | | 042706718 | CACTT | |
| | | | | | | GaGCa | (SEQ ID | CGTGA | | | | | | GCACA | |
| | | | | | | tTC | NO: 670) | Aa | | | | | | CACAC | |
| | | | | | | (SEQ | | (SEQ ID | | | | | | AGG | |
| | | | | | | ID NO: | | NO: | | | | | | (SEQ ID | |
| | | | | | | 521) | | 721) | | | | | | NO: | |
| | | | | | | | | | | | | | | 820) | |

| | | Reverse | Expected | | | |
|---|---|---|---|---|---|---|
| | Reverse | Melt | Product | Digestion | Digestion | |
| Ranking | Primer | Temp | Size | Size1 | Size2 | Amplicon Sequence |
| JAK2-DdCBE | | | | | | |
| 1 | CTCCA | 61.508 | 406 | 262 | 144 | GTGTGGTCAGGACAAACTGTTCCCTGAAC |
| | GCCTG | | | | | TTAAAAGGTGAAGGACAAGACCCCATATT |
| | GGCGA | | | | | ATTATCCTGTATTAAAAAAGGAAATATACA |
| | CTGAA | | | | | TATATGTACACAGACACCCCATATCACAG |
| | T (SEQ | | | | | ACAAGAAACTTCCCATAATTCAAAGGGAG |
| | ID NO: | | | | | ACCATTTCCTTATTAGCAAAGGTGCGCAT |
| | 821) | | | | | TACAGTATTTCATGACAGTTAAAAATTACA |
| | | | | | | CACCTACCACCTGTTTTGGTCAATCTTGC |
| | | | | | | TAAAAAAGACACTGAAATAGACAATTTTCT |
| | | | | | | TCTTTAAGGTAAAGACAATGTCTAATTTAG |
| | | | | | | AAAATCTTCCTCTTGAGATTGCACAGTGA |
| | | | | | | AAGGTATCAGTTAATTAAATATAAAAGCTT |
| | | | | | | ACCGTTTTTGTTTTTTTTTGAGACAGAGTT |
| | | | | | | TTATTCAGTCGCCCAGGCTGGAG (SEQ ID |
| | | | | | | NO: 320) |
| 2 | CCACT | 59.148 | 332 | 205 | 127 | GGGGCTGGGAGAAGAGAGGAATGGGAG |
| | GATAA | | | | | TTATTGATAATGGGTACAGTTTCAGTTTGA |
| | CCTAT | | | | | AAAGATGAAAAATTCTGAAGATGGATGGT |
| | GCCCA | | | | | GGTAGTGATTATACAACAATGTGAATATAC |
| | GGA | | | | | TTAATGCTGCTGAACTATACAGTTTAAAAT |
| | (SEQ ID | | | | | GGTTAAATGGTACATTTTATGTATACGTTA |
| | NO: 822) | | | | | CCATAAAAAAAAAAGTCTGGACTATAATGT |
| | | | | | | TAGAAGTAAAGATAGTAGTAATCTTATAGG |
| | | | | | | AGAAAGAAGAGGATGGTGTTCAGAAAGA |
| | | | | | | GCGTGGTGAGGGTGGGGAACTTCAAGG |
| | | | | | | GTGCTGGCAATATTCTATTCCTGGGCATA |
| | | | | | | GGTTATCAGTGG (SEQ ID NO: 321) |
| 3 | CTATG | 61.548 | 377 | 242 | 135 | GTCCACTCTGTGTTACCCAGATCTTGTAC |
| | GAGAC | | | | | AGGAAATGGAGTGTATTTAATATTGATGA |
| | TCCTT | | | | | CTCTGTATGTGTCATTTAATTGTCTCTATT |
| | CAGGT | | | | | GATTGGATTTCATCAGAGTGTGGGGCTTG |
| | CTTTTT | | | | | GACAGAGCTTTGATTAGACTGTAAGGATT |
| | TCCC | | | | | CTTTGCCGTTTTCTTTTTTCCGACACCATA |
| | (SEQ ID | | | | | TCAATGTACCTTCTGGTGTGATATCACTTT |
| | NO: | | | | | CACAATCAATATCTGAAAAAAGCTCTGCA |
| | 823) | | | | | CACAGGCCTGCAGGAAAAGAGAGAAGGC |
| | | | | | | ACTGCCTCTAAGTCCCCTTATACACATAG |
| | | | | | | ATTTAACCTCTCCAATTGAAAAAGGTTTAT |
| | | | | | | TGCATGTTTTAGAGCAGGATGGAGTGGG |
| | | | | | | AAAAAAGACCTGAAGGAGTCTCCATAG |
| | | | | | | (SEQ ID NO: 322) |
| 4 | GAGTG | 61.288 | 411 | 252 | 159 | GCCGGTTTAGGTATGGGAACCACACAAC |
| | AGACC | | | | | CTCTAAAGTGAAGGAGGCTTCTCCAGCAA |
| | CTGTC | | | | | AACCGAACGGCATATTCAGATGCTGGGT |
| | TTGGG | | | | | GAGGATTCATAATATTTAGCTATTTTGTTA |
| | GA | | | | | ATTAATATAACAGTAATTTCTTTATTCAGTC |
| | (SEQ ID | | | | | ACAGAAACATCAATGTATCTATGCTGAAG |
| | NO: 824) | | | | | CACCATAATTTTATAAGCACAGTTATTGGG |
| | | | | | | GGAAAATGTTACCTTTTTGTCCTAAAAAGA |
| | | | | | | CTCTCCACGCATGTAGCTAGGGAAAGCTA |
| | | | | | | AGGTCAGTGGCAGAGTTATATGCACACCT |
| | | | | | | CCACCACCAACATCACCACCACCACCTCC |
| | | | | | | TCCTTCATCTCTCATCCAGCTCTGCCTTA |
| | | | | | | GGCCCTTTTTTTTCTTTTCTTTTCTTTTCTT |
| | | | | | | TTTTTTTTCCCCAAGACAGGGTCTCACTC |
| | | | | | | (SEQ ID NO: 323) |
| 5 | CCTAT | 59.148 | 458 | 298 | 160 | TTACCCCTGGATCCCCTCCCTTTCTACTG |
| | CCTGG | | | | | CAAGCCACTTAAAGGAAGGAACCATATCT |
| | TAGAC | | | | | CATTCCTCTTTTCATTCTTAGCATCTGACA |
| | CTAGT | | | | | CAGTGCCAGCATGAAAAATTCCTAACAAA |
| | TGG | | | | | AGTATGCTGAATACATTAATAACCAGTACT |
| | (SEQ ID | | | | | TAATAAAAATGTGTAAAAACCGGGGTGAG |
| | NO: 825) | | | | | TTGACTGAGTACATCTATGTTAGGGCAAT |
| | | | | | | ATAAGTTCCACCAACAAGATACCAAGTCC |
| | | | | | | CCAAAGAGTGGAAGAAATAATCTTCCTGA |
| | | | | | | ATAAGAAAGATTACTTATATCTATACCTGA |
| | | | | | | TGATACATTAGTGGAGTGTTTTTCAGTTCT |
| | | | | | | TT |
| | | | | | | TTAAGATACGAAGAATTATTCTCATATTTA |
| | | | | | | AGTCAATAGGAATTAAAACACATTTTCAAA |
| | | | | | | CTGGTGGACTGCCAACAGAATCAATTCAA |
| | | | | | | TTAGCCAGTTTATCGAGTATACCAACTAG |
| | | | | | | GTCTACCAGGATAGG (SEQ ID NO: 919) |
| 6 | TAGTC | 61.508 | 329 | 202 | 127 | GGTGTGAATCCGGGAGGCAGAGTTTGCA |
| | CCAGC | | | | | GTGAGCCGAGATCGTGCCACTGCACTCC |
| | TACTC | | | | | AGCCTGGGTGACAGAGCAAGACTCCATC |
| | GGGAG | | | | | TCAACAACAACAACAACAAAAGTGTCTCC |
| | G (SEQ | | | | | CCTTCCCTGATGGAATCTGCTGCCAGGG |
| | ID NO: | | | | | AGAACCCACAGTCAGGGAAGAGGGTGTG |
| | 826) | | | | | GGATGCTGCTTCTCTCTTTACTTTTTTTTTT |
| | | | | | | TTTTTTTGATGCAGAGTCTTGCTCTGTGG |
| | | | | | | CCCAGGCTGGAATGCAGTGGCACAATCT |
| | | | | | | CGGCTCACTACAAGCTCTGCCTCCCAGAT |
| | | | | | | TCATGCCATTCTTCTGCCTCAGCCTCCCG |
| | | | | | | AGTAGCTGGGACTA (SEQ ID NO: 920) |
| 7 | GGCTC | 60.058 | 391 | 136 | 255 | GGAACAGTGATCAGTTCTGTTACCTGACC |
| | TGAAG | | | | | ATAATGGCTAAACAAAACAACCAAATTATA |
| | AAAGT | | | | | AATTAAAAGGGGTTTGAATATATAGAACAT |
| | ACGTA | | | | | TCATTTTTCAGCTACTAGCCAAAAAATACT |
| | AAAGA | | | | | ATCTCTACCTGCTATCCCATGTGTTGTGTT |
| | CCAAG | | | | | CTTTTTAGACTGTCTAATTTTGAGCTGAAC |
| | (SEQ ID | | | | | TGTTTCTTAGACTGACAGACAAGTCATAAA |
| | NO: 827) | | | | | TCAAGTGATTGCTATAAATGCTTTTTATTTA |
| | | | | | | TATAGAGAGTGTCTCAGATTTTTACCTTTC |
| | | | | | | TTTCATAAACTTGTCATAAATTTCTAATTCA |
| | | | | | | CCAATAGATGGTTGGTGTCCATTTTTCTTG |
| | | | | | | AGTCTAAGACACTTTTAAAAATTTACTTGA |
| | | | | | | CTTGGTCTTTTACGTACTTTCTTCAGAGCC |
| | | | | | | (SEQ ID NO: 324) |
| 8 | GGGTG | 59.508 | 368 | 237 | 131 | GCTACTGGGGAGCCTGAAGTGGGAGGAA |
| | CGTCT | | | | | TGCTTGGACCTGAGAGTTGGAGGCTGCA |
| | TCATG | | | | | GTGAGCCATGACTGTGTCACTGCACTCAA |
| | GTCTC | | | | | CTTAGGTGACAGAGTGAGACCCTGTCTCA |
| | (SEQ ID | | | | | AAAAAAAAAAAAAAAAGTAATAATAACAGT |
| | NO: 828) | | | | | TGTCTCTAGGTAGATGAAATTACAAGTTTG |
| | | | | | | TATGTGCTTTTCTGAAATGAACAGAAAGG |
| | | | | | | GAAAGAAAATCTCAAACCAACTATACATTT |
| | | | | | | TAAAGAGAGGTAAAGAAAGAAGAAATACC |
| | | | | | | AAGGACAGGTAGGAAAGAAAGGCTGTGG |
| | | | | | | ACATAATATTCCTTACTGCTGAGATGAAAA |
| | | | | | | CTGAATTGTAACATATCCGTTAGTTTCAGA |
| | | | | | | GACCATGAAGACGCACCC (SEQ ID NO: |
| | | | | | | 921) |
| 9 | CCCAG | 61.088 | 349 | 137 | 212 | TCCGGATTCTCCTGCTGAGGCAGGAGAA |
| | GACAG | | | | | TCGCTTGAAGCTGGGAGGCAGAGATTGC |
| | CTTTG | | | | | AGTGAGCTAAGATCAGGCCATTGCACTCC |
| | AATGT | | | | | AGCCTGGGTGACAGAGCGAGACTCTGTC |
| | GGC | | | | | TCAAAAAAACAAAACAAAACAAAAAAAAAG |
| | (SEQ ID | | | | | AGAAAGTCAGTAGCATGTAGATCAGGAGT |
| | NO: 829) | | | | | GTCCAATCTTTTGGCTCCTCTGGAAGAAG |
| | | | | | | AATTGTCTTGGGCCACACATAAAATACAC |
| | | | | | | CATCACCTAACGATAGCTGATGAGCTTAA |
| | | | | | | AAAAAATCACAGAAAAATATCATAATGTTT |
| | | | | | | TAAGAATGTTTATGAATTTTTTACAAATTTC |
| | | | | | | TGTTGGGCCACATTCAAAGCTGTCCTGG |
| | | | | | | G (SEQ ID NO: 325) |
| 10 | GCCCA | 59.258 | 461 | 288 | 173 | CCGACTGCTCTGCCTTCTGAATCATATGT |
| | TGTATT | | | | | AACCAAATCAAGTCAAACAGGTTAGAAGA |
| | GGGGC | | | | | CAACTCACACTTCAGTGTCATCTGTACTCT |
| | ATAAC | | | | | TATCTTCATGAGTGTGGGAATGTACAATC |
| | C (SEQ | | | | | CACTTTCGCTCACTATATTAATTCATTCTG |
| | ID NO: | | | | | GTTCCTCATGTACTTAACCTATTTTTATTTT |
| | 830) | | | | | TTCAGTTTGGATACAACCCAAATCCTCTCA |
| | | | | | | AGCCTTTTAAATGCAAAAAAAAAAAATAAA |
| | | | | | | TTTAAAGTATATGTAGTTAAAAATACTCAT |
| | | | | | | GTCTTTACCCATTCCTTTGAGAATTTCTGT |
| | | | | | | AGAGGCTTTCTCAAAATGCAGAGAGTGGA |
| | | | | | | GG |
| | | | | | | CAGTCATAACATATGATGCCTGCAGATTG |
| | | | | | | GGGTATCTGTCATTTAATCAAGAGAAGAA |
| | | | | | | ATAAACATTTTATGTCATTGATTTATCCATT |
| | | | | | | TTAGTTGCTATCATCTTTATAGGTTATGCC |
| | | | | | | CCAATACATGGGC (SEQ ID NO: 326) |
| 11 | GCTCT | 61.508 | 547 | 316 | 231 | GGCTAGGGGAACTCGATGTCTTTCGAAAA |
| | GGCTC | | | | | TATAGACGCTAGCCCTCCTTGAGACATTA |
| | TTGCA | | | | | TAATTCAGTTATTCATGTACAGTTGGCTAA |
| | GACCA | | | | | AAACACATACCAATCTTTTTGCTTAAGAAC |
| | C (SEQ | | | | | CACACAAGGGAAAAAATGTGGAAATTTAG |
| | ID NO: | | | | | TAAAACTCAATTTTAAGTGTCCATGAATGA |
| | 831) | | | | | CAGAGCACATTCACACTCATTCATTTACAT |
| | | | | | | ATTTTCTATGCCTGTTTTTTATGCTAAATCT |
| | | | | | | GTAGAGTTTAGTAATGGCAAAAGATACCG |
| | | | | | | TATGGTCCACAAGGCCTAAAATAGCTACT |
| | | | | | | ATCTGTACCTTACAGAAAAAAAAAAAGAG |
| | | | | | | CTGACTCTTTTTCTGACCTCTGGTCTAAGT |
| | | | | | | CAAGTCTGTCCCACAAGACACAAGGTACA |
| | | | | | | ATGCTTTTTCCACATGAGGTCAGGAAGAA |
| | | | | | | TCTCAAAACTACAGAGTCAGGCCTCACAT |
| | | | | | | TATCAATAGGACCATCCTAGCCTTCTGCA |
| | | | | | | AGCTTTCAGGCTTTAGTCAAAGCTGGCAC |
| | | | | | | ATTTTCAGTGAATCCTTAGTCCACTGGTG |
| | | | | | | GTCTGCAAGAGCCAGAGC (SEQ ID NO: |
| | | | | | | 922) |
| 12 | CAAGT | 59.038 | 532 | 304 | 228 | GTGATGGGCTTTGTTCCCAACTCTTTTAC |
| | TCCTG | | | | | ATCTCCCTACCCCTTCAACCTTTGGCCTTT |
| | GAATG | | | | | CAGCCCTTCTTTCTCTCTTCCATATTCTTT |
| | CTGCA | | | | | GGTTTGTATGTGGTTTCTCAGTTAATACAT |
| | TGTG | | | | | AGCTAATAGCTCTTATTTTTCTTATGTTTTT |
| | (SEQ ID | | | | | AACCGCTTAGGTCTATTTGGATGTAAGGG |
| | NO: 832) | | | | | TGAAAATTCATTTGATGGAAATACTTGTGT |
| | | | | | | ATATTTAAAGACCCAATTGCTCCTCTGGA |
| | | | | | | GCTTGTACTTTCAAGAATGATTAATCTGTG |
| | | | | | | TAATAAACTGGTTACTACAGTCATTACATA |
| | | | | | | TAATTTTGTGTGAATAGGCTTTTTCATTTTT |
| | | | | | | AAGAAGTTTGTCTAGCTGAGATTAGTGGT |
| | | | | | | GGATTTTCTCCCACTTCTGAAATGTTCATT |
| | | | | | | TATACTGGTTGCATTTTAAGATCATGAAAC |
| | | | | | | AATTCCAGTTACATTGTAAAAAGGATATCT |
| | | | | | | TACGAGTAATTTTATTGAACAAGTTAGAGG |
| | | | | | | CATAAGCTTAAGAGCATTTCCATGAAACA |
| | | | | | | ACACATGCAGCATTCCAGGAACTTG (SEQ |
| | | | | | | ID NO: 923) |
| 13 | CCACC | 61.508 | 368 | 222 | 146 | CAAGATAGGGCCTTGCTGTGTTGCCCAG |
| | GTGCC | | | | | GCTGGAGTGCAGTGGCACAATCACGGCT |
| | CGGCC | | | | | CACTGCAGCCTCGACTTCACTGGCTCAA |
| | TATTTT | | | | | GCAATCCTTCTACCTCAGCCTCCCAAGTG |
| | (SEQ ID | | | | | GCTGGGACTCCAGGCATGCACCACCACA |
| | NO: 833) | | | | | CCTGGCTAATTTTTTTTTTTTTTTTTGCAGA |
| | | | | | | GATAGGGTTAATTGAGCATTTTTTCCTCCT |
| | | | | | | TTCTTAACATATCAGTTATAATCATTTTTTA |
| | | | | | | AAGTTTTTTTTAGAGGTTGCCCTAGAATTT |
| | | | | | | GCAGTGTACATTTAGAGCTAATCCAAGTC |
| | | | | | | TTCTTTCAAATAACACCATGCCACTTCATG |
| | | | | | | GGTAATGCTAGTGCTTTATAATAATAAAAT |
| | | | | | | AGGCCGGGCACGGTGG (SEQ ID NO: 924) |
| 14 | CAGCC | 61.288 | 437 | 271 | 166 | CCTGGGATGGGAATCAGAACTGCCCAGT |
| | TGGGA | | | | | AAACGCATTAAGCACACCTGCCCTTCTTC |
| | TCCAG | | | | | AGTTGGTACAGTGATTGGTGCCCTATTTA |
| | TGACT | | | | | ACTTTTCCTTTTCCCTCACAGGTCTTCTGG |
| | GT | | | | | AGACTTCTGAGCAACCTGCTATACGTGTC |
| | (SEQ ID | | | | | CTGTCGTTGGTGATGTTTCCATGTCTCTG |
| | NO: 834) | | | | | TGTGATCTGCAAACCCCTCTGCTAAAAAA |
| | | | | | | GAATTCCGATTCGCATTCTTACCACTGGG |
| | | | | | | GCTGCACCTGCTCCACCTGTCTTTACCTT |
| | | | | | | GCGCACACACTGTTTGGGGAAGATGCTTT |
| | | | | | | TTCTGATACTTTCTGAATGGAAAGGCATC |
| | | | | | | ATAAGAAGGACATATTTGTGAAGTGCTTG |
| | | | | | | AAAGTCCT |
| | | | | | | CTCATACTTTCCAACGCAGAAGGACTAAA |
| | | | | | | GTCTTTGTCAAAGTTAAGGTAAAATACATC |
| | | | | | | ACAGTCACTGGATCCCAGGCTG (SEQ ID |
| | | | | | | NO: 925) |
| 15 | CCAGC | 61.508 | 526 | 303 | 223 | CAGGTATTATGTTAGGCCATGGGGATACA |
| | CTCCT | | | | | AAGATGATTAAAACCACTTCCTCCCCTCA |
| | GACAA | | | | | AAGAATTCTCATGATGGCAGAAATGGGGC |
| | CAGAG | | | | | ATATGCAAACAAGTAGTCAAACAAGCAAT |
| | C (SEQ | | | | | TCCAAACATATAAGAGCTATGATAGATGA |
| | ID NO: | | | | | ACATTCAGAGTGCTTTAAAACCACTGAGA |
| | 835) | | | | | AAGGAACAATCAATCTGTCTCCTTTCCATA |
| | | | | | | GAGTAGACATATGAGCTGAGACTTGACAG |
| | | | | | | ATAATTAGGAGTTCACTGGTAGGCATGGG |
| | | | | | | ACACTGTAGAAAGAACTCTGAAAAAGACA |
| | | | | | | TTCCAGTTATAGAAAATGTAGAGACAGGG |
| | | | | | | GGAATAAAAACACATGGAAAGGAGATTAA |
| | | | | | | AATTGCTAACACTTAGGTTTTCATCATCAA |
| | | | | | | TCTTCCCCTCAGAATTACTATTGTGATCAT |
| | | | | | | AAAATATTATTACTATTAGAAGTAGTAATA |
| | | | | | | GTAGTAGCAAGGCTTACATTTATTCATCAT |
| | | | | | | CTACTATGTACAAAGCATTTTTTTTGACAG |
| | | | | | | AGTCTTGCTCTGTTGTCAGGAGGCTGG |
| | | | | | | (SEQ ID NO: 926) |
| 16 | GAAAC | 61.508 | 395 | 270 | 125 | CGTTCTCATGGACCTGCCCAGTTATTTTG |
| | AGGGA | | | | | GTTCTAAGCTAGAATCAATCTGCTAGATG |
| | GGGGG | | | | | GTTTACCATGGCAACACAAGGTGAAGCA |
| | TGCAG | | | | | GAAACCCTGGGACAGGAGAGTCAAGCGC |
| | T (SEQ | | | | | TGTGGCCAGGACACCTAAGGCTTAGAGG |
| | ID NO: | | | | | GTTTTCCAGTAGAACCTTTCCATATCTTCC |
| | 836) | | | | | TTTGTAAAAGCTAAGGGTAGGAGAGGTGT |
| | | | | | | GTGGGAGAACCACAGCCAGCAACAAAGG |
| | | | | | | GGGTCCTTTTTGCTAAAAGAGACTCAGCC |
| | | | | | | AGTTGCTTTGTCAGGACAAGAGGGAAGG |
| | | | | | | AGAGTAGGGATGGGCTAGTAAAGACAAA |
| | | | | | | CTTCACACTGACTGTGAAACTCAGACTCA |
| | | | | | | TCCTTGTCTAGTCCACTGTGGCTAAACGA |
| | | | | | | ACACTGCACCCCCTCCCTGTTTC (SEQ ID |
| | | | | | | NO: 927) |
| 17 | GTGTG | 59.038 | 477 | 321 | 156 | CTATCAGTGAAATCTTTGCCCATTCTGTTT |
| | AGCAA | | | | | GCAAATACAGTTTTTGTGCTCTTTCTAGAA |
| | CTAGA | | | | | GTTTTATATTTTTGTAGTTTACATTGATTTA |
| | CAAAG | | | | | TATAATTTGCTGCAATTTAATTTTTCTTGTG |
| | GAGG | | | | | ATGTGAGAAAAACATAAAGTTTCCACCCC |
| | (SEQ ID | | | | | TTATCCTAAATTTTAAAGTTGTTTGAGCAC |
| | NO: | | | | | CATTTGTTGAAAAAACAATCCTTCCCCCAC |
| | 837) | | | | | TAGGATCTTTGTTGAAAATCAATTGATAAA |
| | | | | | | ATATATGTATATTTATTTCTGGTTTGTATAT |
| | | | | | | TTTGTTCCATTGATAGAGATATTTATCCTT |
| | | | | | | CTGTCTTTAATTCACCTTCATTTGAAGAGT |
| | | | | | | ATTTTTTATGAATATAGAATTTTAGATTGAT |
| | | | | | | TTACTTTTTCTTTCAGAATTTAAATATGCCA |
| | | | | | | CTCAATTGTCTTCTGGTTGCATAAATTCTG |
| | | | | | | ACAAAAACTCAATGATTATTTTACAATTTC |
| | | | | | | CTCCTTTGTCTAGTTGCTCACAC (SEQ ID |
| | | | | | | NO: 928) |
| 18 | GGGGA | 60.058 | 440 | 315 | 125 | GGGCTTCCCTTTCTTCCACTAGATTAAGTT |
| | GTTAG | | | | | CAGAAGCCACTCATTTATATTTAAAATATC |
| | ATATAA | | | | | ATATTATGTAAAATGTTAATCCAATAACCT |
| | AAAGA | | | | | AAGTTTGAAATATTATTTTTTTCCTCTCTTG |
| | CTAGG | | | | | CACTTTGTTAATATAGATGTTTCATTGTTCT |
| | GGAG | | | | | GTAGGGCCTCTATCTTCCCTTGAGCTGTT |
| | (SEQ ID | | | | | TTTGAGTAACTTGAATGCTTTCCATAAAAC |
| | NO: 838) | | | | | ATCATTTTTACTATCACTAGAAATTTAATCT |
| | | | | | | CAAGCTATATTTGTGTTTTTCTCAGTGTAA |
| | | | | | | TAAGCCACTTCATTGCTAAAACAAACTTTG |
| | | | | | | TATTATTGAAAATTTCCAACAGGTAAAGAG |
| | | | | | | AACAGTATATAAACCTCCATGAATGAATTA |
| | | | | | | TGCAGATTAAACAGTTATCAACATTTTTCT |
| | | | | | | CATTATTGTTCCATTTAACTCCCCTAGTCT |
| | | | | | | TTTTATATCTAACTCCCC (SEQ ID NO: 929) |
| 19 | GCCTG | 60.908 | 454 | 172 | 282 | CCCCTCTCCCAAAAGGAGAGCTTTATGAT |
| | GGTAA | | | | | GACATGTGATGAAGAATTTTATGCTGTTCT |
| | AAGAG | | | | | GCTAAAATCACATTTACCATTTCGCATATT |
| | CGAAA | | | | | GGGCTTCAATTTTTAATTTACTCGTTTTAAT |
| | CTCC | | | | | AAGCCTCATTACTTCCAAGGTTATTTATAC |
| | (SEQ ID | | | | | AGTGTTTAACTCCTACTGGGTGTTTCTTTT |
| | NO: 839) | | | | | TCTGAGTCTTTTTTAGCTTTTAAGTAATGG |
| | | | | | | CTGTGATTATATAATTAAAGCAAACTACAG |
| | | | | | | CAGATTACATTAAGAAGTTCACTTTTTTTG |
| | | | | | | GAAGAAGGAAGTTTGCTCTTTGTTCACTTT |
| | | | | | | CATATTATTCATTTTTCATAATTTAATATTA |
| | | | | | | CCAAAGATAGTGCATAAATATTATAATTAG |
| | | | | | | CATATTTGTTGTTTTTCTTGAATGGATATT |
| | | | | | | GTGATTTACCTTTGTTTTTGTTTTTTGTTTT |
| | | | | | | TTTGAGACGGAGTTTCGCTCTTTTACCCA |
| | | | | | | GGC (SEQ ID NO: 930) |
| 20 | TGAGC | 61.508 | 520 | 327 | 193 | GCAAGTGATACAACCCCAAGCCACTAACA |
| | TGCCT | | | | | TATATGGCAAGAAGGAGAAGCAGAAGAA |
| | TCCGC | | | | | AACCAGGGAAAAGAAAAAGCAGATACTCA |
| | CTCTG | | | | | TTGCCCTAAGAAAAATAATTTTGTAATCAC |
| | T (SEQ | | | | | AAAATTAAATTACTCTGCTGTGAATCCCAG |
| | ID NO: | | | | | TGAAGAAATGGCCTTGTTGGAATGGTTTG |
| | 840) | | | | | ATGTGCTCTCACAGGCATCTCAAATGGGA |
| | | | | | | AAACATACCCACAGGAAAAATTTGGAAGG |
| | | | | | | CAAACAGTAAAAACTTTCACATTAATCTGG |
| | | | | | | AAAACAAAAGAGTGGCCATTTATACCTTAT |
| | | | | | | GCCTAACTCTAAAAAACTCTTAAGTGCTTT |
| | | | | | | GCAGTTTATTTTTGGAAAAGAGAGTAGGA |
| | | | | | | GAAAGAAGAAAGTGGGGCAGAATAACAG |
| | | | | | | GGAGAGAGGGAAAGAAACAGATTTTTTTT |
| | | | | | | TTCCTTTTGGTCCAAAAGATGTTAAAAAGA |
| | | | | | | ATGTTACTCTAAGGGAAGAGTGGTATTTT |
| | | | | | | CTACCTCATTGGTGAATCTTGGGCTAGTC |
| | | | | | | CTACAGAGGCGGAAGGCAGCTCA (SEQ ID |
| | | | | | | NO: 931) |
| 21 | CACCT | 61.088 | 421 | 292 | 129 | CCAGCCTTCAGTTTGCCAAGGGGTTGGC |
| | GTAGT | | | | | ATTAATCAAATCGTTTTCTGTAATTCCTGC |
| | CCCAC | | | | | CATGGAGCCTTAGTTTCCTTTTATAATCTG |
| | TCAAG | | | | | GTTCTTCGGCCTTCATGCAGGTTCAATAA |
| | AGC | | | | | AAGCTACCTGAAATCTTTCTAATAAATTTC |
| | (SEQ ID | | | | | ATATTTTCTAGATAGAGCCTGTTTCTGTTG |
| | NO: 841) | | | | | CCTGTAGCCAAGGAACCCTAGTTGATTTT |
| | | | | | | TTTTCTTTAATTTACACAACATTGTTATTGA |
| | | | | | | ATAGTTTCTGTCTGCCAGGCAATGTACTA |
| | | | | | | AACGCTAAACTCTTTACCTGCATTATCCTA |
| | | | | | | TGCAATTTTTTTTTGAGATAGGTTCTGTTG |
| | | | | | | CCCAGGCTGGAGTGCAGTGGTATGATCA |
| | | | | | | AATGCAGCTTCTGTTTCTGGGCTTAAGCA |
| | | | | | | ATCCTCCCACCTCAGGCTCTTGAGTGGG |
| | | | | | | ACTACAGGTG (SEQ ID NO: 932) |
| 22 | CTCTT | 60.058 | 463 | 279 | 184 | GCACACCAGAGGAGAGCTGCTTGCTTAG |
| | GCTAT | | | | | TAAAAATTATGACATTCACAGCAAAACAGC |
| | ATCTT | | | | | AATCCTCTAAGATCATTTTTTGTGTCCATA |
| | GGAAG | | | | | GAGATGAAGCTATGGAGTTGGTGTGCAT |
| | GTTAT | | | | | GTGACTTGTGAATCACCAGAAAATCTTCA |
| | GTGGC | | | | | GTGTTTTAATTTCAAAAACACCTTGACTCT |
| | (SEQ ID | | | | | CTCTGGGGCAAGGGGCGTGTCACTGATC |
| | NO: | | | | | GTCTCTGTGCCTTAATTTATTTATCTTAAG |
| | 842) | | | | | CAGAACAATTTTTAAATAACTACCTTCTTTA |
| | | | | | | CCACTTAGAGACATGTGAGGTCCTGTCAA |
| | | | | | | GTATTTTTCAGAAAGATACTAATTAAGCAT |
| | | | | | | GCATTTTACGGTTACTAATTTAGTACGGTA |
| | | | | | | AATGGATTATACTTACAATAACTAGGAAG |
| | | | | | | GGCTCAAAGTATTTATACAAAATCAGTCAA |
| | | | | | | AATGGGATAAAATGTCAAGTAGCCACATA |
| | | | | | | ACCTTCCAAGATATAGCAAGAG (SEQ ID |
| | | | | | | NO: 933) |
| 23 | CGGCT | 61.288 | 434 | 145 | 289 | CCCAGTCTTGGGCAGCCCTTTATAGCAGT |
| | TACCC | | | | | GTGAGAACAGACTAATACACCATTCTTAC |
| | ATTCC | | | | | CACTTTCTAACAGTTCTTGTTTTCACTTTCT |
| | TCCAT | | | | | GTATGACTTTTCAACCCATTTTCCCATCTA |
| | GC | | | | | CTTCTACCTTCACCTTTTCACTTATGAATT |
| | (SEQ ID | | | | | CATTCTTCAGTCTTTTTCAGATACGAAGTC |
| | NO: 843) | | | | | TTCTTACCTTTCTCTTATCAATTTCATTTTT |
| | | | | | | TTATATTCCCTGATGTCTTACATGTACTTTT |
| | | | | | | GTGAGCATATTTGTGTGTGTTCATATTTAT |
| | | | | | | ATCGTAGTGTCTGTATATATTCATAAATAT |
| | | | | | | GTCTGCATATGTACATTTTTATATCTATCT |
| | | | | | | GTGTGCATTATAGACTGTATATGTATCTTT |
| | | | | | | CGGTTGTGGCTAAAACTGAGCAAGAAGTT |
| | | | | | | TATTTCATTAGTTACAGCTTCTGCATGGAG |
| | | | | | | GAATGGGTAAGCCG (SEQ ID NO: 934) |
| 24 | AGTGG | 61.508 | 385 | 136 | 249 | GCATTTAGGACAGAGAGCGAAGCATATGT |
| | CCCAA | | | | | AAGGGCCCCAAGGCAGGTAGAAGCATGG |
| | GCTGG | | | | | CAGCCAGGGGGGACTGAGGGAAAGACA |
| | CACCA | | | | | TTGTTGCTTTTGAACACAGTGAACTAAAAA |
| | T (SEQ | | | | | AAAGTGTGGAATACTAGAGGCAAAAAAGG |
| | ID NO: | | | | | TAGGTAAAGACTGGATCATACAGTTATAA |
| | 844) | | | | | AGATTTTCCCCTAAAAACAAATACAAGCTA |
| | | | | | | CTGAAGTATTTTAAGTTGAGATGGGGTTG |
| | | | | | | AAGGACCAGATCAAGTTTATGTTAAAAAAA |
| | | | | | | AAAAAAACCACTCTGGCTGAAGTATGGAT |
| | | | | | | ATCAGATTTGAGGGGAGGACAGTGTGAA |
| | | | | | | TGTGAGTAGATCAATTAGGAGTTCGTTAT |
| | | | | | | AGTAGTTGTGCCAAGAGATGGTGCCAGC |
| | | | | | | TTGGGCCACT (SEQ ID NO: 935) |
| 25 | CAGGC | 61.508 | 531 | 206 | 325 | GGCCCTTGCTATGCCAGGGAAGTTTAAAA |
| | TGAGG | | | | | ATCTTGAGAGGAGTAAGTCTGTTATAACA |
| | GATAG | | | | | GATATTCTTTCACTCTTCCATATAATTGCC |
| | CTGGG | | | | | CTCCATTTACTGCTTCCTCTTTCCTTTCTC |
| | A (SEQ | | | | | TTCTCTTTTCCCTTTTATCTTTTTCAATGTT |
| | ID NO: | | | | | TTCTCATAACTTTTTCTTTTCCTTTCCATTC |
| | 845) | | | | | TACTGGCATTACATCTTAGCTCCTGTGGT |
| | | | | | | GGGTATGCAGTATTTGTTTCAGTTTAAACT |
| | | | | | | ACATGCAAAGCATTTCAAAAAATACTGTGA |
| | | | | | | TGAAACATACTGCTTGATAATTTAATTTTG |
| | | | | | | TTCTAAAAATAGTTATTATTTGGGGACATT |
| | | | | | | TTAAATAAAGCTAGTCCTAGTGCTAATTTC |
| | | | | | | TTTATATTTGTGAAATAACTTTGATAAGAAT |
| | | | | | | ATCTTATTTTTAATAATACACTCTAACAAAT |
| | | | | | | AAACACTTTAATGAAGAAAAATAATAGGTT |
| | | | | | | AATTGATGTCCCAAGGCTTCAAAATTACTT |
| | | | | | | AGTCACTACATATGAAATAGAACTTAGATT |
| | | | | | | CCCAGCTATCCCTCAGCCTG (SEQ ID NO: |
| | | | | | | 936) |
| 26 | GCCTC | 59.508 | 395 | 243 | 152 | CACCATTCTCCCTGCCTCAGCTTCCCGAG |
| | CCCAG | | | | | TAGCTGGGACTACAGGCACCCGCCACCA |
| | AGAAG | | | | | TGCCCAGCTAATTTTTTGTATTTTTAGTAG |
| | ACAAC | | | | | AGACAGGGATTCACCATGTTAGCCAGGTT |
| | (SEQ ID | | | | | GGTCTCGATCTCCTGATCTTGTGATCCGC |
| | NO: 846) | | | | | CCGCCTCGGCCTCCCAAAGTGCTAGGAT |
| | | | | | | TACAGGCGTGAGCCGCCGCGTCTGGCC |
| | | | | | | CCAAATGTGAATTTTTATTGCAACTATCCT |
| | | | | | | TTCCCTGTTCCATCATTCGGAGATTTTTTT |
| | | | | | | TTAGAATATAAATTGCTGGTCCATAAAAAC |
| | | | | | | CTCTCTCAGACCTAAGGGAAGATACTGCA |
| | | | | | | TCTGGGCCAGGGATCTTGAACTTTGAGCT |
| | | | | | | GATTAAAATGATTGGATGAATTGCTAGGTT |
| | | | | | | GTCTTCTCTGGGGAGGC (SEQ ID NO: 937) |
| 27 | GGAAA | 61.288 | 343 | 132 | 211 | CAGGAGGCTGAGGTGGGTGAATCATTTC |
| | GGGCC | | | | | AGCCCGGGAGATCAAGAATGCAGTGAGA |
| | TCCAG | | | | | TACGATCACACACTGCACTCCAGTCTGGG |
| | AAAGG | | | | | CAACAGAGCAAGGCCCTGTCTCAAAAAAA |
| | AC | | | | | CAAAACAAATAAAAAACAAAAATAGTGGG |
| | (SEQ ID | | | | | TGAACAGAGTAGTAAACCAATGGCCAGTGT |
| | NO: 847) | | | | | CAAGCTTGCCACATAGGAGA |
| | | | | | | TGAAGAAGGACCACCCACTGCCCCATAAT |
| | | | | | | TCAGAACTAGGGCCAGGGGTTAGAGCCC |
| | | | | | | TCTCCACAGTCTACTGGACATAACCTAAG |
| | | | | | | CAATGACTGCAGGGCACGGAAGATGGCA |
| | | | | | | TAGAGGATGTGTTTTGTCCTTTCTGGAGG |
| | | | | | | CCCTTTCC (SEQ ID NO: 938) |
| 28 | TCCTC | 59.508 | 373 | 130 | 243 | GCTAGCATTTGCACTCCTGGGCAATTATC |
| | CCTTC | | | | | CCAAAGAAATGAAAACTTAGGTCCACACA |
| | CCTAC | | | | | AAAACTTATACACAAATGTTCATAGTAGTA |
| | CTTCC | | | | | TTATTAGAATAGCTAAAAATTAAAAACAAT |
| | (SEQ ID | | | | | ATGCATGTCCTTCAACAGGTGAATACAGT |
| | NO: | | | | | GGTATATTCATAATATGAAACATTATTTATA |
| | 848) | | | | | CTGAAAAGAATGAACTATTGATACATACAA |
| | | | | | | CAAAACTGATGGATCTCAAGGGCATTATG |
| | | | | | | GCACATGAAAAAAGCCAATTTCAAAGGGT |
| | | | | | | TATATACTGCAAGATTCCAGTTAGAAAACA |
| | | | | | | TTCTCAAAATGACAAAATTGAACACAGATT |
| | | | | | | AGTGGTTGCTAGGGGTTAAGGATGTGAG |
| | | | | | | GAAGGTAGGGAAGGGAGGA (SEQ ID NO: |
| | | | | | | 939) |
| 29 | CAGGA | 61.508 | 438 | 312 | 126 | CACCTGTCACCACGCCTAGCTAATTTTTTT |
| | GGCTG | | | | | TTTTTAAAGATCATACTGCTTCTGTCCCTT |
| | ACGTA | | | | | TTTTTTTATTATTATTATACTTTAAGTTCTAG |
| | GCAGG | | | | | GGTACATGTGCACAATGTGCAGGTTTGTT |
| | A (SEQ | | | | | ACATATGTATACATGTGCCATGTTGGTGT |
| | ID NO: | | | | | ACCCATTAACTCGTCATTTACTTTAGGTAT |
| | 849) | | | | | TTCTCATAATGCTATCCCTCCCCCCTCCC |
| | | | | | | CCTAATTTTTTGTATTTTTACTAGAGATGG |
| | | | | | | GGTTTCACCATGTTGGCCAGGCTGGTCTT |
| | | | | | | GAACTCCTGACCTCAAGTGATCCACCTGT |
| | | | | | | CTTGGCCTCCGAATTTTTTTGTTGTGTTTT |
| | | | | | | TTTGAGACAGGGTCTCATTCTGTTGCCCA |
| | | | | | | GACTGGAGTGCGGTGGTGCAATCATGGC |
| | | | | | | TCACTGCAGCCTTGACTTCCTGGGCTCCA |
| | | | | | | GGGATCCTGCTACGTCAGCCTCCTG (SEQ |
| | | | | | | ID NO: 940) |
| 30 | GGTAT | 59.758 | 467 | 290 | 177 | GTACAGAGTAACCTATAAGTGTGACATAC |
| | CAGAG | | | | | TTTTGCTTTCAGTAGTTTCATGTAAAGAAA |
| | AATCTT | | | | | AAAACTTGAAAATAGATTACTATTAACCTG |
| | GTATTT | | | | | AGGACCCATGGGAATAACAGACACTGGG |
| | ATGGC | | | | | GAGGTAGGGTGGGGAGCGGGAACAAGA |
| | ATCAG | | | | | GCTGAAAAACTACCTACTGTGGACTCTGC |
| | G (SEQ | | | | | TCACTACCTGGGTGACAGGATCATCCATA |
| | ID NO: | | | | | CCCTAAACCTCAACGTCACACAGTACACC |
| | 850) | | | | | CAGCTAACAAACCTGCCCATGTGTTCCCT |
| | | | | | | GAATCTAAAAAAGAATCAAAATAATTGTTT |
| | | | | | | AAAAAAAGAAAAAGACAATAGTATTACCCA |
| | | | | | | TGGGACAAAATTTGTACTATTAGCAAGAAT |
| | | | | | | CATTTGTGTCTCATTTAGAAACAATTTGAC |
| | | | | | | TTTTGTTCCATTGTTTAAACTTTGACAAAAA |
| | | | | | | TGGTTTTGAATAGATCTTTATAACCTGATG |
| | | | | | | CCATAAATACAAGATTCTCTGATACC (SEQ |
| | | | | | | ID NO: 941) |
| 31 | GGAAA | 61.088 | 475 | 327 | 148 | GGGGATTCAGAGATTGTTAAAGAACACAA |
| | AAGCC | | | | | GGTAAACACAATCATTTGAAATTTAAGATA |
| | GCAGG | | | | | TGTTTTTAAATTTCAAGAATAAGTTTATTTC |
| | AAGCT | | | | | AGACATTTCTTTCACTTTATCAAATCTCTTA |
| | GCA | | | | | ATCAAAAATGAAACCCCAGATACCATATTT |
| | (SEQ ID | | | | | ATGTTATTTTGAGGTTGTGTTAATAAATAT |
| | NO: 851) | | | | | AGGTGTTCTTTTTATGTTAAGTAGGAATCC |
| | | | | | | TTAGCAACTTAACTAGTAGTGCTTGTAAAC |
| | | | | | | AGACAACAACAACAAATCCTAAAAACCAG |
| | | | | | | ACATCAGTGAAAACAAACCAACAACCAAA |
| | | | | | | AATTACTATCTGTACCTTTAAATTTGTAAG |
| | | | | | | CATTTTTTAGAGTTTGTTTGATAAACTTAG |
| | | | | | | GTTGGTTTTAAAATTTAGCTCAGTGACTCA |
| | | | | | | GTTGCAGCTGTTCCAAATAATTGGTCATG |
| | | | | | | AGCTTTTTATACTAGCTGGCTTAATCTAAC |
| | | | | | | AGAGTGCAGCTTCCTGCGGCTTTTTCC |
| | | | | | | (SEQ ID NO: 942) |
| 32 | GTGGT | 59.668 | 409 | 248 | 161 | GCACCTTGTCTATTTCTTAGAGTTGTTGAG |
| | ACAAA | | | | | CAGAAGAGTCTGTATAGGTGACAGAAAGA |
| | TGAGT | | | | | GGACTTTAAAAAAATGTATAAAATGAATTT |
| | CAACA | | | | | GAAGAGTGTGATAATATGTAAAGAAACTT |
| | TTTTGT | | | | | ATAATCAAGAGACATAAAAATAAAAAGTAG |
| | CATGT | | | | | TCAAAAATTGAGAAGATATTTTAGCCTTTA |
| | CTG | | | | | TGTCCTTACCCTTAGAGTAAGATATCTTTC |
| | (SEQ ID | | | | | ATTATACCTATCTATGCTTCTCTATTTACCT |
| | NO: | | | | | ATGTATATATACACATTCATATACTTTTTCA |
| | 852) | | | | | GTTGTATCATGTAAGTTCATATGGTCTTTC |
| | | | | | | TGAAATAAAAAGCCAAAACCAAACAGTAG |
| | | | | | | AACATAATAATTGACTTTATGCTGTTTTTCT |
| | | | | | | CTGAATGACAATATACAGACATGACAAAA |
| | | | | | | TGTTGACTCATTTGTACCAC (SEQ ID NO: |
| | | | | | | 943) |
| 33 | CACGG | 61.508 | 342 | 127 | 215 | AGTTTGCGTCTGAGCCTGGCCCTCCAGC |
| | GGCAC | | | | | CATGGCCCAGCCCAGCCCAGCTCTGCTC |
| | CTCAC | | | | | CAACGCCGTCCCCAGCCCTACCCCCTCT |
| | CAACA | | | | | TCCTCCTTCCCATCATCCTATTTTCTTTAAT |
| | A (SEQ | | | | | GAGCCAGCTCTTTGATAACTGCAGTGTGT |
| | ID NO: | | | | | TTTTTAGGAACTAAACAAACTGAAGCTGC |
| | 853) | | | | | AGGGTATTCTTTTTTTTAACAAAAGAGCTC |
| | | | | | | ATTGAATAAGCAATGGGTTTTTCTGCAACT |
| | | | | | | ATTTTTAAAGATGGAGCTGCAGGCTAGGC |
| | | | | | | TGTGAGGAAAATCACAGAGAGTGCTCTCC |
| | | | | | | TCAAGGTCAGGGCATTGAGGGAAAAATC |
| | | | | | | CATTGTTGGTGAGGTGCCCCGTG (SEQ ID |
| | | | | | | NO: 944) |
| 34 | CCTTG | 59.148 | 423 | 291 | 132 | CTGAATGGTGAGGGCTCCCTTAAATAATG |
| | CCACA | | | | | TAACAGTATTGTTATTCAGTTCAAATGTAA |
| | CTCAT | | | | | TTTAATTCAGCTACAATATGAATTAAGTAG |
| | TAATG | | | | | TTTTGGGGAACTAGCCTGGAAACTAACCA |
| | CCC | | | | | TTGACAAAACTGTTCAGATTGGAACAAAAT |
| | (SEQ ID | | | | | TCTACAGCATTTTTGAACTTTTAAGGGTCA |
| | NO: 854) | | | | | ACATTTGAAAGTTTGATGCTAAATGTTGAC |
| | | | | | | TTTCACAAATGAGAGAAAAATGCAAGAAT |
| | | | | | | ATCCAGGGTAAGTATTGGGAATTAGCTAC |
| | | | | | | AAATGACTTTCCCTTTCCCTGAAAGATAAC |
| | | | | | | AATGCATAGTTTTGTATAGAGCCCACTTTA |
| | | | | | | CAGAGTATGATAGTGAAGATATGTTTATCT |
| | | | | | | TGCTGCTTTCATTGCAGCTTTAAAGGACC |
| | | | | | | ATAACTAATTTTAAGGGGCATTAATGAGTG |
| | | | | | | TGGCAAGG (SEQ ID NO: 945) |
| 35 | CGTTC | 60.178 | 490 | 204 | 286 | GTCTCCCAGGGGAAGAATCATAGAACAG |
| | ATGTG | | | | | TGGGGAAGAAATGCATTATTCAATAAATG |
| | TTTTCA | | | | | GTGCTGGAAAGATAGTTTAGCAATTTGGA |
| | GCGGC | | | | | AAAAAAATCAATTTCAAGTCTCATCTTACT |
| | TATGTT | | | | | GCACACTAAAATAAATTTTAATGGACTAAA |
| | TC | | | | | GAACCAAAATGATATAAAAAAGACTATAAA |
| | (SEQ ID | | | | | AGCACAAAAAAACTCTACTGGGGAATACT |
| | NO: | | | | | TGGTAAGGTTAAAAGAGTTGAACAAAATC |
| | 855) | | | | | GTAAAAGAAAAGTTACAAATTTTATTAAAT |
| | | | | | | AACATTTAAAATTTCTGTCTGTAGCTCTTC |
| | | | | | | CTGTGTCTACTCAACAAAAATCAAAATAGT |
| | | | | | | TATGAAAGAGTATAAACACTACCACAATTG |
| | | | | | | GAAGACAGAATAGTTCTATTAACTCTATGC |
| | | | | | | ATTTGTCAAAACTCATAGAACTGTAAAAGT |
| | | | | | | AAAAACTTTACATAAGCTAAAAAATAAATC |
| | | | | | | ACAAAAAGGATATAAAAGAAACATAGCCG |
| | | | | | | CTGAAAACACATGAACG (SEQ ID NO: 946) |
| 36 | CACAA | 60.438 | 452 | 133 | 319 | CAGGTCCACAAACCCCTCCCATGGCTCTA |
| | ACGTT | | | | | GGGGAGAATCCTTCCTTGCCTTTTCCAGC |
| | CACTC | | | | | TTCTAGCAACTCCTGGGATACTTTAGCTT |
| | CACAG | | | | | GTCACTGCATAACTCCAACCTTTGCCTCT |
| | CAACA | | | | | GTCTTCACCATATTTCTATATGTAAAGGCT |
| | TC | | | | | TTTTTTGGATTTAGAAGACACCTGAATCCA |
| | (SEQ ID | | | | | CGATGATCTCATCTCAATCCTTATCTTAAA |
| | NO: 856) | | | | | TCTGCTATGATCCTATTTCCAAGTAAGGTT |
| | | | | | | ACATTCTGAAGTTCCGGTGGCCAGGTATC |
| | | | | | | TTGATAGGACACTATTCAACCCACTGAAA |
| | | | | | | G |
| | | | | | | TCCTTATTTACCCCTCATGTATTGTTATTCT |
| | | | | | | CCCTGCTTAAATTTTAGGATCAGTAACTAC |
| | | | | | | TCATCCAGATGTTGACTCTCATGTTCTAAT |
| | | | | | | TGAATTTAAACAAAAAGTTACTACTACAAT |
| | | | | | | GAATGTTTTGATGTTGCTGTGGAGTGAAC |
| | | | | | | GTTTGTG (SEQ ID NO: 947) |
| 37 | GCCTA | 59.378 | 480 | 317 | 163 | TCACCCAGGGTCACACGGCTAGTTAGTC |
| | CCTGA | | | | | ACAAAGTGAATCTAGAACACAAAGTCTCC |
| | AAGTT | | | | | TTTCCTCCAATCCAGAGCACTTTCCACTA |
| | CCCTG | | | | | CATCACAAGGGAAGAGCAAAAAGAAAAAA |
| | G (SEQ | | | | | TAACATCTAATTTTACCAGAAAATAAAATA |
| | ID NO: | | | | | CAGGCAGGCCTGAACCACAGCCAGAGTT |
| | 857) | | | | | AAATGTGGAGCATACCTACCTCCAAACCC |
| | | | | | | AGCTGGAACCTCAAAGGTTAGTATATATTT |
| | | | | | | TAGACTCTTCAAAAATATTTTAATCACATG |
| | | | | | | TGAATTTGAGGAAAGCTCTGATTTTAAAAA |
| | | | | | | CTCAAAAACACTCAGTAGTCACACAGCAT |
| | | | | | | AAAGTTATGACAGTATTAGCTGGATAATAA |
| | | | | | | ATTTGTAGACCACAAACTGTCCATCCAGA |
| | | | | | | CAATTTACACTTTAAAAACATCCAACCTTC |
| | | | | | | TTAGAATTAGTTTGTAAGTCAGGCAAAGA |
| | | | | | | GGACAGATTTTTCTATATTACCAGGGAAC |
| | | | | | | TTTCAGGTAGGC (SEQ ID NO: 948) |
| 38 | GACTG | 59.038 | 444 | 159 | 285 | GGAGGGGGCTGTCTTTCTGGTTCACTAG |
| | GGATC | | | | | CTGTGTCTTCCTCATGTGGTGGAAGGGG |
| | TTAAA | | | | | TAAGGCAGCCCTCTGGGGCCTCTTTTATA |
| | CATCG | | | | | AGACACTATCCTCATGATCTAACCACTTC |
| | CCCT | | | | | CTAAAGACCCCACCTCCTAATACCATCTTT |
| | (SEQ ID | | | | | ACCTTGGAGATTAGGATTTCAACATATGA |
| | NO: 858) | | | | | ATTTTTTTCAGAATGCAAATATTGAAACCA |
| | | | | | | TAGCACAATCCCACAGATAGACAGGAGAT |
| | | | | | | AGATACCTCAACTAACTGGTGTTGACCAT |
| | | | | | | TATTTGATTGGTCCATACTGTAGGACCAC |
| | | | | | | TCCATACTATAGGACTCTTGCTTTGTACTG |
| | | | | | | TAATTGGACTCTTCAGGAAGAACAGTAAC |
| | | | | | | AATTATTTTAAAGATTAAAGGGTGTCTGTT |
| | | | | | | TATTTAGTATGTTAGTTACAATGCACTGTC |
| | | | | | | TGTATGTATTTAGGGGGATGTTTAAGATC |
| | | | | | | CCAGTC (SEQ ID NO: 949) |
| 39 | CTCGG | 59.508 | 457 | 187 | 270 | CTCAGCCATGTCAAGCAAATCATTCAAGC |
| | AGATT | | | | | AAAATCTAGCTGAAAAGTCTGAAACATTCT |
| | CTGGT | | | | | TAAAAGCTTTGTTGTTATTCTAAGTCAGCC |
| | TGGCC | | | | | AAAATCCTGGTATCCCTCTTCCAGATAAA |
| | (SEQ ID | | | | | GAGCTCCCACTGAGAATTGTAGTCTATGG |
| | NO: 859) | | | | | ATTTTACCTTGACTGCAATTGTCTTTCCTT |
| | | | | | | CCTATCTGCTTGTTGTTTGTAGGTTCTTTT |
| | | | | | | TTTGTTTTTCTCAAATGCTAGTGATATTTTG |
| | | | | | | TTTACAGATTCTAAAAGCAATGCAAAATTC |
| | | | | | | TGTTGGCTTTATTTTCAGCAGAGTTAAAAC |
| | | | | | | TGATTTCATCATATTATCAGTATGTCATCTT |
| | | | | | | TATATTTATGACTGACATCTGCTATTCCAG |
| | | | | | | TGTTTATTGGAGACTTGTGAATGAATCTGT |
| | | | | | | CCAGGACACTTGTCAGTTCCTACCTGAAT |
| | | | | | | CTCTTACCTATTGAGATTTGGCCAACCAG |
| | | | | | | AATCTCCGAG (SEQ ID NO: 950) |
| 40 | CACTG | 61.508 | 451 | 290 | 161 | ATGTTCCAGGCGCCACGTAGAAAAGTAAA |
| | CATTC | | | | | AACAAAAAACAAAAAAGGTGAACTTAATTT |
| | CAGCC | | | | | TAATACTGTATTTTATTTAAACCAACCTATC |
| | TGGGA | | | | | CAAAATCTATCATTCTAGCATATAACCAAC |
| | G (SEQ | | | | | ATAAAAACTATCCTTGAGATATTTTACATA |
| | ID NO: | | | | | CTATAATATACTATGTAGTATATATAATATA |
| | 860) | | | | | CTATATACTATTTTACATACTACAGTATACT |
| | | | | | | GTAGTTGTTCATAGTATATCTTCAAAATCT |
| | | | | | | TGTTCTTTACATTTACAGACGATCTTAATC |
| | | | | | | AGACACTAAAGTTTCAAGGTCTAAGGTAA |
| | | | | | | ATATAGTCGTAGCAAATCAATAAAGTAAAC |
| | | | | | | TTGATATGAAATTATTTTTTACTGTATCAGT |
| | | | | | | TTGG |
| | | | | | | TTTTTTTGTTGTGGTGGTTTGTTTGTTTGTT |
| | | | | | | TGTTTGTTTGTTTGAGACAGAGTCTTGCTC |
| | | | | | | TGTCTCCCAGGCTGGAATGCAGTG (SEQ |
| | | | | | | ID NO: 951) |
| 41 | GCTTT | 61.508 | 356 | 137 | 219 | CAACACAATTCAGCCATGTTCTTTGTCCCT |
| | GGGAG | | | | | TTATAATAAGGATTGCCTTTCCTCAGGTTT |
| | GCCAT | | | | | CTCATAACATGTTCATTTCCATCTGAGGC |
| | GGCAG | | | | | CTCAACAGAAGCACCTTTAATGTTATTATC |
| | A (SEQ | | | | | TTTACCAACAGTCTCCTCAAGACAATGGA |
| | ID NO: | | | | | GATTTTTTTTCTATAAAGTGCCTCAAAACC |
| | 861) | | | | | CTTCCAGCCTTTATCCATAATTCAAATCCA |
| | | | | | | AAACCACATCCACATTTTTAGGTATTTGTT |
| | | | | | | ATAGGATCTGCCATTTAAAAATATTATTATT |
| | | | | | | ATTTTTGTATAGACAAGTTCTTACTATGCT |
| | | | | | | TCCTAGGCTGGTCTCAAACTTCTGGTCTC |
| | | | | | | AAGCAATTCTGCCATGGCCTCCCAAAGC |
| | | | | | | (SEQ ID NO: 952) |
| 42 | GAATG | 59.258 | 452 | 145 | 307 | TAGGGGGCACCTGTGCCAGTTTGTTACCT |
| | CCCAC | | | | | GGGTACATTGCATCATGCTGAAGTTTTTG |
| | TCTCA | | | | | AGATACAATAGATCCTATCATCCAGGTTG |
| | CTAGT | | | | | TGAGTATAGTGCTCAGTAGTTTTCCACCC |
| | CC | | | | | CTTGACACCCTCTTTCCCTCTTTCTCCTAG |
| | (SEQ ID | | | | | TAGTCTGCAGTGTCTATTTTTGCCATCTTT |
| | NO: 862) | | | | | GTGTTCATGAGTAACCCCAATGTTTAAAA |
| | | | | | | CCTACTTATAAGTGAGAAGATGCAGTATTT |
| | | | | | | GGTTTTCTGTTCCTGCATTAATTTGCTTAG |
| | | | | | | GATAATGGCTCCAGTCCTGGGAGCATTTT |
| | | | | | | GGCGGAGTCTTTAGGGTTTATTAGGTATA |
| | | | | | | GTATTATATCATCCACAAAGATGGTTTGAA |
| | | | | | | CTCTTTTCCTATTTGAATGTTTTTTATTTCT |
| | | | | | | TGTTATCTGATGGTTGTAGCTAGCAGTTC |
| | | | | | | CATCACTATGTTGAATAGGACTAGTGAGA |
| | | | | | | GTGGGCATTC (SEQ ID NO: 953) |
| 43 | GCTCA | 61.508 | 395 | 130 | 265 | GGCCATCCCTCTACTCCTGGTCTTTAAAG |
| | GGGGC | | | | | AATACACCACACAACATTGATTTTGTTCTC |
| | TGCCA | | | | | ATATTAATCACTCAGCCTTCACAGGAACA |
| | TGAGT | | | | | CTACTGGATGTGATCTCTCAAAAAGACTC |
| | A (SEQ | | | | | AGCCCTAGCTATCAACTCAAGTGCTGTCA |
| | ID NO: | | | | | GTAGTATCCATTGGACCCATAGAAAAATA |
| | 863) | | | | | TAGACATGCTGTTCCAGCAAGTCTCTGGA |
| | | | | | | AATGTATGAAACTCCTTTTGCTGGTCTCTC |
| | | | | | | TGCTTTCCTCGCTGTTTTGGGCTGTTTCA |
| | | | | | | ACCATGCTTGGCCCTTCATGTTTTGCAAA |
| | | | | | | CGATAACAATAACTAGGCATCAATCTCTTT |
| | | | | | | ACTTACCAAATCTAGGACACATAGGTCAA |
| | | | | | | ATTCTCCCTATATGCTCTCACTCTACTCAT |
| | | | | | | GGCAGCCCCTGAGC (SEQ ID NO: 954) |
| 44 | CTTCT | 60.298 | 428 | 160 | 268 | GAAGATCAGTCACTTGGCCTCTGGAGATT |
| | GCACA | | | | | CAGAACATAGGTTAAGCAATTTGAGACGC |
| | GTCAA | | | | | ATAGGAAGAGAACTTGGAGAATACCAATA |
| | GCTTT | | | | | TTTTACAGAATAGGCATAGAGAGGGAAAA |
| | TAACC | | | | | ATCATTAAGGAAACTAATAAAAACTACTCT |
| | ACG | | | | | ACAAGGTAAGAAACAAATTATAGAAAAAG |
| | (SEQ ID | | | | | AATGTAGTATCATAGAAGGCATTAGAAAA |
| | NO: 864) | | | | | GAGCTTCAAGAAGGAAGCACTGGTTAGC |
| | | | | | | AGTACCAGATATCCAAGAAATCTAGTAAG |
| | | | | | | ATGACAACTGAGAAGCCAATTGCATTTGG |
| | | | | | | CTATGTGGAGGTTACTGACATTTATGAAG |
| | | | | | | GCAGTTAAGTTGAGTGGGAAGGGCAGAT |
| | | | | | | TGTAGTAGGCTGAGGAGAATTAGGGAAT |
| | | | | | | GAAGAAGTAAAGACAATATAAACCACGTG |
| | | | | | | GTTAAAAGCTTGACTGTGCAGAAG (SEQ |
| | | | | | | ID NO: 955) |
| 45 | GGCAG | 61.508 | 368 | 127 | 241 | GGGCAGAATGGTCACCCAGCACTTGAAG |
| | ACTAG | | | | | ATCTCATCACACAGATAGCAAAGACCCCA |
| | GGCTG | | | | | TCTGGATGAGTTGATAGATAACAGAGGAG |
| | CACCT | | | | | CTGTCTTTGACCTTTGTCTCAAAACAACTC |
| | T (SEQ | | | | | TGTATTTATTTCCTAGGTAAAAACATAAGT |
| | ID NO: | | | | | GTCGCCCTCCTGTTCTCACCCATTTCTAC |
| | 865) | | | | | TTTAGGCTAGACAGAAAGTT |
| | | | | | | CTTACCTTCTTTGCTGGGTTTTTTATTTAC |
| | | | | | | CATGAGACACAACAAGGCAGAAATCTCTT |
| | | | | | | TAACATGCAGGCGTGATCCCCATCCTGTC |
| | | | | | | TTCAGAGTTGGCTGAGGGATCCCAGGCT |
| | | | | | | GATCTGATTTCTTTCAGACCTACAGAGCC |
| | | | | | | CATAAAGAAGGTGCAGCCCTAGTCTGCC |
| | | | | | | (SEQ ID NO: 956) |
| 46 | GCCTT | 61.508 | 399 | 132 | 267 | GTGCATTCAGGAAGCTTCTGTTTGACCAT |
| | GGCCT | | | | | TCATGTGTTTACAGATGTTAAAGGCATGG |
| | CCCAA | | | | | GTAATTAAAGTTAGCAATACCAAAATTTCT |
| | AGTGC | | | | | GGATTTGCACAAGTTATGAAAATGTCATTA |
| | T (SEQ | | | | | AATTCTTGATAAGGGTGTTGGTTGCAGAG |
| | ID NO: | | | | | TGTTTCTCAGAAAATATCTGTCTAGGAGA |
| | 866) | | | | | GAAGGAATGAAAAATTTTTAATAATTTTATT |
| | | | | | | TCAAAATAAAATATACATAAAGTTCCCAGA |
| | | | | | | GCAGCTGGGCTCAAATATTAATTCCTTCC |
| | | | | | | TTCCTACCAAGTTGCCTCCATTCTATATCT |
| | | | | | | ATCATTAATAGACAGCTGACCTAATAATTT |
| | | | | | | TAGTGGTTTCAAATTATATAGGCCGGGTG |
| | | | | | | TGGTGGCTCACGCCTTTAATCCCAGCACT |
| | | | | | | TTGGGAGGCCAAGGC (SEQ ID NO: 957) |
| 47 | CCATT | 60.908 | 406 | 152 | 254 | GCAGGAAGTGCCAGGACATTGAACAGAG |
| | AGGTA | | | | | CTCTCAAGTGAAGCTATTGTAAAATTTCAC |
| | TGCTT | | | | | TTCCTTCCTCCTTTGGCTTTCTAACTCCCA |
| | CCCAG | | | | | TGCTCTCCCAGAATGAGAGCTCTGGAAG |
| | GGAG | | | | | ATTTTGACTTCTTCCTTCTCTCTTTCCCTTC |
| | (SEQ ID | | | | | ACCCCTACATCAAGATTTTGTTTCAGAAAA |
| | NO: | | | | | TCTAATACTTTCATCTCTAGACTATTATCAA |
| | 867) | | | | | AAATTCCATGTCAGTATAGAGCTACTAACA |
| | | | | | | CATGTTACCCATTTCCAGACATTTAGATTA |
| | | | | | | ACTCTATATAATAAACTAAGTTTTTTGATCA |
| | | | | | | GCACCTTCTCAATTCTTCTGTTATGAAGTT |
| | | | | | | TTGGTTCTGGTGTTTTGTTTGTTTGTTTAG |
| | | | | | | GTTGGTGTCCTTTCTTCATTATTCTCCCTG |
| | | | | | | GGAAGCATACCTAATGG (SEQ ID NO: 958) |
| 48 | CAAGC | 60.908 | 418 | 169 | 249 | GCTGGAGGTGATGGCCAAGGTTCTGTTC |
| | TCCCT | | | | | TTCACTCTCTTAGAAGTGATGGTGTCACT |
| | GGTGA | | | | | CTGGGTAAGTCCCTCACCCTCTGAGCCT |
| | TTCAC | | | | | CATTTGTAAAATGTGAAAATCAAGAGAGG |
| | ATCC | | | | | CCCTGGTCCTTAAGGATCTTAAGGACTTT |
| | (SEQ ID | | | | | ACTAATGCCTTTTCCTCCTCTGGTCACAA |
| | NO: 868) | | | | | CATTGCAGAGACTTGTGCTGTCAAGGATA |
| | | | | | | CTGACTAGTTAAGATTCTTACTGCTGAAG |
| | | | | | | CTGCTTTGGGGAAGGACACATTTCCTGG |
| | | | | | | GAGAGCATGTCAAGCTGAAGGTCATCATA |
| | | | | | | CCAGGAAAAACAGGAGAAATTATCATATC |
| | | | | | | AGCAATAGGAAGGCAAAGTAAGGGTGTA |
| | | | | | | GGGTGTAGCCTAGATATTGCTGCCAAAAA |
| | | | | | | AGACAGGCCTCCAGTGCCTGTGGATGTG |
| | | | | | | AATCACCAGGGAGCTTG (SEQ ID NO: 959) |
| 49 | CCCAG | 60.908 | 427 | 156 | 271 | CCCAGTACTCCCACACTCTGCCTTCTTGC |
| | CTTGA | | | | | AAAGTAGATGGATCACTTCAATCCATCCT |
| | CATTT | | | | | CACTTCCTGGACCTCGCTACCTCCCACTT |
| | CCCTT | | | | | AGACTACTGATTGCAACAGTCTCTTCATTC |
| | TGGC | | | | | ACTCTTCCCATCCTACACTCTCGACCCCT |
| | (SEQ ID | | | | | CAAATCTATTCTCCACCCTGCAGAGTGTT |
| | NO: 869) | | | | | TTTCCTAACTGTATTAGTCTCCTATTTAAAA |
| | | | | | | TCCTTCTGTGGCTTCCACTTCAACCATCA |
| | | | | | | GGCTCTTCTAAGTTCATTTTGATAGCTATG |
| | | | | | | GGAAGCCTACTTTGACTTCATTCTCATTTC |
| | | | | | | TTCTCTAGGTTCCCTTCTGGTAGCTTCCC |
| | | | | | | ACCCTGAGCCTACATTCACCATGTCTGTT |
| | | | | | | GTATAGACTTTTTGAAAGGCAAATGAATCT |
| | | | | | | CAGGACCCCAAAATCACTAAGCCAAAGG |
| | | | | | | GAAATGTCAAGCTGGG (SEQ ID NO: 960) |
| 50 | GGAGG | 59.148 | 544 | 225 | 319 | GTCCAGGACAAAGCTCAGTAGCCTTTCGT |
| | AGGTT | | | | | AAGCAGCAGCCAACAATGCAAGAAAAGC |
| | GGTAA | | | | | AAGTGAATGAGGTAATGGATTTCTTTTCC |
| | AACTC | | | | | CTTATTCCACT |
| | CAC | | | | | AGAGAGCAGTGTTCTTTTGTACAAATCAG |
| | (SEQ ID | | | | | GTAGGTTGTTTGCTGCAAAGAACCTGTAG |
| | NO: | | | | | AGAAAATCTACATTAGGAGAGGTTTTATTA |
| | 870) | | | | | GCAGGAGAAAGGTCTCTTGCATAAAACTC |
| | | | | | | TGCATTCTGAACAAAAGGTGAAGACAGTT |
| | | | | | | TTTTCCAGCCAACTTGCTCTTATTGATGTA |
| | | | | | | GGTATACCTCTGATACCTTATTTTCATATG |
| | | | | | | GAAAAGTGTTGGTTATTCAAAAATATAACA |
| | | | | | | TGAAAACTATAGATTACATTCTATAGTAAA |
| | | | | | | TTTTTCAGATGGCAATCCTTTGCCAAAACA |
| | | | | | | GATCTGTCAGTAACCAACTATTAGTTATCT |
| | | | | | | TTATGGGTAATACAAAAATATTAATAATTTT |
| | | | | | | TTTAACAAAAACTCCTAAACAATTCAGGAG |
| | | | | | | CAAAAGAAAGAGTGAAGAGAAGGAATTTA |
| | | | | | | TGTGTGTTGTGGAGTTTTACCAACCTCCT |
| | | | | | | CC (SEQ ID NO: 961) |
| SIRT6-DdCBE | | | | | | |
| 1 | CTGTT | 61.288 | 372 | 126 | 246 | CAGCTGGCCAAGCAGGGATCACGTCCCT |
| | GCCTC | | | | | CTGCCTTGGCCACTCCAGGCCACCTGCA |
| | CTTGG | | | | | GAGGGGGCAGCCCTGTCAGTGATGGAG |
| | GTACA | | | | | GGGGAGACAGCCTGTGTTATTCACGCCG |
| | GC | | | | | GAAGTCCATGGCATTCTCAGAGAGCTCTT |
| | (SEQ ID | | | | | CCCCGTACCTCCCCAGAACACTGAGAAT |
| | NO: 871) | | | | | GGATGAATCAGTCCCGGCTCATCAGAGC |
| | | | | | | CTTTCATGCTAAACCAGCCCAAATGACTG |
| | | | | | | ATTTAGAAACTGCGTGGTCTTGTTCTTTGA |
| | | | | | | GTGGATTTCTGAGGCACACTCCAGAGCA |
| | | | | | | GGCCCAGGTCTTCATTATCTCCATGCACA |
| | | | | | | CCCTTTGATTAAGCATCTGTGACCACACA |
| | | | | | | GCACTGTGTGCTGTACCCAAGGAGGCAA |
| | | | | | | CAG (SEQ ID NO: 962) |
| 2 | CTTGG | 61.508 | 327 | 125 | 202 | GGGGGAGTCGGACTTAGAAGGTTGCCTC |
| | CACAC | | | | | TGCTGCTCTCCCACTAAGAACAATGCAGC |
| | AGCAG | | | | | TGCCCAGGAAACACACCCGAGCTATGAA |
| | GTGCT | | | | | TAGGCTCAAGCACACAGGGCGAAGCTGT |
| | C (SEQ | | | | | CGCATTTCTGCCGTTTAGAAAGGATCTCT |
| | ID NO: | | | | | AGTGTGAAGTTGCTTGGAGGCTCTGATTT |
| | 872) | | | | | TGCAAACCCAATTAAATATAGATTTCTTTG |
| | | | | | | GGGTGACGGTTGGGAAGGGTGTGGGTG |
| | | | | | | GAATGTATGTCTGTGCTGCAATGCAATTT |
| | | | | | | AATCCTGAAAGTTTCAGCAAACCTATTCAC |
| | | | | | | ACACTCCATAAACATTTCTGAGCACCTGC |
| | | | | | | TGTGTGCCAAG (SEQ ID NO: 311) |
| 3 | CCCCA | 61.508 | 411 | 259 | 152 | TGCTAGGCCCCGATTTGTGGACACTGAG |
| | CTTCC | | | | | CTAGGCAAAAGGATATATAAAGAGATGCA |
| | TTCCT | | | | | AGCTAGAGGAGGGGAAAGTTCTGGATGG |
| | CACCC | | | | | GGACCTCATTGGCCTCCTCACCTCTGTAG |
| | T (SEQ | | | | | ACCCACGTCTTGGGTTGACCCAGGGTAG |
| | ID NO: | | | | | AACCCTATAAGGATTTGTTCAATAAACGAA |
| | 873) | | | | | TGCATAGAGACTGATCAACAACTGACAAA |
| | | | | | | GTGGTATTTTACAGATGCCGTGAAAGCAG |
| | | | | | | CATACAAGCCGGAGAGCCTACTGGAAGC |
| | | | | | | AAGAGGGGAAAACCCAGCCGGGTATGG |
| | | | | | | GGGCTGCGGGGAGGATGCCAGGTGGCG |
| | | | | | | GAGACTGGATTCAGGATTTGATGCTTGAA |
| | | | | | | CTGAACCTTCTTTTAAGAGGTGAGTGGGC |
| | | | | | | TGGGAGGTTGTGATGCCCAGAGGGTGAG |
| | | | | | | GAAGGAAGTGGGG (SEQ ID NO: 312) |
| 4 | GCTCT | 61.508 | 334 | 205 | 129 | GACCCTTCCATTAGATCTGAGGGGGGGG |
| | GGCTG | | | | | TGGGAGGGCAGAGGAGGGGAGGACAGA |
| | GAGCA | | | | | GAAGTGGGAGGAAGCAGGAGAGGGGGT |
| | CAGGA | | | | | GGAGGGGTAGGGGCACAGTGTCATGCCT |
| | A (SEQ | | | | | CTCGGGCTTCCCCCACAGGGTGAATGAC |
| | ID NO: | | | | | TGTGTGCTGCGGGTGAATGAGGTGGACG |
| | 874) | | | | | TGTCGGAGGTGGTACACAGCCGGGGGG |
| | | | | | | TGGAGGCGCTGAAGGAGGCAGGCCCTG |
| | | | | | | TGGTGCGATTGGTGGTGCGGAGGCG |
| | | | | | | ACAGCCTCCACCCGAGACCATCATGGAG |
| | | | | | | GTCAACCTGCTCAAAGGGCCCAAAGGTG |
| | | | | | | CGGCCCTCCAGGTTCCTGTGCTCCAGCC |
| | | | | | | AGAGC (SEQ ID NO: 313) |
| 5 | CCTCA | 61.508 | 379 | 229 | 150 | GTGTACCCCAGAGAGAGCCGAGCCTCTC |
| | GCCTC | | | | | TTTGCAGAGGCGGTTTCCCTTCTGTCTGC |
| | CACAT | | | | | CTCGATCACTGCACTGGCTGGGAGGGCT |
| | CTGCC | | | | | TTGCGTGGTTTACACCATGCAACACGTTT |
| | T (SEQ | | | | | TCCATCTCAGCATCTTATGCGCTTAAGCA |
| | ID NO: | | | | | GCGGTTGGGCGTTAGCCCTGCTCAGGCC |
| | 875) | | | | | TCCAATGCTAACAAAAACAGGGAGAGCCT |
| | | | | | | CCATTCACCCCCCAGGGCCTCGTGTGCA |
| | | | | | | GGGACAGGCAGCCCCGACGCACTGCTG |
| | | | | | | TCGGCCACCTCCTGGGTTAGAAACTGCA |
| | | | | | | AGGCCTGATGTGGGGCCTTCCCAGCTTC |
| | | | | | | CAGGGGGCTATGTCCTTATCCTGAGACC |
| | | | | | | CCACAGCCAGGACCGCAACAGGCAGATG |
| | | | | | | TGGAGGCTGAGG (SEQ ID NO: 963) |
| 6 | GTCCA | 61.288 | 350 | 126 | 224 | CCTCAGGTGAGAGCACAGCCTTGGGGAC |
| | AGGTC | | | | | ACAGCCTGGGGCCTTGTGGCATGCCCCA |
| | ACAGA | | | | | GCAGGTGCTGGGGTCGGGGGTGAGCTG |
| | GCTGG | | | | | GAATCTGGGGTAGGTACCTCAGCCGGGG |
| | AG | | | | | TGTCATAGTCCCTGAAAGGAGGGAGGCT |
| | (SEQ ID | | | | | CTCCTGTGTGATGGGGAGATGAAAGCCT |
| | NO: | | | | | AGGCGACGGGGTTTGAGGGATTTCAGGG |
| | 876) | | | | | ACCGAGACAAAGGGGATGAGGGAGGGG |
| | | | | | | AGGAGACCTGCAACAATCTAGAACTACCT |
| | | | | | | CCTGCCCTTCCCCCATTGTGGAGATTACA |
| | | | | | | CAGACTCCAGAGTCAGCACAGCTGGGTA |
| | | | | | | CAAATTCTTAACACTCTGCTCTCCAGCTCT |
| | | | | | | GTGACCTTGGAC (SEQ ID NO: 964) |
| 7 | ACCAC | 61.508 | 333 | 208 | 125 | GGATCCAAGGTGCCTTCACCCTGCGGCC |
| | AGAGC | | | | | TGCTCTATGAGGGCCTGCGTCGCACATC |
| | CTCAC | | | | | AGCTGTTGGTATCCTCACCTCGCTGAGCC |
| | CCACC | | | | | GTTCCTCCGAGTGTGCTCTGTGGCTTGCA |
| | A (SEQ | | | | | CCAGGCCTAGCTGGGCCTTGTGTCTGGT |
| | ID NO: | | | | | CTCATCCAGCGTCTTCTGATTGTGGGGAA |
| | 877) | | | | | TGAGGCAGAGGTCTCCCCACCTGGGCTG |
| | | | | | | TCCGTGTGGCGAAAGCCTTCCGGGGGGA |
| | | | | | | GGGAGAGGAAGTGCCTCTACAAGGGCCT |
| | | | | | | AGAGTCGTCCTGTGGCACTGAGGGCTCT |
| | | | | | | GGCAGCTGCACACAGGTTGATAATGTCA |
| | | | | | | CTGGTGGGTGAGGCTCTGTGGT (SEQ ID |
| | | | | | | NO: 965) |
| 8 | CAGGC | 61.508 | 403 | 251 | 152 | GCTGGATTCGATCTGAGGTCAGCTTGACT |
| | CCTAC | | | | | TTTCCTTCAGGATGATGCGCTGCTTCCTC |
| | TGGGA | | | | | TGATGAAGGATCAGGGATGCAGAATGGG |
| | CGAAC | | | | | ACTGATGTATCTGTCCAATCATCCCACTC |
| | A (SEQ | | | | | ATTCTCCTCACTTTGCATGCTCTTCTCGG |
| | ID NO: | | | | | GGCCATCTACAGACAGGTGAGGGGCCCC |
| | 878) | | | | | AGGCAGACAGCTCCTTGGGACACTGAGC |
| | | | | | | TCCACATTGAAATGTTACTCAAATGAATGC |
| | | | | | | GCAGAGGAGCTGTGAGTGAGAAAGTAAG |
| | | | | | | CCCTCCAGCGTGAAGAGCCCCGGAAAAG |
| | | | | | | CCACCCTTGCTCTGGCACCTGTCATGCA |
| | | | | | | GCCCTCACAGCAGTACTCCCATGGGGTA |
| | | | | | | TGGGAGGACAGGGGCACTTCTGTTGGTC |
| | | | | | | TTTCCTTCCTCATGTTCGTCCCAGTAGGG |
| | | | | | | CCTG (SEQ ID NO: 315) |
| 9 | CTCTT | 61.088 | 343 | 215 | 128 | CCCAGGACAAAGTTGCTTTGGGGGGCTT |
| | GCACA | | | | | ACAGGTTGAATCACCCATAGCTATGTTTT |
| | AGGTT | | | | | GTTTGGCTTGTACTGTGTTGTTTACATTAT |
| | GCCAC | | | | | TTTATTAGTTGGCAATAGCTTTAAATCAAA |
| | CTC | | | | | ATTTACATTAAGGTCCAGATTTGGGGCTC |
| | (SEQ ID | | | | | CTCTTAAAAAAATAAATCAGCACATCTACA |
| | NO: 879) | | | | | ACAGTAGGATTTTCACGCCCAATGGCAAA |
| | | | | | | ATCTGTTAAAGCTGTGTGACAGCTTCTCC |
| | | | | | | TTTTAATGGAGATGACCTCTTCAGACCCT |
| | | | | | | GTACTCCAGGTGCCACAGGAACCAGCTG |
| | | | | | | CC |
| | | | | | | CGGTTCATCCTTGTGTGTTACCTGCATGA |
| | | | | | | GGTGGCAACCTTGTGCAAGAG (SEQ ID |
| | | | | | | NO: 316) |
| 10 | ATTCC | 61.508 | 473 | 280 | 193 | GCCTCGCCAAGAAACGCCCATTTATTTCC |
| | GGGTA | | | | | TTCCTATTTATTTATTTTGCAAAAGCCGCC |
| | GGCGA | | | | | TTTTGAAAGCCGGAGTGCAGTGCGGACT |
| | GGAGG | | | | | GGCTGAGGCCGGGGCCCAGGCGCGCG |
| | T (SEQ | | | | | CCTCCTTCCTGCAGGGGGCCCGGGGCAT |
| | ID NO: | | | | | CGATCGGGGGGGGCGTAATGAACCCTAA |
| | 880) | | | | | TAACAGCTCTATCACCGCCCGCCCGCCA |
| | | | | | | ATTGGCCCGGGTGCCCTCCAAATTAGCC |
| | | | | | | ACAAAGAAGCCAGTCTGTCAATATTAATTC |
| | | | | | | CGCCGCGGAGATTACTTGTTGTGGGAGA |
| | | | | | | ACAAAGGTGTTCTGGCTTGAGCTGAACAA |
| | | | | | | CTTAACTCCTTGTCTCATAAAATTTAAGCT |
| | | | | | | GCTATTGATCGCCTCCTTGCATTGTGTGA |
| | | | | | | GGATTAAACACGTGCGCGTGCACATCCA |
| | | | | | | GGCACACGCGCGCACACACTCGGGGGG |
| | | | | | | CCCGGCCCCGCTCAGGCAGCCGCCCCA |
| | | | | | | CCTCCTCGCCTACCCGGAAT (SEQ ID NO: |
| | | | | | | 317) |
| 11 | AGGGC | 61.508 | 327 | 202 | 125 | CCAGCACACCCTGATAAGCAGGATTCAG |
| | CCCAG | | | | | ATTGGGCATGGGACAGGACAAAGGCTCT |
| | TGAGT | | | | | GAGGAGGCATGAGTGGAGTAGTAGAGAG |
| | GTGTG | | | | | AACACAGGACTCTGGGTTCTGAGCCAAG |
| | T (SEQ | | | | | CTCTGCCATCCTGCAGCCATCTGACCCCT |
| | ID NO: | | | | | GGCCAGGCCTTCCCCCAACTTCCTCAGC |
| | 881) | | | | | TGTGAAACGCGGCAGGGCTGCAGATGAC |
| | | | | | | GTAGGTGGGCAGAGGCCCTCTGGTGCCT |
| | | | | | | ACTGCTAGACACCCTTCTACTCTGGTGGG |
| | | | | | | AGACCTTGTGCTCAAACGCTCTCAGAAGT |
| | | | | | | CAGGGCAGTTGGGAGTCCAGGTCACACA |
| | | | | | | CACTCACTGGGGCCCT (SEQ ID NO: 318) |
| 12 | GCCCA | 61.508 | 358 | 141 | 217 | CCAGGGCAACAGGTTTAAGACCCATTAG |
| | CATCA | | | | | GAGACAATATTGTCGTCATGAATATAAATG |
| | GCTCT | | | | | AGGCCTAGGCAGGTTGGGAGTAGCTAGT |
| | GCACC | | | | | GTCTTGGCTCTGGCACAACCAGGCCCTG |
| | A (SEQ | | | | | TTCCTCCACAGGGCTGGCCAGGGCAGAG |
| | ID NO: | | | | | GTGAAAGGGGCTGGGGTGTGACCGAGT |
| | 882) | | | | | GAGCTGGGGTAGAGGTAGAAAAAGGGAC |
| | | | | | | ACAGAACATCCCAGGCCTAAGCTACATG |
| | | | | | | GAGTATGAGAGAACAGCTGGAATGTGCT |
| | | | | | | CTAGAAGCCCTGGGAGAAGGCCTCAGTT |
| | | | | | | GAGGCTGAATTCAGATATGCCTGGGACC |
| | | | | | | CGGTCTCTGTTAAGAGACCCCGGAGAGC |
| | | | | | | TGGTGCAGAGCTGATGTGGGC (SEQ ID |
| | | | | | | NO: 319) |
| 13 | CAGCC | 61.288 | 442 | 132 | 310 | GGTCTCGATGTGCTCCGCATGAGTGGCA |
| | TTCAC | | | | | TCCGTACCTGGAAGCCACTTGCTGGTTAC |
| | TGCAG | | | | | TCTGAGGTAATACAGACAACAGCTCCACC |
| | TT | | | | | CGGGTGGCGCATGGGCACCTTGCTCACC |
| | (SEQ ID | | | | | CCGGAATCCCTCCTTTGCCTGGACAGCC |
| | NO: 883) | | | | | TATGCAGGTAAGATGCCTTCATTTCATTCC |
| | GCTCC | | | | | CATATAGCTCATTCAAGAAATCACAATGG |
| | | | | | | CAACCTCAACTCTCTTGTTCCCTCACATC |
| | | | | | | CCATTACTCAGGAAAGCTGTCAATTCCAG |
| | | | | | | CTTCAAAGCTCTTTTTAAAAATTCTCTTCC |
| | | | | | | CCATGTGTCCAAGCCATACTTGCAGCTTG |
| | | | | | | TGTCCCTATTTCTAGTCTCTTCCTCTACCC |
| | | | | | | TAACCATTCTTTAAGCTGTGGCCAAAGGG |
| | | | | | | ACCTTTCAAAATGTAGCTCTGCCATGAAG |
| | | | | | | GAAATGCAAATTAAAACTGCAGTGAAGGC |
| | | | | | | TGGGAGC (SEQ ID NO: 966) |
| 14 | CTCTC | 61.508 | 408 | 159 | 249 | CCATCACCTGCCTCTCCCTGTCTATTGTG |
| | CTCCC | | | | | CTATTCAATGAAACATCATAGAGAGGATA |
| | CAGCA | | | | | GATGTGATTCAGCATCTGGCATATACCTG |
| | GGGTT | | | | | CTGTAGATGCTGTAGAACAGATTCTCTGC |
| | A (SEQ | | | | | AAGTGCTTGTTAATGTAAGTTCTCTCCGC |
| | ID NO: | | | | | AGAACCTCATTCTCTTGAGACAGCAATGC |
| | 884) | | | | | TGAGAAAGCAAATCTGGGACTGGGGCTG |
| | | | | | | TGATATATTCAAATTTGACAGCCTC |
| | | | | | | ATCCTCCTCCTAGGTGATTCTGATGTCAG |
| | | | | | | TGGTCCTGAGGCCATATTTTCAGGAATAC |
| | | | | | | TGCCTTAAAAGTCATTATTATCCAACCAAC |
| | | | | | | GAATTCTTACTCTGTAGGCAACTAAAGGT |
| | | | | | | TGACCCTTCCCAAGGGTCACATTTTAATG |
| | | | | | | GTGTGTTTCACTTTTAACCCTGCTGGGGA |
| | | | | | | GGAGAG (SEQ ID NO: 967) |
| 15 | GAAGG | 60.298 | 474 | 160 | 314 | CTTGGTGTGTCTCTTCCTGCACGTAAAAT |
| | ATGTG | | | | | TTCATAGAGAAATATAACAGCCTCCCTCAT |
| | GAAGC | | | | | CTTCCTGTCTGATTAATTTAAGAATGCCAC |
| | ACTTG | | | | | AATTTTCTTGGAATAGCCTCTTGGTGATGT |
| | AAATT | | | | | CCTAGCCTCTTCTTATGAGGGTGAGTTGT |
| | CCC | | | | | CAGGAACTAGGATTTCACCGAGGCCTTA |
| | (SEQ ID | | | | | GGGTGTGAATGAGGAGGAGAGTCTGCTG |
| | NO: 885) | | | | | GAAATGTTTCTAATAAAGAAGGTAAGCAA |
| | | | | | | AGGTCAGTGACTTCTCTGTAGTCACCAAC |
| | | | | | | ATGACCTGTAGCCTACTTATTAATGTGGTT |
| | | | | | | CACAATATTTAAGAAATGAGTCATATATGA |
| | | | | | | TGAGGAAACCCTTGTTCTTCAAGAGTTAC |
| | | | | | | CAAAGTGGTCTGGTTTTCCACTATTTTCTA |
| | | | | | | TTGCCTTGTGTTAAGAGTTTAAGATAGCAT |
| | | | | | | AATACTGTCAGAGATGTATTTGCATGTATC |
| | | | | | | TTTTAGGGAATTTCAAGTGCTTCCACATCC |
| | | | | | | TTC (SEQ ID NO: 968) |
| 16 | TGGGC | 61.508 | 363 | 142 | 221 | GGTAAAAGCACCCTGAGAGGGCATGGAA |
| | CTGGG | | | | | AAACACCAGGTGAGTTGGCCCCTAAGCT |
| | ACTAA | | | | | ACCAGGGGCATTAGTTCCAGACATCTCCT |
| | CCAGT | | | | | GCTGAACATGGAGGCAGGTGGGGTCCCT |
| | G (SEQ | | | | | GATTTCACCCAGCCTGGCTGTGATGCACT |
| | ID NO: | | | | | TCTGAGGGAGTCCTGTGTGAAGTGCACA |
| | 886) | | | | | GGGAAACCCTGCTCTCTCAGGAAGAGTG |
| | | | | | | AGGTATGGTATGGTCTGAATGAGGGGAG |
| | | | | | | CAGGGAAAGCTGCCAGACTCAGGCTCCC |
| | | | | | | AAGGAAAAGTTAGGACCCAGTGGGGAGT |
| | | | | | | AGGGACCTTGAGAAATGAGACTGGTCAG |
| | | | | | | AGGCTATGTTCACATCCCAGGCAATGTGG |
| | | | | | | GGCCACTGGTTAGTCCCAGGCCCA (SEQ |
| | | | | | | ID NO: 969) |
| 17 | GCCCT | 59.508 | 370 | 141 | 229 | CAGTCTCCACAAGTGACGGGAAATTGGA |
| | GCATC | | | | | ACCATGCACAAGACACCAAAAGCAGTGG |
| | TTAGC | | | | | ACAGTCCACCAGCAAAAAGGTAGTGCTCA |
| | CCTTG | | | | | GCAATGTGGCTTTCTCTACACAGGGGATG |
| | (SEQ ID | | | | | GCTCAGCGACGTGGCTGTCTTTACATAG |
| | NO: | | | | | GGGGTGGCTCAGCGGCGTGACTGTCTCT |
| | 887) | | | | | ACACAGGGGATGGCTCAGCGATGTAACT |
| | | | | | | GTCTACACAGGGGGGGGCGTTTCCTTC |
| | | | | | | TCGGAGTATTAGCGCTTAGTTGACTGGAG |
| | | | | | | TATTAGAGCAGTGTGGTTCACAAATAACT |
| | | | | | | CAAAATTGCAGTTAAAATAAAGTGAACTAA |
| | | | | | | AATATTCAAAAATAGATTTAGTGTAAAGTT |
| | | | | | | TATATGCAAGGGCTAAGATGCAGGGC |
| | | | | | | (SEQ ID NO: 970) |
| 18 | GCCCC | 61.508 | 341 | 125 | 216 | AGGGCAGCAGGTGAATGCGCATCTCAGA |
| | AACAT | | | | | GAGCAGCCGGGTCACCCGTGGGTTCCCT |
| | CACCC | | | | | CGCAGGAACTCATGGCACAGGAACTGCA |
| | GCATC | | | | | TCAGGAGCAGAAGCAACTCCCGCCCCAG |
| | T (SEQ | | | | | GGCCTCGTTCCCATGCATGCCAGCCACG |
| | ID NO: | | | | | TAGCGCACCTCAGGCTCCCCTGGGGACA |
| | 888) | | | | | CATGGGGGCTTGCAGCGGGTTCATGCCT |
| | | | | | | GGGGCCCTGCCCTGTGCCTACCTCTCCC |
| | | | | | | CACTCCCCATGCCAGTACCCAGCTCATG |
| | | | | | | CTCCCCAGGCTTGTCCGACATTTCCATCA |
| | | | | | | CATACAGCTTCAGGCCCTGGTAGCTCTTC |
| | | | | | | CCAATGCTGTAGATGGGGGTGATGTTGG |
| | | | | | | GGC (SEQ ID NO: 971) |
| 19 | CCAAA | 61.088 | 345 | 217 | 128 | GGCCCTGGCTTGGCCTTAACTCTGCTCC |
| | CTAGG | | | | | AAAATCAGACTGGGTGCTGGGATCAGGG |
| | CCTTA | | | | | AAAGGGGGGCAGAGAGAGTAGGCATTT |
| | GGGCC | | | | | CTGAGCCTGCAGGGGAAGGGGTGCTTCC |
| | TAC | | | | | CAGGCCACGAGAGCATAGGAATGCCTAG |
| | (SEQ ID | | | | | ATCTGCAGCCATGGCCTAGGAGGGCAGA |
| | NO: 889) | | | | | GGTCCAGCTCCTCCAACTCA |
| | | | | | | GAAGGGGGCGGGGCTTCCACCTG |
| | | | | | | TCCCAGTCTCCTGCAGGCTCTGTG |
| | | | | | | GCATGAAAAGCCCCACTGTGTCTC |
| | | | | | | CCCCACTGCAGCTGGTGTCTTCAC |
| | | | | | | AGCAGTGGCTCTAGACAGGTCGC |
| | | | | | | TGCCATCAATAAATAAGTAGGCCC |
| | | | | | | TAAGGCCTAGTTTGG (SEQ ID NO: |
| | | | | | | 972) |
| 20 | AGAGC | 61.088 | 325 | 200 | 125 | GCCACTCCCCTTTGGTGCTGTGAC |
| | TGTGT | | | | | CTGCAGACCCATTCATCATGGACT |
| | GGCGT | | | | | TTTCCTCCTGCCTCAGAGCCCTTC |
| | GAACG | | | | | TCTTCATTCTACTCCAGCCCTCCA |
| | CTA | | | | | CCTTGGGGACCTAATTACCCACAT |
| | (SEQ ID | | | | | GAATGACCCGTCCGGTACCCCTG |
| | NO: 890) | | | | | CCTCTCACCACTTGACTTCCTCTC |
| | | | | | | CTTCCCTTCCCACCAGCTGGCCTC |
| | | | | | | TCCGTGTTCCCGTGGGACAGTCTC |
| | | | | | | GCCACCCAAAATTCCACCACACCC |
| | | | | | | CAGTACTTGGTTTCAAGCCACGCA |
| | | | | | | CCCTGACCACCATTCCTTATCCTT |
| | | | | | | CCAGCTTACTGGCTTTAGCGTTCA |
| | | | | | | CGCCACACAGCTCT (SEQ ID NO: |
| | | | | | | 973) |
| 21 | CAGGT | 62.768 | 351 | 138 | 213 | CCAGGAGAAGAAGTGGCATCCGC |
| | GCTGT | | | | | GGGGCAGGGCGGGCGGTGACGG |
| | TGAAG | | | | | CCGGCTCAGGTGGCGGCCCCACC |
| | GCGGC | | | | | CCAGCGCCCTTCAGCAGGCCTGG |
| | CAAA | | | | | GTCGGTCGGCCTCGGACCCCGTA |
| | (SEQ ID | | | | | GGCGCCGGCGGAGCTGGCGGCA |
| | NO: 891) | | | | | GGGACCCGACAGCTCTGCCGCGG |
| | | | | | | ACTCCGCAGGGGACATCCCCCAC |
| | | | | | | CCTGGCGGCCCACCCAGGCGGAG |
| | | | | | | CGCGGCGGGGACCGTGCATCTAG |
| | | | | | | CGCGCCCTGGTGACCTGCGGGCC |
| | | | | | | GAGGCGGGCTGTGGGGCTTGGCC |
| | | | | | | CCGCGACTCAGCCTTCCGGGCCC |
| | | | | | | ACAGCGGCCCTGACCCGGGAAGT |
| | | | | | | GCGGAGGTTTGGCCGCCTTCAAC |
| | | | | | | AGCACCTG (SEQ ID NO: 974) |
| 22 | CAACC | 61.508 | 338 | 131 | 207 | CCTTTGCAATGGAGGGCAGGGAGGGCAC |
| | TCCTG | | | | | CCTGCCCTCAGTCTGGCTCCTGATCTAAC |
| | CCTAC | | | | | ACCAGGAACCTCAAGTAGCCAAAATCCTT |
| | CTCCC | | | | | TAGGAGACAAGGTTCAGGTTTCACACAG |
| | A (SEQ | | | | | GCTGTCCTGTTTAGAGCAAATAAACAGCC |
| | ID NO: | | | | | CATTCGTGTATATTTTGGACCAGGTCTCT |
| | 892) | | | | | GGACTGCCCGGGGGCACCTTGCCACCT |
| | | | | | | GTTGTGCAACCTGCTTGTTGTGGAGAATT |
| | | | | | | AGGGAAGAACAATTCTAGAAAGGGGTCC |
| | | | | | | CAAGTCAGTTAGCTGAGACGGGGTATTG |
| | | | | | | AGAGTCCTTTTGGGATGCACTTCACATGG |
| | | | | | | ATTCTGGGAGGTAGGCAGGAGGTTG |
| | | | | | | (SEQ ID NO: 975) |
| 23 | CCACA | 60.058 | 434 | 263 | 171 | CAGGCCTAAGCCTTGATAGACAGAATTTT |
| | CAAAC | | | | | ATAAAATATTTTATATTTTCATTTGAAGAAA |
| | AAAAG | | | | | ATGTTATAAACACATAAGATTTGAAAACAG |
| | GATCT | | | | | TAAGCACAAAACACAACAATCTAAGGTGT |
| | CCTCT | | | | | TTTCAAGAGCCACATAAAAGGATTGAAAA |
| | TGAGT | | | | | TCAATTTGGCAATCTTTCTGAAGGGTAGG |
| | (SEQ ID | | | | | TATTTAGACAGAGTGCTTCCAGTGGCTGG |
| | NO: 893) | | | | | AAATCATTAGAAAGCCAAGGCAAGGTGCC |
| | | | | | | CATGGTCACGCAGAAGGGTCCCTGAAGG |
| | | | | | | AGCCACAGCTCTGCAGTGTATTCTCTTGG |
| | | | | | | ATGATGACAGAGGCCAATTATTTAATGATA |
| | | | | | | CAGGGACCGATGTGGTCATTCGATAAAGT |
| | | | | | | TCAGTGCTTTAGATATGATTGAGACAGAA |
| | | | | | | TGAGTGACTCTTAAGATTATTCATACTCAA |
| | | | | | | GAGGAGATCCTTTTGTTTGTGTGG (SEQ ID |
| | | | | | | NO: 976) |
| 24 | CCCTG | 61.508 | 340 | 128 | 212 | CAAGAGAGCTACTACCTCGGAGCAGCCT |
| | ACTCG | | | | | TGTCTCAGACCTGCAGCCACCGTGTCTC |
| | AGCGA | | | | | GACCCTCCCTCCCTGATCCACTCTGCCC |
| | TTCTC | | | | | GTCAGCAGGATTCCGGTCTACACCCCGC |
| | C (SEQ | | | | | AGAGACTCCGCAGCGTACGAGGAGAACC |
| | ID NO: | | | | | CCGCCTTCTACCAGAAAAAGAAGCGACTT |
| | 894) | | | | | CCTAGATCTCCCGGAAGTCCCTTTCGCTC |
| | | | | | | CCAGCGCTTCGCGTACCAGGAAGGGGAC |
| | | | | | | GGCGTCCTGAAAGGGACATTTGAAAAAC |
| | | | | | | GGAGGCAACGCGTCTATTAATTAGGTGG |
| | | | | | | CGG |
| | | | | | | CGCCTGGGGTCGCAGCACTCCAGAGGCT |
| | | | | | | GAGGTGGGAGAATCGCTCGAGTCAGGG |
| | | | | | | (SEQ ID NO: 977) |
| 25 | CGCAC | 61.508 | 487 | 201 | 286 | CTCACCTGCCCTGGGACTGAAGCGGGG |
| | GAATT | | | | | GCCACCCCGAGCCCGGGGGGGGGGGG |
| | AGGCC | | | | | GCGGCGGCGCAGGGGGAGGAGGGCAG |
| | CGAGC | | | | | CGCGGGGCTCCTGGCACAGCGGCCGCC |
| | T (SEQ | | | | | GGGCGCCTGGTGCTGCCGAGCCGCGGA |
| | ID NO: | | | | | CCCGCCCAGAGATATAAATAACATTATCT |
| | 895) | | | | | GAGGAGGGACATTCCTGCCGGGGGACAT |
| | | | | | | CGCTGCTGCCGGGACCAGATAGTCCCGC |
| | | | | | | GGGGGGGGGGGGGAGGGGGGGCCGCA |
| | | | | | | GCCTCCCGCAGCGCCAAGCCCAGCCCC |
| | | | | | | CGCCTTCCCCACCCACTCACGCACCTGC |
| | | | | | | CAATCAGCCTCCGCGCCTGGGGGCACCT |
| | | | | | | GCCGGGCCTCAGCCCGGCTCCTGGAAC |
| | | | | | | CCGCCTCTGCCTCCCCGCCCGCCGCTGA |
| | | | | | | GCCTGGGGCGCCTCCATCGTCGTTCATT |
| | | | | | | GCCCCTAACCCCGCGCCACCCCGAGCC |
| | | | | | | CCCTGAGGGGGGAAGGGGGTTGGACTC |
| | | | | | | TAGCTCGGGCCTAATTCGTGCG (SEQ ID |
| | | | | | | NO: 978) |
| 26 | GCTTG | 62.768 | 325 | 200 | 125 | ACAACTCCTGGGGGGTCCAGATGCTCAC |
| | AACCC | | | | | AGCTGGAGAAAGTGAACATTGACTGGGG |
| | AGATA | | | | | TGGCAGTGTGGGGCCGGGTGAAGGCAG |
| | GCCTG | | | | | CAACTGCGGAGGAGGCAAAGAAGCCCAG |
| | GCTC | | | | | GGAAGGGTGGGCAGGAGCTGCTGGAGT |
| | (SEQ ID | | | | | CAGAGAATGCCCTAGAGGCAAATCTGGG |
| | NO: 896) | | | | | GCCTGCCTCTTCCCGCCAGAGTACCAGA |
| | | | | | | GCCTGACTCAGATAGCCCAGCCCCAGAA |
| | | | | | | CCCCACAGAGGGACATGAAGGGAAGGCA |
| | | | | | | CAGCCATCCCTCTCTCCAGGGGGCCCCA |
| | | | | | | TGCTAGAGAGAGGCAGTGGTTAAGAGCC |
| | | | | | | AGGCTATCTGGGTTCAAGC (SEQ ID NO: |
| | | | | | | 979) |
| 27 | GGAAG | 61.508 | 360 | 135 | 225 | GCTGGCTGCTCATGGTCCATGTCTTCTCA |
| | GTGCT | | | | | GCTCCAGTATCCAACAGTACTAGCCCGTT |
| | GCCGC | | | | | TCTGCTGAGGTTGCTTACCTTTGGCAGGG |
| | TTCTCT | | | | | AGCCTCTTGGGCAGGAGTCCTGTTTCCCT |
| | (SEQ ID | | | | | CCTGAGGACCCACACCTGTCTGTTGCCT |
| | NO: 897) | | | | | GCCCTGCTGCCTACCATTGAAACAGGGC |
| | | | | | | TTGCTCCAACACAGCCACCTCTTTTATCTC |
| | | | | | | TGCCACCAGGCACCTCCAGCCTTGGGTC |
| | | | | | | CTTATTCCTTTCAGGTATGGGTCAAGTACT |
| | | | | | | AGTTCTTTTTTCTCCTCAGACTTCAGGGAA |
| | | | | | | CACATGTCAAGCACTCCAAGGGAACCTGT |
| | | | | | | GGGAAGCCCTTTCACAATGAAGAGAAGC |
| | | | | | | GGCAGCACCTTCC (SEQ ID NO: 980) |
| 28 | GAGTG | 61.508 | 408 | 158 | 250 | CCCTGCCTGCAATGGTGAGCTACATCTG |
| | AGGTG | | | | | CATCTTCCTATTTCCTTTCCCTGTCTTTGA |
| | CAACT | | | | | AATCCAGTAGAGAAGCCCAGACCGCACA |
| | GCTGC | | | | | ATGGGAACATCCCACCTCTCAGATGGCAA |
| | G (SEQ | | | | | TGCTGCCCAAGCCGCTCCACCCCCAGGG |
| | ID NO: | | | | | GCCTTGCTTGCATGTGCCCGAGGCCAGC |
| | 898) | | | | | CCTGCAGGGCAGACAGTAGCACAAGAGC |
| | | | | | | ACCATCTTGGTCACAAAGTTGTCTAAGCT |
| | | | | | | CAAAGACAGCCCCGGAAAAACACCTGAC |
| | | | | | | ACACTCCACAAATAATAATCAGCAGATCTT |
| | | | | | | CATGCCATCCCATGTGACAGATGGGGAG |
| | | | | | | CAGAGGCCTAGGGGGCTGCTGGCCCCTT |
| | | | | | | TGCTGAGAGACCCCTGACAAAGTTAGTCC |
| | | | | | | AATGCCTCTGCAAAATCGCAGCAGTTGCA |
| | | | | | | CCTCACTC (SEQ ID NO: 981) |
| 29 | GCTGG | 61.088 | 332 | 206 | 126 | CTGTGGGTGCAGCTTCAGCAGACTTAAAT |
| | AGTTT | | | | | GTTCTTGCCTGCCAGCTCTGAAGAGAGC |
| | GCTGA | | | | | AGCAGATCTCCCAGCACAGTGCTTGAGC |
| | AGGTC | | | | | ACTGCTAAGGGACAGACTGCCTCCTCAA |
| | CAC | | | | | GTGGGTCCCTGACCCCCCGTGCCTCCTG |
| | (SEQ ID | | | | | ACTGGGAGACAACTTCCAGCAGGGGGCG |
| | NO: 899) | | | | | ATAGACACCTTATACAGCAGAG |
| | | | | | | CTGTGGCTGGCATCTGGCAGGTGCCCCT |
| | | | | | | CTGGGATGAAGCTGCCGGAGCAAGGAG |
| | | | | | | CAGGCAGGAATTTTTGCTGTTCTGCAGCC |
| | | | | | | TCCGCTGGTGATAACCAGGCAAACAGGG |
| | | | | | | TCTGGAGTGGACCTTCAGCAAACTCCAG |
| | | | | | | C (SEQ ID NO: 982) |
| 30 | GTCTG | 61.508 | 368 | 235 | 133 | GCGCCTGGCTCAAGACGAAGATTTAACC |
| | GGGAA | | | | | GAGAACAAAAGAACGTTTGCCAATCAGAA |
| | CTGGG | | | | | AACGCTACCCGAGAACGAGGATAACCCC |
| | GTGAC | | | | | GCTTTGTGTTTTGAAAACTCTTTAATTAGC |
| | T (SEQ | | | | | CTGGTTTCTAAGACGCATTAAAACATTCCT |
| | ID NO: | | | | | ACGCAGATTCTAAATGGAGGATCAAATGG |
| | 900) | | | | | TTGCTTCTGCAACGAAGATGGGATCCCGA |
| | | | | | | AAGGGCACACGCCGCAGAACCAAGGCAC |
| | | | | | | ACGGGGCCACTGAGACGCCCGGCGCGT |
| | | | | | | CGTACAGAGTGTTCCTGGGCGCTCGGTC |
| | | | | | | TCAGGTTACTTCTAGAACCAGTCTCCCCA |
| | | | | | | ACGCCGTCTTCGTTTCCTTAAAACCTCGC |
| | | | | | | TAGAGTCACCCCAGTTCCCCAGAC (SEQ |
| | | | | | | ID NO: 983) |
| 31 | AGATG | 61.088 | 331 | 128 | 203 | TCTGTGGGCCCCTCAAGAGGACCTGACA |
| | TGGCT | | | | | TCTTCTCATTCATCTCTGCCTTGTTTAACA |
| | AAGAC | | | | | GCTGGGCAAATTGAGGCCCAGAGAGGG |
| | CACGT | | | | | GCAATGACTTGCCCAGGGTCACGCAGCG |
| | GGG | | | | | AGTCTGGACAGGTAGTCCAGGGCCCCCG |
| | (SEQ ID | | | | | GGGTGTAGGCTCAGCAGCCAGCCCTAGT |
| | NO: | | | | | CAGGGACAGAACACAACCTGGTGACCTC |
| | 901) | | | | | TGCTTTGCCTCAGGTTCTAGACACAGGGT |
| | | | | | | GTTAGGACGGTTCTCGGTCTGAGGTCAG |
| | | | | | | TTCTGGGGCCGGCACCGGGGTGAGCCA |
| | | | | | | CGATCTCCCACTAGGGGGGGCCAAAAGC |
| | | | | | | CCACGTGGTCTTAGCCACATCT (SEQ ID |
| | | | | | | NO: 984) |
| 32 | GGGAT | 61.508 | 362 | 128 | 234 | CGGTCTTGGTTTCAGACCCGCCACTGAG |
| | CCAGG | | | | | CCCACACACCAACAGAGAGAAATATGCTG |
| | GCCAC | | | | | AAATGCTGTTTGAAGCCTTCAACACCCCT |
| | AAAGC | | | | | GCAATGCACATCGCCTACCAGTCGCGCC |
| | A (SEQ | | | | | TGTCCATGTACTCCTATGGAAGGACCTCC |
| | ID NO: | | | | | GGCCTGGTGGTGGAGGTGGGCCATGGC |
| | 902) | | | | | GTGTCCTACGTGGTCCCCATCTACGAGG |
| | | | | | | GTTATCCTTTGCCCAGCATCACCGGAAGG |
| | | | | | | CTGGACTACGCGGGCTCTGACCTGACAG |
| | | | | | | CCTACCTGCTGGGCCTGCTGAACAGTGC |
| | | | | | | GGGGAACGAATTCACCCAGGACCAGATG |
| | | | | | | GGCATCGTGGAGGACATCAAGAAGAAAT |
| | | | | | | GCTGCTTTGTGGCCCTGGATCCC (SEQ ID |
| | | | | | | NO: 985) |
| 33 | GAGCC | 61.508 | 340 | 126 | 214 | CCGAGATCACGCCAGTGCACTTCAGCCT |
| | ACCAT | | | | | GGGCGACTGAGTGAGGCTCCATCTCAAA |
| | GCCAG | | | | | AAAGAAAAAACTTTGGTACAGATAATCTG |
| | GTCAT | | | | | GCTCCTCCCTGGGCATCACCCCCGGGAG |
| | C (SEQ | | | | | CCTACTCCACTCCATTAGCCTACAGCCCT |
| | ID NO: | | | | | GCCTCTGACTTCAAACCCTAAGCATGATG |
| | 903) | | | | | GCCATGAATACTAAAAAAATACTCAACATC |
| | | | | | | AGTATCAACTGAGTACCCTTTCTAGGTAT |
| | | | | | | CTATATTAGACACTTTTTCTGCTGAAACCC |
| | | | | | | TTTTATGAGTAAAAAAAGGAACTGAATGTT |
| | | | | | | TATACAAAAGTTTATGTAAATCTTTAGATG |
| | | | | | | ATGACCTGGCATGGTGGCTC (SEQ ID NO: |
| | | | | | | 986) |
| 34 | GGCCT | 61.508 | 490 | 202 | 288 | GGGGAAGGGGAAAGAGGCAGTTAATATT |
| | CCTGG | | | | | TAACAAGCACAGAGTTTTTGTTGGGGATG |
| | GCTCA | | | | | ATTATGCAACATTATGACTGTATTTAATGC |
| | CATGA | | | | | CAGTGAATTGCACACTTACAAATCATTAAA |
| | T (SEQ | | | | | ATGATAAATATTAGGTTATAGTATATTTTAC |
| | ID NO: | | | | | CAACATTAAAAAAACCAACATGTATTAAAT |
| | 904) | | | | | CCACAGCTGGGCAGTCCTCTTGACCTTG |
| | | | | | | CAGACTGGCAGGAGTGAACCATCATTTAT |
| | | | | | | TACTTTGGGCTTCTCAGTAAAAAAAAAAAA |
| | | | | | | AATATATATATATATACACACACACACACA |
| | | | | | | CACACATATATATATATATACACATATATAT |
| | | | | | | ATATATATACCATAAGCAAAGTATTAGGGA |
| | | | | | | ACCAAAGGAACTAGACAATTTTATAAGGG |
| | | | | | | GGCCCAC |
| | | | | | | TTAAAATCCAGGCTGTTGAGCCAGGTGCA |
| | | | | | | GTGGCTTAAACCTATAATCCAAGCACCTT |
| | | | | | | TGGAGGCCAAGGCAAGAGGATCATGTGA |
| | | | | | | GCCCAGGAGGCC (SEQ ID NO: 987) |
| 35 | GGGTT | 61.508 | 418 | 250 | 168 | GCTGGACTGTCGCACCTCTGATCTCAGAT |
| | GAGGG | | | | | CAGGATGGAAAGGAAGGAACTGAGAGCT |
| | GACAC | | | | | AAAAAGAGAAATGGAGATCACTACGGTAG |
| | AGCAC | | | | | AACCAACTAACCTGGAGTTCAGCCGGTCA |
| | T (SEQ | | | | | TTTGGGAGACTATCCTGAGGGATTATCTT |
| | ID NO: | | | | | TTGGGGTCATGAGCAATTAGGATCTCTGA |
| | 905) | | | | | GATCTGCTGACTCCTGTGGCATCATCTGG |
| | | | | | | ACAGTATGCCTTCTCATCAGGGTACCCAG |
| | | | | | | GAGGCCTGTCCCCTCCTTCCCCGGGAGG |
| | | | | | | GACTCTGGCATGTCCTTCAAAACGCAGCT |
| | | | | | | CACACATAATCCCTGCTGAGCTTTCCAGA |
| | | | | | | ATGTCTCCAGAAAGAACTGTTTCCTCTGC |
| | | | | | | ACTTTTCGGTTGTTCTGGTAGCTCACTCTT |
| | | | | | | CGCCATAATTACTTATCTGCTAGTGCTGT |
| | | | | | | GTCCCCTCAACCC (SEQ ID NO: 988) |
| 36 | GTTCA | 61.508 | 331 | 127 | 204 | GTCCCCTCCACCCCTTAGACTCCAGCCA |
| | GGTGG | | | | | GATTCAGGCATTCCCTGCATCCCACGCCA |
| | TGCCA | | | | | ATGCATCACTGTCCCCATCCTAAGTATCG |
| | GGTGC | | | | | TGGATAAGCTGCAGTGACAAGCCCCAGG |
| | T (SEQ | | | | | GCCTCCTGTCGAAAACGGTGTCAGCCTG |
| | ID NO: | | | | | GCCGGGCACAGGCTGGTCTCCAAGACTC |
| | 906) | | | | | TGTGCCCTGGACTCAGCTCATAGCAGCT |
| | | | | | | CCTTCCTGTTGTTCAGGTCTCGATTTCAA |
| | | | | | | GGACACCATCGCAAAGACTGTCCCCAAG |
| | | | | | | CACTTCACAAAAGCTACCCACCTCCACCT |
| | | | | | | CTCACGCTGTCTGCGTCTCCCCTCAGAG |
| | | | | | | CACCTGGCACCACCTGAAC (SEQ ID NO: |
| | | | | | | 989) |
| 37 | CTGTG | 61.088 | 495 | 313 | 182 | CCTTCCTCTGTGGTGTGACTTTCAGTTGC |
| | GTTTC | | | | | TTCTAGATTTTACTCTTCTAACTCAACAGT |
| | AGGCA | | | | | TAATGCTTTTGTGGGATAAAATTTCCCTTC |
| | TCTGC | | | | | ATATTTAGGATTATTCCTTTAGGTTAACTC |
| | TGG | | | | | TCAGGCCTACTAGTCCTCTGATTCACACT |
| | (SEQ ID | | | | | GTCTTTTTAATTTTTCCCAATCTATTACATA |
| | NO: 907) | | | | | TTTTCTAAATTTTGTACACCATTCATATGTC |
| | | | | | | ATGTGTATCATATCAAAGAGCCTAGAGAA |
| | | | | | | AATTTTCCTCTGTGTGTTCCTCACTGGGT |
| | | | | | | GGTTACAATGGAGAAATTACAGGGCAGG |
| | | | | | | ACTAGCATTAGGGTGAGGAGAGCAAGGC |
| | | | | | | ACTCAGGCGCAAAACTTAAGGGGGCACC |
| | | | | | | AATATCCTCAGTGATCAAGAGAAGTAATAT |
| | | | | | | TGTCACGTAACTGTATTAGCTTTTTAGGG |
| | | | | | | CTACTACTGTAATGAAGTACAGTATTCTCT |
| | | | | | | CTTATCCATGGGGAAATGCATTCCAAAAC |
| | | | | | | CCCCAGCAGATGCCTGAAACCACAG |
| | | | | | | (SEQ ID NO: 990) |
| 38 | GCTGG | 61.088 | 347 | 130 | 217 | GGAGCAAGTGGTCCCTGTGGAAAGCCGC |
| | CAGCA | | | | | TGCACTAGGTGAGCCCGGCCCTGGTGAG |
| | GTTCT | | | | | GGTGCCTTGTGAGTGGAGCCTGCTTGTG |
| | GATGA | | | | | CTGCTGTGCCCCGCTGCTTCCAGGCGGC |
| | TCC | | | | | GCGGCCCTCCGTCTGATTGCTGGAGGAC |
| | (SEQ ID | | | | | TCTTCGGTGTGGACACACCGTGGTTTGTT |
| | NO: 908) | | | | | TACCCATCCTGGTCAATGGACAATTGGAA |
| | | | | | | CTGTGGTGAACAAGCATTCCTGCACCTGT |
| | | | | | | ATGTGGATATTGTATGTCCATTTTTCTTGG |
| | | | | | | GAGTGGAATGGCTGAGTCATCTGGTAGG |
| | | | | | | TGTCTTTACCTTTTAAAGAACTAAGCGCTT |
| | | | | | | TTTCCAGAAGGATCATCAGAACTGCTGCC |
| | | | | | | AGC (SEQ ID NO: 991) |
| 39 | GCCCG | 59.378 | 518 | 219 | 299 | GGGAATGCTGGCACTGTCTAGGATAGAA |
| | TTTCA | | | | | TGTTCCTAATTATAGTAGCTTACCCATTGA |
| | GTGCT | | | | | AGGCTGAAATAAAACATGCAAGGATGTGT |
| | ACTGT | | | | | GTGTTTGTGTGTGTGTGTGTATGTGTGTA |
| | G (SEQ | | | | | AAGGCTTTAGAAAGCTGATGAGACCTGAG |
| | ID NO: | | | | | GTACTCAATCTCCAAGAGAAGAAAACTGA |
| | 909) | | | | | GCACCCTGAGGACAGTTAG |
| | | | | | | GGAACAGGGCTGTTGAATCATAGCAGAC |
| | | | | | | AGCAACAGGGCCTCTAGTGTGCAAACAG |
| | | | | | | AATTGGAGTTCAGATCCTCCAAAGTGGCT |
| | | | | | | GTGATCTGAAAGACAAAACTGACATCAAA |
| | | | | | | GAATCCTCACAGAAGCGAGCGGAAATATT |
| | | | | | | TTTATATACTTTTACAAATCCTTTGCAAAAT |
| | | | | | | TCAAAACTTTGCATACAGAAGTCAAGACA |
| | | | | | | AAGTAAGATGCTCAGAAGGCTGTAATCCC |
| | | | | | | TGGAAGGCTAGAGAATTAAGCAGTATCAG |
| | | | | | | AGATGTAAGATGCTAGCAAGAACAAAGTT |
| | | | | | | GATTTTAATCTGAACACAGTAGCACTGAA |
| | | | | | | ACGGGC (SEQ ID NO: 992) |
| 40 | ATGCG | 61.508 | 343 | 217 | 126 | GGGCTTCTGGCATCACTGGGAGTCCTGG |
| | ATGGG | | | | | CCAGGCCCCAGGCACACCACAGTGGGG |
| | AGGCC | | | | | GACAGGAGCCCCAGCCCAGCTGGGAGA |
| | CAGAG | | | | | GGCAGGCCTCCTTGATCCCTATCAGCCC |
| | T (SEQ | | | | | GGATGTTGGGGAGGGGGTATGGTGCTG |
| | ID NO: | | | | | GTCGGGTCCTTGGGATCCCTAGGTGGCT |
| | 910) | | | | | CGGGGCTCCAGAGTACAGGCTCTGATAC |
| | | | | | | GTGACAGGCCTGTAGGTGGCCGGGGCC |
| | | | | | | AGCAGGACCTCAGGTGGGGGCAGCAGC |
| | | | | | | TGATGATGGAGCCAAGCAGGGGTCTTGG |
| | | | | | | AGGTGGAAGCTGAAGCTTTGTTGGGGTC |
| | | | | | | TGAGGGAGGAAATGAGTGGACTCTGGGC |
| | | | | | | CTCCCATCGCAT (SEQ ID NO: 993) |
| 41 | CATCT | 61.508 | 343 | 128 | 215 | CGCTCTTCCCAAGCTCCCGTTGGCCCTC |
| | TCGAC | | | | | ACCGTCGAAGTCAATGCGGCCGTCGTTG |
| | AGGTG | | | | | TTCTTGTCGCCGTCTTTCATCAGAGATTC |
| | CGCTG | | | | | GATCTCCTCGTCCGTCACGTGCTCCCCG |
| | G (SEQ | | | | | GAGGCCCTGAAAATCTCAGCCAGCTCCT |
| | ID NO: | | | | | CCGGGTCGATGTAGCCGTCTGCATTCCTT |
| | 911) | | | | | CAGGTCCGAGGGACAGGGCAGGGCTCA |
| | | | | | | GGGCCGGGGCGAGCTGCCCTGGCCACC |
| | | | | | | ACACTCGACGCCCCGCTTCCGCACTCCC |
| | | | | | | AACACGGGGAAGCTTCCAGCGCTGCCGG |
| | | | | | | CCGGGTTCTGACTGCTAAGCCCCTCCCT |
| | | | | | | CGGCTCCCGGGCCCCCAGCGCACCTGT |
| | | | | | | CGAAGATG (SEQ ID NO: 994) |
| 42 | ATTTG | 62.768 | 365 | 126 | 239 | GGTGTTCTCACACCCCAGGTGTCC |
| | CTGTG | | | | | TCACAAGGGGCTTGGGCGCAGAA |
| | CGAGG | | | | | CAGGGCTTGGGGGCGGGTAGCG |
| | CCTTC | | | | | GGGCAAAGCGTCTTCGGGGGGGG |
| | GCCA | | | | | AGATCAGTGACCCGGCAGGGAGG |
| | (SEQ ID | | | | | CCGCACCTGGTCCACGAGGCCCT |
| | NO: 912) | | | | | CGGGGTTGAGCAGCGTGGCCCTC |
| | | | | | | GCGATGGTGTTCACCTGCAGCGT |
| | | | | | | GTATCGAGTGTGGGGGAGTAGGA |
| | | | | | | GCTGCGAGCGGAGCGGATCACGT |
| | | | | | | GAGCTGAAGGCCGGGGACAAGGC |
| | | | | | | CGCCCGGCCCCTGCCCCGCTGGG |
| | | | | | | CCCCCACCCGGGGTCGCGGACCT |
| | | | | | | TGTAGATGGGGTGGCAGAGCGGC |
| | | | | | | AGCTGGCGCAGCGTGGCCATGGC |
| | | | | | | GAAGGCCTCGCACAGCAAAT (SEQ |
| | | | | | | ID NO: 995) |
| 43 | No | No | No | No | No | |
| | Primers | Primers | Primers | Primers | Primers | |
| 44 | CCCTG | 61.508 | 398 | 236 | 162 | TGGCCTCCCAGATTGCTGGGATTA |
| | GCTCA | | | | | TAGGCGTGAGCCAGGAGTGGTCA |
| | GAGCC | | | | | TTTTTGAAAAGGTCCTAAGCTTACT |
| | CTCAA | | | | | GATTTTTTTTTCCTAAAATCATCTG |
| | T (SEQ | | | | | GTTTGTTAAAAATTATTTCTTTAGA |
| | ID NO: | | | | | AGCTTAAAAAAATAACACAAAATAA |
| | 913) | | | | | AGCACTGTCCTTGTTCTGCAGGTG |
| | | | | | | ATCGGTGTCTTTGGTGAGTCTGTT |
| | | | | | | AAAGGCTATGGGGAGGTGACAAG |
| | | | | | | GCGGGGTCGCCGGCAGCATGTCA |
| | | | | | | GGTACTGTGGCTTGAATTCAGAGG |
| | | | | | | AGGCTCTGGGGAGATTTTTAAATG |
| | | | | | | AAAGGAACTTAGACTGATCTTTCG |
| | | | | | | TTTTCTCGACAG |
| | | | | | | GTTCACTGAATATGTAAAAGATGGGCTGA |
| | | | | | | AACACACGTGTGTGAAATTCTACATTGAG |
| | | | | | | GGCTCTGAGCCAGGG (SEQ ID NO: 996) |
| 45 | GCTGG | 61.508 | 395 | 126 | 269 | GCTTGGAGCTGGGCCATCAGATCAGATG |
| | CACAT | | | | | AAGAGGTAAGAAGAGCCTTGCAGTGGAC |
| | GGGGA | | | | | GCAGGGTGATCACCAAGGGAGGGGGGG |
| | GACAT | | | | | GAAGGCTAGCCAAGTCCAGGCTGCAGAG |
| | C (SEQ | | | | | CCATGGGGCACTGGCAGCAGTGAGACAG |
| | ID NO: | | | | | CCGCTCTGTGTGACCAGGCTGGGGAGG |
| | 914) | | | | | CAGTGCTCTGGGTCACCTGCCTGCGCTG |
| | | | | | | GGCTGGACGGCGAGATGACACCTTCATC |
| | | | | | | CCTCTTTCAGCCACACTGTCCAGAGTGAG |
| | | | | | | TTCACACCCTCATTATATCTCACCTGGAAC |
| | | | | | | ATTCCAGCAGCCCTTCACTTGTCCTCCCC |
| | | | | | | CACACACCAACATGATCTGTCTGAATCAC |
| | | | | | | AAATCTGATCCCACTTCTCCTTGATGATAA |
| | | | | | | CATTCGATGTCTCCCCATGTGCCAGC |
| | | | | | | (SEQ ID NO: 997) |
| 46 | CCTCT | 61.508 | 426 | 172 | 254 | CTGTACACACGCCCAGATGGCTGTTTAGA |
| | TTCGG | | | | | AAAGAACCTCACCCTGATTTCCAACAAGG |
| | ATGGG | | | | | TTTCCCCTGGAGAGTCTACGAGTTTTAGA |
| | CCCAT | | | | | GGCAGAGGCAGTTGTTGGAACTGTGTTC |
| | G (SEQ | | | | | ACCAGGATGAAGGAAGAAAGGAAGGGAT |
| | ID NO: | | | | | GTTCTCAGCAAAGAGCCTGGGCCTGGCC |
| | 915) | | | | | TGGTCCTTGCCAGTCCTGCCTCATGACAG |
| | | | | | | CCGCCACTGCCCGAGAGAGCCTCCCAGC |
| | | | | | | CCAGCCGCCTGCAGGGCATCCTCCGCCC |
| | | | | | | TGGGTTGGGGGCTGTGAGCTCACATGCT |
| | | | | | | TTGGGGACTACAGGGTCATGAAACTGAG |
| | | | | | | CAAAGGGGCTGGTGTAAGAGGATTGAGC |
| | | | | | | AATGGACACACTGCCTCGGCGGTCAGGG |
| | | | | | | ACAAAAGGGAGTGTGAGGACCGTGATGA |
| | | | | | | ACTGGAGCACATGGGCCCATCCGAAAGA |
| | | | | | | GG (SEQ ID NO: 998) |
| 47 | CAGGT | 61.508 | 360 | 137 | 223 | GCTTCTCCTGAAGACCACGTTTCCTGCCC |
| | CCGTC | | | | | GGTCGGCCTTCCACCCTTTCACCAGGGC |
| | GCATC | | | | | GAAGTCTGCCCGGATGGCGCGCTCCAAA |
| | GTCTG | | | | | AGGAAGTGGTCGCCGTTGAACTCCCTCA |
| | T (SEQ | | | | | CCTCTCGGGGCTGGCTCATGAGCGCCAG |
| | ID NO: | | | | | GTGGCCGTCCGGGGTGTAGCGGATGGG |
| | 916) | | | | | GGCGCCCCCTTCCTGGACCAGGGTCCC |
| | | | | | | GTAGCCCGTGGGGGTGTAGAAGGGGGG |
| | | | | | | CACCCCGGCGCCCCCCGCGCGGATGCG |
| | | | | | | CTCGGCCAGGGTGCCCTGGGGCGTGAG |
| | | | | | | CTCCAGCTCCAGCTCTCCTGCCAGGTACT |
| | | | | | | GGCTCTCGCACAGGGTGTTCTCGCCCAC |
| | | | | | | GTAGGAACAGACGATGCGACGGACCTG |
| | | | | | | (SEQ ID NO: 999) |
| 48 | GCTTC | 60.908 | 342 | 209 | 133 | TGTTCCTACGTGGGCGAGAACACCCTGT |
| | TCCTG | | | | | GCGAGAGCCAGTACCTGGCAGGAGAGCT |
| | AAGAC | | | | | GGAGCTGGAGCTCACGCCCCAGGGCAC |
| | CACGT | | | | | CCTGGCCGAGCGCATCCGCGGGGGGGG |
| | TTCC | | | | | CGCCGGGGTGCCCGCCTTCTACACCCCC |
| | (SEQ ID | | | | | ACGGGCTACGGGACCCTGGTCCAGGAA |
| | NO: | | | | | GGGGGCGCCCCCATCCGCTACACCCCG |
| | 817) | | | | | GACGGCCACCTGGCGCTCATGAGCCAGC |
| | | | | | | CCCGAGAGGTGAGGGAGTTCAACGGGG |
| | | | | | | ACCACTTCCTTTTGGAGCGCGCCATCCG |
| | | | | | | GGCAGACTTCGCCCTGGTGAAAGGGTGG |
| | | | | | | AAGGCCGACCGGGCAGGAAACGTGGTCT |
| | | | | | | TCAGGAGAAGC (SEQ ID NO: 1000) |
| 49 | GGTAA | 61.288 | 431 | 284 | 147 | CTGTTTTCCACAGTGGCCGAACCATTTTC |
| | GACTC | | | | | TATTTCCACCAGCAATGTAGCGAATTCCA |
| | ACCCA | | | | | ATTTCTCCATTTCCTCACCAATACTTGGTA |
| | GCCTG | | | | | TTGTCCTCTAAAAAAAGATCCTAGCTGTTC |
| | GA | | | | | TAGCGGGTGTGAGGTGGTATCTCATGGT |
| | (SEQ ID | | | | | GGTTTTGATTTGCATTTCCCTAATGGCTAA |
| | NO: 917) | | | | | TGATGCTGAGCATCTCTTCCTGTGCTTGT |
| | | | | | | TGGCTGTCTGTATATCGACTTTGGAGAGC |
| | | | | | | GTATTGATTTTGCATTCCTGGTGCCTCCT |
| | | | | | | GCAGGGCCTGGTGCGGACCCAGTGCTC |
| | | | | | | CA |
| | | | | | | GACGGCTCTGCTGAGTAAAGAACAGGAG |
| | | | | | | AATAAACAAGTTGTGTTCCCCTGCCGTGG |
| | | | | | | TCCACGCTGGGCTCCTTGTGGACATACCT |
| | | | | | | GTCTCGTGAGGCAGTGAGACCCCTTTGA |
| | | | | | | CATTCCAGGCTGGGTGAGTCTTACC (SEQ |
| | | | | | | ID NO: 1001) |
| 50 | CCCAC | 60.908 | 554 | 233 | 321 | CTGCACACTTGCACACACACAGGCAGGT |
| | AAGCT | | | | | ATAGGCACACAGTTCCACCAAAGCGAAGT |
| | CTTGA | | | | | CCAAATCAGAGTTCTTACCGATTCTACCA |
| | CTTCT | | | | | CTCTGTAATACTCTCAGAAGTTCCCACAA |
| | GTCC | | | | | CTGCCCAACACCTCACCTACCCGTTTCCT |
| | (SEQ ID | | | | | GCTGCACAAAGTCTCCTTCACTCTCATCT |
| | NO: | | | | | TTTCGATACATCGTCAGGCAGGGCAGAA |
| | 918) | | | | | GGGCACTACGCCGCAGAGCATTCGAGGC |
| | | | | | | TCTCAGCACACTGCCAGCCAGCGTGAAA |
| | | | | | | ATGCGGCTTCACTACAGCCCAGAGAAAA |
| | | | | | | GGGCCAGGTTTAGTCATCGATCCAGATG |
| | | | | | | CATTTTCCCGGGAAATGACCATTCCCAAA |
| | | | | | | CCCACAGAACAGTTTGGCTTCTCTGGATA |
| | | | | | | TCCTTATAGGAAATAGGGGTGAGAGGTG |
| | | | | | | GGAAAAGCTTCTCAATCTCATACAGAAAC |
| | | | | | | AGCACAAATATTTAATACACCTTTTGCAAC |
| | | | | | | AAGATCTTCTCTGTTCTCCATTAAGCATCA |
| | | | | | | TGTAAATCTCACTAGTGTGGTTAACAAAAT |
| | | | | | | GGCTTTCCCCAAGGACAGAAGTCAAGAG |
| | | | | | | CTTGTGGG (SEQ ID NO: 1002) |

### REFERENCES

The following references are each incorporated herein by reference in their entireties.
1. Jinek, M. et al. A Programmable Dual-RNA-Guided DNA Endonuclease in Adaptive Bacterial Immunity. Science 337, 816-821 (2012).
2. Cong, L. et al. Multiplex Genome Engineering Using CRISPR/Cas Systems. Science 339, 819-823 (2013).
3. Komor, A. C., Badran, A. H. & Liu, D. R. CRISPR-Based Technologies for the Manipulation of Eukaryotic Genomes. Cell 168, 20-36 (2017).
4. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
5. Nishida, K. et al. Targeted nucleotide editing using hybrid prokaryotic and vertebrate adaptive immune systems. Science 353, aaf8729 (2016).
6. Gaudelli, N. M. et al. Programmable base editing of A⋅T to G⋅C in genomic DNA without DNA cleavage. Nature 551, 464-471 (2017).
7. ClinVar, July 2019.
8. Dunbar, C. E. et al. Gene therapy comes of age. Science 359, eaan4672 (2018).
9. Cox, D. B. T., Platt, R. J. & Zhang, F. Therapeutic genome editing: prospects and challenges. Nat. Med. 21, 121-131 (2015).
10. Adli, M. The CRISPR tool kit for genome editing and beyond. Nat. Commun. 9, 1911 (2018).
11. Kleinstiver, B. P. et al. Engineered CRISPR-Cas9 nucleases with altered PAM specificities. Nature 523, 481-485 (2015).
12. Kleinstiver, B. P. et al. High-fidelity CRISPR-Cas9 nucleases with no detectable genome-wide off-target effects. Nature 529, 490-495 (2016).
13. Hu, J. H. et al. Evolved Cas9 variants with broad PAM compatibility and high DNA specificity. Nature 556, 57-63 (2018).
14. Nishimasu, H. et al. Engineered CRISPR-Cas9 nuclease with expanded targeting space.

### Science 361, 1259-1262 (2018).

15. Jasin, M. & Rothstein, R. Repair of strand breaks by homologous recombination. Cold Spring Harb. Perspect. Biol. 5, a012740 (2013).
16. Paquet, D. et al. Efficient introduction of specific homozygous and heterozygous mutations using CRISPR/Cas9. Nature 533, 125-129 (2016).
17. Kosicki, M., Tomberg, K. & Bradley, A. Repair of double-strand breaks induced by CRISPR-Cas9 leads to large deletions and complex rearrangements. Nat. Biotechnol. 36, 765-771 (2018).
18. Haapaniemi, E., Botla, S., Persson, J., Schmierer, B. & Taipale, J. CRISPR-Cas9 genome editing induces a p53-mediated DNA damage response. Nat. Med. 24, 927-930 (2018).
19. Ihry, R. J. et al. p53 inhibits CRISPR-Cas9 engineering in human pluripotent stem cells. Nat. Med. 24, 939-946 (2018).
20. Richardson, C. D., Ray, G. J., DeWitt, M. A., Curie, G. L. & Corn, J. E. Enhancing homology-directed genome editing by catalytically active and inactive CRISPR-Cas9 using asymmetric donor DNA. Nat. Biotechnol. 34, 339-344 (2016).
21. Srivastava, M. et al. An Inhibitor of Nonhomologous End-Joining Abrogates Double-Strand Break Repair and Impedes Cancer Progression. Cell 151, 1474-1487 (2012).
22. Chu, V. T. et al. Increasing the efficiency of homology-directed repair for CRISPR-Cas9-induced precise gene editing in mammalian cells. Nat. Biotechnol. 33, 543-548 (2015).
23. Maruyama, T. et al. Increasing the efficiency of precise genome editing with CRISPR-Cas9 by inhibition of nonhomologous end joining. Nat. Biotechnol. 33, 538-542 (2015).
24. Kim, Y. B. et al. Increasing the genome-targeting scope and precision of base editing with engineered Cas9-cytidine deaminase fusions. Nat. Biotechnol. 35, 371-376 (2017).
25. Li, X. et al. Base editing with a Cpf1-cytidine deaminase fusion. Nat. Biotechnol. 36, 324-327 (2018).
26. Gehrke, J. M. et al. An APOBEC3A-Cas9 base editor with minimized bystander and off-target activities. Nat. Biotechnol. (2018). doi:10.1038/nbt.4199
27. Rees, H. A. & Liu, D. R. Base editing: precision chemistry on the genome and transcriptome of living cells. Nat. Rev. Genet. 1 (2018). doi:10.1038/s41576-018-0059-1.
28. Ostertag, E. M. & Kazazian Jr, H. H. Biology of Mammalian L1 Retrotransposons. Annu. Rev. Genet. 35, 501-538 (2001).
29. Zimmerly, S., Guo, H., Perlman, P. S. & Lambowltz, A. M. Group II intron mobility occurs by target DNA-primed reverse transcription. Cell 82, 545-554 (1995).
30. Luan, D. D., Korman, M. H., Jakubczak, J. L. & Eickbush, T. H. Reverse transcription of R2Bm RNA is primed by a nick at the chromosomal target site: a mechanism for non-LTR retrotransposition. Cell 72, 595-605 (1993).
31. Feng, Q., Moran, J. V., Kazazian, H. H. & Boeke, J. D. Human L1 retrotransposon encodes a conserved endonuclease required for retrotransposition. Cell 87, 905-916 (1996).
32. Jinek, M. et al. Structures of Cas9 Endonucleases Reveal RNA-Mediated Conformational Activation. Science 343, 1247997 (2014).
33. Jiang, F. et al. Structures of a CRISPR-Cas9 R-loop complex primed for DNA cleavage. Science aad8282 (2016). doi:10.1126/science.aad8282
34. Qi, L. S. et al. Repurposing CRISPR as an RNA-Guided Platform for Sequence-Specific Control of Gene Expression. Cell 152, 1173-1183 (2013).
35. Tang, W., Hu, J. H. & Liu, D. R. Aptazyme-embedded guide RNAs enable ligand-responsive genome editing and transcriptional activation. Nat. Commun. 8, 15939 (2017).
36. Shechner, D. M., Hacisuleyman, E., Younger, S. T. & Rinn, J. L. Multiplexable, locus-specific targeting of long RNAs with CRISPR-Display. Nat. Methods 12, 664-670 (2015).
37. Anders, C. & Jinek, M. Chapter One—In vitro Enzymology of Cas9. in Methods in Enzymology (eds. Doudna, J. A. & Sontheimer, E. J.) 546, 1-20 (Academic Press, 2014).
38. Briner, A. E. et al. Guide RNA Functional Modules Direct Cas9 Activity and Orthogonality. Mol. Cell 56, 333-339 (2014).
39. Nowak, C. M., Lawson, S., Zerez, M. & Bleris, L. Guide RNA engineering for versatile Cas9 functionality. Nucleic Acids Res. 44, 9555-9564 (2016).
40. Sternberg, S. H., Redding, S., Jinek, M., Greene, E. C. & Doudna, J. A. DNA interrogation by the CRISPR RNA-guided endonuclease Cas9. Nature 507, 62-67 (2014).
41. Mohr, S. et al. Thermostable group II intron reverse transcriptase fusion proteins and their use in cDNA synthesis and next-generation RNA sequencing. RNA 19, 958-970 (2013).
42. Stamos, J. L., Lentzsch, A. M. & Lambowitz, A. M. Structure of a Thermostable Group II Intron Reverse Transcriptase with Template-Primer and Its Functional and Evolutionary Implications. Mol. Cell 68, 926-939.e4 (2017).
43. Zhao, C. & Pyle, A. M. Crystal structures of a group II intron maturase reveal a missing link in spliceosome evolution. Nat. Struct. Mol. Biol. 23, 558-565 (2016).
44. Zhao, C., Liu, F. & Pyle, A. M. An ultraprocessive, accurate reverse transcriptase encoded by a metazoan group II intron. RNA 24, 183-195 (2018).
45. Ran, F. A. et al. Genome engineering using the CRISPR-Cas9 system. Nat. Protoc. 8, 2281-2308 (2013).
46. Liu, Y., Kao, H.-I. & Bambara, R. A. Flap endonuclease 1: a central component of DNA metabolism. Annu. Rev. Biochem. 73, 589-615 (2004).
47. Krokan, H. E. & Bjsris, M. Base Excision Repair. Cold Spring Harb. Perspect. Biol. 5, (2013).
48. Kelman, Z. PCNA: structure, functions and interactions. Oncogene 14, 629-640 (1997).
49. Choe, K. N. & Moldovan, G.-L. Forging Ahead through Darkness: PCNA, Still the Principal Conductor at the Replication Fork. Mol. Cell 65, 380-392 (2017).
50. Li, X., Li, J., Harrington, J., Lieber, M. R. & Burgers, P. M. Lagging strand DNA synthesis at the eukaryotic replication fork involves binding and stimulation of FEN-1 by proliferating cell nuclear antigen. J. Biol. Chem. 270, 22109-22112 (1995).
51. Tom, S., Henricksen, L. A. & Bambara, R. A. Mechanism whereby proliferating cell nuclear antigen stimulates flap endonuclease 1. J. Biol. Chem. 275, 10498-10505 (2000).
52. Tanenbaum, M. E., Gilbert, L. A., Qi, L. S., Weissman, J. S. & Vale, R. D. A protein-tagging system for signal amplification in gene expression and fluorescence imaging. Cell 159, 635-646 (2014).
53. Bertrand, E. et al. Localization of ASHI mRNA particles in living yeast. Mol. Cell 2, 437-445 (1998).
54. Dahlman, J. E. et al. Orthogonal gene knockout and activation with a catalytically active Cas9 nuclease. Nat. Biotechnol. 33, 1159-1161 (2015).
55. Tsai, S. Q. et al. GUIDE-seq enables genome-wide profiling of off-target cleavage by CRISPR-Cas nucleases. Nat. Biotechnol. 33, 187-197 (2015).
56. Tsai, S. Q. et al. CIRCLE-seq: a highly sensitive in vitro screen for genome-wide CRISPR-Cas9 nuclease off-targets. Nat. Methods 14, 607-614 (2017).
57. Schek N, Cooke C, Alwine J C. Molecular and Cellular Biology. (1992).
58. Gil A, Proudfoot N J. Cell. (1987).
59. Zhao, B. S., Roundtree, I. A., He, C. Nat Rev Mol Cell Biol. (2017).
60. Rubio, M. A. T., Hopper, A. K. Wiley Interdiscip Rev RNA (2011).
61. Shechner, D. M., Hacisuleyman E., Younger, S. T., Rinn, J. L. Nat Methods. (2015).
62. Paige, J. S., Wu, K. Y., Jaffrey, S. R. Science (2011).
63. Ray D., . . . Hughes T R. Nature (2013).
64. Chadalavada, D. M., Cerrone-Szakal, A. L., Bevilacqua, P. C. RNA (2007).
65. Forster A C, Symons R H. Cell. (1987).
66. Weinberg Z, Kim P B, Chen T H, Li S, Harris K A, Lunse C E, Breaker R R. Nat. Chem. Biol. (2015).
67. Feldstein P A, Buzayan J M, Bruening G. Gene (1989).
68. Saville B J, Collins R A. Cell. (1990).
69. Winkler W C, Nahvi A, Roth A, Collins J A, Breaker R R. Nature (2004).
70. Roth A, Weinberg Z, Chen A G, Kim P G, Ames T D, Breaker R R. Nat Chem Biol. (2013).
71. Choudhury R, Tsai Y S, Dominguez D, Wang Y, Wang Z. Nat Commun. (2012).
72. MacRae I J, Doudna J A. Curr Opin Struct Biol. (2007).
73. Bernstein E, Caudy A A, Hammond S M, Hannon G J Nature (2001).
74. Filippov V, Solovyev V, Filippova M, Gill S S. Gene (2000).
75. Cadwell R C and Joyce G F. PCR Methods Appl. (1992).
76. McInerney P, Adams P, and Hadi M Z. Mol Biol Int. (2014).
77. Esvelt K M, Carlson J C, and Liu D R. Nature. (2011).
78. Naorem S S, Hin J, Wang S, Lee W R, Heng X, Miller J F, Guo H. Proc Natl Acad Sci USA (2017).
79. Martinez M A, Vartanian J P, Wain-Hobson S. Proc Natl Acad Sci USA (1994).
80. Meyer A J, Ellefson J W, Ellington A D. Curr Protoc Mol Biol. (2014).
81. Wang H H, Isaacs F J, Carr P A, Sun Z Z, Xu G, Forest C R, Church G M. Nature. (2009).
82. Nyerges Á et al. Proc Natl Acad Sci USA. (2016).
83. Mascola J R, Haynes B F. Immunol Rev. (2013).
84. X. Wen, K. Wen, D. Cao, G. Li, R. W. Jones, J. Li, S. Szu, Y. Hoshino, L. Yuan, Inclusion of a universal tetanus toxoid CD4(+) T cell epitope P2 significantly enhanced the immunogenicity of recombinant rotavirus ΔVP8* subunit parenteral vaccines. Vaccine 32, 4420-4427 (2014).
85. G. Ada, D. Isaacs, Carbohydrate-protein conjugate vaccines. Clin Microbiol Infect 9, 79-85 (2003).
86. E. Malito, B. Bursulaya, C. Chen, P. L. Surdo, M. Picchianti, E. Balducci, M. Biancucci, A. Brock, F. Berti, M. J. Bottomley, M. Nissum, P. Costantino, R. Rappuoli, G. Spraggon, Structural basis for lack of toxicity of the diphtheria toxin mutant CRM197. Proceedings of the National Academy of Sciences 109, 5229 (2012).
87. J. de Wit, M. E. Emmelot, M. C. M. Poelen, J. Lanfermeijer, W. G. H. Han, C. van Els, P. Kaaijk, The Human CD4(+) T Cell Response against Mumps Virus Targets a Broadly Recognized Nucleoprotein Epitope. J Virol 93, (2019).
88. M. May, C. A. Rieder, R. J. Rowe, Emergent lineages of mumps virus suggest the need for a polyvalent vaccine. Int J Infect Dis 66, 1-4 (2018).
89. M. Ramamurthy, P. Rajendiran, N. Saravanan, S. Sankar, S. Gopalan, B. Nandagopal, Identification of immunogenic B-cell epitope peptides of rubella virus E1 glycoprotein towards development of highly specific immunoassays and/or vaccine. Conference Abstract, (2019).
90. U. S. F. Tambunan, F. R. P. Sipahutar, A. A. Parikesit, D. Kerami, Vaccine Design for H5N1 Based on B- and T-cell Epitope Predictions. Bioinform Biol Insights 10, 27-35 (2016).
91. Asante, E A. et. al. “A naturally occurring variant of the human prion protein completely prevents prion disease”. Nature. (2015).
92. Crabtree, G. R. & Schreiber, S. L. Three-part inventions: intracellular signaling and induced proximity. Trends Biochem. Sci. 21, 418-22 (1996).
93. Liu, J. et al. Calcineurin Is a Common Target of A and FKBP-FK506 Complexes. Cell 66, 807-815 (1991).
94. Keith, C. T. et al. A mammalian protein targeted by GI-arresting rapamycin-receptor complex. Nature 369, 756-758 (2003).
95. Spencer, D. M., Wandless, T. J., Schreiber, S. L. S. & Crabtree, G. R. Controlling signal transduction with synthetic ligands. Science 262, 1019-24 (1993).
96. Pruschy, M. N. et al. Mechanistic studies of a signaling pathway activated by the organic dimerizer FK1012. Chem. Biol. 1, 163-172 (1994).
97. Spencer, D. M. et al. Functional analysis of Fas signaling in vivo using synthetic inducers of dimerization. Curr. Biol. 6, 839-847 (1996).
98. Belshaw, P. J., Spencer, D. M., Crabtree, G. R. & Schreiber, S. L. Controlling programmed cell death with a cyclophilin-cyclosporin-based chemical inducer of dimerization. Chem. Biol. 3, 731-738 (1996).
99. Yang, J. X., Symes, K., Mercola, M. & Schreiber, S. L. Small-molecule control of insulin and PDGF receptor signaling and the role of membrane attachment. Curr. Biol. 8, 11-18 (1998).
100. Belshaw, P. J., Ho, S. N., Crabtree, G. R. & Schreiber, S. L. Controlling protein association and subcellular localization with a synthetic ligand that induces heterodimerization of proteins. Proc. Natl. Acad. Sci. 93, 4604-4607 (2002).
101. Stockwell, B. R. & Schreiber, S. L. Probing the role of homomeric and heteromeric receptor interactions in TGF-β signaling using small molecule dimerizers. Curr. Biol. 8, 761-773 (2004).
102. Spencer, D. M., Graef, I., Austin, D. J., Schreiber, S. L. & Crabtree, G. R. A general strategy for producing conditional alleles of Src-like tyrosine kinases. Proc. Natl. Acad. Sci. 92, 9805-9809 (2006).
103. Holsinger, L. J., Spencer, D. M., Austin, D. J., Schreiber, S. L. & Crabtree, G. R. Signal transduction in T lymphocytes using a conditional allele of Sos. Proc. Natl. Acad. Sci. 92, 9810-9814 (2006).
104. Myers, M. G. Insulin Signal Transduction and the IRS Proteins. Annu. Rev. Pharmacol. Toxicol. 36, 615-658 (1996).
105. Watowich, S. S. The erythropoietin receptor: Molecular structure and hematopoietic signaling pathways. J. Investig. Med. 59, 1067-1072 (2011).
106. Blau, C. A., Peterson, K. R., Drachman, J. G. & Spencer, D. M. A proliferation switch for genetically modified cells. Proc. Natl. Acad. Sci. 94, 3076-3081 (2002).
107. Clackson, T. et al. Redesigning an FKBP-ligand interface to generate chemical dimerizers with novel specificity. Proc. Natl. Acad. Sci. 95, 10437-10442 (1998).
108. Diver, S. T. & Schreiber, S. L. Single-step synthesis of cell-permeable protein dimerizers that activate signal transduction and gene expression. J. Am. Chem. Soc. 119, 5106-5109 (1997).
109. Guo, Z. F., Zhang, R. & Liang, F. Sen. Facile functionalization of FK506 for biological studies by the thiol-ene ‘click’ reaction. RSC Adv. 4, 11400-11403 (2014).
110. Robinson, D. R., Wu, Y.-M. & Lin, S.-F. The protein tyrosine kinase family of the human genome. Oncogene 19, 5548-5557 (2000).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A non-naturally occurring polypeptide DddA variant comprising an amino acid sequence of any one of SEQ ID NOs: 28-54, or an amino acid sequence having at least 85%, 90%, 95%, 96%, 97%, 98%, 99%, or 100% sequence identity with any of SEQ ID NOs: 28-54.

2. The non-naturally occurring polypeptide DddA variant of claim 1 which corresponds to an N-terminal half of the canonical DddA protein.

3. The non-naturally occurring polypeptide DddA variant of claim 1 which corresponds to an C-terminal half of the canonical DddA protein.

4. The non-naturally occurring polypeptide DddA variant of claim 1, wherein the variant is derived from a starter DddA protein using a continuous evolution process.

5. The non-naturally occurring polypeptide DddA variant of claim 4, wherein the continuous evolution process is PACE.

6. The non-naturally occurring polypeptide DddA variant of claim 4, wherein the starter DddA protein comprises SEQ ID NO: 25 and corresponds to the DddAtox peptide.

7. A nucleotide sequence comprising any one of the non-naturally occurring polypeptide DddA variants of claims 1-6.

8. A vector comprising the nucleotide sequence of claim 7.

9. A cell comprising the vector of claim 8.

10. A base editor comprising any one of the non-naturally occurring polypeptide DddA variants of claims 1-6.

11. A base editor comprising a heterodimer having first and second monomers, said first monomer comprising a first programmable DNA binding protein and the polypeptide DddA variant of claim 2, and said second monomer comprising a second programmable DNA binding protein and the polypeptide DddA variant of claim 3, wherein dimerization of the first and second monomers reconstitutes a double-stranded DNA deaminase activity of a complex comprising the polypeptide DddA variants of claims 2 and 3.

12. The base editor of claim 11, wherein the first and/or second programmable DNA binding protein are the same.

13. The base editor of claim 11, wherein the first and/or second programmable DNA binding protein are different.

14. The base editor of claim 11, wherein the first and/or second programmable DNA binding protein is a nucleic acid programmable DNA binding protein (napDNAbp).

15. The base editor of claim 14, wherein the napDNAbp is a Cas9 domain.

16. The base editor of claim 14, wherein the napDNAbp is a nickase.

17. The base editor of claim 14, wherein the napDNAbp comprises an inactivated nuclease activity.

18. The base editor of claim 14, wherein the napDNAbp is selected from the group consisting of: Cas9, Cas12e, Cas12d, Cas12a, Cas12b1, Cas3a, Cas12c, and Argonaute and optionally has a nickase activity.

19. The base editor of claim 14, wherein the napDNAbp comprises an amino acid sequence selected from the group consisting of SEQ ID NOs: 59-112, or an amino acid sequence having at least 90% sequence identity to an amino acid sequence selected from the group consisting of: SEQ ID NO: 59-112.

20. The base editor of claim 11, wherein the programmable DNA binding protein is a TALE protein.

21. The base editor of claim 20, wherein TALE protein comprises an amino acid sequence selected from the group consisting of SEQ ID NO: 1-12, or an amino acid sequence having at least 90% sequence identity with an amino acid sequence selected from the group consisting of SEQ ID NO: 1-12.

22. The base editor of claim 11, wherein the programmable DNA binding protein is a zinc finger protein.

23. The base editor of claim 22, wherein zinc finger protein is a commercially available zinc finger protein.

24. The base editor of claim 11, wherein the programmable DNA binding protein is a mitoTALE protein.

25. The base editor of claim 24, wherein mitoTALE protein comprises an amino acid sequence selected from the group consisting of: SEQ ID NO: 1-12, or an amino acid sequence having at least 90% sequence identity with an amino acid sequence selected from the group consisting of SEQ ID NO: 1-12.

26. The base editor of any one of claims 11-25, further comprising a linker that joins the non-naturally occurring polypeptide DddA variant with the programmable DNA binding protein.

27. The base editor of claim 26, wherein the linker comprises an amino acid sequence selected from the group consisting of: SEQ ID NOs: 202-222, or an amino acid sequence having at least 90% sequence identity to SEQ ID NO: 202-222.

28. The base editor of claim 26, wherein the linker comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids.

29. The base editor of any one of claims 11-25, further comprising one or more uracil glycosylase inhibitor (UGI) domains.

30. The base editor of claim 29, wherein the one or more UGI domains comprise an amino acid sequence selected from the group consisting of: SEQ ID NOs: 377-383, or an amino acid sequence having at least 90% sequence identity to SEQ ID NOs: 377-383.

31. A method of editing a target nucleotide sequence at a target site, comprising contacting a target nucleotide sequence with a base editor of any one of claims 11-30, thereby inducing deamination of a target base at the target site.

32. The method of claim 31, wherein the target base is a C.

33. The method of claim 32, wherein the C is within a 5′-TC-3′ sequence context.

34. The method of claim 33, wherein the C is within a 5′-TCC-3′ sequence context.

35. The method of claim 31, wherein the programmable DNA binding protein of the base editor is a TALE, mitoTALE, zinc finger protein, or napDNAbp.

36. The method of claim 31, wherein the editing occurs in vivo.

37. The method of claim 31, wherein the editing occurs ex vivo.

38. The method of claim 31, wherein the target nucleotide sequence is a disease gene.

39. The method of claim 31, wherein the target nucleotide sequence is a disease gene in a mitochondria.

40. The method of claim 31, wherein the method of editing a nucleotide sequence results in the treatment of a mitochondrial disease.
